SEQUENCE LISTING (1075 sequences)

SEQ ID NO: 1 - DNA, 3570 bp, Alphatorquevirus sp.
  source 1..3570: mol_type = genomic DNA
attttgtgcagcccgccaattctcgttcaaacaggccaatcaggaggctctacgtacacttcctggggtgtgtcttcgaagagtatataagcagaggcggtgacgaatggtagagtttttcctggcccgtccgcggcgagagcgcgagcggagcgagcgatcgagcgtcccgtgggcgggtgccgtaggtgagtttacacaccgcagtcaaggggcaattcgggctcgggactggccgggctatgggcaagattcttaaaaaattcccccgatccctctgtcgccaggacataaaaacatgccgtggagaccgccggtgcatagtgtccaggggcgagaggatcagtggttcgcgagcttttttcacggccacgcttcattttgcggttgcggtgacgctgttggccatcttaatagcattgctcctcgctttcctcgcgccggtccaccaaggccccctccggggctagagcagcctaaccccccgcagcagggcccggccgggcccggagggccgcccgccatcttggcgctgccggctccgcccgcggagcctgacgacccgcagccacggcgtggtggtggggacggtggcgccgccgctggcgccgcaggcgaccgtggagaccgagactacgacgaagaagagctagacgagcttttccgcgccgccgccgaagacgatttgtaagtaggagatggcgccggccttacaggcgcaggaggagacgcgggcgacgcagacgcagacgcagacgcagacataagcccaccctagtactcagacagtggcaacctgacgttatcagacactgtaagataacaggacggatgcccctcattatctgtggaaaggggtccacccagttcaactacatcacccacgcggacgacatcacccccaggggagcctcctacgggggcaacttcacaaacatgactttctccctggaggcaatatacgaacagtttctgtaccacagaaacaggtggtcagcctccaaccacgacctcgaactctgcagatacaagggtaccaccctaaaactgtacaggcacccagatgtagactacatagtcacctacagcagaacgggaccctttgagatcagccacatgacctacctcagcactcacccccttctcatgctgctaaacaaacaccacatagtggtgcccagcctaaagactaagcccaggggcagaaaggccataaaagtcagaataagaccccccaaactcatgaacaacaagtggtacttcaccagagacttctgtaacataggcctcttccagctctgggccacaggcttagaactcagaaacccctggctcagaatgagcaccctgagcccctgcataggcttcaatgtccttaaaaacagcatttacacaaacctcagcaacctacctcagcacagagaagacagacttaacattattaacaacacattacacccacatgacataacaggaccaaacaataaaaaatggcagtacacatataccaaactcatggcccccatttactattcagcaaacagggccagcacctatgacttactacgagagtatggcctctacagtccatactacctaaaccccacaaggataaaccttgactggatgaccccctacacacacgtcaggtacaatccactagtagacaagggcttcggaaacagaatatacatacagtggtgctcagaggcagatgtaagctacaacaggactaaatccaagtgtctcttacaagacatgcccctgtttttcatgtgctatggctacatagactgggcaattaaaaacacaggggtctcctcactagcgagagacgccagaatctgcatcaggtgtccctacacagagccacagctggtgggctccacagaagacatagggttcgtacccatcacagagaccttcatgaggggcgacatgccggtacttgcaccatacataccgttgagctggttttgcaagtggtatcccaacatagctcaccagaaggaagtacttgaggcaatcatttcctgcagccccttcatgccccgtgaccagggcatgaacggttgggatattacaataggttacaaaatggacttcttatggggcggttcccctctcccctcacagccaatcgacgacccctgccagcagggaacccacccgattcccgaccccgataagcaccctcgcctcctacaagtgtcgaacccgaaactgctcggaccgaggacagtgttccacaagtgggacatcagacgtgggcagtttagcaaaagaagtattaaaagagtgtcagaatactcatcggatgatgaatctcttgcgccaggtctcccatcaaagcgaaacaagctcgactcggccttcagaggagaaaacccagagcaaaaagaatgctattctctcctcaaagcactcgaggaagaagagaccccagaagaagaagaaccagcaccccaagaaaaagcccagaaagaggagctactccaccagctccagctccagagacgccaccagcgagtcctcagacgagggctcaagctcgtctttacagacatcctccgactccgccagggagtccactggaaccccgagctcacatagagcccccaccttacataccagacctactttttcccaatactggtaaaaaaaaaaaattctctcccttcgactgggaaacggaggcccagctagcagggatattcaagcgtcctatgcgcttctatccctcagacacccctcactacccgtggttaccccccaagcgcgatatcccgaaaatatgtaacataaacttcaaaataaagctgcaagagtgagtgattcgaggccctcctctgttcacttagcggtgtctacctcttaaagtcaccaagcactccgagcgtcagcgaggagtgcgaccctccaccaaggggcaacttcctcggggtccggcgctacgcgcttcgcgctgcgccggacgcctcggacccccccccgacccgaatcgctcgcgcgattcggacctgcggcctcggggggggtcgggggctttactaaacagactccgagttgccactggactcaggagctgtgaatcagtaacgaaagtgagtggggccagacttcgccatagggcctttaacttggggtcgtctgtcggtggcttccgggtccgcctgggcgccgccattttagctttagacgccattttaggccctcgcgggcacccgtaggcgcgttttaatgacgtcacggcagccattttgtcgtgacgtttgagacacgtgatgggggcgtgcctaaacccggaagcatccctggtcacgtgactctgacgtcacggcggccattttgtgctgtccgccatcttgtgacttccttccgctttttcaaaaaaaaagaggaagtatgacagtagcggcgggggggcggccgcgttcgcgcgccgcccaccagggggtgctgcgcgcccccccccgcgcatgcgcggggcccccccccgggggggctccgcccccccggcccccccccgtgctaaacccaccgcgcatgcgcgaccacgcccccgccgcc

SEQ ID NO: 2 - protein, 130 aa, Alphatorquevirus sp.
MPWRPPVHSVQGREDQWFASFFHGHASFCGCGDAVGHLNSIAPRFPRAGPPRPPPGLEQPNPPQQGPAGPGGPPAILALPAPPAEPDDPQPRRGGGDGGAAAGAAGDRGDRDYDEEELDELFRAAAEDDL

SEQ ID NO: 3 - protein, 303 aa, Alphatorquevirus sp.
MPWRPPVHSVQGREDQWFASFFHGHASFCGCGDAVGHLNSIAPRFPRAGPPRPPPGLEQPNPPQQGPAGPGGPPAILALPAPPAEPDDPQPRRGGGDGGAAAGAAGDRGDRDYDEEELDELFRAAAEDDFQSTTPASREPTRFPTPISTLASYKCRTRNCSDRGQCSTSGTSDVGSLAKEVLKECQNTHRMMNLLRQVSHQSETSSTRPSEEKTQSKKNAILSSKHSRKKRPQKKKNQHPKKKPRKRSYSTSSSSRDATSESSDEGSSSSLQTSSDSARESTGTPSSHRAPTLHTRPTFSQYW

SEQ ID NO: 4 - protein, 293 aa, Alphatorquevirus sp.
MPWRPPVHSVQGREDQWFASFFHGHASFCGCGDAVGHLNSIAPRFPRAGPPRPPPGLEQPNPPQQGPAGPGGPPAILALPAPPAEPDDPQPRRGGGDGGAAAGAAGDRGDRDYDEEELDELFRAAAEDDLSPIKAKQARLGLQRRKPRAKRMLFSPQSTRGRRDPRRRRTSTPRKSPERGATPPAPAPETPPASPQTRAQARLYRHPPTPPGSPLEPRAHIEPPPYIPDLLFPNTGKKKKFSPFDWETEAQLAGIFKRPMRFYPSDTPHYPWLPPKRDIPKICNINFKIKLQE

SEQ ID NO: 5 - protein, 180 aa, Alphatorquevirus sp.
MPWRPPVHSVQGREDQWSPIKAKQARLGLQRRKPRAKRMLFSPQSTRGRRDPRRRRTSTPRKSPERGATPPAPAPETPPASPQTRAQARLYRHPPTPPGSPLEPRAHIEPPPYIPDLLFPNTGKKKKFSPFDWETEAQLAGIFKRPMRFYPSDTPHYPWLPPKRDIPKICNINFKIKLQE

SEQ ID NO: 6 - protein, 680 aa, Alphatorquevirus sp.
TAWWWGRWRRRWRRRRPWRPRLRRRRARRAFPRRRRRRFVSRRWRRPYRRRRRRGRRRRRRRRRHKPTLVLRQWQPDVIRHCKITGRMPLIICGKGSTQFNYITHADDITPRGASYGGNFTNMTFSLEAIYEQFLYHRNRWSASNHDLELCRYKGTTLKLYRHPDVDYIVTYSRTGPFEISHMTYLSTHPLLMLLNKHHIVVPSLKTKPRGRKAIKVRIRPPKLMNNKWYFTRDFCNIGLFQLWATGLELRNPWLRMSTLSPCIGFNVLKNSIYTNLSNLPQHREDRLNIINNTLHPHDITGPNNKKWQYTYTKLMAPIYYSANRASTYDLLREYGLYSPYYLNPTRINLDWMTPYTHVRYNPLVDKGFGNRIYIQWCSEADVSYNRTKSKCLLQDMPLFFMCYGYIDWAIKNTGVSSLARDARICIRCPYTEPQLVGSTEDIGFVPITETFMRGDMPVLAPYIPLSWFCKWYPNIAHQKEVLEAIISCSPFMPRDQGMNGWDITIGYKMDFLWGGSPLPSQPIDDPCQQGTHPIPDPDKHPRLLQVSNPKLLGPRTVFHKWDIRRGQFSKRSIKRVSEYSSDDESLAPGLPSKRNKLDSAFRGENPEQKECYSLLKALEEEETPEEEEPAPQEKAQKEELLHQLQLQRRHQRVLRRGLKLVFTDILRLRQGVHWNPELT

SEQ ID NO: 7 - protein, 197 aa, Alphatorquevirus sp.
TAWWWGRWRRRWRRRRPWRPRLRRRRARRAFPRRRRRRFPIDDPCQQGTHPIPDPDKHPRLLQVSNPKLLGPRTVFHKWDIRRGQFSKRSIKRVSEYSSDDESLAPGLPSKRNKLDSAFRGENPEQKECYSLLKALEEEETPEEEEPAPQEKAQKEELLHQLQLQRRHQRVLRRGLKLVFTDILRLRQGVHWNPELT

SEQ ID NO: 8 - protein, 145 aa, Alphatorquevirus sp.
TAWWWGRWRRRWRRRRPWRPRLRRRRARRAFPRRRRRRFVSHQSETSSTRPSEEKTQSKKNAILSSKHSRKKRPQKKKNQHPKKKPRKRSYSTSSSSRDATSESSDEGSSSSLQTSSDSARESTGTPSSHRAPTLHTRPTFSQYW

SEQ ID NO: 9 - DNA, 3451 bp, Alphatorquevirus sp.
  source 1..3451: mol_type = genomic DNA
aattttgctaaacagactccgaggtgctcttggacactgagtgggcgtacagcaacgaaagtgagtggggccagacttcgccataaggcctttatcttcgggtctacatcataatataaagatgtgcacttccgaatggctgagtttttcacgccattccgcagcggtggagcagcgcagccacgacccccgcgtcccgagggcgggtgccggaggtgagtttacacaccgcagtcaaggggcaattcgggctcgggactggccgggcccgggcaaggctcttaaagcgaaaccatgttcctcggcaggccctaccgccacagaaagcggcaccaggccggcaagaaagggccactgccactgccaaatctgcaacctgcacaggagaaacgggctggtggtccgtccttgatggcctccggacgcaggggatggatgcccccggacctgacggtccaggagagggaggatgcctggtggaccagcttctgcgctagccaccgcagcttttgtagctgcgacgatcctgtgggccatattaatactctcgcccgcgataatagtcctctggcccagactcctactacaacttcaggccaggggccgccgccgccgcctacgcctccgcggacgccggggccgcgccctgggtctgctccggaccaggggggaaggatcagggcctcctggacctaccccctagcccccggaggtcccggtagcacgccatggcctactggtggggccggagacgccggtggcgccgctggaggaggcgccggcgtcctctccgccgccgccggcggtggcggagaaggcgacgctggcccagaaggcgccggtggaggcgaaggagacgacgtgcgagacctgctcgccgctatcgaaggagacgtgggcgcagacgggtaaggagacgccgtcgcccccagaaactagtactgactcagtggaatccccagactgtgagaaagtgtgttattagggggtttctgcccctgttcttctgcggacagggggcctaccacagaaactttacagaccactatgacgatgtgttccccaagggacccagcggaggtgggcacgggagcatggtgttcaacctgtcctttctgtaccaagagtttaagaagcaccacaataagtggtcgcgcagcaacctggactttgacttagtgagatacaagggcacagtgataaagctgtacagacaccaggactttgactacatagtgtggataagcaggacccctcccttccaggagagcctgctcacagtaatgacccaccagcccagcgtcatgctgcaggcaaaaaagtgcataatagtaaagagctacaggacccacccggggggcaaaccctatgtaactgcaaaagttaggccccccagactcctaactgacaagtggtacttccagtcagacttctgcaacgttccgctttttagcctacagtttgcccttgcggaactgcggtttccgatctgctcaccacaaactgacaccaattgcattaacttcctggtgttagatgacatctactacaagtttctagataataagcctaaacagagttcagaccctaatgacgaaaacagaataaaattctggcacggcctatggtccactatgagatatttaaacaccacctacataaacacactgtttccaggcacagacagtctagtggccgccaaagatactgacaatagtgtaaataaataccccagcacagccactaaacagccctacaaagacagtcagtacatgcaaaatatatggaatacatcaaaaatacatgccttatatacgtgggtagcagagacaaactacaaaagactgcaggcctactacacacagacctacggaggctaccagagacaatttttcacaggaaaacagtactgggactacagagtaggcatgtttagtccagccttcctgagtcccagcagactaaatccccagaacccaggggcatacacagaggtctcctacaacccctggacagacgagggcacgggcaacgtagtgtgcctgcagtatctgactaaagagacctcagactacaaaccaggtggtgggagcaagttctgcatagaaggtgtgcctctatgggcagcgctggtgggatacgtagacatgtgtaaaaaagagggcaaggacccgggcatcagactaaactgtctcctgttagtcaagtgtccctatacaaagcctcagctgtatgacaaaaaaaaccccgagaaactgtttgtaccttactcctataactttgggcacggcaagatgccggggggagacaaatacatacccatagagttcaaagacaggtggtacccctgcctgctccaccaagaggagtggatagaggacattgtcaggtcgggacccttcgttccaaaagacatgcccagcagcgtcacctgcatgatgaggtacagctctctttttaactggggcggtaatataatccaagaacaggccgtggaagacccctgtaagaaaggcaccttcgtcgttcccggaaccagtggcatcgctcgcatactacaagtcagcaacccggccaagcagacccccacgacaacctggcactcgtgggactggagacgatccctctttacagagacgggtcttaaaagaatgcgcgaacaacaaccatatgatgaactgtcttatacgggccctaaaaagccaaaactgtcccttcccgcagggcccgccgtccccggtgccgccgtcgcctcctcctggtgggaaacaaaacaggtcacctcgccagacgtcagcgagacggagaccgaagcagaagcccaccaagaggaagagacggagccggaggagggagtccagctccagcagctgtgggagcagcaactcctgcaaaagcgacagctgggagtcgtgttccagcaactcctccgactcagacagggggcggagatccacccgggcctcgtataattcctgggccccagaacccgtacctgcttttcccggagcaggcccctccaaaagtgcctatttttgacccctttggtcagaaaacagagctagagctgtgcggctgcttcgacaggccgcccaggaacaacccctacgaccaccccttctacccctggctgcccaaagagcctccctcctactaccagggctacaaagtgtctttcaaactagggttccacccagacaagcatgtgtgaaccccgccaataaaccactgctgctacactgattcttaggccgtgggagtctcactggtcggtgtctacctcttaaggtcactaagcactccgagcgttagcgaggagtgcgaccctaccccctgggcccacttcttcggagccgcgcgctacgccttcggctgcgcgcggcacctcagacccccgctcgtgctgacacgcttgcgcgtgtcagaccacttcgggctcgcgggggtcggg

SEQ ID NO: 10 - protein, 166 aa, Alphatorquevirus sp.
MASGRRGWMPPDLTVQEREDAWWTSFCASHRSFCSCDDPVGHINTLARDNSPLAQTPTTTSGQGPPPPPTPPRTPGPRPGSAPDQGGRIRASWTYPLAPGGPGSTPWPTGGAGDAGGAAGGGAGVLSAAAGGGGEGDAGPEGAGGGEGDDVRDLLAAIEGDVGADG

SEQ ID NO: 11 - protein, 348 aa, Alphatorquevirus sp.
MASGRRGWMPPDLTVQEREDAWWTSFCASHRSFCSCDDPVGHINTLARDNSPLAQTPTTTSGQGPPPPPTPPRTPGPRPGSAPDQGGRIRASWTYPLAPGGPGSTPWPTGGAGDAGGAAGGGAGVLSAAAGGGGEGDAGPEGAGGGEGDDVRDLLAAIEGDVGADGPWKTPVRKAPSSFPEPVASLAYYKSATRPSRPPRQPGTRGTGDDPSLQRRVLKECANNNHMMNCLIRALKSQNCPFPQGPPSPVPPSPPPGGKQNRSPRQTSARRRPKQKPTKRKRRSRRRESSSSSCGSSNSCKSDSWESCSSNSSDSDRGRRSTRASYNSWAPEPVPAFPGAGPSKSAYF

SEQ ID NO: 12 - protein, 327 aa, Alphatorquevirus sp.
MASGRRGWMPPDLTVQEREDAWWTSFCASHRSFCSCDDPVGHINTLARDNSPLAQTPTTTSGQGPPPPPTPPRTPGPRPGSAPDQGGRIRASWTYPLAPGGPGSTPWPTGGAGDAGGAAGGGAGVLSAAAGGGGEGDAGPEGAGGGEGDDVRDLLAAIEGDVGADGARRPRCRRRLLLVGNKTGHLARRQRDGDRSRSPPRGRDGAGGGSPAPAAVGAATPAKATAGSRVPATPPTQTGGGDPPGPRIIPGPQNPYLLFPEQAPPKVPIFDPFGQKTELELCGCFDRPPRNNPYDHPFYPWLPKEPPSYYQGYKVSFKLGFHPDKHV

SEQ ID NO: 13 - protein, 747 aa, Alphatorquevirus sp.
MAYWWGRRRRWRRWRRRRRPLRRRRRWRRRRRWPRRRRWRRRRRRARPARRYRRRRGRRRVRRRRRPQKLVLTQWNPQTVRKCVIRGFLPLFFCGQGAYHRNFTDHYDDVFPKGPSGGGHGSMVFNLSFLYQEFKKHHNKWSRSNLDFDLVRYKGTVIKLYRHQDFDYIVWISRTPPFQESLLTVMTHQPSVMLQAKKCIIVKSYRTHPGGKPYVTAKVRPPRLLTDKWYFQSDFCNVPLFSLQFALAELRFPICSPQTDTNCINFLVLDDIYYKFLDNKPKQSSDPNDENRIKFWHGLWSTMRYLNTTYINTLFPGTDSLVAAKDTDNSVNKYPSTATKQPYKDSQYMQNIWNTSKIHALYTWVAETNYKRLQAYYTQTYGGYQRQFFTGKQYWDYRVGMFSPAFLSPSRLNPQNPGAYTEVSYNPWTDEGTGNVVCLQYLTKETSDYKPGGGSKFCIEGVPLWAALVGYVDMCKKEGKDPGIRLNCLLLVKCPYTKPQLYDKKNPEKLFVPYSYNFGHGKMPGGDKYIPIEFKDRWYPCLLHQEEWIEDIVRSGPFVPKDMPSSVTCMMRYSSLFNWGGNIIQEQAVEDPCKKGTFVVPGTSGIARILQVSNPAKQTPTTTWHSWDWRRSLFTETGLKRMREQQPYDELSYTGPKKPKLSLPAGPAVPGAAVASSWWETKQVTSPDVSETETEAEAHQEEETEPEEGVQLQQLWEQQLLQKRQLGVVFQQLLRLRQGAEIHPGLV

SEQ ID NO: 14 - protein, 220 aa, Alphatorquevirus sp.
MAYWWGRRRRWRRWRRRRRPLRRRRRWRRRRRWPRRRRWRRRRRRARPARRYRRRRGRRRAVEDPCKKGTFVVPGTSGIARILQVSNPAKQTPTTTWHSWDWRRSLFTETGLKRMREQQPYDELSYTGPKKPKLSLPAGPAVPGAAVASSWWETKQVTSPDVSETETEAEAHQEEETEPEEGVQLQQLWEQQLLQKRQLGVVFQQLLRLRQGAEIHPGLV

SEQ ID NO: 15 - protein, 164 aa, Alphatorquevirus sp.
MAYWWGRRRRWRRWRRRRRPLRRRRRWRRRRRWPRRRRWRRRRRRARPARRYRRRRGRRRGPPSPVPPSPPPGGKQNRSPRQTSARRRPKQKPTKRKRRSRRRESSSSSCGSSNSCKSDSWESCSSNSSDSDRGRRSTRASYNSWAPEPVPAFPGAGPSKSAYF

SEQ ID NO: 16 - DNA, 3753 bp, Alphatorquevirus sp.
  source 1..3753: mol_type = genomic DNA
tgctacgtcactaacccacgtgtcctctacaggccaatcgcagtctatgtcgtgcacttcctgggcatggtctacataattatataaatgcttgcacttccgaatggctgagtttttgctgcccgtccgcggagaggagccacggcaggggatccgaacgtcctgagggcgggtgccggaggtgagtttacacaccgaagtcaaggggcaattcgggctcaggactggccgggctttgggcaaggctcttaaaaatgcacttttctcgaataagcagaaagaaaaggaaagtgctactgctttgcgtgccagcagctaagaaaaaaccaactgctatgagcttctggaaacctccggtacacaatgtcacggggatccaacgcatgtggtatgagtcctttcaccgtggccacgcttctttttgtggttgtgggaatcctatacttcacattactgcacttgctgaaacatatggccatccaacaggcccgagaccttctgggccaccgggagtagaccccaacccccacatccgtagagccaggcctgccccggccgctccggagccctcacaggttgattcgagaccagccctgacatggcatggggatggtggaagcgacggaggcgctggtggttccggaagcggtggacccgtggcagacttcgcagacgatggcctcgatcagctcgtcgccgccctagacgacgaagagtaaggaggcgcagacggtggaggagggggagacgaaaaacaaggacttacagacgcaggagacgctttagacgcaggggacgaaaagcaaaacttataataaaactgtggcaacctgcagtaattaaaagatgcagaataaagggatacataccactgattataagtgggaacggtacctttgccacaaactttaccagtcacataaatgacagaataatgaaaggccccttcgggggaggacacagcactatgaggttcagcctctacattttgtttgaggagcacctcagacacatgaacttctggaccagaagcaacgataacctagagctaaccagatacttgggggcttcagtaaaaatatacaggcacccagaccaagactttatagtaatatacaacagaagaacccctctaggaggcaacatctacacagcaccctctctacacccaggcaatgccattttagcaaaacacaaaatattagtaccaagtttacagacaagaccaaagggtagaaaagcaattagactaagaatagcaccccccacactctttacagacaagtggtactttcaaaaggacatagccgacctcacccttttcaacatcatggcagttgaggctgacttgcggtttccgttctgctcaccacaaactgacaacacttgcatcagcttccaggtccttagttccgtttacaacaactacctcagtattaatacctttaataatgacaactcagactcaaagttaaaagaatttttaaataaagcatttccaacaacaggcacaaaaggaacaagtttaaatgcactaaatacatttagaacagaaggatgcataagtcacccacaactaaaaaaaccaaacccacaaataaacaaaccattagagtcacaatactttgcacctttagatgccctctggggagaccccatatactataatgatctaaatgaaaacaaaagtttgaacgatatcattgagaaaatactaataaaaaacatgattacataccatgcaaaactaagagaatttccaaattcataccaaggaaacaaggccttttgccacctaacaggcatatacagcccaccatacctaaaccaaggcagaatatctccagaaatatttggactgtacacagaaataatttacaacccttacacagacaaaggaactggaaacaaagtatggatggacccactaactaaagagaacaacatatataaagaaggacagagcaaatgcctactgactgacatgcccctatggactttactttttggatatacagactggtgtaaaaaggacactaataactgggacttaccactaaactacagactagtactaatatgcccttatacctttccaaaattgtacaatgaaaaagtaaaagactatgggtacatcccgtactcctacaaattcggagcgggtcagatgccagacggcagcaactacataccctttcagtttagagcaaagtggtaccccacagtactacaccagcaacaggtaatggaggacataagcaggagcgggccctttgcacctaaggtagaaaaaccaagcactcagctggtaatgaagtactgttttaactttaactggggcggtaaccctatcattgaacagattgttaaagaccccagcttccagcccacctatgaaatacccggtaccggtaacatccctagaagaatacaagtcatcgacccgcgggtcctgggaccgcactactcgttccggtcatgggacatgcgcagacacacatttagcagagcaagtattaagagagtgtcagaacaacaagaaacttctgaccttgtattctcaggcccaaaaaagcctcgggtcgacatcccaaaacaagaaacccaagaagaaagctcacattcactccaaagagaatcgagaccgtgggagaccgaggaagaaagcgagacagaagccctctcgcaagagagccaagaggtccccttccaacagcagttgcagcagcagtaccaagagcagctcaagctcagacagggaatcaaagtcctcttcgagcagctcataaggacccaacaaggggtccatgtaaacccatgcctacggtaggtcccaggcagtggctgtttccagagagaaagccagccccagctcctagcagtggagactgggccatggagtttctcgcagcaaaaatatttgataggccagttagaagcaaccttaaagataccccttactacccatatgttaaaaaccaatacaatgtctactttgaccttaaatttgaataaacagcagcttcaaacttgcaaggccgtgggagtttcactggtcggtgtctacctctaaaggtcactaagcactccgagcgtaagcgaggagtgcgaccctcccccctggaacaacttcttcggagtccggcgctacgccttcggctgcgccggacacctcagaccccccctccacccgaaacgcttgcgcgtttcggaccttcggcgtcgggggggtcgggagctttattaaacggactccgaagtgctcttggacactgagggggtgaacagcaacgaaagtgagtggggccagacttcgccataaggcctttatcttcttgccatttgtcagtgtccggggtcgccataggcttcgggctcgtttttaggccttccggactacaaaaatcgccattttggtgacgtcacggccgccatcttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcctctttttcaaaaaaaagcggaagtgccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 17 - protein, 127 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEE

SEQ ID NO: 18 - protein, 268 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEELLKTPASSPPMKYPVPVTSLEEYKSSTRGSWDRTTRSGHGTCADTHLAEQVLRECQNNKKLLTLYSQAQKSLGSTSQNKKPKKKAHIHSKENRDRGRPRKKARQKPSRKRAKRSPSNSSCSSSTKSSSSSDRESKSSSSSS

SEQ ID NO: 19 - protein, 276 aa, Alphatorquevirus sp.
MSFWKPPVHNVTGIQRMWYESFHRGHASFCGCGNPILHITALAETYGHPTGPRPSGPPGVDPNPHIRRARPAPAAPEPSQVDSRPALTWHGDGGSDGGAGGSGSGGPVADFADDGLDQLVAALDDEEPKKASGRHPKTRNPRRKLTFTPKRIETVGDRGRKRDRSPLAREPRGPLPTAVAAAVPRAAQAQTGNQSPLRAAHKDPTRGPCKPMPTVGPRQWLFPERKPAPAPSSGDWAMEFLAAKIFDRPVRSNLKDTPYYPYVKNQYNVYFDLKFE

SEQ ID NO: 20 - protein, 167 aa, Alphatorquevirus sp.
MSFWKPPVHNVTGIQRMWPKKASGRHPKTRNPRRKLTFTPKRIETVGDRGRKRDRSPLAREPRGPLPTAVAAAVPRAAQAQTGNQSPLRAAHKDPTRGPCKPMPTVGPRQWLFPERKPAPAPSSGDWAMEFLAAKIFDRPVRSNLKDTPYYPYVKNQYNVYFDLKFE

SEQ ID NO: 21 - protein, 743 aa, Alphatorquevirus sp.
MAWGWWKRRRRWWFRKRWTRGRLRRRWPRSARRRPRRRRVRRRRRWRRGRRKTRTYRRRRRFRRRGRKAKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSINTFNNDNSDSKLKEFLNKAFPTTGTKGTSLNALNTFRTEGCISHPQLKKPNPQINKPLESQYFAPLDALWGDPIYYNDLNENKSLNDIIEKILIKNMITYHAKLREFPNSYQGNKAFCHLTGIYSPPYLNQGRISPEIFGLYTEIIYNPYTDKGTGNKVWMDPLTKENNIYKEGQSKCLLTDMPLWTLLFGYTDWCKKDTNNWDLPLNYRLVLICPYTFPKLYNEKVKDYGYIPYSYKFGAGQMPDGSNYIPFQFRAKWYPTVLHQQQVMEDISRSGPFAPKVEKPSTQLVMKYCFNFNWGGNPIIEQIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 22 - protein, 194 aa, Alphatorquevirus sp.
MAWGWWKRRRRWWFRKRWTRGRLRRRWPRSARRRPRRRRIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 23 - protein, 113 aa, Alphatorquevirus sp.
MAWGWWKRRRRWWFRKRWTRGRLRRRWPRSARRRPRRRRAQKSLGSTSQNKKPKKKAHIHSKENRDRGRPRKKARQKPSRKRAKRSPSNSSCSSSTKSSSSSDRESKSSSSSS

SEQ ID NO: 24 - DNA, 3878 bp, Alphatorquevirus sp.
  source 1..3878: mol_type = genomic DNA
aaatacgtcactaaccacgtgactcccacaggccaaccacagtctatgtcgtgcacttcctgggcatggtctacgtgataatataaagcggtgcacttccgaatggctgagttttccacgcccgtccgcagcgagatcgcgacgtaggagcgatcgagcgtcccgagggcgggtgccggaggtgagtttacacaccgcagtcaaggggcaattcgggctcgggaggccgggccatgggcaaggctcttaaaaagctatgtttctcggtaaaatctacaggaagaaaaggaaactgcttctgcaggctgtgcgtgctccgcagacgccatcttccatgagccgctgctggtgtccccctcggggtgatgtctcctcccgcgagtctcgatggtacgaggcggttcgaggaagccacgatgctttttgtggctgtagtgatcctattcttcatctttctcgtctggctgcacgttttaaccatcagggacctccgacgccccccacggacgaccgtgcgccgcagaataccccagtgagacgcctgctgcctctccccagctaccccggcgagggtccccaggctagatggcctggtggggatggaggcgccgctggtggcgaccgaagagaaggtggagatggcggcgcgcgcgccgccgaagacgagtaccagcccgaagacctagacgagcttttcggcgctatcgaacaagaacagtaaggaggaggcgaagggggaggcggaggggctaccggcgccgttacagactgagacgctatgccagacgcaggttccgacgcaaaaagatagtactgactcagtggaacccccagactaccagaaaatgtataataaggggcatgatgccagtactgtgggccggcatgggtacggggggcagaaactatgcagtgaggtcagatgactatgtggtgaacaaagggttcgggggctccttcgccacggagaccttctccctgaaggttctctatgaccagtttcaaaggggcttcaacaggtggtcccacactaacgaggacctagacctggcccgctacaggggctgcaggtggactttttacagacataaagacacagactttatagtgtactttacaaacaatcctcccatgaagaccaaccagttctccgcgcccctgacgacccccggcatgctcatgcgcagtaaatacaaagtcctcattcccagcttccagaccagacccaagggtcgcaaaacagtaaccgttaaaataagaccccccaaactatttcaagacaagtggtacacccagcaggacctgtgttcagttcctcttgtccaactgaacgtgaccgcagctgatttcacacatccgttcggctcaccactaactgaaactccttgcgtagagttccaggtgctgggtgacttgtacaatacatgtctcaatatcgaccttccgcaatttagtgaattaggagaaataactagtgcctactcaaaaccaaactcaaataacctaaaagaattatacaaagaattgttcacaaaagccacatcaggacactactggcagacattcataaccaacagcatggtcagagcacacatagatgcagacaaagctaaagaagcacaaagagcatccaccacaccctcatacaacaatgaccccttccccacaatacctgttaaatcagagtttgcacagtggaaaaagaaattcacagacactagagacagcccctttctttttgccacttaccatcccgaagctataaaagacacaattatgaaaatgagagagaacaactttaagctagagacaggacccaatgacaagtatggagactacacagcacagtaccaaggaaacacacacatgctagactactaccttggcttttacagccccatattcctctcagatggaaggtctaacgtagaattcttcactgcctacagagacatagtatacaatcccttcttagacaaggcccagggcaacatggtgtggtttcagtaccacacaaagacagacaacaagtttaaaaaaccagagtgccactgggaaatcaaagacatgcccctgtgggccctcctaaacggatatgtagactacttagagactcaaatacagtatggtgacctcagtaaagaagggaaagtcctcatcaggtgtccctacaccaagccagcactagtagaccccagagacgacactgcaggatatgtagtctacaacagaaactttggcagaggcaagtggatagacggagggggctacatccccctgcacgagaggacaaaatggtacgtgatgctcagataccagacggacgtcttccatgacatagtgacctgtgggccctggcagtacagagacgacaacaaaaacagccagctagtggccaaataccgcttcagctttatatggggaggtaacactgtccactctcaggtcatcagaaacccgtgcaaagacaaccaagtatccggtccccgtcgacagcctagggatatacaagtcgttgacccgcaacgcatcacgccgccgtgggtcctccacagcttcgaccagcgaagaggcctctttactgaaacagctctcaggcgcctgctccaggaaccactacctggcgagtatgctgttagcaccctcaggacacccctcctctttctaccctcagaataccagcgagaagacggcgctgcagaaagcgcctcaggttcaccggccaaaagaccccgtatctggtcagaagagagtcagacggagacgatctcctcggaggagaacccggcggagacgacgagggagctcctccagcgaaagctccgagagcagcgagcactccagttccaactccagcacttcgcggtccaactcgccaagacccaggcgaatctccacgtaaaccccctgttatctttcccgcaatgaataaggtctttctgtttcccccagagggtcccaagcccatcctgggcaaagaggcctggcaggacgagtacgagacctgcagggtctggaacagacctgccagaacccaccacacagacacccccttctatccctgggccccccacaagttccatgtaagcttcaaacttggcttccaataaaattactaggccgtggaactctcactggtcggtgtctacctcttaaggtcactaagcactccgagcgtcagcgaggagtgcgaccctctaccctggtgcaacgccctcggcggccgcgcgctacgccttcggctgcgcgcggcacctcggacccccgctcgtgctgacgcgctcgcgcgcgtcagaccacttcgggctcgcgggggtcgggaattttgctaaacagactccgagttgccattggacactgtagctgtgaatcagtaacgaaagtgagtggggccagacttcgccatagggcctttatcttcttgccattggtccgtgtagggggtcgccataggcttcgacctcccttttaggccttccggactacaaaaatggcggattcagtgacgtcacggccgccattttaagtaggtgccgtccaggactgcagttccgggtcagagtgcatcctcggcggaacctgcacaaaatggcggtcaatatcttccgggtcaaaggtcacacctacgtcataagtcacgtgactgggtcctgctacgtcatatgcggaagtaggccccgccacgtgactcgtcacgtgggcgctgcgtcacggcggccattttgtatcacaaaatggcggacttccttcctcttttttaaaaataacggcccagcggcggcgcgcgcgcttcgcgcgcgcgccggggggctccgcccccccccgcgcatgcgcggggcccccccccgcggggggctccgccccccggtccccccccg

SEQ ID NO: 25 - protein, 128 aa, Alphatorquevirus sp.
MSRCWCPPRGDVSSRESRWYEAVRGSHDAFCGCSDPILHLSRLAARFNHQGPPTPPTDDRAPQNTPVRRLLPLPSYPGEGPQARWPGGDGGAAGGDRREGGDGGARAAEDEYQPEDLDELFGAIEQEQ

SEQ ID NO: 26 - protein, 279 aa, Alphatorquevirus sp.
MSRCWCPPRGDVSSRESRWYEAVRGSHDAFCGCSDPILHLSRLAARFNHQGPPTPPTDDRAPQNTPVRRLLPLPSYPGEGPQARWPGGDGGAAGGDRREGGDGGARAAEDEYQPEDLDELFGAIEQEQSSETRAKTTKYPVPVDSLGIYKSLTRNASRRRGSSTASTSEEASLLKQLSGACSRNHYLASMLLAPSGHPSSFYPQNTSEKTALQKAPQVHRPKDPVSGQKRVRRRRSPRRRTRRRRRGSSSSESSESSEHSSSNSSTSRSNSPRPRRIST

SEQ ID NO: 27 - protein, 272 aa, Alphatorquevirus sp.
MSRCWCPPRGDVSSRESRWYEAVRGSHDAFCGCSDPILHLSRLAARFNHQGPPTPPTDDRAPQNTPVRRLLPLPSYPGEGPQARWPGGDGGAAGGDRREGGDGGARAAEDEYQPEDLDELFGAIEQEQIPARRRRCRKRLRFTGQKTPYLVRRESDGDDLLGGEPGGDDEGAPPAKAPRAASTPVPTPALRGPTRQDPGESPRKPPVIFPAMNKVFLFPPEGPKPILGKEAWQDEYETCRVWNRPARTHHTDTPFYPWAPHKFHVSFKLGFQ

SEQ ID NO: 28 - protein, 780 aa, Alphatorquevirus sp.
MAWWGWRRRWWRPKRRWRWRRARRRRRVPARRPRRAFRRYRTRTVRRRRRGRRRGYRRRYRLRRYARRRFRRKKIVLTQWNPQTTRKCIIRGMMPVLWAGMGTGGRNYAVRSDDYVVNKGFGGSFATETFSLKVLYDQFQRGFNRWSHTNEDLDLARYRGCRWTFYRHKDTDFIVYFTNNPPMKTNQFSAPLTTPGMLMRSKYKVLIPSFQTRPKGRKTVTVKIRPPKLFQDKWYTQQDLCSVPLVQLNVTAADFTHPFGSPLTETPCVEFQVLGDLYNTCLNIDLPQFSELGEITSAYSKPNSNNLKELYKELFTKATSGHYWQTFITNSMVRAHIDADKAKEAQRASTTPSYNNDPFPTIPVKSEFAQWKKKFTDTRDSPFLFATYHPEAIKDTIMKMRENNFKLETGPNDKYGDYTAQYQGNTHMLDYYLGFYSPIFLSDGRSNVEFFTAYRDIVYNPFLDKAQGNMVWFQYHTKTDNKFKKPECHWEIKDMPLWALLNGYVDYLETQIQYGDLSKEGKVLIRCPYTKPALVDPRDDTAGYVVYNRNFGRGKWIDGGGYIPLHERTKWYVMLRYQTDVFHDIVTCGPWQYRDDNKNSQLVAKYRFSFIWGGNTVHSQVIRNPCKDNQVSGPRRQPRDIQVVDPQRITPPWVLHSFDQRRGLFTETALRRLLQEPLPGEYAVSTLRTPLLFLPSEYQREDGAAESASGSPAKRPRIWSEESQTETISSEENPAETTRELLQRKLREQRALQFQLQHFAVQLAKTQANLHVNPLLSFPQ

SEQ ID NO: 29 - protein, 204 aa, Alphatorquevirus sp.
MAWWGWRRRWWRPKRRWRWRRARRRRRVPARRPRRAFRRYRTRTVIRNPCKDNQVSGPRRQPRDIQVVDPQRITPPWVLHSFDQRRGLFTETALRRLLQEPLPGEYAVSTLRTPLLFLPSEYQREDGAAESASGSPAKRPRIWSEESQTETISSEENPAETTRELLQRKLREQRALQFQLQHFAVQLAKTQANLHVNPLLSFPQ

SEQ ID NO: 30 - protein, 119 aa, Alphatorquevirus sp.
MAWWGWRRRWWRPKRRWRWRRARRRRRVPARRPRRAFRRYRTRTNTSEKTALQKAPQVHRPKDPVSGQKRVRRRRSPRRRTRRRRRGSSSSESSESSEHSSSNSSTSRSNSPRPRRIST

SEQ ID NO: 31 - DNA, 3818 bp, Alphatorquevirus sp.
  source 1..3818: mol_type = genomic DNA
aagtccgccactaaccacgtgactcccgcaggccaacccagtactatgtcgtccacttcctgggacgagtctacgtcctgatataagtaagtgcacttccgaatggctgagttttccacgcccgtccgcagcgagaacgccacggaggggagtccgcgcgtcccgagggcgggtgccggaggtgagtttacacaccgcagtcaaggggcaattcgggctcgggactggccgggccccgggcaaggctcttaaaaaatgcactttcgcagagtgcgagcgaaaaggaaactgctactgcaagctgtgcgagctccaccgaaggcacctgccatgagcttcaccacacctactattaatgccgggatccgagagcagcaatggttcgagtccacccttagatcccaccactcgttctgtggctgtggtgatcccgtgcttcattttactaaccttgctactcgctttaactatctgcctgctacctcttcgcctctggaccctcccggcccagcgccgcgaggccgcccggcgctccgccgcctcccggcactcccttcagcccccgcgaccccttctagagaactagcatggcctactggttcagaaggtggggctggaggccgaggcgccggtggagaaggtggcgccgccgtcgaaggagactaccgagaagaagaactagacgagctgttcgcggccttggaagaagacgcaaaccaagggtaaggaggcgccgcagaactcgcagacgtacctacagacgggggtggagacgcaggaggtacataagacgggggcgacgcaaaaagaaactcatactgactcagtggaacccggcaatagttaagaggtgcaacattaagggcggacttccaataattatatgcggagagcccagggcagcctttaactatggctaccacatggaggactacactcctcaacctttccccttcggagggggaatgagcacagtgactttctctctgaaagccttgtatgaccagtacctaaaacaccaaaacaggtggactttctcaaacgaccagctagacctcgccagatacaggggctgtaaactaaggttctacagaagccccgtctgtgactttatagtacactacaacctaatacctccactaaaaatgaaccagttcacaagtcccaacacgcacccgggactactcatgctcagcaaacacaagataataattcccagctttcaaacaagacctgggggcagacgctttgttaaaataagacttaatccccccaaactatttgaagacaagtggtacactcagcaagacctgtgcaaggttccgctcgttagtattacagcaactgcggctgacttgcggtatccgttctgctcaccacaaacgaacaacccttgcaccaccttccaggtactgcgcaagaactacaatacagttataggaacttccgtaaaagaccaagagtccacacaagactttgaaaattggctttataaaacagactcacactatcaaacatttgccacagaggctcaactaggcagaattcctgcatttaatcctgatggcactaaaaacactaaacagcagtcgtggcaagataactggagcaaaaaaaattcaccatggacaggtaactcaggtacatacccacaaacaaccagtgaaatgtacaaaattccatatgacagtaacttcggctttcccacatacagagcccaaaaagactacattttagaaagaagacagtgcaactttaactatgaagttaataatccagttagcaaaaaagtatggccacaacctagtacaacaacacccacagtagactactatgaataccactgtggatggttcagcaacatattcataggccccaacagatacaacctacagtttcaaacagcatatgtagacaccacatacaacccactaatggacaagggcaaaggcaacaaaatatggtttcaatatctgtctaaaaagggcacagactacaatgaaaaacaatgctactgcaccctagaagacatgcccctatgggcaatatgctttggatacactgactatgtagagactcaactaggacccaatgtggaccatgaaacagcaggcttaataattatgatctgtccatacactcaaccacctatgtatgacaaaaacagacctaactggggatacgtagtctatgacacaaactttggcaatggaaaaatgccctcaggaagtggccaagtcccagtatactggcaatgccgatggaggcccatgctgtggttccaacaacaagtactcaatgacatctcaaagactggaccgtacgcctacagagacgaatataaaaatgtacaactgactctctactacaactttatttttaactgggggggcgacatgtattacccacaggtcgttaaaaacccctgtggagactccggaatcgttcccggttccggtagattcactcgagaagtacaagtcgttagcccgctttccatgggaccggcctacatcttccactacttcgactccagacgcgggttctttagtgaaaaagctcttaaaagaatgcaacaacaacaagaatttgatgaatcttttacattcaaacctaagagacccaaactttctacagcagccgcagaaatcctccagctcgaagaagactcgacttcaggggaaggaaaatcgccactacagcaagaagagaaagaagtcgaagtcctccaaacgccgacagtacagctccagctccagcgaaacatccaggagcagctcgcaatcaagcagcagctccaattcctcttgctccaactcctcaaaacccaatccaatttgcatttaaacccacaatttttaagcccttcataaaatatgacatgtttggggacccccttcctcaccccccaacagccgaagagtgggaaacagagtaccagtgctgtaaggcctttaacagaccacctagaaccaacctaaaagacacccccttctacccctgggtacctaaacctaaacctcaattccgtgtatcttttaaacttggttttcaataaacaaggccgtgggagtttcacttgtcggtgtcaacctcttaaggtcactaagcactccgagcgtaagcgaggagtgcgaccctcccccctggggcaactccctcgaagtccggcgctacgcgcttcgcgctgcgccggacatctcggaccccccctccacccgaaacgcttgcgcgtttcggaccttcggcgtcgggggggtcgggggctttactaaacagactccgaggtgccattggacactgaggggatgaacagcaacgaaagtgagtggggccagacttcgccataaggcctttatcttcttgccatttgtcagtatagagggtcgccataggcttcggcctccattttaacctctaaaaactaccaaaatggccgttccagtgacgtcacagccgccattttaagtagctgacgtcaaggattgacgtgaaggttaaaggtcatcctcggcggaagctacacaaaatggtggacaacatcttccgggtcaaaggtcgtgcacacgtcataagtcacgtggtggggacccgctgtaacccggaagtaggccccgtcacgtgatttgtcacgtgtgtacacgtcacaaccgccattttgttttacaaaatggctgacttccttcctcttttttaaaaaaaacggccgtgcggcggcgcgcgcgcttcgcgcgcgcgccgggggctgccgcccccccccgcgcatgcgcgcggggcccccccccgcggggggctccgccccccggcccccccccccg

SEQ ID NO: 32 - protein, 131 aa, Alphatorquevirus sp.
MSFTTPTINAGIREQQWFESTLRSHHSFCGCGDPVLHFTNLATRFNYLPATSSPLDPPGPAPRGRPALRRLPALPSAPATPSRELAWPTGSEGGAGGRGAGGEGGAAVEGDYREEELDELFAALEEDANQG

SEQ ID NO: 33 - protein, 275 aa, Alphatorquevirus sp.
MSFTTPTINAGIREQQWFESTLRSHHSFCGCGDPVLHFTNLATRFNYLPATSSPLDPPGPAPRGRPALRRLPALPSAPATPSRELAWPTGSEGGAGGRGAGGEGGAAVEGDYREEELDELFAALEEDANQGSLKTPVETPESFPVPVDSLEKYKSLARFPWDRPTSSTTSTPDAGSLVKKLLKECNNNKNLMNLLHSNLRDPNFLQQPQKSSSSKKTRLQGKENRHYSKKRKKSKSSKRRQYSSSSSETSRSSSQSSSSSNSSCSNSSKPNPICI

SEQ ID NO: 34 - protein, 270 aa, Alphatorquevirus sp.
MSFTTPTINAGIREQQWFESTLRSHHSFCGCGDPVLHFTNLATRFNYLPATSSPLDPPGPAPRGRPALRRLPALPSAPATPSRELAWPTGSEGGAGGRGAGGEGGAAVEGDYREEELDELFAALEEDANQGSRRNPPARRRLDFRGRKIATTARRERSRSPPNADSTAPAPAKHPGAARNQAAAPIPLAPTPQNPIQFAFKPTIFKPFIKYDMFGDPLPHPPTAEEWETEYQCCKAFNRPPRTNLKDTPFYPWVPKPKPQFRVSFKLGFQ

SEQ ID NO: 35 - protein, 156 aa, Alphatorquevirus sp.
MSFTTPTINAGIREQQCSRRNPPARRRLDFRGRKIATTARRERSRSPPNADSTAPAPAKHPGAARNQAAAPIPLAPTPQNPIQFAFKPTIFKPFIKYDMFGDPLPHPPTAEEWETEYQCCKAFNRPPRTNLKDTPFYPWVPKPKPQFRVSFKLGFQ

SEQ ID NO: 36 - protein, 761 aa, Alphatorquevirus sp.
MAYWFRRWGWRPRRRWRRWRRRRRRLPRRRTRRAVRGLGRRRKPRVRRRRRTRRRTYRRGWRRRRYIRRGRRKKKLILTQWNPAIVKRCNIKGGLPIIICGEPRAAFNYGYHMEDYTPQPFPFGGGMSTVTFSLKALYDQYLKHQNRWTFSNDQLDLARYRGCKLRFYRSPVCDFIVHYNLIPPLKMNQFTSPNTHPGLLMLSKHKIIIPSFQTRPGGRRFVKIRLNPPKLFEDKWYTQQDLCKVPLVSITATAADLRYPFCSPQTNNPCTTFQVLRKNYNTVIGTSVKDQESTQDFENWLYKTDSHYQTFATEAQLGRIPAFNPDGTKNTKQQSWQDNWSKKNSPWTGNSGTYPQTTSEMYKIPYDSNFGFPTYRAQKDYILERRQCNFNYEVNNPVSKKVWPQPSTTTPTVDYYEYHCGWFSNIFIGPNRYNLQFQTAYVDTTYNPLMDKGKGNKIWFQYLSKKGTDYNEKQCYCTLEDMPLWAICFGYTDYVETQLGPNVDHETAGLIIMICPYTQPPMYDKNRPNWGYVVYDTNFGNGKMPSGSGQVPVYWQCRWRPMLWFQQQVLNDISKTGPYAYRDEYKNVQLTLYYNFIFNWGGDMYYPQVVKNPCGDSGIVPGSGRFTREVQVVSPLSMGPAYIFHYFDSRRGFFSEKALKRMQQQQEFDESFTFKPKRPKLSTAAAEILQLEEDSTSGEGKSPLQQEEKEVEVLQTPTVQLQLQRNIQEQLAIKQQLQFLLLQLLKTQSNLHLNPQFLSPS

SEQ ID NO: 37 - protein, 198 aa, Alphatorquevirus sp.
MAYWFRRWGWRPRRRWRRWRRRRRRLPRRRTRRAVRGLGRRRKPRVVKNPCGDSGIVPGSGRFTREVQVVSPLSMGPAYIFHYFDSRRGFFSEKALKRMQQQQEFDESFTFKPKRPKLSTAAAEILQLEEDSTSGEGKSPLQQEEKEVEVLQTPTVQLQLQRNIQEQLAIKQQLQFLLLQLLKTQSNLHLNPQFLSPS

SEQ ID NO: 38 - protein, 114 aa, Alphatorquevirus sp.
MAYWFRRWGWRPRRRWRRWRRRRRRLPRRRTRRAVRGLGRRRKPRQPQKSSSSKKTRLQGKENRHYSKKRKKSKSSKRRQYSSSSSETSRSSSQSSSSSNSSCSNSSKPNPICI

SEQ ID NO: 39 - DNA, 3794 bp, Alphatorquevirus sp.
  source 1..3794: mol_type = genomic DNA
cccgaagtccgtcactaaccacgtgactcctgtcgcccaatcagagtgtatgtcgtgcatttcctgggcatggtctacatcctgatataactaagtgcacttccgaatggctgagttttccacgcccgtccgcagcgagggagcgacggaggagctcccgagcgtcccgagggcgggtgccggaggtgagtttacacaccgcagtcaaggggcaattcgggctcgggactggccgggctatgggcaaggctcttagggtcttcattcttaatatgtttcttggcagagtttaccgccacaagaaaaggaaagtgctactgtccacactgcgagctccacaggcgtctcgcagggctatgagttggcgacccccggtacacgatgcacccggcatcgagcgcaattggtacgaggcctgtttcagagcccacgctggagcttgtggctgtggcaattttattatgcaccttaatcttttggctgggcgttatggttttactccggggtcagcgccgccaggtggtcctcctccgggcaccccgcagataaggagagccaggcctagtcccgccgcaccagagcagcccgctgccctaccatggcatggggatggtggagatggcggcgccgctggcccgccagacgctggaggagacgccgtcgccggcgccccgtacggagaacaagagctcgccgacctgctcgacgctatagaagacgacgaacagtaagaaccaggcgaaggcggtgggggcgcagacggtacagacggggctggagacgcaggacttatgtgagaaaggggcgacacagaaaaaagaaaaagagactgatactgagacagtggcaaccagccacaagacgcagatgtaccataactgggtacctgcccatagtgttctgcggccacactaggggcaataaaaactatgcactacactctgacgactacaccccccaaggacaaccatttggaggggctctaagcactacctcattctctttaaaagtactatttgaccagcatcagagaggactaaacaagtggtcttttccaaacgaccaactagacctcgccagatatagaggctgcaaatttatattttatagaacaaaacaaactgactgggtgggccagtatgacatatcagaaccctacaagctagacaaatacagctgccccaactatcaccctggaaacatgattaaggcaaagcacaaatttttaataccaagctatgacactaatcctagaggcagacaaaaaattatagttaaaattccccccccagacctctttgtagacaagtggtacactcaagaggatctgtgttccgttaatcttgtgtcacttgcggtttctgcggcttcctttctccacccattcggctcaccacaaactgacaacccttgctacaccttccaggtgttgaaagagttctactatcaggcaataggcttctctgcaagcacacaagcaatgacatcagtattagacacgctatacacacaaaacagttattgggaatctaatctaactcagttttatgtacttaatgcaaaaaaaggcagtgatacaacacagcctttaactagcaatatgccaactcgtgaagagtttatggcaaaaaaaaataccaattacaactggtatacatacaaggccgcgtcagtaaaaaataaactacatcaaatgagacaaacctattttgaggagttaacctctaaggggccacaaacaacaaaaagtgaggaaggctacagtcagcactggaccaccccctccacaaacgcctacgaatatcacttaggaatgtttagtgcaatatttctagccccagacaggccagtacctagatttccatgcgcctaccaagatgtaacttacaaccccttaatggacaaaggggtgggaaaccacatttggtttcagtacaacacaaaggcagacactcagctaatagtcacaggagggtcctgcaaagcacacatacaagacataccactgtgggcggccttctatggatacagtgactttatagagtcagaactaggcccctttgtagatgcagagacggtaggcttagtgtgtgtaatatgcccttatacaaaaccccccatgtacaacaagacaaaccccgccatgggctacgtgttctatgacagaaactttggtgacggaaaatggactgacggacggggcaaaatagagccctactggcaagttaggtggaggcccgaaatgcttttccaagaaactgtaatggcagacctagttcagactgggccctttagctacaaagacgaacttaaaaacagcaccctagtgtgcaagtacaaattctatttcacctggggaggtaacatgatgttccaacagacgatcaaaaacccgtgcaagacggacggacaacccaccgactccagtagacaccctagaggaatacaagtggcggacccggaacaaatgggaccccgctgggtgttccactcctttgactggcgaaggggctatcttagcgagaaagctctcaaacgcctgcaagaaaaacctcttgactatgacgaatattttacacaaccaaaaagacctagaatctttcctccaacagaatcagcagagggagagttccgagagcccgaaaaaggctcgtattcagaggaagaaaggtcgcaagcctctgccgaagagcagacgcaggaggcgacagtactcctcctcaagcgacgactcagagagcaacagcagctccagcagcagctccaattcctcacccgagaaatgttcaaaacgcaagcgggtctccacctaaaccctatgttattaaaccagcgataaaccaagtgtacctgtttccagagagggccccaaaaccccctcctagcagccaagactggcagcaggagtacgaggcctgcgcagcctgggacaggccccctagatacaatctgtcctctcctcctttctaccccagctgcccttcaaaattctgtgtaaaattcagccttggctttaaataaatggcaactttactgtgcaaggccgtgggagtttcactggtcggtgtctacctctaaaggtcactaagcactccgagcgttagcgaggagtgcgacccttccccctgactcaacttcttcggagccgcgcgctacgccttcggctgcgcgcggcacctcagacccccgctcgtgctgacacgctcgcgcgtgtcagaccacttcgggctcgcgggggtcgggaattttgctaaacagactccgagttgctcttggacactgagggggcatatcagtaacgaaagtgagtggggccagacttcgccataaggcctttatcttcttgccattggatagtatcgagggttgccataggcttcgacctccattttaggccttccggactacaaaaatggccgttttagtgacgtcacggccgccattttaagtaaggcggaagcagctcggcgtacacaaaatggcggcggagcacttccggcttgcccaaaatggtgggcaacttcttccgggtcaaaggtcacagctacgtcacaagtcacgtggggagggttggcgtttaacccggaagccaatcctcttacgtggcctgtcacgtgacttgtacgtcacgaccaccattttgttttacaaaatggccgacttccttcctcttttttaaaaataacggttcggcggcggcgcgcgcgctacgcgcgcgcgccggggggctgccgccccccccccgcgcatgcgcggggcccccccccgcggggggctccgccccccggcccccc

SEQ ID NO: 40 - protein, 124 aa, Alphatorquevirus sp.
MSWRPPVHDAPGIERNWYEACFRAHAGACGCGNFIMHLNLLAGRYGFTPGSAPPGGPPPGTPQIRRARPSPAAPEQPAALPWHGDGGDGGAAGPPDAGGDAVAGAPYGEQELADLLDAIEDDEQ

SEQ ID NO: 41 - protein, 267 aa, Alphatorquevirus sp.
MSWRPPVHDAPGIERNWYEACFRAHAGACGCGNFIMHLNLLAGRYGFTPGSAPPGGPPPGTPQIRRARPSPAAPEQPAALPWHGDGGDGGAAGPPDAGGDAVAGAPYGEQELADLLDAIEDDEQRSKTRARRTDNPPTPVDTLEEYKWRTRNKWDPAGCSTPLTGEGAILARKLSNACKKNLLTMTNILHNQKDLESFLQQNQQRESSESPKKARIQRKKGRKPLPKSRRRRRQYSSSSDDSESNSSSSSSSNSSPEKCSKRKRVST

SEQ ID NO: 42 - protein, 257 aa, Alphatorquevirus sp.
MSWRPPVHDAPGIERNWYEACFRAHAGACGCGNFIMHLNLLAGRYGFTPGSAPPGGPPPGTPQIRRARPSPAAPEQPAALPWHGDGGDGGAAGPPDAGGDAVAGAPYGEQELADLLDAIEDDEHRGRVPRARKRLVFRGRKVASLCRRADAGGDSTPPQATTQRATAAPAAAPIPHPRNVQNASGSPPKPYVIKPAINQVYLFPERAPKPPPSSQDWQQEYEACAAWDRPPRYNLSSPPFYPSCPSKFCVKFSLGFK

SEQ ID NO: 43 - protein, 150 aa, Alphatorquevirus sp.
MSWRPPVHDAPGIERNCRGRVPRARKRLVFRGRKVASLCRRADAGGDSTPPQATTQRATAAPAAAPIPHPRNVQNASGSPPKPYVIKPAINQVYLFPERAPKPPPSSQDWQQEYEACAAWDRPPRYNLSSPPFYPSCPSKFCVKFSLGFK

SEQ ID NO: 44 - protein, 746 aa, Alphatorquevirus sp.
MAWGWWRWRRRWPARRWRRRRRRRPVRRTRARRPARRYRRRRTVRTRRRRWGRRRYRRGWRRRTYVRKGRHRKKKKRLILRQWQPATRRRCTITGYLPIVFCGHTRGNKNYALHSDDYTPQGQPFGGALSTTSFSLKVLFDQHQRGLNKWSFPNDQLDLARYRGCKFIFYRTKQTDWVGQYDISEPYKLDKYSCPNYHPGNMIKAKHKFLIPSYDTNPRGRQKIIVKIPPPDLFVDKWYTQEDLCSVNLVSLAVSAASFLHPFGSPQTDNPCYTFQVLKEFYYQAIGFSASTQAMTSVLDTLYTQNSYWESNLTQFYVLNAKKGSDTTQPLTSNMPTREEFMAKKNTNYNWYTYKAASVKNKLHQMRQTYFEELTSKGPQTTKSEEGYSQHWTTPSTNAYEYHLGMFSAIFLAPDRPVPRFPCAYQDVTYNPLMDKGVGNHIWFQYNTKADTQLIVTGGSCKAHIQDIPLWAAFYGYSDFIESELGPFVDAETVGLVCVICPYTKPPMYNKTNPAMGYVFYDRNFGDGKWTDGRGKIEPYWQVRWRPEMLFQETVMADLVQTGPFSYKDELKNSTLVCKYKFYFTWGGNMMFQQTIKNPCKTDGQPTDSSRHPRGIQVADPEQMGPRWVFHSFDWRRGYLSEKALKRLQEKPLDYDEYFTQPKRPRIFPPTESAEGEFREPEKGSYSEEERSQASAEEQTQEATVLLLKRRLREQQQLQQQLQFLTREMFKTQAGLHLNPMLLNQR

SEQ ID NO: 45 - protein, 195 aa, Alphatorquevirus sp.
MAWGWWRWRRRWPARRWRRRRRRRPVRRTRARRPARRYRRRRTTIKNPCKTDGQPTDSSRHPRGIQVADPEQMGPRWVFHSFDWRRGYLSEKALKRLQEKPLDYDEYFTQPKRPRIFPPTESAEGEFREPEKGSYSEEERSQASAEEQTQEATVLLLKRRLREQQQLQQQLQFLTREMFKTQAGLHLNPMLLNQR

SEQ ID NO: 46 - protein, 107 aa, Alphatorquevirus sp.
MAWGWWRWRRRWPARRWRRRRRRRPVRRTRARRPARRYRRRRTQRESSESPKKARIQRKKGRKPLPKSRRRRRQYSSSSDDSESNSSSSSSSNSSPEKCSKRKRVST

SEQ ID NO: 47 - DNA, 3866 bp, Alphatorquevirus sp.
  source 1..3866: mol_type = genomic DNA
aagtccgtcactaaccacgtgactcccgcaggccaatcagagtctatgtcgtgcacttcctgggcatggtctacgttctcatataactaactgcacttccgaatggctgagttttccacgcccgtccgcagcggcagcaccacggagggtgatccccgcgtcccgagggcgggtgccgaaggtgagtttacacaccgcagtcaaggggcaattcgggctcgggactggccgggctatgggcaaggctcttagggctttcattgttaaaaatgtttctcggcaggccttacaggagaaagaaaagggcgctgtcactgcctggcgtgcgagctgcacaggcgaaacaacctggtgatatgagctggagccgtccagtacataatgccgccgggatcgaaaggcagtggttcgaatccacctttagatcccacgctagttgctgtggctgcggcaattttgttaatcatattaatgtactggctgctcgctacggctttactggggggccgacgccgccaggtggtcctgggccgcgtccacaactgaggcccgcgcttcccgcgccggaccccgacccccaggcgcccaaccgtgagccatggcgtggagctggtggtggcaacgatggagaaggcgccgctggaaacccaggaggcgccgctggagacgtctacgatggagaagacctagacgcgctgttcgccgccgtcgtcgaggacgtagagtaaggaggcggaggtgggcgcgtagacgggggcgacgcagacggtacgccaccagacgaaagagacgttataggggtcgccgctttaaaaagaaactagtactgactcagtggcaccctaataccatgagacgctgcttaatcaagggcatagtccccctggtaatatgcggccacaccaggtggaactacaactacgccctccatagcaaggactacacagaggagggtcgctaccctcacgggggggccctcagcaccactacgtggtcccttaaggtgctgtatgacgagcacctcaaacaccacgacttctggggctatcccaacaaccagctagacctggccaggtacaagggggccaagttcaccttctacagacacaaaaagactgactttataatattctttaacagaaagcctccctttaagctaaacaagtacagctgtgcctcctatcacccaggcatgctgatgcagcagagacacaagatcctgctacccagctacgaaactaaacccaagggcaggccaaagataacagttagaataaagccccccactctgttagaggacaagtggtacacccagcaggacctgtgcgacgttaacctgttgcaacttgtggtcactgcggctgactttcgacatccactctgctcaccacaaacgaacactccaaccacaaccttccaggtgttgaaagacatctattatgacactatgagcatatctgaacccacagactcctacactagtgttaacaataaaagtacaacacaaacttttactaactactcaaacaccttagaaaacattctgtacacacgagcctcctactggaactcgttccacgccactgaatacctaaaccccaacatcatatacaaaaacggtgaaaaactattcaaagaacatgaagacttaataacctggatgacccaaactaacaataccgggtttctaactaaaaacaacacagcttttggcaacaacagctacaggcccaatgcagacaaaattaaaaaagccagaaagacatactggaacgccctaataggcaccaacgacctggccactaatataggccaggccagagcagaaaggttcgagtaccacctaggctggtactcccccatatttctcagcagacacaggagcaacatgaactttgccagggcctaccaagacgtcacatacaaccccaactgtgacaggggagttaacaacagggtgtgggttcagcctctaactaaacccaccacagagttcgacgagaaaaggtgtaagtgcgtagtgcagcacctgcctctgtgggcggctctgtactgctaccaagactttgtagaggaggagctggggtcctcctcagagatattaaattcatgcctactggtattacagtgcccttacacctttcccccaatgtatgacaaaaagctaccagacaagggattcgtgttttatgactccctttttggagacggcaaaatgtctgacggacgcggacaggtggacattttctggcaacagcgatggtaccctcgcttagccactcagatgcaagtcatgcacgacatcaccatgacgggccccttctcctaccgagacgagctagttagcacccaactgactgccaagtacacctttgactttatgtggggcggaaatatgatctccacacagatcatcaagaacccctgcaaagacagtggactggaacccgcctaccccggtagacagcgtcgcgacttacaaattgttgacccatactccatgggcccccaattctcgttccacaactgggactacagacatggcctttttggccaagacgctatcgacagagtgtctaaacaaccaaaagatgatgcagactatcctaacccatacaaaaggcctagatattttccacccacagaccaagccgcccaagagcaagaaaaagacttcagtttcctcaaaacagcaccgtcgaactcagaagagagcgatcaagaagtcctccaagaaacgcaagtactccgattccagccagagcagcacaagcaactccacctgcagctcgcagagcggcagcgaatcggagagcaactccgatacctactccaacagatgttcaaaactcaggccaatctccacctaaacccatatacatttacccagctgtaaagcaggtgtttatgtttgaccccccgggccctaaggctatctcgggcgccaaggcctgggaggacgagttcctcaccgcaaaagtgtggaaccgcccggtacgcaagtactactcagacaccccctactacccctgggcccccaaaccccagtactctgtcagtttcaaactcggctggaaataaaaaaagcctgctccactgtactaggccgtgggagtttcactcgtcggtgtctacctcttaaggtcaccaagcactccgagcgtcagcgaggagtgcgacccttgggggtgggtgcaacgccctcggcggccgcgcgctacgccttcggctgcgcgcggcacctcggacccccgctcgtgctgacgcgcttgcgcgcgtcagaccacttcgggctcgcgggggtcggaaattttgctaaacagactccgagttgccattggacactggagccgtgaatcagtaacgaaagtgagtggggccagacttcgccataaggcctttatctttttgccatttgtccgtggggaagggtcgctgcaagcgcggaccccgttttcaccccttccggactacaaaaatagcgcattagtgacgtcacggccgccattttaagtaaggcggaagcaactccactttctcacaaaatggcggcggagcacttccggcttgcccaaaatggccgccaaaaacatccgggtcaaagttcgccgctacgtcataagtcacgtgactggggaggtacttaaacacggaagtatcctcaaccacgtaactggtcacgtggtgcgcacgtcacggcaaccattttgttttacaaaatggcgcatttccttcctcttttttaaaaattaaccgttggcggcggcgcgcgcgctacgcgcgcgcgccggggagctctgcccccccccgcgcatgcgcgcgggtcccccccccgcggggggctccgccccccggtcccccccccg

SEQ ID NO: 48 - protein, 123 aa, Alphatorquevirus sp.
MSWSRPVHNAAGIERQWFESTFRSHASCCGCGNFVNHINVLAARYGFTGGPTPPGGPGPRPQLRPALPAPDPDPQAPNREPWRGAGGGNDGEGAAGNPGGAAGDVYDGEDLDALFAAVVEDVE

SEQ ID NO: 49 - protein, 275 aa, Alphatorquevirus sp.
MSWSRPVHNAAGIERQWFESTFRSHASCCGCGNFVNHINVLAARYGFTGGPTPPGGPGPRPQLRPALPAPDPDPQAPNREPWRGAGGGNDGEGAAGNPGGAAGDVYDGEDLDALFAAVVEDVESSRTPAKTVDWNPPTPVDSVATYKLLTHTPWAPNSRSTTGTTDMAFLAKTLSTECLNNQKMMQTILTHTKGLDIFHPQTKPPKSKKKTSVSSKQHRRTQKRAIKKSSKKRKYSDSSQSSTSNSTCSSQSGSESESNSDTYSNRCSKLRPIST

SEQ ID NO: 50 - protein, 267 aa, Alphatorquevirus sp.
MSWSRPVHNAAGIERQWFESTFRSHASCCGCGNFVNHINVLAARYGFTGGPTPPGGPGPRPQLRPALPAPDPDPQAPNREPWRGAGGGNDGEGAAGNPGGAAGDVYDGEDLDALFAAVVEDVEPSRPRARKRLQFPQNSTVELRRERSRSPPRNASTPIPARAAQATPPAARRAAANRRATPIPTPTDVQNSGQSPPKPIYIYPAVKQVFMFDPPGPKAISGAKAWEDEFLTAKVWNRPVRKYYSDTPYYPWAPKPQYSVSFKLGWK

SEQ ID NO: 51 - protein, 765 aa, Alphatorquevirus sp.
MAWSWWWQRWRRRRWKPRRRRWRRLRWRRPRRAVRRRRRGRRVRRRRWARRRGRRRRYATRRKRRYRGRRFKKKLVLTQWHPNTMRRCLIKGIVPLVICGHTRWNYNYALHSKDYTEEGRYPHGGALSTTTWSLKVLYDEHLKHHDFWGYPNNQLDLARYKGAKFTFYRHKKTDFIIFFNRKPPFKLNKYSCASYHPGMLMQQRHKILLPSYETKPKGRPKITVRIKPPTLLEDKWYTQQDLCDVNLLQLVVTAADFRHPLCSPQTNTPTTTFQVLKDIYYDTMSISEPTDSYTSVNNKSTTQTFTNYSNTLENILYTRASYWNSFHATEYLNPNIIYKNGEKLFKEHEDLITWMTQTNNTGFLTKNNTAFGNNSYRPNADKIKKARKTYWNALIGTNDLATNIGQARAERFEYHLGWYSPIFLSRHRSNMNFARAYQDVTYNPNCDRGVNNRVWVQPLTKPTTEFDEKRCKCVVQHLPLWAALYCYQDFVEEELGSSSEILNSCLLVLQCPYTFPPMYDKKLPDKGFVFYDSLFGDGKMSDGRGQVDIFWQQRWYPRLATQMQVMHDITMTGPFSYRDELVSTQLTAKYTFDFMWGGNMISTQIIKNPCKDSGLEPAYPGRQRRDLQIVDPYSMGPQFSFHNWDYRHGLFGQDAIDRVSKQPKDDADYPNPYKRPRYFPPTDQAAQEQEKDFSFLKTAPSNSEESDQEVLQETQVLRFQPEQHKQLHLQLAERQRIGEQLRYLLQQMFKTQANLHLNPYTFTQL

SEQ ID NO: 52 - protein, 203 aa, Alphatorquevirus sp.
MAWSWWWQRWRRRRWKPRRRRWRRLRWRRPRRAVRRRRRGRRIIKNPCKDSGLEPAYPGRQRRDLQIVDPYSMGPQFSFHNWDYRHGLFGQDAIDRVSKQPKDDADYPNPYKRPRYFPPTDQAAQEQEKDFSFLKTAPSNSEESDQEVLQETQVLRFQPEQHKQLHLQLAERQRIGEQLRYLLQQMFKTQANLHLNPYTFTQL

SEQ ID NO: 53 - protein, 116 aa, Alphatorquevirus sp.
MAWSWWWQRWRRRRWKPRRRRWRRLRWRRPRRAVRRRRRGRRTKPPKSKKKTSVSSKQHRRTQKRAIKKSSKKRKYSDSSQSSTSNSTCSSQSGSESESNSDTYSNRCSKLRPIST

SEQ ID NO: 54 - DNA, 2979 bp, Betatorquevirus sp.
  source 1..2979: mol_type = genomic DNA
taataaatattcaacaggaaaaccacctaatttaaattgccgaccacaaaccgtcacttagttcccctttttgcaacaacttctgcttttttccaactgccggaaaaccacataatttgcatggctaaccacaaactgatatgctaattaacttccacaaaacaacttccccttttaaaaccacacctacaaattaattattaaacacagtcacatcctgggaggtactaccacactataataccaagtgcacttccgaatggctgagtttatgccgctagacggagaacgcatcagttactgactgcggactgaacttgggcgggtgccgaaggtgagtgaaaccaccgaagtcaaggggcaattcgggctagttcagtctagcggaacgggcaagaaacttaaaattattttatttttcagatgagcgactgctttaaaccaacatgctacaacaacaaaacaaagcaaactcactggattaataacctgcatttaacccacgacctgatctgcttctgcccaacaccaactagacacttattactagctttagcagaacaacaagaaacaattgaagtgtctaaacaagaaaaagaaaaaataacaagatgccttattactacagaagaagacggtacaactacagacgtcctagatggtatggacgaggttggattagacgcccttttcgcagaagatttcgaagaaaaagaagggtaagacctacttatactactattcctctaaagcaatggcaaccgccatataaaagaacatgctatataaaaggacaagactgtttaatatactatagcaacttaagactgggaatgaatagtacaatgtatgaaaaaagtattgtacctgtacattggccgggagggggttctttttctgtaagcatgttaactttagatgccttgtatgatatacataaactttgtagaaactggtggacatccacaaaccaagacttaccactagtaagatataaaggatgcaaaataacattttatcaaagcacatttacagactacatagtaagaatacatacagaactaccagctaacagtaacaaactaacatacccaaacacacatccactaatgatgatgatgtctaagtacaaacacattatacctagtagacaaacaagaagaaaaaagaaaccatacacaaaaatatttgtaaaaccacctccgcaatttgaaaacaaatggtactttgctacagacctctacaaaattccattactacaaatacactgcacagcatgcaacttacaaaacccatttgtaaaaccagacaaattatcaaacaatgttacattatggtcactaaacaccataagcatacaaaatagaaacatgtcagtggatcaaggacaatcatggccatttaaaatactaggaacacaaagcttttatttttacttttacaccggagcaaacctaccaggtgacacaacacaaataccagtagcagacctattaccactaacaaacccaagaataaacagaccaggacaatcactaaatgaggcaaaaattacagaccatattactttcacagaatacaaaaacaaatttacaaattattggggtaacccatttaataaacacattcaagaacacctagatatgatactatactcactaaaaagtccagaagcaataaaaaacgaatggacaacagaaaacatgaaatggaaccaattaaacaatgcaggaacaatggcattaacaccatttaacgagccaatattcacacaaatacaatataacccagatagagacacaggagaagacactcaattatacctactctctaacgctacaggaacaggatgggacccaccaggaattccagaattaatactagaaggatttccactatggttaatatattggggatttgcagactttcaaaaaaacctaaaaaaagtaacaaacatagacacaaattacatgttagtagcaaaaacaaaatttacacaaaaacctggcacattctacttagtaatactaaatgacacctttgtagaaggcaatagcccatatgaaaaacaacctttacctgaagacaacattaaatggtacccacaagtacaataccaattagaagcacaaaacaaactactacaaactgggccatttacaccaaacatacaaggacaactatcagacaatatatcaatgttttataaattttactttaaatggggaggaagcccaccaaaagcaattaatgttgaaaatcctgcccaccagattcaatatcccataccccgtaacgagcatgaaacaacttcgttacagagtccaggggaagccccagaatccatcttatactccttcgactatagacacgggaactacacaacaacagctttgtcacgaattagccaagactgggcacttaaagacactgtttctaaaattacagagccagatcgacagcaactgctcaaacaagccctcgaatgcctgcaaatctcggaagaaacgcaggagaaaaaagaaaaagaagtacagcagctcatcagcaacctcagacagcagcagcagctgtacagagagcgaataatatcattattaaaggaccaataacttttaactgtgtaaaaaaggtgaaattgtttgatgataaaccaaaaaaccgtagatttacacctgaggaatttgaaactgagttacaaatagcaaaatggttaaagagacccccaagatcctttgtaaatgatcctcccttttacccatggttaccacctgaacctgttgtaaactttaagcttaattttactgaataaaggccagcattaattcacttaaggagtctgtttatttaagttaaaccttaataaacggtcaccgcctccctaatacgcaggcgcagaaagggggctccgccccctttaacccccagggggctccgccccctgaaacccccaagggggctacgcccccttacaccccc

SEQ ID NO: 55 - protein, 99 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEG

SEQ ID NO: 56 - protein, 203 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGFNIPYPVTSMKQLRYRVQGKPQNPSYTPSTIDTGTTQQQLCHELAKTGHLKTLFLKLQSQIDSNCSNKPSNACKSRKKRRRKKKKKYSSSSATSDSSSSCTESE

SEQ ID NO: 57 - protein, 219 aa, Betatorquevirus sp.
MSDCFKPTCYNNKTKQTHWINNLHLTHDLICFCPTPTRHLLLALAEQQETIEVSKQEKEKITRCLITTEEDGTTTDVLDGMDEVGLDALFAEDFEEKEGARSTATAQTSPRMPANLGRNAGEKRKRSTAAHQQPQTAAAAVQRANNIIIKGPITFNCVKKVKLFDDKPKNRRFTPEEFETELQIAKWLKRPPRSFVNDPPFYPWLPPEPVVNFKLNFTE

SEQ ID NO: 58 - protein, 666 aa, Betatorquevirus sp.
MPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 59 - protein, 148 aa, Betatorquevirus sp.
MPYYYRRRRYNYRRPRWYGRGWIRRPFRRRFRRKRRIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 60 - protein, 82 aa, Betatorquevirus sp.
MPYYYRRRRYNYRRPRWYGRGWIRRPFRRRFRRKRRSQIDSNCSNKPSNACKSRKKRRRKKKKKYSSSSATSDSSSSCTESE

SEQ ID NO: 61 - DNA, 3242 bp, Gammatorquevirus sp.
  source 1..3242: mol_type = genomic DNA
aggtggagactcttaagctatataaccaagtggggtggcgaatggctgagtttaccccgctagacggtgcagggaccggatcgagcgcagcgaggaggtccccggctgcccgtgggcgggagcccgaggtgagtgaaaccaccgaggtctaggggcaattcgggctagggcagtctagcggaacgggcaagaaacttaaaaatatttcttttacagatgcaaaacctatcagccaaagacttctacaaaccatgcagatacaactgtgaaactaaaaaccaaatgtggatgtctggcattgctgactcccatgacagttggtgtgactgtgatactccttttgctcacctcctggctagtatttttcctcctggtcacacagatcgcacacgaaccatccaagaaatacttaccagagattttaggaaaacatgcctttctggtggggccgacgcaacaaattctggtatggccgaaactatagaagaaaaaagagaagatttccaaaaagaagaaaaagaagattttacagaagaacaaaatatagaagacctgctcgccgccgtcgcagacgcagaaggaaggtaagaagaaaaaaaaaaactcttatagtaagacaatggcagccagactctattgtactctgtaaaattaaagggtatgactctataatatggggagctgaaggcacacagtttcaatgttctacacatgaaatgtatgaatatacaagacaaaagtaccctgggggaggaggatttggtgtacaactttacagcttagagtatttgtatgaccaatggaaacttagaaataatatatggactaaaacaaatcaactcaaagatttgtgtagatacttaaaatgtgttatgaccttttacagacaccaacacatagattttgtaattgtatatgaaagacaacccccatttgaaatagataaactaacatacatgaaatatcatccatatatgttattacaaagaaagcataaaataattttacctagtcaaacaactaatcctagaggtaaattaaaaaaaaagaaaactattaaacctcccaaacaaatgctcagcaaatggttttttcaacaacaatttgctaaatatgatctactacttattgctgcagcagcatgtagtttaagataccctagaataggctgctgcaatgaaaatagaatgataaccttatactgtttaaatactaaattttatcaagatacagaatggggaactacaaaacaggccccccactactttaaaccatatgcaacaattaataaatccatgatatttgtctctaactatggaggtaaaaaaacagaatataacataggccaatggatagaaacagatatacctggagaaggtaatctagcaagatactacagatcaataagtaaagaaggaggttacttttcacctaaaatactgcaagcatatcaaacaaaagtaaagtctgtagactacaaacctttaccaattgttttaggtagatataacccagcaatagatgatggaaaaggcaacaaaatttacttacaaactataatgaatggccattggggcctacctcaaaaaacaccagattatataatagaagaggtccctctttggctaggcttctggggatactataactacttaaaacaaacaagaactgaagctatatttccactacacatgtttgtagtgcaaagcaaatacattcaaacacaacaaacagaaacacctaacaatttttgggcatttatagacaacagctttatacagggcaaaaacccatgggactcagttattacttactcagaacaaaagctatggtttcctacagttgcatggcaactaaaaaccataaatgctatttgtgaaagtggaccatatgtacctaaactagacaatcaaacatatagtacctgggaactagcaactcattactcatttcactttaaatggggtggtccacagatatcagaccaaccagttgaagacccaggaaacaaaaacaaatatgatgtgcccgatacaatcaaagaagcattacaaattgttaacccagcaaaaaacattgctgccacgatgttccatgactgggactacagacggggttgcattacatcaacagctattaaaagaatgcaacaaaacctcccaactgattcatctctcgaatctgattcagactcagaaccagcacccaagaaaaaaagactactaccagtcctccacgacccacaaaagaaaacggaaaagatcaaccaatgtctcctctctctctgcgaagaaagtacatgccaggagcaggaaacggaggaaaacatcctcaagctcatccagcagcagcagcagcagcagcagaaactcaagcacaacctcttagtactaatcaaggacttaaaagtgaaacaaagattattacaactacaaacgggggtactagaataacccttaccagatttaaaccaggatttgagcaagaaactgaaaaagagttagcacaagcatttaacagaccccctagactgttcaaagaagataaacccttttacccctggctacccagatttacaccccttgtaaactttcaccttaattttaaaggctaggcctacactgctcacttagtggtgtatgtttattaaagtttgcaccccagaaaaattgtaaaataaaaaaaaaaaaaaaaaataaaaaattgcaaaaattcggcgctcgcgcgcgctgcgcgcgcgagcgccgtcacgcgccggcgctcgcgcgccgcgcgtatgtgctaacacaccacgcacctagattggggtgcgcgcgtagcgcgcgcaccccaatgcgccccgccctcgttccgacccgcttgcgcgggtcggaccacttcgggctcgggggggcgcgcctgcggcgcttatttactaaacagactccgagtcgccattgggccccccctaagctccgcccccctcatgaatattcataaaggaaaccacaaaattagaattgccgaccacaaactgccatatgctaattagttccccttttacacagtaaaaaggggaagtgggggggcagagcccccccacaccccccgcggggggggcagagccccccccgcaccccccctacgtcacaggccacgcccccgccgccatcttgggtgcggcagggcggggactaaaatggcgggacccaatcattttatactttcactttccaattaaaacccgccacgtcacacaaaag

SEQ ID NO: 62 - protein, 101 aa, Gammatorquevirus sp.
MWMSGIADSHDSWCDCDTPFAHLLASIFPPGHTDRTRTIQEILTRDFRKTCLSGGADATNSGMAETIEEKREDFQKEEKEDFTEEQNIEDLLAAVADAEGR

SEQ ID NO: 63 - protein, 237 aa, Gammatorquevirus sp.
MWMSGIADSHDSWCDCDTPFAHLLASIFPPGHTDRTRTIQEILTRDFRKTCLSGGADATNSGMAETIEEKREDFQKEEKEDFTEEQNIEDLLAAVADAEGRYQTNQLKTQETKTNMMCPIQSKKHYKLLTQQKTLLPRCSMTGTTDGVALHQQLLKECNKTSQLIHLSNLIQTQNQHPRKKDYYQSSTTHKRKRKRSTNVSSLSAKKVHARSRKRRKTSSSSSSSSSSSSRNSSTTS

SEQ ID NO: 64 - protein, 239 aa, Gammatorquevirus sp.
MWMSGIADSHDSWCDCDTPFAHLLASIFPPGHTDRTRTIQEILTRDFRKTCLSGGADATNSGMAETIEEKREDFQKEEKEDFTEEQNIEDLLAAVADAEGRTSTQEKKTTTSPPRPTKENGKDQPMSPLSLRRKYMPGAGNGGKHPQAHPAAAAAAAETQAQPLSTNQGLKSETKIITTTNGGTRITLTRFKPGFEQETEKELAQAFNRPPRLFKEDKPFYPWLPRFTPLVNFHLNFKG

SEQ ID NO: 65 - protein, 673 aa, Gammatorquevirus sp.
MPFWWGRRNKFWYGRNYRRKKRRFPKRRKRRFYRRTKYRRPARRRRRRRRKVRRKKKTLIVRQWQPDSIVLCKIKGYDSIIWGAEGTQFQCSTHEMYEYTRQKYPGGGGFGVQLYSLEYLYDQWKLRNNIWTKTNQLKDLCRYLKCVMTFYRHQHIDFVIVYERQPPFEIDKLTYMKYHPYMLLQRKHKIILPSQTTNPRGKLKKKKTIKPPKQMLSKWFFQQQFAKYDLLLIAAAACSLRYPRIGCCNENRMITLYCLNTKFYQDTEWGTTKQAPHYFKPYATINKSMIFVSNYGGKKTEYNIGQWIETDIPGEGNLARYYRSISKEGGYFSPKILQAYQTKVKSVDYKPLPIVLGRYNPAIDDGKGNKIYLQTIMNGHWGLPQKTPDYIIEEVPLWLGFWGYYNYLKQTRTEAIFPLHMFVVQSKYIQTQQTETPNNFWAFIDNSFIQGKNPWDSVITYSEQKLWFPTVAWQLKTINAICESGPYVPKLDNQTYSTWELATHYSFHFKWGGPQISDQPVEDPGNKNKYDVPDTIKEALQIVNPAKNIAATMFHDWDYRRGCITSTAIKRMQQNLPTDSSLESDSDSEPAPKKKRLLPVLHDPQKKTEKINQCLLSLCEESTCQEQETEENILKLIQQQQQQQQKLKHNLLVLIKDLKVKQRLLQLQTGVLE

SEQ ID NO: 66 - protein, 209 aa, Gammatorquevirus sp.
MPFWWGRRNKFWYGRNYRRKKRRFPKRRKRRFYRRTKYRRPARRRRRRRRKISDQPVEDPGNKNKYDVPDTIKEALQIVNPAKNIAATMFHDWDYRRGCITSTAIKRMQQNLPTDSSLESDSDSEPAPKKKRLLPVLHDPQKKTEKINQCLLSLCEESTCQEQETEENILKLIQQQQQQQQKLKHNLLVLIKDLKVKQRLLQLQTGVLE

SEQ ID NO: 67 - protein, 209 aa, Gammatorquevirus sp.; identical to SEQ ID NO: 66

SEQ ID NO: 68 - protein, 25 aa, synthetic construct
  VARIANT 9: note = Any amino acid
  VARIANT 10: note = Any amino acid
  VARIANT 15: note = Any amino acid
  VARIANT 17: note = Any amino acid
  VARIANT 20: note = Any amino acid
  VARIANT 24: note = Any amino acid
LIJRQWQPXXIRRCXIXGYXPLIXC

SEQ ID NO: 69 - protein, 7 aa, synthetic construct
  VARIANT 3: note = Any amino acid
  VARIANT 4: note = Any amino acid
  VARIANT 6: note = Any amino acid
NYXXHXD

SEQ ID NO: 70 - protein, 9 aa, synthetic construct
  VARIANT 4: note = Any amino acid
  VARIANT 5: note = Any amino acid
FSLXXLYDZ

SEQ ID NO: 71 - protein, 18 aa, synthetic construct
  VARIANT 2: note = Any amino acid
  VARIANT 5: note = Any amino acid
  VARIANT 8: note = Any amino acid
  VARIANT 16: note = Any amino acid
NXWTXSNXDLDLCRYXGC

SEQ ID NO: 72 - protein, 16 aa, synthetic construct
  VARIANT 2: note = Any amino acid
  VARIANT 5: note = Any amino acid
  VARIANT 9: note = Any amino acid
  VARIANT 11: note = Any amino acid
  VARIANT 13: note = Any amino acid
TXPSXHPGXMXLXKHK

SEQ ID NO: 73 - protein, 10 aa, synthetic construct
  VARIANT 5: note = Any amino acid
  VARIANT 9: note = Any amino acid
IPSLXTRPXG

SEQ ID NO: 74 - protein, 18 aa, synthetic construct
  VARIANT 3: note = Any amino acid
  VARIANT 6: note = Any amino acid
  VARIANT 9: note = Any amino acid
  VARIANT 16: note = Any amino acid
RIXPPXLFXDKWYFQXDL

SEQ ID NO: 75 - protein, 8 aa, synthetic construct
  VARIANT 3: note = Any amino acid
  VARIANT 5: note = Any amino acid
LLXIXATA

SEQ ID NO: 76 - protein, 11 aa, synthetic construct
  VARIANT 2: note = Any amino acid
  VARIANT 3: note = Any amino acid
  VARIANT 6: note = Any amino acid
  VARIANT 9: note = Any amino acid
LXXPFXSPXTD

SEQ ID NO: 77 - protein, 14 aa, synthetic construct
  VARIANT 4: note = Any amino acid
  VARIANT 5: note = Any amino acid
  VARIANT 9: note = Any amino acid
  VARIANT 12: note = Any amino acid
YNPXXDKGXGNXIW

SEQ ID NO: 78 - protein, 8 aa, synthetic construct
  VARIANT 7: note = Any amino acid
CPYTZPXL

SEQ ID NO: 79 - protein, 8 aa, synthetic construct
  VARIANT 1: note = Any amino acid
  VARIANT 4: note = Any amino acid
  VARIANT 6: note = Any amino acid
XFGXGXMP

SEQ ID NO: 80 - protein, 8 aa, synthetic construct
  VARIANT 3: note = Any amino acid
  VARIANT 6: note = Any amino acid
  VARIANT 8: note = Any amino acid
HQXEVXEX

SEQ ID NO: 81 - protein, 12 aa, synthetic construct
  VARIANT 3: note = Any amino acid
  VARIANT 5: note = Any amino acid
  VARIANT 7: note = Any amino acid
KYXFXFXWGGNP

SEQ ID NO: 82 - protein, 8 aa, synthetic construct
  VARIANT 5: note = Any amino acid
HSWDXRRG

SEQ ID NO: 83 - protein, 7 aa, synthetic construct
  VARIANT 5: note = Any amino acid
AIKRXQQ

SEQ ID NO: 84 - protein, 8 aa, synthetic construct
  VARIANT 1: note = Any amino acid
  VARIANT 5: note = Any amino acid
  VARIANT 6: note = Any amino acid
XQZQXXLR

SEQ ID NO: 85 - protein, 9 aa, synthetic construct
  VARIANT 3: note = Any amino acid
  VARIANT 6: note = Any amino acid
  VARIANT 7: note = Any amino acid
PRXJQXXDP

SEQ ID NO: 86 - protein, 8 aa, synthetic construct
  VARIANT 5: note = Any amino acid
HSWDXRRG

SEQ ID NO: 87 - protein, 7 aa, synthetic construct
  VARIANT 5: note = Any amino acid
AIKRXQQ

SEQ ID NO: 88 - protein, 7 aa, synthetic construct
  VARIANT 4: note = Any amino acid
  VARIANT 5: note = Any amino acid
QZQXXLR

SEQ ID NO: 89 - protein, 7 aa, synthetic construct
  VARIANT 2: note = Any amino acid
KXKRRRR

SEQ ID NO: 90 - protein, 13 aa, synthetic construct
  VARIANT 3: note = Any amino acid
  VARIANT 6: note = Any amino acid
  VARIANT 7: note = Any amino acid
  VARIANT 10: note = Any amino acid
  VARIANT 11: note = Any amino acid
PIXSLXXYKXXTR

SEQ ID NO: 91 - protein, 12 aa, synthetic construct
  VARIANT 3: note = Any amino acid
  VARIANT 10: note = Any amino acid
LAXQLLKECXKN

SEQ ID NO: 92 - protein, 6 aa, synthetic construct
  VARIANT 4: note = Any amino acid
HLNXLA

SEQ ID NO: 93 - protein, 5 aa, synthetic construct
DRPPR

SEQ ID NO: 94 - protein, 9 aa, synthetic construct
  VARIANT 2: note = Any amino acid
  VARIANT 8: note = Any amino acid
DXPFYPWXP

SEQ ID NO: 95 - protein, 7 aa, synthetic construct
  VARIANT 2: note = Any amino acid
  VARIANT 6: note = Any amino acid
VXFKLXF

SEQ ID NO: 96 - protein, 15 aa, synthetic construct
  VARIANT 2: note = Any amino acid
  VARIANT 9: note = Any amino acid
  VARIANT 14: note = Any amino acid
WXPPVHBVXGIERXW

SEQ ID NO: 97 - protein, 6 aa, synthetic construct
  VARIANT 6: note = Any amino acid
AKRKLX

SEQ ID NO: 98 - protein, 10 aa, synthetic construct
  VARIANT 4: note = Any amino acid
  VARIANT 7: note = Any amino acid
  VARIANT 8: note = Any amino acid
PSSXDWXXEY

SEQ ID NO: 99 - protein, 5 aa, synthetic construct
DRPPR

SEQ ID NO: 100 - protein, 5 aa, synthetic construct
PFYPW

SEQ ID NO: 101 - protein, 8 aa, synthetic construct
  VARIANT 3: note = Any amino acid
  VARIANT 7: note = Any amino acid
NVXFKLXF

SEQ ID NO: 102 - protein, 25 aa, synthetic construct
  VARIANT 2: note = Any amino acid
  VARIANT 3: note = Any amino acid
  VARIANT 4: note = Any amino acid
  VARIANT 5: note = Any amino acid
  VARIANT 9: note = Any amino acid
  VARIANT 10: note = Any amino acid
  VARIANT 11: note = Any amino acid
  VARIANT 12: note = Any amino acid
  VARIANT 13: note = Any amino acid
  VARIANT 15: note = Any amino acid
  VARIANT 17: note = Any amino acid
  VARIANT 19: note = Any amino acid
  VARIANT 20: note = Any amino acid
  VARIANT 21: note = Any amino acid
JXXXXWQPXXXXXCXIXGXXXJWQP

SEQ ID NO: 103 - protein, 15 aa, synthetic construct
  VARIANT 2: note = Any amino acid
  VARIANT 4: note = Any amino acid
  VARIANT 5: note = Any amino acid
  VARIANT 6: note = Any amino acid
  VARIANT 8: note = Any amino acid
  VARIANT 9: note = Any amino acid
  VARIANT 10: note = Any amino acid
  VARIANT 11: note = Any amino acid
  VARIANT 13: note = Any amino acid
NXWXXXNXXXXLXRY

SEQ ID NO: 104 - protein, 8 aa, synthetic construct
  VARIANT 4: note = Any amino acid
  VARIANT 5: note = Any amino acid
  VARIANT 7: note = Any amino acid
YNPXXDXG

SEQ ID NO: 105 - DNA, 71 bp, synthetic construct
  source 1..71: mol_type = other DNA
cgggtgccgkaggtgagtttacacaccgmagtcaaggggcaattcgggctcrggactggccgggcyhtggg

SEQ ID NO: 106 - DNA, 71 bp, synthetic construct
  source 1..71: mol_type = other DNA
cgggtgccggaggtgagtttacacaccgcagtcaaggggcaattcgggctcgggactggccgggctwtggg

SEQ ID NO: 107 - DNA, 71 bp, Torque teno virus
  source 1..71: mol_type = genomic DNA
cgggtgccgtaggtgagtttacacaccgcagtcaaggggcaattcgggctcgggactggccgggctatggg

SEQ ID NO: 108 - DNA, 71 bp, Torque teno virus
  source 1..71: mol_type = genomic DNA
cgggtgccggaggtgagtttacacaccgcagtcaaggggcaattcgggctcgggactggccgggccctggg

SEQ ID NO: 109 - DNA, 71 bp, Torque teno virus
  source 1..71: mol_type = genomic DNA
cgggtgccggaggtgagtttacacaccgcagtcaaggggcaattcgggctcgggactggccgggctttggg

SEQ ID NO: 110 - DNA, 71 bp, Torque teno virus
  source 1..71: mol_type = genomic DNA
cgggtgccggaggtgagtttacacaccgcagtcaaggggcaattcgggctcgggactggccgggctatggg

SEQ ID NO: 111 - DNA, 71 bp, Torque teno virus
  source 1..71: mol_type = genomic DNA
cgggtgccggaggtgagtttacacaccgaagtcaaggggcaattcgggctcaggactggccgggctttggg

SEQ ID NO: 112 - DNA, 71 bp, synthetic construct
  source 1..71: mol_type = other DNA
cgggtgccggaggtgagtttacacaccgcagtcaaggggcaattcgggctcgggactggccgggcyhtggg

SEQ ID NO: 113 - DNA, 71 bp, Torque teno virus; identical to SEQ ID NO: 107
  source 1..71: mol_type = genomic DNA

SEQ ID NO: 114 - DNA, 70 bp, Torque teno virus
  source 1..70: mol_type = genomic DNA
cgggtgccggaggtgagtttacacaccgcagtcaaggggcaattcgggctcgggactggccgggcccggg

SEQ ID NO: 115 - DNA, 71 bp, Torque teno virus; identical to SEQ ID NO: 111
  source 1..71: mol_type = genomic DNA

SEQ ID NO: 116 - DNA, 69 bp, Torque teno virus
  source 1..69: mol_type = genomic DNA
cgggtgccggaggtgagtttacacaccgcagtcaaggggcaattcgggctcgggaggccgggccatggg

SEQ ID NO: 117 - DNA, 71 bp, Torque teno virus
  source 1..71: mol_type = genomic DNA
cgggtgccggaggtgagtttacacaccgcagtcaaggggcaattcgggctcgggactggccgggccccggg

SEQ ID NO: 118 - DNA, 71 bp, Torque teno virus; identical to SEQ ID NO: 110
  source 1..71: mol_type = genomic DNA

SEQ ID NO: 119 - DNA, 71 bp, Torque teno virus
  source 1..71: mol_type = genomic DNA
cgggtgccgaaggtgagtttacacaccgcagtcaaggggcaattcgggctcgggactggccgggctatggg

SEQ ID NO: 120 - DNA, 117 bp, synthetic construct
  source 1..117: mol_type = other DNA
  misc_difference 10: note = This base may be absent
  misc_difference 12: note = This base may be absent
  misc_difference 30: note = This base may be absent
  misc_difference 31: note = This base may be absent
  misc_difference 32: note = This base may be absent
  misc_difference 34: note = This base may be absent
  misc_difference 43: note = This base may be absent
  misc_difference 44: note = This base may be absent
  misc_difference 45: note = This base may be absent
  misc_difference 46: note = This base may be absent
  misc_difference 52: note = This base may be absent
  misc_difference 53: note = This base may be absent
  misc_difference 54: note = This base may be absent
  misc_difference 70: note = This base may be absent
  misc_difference 71: note = This base may be absent
  misc_difference 89: note = This base may be absent
  misc_difference 90: note = This base may be absent
  misc_difference 103: note = This base may be absent
cggcggsggsgcsscgcgctdcgcgcgcsgcccrsyrggggrdssmmwgcskcscccccccscgcgcatgcgcrcgggkcccccccccyvsggggggctccgcccccccggcccccc

SEQ ID NO: 121 - DNA, 169 bp, synthetic construct
  source 1..169: mol_type = other DNA
  misc_difference 20: note = a, c, t, g, unknown or other
  misc_difference 22: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 62: note = a, c, t, g, unknown or other
  misc_difference 64: note = a, c, t, g, unknown or other
  misc_difference 97: note = a, c, t, g, unknown or other
  misc_difference 98: note = a, c, t, g, unknown or other
gccgccgcggcggcggsggngnsgcgcgctdcgcgcgcsnnncrccrgggggnnnncwgcsncncccccccccgcgcatgcgcgggkcccccccccnncggggggctccgccccccggcccccccccgtgctaaacccaccgcgcatgcgcgaccacgcccccgccgcc

SEQ ID NO: 122 - DNA, 79 bp, synthetic construct
  source 1..79: mol_type = other DNA
  misc_difference 20: note = a, c, t, g, unknown or other
  misc_difference 22: note = a, c, t, g, unknown or other
  misc_difference 40: note = a, c, t, g, unknown or other
  misc_difference 41: note = a, c, t, g, unknown or other
  misc_difference 42: note = a, c, t, g, unknown or other
  misc_difference 53: note = a, c, t, g, unknown or other
  misc_difference 54: note = a, c, t, g, unknown or other
  misc_difference 55: note = a, c, t, g, unknown or other
  misc_difference 56: note = a, c, t, g, unknown or other
  misc_difference 62: note = a, c, t, g, unknown or other
  misc_difference 64: note = a, c, t, g, unknown or other
gccgccgcggcggcggsggngnsgcgcgctdcgcgcgcsnnncrccrgggggnnnncwgcsncncccccccccgcgcat

SEQ ID NO: 123 - DNA, 31 bp, synthetic construct
  source 1..31: mol_type = other DNA
  misc_difference 18: note = a, c, t, g, unknown or other
  misc_difference 19: note = a, c, t, g, unknown or other
gcgcgggkcccccccccnncggggggctccg

SEQ ID NO: 124 - DNA, 59 bp, synthetic construct
  source 1..59: mol_type = other DNA
ccccccggcccccccccgtgctaaacccaccgcgcatgcgcgaccacgcccccgccgcc

SEQ ID NO: 125 - DNA, 156 bp, Torque teno virus
  source 1..156: mol_type = genomic DNA
gcggcgggggggcggccgcgttcgcgcgccgcccaccagggggtgctgcgcgcccccccccgcgcatgcgcggggcccccccccgggggggctccgcccccccggcccccccccgtgctaaacccaccgcgcatgcgcgaccacgcccccgccgcc

SEQ ID NO: 126-128 - skipped (3 entries)

SEQ ID NO: 129 - DNA, 25 bp, Torque teno virus
  source 1..25: mol_type = genomic DNA
ttcgcgcgccgcccaccagggggtg

SEQ ID NO: 130 - skipped

SEQ ID NO: 131 - DNA, 17 bp, Torque teno virus
  source 1..17: mol_type = genomic DNA
cgcccccccccgcgcat

SEQ ID NO: 132 - DNA, 17 bp, Torque teno virus
  source 1..17: mol_type = genomic DNA
gcgcggggccccccccc

SEQ ID NO: 133 - DNA, 72 bp, Torque teno virus
  source 1..72: mol_type = genomic DNA
gggggggctccgcccccccggcccccccccgtgctaaacccaccgcgcatgcgcgaccacgcccccgccgcc

SEQ ID NO: 134 - DNA, 115 bp, Torque teno virus
  source 1..115: mol_type = genomic DNA
cggcggcggcggcgcgcgcgctgcgcgcgcgcgccgggggggcgccagcgcccccccccccgcgcatgcacgggtccccccccccacggggggctccgccccccggccccccccc

SEQ ID NO: 135 - DNA, 14 bp, Torque teno virus
  source 1..14: mol_type = genomic DNA
cggcggcggcggcg

SEQ ID NO: 136 - DNA, 17 bp, Torque teno virus
  source 1..17: mol_type = genomic DNA
cgcgcgctgcgcgcgcg

SEQ ID NO: 137 - DNA, 19 bp, Torque teno virus
  source 1..19: mol_type = genomic DNA
cgccgggggggcgccagcg

SEQ ID NO: 138 - DNA, 17 bp, Torque teno virus
  source 1..17: mol_type = genomic DNA
cccccccccccgcgcat

SEQ ID NO: 139 - DNA, 31 bp, Torque teno virus
  source 1..31: mol_type = genomic DNA
gcacgggtccccccccccacggggggctccg

SEQ ID NO: 140 - DNA, 17 bp, Torque teno virus
  source 1..17: mol_type = genomic DNA
ccccccggccccccccc

SEQ ID NO: 141 - DNA, 121 bp, Torque teno virus
  source 1..121: mol_type = genomic DNA
ccgtcggcgggggggccgcgcgctgcgcgcgcggcccccgggggaggcacagcctcccccccccgcgcgcatgcgcgcgggtccccccccctccggggggctccgccccccggcccccccc

SEQ ID NO: 142 - DNA, 37 bp, Torque teno virus
  source 1..37: mol_type = genomic DNA
ccgtcggcgggggggccgcgcgctgcgcgcgcggccc

SEQ ID NO: 143 - DNA, 84 bp, Torque teno virus
  source 1..84: mol_type = genomic DNA
ccgggggaggcacagcctcccccccccgcgcgcatgcgcgcgggtccccccccctccggggggctccgccccccggcccccccc

SEQ ID NO: 144 - DNA, 104 bp, Torque teno virus
  source 1..104: mol_type = genomic DNA
cggcggcggcgcgcgcgctacgcgcgcgcgccggggggctgccgccccccccccgcgcatgcgcggggcccccccccgcggggggctccgccccccggcccccc

SEQ ID NO: 145 - DNA, 11 bp, Torque teno virus
  source 1..11: mol_type = genomic DNA
cggcggcggcg

SEQ ID NO: 146 - DNA, 17 bp, Torque teno virus
  source 1..17: mol_type = genomic DNA
cgcgcgctacgcgcgcg

SEQ ID NO: 147 - DNA, 10 bp, Torque teno virus
  source 1..10: mol_type = genomic DNA
cgccgggggg

SEQ ID NO: 148 - skipped

SEQ ID NO: 149 - DNA, 15 bp, Torque teno virus
  source 1..15: mol_type = genomic DNA
cccccccccgcgcat

SEQ ID NO: 150 - DNA, 17 bp, Torque teno virus
  source 1..17: mol_type = genomic DNA
gcgcggggccccccccc

SEQ ID NO: 151 - DNA, 13 bp, Torque teno virus
  source 1..13: mol_type = genomic DNA
gcggggggctccg

SEQ ID NO: 152 - DNA, 14 bp, Torque teno virus
  source 1..14: mol_type = genomic DNA
ccccccggcccccc

SEQ ID NO: 153 - DNA, 122 bp, Torque teno virus
  source 1..122: mol_type = genomic DNA
gccgccgcggcggcggggggcggcgcgctgcgcgcgccgcccagtagggggagccatgcgcccccccccgcgcatgcgcggggcccccccccgcggggggctccgccccccggccccccccg

SEQ ID NO: 154 - DNA, 19 bp, Torque teno virus
  source 1..19: mol_type = genomic DNA
gccgccgcggcggcggggg

SEQ ID NO: 155 - DNA, 41 bp, Torque teno virus
  source 1..41: mol_type = genomic DNA
gcggcgcgctgcgcgcgccgcccagtagggggagccatgcg

SEQ ID NO: 156 - DNA, 15 bp, Torque teno virus
  source 1..15: mol_type = genomic DNA
cccccccccgcgcat

SEQ ID NO: 157 - DNA, 17 bp, Torque teno virus
  source 1..17: mol_type = genomic DNA
gcgcggggccccccccc

SEQ ID NO: 158 - DNA, 13 bp, Torque teno virus
  source 1..13: mol_type = genomic DNA
gcggggggctccg

SEQ ID NO: 159 - DNA, 17 bp, Torque teno virus
  source 1..17: mol_type = genomic DNA
ccccccggccccccccg

SEQ ID NO: 160 - DNA, 36 bp, Torque teno virus
  source 1..36: mol_type = genomic DNA
cgcgctgcgcgcgccgcccagtagggggagccatgc

SEQ ID NO: 161 - DNA, 78 bp, Torque teno virus
  source 1..78: mol_type = genomic DNA
ccgccatcttaagtagttgaggcggacggtggcgtgagttcaaaggtcaccatcagccacacctactcaaaatggtgg

SEQ ID NO: 162 - DNA, 172 bp, Torque teno virus
  source 1..172: mol_type = genomic DNA
cttaagtagttgaggcggacggtggcgtgagttcaaaggtcaccatcagccacacctactcaaaatggtggacaatttcttccgggtcaaaggttacagccgccatgttaaaacacgtgacgtatgacgtcacggccgccattttgtgacacaagatggccgacttccttcc

SEQ ID NO: 163 - DNA, 36 bp, synthetic construct; identical to SEQ ID NO: 160
  source 1..36: mol_type = other DNA

SEQ ID NO: 164 - DNA, 36 bp, synthetic construct
  source 1..36: mol_type = other DNA
gcgctdcgcgcgcgcgccggggggctgcgccccccc

SEQ ID NO: 165 - DNA, 36 bp, Torque teno virus
  source 1..36: mol_type = genomic DNA
gcgcttcgcgcgccgcccactagggggcgttgcgcg

SEQ ID NO: 166 - DNA, 36 bp, Torque teno virus
  source 1..36: mol_type = genomic DNA
gcgctgcgcgcgccgcccagtagggggcgcaatgcg

SEQ ID NO: 167 - DNA, 36 bp, Torque teno virus
  source 1..36: mol_type = genomic DNA
gcgctgcgcgcgcggcccccgggggaggcattgcct

SEQ ID NO: 168 - DNA, 36 bp, Torque teno virus
  source 1..36: mol_type = genomic DNA
gcgctgcgcgcgcgcgccgggggggcgccagcgccc

SEQ ID NO: 169 - DNA, 36 bp, Torque teno virus
  source 1..36: mol_type = genomic DNA
gcgcttcgcgcgcgcgccggggggctccgccccccc

SEQ ID NO: 170 - DNA, 36 bp, Torque teno virus
  source 1..36: mol_type = genomic DNA
gcgcttcgcgcgcgcgccggggggctgcgccccccc

SEQ ID NO: 171 - DNA, 36 bp, Torque teno virus
  source 1..36: mol_type = genomic DNA
gcgctacgcgcgcgcgccggggggctgcgccccccc

SEQ ID NO: 172 - DNA, 36 bp, Torque teno virus
  source 1..36: mol_type = genomic DNA
gcgctacgcgcgcgcgccggggggctctgccccccc

SEQ ID NO: 173 - protein, 680 aa, Alphatorquevirus sp.; identical to SEQ ID NO: 6

SEQ ID NO: 174 - protein, 66 aa, Alphatorquevirus sp.
TAWWWGRWRRRWRRRRPWRPRLRRRRARRAFPRRRRRRFVSRRWRRPYRRRRRRGRRRRRRRRRHK

SEQ ID NO: 175 - protein, 211 aa, Alphatorquevirus sp.
PTLVLRQWQPDVIRHCKITGRMPLIICGKGSTQFNYITHADDITPRGASYGGNFTNMTFSLEAIYEQFLYHRNRWSASNHDLELCRYKGTTLKLYRHPDVDYIVTYSRTGPFEISHMTYLSTHPLLMLLNKHHIVVPSLKTKPRGRKAIKVRIRPPKLMNNKWYFTRDFCNIGLFQLWATGLELRNPWLRMSTLSPCIGFNVLKNSIYTNL

SEQ ID NO: 176 - protein, 70 aa, Alphatorquevirus sp.
SNLPQHREDRLNIINNTLHPHDITGPNNKKWQYTYTKLMAPIYYSANRASTYDLLREYGLYSPYYLNPTR

SEQ ID NO: 177 - protein, 166 aa, Alphatorquevirus sp.
INLDWMTPYTHVRYNPLVDKGFGNRIYIQWCSEADVSYNRTKSKCLLQDMPLFFMCYGYIDWAIKNTGVSSLARDARICIRCPYTEPQLVGSTEDIGFVPITETFMRGDMPVLAPYIPLSWFCKWYPNIAHQKEVLEAIISCSPFMPRDQGMNGWDITIGYKMDFL

SEQ ID NO: 178 - protein, 167 aa, Alphatorquevirus sp.
WGGSPLPSQPIDDPCQQGTHPIPDPDKHPRLLQVSNPKLLGPRTVFHKWDIRRGQFSKRSIKRVSEYSSDDESLAPGLPSKRNKLDSAFRGENPEQKECYSLLKALEEEETPEEEEPAPQEKAQKEELLHQLQLQRRHQRVLRRGLKLVFTDILRLRQGVHWNPELT

SEQ ID NO: 179 - protein, 747 aa, Alphatorquevirus sp.; identical to SEQ ID NO: 13

SEQ ID NO: 180 - protein, 69 aa, Alphatorquevirus sp.
MAYWWGRRRRWRRWRRRRRPLRRRRRWRRRRRWPRRRRWRRRRRRARPARRYRRRRGRRRVRRRRRPQK

SEQ ID NO: 181 - protein, 210 aa, Alphatorquevirus sp.
LVLTQWNPQTVRKCVIRGFLPLFFCGQGAYHRNFTDHYDDVFPKGPSGGGHGSMVFNLSFLYQEFKKHHNKWSRSNLDFDLVRYKGTVIKLYRHQDFDYIVWISRTPPFQESLLTVMTHQPSVMLQAKKCIIVKSYRTHPGGKPYVTAKVRPPRLLTDKWYFQSDFCNVPLFSLQFALAELRFPICSPQTDTNCINFLVLDDIYYKFLDN

SEQ ID NO: 182 - protein, 132 aa, Alphatorquevirus sp.
KPKQSSDPNDENRIKFWHGLWSTMRYLNTTYINTLFPGTDSLVAAKDTDNSVNKYPSTATKQPYKDSQYMQNIWNTSKIHALYTWVAETNYKRLQAYYTQTYGGYQRQFFTGKQYWDYRVGMFSPAFLSPSR

SEQ ID NO: 183 - protein, 167 aa, Alphatorquevirus sp.
LNPQNPGAYTEVSYNPWTDEGTGNVVCLQYLTKETSDYKPGGGSKFCIEGVPLWAALVGYVDMCKKEGKDPGIRLNCLLLVKCPYTKPQLYDKKNPEKLFVPYSYNFGHGKMPGGDKYIPIEFKDRWYPCLLHQEEWIEDIVRSGPFVPKDMPSSVTCMMRYSSLFN

SEQ ID NO: 184 - protein, 169 aa, Alphatorquevirus sp.
WGGNIIQEQAVEDPCKKGTFVVPGTSGIARILQVSNPAKQTPTTTWHSWDWRRSLFTETGLKRMREQQPYDELSYTGPKKPKLSLPAGPAVPGAAVASSWWETKQVTSPDVSETETEAEAHQEEETEPEEGVQLQQLWEQQLLQKRQLGVVFQQLLRLRQGAEIHPGLV

SEQ ID NO: 185 - protein, 743 aa, Alphatorquevirus sp.; identical to SEQ ID NO: 21

SEQ ID NO: 186 - protein, 68 aa, Alphatorquevirus sp.
MAWGWWKRRRRWWFRKRWTRGRLRRRWPRSARRRPRRRRVRRRRRWRRGRRKTRTYRRRRRFRRRGRK

SEQ ID NO: 187 - protein, 212 aa, Alphatorquevirus sp.
AKLIIKLWQPAVIKRCRIKGYIPLIISGNGTFATNFTSHINDRIMKGPFGGGHSTMRFSLYILFEEHLRHMNFWTRSNDNLELTRYLGASVKIYRHPDQDFIVIYNRRTPLGGNIYTAPSLHPGNAILAKHKILVPSLQTRPKGRKAIRLRIAPPTLFTDKWYFQKDIADLTLFNIMAVEADLRFPFCSPQTDNTCISFQVLSSVYNNYLSI

SEQ ID NO: 188 - protein, 133 aa, Alphatorquevirus sp.
NTFNNDNSDSKLKEFLNKAFPTTGTKGTSLNALNTFRTEGCISHPQLKKPNPQINKPLESQYFAPLDALWGDPIYYNDLNENKSLNDIIEKILIKNMITYHAKLREFPNSYQGNKAFCHLTGIYSPPYLNQGR

SEQ ID NO: 189 - protein, 166 aa, Alphatorquevirus sp.
ISPEIFGLYTEIIYNPYTDKGTGNKVWMDPLTKENNIYKEGQSKCLLTDMPLWTLLFGYTDWCKKDTNNWDLPLNYRLVLICPYTFPKLYNEKVKDYGYIPYSYKFGAGQMPDGSNYIPFQFRAKWYPTVLHQQQVMEDISRSGPFAPKVEKPSTQLVMKYCFNFN

SEQ ID NO: 190 - protein, 164 aa, Alphatorquevirus sp.
WGGNPIIEQIVKDPSFQPTYEIPGTGNIPRRIQVIDPRVLGPHYSFRSWDMRRHTFSRASIKRVSEQQETSDLVFSGPKKPRVDIPKQETQEESSHSLQRESRPWETEEESETEALSQESQEVPFQQQLQQQYQEQLKLRQGIKVLFEQLIRTQQGVHVNPCLR

SEQ ID NO: 191 - protein, 780 aa, Alphatorquevirus sp.; identical to SEQ ID NO: 28

SEQ ID NO: 192 - protein, 74 aa, Alphatorquevirus sp.
MAWWGWRRRWWRPKRRWRWRRARRRRRVPARRPRRAFRRYRTRTVRRRRRGRRRGYRRRYRLRRYARRRFRRKK

SEQ ID NO: 193 - protein, 210 aa, Alphatorquevirus sp.
IVLTQWNPQTTRKCIIRGMMPVLWAGMGTGGRNYAVRSDDYVVNKGFGGSFATETFSLKVLYDQFQRGFNRWSHTNEDLDLARYRGCRWTFYRHKDTDFIVYFTNNPPMKTNQFSAPLTTPGMLMRSKYKVLIPSFQTRPKGRKTVTVKIRPPKLFQDKWYTQQDLCSVPLVQLNVTAADFTHPFGSPLTETPCVEFQVLGDLYNTCLNI

SEQ ID NO: 194 - protein, 161 aa, Alphatorquevirus sp.
DLPQFSELGEITSAYSKPNSNNLKELYKELFTKATSGHYWQTFITNSMVRAHIDADKAKEAQRASTTPSYNNDPFPTIPVKSEFAQWKKKFTDTRDSPFLFATYHPEAIKDTIMKMRENNFKLETGPNDKYGDYTAQYQGNTHMLDYYLGFYSPIFLSDGR

SEQ ID NO: 195 - protein, 166 aa, Alphatorquevirus sp.
SNVEFFTAYRDIVYNPFLDKAQGNMVWFQYHTKTDNKFKKPECHWEIKDMPLWALLNGYVDYLETQIQYGDLSKEGKVLIRCPYTKPALVDPRDDTAGYVVYNRNFGRGKWIDGGGYIPLHERTKWYVMLRYQTDVFHDIVTCGPWQYRDDNKNSQLVAKYRFSFI

SEQ ID NO: 196 - protein, 169 aa, Alphatorquevirus sp.
WGGNTVHSQVIRNPCKDNQVSGPRRQPRDIQVVDPQRITPPWVLHSFDQRRGLFTETALRRLLQEPLPGEYAVSTLRTPLLFLPSEYQREDGAAESASGSPAKRPRIWSEESQTETISSEENPAETTRELLQRKLREQRALQFQLQHFAVQLAKTQANLHVNPLLSFPQ

SEQ ID NO: 197 - protein, 761 aa, Alphatorquevirus sp.; identical to SEQ ID NO: 36

SEQ ID NO: 198 - protein, 75 aa, Alphatorquevirus sp.
MAYWFRRWGWRPRRRWRRWRRRRRRLPRRRTRRAVRGLGRRRKPRVRRRRRTRRRTYRRGWRRRRYIRRGRRKKK

SEQ ID NO: 199 - protein, 209 aa, Alphatorquevirus sp.
LILTQWNPAIVKRCNIKGGLPIIICGEPRAAFNYGYHMEDYTPQPFPFGGGMSTVTFSLKALYDQYLKHQNRWTFSNDQLDLARYRGCKLRFYRSPVCDFIVHYNLIPPLKMNQFTSPNTHPGLLMLSKHKIIIPSFQTRPGGRRFVKIRLNPPKLFEDKWYTQQDLCKVPLVSITATAADLRYPFCSPQTNNPCTTFQVLRKNYNTVI

SEQ ID NO: 200 - protein, 148 aa, Alphatorquevirus sp.
GTSVKDQESTQDFENWLYKTDSHYQTFATEAQLGRIPAFNPDGTKNTKQQSWQDNWSKKNSPWTGNSGTYPQTTSEMYKIPYDSNFGFPTYRAQKDYILERRQCNFNYEVNNPVSKKVWPQPSTTTPTVDYYEYHCGWFSNIFIGPNR

SEQ ID NO: 201 - protein, 167 aa, Alphatorquevirus sp.
YNLQFQTAYVDTTYNPLMDKGKGNKIWFQYLSKKGTDYNEKQCYCTLEDMPLWAICFGYTDYVETQLGPNVDHETAGLIIMICPYTQPPMYDKNRPNWGYVVYDTNFGNGKMPSGSGQVPVYWQCRWRPMLWFQQQVLNDISKTGPYAYRDEYKNVQLTLYYNFIFN

SEQ ID NO: 202 - protein, 162 aa, Alphatorquevirus sp.
WGGDMYYPQVVKNPCGDSGIVPGSGRFTREVQVVSPLSMGPAYIFHYFDSRRGFFSEKALKRMQQQQEFDESFTFKPKRPKLSTAAAEILQLEEDSTSGEGKSPLQQEEKEVEVLQTPTVQLQLQRNIQEQLAIKQQLQFLLLQLLKTQSNLHLNPQFLSPS

SEQ ID NO: 203 - protein, 746 aa, Alphatorquevirus sp.; identical to SEQ ID NO: 44

SEQ ID NO: 204 - protein, 77 aa, Alphatorquevirus sp.
MAWGWWRWRRRWPARRWRRRRRRRPVRRTRARRPARRYRRRRTVRTRRRRWGRRRYRRGWRRRTYVRKGRHRKKKKR

SEQ ID NO: 205 - protein, 209 aa, Alphatorquevirus sp.
LILRQWQPATRRRCTITGYLPIVFCGHTRGNKNYALHSDDYTPQGQPFGGALSTTSFSLKVLFDQHQRGLNKWSFPNDQLDLARYRGCKFIFYRTKQTDWVGQYDISEPYKLDKYSCPNYHPGNMIKAKHKFLIPSYDTNPRGRQKIIVKIPPPDLFVDKWYTQEDLCSVNLVSLAVSAASFLHPFGSPQTDNPCYTFQVLKEFYYQAI

SEQ ID NO: 206 - protein, 130 aa, Alphatorquevirus sp.
GFSASTQAMTSVLDTLYTQNSYWESNLTQFYVLNAKKGSDTTQPLTSNMPTREEFMAKKNTNYNWYTYKAASVKNKLHQMRQTYFEELTSKGPQTTKSEEGYSQHWTTPSTNAYEYHLGMFSAIFLAPDR

SEQ ID NO: 207 - protein, 169 aa, Alphatorquevirus sp.
PVPRFPCAYQDVTYNPLMDKGVGNHIWFQYNTKADTQLIVTGGSCKAHIQDIPLWAAFYGYSDFIESELGPFVDAETVGLVCVICPYTKPPMYNKTNPAMGYVFYDRNFGDGKWTDGRGKIEPYWQVRWRPEMLFQETVMADLVQTGPFSYKDELKNSTLVCKYKFYFT

SEQ ID NO: 208 - protein, 161 aa, Alphatorquevirus sp.
WGGNMMFQQTIKNPCKTDGQPTDSSRHPRGIQVADPEQMGPRWVFHSFDWRRGYLSEKALKRLQEKPLDYDEYFTQPKRPRIFPPTESAEGEFREPEKGSYSEEERSQASAEEQTQEATVLLLKRRLREQQQLQQQLQFLTREMFKTQAGLHLNPMLLNQR

SEQ ID NO: 209 - protein, 765 aa, Alphatorquevirus sp.; identical to SEQ ID NO: 51

SEQ ID NO: 210 - protein, 74 aa, Alphatorquevirus sp.
MAWSWWWQRWRRRRWKPRRRRWRRLRWRRPRRAVRRRRRGRRVRRRRWARRRGRRRRYATRRKRRYRGRRFKKK

SEQ ID NO: 211 - protein, 212 aa, Alphatorquevirus sp.
LVLTQWHPNTMRRCLIKGIVPLVICGHTRWNYNYALHSKDYTEEGRYPHGGALSTTTWSLKVLYDEHLKHHDFWGYPNNQLDLARYKGAKFTFYRHKKTDFIIFFNRKPPFKLNKYSCASYHPGMLMQQRHKILLPSYETKPKGRPKITVRIKPPTLLEDKWYTQQDLCDVNLLQLVVTAADFRHPLCSPQTNTPTTTFQVLKDIYYDTMSI

SEQ ID NO: 212 - protein, 142 aa, Alphatorquevirus sp.
SEPTDSYTSVNNKSTTQTFTNYSNTLENILYTRASYWNSFHATEYLNPNIIYKNGEKLFKEHEDLITWMTQTNNTGFLTKNNTAFGNNSYRPNADKIKKARKTYWNALIGTNDLATNIGQARAERFEYHLGWYSPIFLSRHR

SEQ ID NO: 213 - protein, 167 aa, Alphatorquevirus sp.
SNMNFARAYQDVTYNPNCDRGVNNRVWVQPLTKPTTEFDEKRCKCVVQHLPLWAALYCYQDFVEEELGSSSEILNSCLLVLQCPYTFPPMYDKKLPDKGFVFYDSLFGDGKMSDGRGQVDIFWQQRWYPRLATQMQVMHDITMTGPFSYRDELVSTQLTAKYTFDFM

SEQ ID NO: 214 - protein, 170 aa, Alphatorquevirus sp.
WGGNMISTQIIKNPCKDSGLEPAYPGRQRRDLQIVDPYSMGPQFSFHNWDYRHGLFGQDAIDRVSKQPKDDADYPNPYKRPRYFPPTDQAAQEQEKDFSFLKTAPSNSEESDQEVLQETQVLRFQPEQHKQLHLQLAERQRIGEQLRYLLQQMFKTQANLHLNPYTFTQL

SEQ ID NO: 215 - protein, 666 aa, Betatorquevirus sp.; identical to SEQ ID NO: 58

SEQ ID NO: 216 - protein, 38 aa, Betatorquevirus sp.
MPYYYRRRRYNYRRPRWYGRGWIRRPFRRRFRRKRRVR

SEQ ID NO: 217 - protein, 208 aa, Betatorquevirus sp.
PTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNT

SEQ ID NO: 218 - protein, 128 aa, Betatorquevirus sp.
ISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAG

SEQ ID NO: 219 - protein, 163 aa, Betatorquevirus sp.
TMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFK

SEQ ID NO: 220 - protein, 129 aa, Betatorquevirus sp.
WGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 221 - protein, 673 aa, Gammatorquevirus sp.; identical to SEQ ID NO: 65

SEQ ID NO: 222 - protein, 57 aa, Gammatorquevirus sp.
MPFWWGRRNKFWYGRNYRRKKRRFPKRRKRRFYRRTKYRRPARRRRRRRRKVRRKKK

SEQ ID NO: 223 - protein, 202 aa, Gammatorquevirus sp.
TLIVRQWQPDSIVLCKIKGYDSIIWGAEGTQFQCSTHEMYEYTRQKYPGGGGFGVQLYSLEYLYDQWKLRNNIWTKTNQLKDLCRYLKCVMTFYRHQHIDFVIVYERQPPFEIDKLTYMKYHPYMLLQRKHKIILPSQTTNPRGKLKKKKTIKPPKQMLSKWFFQQQFAKYDLLLIAAAACSLRYPRIGCCNENRMITLYCL

SEQ ID NO: 224 - protein, 92 aa, Gammatorquevirus sp.
NTKFYQDTEWGTTKQAPHYFKPYATINKSMIFVSNYGGKKTEYNIGQWIETDIPGEGNLARYYRSISKEGGYFSPKILQAYQTKVKSVDYKP

SEQ ID NO: 225 - protein, 159 aa, Gammatorquevirus sp.
LPIVLGRYNPAIDDGKGNKIYLQTIMNGHWGLPQKTPDYIIEEVPLWLGFWGYYNYLKQTRTEAIFPLHMFVVQSKYIQTQQTETPNNFWAFIDNSFIQGKNPWDSVITYSEQKLWFPTVAWQLKTINAICESGPYVPKLDNQTYSTWELATHYSFHFK

SEQ ID NO: 226 - protein, 163 aa, Gammatorquevirus sp.
WGGPQISDQPVEDPGNKNKYDVPDTIKEALQIVNPAKNIAATMFHDWDYRRGCITSTAIKRMQQNLPTDSSLESDSDSEPAPKKKRLLPVLHDPQKKTEKINQCLLSLCEESTCQEQETEENILKLIQQQQQQQQKLKHNLLVLIKDLKVKQRLLQLQTGVLE

SEQ ID NO: 227 - protein, 220 aa, synthetic construct
  VARIANT 29: note = Any amino acid
  VARIANT 30: note = Any amino acid
  VARIANT 31: note = Any amino acid
  VARIANT 29..31: note = This region may encompass 0-3 residues
  VARIANT 100: note = Any amino acid
  VARIANT 125: note = Any amino acid
  VARIANT 126: note = Any amino acid
  VARIANT 127: note = Any amino acid
  VARIANT 128: note = Any amino acid
  VARIANT 129: note = Any amino acid
  VARIANT 125..129: note = This region may encompass 1-5 residues
  VARIANT 181: note = Any amino acid
  VARIANT 211: note = Any amino acid
LVLTQWQPNTVRRCYIRGYLPLIICGENXXXTTSRNYATHSDDTIQKGPFGGGMSTTTFSLRVLYDEYQRFMNRWTYSNEDLDLARYLGCKFTFYRHPDXDFIVQYNTNPPFKDTKLTAPSIHPXXXXXGMLMLSKRKILIPSLKTRPKGKHYVKVRIGPPKLFEDKWYTQSDLCDVPLVXLYATAADLQHPFGSPQTDNPCVTFQVLGSXYNKHLSISP

SEQ ID NO: 228 - protein, 172 aa, synthetic construct
  VARIANT 38: note = Any amino acid
  VARIANT 44: note = Any amino acid
  VARIANT 45: note = Any amino acid
  VARIANT 46: note = Any amino acid
  VARIANT 44..46: note = This region may encompass 0-3 residues
  VARIANT 77: note = Any amino acid
  VARIANT 79: note = Any amino acid
  VARIANT 98: note = Any amino acid
  VARIANT 99: note = Any amino acid
  VARIANT 100: note = Any amino acid
  VARIANT 101: note = Any amino acid
  VARIANT 98..101: note = This region may encompass 0-4 residues
SNFEFPGAYTDITYNPLTDKGVGNMVWIQYLTKPDTIXDKTQSXXXKCLIEDLPLWAALYGYVDFCEKETGDSAIIXNXGRVLIRCPYTKPPLYDKTXXXXNKGFVPYSTNFGNGKMPGGSGYVPIYWRARWYPTLFHQKEVLEDIVQSGPFAYKDEKPSTQLVMKYCFNFN

SEQ ID NO: 229 - protein, 258 aa, synthetic construct
  VARIANT 20: note = Any amino acid
  VARIANT 21: note = Any amino acid
  VARIANT 22: note = Any amino acid
  VARIANT 20..22: note = This region may encompass 0-3 residues
  VARIANT 25: note = Any amino acid
  VARIANT 78: note = Any amino acid
  VARIANT 89: note = Any amino acid
  VARIANT 91: note = Any amino acid
  VARIANT 95: note = Any amino acid
  VARIANT 96: note = Any amino acid
  VARIANT 97: note = Any amino acid
  VARIANT 98: note = Any amino acid
  VARIANT 95..98: note = This region may encompass 1-4 residues
  VARIANT 107: note = Any amino acid
  VARIANT 108: note = Any amino acid
  VARIANT 109: note = Any amino acid
  VARIANT 110: note = Any amino acid
  VARIANT 111: note = Any amino acid
  VARIANT 112: note = Any amino acid
  VARIANT 113: note = Any amino acid
  VARIANT 114: note = Any amino acid
  VARIANT 115: note = Any amino acid
  VARIANT 116: note = Any amino acid
  VARIANT 117: note = Any amino acid
  VARIANT 118: note = Any amino acid
  VARIANT 119: note = Any amino acid
  VARIANT 120: note = Any amino acid
  VARIANT 107..120: note = This region may encompass 2-14 residues
  VARIANT 129: note = Any amino acid
  VARIANT 139: note = Any amino acid
  VARIANT 140: note = Any amino acid
  VARIANT 141: note = Any amino acid
  VARIANT 142: note = Any amino acid
  VARIANT 143: note = Any amino acid
  VARIANT 144: note = Any amino acid
  VARIANT 145: note = Any amino acid
  VARIANT 146: note = Any amino acid
  VARIANT 147: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 153: note = Any amino acid
  VARIANT 154: note = Any amino acid
  VARIANT 155: note = Any amino acid
  VARIANT 156: note = Any amino acid
  VARIANT 157: note = Any amino acid
  VARIANT 158: note = Any amino acid
  VARIANT 159: note = Any amino acid
  VARIANT 160: note = Any amino acid
  VARIANT 161: note = Any amino acid
  VARIANT 162: note = Any amino acid
  VARIANT 163: note = Any amino acid
  VARIANT 164: note = Any amino acid
  VARIANT 165: note = Any amino acid
  VARIANT 166: note = Any amino acid
  VARIANT 167: note = Any amino acid
  VARIANT 168: note = Any amino acid
  VARIANT 139..168: note = This region may encompass 0-30 residues
  VARIANT 201: note = Any amino acid
  VARIANT 202: note = Any amino acid
  VARIANT 203: note = Any amino acid
  VARIANT 204: note = Any amino acid
  VARIANT 201..204: note = This region may encompass 0-4 residues
  VARIANT 219: note = Any amino acid
  VARIANT 220: note = Any amino acid
  VARIANT 221: note = Any amino acid
  VARIANT 222: note = Any amino acid
  VARIANT 223: note = Any amino acid
  VARIANT 224: note = Any amino acid
  VARIANT 225: note = Any amino acid
  VARIANT 226: note = Any amino acid
  VARIANT 227: note = Any amino acid
  VARIANT 228: note = Any amino acid
  VARIANT 229: note = Any amino acid
  VARIANT 230: note = Any amino acid
  VARIANT 231: note = Any amino acid
  VARIANT 232: note = Any amino acid
  VARIANT 233: note = Any amino acid
  VARIANT 234: note = Any amino acid
  VARIANT 235: note = Any amino acid
  VARIANT 236: note = Any amino acid
  VARIANT 237: note = Any amino acid
  VARIANT 238: note = Any amino acid
  VARIANT 239: note = Any amino acid
  VARIANT 240: note = Any amino acid
  VARIANT 241: note = Any amino acid
  VARIANT 242: note = Any amino acid
  VARIANT 243: note = Any amino acid
  VARIANT 244: note = Any amino acid
  VARIANT 245: note = Any amino acid
  VARIANT 246: note = Any amino acid
  VARIANT 247: note = Any amino acid
  VARIANT 248: note = Any amino acid
  VARIANT 249: note = Any amino acid
  VARIANT 250: note = Any amino acid
  VARIANT 251: note = Any amino acid
  VARIANT 252: note = Any amino acid
  VARIANT 253: note = Any amino acid
  VARIANT 254: note = Any amino acid
  VARIANT 255: note = Any amino acid
  VARIANT 256: note = Any amino acid
  VARIANT 257: note = Any amino acid
  VARIANT 258: note = Any amino acid
  VARIANT 219..258: note = This region may encompass 0-40 residues
WGGNPISQQVVRNPCKDSGXXXSGXGRQPRSVQVVDPKYMGPEYTFHSWDWRRGLFGEKAIKRMSEQPTDDEIFTGGXPKRPRRDPPTXQXPEEXXXXQKESSSFRXXXXXXXXXXXXXXPWESSSQEXESESQEEEEXXXXXXXXXXXXXXXXXXXXXXXXXXXXXXEQTVQQQLRQQLREQRRLRVQLQLLFQQLLKTXXXXQAGLHINPLLLSQAXXXXXXXXXXXXXXXXXXXXXXXXXXXXXXXXXXXXXXXX

SEQ ID NO: 230 - protein, 214 aa, synthetic construct
  VARIANT 136: note = Any amino acid
  VARIANT 138: note = Any amino acid
  VARIANT 139: note = Any amino acid
  VARIANT 140: note = Any amino acid
  VARIANT 141: note = Any amino acid
  VARIANT 138..141: note = This region may encompass 1-4 residues
  VARIANT 179: note = Any amino acid
LKQWQPSTIRKCKIKGYLPLFQCGKGRISNNYTQYKESIVPHHEPGGGGWSIQQFTLGALYEEHLKLRNWWTKSNDGLPLVRYLGCTIKLYRSEDTDYIVTYQRCYPMTATKLTYLSTQPSRMLMNKHKIIVPSKXTXXXXNKKKKPYKKIFIKPPSQMQNKWYFQQDIANTPLLQLTXTACSLDRMYLSSDSISNNITFTSLNTNFFQNPNFQ

SEQ ID NO: 231 - protein, 187 aa, synthetic construct
  VARIANT 1: note = Any amino acid
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 9: note = Any amino acid
  VARIANT 10: note = Any amino acid
  VARIANT 1..10: note = This region may encompass 4-10 residues
  VARIANT 38: note = Any amino acid
  VARIANT 39: note = Any amino acid
  VARIANT 40: note = Any amino acid
  VARIANT 41: note = Any amino acid
  VARIANT 42: note = Any amino acid
  VARIANT 43: note = Any amino acid
  VARIANT 44: note = Any amino acid
  VARIANT 45: note = Any amino acid
  VARIANT 38..45: note = This region may encompass 1-8 residues
  VARIANT 94: note = Any amino acid
  VARIANT 100: note = Any amino acid
  VARIANT 101: note = Any amino acid
  VARIANT 102: note = Any amino acid
  VARIANT 100..102: note = This region may encompass 1-3 residues
  VARIANT 112: note = Any amino acid
  VARIANT 114: note = Any amino acid
  VARIANT 115: note = Any amino acid
  VARIANT 114..115: note = This region may encompass 0-2 residues
  VARIANT 124: note = Any amino acid
  VARIANT 125: note = Any amino acid
  VARIANT 126: note = Any amino acid
  VARIANT 127: note = Any amino acid
  VARIANT 128: note = Any amino acid
  VARIANT 129: note = Any amino acid
  VARIANT 130: note = Any amino acid
  VARIANT 131: note = Any amino acid
  VARIANT 132: note = Any amino acid
  VARIANT 133: note = Any amino acid
  VARIANT 134: note = Any amino acid
  VARIANT 135: note = Any amino acid
  VARIANT 136: note = Any amino acid
  VARIANT 137: note = Any amino acid
  VARIANT 138: note = Any amino acid
  VARIANT 139: note = Any amino acid
  VARIANT 124..139: note = This region may encompass 3-16 residues
  VARIANT 154: note = Any amino acid
XXXXXXXXXXTPLYFECRYNPFKDKGTGNKVYLVSNNXXXXXXXXTGWDPPTDPDLIIEGFPLWLLLWGWLDWQKKLGKIQNIDTDYILVIQSXYYIPPXXXKLPYYVPLDXDXXFLHGRSPYXXXXXXXXXXXXXXXXPSDKQHWHPKVRFQXETINNIALTGPGTPKLPNQKSIQAHMKYKFYFK

SEQ ID NO: 232 - protein, 163 aa, synthetic construct
  VARIANT 34: note = Any amino acid
  VARIANT 65: note = Any amino acid
  VARIANT 77: note = Any amino acid
  VARIANT 78: note = Any amino acid
  VARIANT 86: note = Any amino acid
  VARIANT 87: note = Any amino acid
  VARIANT 96: note = Any amino acid
  VARIANT 102: note = Any amino acid
  VARIANT 103: note = Any amino acid
  VARIANT 104: note = Any amino acid
  VARIANT 105: note = Any amino acid
  VARIANT 106: note = Any amino acid
  VARIANT 102..106: note = This region may encompass 0-5 residues
  VARIANT 125: note = Any amino acid
  VARIANT 135: note = Any amino acid
  VARIANT 138: note = Any amino acid
  VARIANT 139: note = Any amino acid
  VARIANT 140: note = Any amino acid
  VARIANT 141: note = Any amino acid
  VARIANT 142: note = Any amino acid
  VARIANT 143: note = Any amino acid
  VARIANT 144: note = Any amino acid
  VARIANT 145: note = Any amino acid
  VARIANT 146: note = Any amino acid
  VARIANT 147: note = Any amino acid
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 153: note = Any amino acid
  VARIANT 154: note = Any amino acid
  VARIANT 155: note = Any amino acid
  VARIANT 156: note = Any amino acid
  VARIANT 157: note = Any amino acid
  VARIANT 158: note = Any amino acid
  VARIANT 159: note = Any amino acid
  VARIANT 160: note = Any amino acid
  VARIANT 161: note = Any amino acid
  VARIANT 162: note = Any amino acid
  VARIANT 163: note = Any amino acid
  VARIANT 138..163: note = This region may encompass 0-26 residues
WGGCPAPMETITDPCKQPKYPIPNNLLQTTSLQXPTTPIETYLYKFDERRGLLTKKAAKRIKKDXTTETTLFTDTGXXTSTTLPTXXQTETTQEEXTSEEEXXXXXETLLQQLQQLRRKQKQLRXRILQLLQLLXLLXXXXXXXXXXXXXXXXXXXXXXXXXX

SEQ ID NO: 233 - protein, 203 aa, synthetic construct
  VARIANT 79: note = Any amino acid
  VARIANT 104: note = Any amino acid
  VARIANT 116: note = Any amino acid
  VARIANT 120: note = Any amino acid
  VARIANT 121: note = Any amino acid
  VARIANT 125: note = Any amino acid
  VARIANT 170: note = Any amino acid
TIPLKQWQPESIRKCKIKGYGTLVLGAEGRQFYCYTNEKDEYTPPKAPGGGGFGVELFSLEYLYEQWKARNNIWTKSNXYKDLCRYTGCKITFYRHPTTDFIVXYSRQPPFEIDKXTYMXXHPQXLLLRKHKKIILSKATNPKGKLKKKIKIKPPKQMLNKWFFQKQFAXYGLVQLQAAACBLRYPRLGCCNENRLITLYYLN

SEQ ID NO: 234 - protein, 162 aa, synthetic construct
  VARIANT 12: note = Any amino acid
  VARIANT 20: note = Any amino acid
  VARIANT 23: note = Any amino acid
  VARIANT 30: note = Any amino acid
  VARIANT 84: note = Any amino acid
  VARIANT 90: note = Any amino acid
  VARIANT 95: note = Any amino acid
  VARIANT 105: note = Any amino acid
  VARIANT 113: note = Any amino acid
  VARIANT 154: note = Any amino acid
  VARIANT 156: note = Any amino acid
LPIVVARYNPAXDTGKGNKXWLXSTLNGSXWAPPTTDKDLIIEGLPLWLALYGYWSYJKKVKKDKGILQSHMFVVKSPAIQPLXTATTQXTFYPXIDNSFIQGKXPYDEPJTXNQKKLWYPTLEHQQETINAIVESGPYVPKLDNQKNSTWELXYXYTFYFK

SEQ ID NO: 235 - protein, 177 aa, synthetic construct
  VARIANT 16: note = Any amino acid
  VARIANT 26: note = Any amino acid
  VARIANT 33: note = Any amino acid
  VARIANT 73: note = Any amino acid
  VARIANT 81: note = Any amino acid
  VARIANT 82: note = Any amino acid
  VARIANT 81..82: note = This region may encompass 0-2 residues
  VARIANT 90: note = Any amino acid
  VARIANT 94: note = Any amino acid
  VARIANT 119: note = Any amino acid
  VARIANT 120: note = Any amino acid
  VARIANT 121: note = Any amino acid
  VARIANT 122: note = Any amino acid
  VARIANT 123: note = Any amino acid
  VARIANT 124: note = Any amino acid
  VARIANT 119..124: note = This region may encompass 1-6 residues
  VARIANT 168: note = Any amino acid
  VARIANT 169: note = Any amino acid
  VARIANT 170: note = Any amino acid
  VARIANT 171: note = Any amino acid
  VARIANT 172: note = Any amino acid
  VARIANT 173: note = Any amino acid
  VARIANT 174: note = Any amino acid
  VARIANT 175: note = Any amino acid
  VARIANT 176: note = Any amino acid
  VARIANT 177: note = Any amino acid
  VARIANT 168..177: note = This region may encompass 1-10 residues
WGGPQIPDQPVEDPKXQGTYPVPDTXQQTIQIXNPLKQKPETMFHDWDYRRGIITSTALKRMQENLETDSSFXSDSEETPXXKKKKRLTXELPXPQEETEEIQSCLLSLCEESTCQEEXXXXXXENLQQLIHQQQQQQQQLKHNILKLLSDLKZKQRLLQLQTGILEXXXXXXXXXX

SEQ ID NO: 236-560 - skipped (325 entries)

SEQ ID NO: 561 - protein, 699 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 562 - protein, 688 aa, synthetic construct
MGSSHHHHHHGSDYKDDDDKSGSLEVLFQGPSGMPPYWRQKYYRRRYRPFSWRTRRIIQRRKRWRYRKPRKTYWRRKLRVRKRFYKRKLKKIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHVIRVPSRETRKKRKPYKRVRVGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 563 - protein, 714 aa, synthetic construct
MGSSHHHHHHGSDYKDDDDKSGSLEVLFQGPSGMPPYWRQKYYRRRYRPFSWRTRRIIQRRKRWRYRKPRKTYWRRKLRVRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 564 - protein, 714 aa, synthetic construct
MGSSHHHHHHGSDYKDDDDKSGSLEVLFQGPSGMPPYWRQKYYRRRYRPFSWRTRRIIQRRKRWRYRKPRKTYWRRKLRVRKRFYKRKLKKIVLKQFQPKIIRRCTIFGTICLFQGSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 565 - protein, 688 aa, synthetic construct
MGSSHHHHHHGSDYKDDDDKSGSLEVLFQGPSGMPPYWRQKYYRRRYRPFSWRTRRIIQRRHRWRYRKPRHTYWRRHLRVRHRFYHRHLHHIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHVIRVPSRETRKKRKPYKRVRVGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 566 - protein, 687 aa, synthetic construct
MGSSHHHHHHGSDYKDDDDKSGSLEVLFQGPSGMWGTSNCACAKFQIRRRYARPYRRRHIRRYRRRRRHFRRRRFTTNRRKRFYKRKLKKIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHVIRVPSRETRKKRKPYKRVRVGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 567 - protein, 688 aa, synthetic construct
MGSSHHHHHHGSDYKDDDDKSGSLEVLFQGPSGMPPYWRQKYYRRRYRPFSWRTRRIIQRRKRWRYRKPRKTYWRRKLRVRKRFYKRKLKKIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHAKKWFSRETRKKRKPGFKRLLGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 568 - protein, 699 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRRCTIFGTICLFQGSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 569 - protein, 699 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSPERANNNYIQTIYSYVPDKEPGGGGWTLITESLSSLWEDWEHLKNVWTQSNAGLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 570 - protein, 699 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYGGVTLYFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 571 - protein, 699 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHNCYPMTDTKYTHADSAPNRMLLKKHKHIIPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 572 - protein, 699 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYKRVRVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 573 - protein, 699 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKHVIRVPSRQTRRKKKPYTKIFVKPPPQFENKWYFATDLYKIPLLQIHCTACNLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTTENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSDNISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 574 - protein, 673 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 575 - protein, 673 aa, synthetic construct
MGSSHHHHHHGSDYKDDDDKSGSLEVLFQGPSGMPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTACNLQNPFVKPDKLSNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 576-696 - skipped (121 entries)

SEQ ID NO: 697 - DNA, 20 bp, synthetic construct
  source 1..20: mol_type = other DNA
gaagcccaccaaaagcaatt

SEQ ID NO: 698 - DNA, 21 bp, synthetic construct
  source 1..21: mol_type = other DNA
agttcccgtgtctatagtcga

SEQ ID NO: 699 - DNA, 22 bp, synthetic construct
  source 1..22: mol_type = other DNA
acttcgttacagagtccagggg

SEQ ID NO: 700 - DNA, 20 bp, synthetic construct
  source 1..20: mol_type = other DNA
agcaacaggtaatggaggac

SEQ ID NO: 701 - DNA, 20 bp, synthetic construct
  source 1..20: mol_type = other DNA
tggaagctggggtctttaac

SEQ ID NO: 702 - DNA, 22 bp, synthetic construct
  source 1..22: mol_type = other DNA
tctaccttaggtgcaaagggcc

SEQ ID NO: 703-800 - skipped (98 entries)

SEQ ID NO: 801 - DNA, 156 bp, Torque teno virus; identical to SEQ ID NO: 125
  source 1..156: mol_type = genomic DNA

SEQ ID NO: 802 - DNA, 150 bp, Torque teno virus
  source 1..150: mol_type = genomic DNA
ccgagcgttagcgaggagtgcgaccctaccccctgggcccacttcttcggagccgcgcgctacgccttcggctgcgcgcggcacctcagacccccgctcgtgctgacacgcttgcgcgtgtcagaccacttcgggctcgcgggggtcggg

SEQ ID NO: 803 - DNA, 122 bp, Torque teno virus; identical to SEQ ID NO: 153
  source 1..122: mol_type = genomic DNA

SEQ ID NO: 804 - DNA, 111 bp, Torque teno virus
  source 1..111: mol_type = genomic DNA
cggcccagcggcggcgcgcgcgcttcgcgcgcgcgccggggggctccgcccccccccgcgcatgcgcggggcccccccccgcggggggctccgccccccggtccccccccg

SEQ ID NO: 805 - DNA, 115 bp, Torque teno virus
  source 1..115: mol_type = genomic DNA
cggccgtgcggcggcgcgcgcgcttcgcgcgcgcgccgggggctgccgcccccccccgcgcatgcgcgcggggcccccccccgcggggggctccgccccccggcccccccccccg

SEQ ID NO: 806 - DNA, 104 bp, Torque teno virus; identical to SEQ ID NO: 144
  source 1..104: mol_type = genomic DNA

SEQ ID NO: 807 - DNA, 108 bp, Torque teno virus
  source 1..108: mol_type = genomic DNA
ggcggcggcgcgcgcgctacgcgcgcgcgccggggagctctgcccccccccgcgcatgcgcgcgggtcccccccccgcggggggctccgccccccggtcccccccccg

SEQ ID NO: 808-817 - skipped (10 entries)

SEQ ID NO: 818 - DNA, 36 bp, synthetic construct
  source 1..36: mol_type = other DNA
gcgctkcgcgcgcgcgccggggggctgcgccccccc

SEQ ID NO: 819 - DNA, 36 bp, Torque teno virus; identical to SEQ ID NO: 165
  source 1..36: mol_type = genomic DNA

SEQ ID NO: 820 - DNA, 36 bp, Torque teno virus; identical to SEQ ID NO: 166
  source 1..36: mol_type = genomic DNA

SEQ ID NO: 821 - DNA, 36 bp, Torque teno virus; identical to SEQ ID NO: 167
  source 1..36: mol_type = genomic DNA

SEQ ID NO: 822 - DNA, 36 bp, Torque teno virus; identical to SEQ ID NO: 168
  source 1..36: mol_type = genomic DNA

SEQ ID NO: 823 - DNA, 36 bp, Torque teno virus; identical to SEQ ID NO: 169
  source 1..36: mol_type = genomic DNA

SEQ ID NO: 824 - DNA, 36 bp, Torque teno virus; identical to SEQ ID NO: 169
  source 1..36: mol_type = genomic DNA

SEQ ID NO: 825 - DNA, 36 bp, Torque teno virus; identical to SEQ ID NO: 170
  source 1..36: mol_type = genomic DNA

SEQ ID NO: 826 - DNA, 36 bp, Torque teno virus; identical to SEQ ID NO: 171
  source 1..36: mol_type = genomic DNA

SEQ ID NO: 827 - DNA, 36 bp, Torque teno virus; identical to SEQ ID NO: 172
  source 1..36: mol_type = genomic DNA

SEQ ID NO: 828 - skipped

SEQ ID NO: 829 - protein, 11 aa, synthetic construct
  VARIANT 4: note = Any amino acid
  VARIANT 5: note = Any amino acid
  VARIANT 7: note = Any amino acid
  VARIANT 9: note = Any amino acid
  VARIANT 10: note = Any amino acid
YNPXXDXGXXN

SEQ ID NO: 830 - DNA, 3648 bp, Alphatorquevirus sp.
  source 1..3648: mol_type = genomic DNA
cgtcactaaccacgtgactcccacaggccaaccacagtgtacgtgattcacttcctgggagtggtttacattataatataagcaactgcacttccgaatggctgagttttccacgcccgtccgcagcgagaacaccacggaggggagtccgcgcgtcccgtgggcgggtgccgaaggtgagtttacacaccgcagtcaaggggcaattcgggcacgggactggccgggctatgggcaaggctcttaaaaagctatgtttcttggtaggccgtaccgaaagaaaaggaaactgctactgctaccactgcattctacaccgaaaactagccgggttatgagctggtctaggcctgtacataatgccacaggcattgaaagaaactggtgggagtcctgtcttagatcccacgcaagttcttgtggctgcggtaattttgttaatcatattaatgtactggctaatcggtatggctttgctggttccacggagacgccgggtaatcctcggccgaggcccccggtactgagctccaccaccagcactcctaccgatcaatccagaccagctctaccatggcatggggatactggtggagaaggcgcttctggagaccccgcaggagatggagaacgtggcgccgcagaaggagactacggcccagaagatctagacgcacttttcgacgcactcgacgaagagtaaggaggcgacggtgggggaggcgtgcacgcaggcggggatggcgacgcaggacttatattagagccaggcgacgcaggagacgaaaaagacttgtactgactcagtggcatcccgcagttagaagaaaatgtaaaattacaggctacatgcctatagtatactgtggacatggcagagctagttttaactatgcctggcactctgatgactgtataaaacaaccactaccctttggaggctcactatctacagtgtccttcaacctaaaagtactatttgacgaaaaccaaagaggactaaacaaatggagctacccaaatgaccaactagacctcgccagatacaaaggctgtagactaacattttacagaaaaaaaaacacagactacatagctcaatatgacatatcagaaccttatcaactagacaaatatagctgtgcaaactatcacccctcaaaaatgatgtttgcaaaaaacaaaattttaattcctagctatgatacaaaacctagaggcagacaaagagttagagttagaatagggccccctaaactatttacagacaagtggtacagtcaatcagacttatgcaaggtaaaccttgtgtcacttgcggtttctgcggcttcctttctccacccattcggctcaccacaaactgccaacttttgtgcaaccttccaggtgctgcaaccgttctactaccaggctataggcattagttctacaaaacactcagaagttatagacattttatataagaaaaatacatactggcaaagcaacattacctcttggtttttaactaatgttaaaaacccaaaaaatatgtccacaaaaatgtttgaggacattaatgttaaatcaaacaaagacagtaattatgactggtttccatttaccccatacactacagaaaactattcaaaaattcaaaatgcagctcaagaatactggaaatatttaactagtgaccacccacaagctactaatagcaatgaaggcctagtacaaccatggactaatgccactataaaacaatatgaataccacctcggtatgtttagtcctatatttataggacctaccagagctaaaactaaatttaaaacagcatactttgactgcacttataacccactactagacaaaggaatgggaaacagaatatggtatcaatacgcaaccaaagctgacacacaaatatcaaaaacagggtgctactgcatgttagaagacattccaatatatgcagcattttatggatacgtagactttatagaaatggaaataggtaaaggacaagacattaaagagaacggacttatttgctgcatatgtagatacacagaccccccaatgtacaatgaacaacatccagacatgggatttgtattttataacactaactttggaaatggaaaatggatagatggacggggcgacatacctacttactggatgcaaagatggagacctgttgtattatttcaaactgatgttattagagacttagtagaaactggaccttttagttacaaagatgacctagcaaatacctcactgactatgaaatatgaattctattttacctggggcggaaaccaggcgtaccaccagacaatcaaaaacccttgtaaagacgaaggtaccggaccccatagacagcctagagacgtacaagttacggacccgacaaccgtgggacctgaatatgtgttccacgcgtgggactggagacggggcttccttagcgagcgagctctcagacgcatgttcgaaaaacctctcaactatgatgagtattctaaaaaaccaaaaagacctagaatatttcctccaacagaaacagagtcccgaaaccaagagctcgaagaaagctcgctttcagaggaagaaaagtcgctactctccacagaagagatccagaaagaggagatacagcgacagttcaagcgacagctcaagcgacagctgcgcctcgggcagcagctcaaactcctccaacaacaactcctcaagacgcaagcgggcctgcacctaaaccccctttcatatttcccgcaataaataaagtgtacctgttcccagacagagctccaaaacctaaacccacctctggagactgggaaacagagtatgcagcttgcagtgcctttgacagacccgctagaaccaaccttagctcacccccttactacccaggagtacctactccctggcaagtaaaattcagccttaaatttcaataaagtgcatttttactacagctgggccgtgggagtttcacttgtcggtgtctacctcttaaggtcactaagcactccgagcgcagcgaggagtgcgacccttaaccctgggtcaacgccttcggagccgcgcgctacgccttcggctgcgcgcggcacctcagacccccgctcgtgctgacgcgcttgcgcgcgtcagaccacttcgggctcgcgggggtcgggaactttgctaacagactccgaggtgccattggacacagagtgggcgttcagcaacgaaagtgagtggggccagacttcgccataaggcctttatcttcttgccatttgtcagtataaggggttgccataggcttcggcctcaattttaggccttccggactaccaaaatggccgatttagtgacgtcacggcggccattttaagtaaggcggaagtaactccactatttacaaaatggcggcggagcacttccggcttgcccaaaatggcggcaaaaaacatccgggtcaaaggtcgttaccacgtcacaagtcacgtgggagggtggtgctgtaaacccggaagcaatcctctcacgtggctagtcacgtgactaacacgtcacacccgccattttgttttacaaaatggccgacttccttccgcttttttaaaaataacggctcagcggcggcgcgcgcgctacgcg

SEQ ID NO: 831 - protein, 122 aa, Alphatorquevirus sp.
MSWSRPVHNATGIERNWWESCLRSHASSCGCGNFVNHINVLANRYGFAGSTETPGNPRPRPPVLSSTTSTPTDQSRPALPWHGDTGGEGASGDPAGDGERGAAEGDYGPEDLDALFDALDEE

SEQ ID NO: 832 - protein, 262 aa, Alphatorquevirus sp.
MSWSRPVHNATGIERNWWESCLRSHASSCGCGNFVNHINVLANRYGFAGSTETPGNPRPRPPVLSSTTSTPTDQSRPALPWHGDTGGEGASGDPAGDGERGAAEGDYGPEDLDALFDALDEEQSKTLVKTKVPDPIDSLETYKLRTRQPWDLNMCSTRGTGDGASLASELSDACSKNLSTMMSILKNQKDLEYFLQQKQSPETKSSKKARFQRKKSRYSPQKRSRKRRYSDSSSDSSSDSCASGSSSNSSNNNSSRRKRACT

SEQ ID NO: 833 - protein, 256 aa, Alphatorquevirus sp.
MSWSRPVHNATGIERNWWESCLRSHASSCGCGNFVNHINVLANRYGFAGSTETPGNPRPRPPVLSSTTSTPTDQSRPALPWHGDTGGEGASGDPAGDGERGAAEGDYGPEDLDALFDALDEENRVPKPRARRKLAFRGRKVATLHRRDPERGDTATVQATAQATAAPRAAAQTPPTTTPQDASGPAPKPPFIFPAINKVYLFPDRAPKPKPTSGDWETEYAACSAFDRPARTNLSSPPYYPGVPTPWQVKFSLKFQ

SEQ ID NO: 834 - protein, 178 aa, Alphatorquevirus sp.
MSWSRPVHNATGIERNWWESCLRSHASSCGCGNFVNHINVLANRNRVPKPRARRKLAFRGRKVATLHRRDPERGDTATVQATAQATAAPRAAAQTPPTTTPQDASGPAPKPPFIFPAINKVYLFPDRAPKPKPTSGDWETEYAACSAFDRPARTNLSSPPYYPGVPTPWQVKFSLKFQ

SEQ ID NO: 835 - protein, 733 aa, Alphatorquevirus sp.
MAWGYWWRRRFWRPRRRWRTWRRRRRLRPRRSRRTFRRTRRRVRRRRWGRRARRRGWRRRTYIRARRRRRRKRLVLTQWHPAVRRKCKITGYMPIVYCGHGRASFNYAWHSDDCIKQPLPFGGSLSTVSFNLKVLFDENQRGLNKWSYPNDQLDLARYKGCRLTFYRKKNTDYIAQYDISEPYQLDKYSCANYHPSKMMFAKNKILIPSYDTKPRGRQRVRVRIGPPKLFTDKWYSQSDLCKVNLVSLAVSAASFLHPFGSPQTANFCATFQVLQPFYYQAIGISSTKHSEVIDILYKKNTYWQSNITSWFLTNVKNPKNMSTKMFEDINVKSNKDSNYDWFPFTPYTTENYSKIQNAAQEYWKYLTSDHPQATNSNEGLVQPWTNATIKQYEYHLGMFSPIFIGPTRAKTKFKTAYFDCTYNPLLDKGMGNRIWYQYATKADTQISKTGCYCMLEDIPIYAAFYGYVDFIEMEIGKGQDIKENGLICCICRYTDPPMYNEQHPDMGFVFYNTNFGNGKWIDGRGDIPTYWMQRWRPVVLFQTDVIRDLVETGPFSYKDDLANTSLTMKYEFYFTWGGNQAYHQTIKNPCKDEGTGPHRQPRDVQVTDPTTVGPEYVFHAWDWRRGFLSERALRRMFEKPLNYDEYSKKPKRPRIFPPTETESRNQELEESSLSEEEKSLLSTEEIQKEEIQRQFKRQLKRQLRLGQQLKLLQQQLLKTQAGLHLNPLSYFPQ

SEQ ID NO: 836 - protein, 191 aa, Alphatorquevirus sp.
MAWGYWWRRRFWRPRRRWRTWRRRRRLRPRRSRRTFRRTRRRTIKNPCKDEGTGPHRQPRDVQVTDPTTVGPEYVFHAWDWRRGFLSERALRRMFEKPLNYDEYSKKPKRPRIFPPTETESRNQELEESSLSEEEKSLLSTEEIQKEEIQRQFKRQLKRQLRLGQQLKLLQQQLLKTQAGLHLNPLSYFPQ

SEQ ID NO: 837 - protein, 107 aa, Alphatorquevirus sp.
MAWGYWWRRRFWRPRRRWRTWRRRRRLRPRRSRRTFRRTRRRKQSPETKSSKKARFQRKKSRYSPQKRSRKRRYSDSSSDSSSDSCASGSSSNSSNNNSSRRKRACT

SEQ ID NO: 838 - DNA, 3704 bp, Alphatorquevirus sp.
  source 1..3704: mol_type = genomic DNA
ccccgaagtccgtcactaaccacgtgactcccacaggccaatcagatgctatgtcgtgcacttcctgggctgtgtctacgtcctcatataagtaactgcacttccgaatggctgagttttccacgcccgtccgcagcggcagcaccacggagggtgatccccgcgtcccgtgggcgggtgccggaggtgagtttacacaccgcagtcaaggggcaattcgggcacgggactggccgggctatgggcaaggctcttaaaaagctatgttcttcggtaggtgctggagaaagaaaaggaaagtgcttctgcaagatctgtcaactccaccgaaaaaacctgctatgagtgtgtggcttcctcccatagacaatgttaccgagcgtgagaggagctggctctctagcattcttcagtctcacagagctttttgtgggtgccatgatgctatctatcatcttagcagtctggctgctcgctttaatatgcaaccagggccgtcgccgggtggtgattctaggccgccgcgaccgccactaagacgcctgcccgcgctcccgggtcccagagacccccctagcgacaccaacaaccgcaggtcatggcctactggggatggtggagacggaggcgctggccaaggcgcaggtggaggcgctaccgctaccgaagaagactaccgcgccgaagacctagacgagctgtacgccgccctcgaaggagacgagtaaggaggcgccgcggtagggggtggtacagagggcgacgctactcccgcagacggtacagacgtagatatgtgaggcgaaagagaaagactctagtttggagacagtggcagcctcaaaatatcagaaaatgcaggatcaggggcataattcccatcctgatatgcggacacgggaggggggccagaaactatgcgctccacagcgacgacataaccccccagaacacccccttcgggggaggactgagcaccacctcctggagcctaaaagtgctatatgaccagcacaccaggggactcaacaggtggtctgccagtaacgagagcctagaccttgccagatacaatggctgtagtttcactttctacagagacaaaaagactgactttatagtgacctatgacacctctgctccctacaaactagacaaatacagctcccccagctaccacccagggtccatgatgctcatgacaaaacacaaaatcctgatccccagttttgacacaaaacccaaaggtcctgccaaaattagagtcagaatcaagccccccaaaatgttcttagataaatggtacactcaagacgacctctgttccgttaatcttgtgtcacttgcggttagcgcagcttcctttacacatccgttctgcccaccactaactgacactccttgtgtaacgctgcaggtgttgaaagacttctactacacaaccataggctactcctctaatgcagacaaagtagagtctgtattcactaacactctctacaaacactgctgctactatcagtcctttctcaccactcaatttatagccaaaatcactcgcacaccagatggacaaccagtagccacattctctcctcctacctctttccctggcacaactgtaacaaaaagttccatagaatcatttaaccaatgggtaacttccacaggtacaagtggctggctaacaaatgcaaaccaacactttcatttctgtaactataaaccagatgccacaaagctaaaatggctcagacagtactactttgactgggaaacatacaaattagcagatgtaaagccagacggccttacaccctcagtaaactggtatgagtacagaataggcctctttagtcctattttcctgagccccttcagatctagcagtctagactttcccagagcctaccaggatgtgaactacaaccccctggtagacaaaggagtgggcaacatcatatggttccaatacaacacaaaaccagacacacagctgtcagtacccagctgcaagtgtgtcatagaagacaaacccctatgggcagccttctatggctacagtgactttgtacaacaagagataggagactacacagacgcagaggccgtgggcttcgtctgtgtcatctgtccatacaccaaaccccctctaaaaaacccagacaaccccatgcaagggttcatattctatgacagcctttttggcaatggcaagtggatagatggcacggggcacgtccccctttactggcagagcaggtggaggccagagatgctcttccaagaaaacaccatgagagacatcacactatctgggcccttcagctacaaggacgactataagaactgtgtactgacttgcaaatacaaatttaactttcgattcgggggcaatcttctccacgaacagacgatcagaaacccatgccccacggacggacatcccagtaccggtagacagcctagagacgtacaagtggttgacccgatcaaagtgggcccccggttcgtgttccactcctgggactggcgcagaggctaccttagcccagcagctctcaaaagaattggagagcaaccgctcgattatgaagcttattcgtaccgcccaaagagacctagaatctttcctcccacagaaggagaccagctcgcccgaagtcgagaagaagactcattttcagaggaagaaagtccccatatctcgttcgaagaggggcaggaaccgaaagcccaggcggtacagcagcacctcctccgacacctcagaaagcagcgagaactccgaaagcgactccgagccctgttccaaagcctccaaaagacgcaggcgggtctccacgtaaatccattattattcaaccagcctgcaatcaggttctgatgttcccagagatggggcctaagccagctcccactgcccaagactggcagtgcgaatacgagacatgtaagcactgggatagaccccccagaaagtttctcacagacccccctttctatccctgggcccctactacttacaatgtatctttcaagctaaacttcaaataaactaggccgtgggagtctcacttgtcggtgtctacctcttaaggtcactaagcactccgagcgtcagcgaggagtgcgacccttccccctggtgcaacgccctcggcggccgcgcgctacgccttcggctgcgcgcggcacctcggacccccgctcgtgctgacgcgctcgcgcgcgtcagaccacttcgggctcgcgggggtcgggaaatttgctaaacagactccgagttgccattggacacaggagctgtgaatcagtaacgaaagtgagtggggccagacttcgccataaggcctttatcttcttgccatttgtccgtgaggaggggtcgccaagacgcggaccccgttttcggaccttccgaactaccaaaatggccgattcagtgacgtcacggcagccattttgtgtaagcaccgcccaggacagacgtcacagttcaaaggtcatcctcgagcggaacttacagaaaatggcggtcaattgcttccgggtcaaaggtcacgcctacgtcataagtcacgtggtggaggctactgcgcatacacggaagtaggccccgccacgtgaccgaccacgtgggtgctgcgtcacggccgccattttgtatcacaaaatggccgacttccttcctctttttcaaa

SEQ ID NO: 839 - protein, 128 aa, Alphatorquevirus sp.
MSVWLPPIDNVTERERSWLSSILQSHRAFCGCHDAIYHLSSLAARFNMQPGPSPGGDSRPPRPPLRRLPALPGPRDPPSDTNNRRSWPTGDGGDGGAGQGAGGGATATEEDYRAEDLDELYAALEGDE

SEQ ID NO: 840 - protein, 272 aa, Alphatorquevirus sp.
MSVWLPPIDNVTERERSWLSSILQSHRAFCGCHDAIYHLSSLAARFNMQPGPSPGGDSRPPRPPLRRLPALPGPRDPPSDTNNRRSWPTGDGGDGGAGQGAGGGATATEEDYRAEDLDELYAALEGDERSETHAPRTDIPVPVDSLETYKWLTRSKWAPGSCSTPGTGAEATLAQQLSKELESNRSIMKLIRTAQRDLESFLPQKETSSPEVEKKTHFQRKKVPISRSKRGRNRKPRRYSSTSSDTSESSENSESDSEPCSKASKRRRRVST

SEQ ID NO: 841 - protein, 265 aa, Alphatorquevirus sp.
MSVWLPPIDNVTERERSWLSSILQSHRAFCGCHDAIYHLSSLAARFNMQPGPSPGGDSRPPRPPLRRLPALPGPRDPPSDTNNRRSWPTGDGGDGGAGQGAGGGATATEEDYRAEDLDELYAALEGDERRPARPKSRRRLIFRGRKSPYLVRRGAGTESPGGTAAPPPTPQKAARTPKATPSPVPKPPKDAGGSPRKSIIIQPACNQVLMFPEMGPKPAPTAQDWQCEYETCKHWDRPPRKFLTDPPFYPWAPTTYNVSFKLNFK

SEQ ID NO: 842 - protein, 762 aa, Alphatorquevirus sp.
MAYWGWWRRRRWPRRRWRRYRYRRRLPRRRPRRAVRRPRRRRVRRRRGRGWYRGRRYSRRRYRRRYVRRKRKTLVWRQWQPQNIRKCRIRGIIPILICGHGRGARNYALHSDDITPQNTPFGGGLSTTSWSLKVLYDQHTRGLNRWSASNESLDLARYNGCSFTFYRDKKTDFIVTYDTSAPYKLDKYSSPSYHPGSMMLMTKHKILIPSFDTKPKGPAKIRVRIKPPKMFLDKWYTQDDLCSVNLVSLAVSAASFTHPFCPPLTDTPCVTLQVLKDFYYTTIGYSSNADKVESVFTNTLYKHCCYYQSFLTTQFIAKITRTPDGQPVATFSPPTSFPGTTVTKSSIESFNQWVTSTGTSGWLTNANQHFHFCNYKPDATKLKWLRQYYFDWETYKLADVKPDGLTPSVNWYEYRIGLFSPIFLSPFRSSSLDFPRAYQDVNYNPLVDKGVGNIIWFQYNTKPDTQLSVPSCKCVIEDKPLWAAFYGYSDFVQQEIGDYTDAEAVGFVCVICPYTKPPLKNPDNPMQGFIFYDSLFGNGKWIDGTGHVPLYWQSRWRPEMLFQENTMRDITLSGPFSYKDDYKNCVLTCKYKFNFRFGGNLLHEQTIRNPCPTDGHPSTGRQPRDVQVVDPIKVGPRFVFHSWDWRRGYLSPAALKRIGEQPLDYEAYSYRPKRPRIFPPTEGDQLARSREEDSFSEEESPHISFEEGQEPKAQAVQQHLLRHLRKQRELRKRLRALFQSLQKTQAGLHVNPLLFNQPAIRF

SEQ ID NO: 843 - protein, 199 aa, Alphatorquevirus sp.
MAYWGWWRRRRWPRRRWRRYRYRRRLPRRRPRRAVRRPRRRRTIRNPCPTDGHPSTGRQPRDVQVVDPIKVGPRFVFHSWDWRRGYLSPAALKRIGEQPLDYEAYSYRPKRPRIFPPTEGDQLARSREEDSFSEEESPHISFEEGQEPKAQAVQQHLLRHLRKQRELRKRLRALFQSLQKTQAGLHVNPLLFNQPAIRF

SEQ ID NO: 844 - protein, 110 aa, Alphatorquevirus sp.
MAYWGWWRRRRWPRRRWRRYRYRRRLPRRRPRRAVRRPRRRRKETSSPEVEKKTHFQRKKVPISRSKRGRNRKPRRYSSTSSDTSESSENSESDSEPCSKASKRRRRVST

SEQ ID NO: 845 - DNA, 3653 bp, Alphatorquevirus sp.
  source 1..3653: mol_type = genomic DNA
  misc_difference 2949: note = a, c, t, g, unknown or other
  misc_difference 2950: note = a, c, t, g, unknown or other
  misc_difference 2951: note = a, c, t, g, unknown or other
  misc_difference 2952: note = a, c, t, g, unknown or other
  misc_difference 2953: note = a, c, t, g, unknown or other
  misc_difference 2954: note = a, c, t, g, unknown or other
  misc_difference 2955: note = a, c, t, g, unknown or other
  misc_difference 2956: note = a, c, t, g, unknown or other
  misc_difference 2957: note = a, c, t, g, unknown or other
  misc_difference 2958: note = a, c, t, g, unknown or other
  misc_difference 2959: note = a, c, t, g, unknown or other
  misc_difference 2960: note = a, c, t, g, unknown or other
  misc_difference 2961: note = a, c, t, g, unknown or other
  misc_difference 2962: note = a, c, t, g, unknown or other
  misc_difference 2963: note = a, c, t, g, unknown or other
  misc_difference 2964: note = a, c, t, g, unknown or other
  misc_difference 2965: note = a, c, t, g, unknown or other
  misc_difference 2966: note = a, c, t, g, unknown or other
ccaaccagagtctatgtcgtgcacttcctgggcatggtctacgtaataatataaagcggtgcacttccgaatggctgagttttccacgcccgtccgcagcgagatcgcgacggaggagcgatcgagcgtcccgagggcgggtgccggaggtgagtttacacaccgcagtcaaggggcaattcgggctcgggactggccgggctatgggcaaggctcttaaaaagccatgtttctcggtaaactttacaggcagaaaaggaaactgctactgcagcctgtgcgtgctccacagacgccatcttccatgagctctacctggcgagtgccccgcggcgatgtctccgcccgcgagctatgttggtaccgctcagttcgagagagccacgatgctttttgtggctgtcgtgatcctgtttttcatctttctcgtctggctgcacgttctaaccatcagggacctccgacgccccccacggacgagcgcccgtcggcgtctaccccagtgaggcgcctgctgccgctgccctcctaccccggcgagggtccccaggctagatggcctggtggggatggagaaggcgctggtggcgcccgcggaggcgctggagatggcggcgcccgcgcaggcgaagaagagtaccggcccgaagacctcgacgagctgttcgacgctatcgaacaagaacagtaaggagacggaggcgagggtggcggaggggctacaggcgccgttacagactgagacgctaccgtagaaggggcaggcgacgcaaaaaaatagtactgactcagtggaacccccagactgtcagaaagtgctttatcagaggactgatgccagtactatgggcgggcatgggcacggggggccacaactacgccgtccgctcagatgactttgtggtagacagaggcttcgggggctccttcgccacagaaactttctccctgagggtcctctttgaccagtaccagagaggatttaataggtggtctcacaccaacgaagacctagacctggcccgctacacgggctgcaaatggacattttacagacaccaagacacagactttatagtgtactttacaaacaatccccccatgaaaaccaaccagcacacagcccctctcacaactccaggcatgctcatgaggagcaagtataaaatactagtgcccagttttaaaacaagaccaaagggcagaaaaacagtgtcagtgagagttagaccccccaaactgtttcaggacaaatggtatactcaacaggacctctgtccagtacccctcgtccaactgaacgtgaccgcagcggatttcacacatccgttcggctcaccactaactgacacgccttgcataagattccaagttttagggaacttatacaacaagtgcctaaatatagatcttccgcaatttgatgaggacggtgagatactcacttcaacaccttataacagagaaaacaaagaagatcttaaaaagctttataaaactctatttgtagatgaacacgcaggcaattattggcagacattcttaaccaacacaatggtaaagtcacacatagatgcaaaccaagcaaagacatacgatcaagaaaaaactgctgcagaacaaggtaaagaccccttcccaacaaacccaccaaaagaccaattcactacctggaacaagaaactagtagaccctagagacagcaactttctctttgccacatatcacccaaaaaacattaaaaaagctataaaaaccatgagagacaacaactttgctctcaccacaggcaaaaatgacatatatggagactacaccgcggcctacaccagaaacacccacatgctagactactacctaggcttttatagccccatatttctttccagcggtaggtccaacacagagttctggaccgcctacagagacatagtatataatcccctcttagacaaaggcacaggcaacatgatctggttccaatatcacacaaaaacagacaatatatacaaaaaaccagagtgccactgggagatactagacatgcccctgtgggccctctgcaacgggtatgtagagtacctagagagccaaataaagtacggggacatcctagtagagggcaaagtcctcatcagatgcccctacaccaaacccgcactggtagaccccaataacagcctagctggttacgtggtattcaacaccaccttcggccagggaaaatggatagatggcaaaggctacatccccctacacgagaggagcaagtggtacgtcatgctcagataccagaccgacgtactccatgacatagtgacttgtggaccctggcagtacagagacgataacaaaaactctcagctaatagccaagtacagattcaagttctactggggaggtaacatggtacattctcaggtcatcagaaacccgtgcaaagacacccaagtatccggaccccgtcgacagcctcgcgaagtacaagtcgttgacccgcaactcattacgccgccgtgggtcctccactcgttcgaccagagacgaggaatgtttactgcaggagctatcaaacgtctgctcaagcaaccaatacctggcgagtatgctcctacaccactcagggtcccgctcctctttccctcctcagagttccagcgagagggagaagatgcagaaagcggctcaggttcaccacccaagagaccgcgactctggcaggaagaggccaaccagacgcaaacggagtcctcggaggggccggcggagacgacgagggagctcctcgagcgaaagctcagagagcagcgagtcctcaacctccaactccagcatgtcgcagtacaactcgccaaaacccaagcgaacctccacataaaccccctattatactcccagccttaaacaaagtgtatctattcccccctgacaagcccactcccatacagnnnnnnnnnnnnnnnnnnaacacagagttcgaagcctgccaggccttcgacagaccacctagaaaatacctctcagacacacctacctacccttggctccccgtccccaatcctgaaataaaggtcagctttaagctcggtttcaaatcttacaaggccgtgggagtttcactggtcggtgtctacctcttaaggtcactaagcactccgagcgtcagcgaggagtgcgacccttccccctggtgcaacgccctcggcggccgcgcgctacgccttcggctgcgcgcggcacctcggacccccgctcgtgctgacgcgctcgcgcgcgtcagaccacttcgggctcgcgggggtcgggaattttgctaaacagactccgagttgccattggacactgtagctgtgaatcagtaacgaaagtgagtggggccagacttcgccataaggcctttatcttcttgccattggtccgtgtagggggtcgccataggcttcgggttcggttttaggccttccggactacaaaaatggcggatttagtgacgtcacggccgccattttaagtaggtgccgtccaggactgctgttccgggtcacagggcatcctcggcggaacttacacaaaatggcggtcaaaaacatccgggtcaaaggtcgcagctacgtcataagtcacgtgcaggggtcctgctgcgtcatatgcgg

SEQ ID NO: 846 - protein, 128 aa, Alphatorquevirus sp.
MSSTWRVPRGDVSARELCWYRSVRESHDAFCGCRDPVFHLSRLAARSNHQGPPTPPTDERPSASTPVRRLLPLPSYPGEGPQARWPGGDGEGAGGARGGAGDGGARAGEEEYRPEDLDELFDAIEQEQ

SEQ ID NO: 847 - protein, 279 aa, Alphatorquevirus sp.
MSSTWRVPRGDVSARELCWYRSVRESHDAFCGCRDPVFHLSRLAARSNHQGPPTPPTDERPSASTPVRRLLPLPSYPGEGPQARWPGGDGEGAGGARGGAGDGGARAGEEEYRPEDLDELFDAIEQEQSSETRAKTPKYPDPVDSLAKYKSLTRNSLRRRGSSTRSTRDEECLLQELSNVCSSNQYLASMLLHHSGSRSSFPPQSSSEREKMQKAAQVHHPRDRDSGRKRPTRRKRSPRRGRRRRRGSSSSESSESSESSTSNSSMSQYNSPKPKRTST

SEQ ID NO: 848 - protein, 378 aa, Alphatorquevirus sp.
  VARIANT 257: note = Any amino acid
  VARIANT 258: note = Any amino acid
  VARIANT 259: note = Any amino acid
  VARIANT 260: note = Any amino acid
  VARIANT 261: note = Any amino acid
  VARIANT 262: note = Any amino acid
MSSTWRVPRGDVSARELCWYRSVRESHDAFCGCRDPVFHLSRLAARSNHQGPPTPPTDERPSASTPVRRLLPLPSYPGEGPQARWPGGDGEGAGGARGGAGDGGARAGEEEYRPEDLDELFDAIEQEQSYQTSAQATNTWRVCSYTTQGPAPLSLLRVPARGRRCRKRLRFTTQETATLAGRGQPDANGVLGGAGGDDEGAPRAKAQRAASPQPPTPACRSTTRQNPSEPPHKPPIILPALNKVYLFPPDKPTPIQXXXXXXNTEFEACQAFDRPPRKYLSDTPTYPWLPVPNPEIKVSFKLGFKSYKAVGVSLVGVYLLRSLSTPSVSEECDPSPWCNALGGRALRLRLRAAPRTPARADALARVRPLRARGGREFC

SEQ ID NO: 849 - protein, 269 aa, Alphatorquevirus sp.
  VARIANT 148: note = Any amino acid
  VARIANT 149: note = Any amino acid
  VARIANT 150: note = Any amino acid
  VARIANT 151: note = Any amino acid
  VARIANT 152: note = Any amino acid
  VARIANT 153: note = Any amino acid
MSSTWRVPRGDVSARELCWSYQTSAQATNTWRVCSYTTQGPAPLSLLRVPARGRRCRKRLRFTTQETATLAGRGQPDANGVLGGAGGDDEGAPRAKAQRAASPQPPTPACRSTTRQNPSEPPHKPPIILPALNKVYLFPPDKPTPIQXXXXXXNTEFEACQAFDRPPRKYLSDTPTYPWLPVPNPEIKVSFKLGFKSYKAVGVSLVGVYLLRSLSTPSVSEECDPSPWCNALGGRALRLRLRAAPRTPARADALARVRPLRARGGREFC

SEQ ID NO: 850 - protein, 782 aa, Alphatorquevirus sp.
MAWWGWRRRWWRPRRRWRWRRPRRRRRVPARRPRRAVRRYRTRTVRRRRRGWRRGYRRRYRLRRYRRRGRRRKKIVLTQWNPQTVRKCFIRGLMPVLWAGMGTGGHNYAVRSDDFVVDRGFGGSFATETFSLRVLFDQYQRGFNRWSHTNEDLDLARYTGCKWTFYRHQDTDFIVYFTNNPPMKTNQHTAPLTTPGMLMRSKYKILVPSFKTRPKGRKTVSVRVRPPKLFQDKWYTQQDLCPVPLVQLNVTAADFTHPFGSPLTDTPCIRFQVLGNLYNKCLNIDLPQFDEDGEILTSTPYNRENKEDLKKLYKTLFVDEHAGNYWQTFLTNTMVKSHIDANQAKTYDQEKTAAEQGKDPFPTNPPKDQFTTWNKKLVDPRDSNFLFATYHPKNIKKAIKTMRDNNFALTTGKNDIYGDYTAAYTRNTHMLDYYLGFYSPIFLSSGRSNTEFWTAYRDIVYNPLLDKGTGNMIWFQYHTKTDNIYKKPECHWEILDMPLWALCNGYVEYLESQIKYGDILVEGKVLIRCPYTKPALVDPNNSLAGYVVFNTTFGQGKWIDGKGYIPLHERSKWYVMLRYQTDVLHDIVTCGPWQYRDDNKNSQLIAKYRFKFYWGGNMVHSQVIRNPCKDTQVSGPRRQPREVQVVDPQLITPPWVLHSFDQRRGMFTAGAIKRLLKQPIPGEYAPTPLRVPLLFPSSEFQREGEDAESGSGSPPKRPRLWQEEANQTQTESSEGPAETTRELLERKLREQRVLNLQLQHVAVQLAKTQANLHINPLLYSQP

SEQ ID NO: 851 - skipped

SEQ ID NO: 852 - protein, 204 aa, Alphatorquevirus sp.
MAWWGWRRRWWRPRRRWRWRRPRRRRRVPARRPRRAVRRYRTRTVIRNPCKDTQVSGPRRQPREVQVVDPQLITPPWVLHSFDQRRGMFTAGAIKRLLKQPIPGEYAPTPLRVPLLFPSSEFQREGEDAESGSGSPPKRPRLWQEEANQTQTESSEGPAETTRELLERKLREQRVLNLQLQHVAVQLAKTQANLHINPLLYSQP

SEQ ID NO: 853 - protein, 148 aa, Alphatorquevirus sp.
MAWWGWRRRWWRPRRRWRWRRPRRRRRVPARRPRRAVRRYRTRTELSNVCSSNQYLASMLLHHSGSRSSFPPQSSSEREKMQKAAQVHHPRDRDSGRKRPTRRKRSPRRGRRRRRGSSSSESSESSESSTSNSSMSQYNSPKPKRTST

SEQ ID NO: 854 - DNA, 3742 bp, Alphatorquevirus sp.
  source 1..3742: mol_type = genomic DNA
aaagtgctacgtcactaaccacgtgacacccacaggccaaccgaatgctatgtcgtgcacttcctgggccgggtctacgtcctcatataactacctgcacttccgaatggctgagttttccacgcccgtccgcagcggtgaagccacggagggagatcagcgcgtcccgagggcgggtgccgaaggtgagtttacacaccgaagtcaaggggcaattcgggctcgggactggccgggctatgggcaaggctctgaaaaaagcatgtttattggcaggcattacagaaagaaaagggcgctgccactgtgtgctgtgcgatcaacaaagaaggcttgcaaactactaatagtaatgtggaccccacctcgcaatgaccaacagtaccttaactggcaatggtactcaagtatacttagctcccacgctgctatgtgcgggtgtcccgacgttgttgctcattttaatcatcttgcttctgtgcttcgcgccccgcaaaatccacccccacccggtccccagcgaaacctgcccctccgacggctgccggctctcccggctgcgccagaggcgcccggagatagagcaccatggcctatggctggtggcgccggaggagaagacggtggcgcaggtggagacgcagaccatggaggcgccgctggaggaccagaagacgcagacctgttagacgccgtggccgccgcagaaacgtaaggagacgccgcagaggagggaggtggaggaggaggtacaggagatggaaaagaaagggcagacgcagaaaaaaagctaaaataataataagacaatggcaacctaactacagaaggagatgtaacatagtaggctatattcctgtactgatatgtggcgaaaatactgtcagcagaaactatgccacacactcagacgatactaactacccaggaccctttggggggggtatgactacagacaaatttaccttaagaattctgtatgacgagtacaaaaggtttatgaactattggacagcatctaatgaagacctagacctctgtagatatctaggagtaaacctgtacttttttagacacccagaagtagactttattataaaaataaataccatgcccccttttctagacacagaactaacagctcctagcatacacccaggaatgctagccttagacaaaagagcaagatggatacctagcttaaaatctagaccaggaaaaaaacactatattaaaataagagtaggggcgcctaaaatgttcacagataaatggtacccccaaacagatctttgtgacatggtgctgctaactgtctatgcaaccgcagcggatatgcaatatccgttcggctcaccactaactgactctgtggttgtgaacttccaggttctgcaatccatgtatgatgaaaccattagcatattaccagatcaaaaggagaaaagaataacgctgctcactagtatagccttttataacaccacacaaactatagcccaattaaagccatttatagatgcaggcaatatgacttcaactacaacagcaacaacatggggatcatacataaacacaaccaaatttaatacagcagccactacaacatacacatacccaggcagtactacaactacagtaactatgttaacttgtaatgactcctggtacagaggaacagtatataacgaccaaattaaaaatttaccaaaggaagcagctcaattatacttaaaagcaacaaaaaccttactaggaaacaccttcacaaatgacgaccacacactagaataccatggaggactgtacagctcaatttggctgtcccccggcagatcttactttgaaacaccaggagcatacacagacataaaatacaacccatttacagacagaggagaaggaaacatgctatggatagactggctaagcaaaaaaaatatgaactatgacaaactacaaagtaaatgtttaatatcagacctacctttatgggcagcagcatatggatatttagaattttgtgcaaaaagtacaggagaccaaaatatacacatgaatgccagactactaataagaagtccctttacagacccccaactactagtacacacaaaccccacaaaaggctttgttccctactctttaaactttggaaatggtaaaatgccaggaggtagtagtaatgttcctattagaatgagagctaaatggtatccaacattgtttcaccagcaagaagtactagaggccttagcacagtcaggcccctttgcataccactcagacattaaaaaagtatctctgggtatgaaataccgttttaagtggatctggggtggaaaccccgttcgccaacaggttgttagaaatccctgcaaagactcccactcctcggtcaatagagtccctagaagcttacaaatcgttgacccgaaatacaactcaccggaactcacattccatacgtgggacttcagacgtggcctctttggccagaaagctattgagagaatgcaacaacaaccaacaactactgacattttttcagcaggccgcaagagacccaggagggacaccgaggtgtaccactccagccaagaaggggagcaaaaagaaagcttacttttccccccagtcaagctcctcagacgagtccccccgtgggaagactcgcagcaggaggaaagcgggtcgcaaagctcagaggaagagacgcagaccgtctcccagcagctcaagcagcagctgcagcaacagcgaatcctgggagtcaaactcatactcctgttcaaccaagtccaaaaaatccaacaaaatcaagatatcaaccctaccttgttaccaagggggggggatctagcatccttatttcaaatagcaccataaacatgtttggagaccccaaaccttacaacccttccagtaatgactggaaagaggagtatgaggcctgtagaatatgggacagacccccaagaggcaatctaagagacacccccttttacccctgggcccccaaagaaaaccagtaccgtgtaaactttaaacttggatttcaataaagctaggccgtgggactttcacttgtcggtgtctgcttataaaagtaaccaagcactccgagcgaagcgaggagtgcgacccttgggggctcaacgacttcggagccgcgcgttaagccttcggctgcgcgcggcacctcagacccccgctcgtgctgacacgcttgcgcgtgtcagaccacttcgggctcgcgggggtcgggaaatttattaaacagactccgagttgccattggacacagtagtctatgaacagcaacgaaagtgagtggggccagacttcgccataaggcctttatcttcttgccatttgtcagtatagagggtcgccataggcttcggtctccattttaacctgtaaaaactaccaaaatggccgttccagtgacgtgacagccgccattttaagtagctgacgtcaaggattgacgtaaaggttaaaggtcatcctcggcggaagctacacaaaatggtggacaacatcttccgggtcaaaggtcgtgcacacgtcaaaagtcacgtggtggggacccgctgtaacccggaagtaggccccgtcacgtgatttgtcacgtgtgtacacgtcacagccgccattttgttttacaaaatggctgacttccttcctcttttttgaaaaaaggcgccaaaaaaggctccgccccccggcccccccc

SEQ ID NO: 855 - protein, 120 aa, Alphatorquevirus sp.
MWTPPRNDQQYLNWQWYSSILSSHAAMCGCPDVVAHFNHLASVLRAPQNPPPPGPQRNLPLRRLPALPAAPEAPGDRAPWPMAGGAGGEDGGAGGDADHGGAAGGPEDADLLDAVAAAET

SEQ ID NO: 856 - protein, 286 aa, Alphatorquevirus sp.
MWTPPRNDQQYLNWQWYSSILSSHAAMCGCPDVVAHFNHLASVLRAPQNPPPPGPQRNLPLRRLPALPAAPEAPGDRAPWPMAGGAGGEDGGAGGDADHGGAAGGPEDADLLDAVAAAETLLEIPAKTPTPRSIESLEAYKSLTRNTTHRNSHSIRGTSDVASLARKLLRECNNNQQLLTFFQQAARDPGGTPRCTTPAKKGSKKKAYFSPQSSSSDESPRGKTRSRRKAGRKAQRKRRRPSPSSSSSSCSNSESWESNSYSCSTKSKKSNKIKISTLPCYQGGGI

SEQ ID NO: 857 - protein, 289 aa, Alphatorquevirus sp.
MWTPPRNDQQYLNWQWYSSILSSHAAMCGCPDVVAHFNHLASVLRAPQNPPPPGPQRNLPLRRLPALPAAPEAPGDRAPWPMAGGAGGEDGGAGGDADHGGAAGGPEDADLLDAVAAAETPQETQEGHRGVPLQPRRGAKRKLTFPPSQAPQTSPPVGRLAAGGKRVAKLRGRDADRLPAAQAAAAATANPGSQTHTPVQPSPKNPTKSRYQPYLVTKGGGSSILISNSTINMFGDPKPYNPSSNDWKEEYEACRIWDRPPRGNLRDTPFYPWAPKENQYRVNFKLGFQ

SEQ ID NO: 858 - protein, 196 aa, Alphatorquevirus sp.
MWTPPRNDQQYLNWQWYSSILSSHAAMPQETQEGHRGVPLQPRRGAKRKLTFPPSQAPQTSPPVGRLAAGGKRVAKLRGRDADRLPAAQAAAAATANPGSQTHTPVQPSPKNPTKSRYQPYLVTKGGGSSILISNSTINMFGDPKPYNPSSNDWKEEYEACRIWDRPPRGNLRDTPFYPWAPKENQYRVNFKLGFQ

SEQ ID NO: 859 - protein, 766 aa, Alphatorquevirus sp.
MAYGWWRRRRRRWRRWRRRPWRRRWRTRRRRPVRRRGRRRNVRRRRRGGRWRRRYRRWKRKGRRRKKAKIIIRQWQPNYRRRCNIVGYIPVLICGENTVSRNYATHSDDTNYPGPFGGGMTTDKFTLRILYDEYKRFMNYWTASNEDLDLCRYLGVNLYFFRHPEVDFIIKINTMPPFLDTELTAPSIHPGMLALDKRARWIPSLKSRPGKKHYIKIRVGAPKMFTDKWYPQTDLCDMVLLTVYATAADMQYPFGSPLTDSVVVNFQVLQSMYDETISILPDQKEKRITLLTSIAFYNTTQTIAQLKPFIDAGNMTSTTTATTWGSYINTTKFNTAATTTYTYPGSTTTTVTMLTCNDSWYRGTVYNDQIKNLPKEAAQLYLKATKTLLGNTFTNDDHTLEYHGGLYSSIWLSPGRSYFETPGAYTDIKYNPFTDRGEGNMLWIDWLSKKNMNYDKLQSKCLISDLPLWAAAYGYLEFCAKSTGDQNIHMNARLLIRSPFTDPQLLVHTNPTKGFVPYSLNFGNGKMPGGSSNVPIRMRAKWYPTLFHQQEVLEALAQSGPFAYHSDIKKVSLGMKYRFKWIWGGNPVRQQVVRNPCKDSHSSVNRVPRSLQIVDPKYNSPELTFHTWDFRRGLFGQKAIERMQQQPTTTDIFSAGRKRPRRDTEVYHSSQEGEQKESLLFPPVKLLRRVPPWEDSQQEESGSQSSEEETQTVSQQLKQQLQQQRILGVKLILLFNQVQKIQQNQDINPTLLPRGGDLASLFQIAP

SEQ ID NO: 860 - protein, 216 aa, Alphatorquevirus sp.
MAYGWWRRRRRRWRRWRRRPWRRRWRTRRRRPVRRRGRRRNVVRNPCKDSHSSVNRVPRSLQIVDPKYNSPELTFHTWDFRRGLFGQKAIERMQQQPTTTDIFSAGRKRPRRDTEVYHSSQEGEQKESLLFPPVKLLRRVPPWEDSQQEESGSQSSEEETQTVSQQLKQQLQQQRILGVKLILLFNQVQKIQQNQDINPTLLPRGGDLASLFQIAP

SEQ ID NO: 861 - protein, 143 aa, Alphatorquevirus sp.
MAYGWWRRRRRRWRRWRRRPWRRRWRTRRRRPVRRRGRRRNAARDPGGTPRCTTPAKKGSKKKAYFSPQSSSSDESPRGKTRSRRKAGRKAQRKRRRPSPSSSSSSCSNSESWESNSYSCSTKSKKSNKIKISTLPCYQGGGI

SEQ ID NO: 862 - DNA, 3553 bp, Alphatorquevirus sp.
  source 1..3553: mol_type = genomic DNA
  misc_difference 1842: note = a, c, t, g, unknown or other
  misc_difference 3125: note = a, c, t, g, unknown or other
  misc_difference 3126: note = a, c, t, g, unknown or other
  misc_difference 3127: note = a, c, t, g, unknown or other
  misc_difference 3128: note = a, c, t, g, unknown or other
  misc_difference 3129: note = a, c, t, g, unknown or other
  misc_difference 3130: note = a, c, t, g, unknown or other
  misc_difference 3131: note = a, c, t, g, unknown or other
  misc_difference 3132: note = a, c, t, g, unknown or other
  misc_difference 3133: note = a, c, t, g, unknown or other
  misc_difference 3134: note = a, c, t, g, unknown or other
  misc_difference 3135: note = a, c, t, g, unknown or other
  misc_difference 3136: note = a, c, t, g, unknown or other
  misc_difference 3137: note = a, c, t, g, unknown or other
  misc_difference 3138: note = a, c, t, g, unknown or other
  misc_difference 3139: note = a, c, t, g, unknown or other
  misc_difference 3140: note = a, c, t, g, unknown or other
  misc_difference 3141: note = a, c, t, g, unknown or other
  misc_difference 3142: note = a, c, t, g, unknown or other
  misc_difference 3143: note = a, c, t, g, unknown or other
  misc_difference 3144: note = a, c, t, g, unknown or other
  misc_difference 3145: note = a, c, t, g, unknown or other
  misc_difference 3146: note = a, c, t, g, unknown or other
  misc_difference 3147: note = a, c, t, g, unknown or other
  misc_difference 3148: note = a, c, t, g, unknown or other
  misc_difference 3149: note = a, c, t, g, unknown or other
  misc_difference 3150: note = a, c, t, g, unknown or other
  misc_difference 3151: note = a, c, t, g, unknown or other
  misc_difference 3152: note = a, c, t, g, unknown or other
  misc_difference 3153: note = a, c, t, g, unknown or other
  misc_difference 3154: note = a, c, t, g, unknown or other
  misc_difference 3155: note = a, c, t, g, unknown or other
  misc_difference 3156: note = a, c, t, g, unknown or other
  misc_difference 3157: note = a, c, t, g, unknown or other
  misc_difference 3158: note = a, c, t, g, unknown or other
  misc_difference 3159: note = a, c, t, g, unknown or other
  misc_difference 3160: note = a, c, t, g, unknown or other
  misc_difference 3161: note = a, c, t, g, unknown or other
  misc_difference 3162: note = a, c, t, g, unknown or other
  misc_difference 3163: note = a, c, t, g, unknown or other
  misc_difference 3164: note = a, c, t, g, unknown or other
  misc_difference 3165: note = a, c, t, g, unknown or other
  misc_difference 3166: note = a, c, t, g, unknown or other
  misc_difference 3167: note = a, c, t, g, unknown or other
  misc_difference 3168: note = a, c, t, g, unknown or other
  misc_difference 3169: note = a, c, t, g, unknown or other
  misc_difference 3170: note = a, c, t, g, unknown or other
  misc_difference 3171: note = a, c, t, g, unknown or other
  misc_difference 3172: note = a, c, t, g, unknown or other
  misc_difference 3173: note = a, c, t, g, unknown or other
  misc_difference 3174: note = a, c, t, g, unknown or other
  misc_difference 3175: note = a, c, t, g, unknown or other
  misc_difference 3176: note = a, c, t, g, unknown or other
atacctcatcatataaagcggcgcacttccgaatggctgagttttccacgcccgtccgcagcgagatcgcgacggaggagcgatcgagcgtcccgagggcgggtgccggaggtgagtttacacaccgcagtcaaggggcaattcgggctcgggactggccgggctatggggcaagactcttaaaaaagccatgtttctcggtaaactttacagaaagaaaagggcactgtcactgctacgcgtgcgagctccagaggcgaaaccacctgctatgagttggagacccccggtgcacaaccccaatgggatcgagagaaacctgtgggaggcattctttcgcatgcatgcttcagcttgtggttgtggcgatcttgttggccatcttactgtactggctggtcggtatggtgctcctcctcgtcccccggcccccggcgctcccagaccaccgctgatacgccagctggcccttccggcgccccccgccgatcctcaacaggctaacccacaatggcctggtggggacggtggagaagatggcgctggaggccccgccgctggcggcgccgtcgcagacgccgagtaccaagaagacgagctcaacgccctgttcgacgccgtcgagcaagaagagtaaggaggaggcgatgggggaggcggaggtggagacgggggtacagacgcagactgagactaagacgcagacgcagacgaaagcgaaagatagtactaactcagtggaatcccgccaaagtgcggaggtgtactattaagggagttctgcccatgatcctgtgcggggccgggcgctcggggtttaactacggactgcacagcgacgactacactgtacagaagccccttggccagaacccccacgggggcggcatgagtacagtgacttttagcctacaggtgctctatgaccagtaccagaggtttatgaacaagtggtcgtactccaacgaccagctagacctcgccaggtactttggctgcaccttctggttctacagacacccagaggtggactttgtagctcagtttgacaacgttccccccatgaaaatggacgagaacacagccccaaacactcatccctctttcttactacagaacaaacacaaggttaaaattcccagctttaaaacaaagccttttggtaaaaaaagagttagagttacagtagggccccccaaactgtttgaagataagtggtacagccaacatgacttgtgtaaggtgcccctagtcagttggcggttaaccgcagctgacttcaggtttccgttctgctcaccacaaactgacaacccttgctacaccttccaggtattgcatgaagagtattacccagtaataggcacttctgctttagaaaacggcagtaactacaatagctcagctataacagccttagaaaaattcttatatgaaaaatgcacacactatcaaacatttgccacagacaccagacttaatcctcagcgaccagtgtcatctacaaatgcaaacaaaacatacaccccctcaggctcccaagaaacaatagtgtgggggcagtcagattttaatttatttaaaaagcacacagacagcaactatggctactgcacctactgtcctaccaatgacttagctacaaaaattaaaaagtacagagacaaaagattcgactggctaacaaacatgccagtaacaaacacctgccacataaatgccaccttcgcccgaggcaaaattaaagaatgggagtaccacctagggtggttctcaaacatctttataggcaacctgagacacaacctagcattccgggccgcatacatagacatcacctanacagacaagggagaaggcaacattatctggttccagtacctcactaaacccaccacagagtacatagaagcccaagcaaagtgctccatcacaaacatacccctgtatgctgctttttatggctacgaagactacctccagagaacactaggcccctaccaagatgtagaaaccctaggtataatctgtgttaaatgtccctacacagatccccctctagttcacaagtctacagataaaaagaactggggctacgtgttctacgacgtgcactttggcaacggaaagaccccagagggactgggccaggtgcacccttactggatgcagaggtggagaccctacgtacagtttcagaaagacactatgaacaaaatagccaggacgggaccgttcagctacagagacgagacgccttccatcaccctgaccgccgggtacaagtttcattttaactgggggggcgactctatatttccacagattattaaaaacccctgcccagacagcggggtacgaccttcatccagtagagagcgtcgctcagtacaagtcgttagcccgctcacaatggggccagagtacatattccaccggtgggactggcgacgggggttctttaatcaaaaagctctcaaaagaatgcttgaaaaatcaattaatgatggagagtatccaacaggcccaaaggtccctcgatggtttcccccactcgacaaccaagagcaagaaggcgcctcaggttcagaggagacaaggtcgcagtcctcgcaagaagaagccgctcaagaagccctccaagaagtccaagaggcgtcgctacagcagcacctcctccagcagtaccgagagcagcgacggatcggaaagcaactccaactcgtcatgctgcagctcaccaagacgcagagcaacctgcacataaacccccgtgttcttggccatgcataaataaagtctacatgtttccccccgacaagcccatgcccatacacgggtaccacgggtgggagacggagtaccaggcctgcaaggccttcaacaggccccccagaaactacctttcagacaaacccatctacccttggctccctcgccccgaacccgaaataatagtgagctttaggttcggtttcaaataaacaaggccgcaaataaacaaggccgtgggagtttcactggtcggtgtctacctcttaaggtcactaagcactccgagcgttagcgaggagtgcgacccttccccctggtgccacgccctcggcggccgcgcgctacgcctnnnnnnnnnnnnnnnnnnnnnnnnnnnnnnnnnnnnnnnnnnnnnnnnnnnntgaatcagtaacgaaagtgagtggggccagacttcgccataaggcctttatcttcttgccattggtccgtgtggggagtcgccataggcttcgggctcggttttaggccttccggactacaaaaaccgccattttagtgacgtcacggcggccattttaagtaagcatggcgggcggtgacgtacaagttgaaaggtcaccgcgcttccgtgtttactcaaaatggtggccaactgcttccgggtcaaaggtcggcggccacgtcataagtcacgtgggagggctgcgtcacaaacacggaagtggctgtcccacgtgacttgtcacgtgattgctacgtcacggccgccattttagttcacaaaatggcggacttc

SEQ ID NO: 863 - protein, 121 aa, Alphatorquevirus sp.
MSWRPPVHNPNGIERNLWEAFFRMHASACGCGDLVGHLTVLAGRYGAPPRPPAPGAPRPPLIRQLALPAPPADPQQANPQWPGGDGGEDGAGGPAAGGAVADAEYQEDELNALFDAVEQEE

SEQ ID NO: 864 - protein, 267 aa, Alphatorquevirus sp.
MSWRPPVHNPNGIERNLWEAFFRMHASACGCGDLVGHLTVLAGRYGAPPRPPAPGAPRPPLIRQLALPAPPADPQQANPQWPGGDGGEDGAGGPAAGGAVADAEYQEDELNALFDAVEQEELLKTPAQTAGYDLHPVESVAQYKSLARSQWGQSTYSTGGTGDGGSLIKKLSKECLKNQLMMESIQQAQRSLDGFPHSTTKSKKAPQVQRRQGRSPRKKKPLKKPSKKSKRRRYSSTSSSSTESSDGSESNSNSSCCSSPRRRATCT

SEQ ID NO: 865 - protein, 273 aa, Alphatorquevirus sp.
MSWRPPVHNPNGIERNLWEAFFRMHASACGCGDLVGHLTVLAGRYGAPPRPPAPGAPRPPLIRQLALPAPPADPQQANPQWPGGDGGEDGAGGPAAGGAVADAEYQEDELNALFDAVEQEEPKGPSMVSPTRQPRARRRLRFRGDKVAVLARRSRSRSPPRSPRGVATAAPPPAVPRAATDRKATPTRHAAAHQDAEQPAHKPPCSWPCINKVYMFPPDKPMPIHGYHGWETEYQACKAFNRPPRNYLSDKPIYPWLPRPEPEIIVSFRFGFK

SEQ ID NO: 866 - protein, 196 aa, Alphatorquevirus sp.
MSWRPPVHNPNGIERNLWEAFFRMHASACGCGDLVGHLTVLAGRPKGPSMVSPTRQPRARRRLRFRGDKVAVLARRSRSRSPPRSPRGVATAAPPPAVPRAATDRKATPTRHAAAHQDAEQPAHKPPCSWPCINKVYMFPPDKPMPIHGYHGWETEYQACKAFNRPPRNYLSDKPIYPWLPRPEPEIIVSFRFGFK

SEQ ID NO: 867 - protein, 760 aa, Alphatorquevirus sp.
  VARIANT 444: note = Any amino acid
MAWWGRWRRWRWRPRRWRRRRRRRVPRRRAQRPVRRRRARRVRRRRWGRRRWRRGYRRRLRLRRRRRRKRKIVLTQWNPAKVRRCTIKGVLPMILCGAGRSGFNYGLHSDDYTVQKPLGQNPHGGGMSTVTFSLQVLYDQYQRFMNKWSYSNDQLDLARYFGCTFWFYRHPEVDFVAQFDNVPPMKMDENTAPNTHPSFLLQNKHKVKIPSFKTKPFGKKRVRVTVGPPKLFEDKWYSQHDLCKVPLVSWRLTAADFRFPFCSPQTDNPCYTFQVLHEEYYPVIGTSALENGSNYNSSAITALEKFLYEKCTHYQTFATDTRLNPQRPVSSTNANKTYTPSGSQETIVWGQSDFNLFKKHTDSNYGYCTYCPTNDLATKIKKYRDKRFDWLTNMPVTNTCHINATFARGKIKEWEYHLGWFSNIFIGNLRHNLAFRAAYIDITXTDKGEGNIIWFQYLTKPTTEYIEAQAKCSITNIPLYAAFYGYEDYLQRTLGPYQDVETLGIICVKCPYTDPPLVHKSTDKKNWGYVFYDVHFGNGKTPEGLGQVHPYWMQRWRPYVQFQKDTMNKIARTGPFSYRDETPSITLTAGYKFHFNWGGDSIFPQIIKNPCPDSGVRPSSSRERRSVQVVSPLTMGPEYIFHRWDWRRGFFNQKALKRMLEKSINDGEYPTGPKVPRWFPPLDNQEQEGASGSEETRSQSSQEEAAQEALQEVQEASLQQHLLQQYREQRRIGKQLQLVMLQLTKTQSNLHINPRVLGHA

SEQ ID NO: 868 - protein, 196 aa, Alphatorquevirus sp.
MAWWGRWRRWRWRPRRWRRRRRRRVPRRRAQRPVRRRRARRIIKNPCPDSGVRPSSSRERRSVQVVSPLTMGPEYIFHRWDWRRGFFNQKALKRMLEKSINDGEYPTGPKVPRWFPPLDNQEQEGASGSEETRSQSSQEEAAQEALQEVQEASLQQHLLQQYREQRRIGKQLQLVMLQLTKTQSNLHINPRVLGHA

SEQ ID NO: 869 - protein, 121 aa, Alphatorquevirus sp.
MAWWGRWRRWRWRPRRWRRRRRRRVPRRRAQRPVRRRRARRAQRSLDGFPHSTTKSKKAPQVQRRQGRSPRKKKPLKKPSKKSKRRRYSSTSSSSTESSDGSESNSNSSCCSSPRRRATCT

SEQ ID NO: 870 - DNA, 3896 bp, Alphatorquevirus sp.
  source 1..3896: mol_type = genomic DNA
  misc_difference 1276: note = a, c, t, g, unknown or other
  misc_difference 3198: note = a, c, t, g, unknown or other
  misc_difference 3199: note = a, c, t, g, unknown or other
  misc_difference 3200: note = a, c, t, g, unknown or other
  misc_difference 3201: note = a, c, t, g, unknown or other
  misc_difference 3202: note = a, c, t, g, unknown or other
  misc_difference 3203: note = a, c, t, g, unknown or other
  misc_difference 3204: note = a, c, t, g, unknown or other
  misc_difference 3205: note = a, c, t, g, unknown or other
  misc_difference 3206: note = a, c, t, g, unknown or other
  misc_difference 3207: note = a, c, t, g, unknown or other
  misc_difference 3208: note = a, c, t, g, unknown or other
  misc_difference 3209: note = a, c, t, g, unknown or other
  misc_difference 3210: note = a, c, t, g, unknown or other
  misc_difference 3211: note = a, c, t, g, unknown or other
  misc_difference 3212: note = a, c, t, g, unknown or other
  misc_difference 3213: note = a, c, t, g, unknown or other
  misc_difference 3214: note = a, c, t, g, unknown or other
  misc_difference 3215: note = a, c, t, g, unknown or other
  misc_difference 3216: note = a, c, t, g, unknown or other
  misc_difference 3217: note = a, c, t, g, unknown or other
  misc_difference 3218: note = a, c, t, g, unknown or other
  misc_difference 3219: note = a, c, t, g, unknown or other
  misc_difference 3220: note = a, c, t, g, unknown or other
  misc_difference 3221: note = a, c, t, g, unknown or other
  misc_difference 3222: note = a, c, t, g, unknown or other
  misc_difference 3223: note = a, c, t, g, unknown or other
  misc_difference 3224: note = a, c, t, g, unknown or other
  misc_difference 3225: note = a, c, t, g, unknown or other
  misc_difference 3226: note = a, c, t, g, unknown or other
  misc_difference 3227: note = a, c, t, g, unknown or other
  misc_difference 3228: note = a, c, t, g, unknown or other
  misc_difference 3229: note = a, c, t, g, unknown or other
  misc_difference 3230: note = a, c, t, g, unknown or other
  misc_difference 3231: note = a, c, t, g, unknown or other
  misc_difference 3232: note = a, c, t, g, unknown or other
  misc_difference 3233: note = a, c, t, g, unknown or other
  misc_difference 3234: note = a, c, t, g, unknown or other
  misc_difference 3235: note = a, c, t, g, unknown or other
  misc_difference 3236: note = a, c, t, g, unknown or other
  misc_difference 3237: note = a, c, t, g, unknown or other
  misc_difference 3238: note = a, c, t, g, unknown or other
  misc_difference 3239: note = a, c, t, g, unknown or other
  misc_difference 3240: note = a, c, t, g, unknown or other
  misc_difference 3241: note = a, c, t, g, unknown or other
  misc_difference 3242: note = a, c, t, g, unknown or other
  misc_difference 3243: note = a, c, t, g, unknown or other
  misc_difference 3244: note = a, c, t, g, unknown or other
  misc_difference 3245: note = a, c, t, g, unknown or other
  misc_difference 3246: note = a, c, t, g, unknown or other
  misc_difference 3247: note = a, c, t, g, unknown or other
  misc_difference 3248: note = a, c, t, g, unknown or other
  misc_difference 3249: note = a, c, t, g, unknown or other
  misc_difference 3250: note = a, c, t, g, unknown or other
  misc_difference 3251: note = a, c, t, g, unknown or other
  misc_difference 3252: note = a, c, t, g, unknown or other
  misc_difference 3253: note = a, c, t, g, unknown or other
  misc_difference 3254: note = a, c, t, g, unknown or other
  misc_difference 3255: note = a, c, t, g, unknown or other
  misc_difference 3256: note = a, c, t, g, unknown or other
  misc_difference 3257: note = a, c, t, g, unknown or other
  misc_difference 3258: note = a, c, t, g, unknown or other
  misc_difference 3259: note = a, c, t, g, unknown or other
  misc_difference 3260: note = a, c, t, g, unknown or other
  misc_difference 3261: note = a, c, t, g, unknown or other
  misc_difference 3262: note = a, c, t, g, unknown or other
  misc_difference 3263: note = a, c, t, g, unknown or other
  misc_difference 3264: note = a, c, t, g, unknown or other
  misc_difference 3265: note = a, c, t, g, unknown or other
  misc_difference 3266: note = a, c, t, g, unknown or other
  misc_difference 3267: note = a, c, t, g, unknown or other
  misc_difference 3268: note = a, c, t, g, unknown or other
  misc_difference 3269: note = a, c, t, g, unknown or other
  misc_difference 3270: note = a, c, t, g, unknown or other
  misc_difference 3271: note = a, c, t, g, unknown or other
  misc_difference 3272: note = a, c, t, g, unknown or other
  misc_difference 3273: note = a, c, t, g, unknown or other
  misc_difference 3274: note = a, c, t, g, unknown or other
  misc_difference 3275: note = a, c, t, g, unknown or other
  misc_difference 3276: note = a, c, t, g, unknown or other
  misc_difference 3277: note = a, c, t, g, unknown or other
  misc_difference 3278: note = a, c, t, g, unknown or other
  misc_difference 3279: note = a, c, t, g, unknown or other
  misc_difference 3280: note = a, c, t, g, unknown or other
  misc_difference 3281: note = a, c, t, g, unknown or other
  misc_difference 3282: note = a, c, t, g, unknown or other
  misc_difference 3283: note = a, c, t, g, unknown or other
  misc_difference 3284: note = a, c, t, g, unknown or other
  misc_difference 3285: note = a, c, t, g, unknown or other
  misc_difference 3286: note = a, c, t, g, unknown or other
  misc_difference 3287: note = a, c, t, g, unknown or other
  misc_difference 3288: note = a, c, t, g, unknown or other
  misc_difference 3289: note = a, c, t, g, unknown or other
  misc_difference 3290: note = a, c, t, g, unknown or other
  misc_difference 3291: note = a, c, t, g, unknown or other
  misc_difference 3292: note = a, c, t, g, unknown or other
  misc_difference 3293: note = a, c, t, g, unknown or other
  misc_difference 3294: note = a, c, t, g, unknown or other
  misc_difference 3295: note = a, c, t, g, unknown or other
  misc_difference 3296: note = a, c, t, g, unknown or other
  misc_difference 3297: note = a, c, t, g, unknown or other
  misc_difference 3298: note = a, c, t, g, unknown or other
  misc_difference 3299: note = a, c, t, g, unknown or other
  misc_difference 3300: note = a, c, t, g, unknown or other
  misc_difference 3301: note = a, c, t, g, unknown or other
  misc_difference 3302: note = a, c, t, g, unknown or other
  misc_difference 3303: note = a, c, t, g, unknown or other
  misc_difference 3304: note = a, c, t, g, unknown or other
  misc_difference 3305: note = a, c, t, g, unknown or other
  misc_difference 3306: note = a, c, t, g, unknown or other
  misc_difference 3307: note = a, c, t, g, unknown or other
  misc_difference 3308: note = a, c, t, g, unknown or other
  misc_difference 3309: note = a, c, t, g, unknown or other
  misc_difference 3310: note = a, c, t, g, unknown or other
  misc_difference 3311: note = a, c, t, g, unknown or other
  misc_difference 3312: note = a, c, t, g, unknown or other
  misc_difference 3313: note = a, c, t, g, unknown or other
  misc_difference 3314: note = a, c, t, g, unknown or other
  misc_difference 3315: note = a, c, t, g, unknown or other
  misc_difference 3316: note = a, c, t, g, unknown or other
  misc_difference 3317: note = a, c, t, g, unknown or other
  misc_difference 3318: note = a, c, t, g, unknown or other
  misc_difference 3319: note = a, c, t, g, unknown or other
  misc_difference 3320: note = a, c, t, g, unknown or other
  misc_difference 3321: note = a, c, t, g, unknown or other
  misc_difference 3322: note = a, c, t, g, unknown or other
  misc_difference 3323: note = a, c, t, g, unknown or other
  misc_difference 3324: note = a, c, t, g, unknown or other
  misc_difference 3325: note = a, c, t, g, unknown or other
  misc_difference 3326: note = a, c, t, g, unknown or other
  misc_difference 3327: note = a, c, t, g, unknown or other
  misc_difference 3328: note = a, c, t, g, unknown or other
  misc_difference 3329: note = a, c, t, g, unknown or other
  misc_difference 3330: note = a, c, t, g, unknown or other
  misc_difference 3331: note = a, c, t, g, unknown or other
  misc_difference 3332: note = a, c, t, g, unknown or other
  misc_difference 3333: note = a, c, t, g, unknown or other
  misc_difference 3334: note = a, c, t, g, unknown or other
  misc_difference 3335: note = a, c, t, g, unknown or other
  misc_difference 3336: note = a, c, t, g, unknown or other
  misc_difference 3337: note = a, c, t, g, unknown or other
  misc_difference 3338: note = a, c, t, g, unknown or other
  misc_difference 3339: note = a, c, t, g, unknown or other
  misc_difference 3340: note = a, c, t, g, unknown or other
  misc_difference 3341: note = a, c, t, g, unknown or other
  misc_difference 3342: note = a, c, t, g, unknown or other
  misc_difference 3343: note = a, c, t, g, unknown or other
  misc_difference 3344: note = a, c, t, g, unknown or other
  misc_difference 3345: note = a, c, t, g, unknown or other
  misc_difference 3346: note = a, c, t, g, unknown or other
  misc_difference 3347: note = a, c, t, g, unknown or other
  misc_difference 3348: note = a, c, t, g, unknown or other
  misc_difference 3349: note = a, c, t, g, unknown or other
  misc_difference 3350: note = a, c, t, g, unknown or other
  misc_difference 3351: note = a, c, t, g, unknown or other
  misc_difference 3352: note = a, c, t, g, unknown or other
  misc_difference 3353: note = a, c, t, g, unknown or other
  misc_difference 3354: note = a, c, t, g, unknown or other
  misc_difference 3355: note = a, c, t, g, unknown or other
  misc_difference 3356: note = a, c, t, g, unknown or other
  misc_difference 3357: note = a, c, t, g, unknown or other
  misc_difference 3358: note = a, c, t, g, unknown or other
  misc_difference 3359: note = a, c, t, g, unknown or other
  misc_difference 3360: note = a, c, t, g, unknown or other
  misc_difference 3361: note = a, c, t, g, unknown or other
  misc_difference 3362: note = a, c, t, g, unknown or other
  misc_difference 3363: note = a, c, t, g, unknown or other
  misc_difference 3364: note = a, c, t, g, unknown or other
  misc_difference 3365: note = a, c, t, g, unknown or other
  misc_difference 3366: note = a, c, t, g, unknown or other
  misc_difference 3367: note = a, c, t, g, unknown or other
  misc_difference 3368: note = a, c, t, g, unknown or other
  misc_difference 3369: note = a, c, t, g, unknown or other
  misc_difference 3370: note = a, c, t, g, unknown or other
  misc_difference 3371: note = a, c, t, g, unknown or other
  misc_difference 3372: note = a, c, t, g, unknown or other
  misc_difference 3373: note = a, c, t, g, unknown or other
  misc_difference 3374: note = a, c, t, g, unknown or other
  misc_difference 3375: note = a, c, t, g, unknown or other
  misc_difference 3376: note = a, c, t, g, unknown or other
  misc_difference 3377: note = a, c, t, g, unknown or other
  misc_difference 3378: note = a, c, t, g, unknown or other
  misc_difference 3379: note = a, c, t, g, unknown or other
  misc_difference 3380: note = a, c, t, g, unknown or other
  misc_difference 3381: note = a, c, t, g, unknown or other
  misc_difference 3382: note = a, c, t, g, unknown or other
  misc_difference 3383: note = a, c, t, g, unknown or other
  misc_difference 3384: note = a, c, t, g, unknown or other
  misc_difference 3385: note = a, c, t, g, unknown or other
  misc_difference 3386: note = a, c, t, g, unknown or other
  misc_difference 3387: note = a, c, t, g, unknown or other
  misc_difference 3388: note = a, c, t, g, unknown or other
  misc_difference 3389: note = a, c, t, g, unknown or other
  misc_difference 3390: note = a, c, t, g, unknown or other
  misc_difference 3391: note = a, c, t, g, unknown or other
  misc_difference 3392: note = a, c, t, g, unknown or other
  misc_difference 3393: note = a, c, t, g, unknown or other
  misc_difference 3394: note = a, c, t, g, unknown or other
  misc_difference 3395: note = a, c, t, g, unknown or other
  misc_difference 3396: note = a, c, t, g, unknown or other
  misc_difference 3397: note = a, c, t, g, unknown or other
  misc_difference 3398: note = a, c, t, g, unknown or other
  misc_difference 3399: note = a, c, t, g, unknown or other
  misc_difference 3400: note = a, c, t, g, unknown or other
  misc_difference 3401: note = a, c, t, g, unknown or other
  misc_difference 3402: note = a, c, t, g, unknown or other
  misc_difference 3403: note = a, c, t, g, unknown or other
  misc_difference 3404: note = a, c, t, g, unknown or other
  misc_difference 3405: note = a, c, t, g, unknown or other
  misc_difference 3406: note = a, c, t, g, unknown or other
  misc_difference 3407: note = a, c, t, g, unknown or other
  misc_difference 3408: note = a, c, t, g, unknown or other
  misc_difference 3409: note = a, c, t, g, unknown or other
  misc_difference 3410: note = a, c, t, g, unknown or other
  misc_difference 3411: note = a, c, t, g, unknown or other
  misc_difference 3412: note = a, c, t, g, unknown or other
  misc_difference 3413: note = a, c, t, g, unknown or other
  misc_difference 3414: note = a, c, t, g, unknown or other
  misc_difference 3415: note = a, c, t, g, unknown or other
  misc_difference 3416: note = a, c, t, g, unknown or other
  misc_difference 3417: note = a, c, t, g, unknown or other
  misc_difference 3418: note = a, c, t, g, unknown or other
  misc_difference 3419: note = a, c, t, g, unknown or other
  misc_difference 3420: note = a, c, t, g, unknown or other
  misc_difference 3421: note = a, c, t, g, unknown or other
  misc_difference 3422: note = a, c, t, g, unknown or other
  misc_difference 3423: note = a, c, t, g, unknown or other
  misc_difference 3424: note = a, c, t, g, unknown or other
  misc_difference 3425: note = a, c, t, g, unknown or other
  misc_difference 3426: note = a, c, t, g, unknown or other
  misc_difference 3427: note = a, c, t, g, unknown or other
  misc_difference 3428: note = a, c, t, g, unknown or other
  misc_difference 3429: note = a, c, t, g, unknown or other
  misc_difference 3430: note = a, c, t, g, unknown or other
  misc_difference 3431: note = a, c, t, g, unknown or other
  misc_difference 3432: note = a, c, t, g, unknown or other
  misc_difference 3433: note = a, c, t, g, unknown or other
  misc_difference 3434: note = a, c, t, g, unknown or other
  misc_difference 3435: note = a, c, t, g, unknown or other
  misc_difference 3436: note = a, c, t, g, unknown or other
  misc_difference 3437: note = a, c, t, g, unknown or other
  misc_difference 3438: note = a, c, t, g, unknown or other
  misc_difference 3439: note = a, c, t, g, unknown or other
  misc_difference 3440: note = a, c, t, g, unknown or other
  misc_difference 3441: note = a, c, t, g, unknown or other
  misc_difference 3442: note = a, c, t, g, unknown or other
  misc_difference 3443: note = a, c, t, g, unknown or other
  misc_difference 3444: note = a, c, t, g, unknown or other
  misc_difference 3445: note = a, c, t, g, unknown or other
  misc_difference 3446: note = a, c, t, g, unknown or other
  misc_difference 3447: note = a, c, t, g, unknown or other
  misc_difference 3448: note = a, c, t, g, unknown or other
  misc_difference 3449: note = a, c, t, g, unknown or other
  misc_difference 3450: note = a, c, t, g, unknown or other
  misc_difference 3451: note = a, c, t, g, unknown or other
  misc_difference 3452: note = a, c, t, g, unknown or other
  misc_difference 3453: note = a, c, t, g, unknown or other
  misc_difference 3454: note = a, c, t, g, unknown or other
  misc_difference 3455: note = a, c, t, g, unknown or other
  misc_difference 3456: note = a, c, t, g, unknown or other
  misc_difference 3457: note = a, c, t, g, unknown or other
  misc_difference 3458: note = a, c, t, g, unknown or other
  misc_difference 3459: note = a, c, t, g, unknown or other
  misc_difference 3460: note = a, c, t, g, unknown or other
  misc_difference 3461: note = a, c, t, g, unknown or other
  misc_difference 3462: note = a, c, t, g, unknown or other
  misc_difference 3463: note = a, c, t, g, unknown or other
  misc_difference 3464: note = a, c, t, g, unknown or other
  misc_difference 3465: note = a, c, t, g, unknown or other
  misc_difference 3466: note = a, c, t, g, unknown or other
  misc_difference 3467: note = a, c, t, g, unknown or other
  misc_difference 3468: note = a, c, t, g, unknown or other
  misc_difference 3469: note = a, c, t, g, unknown or other
  misc_difference 3470: note = a, c, t, g, unknown or other
  misc_difference 3471: note = a, c, t, g, unknown or other
  misc_difference 3472: note = a, c, t, g, unknown or other
  misc_difference 3473: note = a, c, t, g, unknown or other
  misc_difference 3474: note = a, c, t, g, unknown or other
  misc_difference 3475: note = a, c, t, g, unknown or other
  misc_difference 3476: note = a, c, t, g, unknown or other
  misc_difference 3477: note = a, c, t, g, unknown or other
  misc_difference 3478: note = a, c, t, g, unknown or other
  misc_difference 3479: note = a, c, t, g, unknown or other
  misc_difference 3480: note = a, c, t, g, unknown or other
  misc_difference 3481: note = a, c, t, g, unknown or other
  misc_difference 3482: note = a, c, t, g, unknown or other
  misc_difference 3483: note = a, c, t, g, unknown or other
  misc_difference 3484: note = a, c, t, g, unknown or other
  misc_difference 3485: note = a, c, t, g, unknown or other
  misc_difference 3486: note = a, c, t, g, unknown or other
  misc_difference 3487: note = a, c, t, g, unknown or other
  misc_difference 3488: note = a, c, t, g, unknown or other
  misc_difference 3489: note = a, c, t, g, unknown or other
  misc_difference 3490: note = a, c, t, g, unknown or other
  misc_difference 3491: note = a, c, t, g, unknown or other
  misc_difference 3492: note = a, c, t, g, unknown or other
  misc_difference 3493: note = a, c, t, g, unknown or other
  misc_difference 3494: note = a, c, t, g, unknown or other
  misc_difference 3495: note = a, c, t, g, unknown or other
  misc_difference 3496: note = a, c, t, g, unknown or other
  misc_difference 3497: note = a, c, t, g, unknown or other
  misc_difference 3498: note = a, c, t, g, unknown or other
  misc_difference 3499: note = a, c, t, g, unknown or other
  misc_difference 3500: note = a, c, t, g, unknown or other
  misc_difference 3501: note = a, c, t, g, unknown or other
  misc_difference 3502: note = a, c, t, g, unknown or other
  misc_difference 3503: note = a, c, t, g, unknown or other
  misc_difference 3504: note = a, c, t, g, unknown or other
  misc_difference 3505: note = a, c, t, g, unknown or other
  misc_difference 3506: note = a, c, t, g, unknown or other
  misc_difference 3507: note = a, c, t, g, unknown or other
  misc_difference 3508: note = a, c, t, g, unknown or other
  misc_difference 3509: note = a, c, t, g, unknown or other
  misc_difference 3510: note = a, c, t, g, unknown or other
  misc_difference 3511: note = a, c, t, g, unknown or other
  misc_difference 3512: note = a, c, t, g, unknown or other
  misc_difference 3513: note = a, c, t, g, unknown or other
  misc_difference 3514: note = a, c, t, g, unknown or other
  misc_difference 3515: note = a, c, t, g, unknown or other
  misc_difference 3516: note = a, c, t, g, unknown or other
  misc_difference 3517: note = a, c, t, g, unknown or other
  misc_difference 3518: note = a, c, t, g, unknown or other
  misc_difference 3519: note = a, c, t, g, unknown or other
  misc_difference 3520: note = a, c, t, g, unknown or other
  misc_difference 3521: note = a, c, t, g, unknown or other
  misc_difference 3522: note = a, c, t, g, unknown or other
  misc_difference 3523: note = a, c, t, g, unknown or other
  misc_difference 3524: note = a, c, t, g, unknown or other
  misc_difference 3525: note = a, c, t, g, unknown or other
  misc_difference 3526: note = a, c, t, g, unknown or other
  misc_difference 3527: note = a, c, t, g, unknown or other
  misc_difference 3528: note = a, c, t, g, unknown or other
  misc_difference 3529: note = a, c, t, g, unknown or other
  misc_difference 3530: note = a, c, t, g, unknown or other
  misc_difference 3531: note = a, c, t, g, unknown or other
  misc_difference 3532: note = a, c, t, g, unknown or other
  misc_difference 3533: note = a, c, t, g, unknown or other
  misc_difference 3534: note = a, c, t, g, unknown or other
  misc_difference 3535: note = a, c, t, g, unknown or other
  misc_difference 3536: note = a, c, t, g, unknown or other
  misc_difference 3537: note = a, c, t, g, unknown or other
  misc_difference 3538: note = a, c, t, g, unknown or other
  misc_difference 3539: note = a, c, t, g, unknown or other
  misc_difference 3540: note = a, c, t, g, unknown or other
  misc_difference 3541: note = a, c, t, g, unknown or other
  misc_difference 3542: note = a, c, t, g, unknown or other
  misc_difference 3543: note = a, c, t, g, unknown or other
  misc_difference 3544: note = a, c, t, g, unknown or other
  misc_difference 3545: note = a, c, t, g, unknown or other
  misc_difference 3546: note = a, c, t, g, unknown or other
  misc_difference 3547: note = a, c, t, g, unknown or other
  misc_difference 3548: note = a, c, t, g, unknown or other
  misc_difference 3549: note = a, c, t, g, unknown or other
  misc_difference 3550: note = a, c, t, g, unknown or other
  misc_difference 3551: note = a, c, t, g, unknown or other
  misc_difference 3552: note = a, c, t, g, unknown or other
  misc_difference 3553: note = a, c, t, g, unknown or other
  misc_difference 3554: note = a, c, t, g, unknown or other
  misc_difference 3555: note = a, c, t, g, unknown or other
  misc_difference 3556: note = a, c, t, g, unknown or other
  misc_difference 3557: note = a, c, t, g, unknown or other
  misc_difference 3558: note = a, c, t, g, unknown or other
  misc_difference 3559: note = a, c, t, g, unknown or other
  misc_difference 3560: note = a, c, t, g, unknown or other
  misc_difference 3561: note = a, c, t, g, unknown or other
  misc_difference 3562: note = a, c, t, g, unknown or other
  misc_difference 3563: note = a, c, t, g, unknown or other
  misc_difference 3564: note = a, c, t, g, unknown or other
  misc_difference 3565: note = a, c, t, g, unknown or other
  misc_difference 3566: note = a, c, t, g, unknown or other
  misc_difference 3567: note = a, c, t, g, unknown or other
  misc_difference 3568: note = a, c, t, g, unknown or other
  misc_difference 3569: note = a, c, t, g, unknown or other
  misc_difference 3570: note = a, c, t, g, unknown or other
  misc_difference 3571: note = a, c, t, g, unknown or other
  misc_difference 3572: note = a, c, t, g, unknown or other
  misc_difference 3573: note = a, c, t, g, unknown or other
  misc_difference 3574: note = a, c, t, g, unknown or other
  misc_difference 3575: note = a, c, t, g, unknown or other
  misc_difference 3576: note = a, c, t, g, unknown or other
  misc_difference 3577: note = a, c, t, g, unknown or other
  misc_difference 3578: note = a, c, t, g, unknown or other
  misc_difference 3579: note = a, c, t, g, unknown or other
  misc_difference 3580: note = a, c, t, g, unknown or other
  misc_difference 3581: note = a, c, t, g, unknown or other
  misc_difference 3582: note = a, c, t, g, unknown or other
  misc_difference 3583: note = a, c, t, g, unknown or other
  misc_difference 3584: note = a, c, t, g, unknown or other
  misc_difference 3585: note = a, c, t, g, unknown or other
  misc_difference 3586: note = a, c, t, g, unknown or other
  misc_difference 3587: note = a, c, t, g, unknown or other
  misc_difference 3588: note = a, c, t, g, unknown or other
  misc_difference 3589: note = a, c, t, g, unknown or other
  misc_difference 3590: note = a, c, t, g, unknown or other
  misc_difference 3591: note = a, c, t, g, unknown or other
  misc_difference 3592: note = a, c, t, g, unknown or other
  misc_difference 3593: note = a, c, t, g, unknown or other
  misc_difference 3594: note = a, c, t, g, unknown or other
  misc_difference 3595: note = a, c, t, g, unknown or other
  misc_difference 3596: note = a, c, t, g, unknown or other
  misc_difference 3597: note = a, c, t, g, unknown or other
  misc_difference 3598: note = a, c, t, g, unknown or other
  misc_difference 3599: note = a, c, t, g, unknown or other
  misc_difference 3600: note = a, c, t, g, unknown or other
  misc_difference 3601: note = a, c, t, g, unknown or other
  misc_difference 3602: note = a, c, t, g, unknown or other
  misc_difference 3603: note = a, c, t, g, unknown or other
  misc_difference 3604: note = a, c, t, g, unknown or other
  misc_difference 3605: note = a, c, t, g, unknown or other
  misc_difference 3606: note = a, c, t, g, unknown or other
  misc_difference 3607: note = a, c, t, g, unknown or other
  misc_difference 3608: note = a, c, t, g, unknown or other
  misc_difference 3609: note = a, c, t, g, unknown or other
  misc_difference 3610: note = a, c, t, g, unknown or other
  misc_difference 3611: note = a, c, t, g, unknown or other
  misc_difference 3612: note = a, c, t, g, unknown or other
  misc_difference 3613: note = a, c, t, g, unknown or other
  misc_difference 3614: note = a, c, t, g, unknown or other
  misc_difference 3615: note = a, c, t, g, unknown or other
  misc_difference 3616: note = a, c, t, g, unknown or other
  misc_difference 3617: note = a, c, t, g, unknown or other
  misc_difference 3618: note = a, c, t, g, unknown or other
  misc_difference 3619: note = a, c, t, g, unknown or other
  misc_difference 3620: note = a, c, t, g, unknown or other
  misc_difference 3621: note = a, c, t, g, unknown or other
  misc_difference 3622: note = a, c, t, g, unknown or other
  misc_difference 3623: note = a, c, t, g, unknown or other
  misc_difference 3624: note = a, c, t, g, unknown or other
  misc_difference 3625: note = a, c, t, g, unknown or other
  misc_difference 3626: note = a, c, t, g, unknown or other
  misc_difference 3627: note = a, c, t, g, unknown or other
  misc_difference 3628: note = a, c, t, g, unknown or other
  misc_difference 3629: note = a, c, t, g, unknown or other
  misc_difference 3630: note = a, c, t, g, unknown or other
  misc_difference 3631: note = a, c, t, g, unknown or other
  misc_difference 3632: note = a, c, t, g, unknown or other
  misc_difference 3633: note = a, c, t, g, unknown or other
  misc_difference 3634: note = a, c, t, g, unknown or other
  misc_difference 3635: note = a, c, t, g, unknown or other
  misc_difference 3636: note = a, c, t, g, unknown or other
  misc_difference 3637: note = a, c, t, g, unknown or other
  misc_difference 3638: note = a, c, t, g, unknown or other
  misc_difference 3639: note = a, c, t, g, unknown or other
  misc_difference 3640: note = a, c, t, g, unknown or other
  misc_difference 3641: note = a, c, t, g, unknown or other
  misc_difference 3642: note = a, c, t, g, unknown or other
  misc_difference 3643: note = a, c, t, g, unknown or other
  misc_difference 3644: note = a, c, t, g, unknown or other
  misc_difference 3645: note = a, c, t, g, unknown or other
  misc_difference 3646: note = a, c, t, g, unknown or other
  misc_difference 3647: note = a, c, t, g, unknown or other
  misc_difference 3648: note = a, c, t, g, unknown or other
  misc_difference 3649: note = a, c, t, g, unknown or other
  misc_difference 3650: note = a, c, t, g, unknown or other
  misc_difference 3651: note = a, c, t, g, unknown or other
  misc_difference 3652: note = a, c, t, g, unknown or other
  misc_difference 3653: note = a, c, t, g, unknown or other
  misc_difference 3654: note = a, c, t, g, unknown or other
  misc_difference 3655: note = a, c, t, g, unknown or other
taaacttcctcttttaataggaaaccacaaaatttgcattgccgaccacaaacgcatatgcaaatttacttccccaaaaactcaaccacaaaatttgcattgccgcccacaaacgtctactttaaccacatcctctaacatgttagaaactccacccaactacttcattagtatacagcatcacaagggaggagccaaacaactatataaccaagtgtacttccgaatggctgagtttatgccgccagacggagacgggatcgcgacggaggagcgatcgagcgtcccgagggcgggtgccggaggtgagtttacacaccgcagtcaaggggcaattcgggctcgggactggccgggctatgggcaaggctcttaaaaaagccatgtttctcggtcgaccttacagaaagaaaagggcactgtcactgctacgcgtgcgagctccagaggcgaaaccacctgctatgagctggaggcccccggtgcacaaccctaatgggatccagagaaacctgtgggaggcattctttcgcatgcatgctgcagcttgtggttgtggcgatcttgttggccatattactgtactggctggtcggtatggtgctcctcctcgtcccccggcccccggggctcccagaccaccgctgatacgccagctggcccttccggcgccccccgccgatcctcaacaggctaacccacaatggcctggtggggacggtggagaagatggcgctggaggccccgccgctggcggcgccgtcgcagacgccgagtaccaagaagacgagctcaacgccctgttcgacgccgtcgagcaagaagagtaaggaggaggcgatgggggaggcggaggtggagacgggggtacagacgcagactaagactgagacgcagacgcagacgaaagaaaataagactgactcagtggaacccagccaaagtcaggagatgtactattaagggggtgctacccatgatcttatgcggcgccggccgctcggggtttaactatggactgcacagcgacgactacacggtgcagaaacccctggggcagaacccccacgggggcggcatgagcacagtaacttttagcctacaagtactatttgaccagtaccagaggtttatgaaccggtggtcgtactccaacgaccagctagacctcgccaggtactttggctgcaccttctacttttacagacaccctgaaattgactttgtagctcagtatgacaatgtacccccaatgaaaatggacgagaacacggcncctaacactcacccctcttttctactacaaaacaaacgcaaaattaaaatccccagctttaaaaccaagccatttggcagaaaaagagtaaaagtaacagtggggccccccaaactgtttgaagataaatggtacagccagcatgacttgtgtaaggtgcccctagtcagttggcggttaaccgcatgtgacttcaggtttccgttctgctcaccactaactgacaacccttgctacaccttccaggtattgcatgaaaactattacccagtcataggcacttcctctttagaaaacggtacaaactacaataacactgctataactacccttgagacatggctatatggaaaatgcacacactatcaaacatttgccacagacaccagacttaatccacagagacctgtatcttcaagtaatgcaaatgaaacttatactcctagtggttctaaagaatcaataatatggggacagtctgactgggcaaactttaaaaagaacacagacagcaactatggctactgttcctactgcccctcaaatggcactaacggaacagtagataaaattaaaaaatacagagaccaaagatttagatggcttacagaaatgccagtacctaacacctgtcacatacatgccaccttcgcccgaggcactattaaatactgggagtaccacctaggctggtactcaaacatatttattggcaacctcagacacaacttagccttcagaccagcctacatagacattacctacaatcccatcactgacaaaggagagggcaacattatctggttccagtacctcactaagcccaccacagaatacatagaaacccaggcaaaatgcaccattactaacattcccctttatgctgctttctatggctacgaagactacctccagagaacactaggcccctaccaagatgtagaaaccctaggcataatctgtgttaaatgtccctacacagatccccctctagttcacaaagacaaaagtaaaaccaactggggctacgtattctacgacgcccactttggcaacggaaagaccccagagggactaggccaagtacacccttactggatgcagagatggagaccctatgtacagtttcaaaaagacaccatgcacaaaatatccagaacgggacccttcagctacagagacgacacgccttccatcaccctcactgccgaatacaagtttcgttttaactgggggggcgactctatatttccacagattattaaaaacccctgcccagacaccggggttcgaccttcaaccggtagagaccgtcgctcagtacaagtcgttagcccgctcacaatgggaccccagtttatattccactcatgggactggagacgggggttctttaatcaaaaaactctcaaaagaatgcttgaaaaaccagttaatgatggagaatatccaacaggcccaaaggtgcctcgatggtttcccccactcgacaaccaagagcaagaaggcgtctcagatacagagacgacaacctcgcagtcctcgcaagaagaagccgctcaagaagccctccaagaagtccaagaggcgtcgctacagcagcacctcctccagcagtaccgagagcagcgaagaatcggaaagcaactccaactcgtcatgctccaactcaccaagacgcagagcaacctgcacataaatccccgtgtccttggccatgcataaataaagtgtacatgtttccccccgaaaagccaatgcccatacacggctaccacgggtgggagacagagtatcaggcctgcaaggcctttgacaggccccctagaaactacctatcagacaaacccatctacccctggcttccccgctcccaaccagaatttaaagtgagttttaagcttggctgtcaataaacaagnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngtttacacaaaatggtggccaagtccttccgggtgaaaggtcggcgcctacgtcataagtcacgtggggagggctgcgtcacaaccaggaagcaatcctcaccacgtgatttgtcacgtgatcgctacgtcacggccgccattttagtttacaaaatggcggacttccttcctctttttcaaaaataacggccctgcggcggcgcgcgcgctgcgcgcgcgcgccgggggctgccgcccca

SEQ ID NO: 871 - protein, 121 aa, Alphatorquevirus sp.
MSWRPPVHNPNGIQRNLWEAFFRMHAAACGCGDLVGHITVLAGRYGAPPRPPAPGAPRPPLIRQLALPAPPADPQQANPQWPGGDGGEDGAGGPAAGGAVADAEYQEDELNALFDAVEQEE

SEQ ID NO: 872 - protein, 267 aa, Alphatorquevirus sp.
MSWRPPVHNPNGIQRNLWEAFFRMHAAACGCGDLVGHITVLAGRYGAPPRPPAPGAPRPPLIRQLALPAPPADPQQANPQWPGGDGGEDGAGGPAAGGAVADAEYQEDELNALFDAVEQEELLKTPAQTPGFDLQPVETVAQYKSLARSQWDPSLYSTHGTGDGGSLIKKLSKECLKNQLMMENIQQAQRCLDGFPHSTTKSKKASQIQRRQPRSPRKKKPLKKPSKKSKRRRYSSTSSSSTESSEESESNSNSSCSNSPRRRATCT

SEQ ID NO: 873 - protein, 277 aa, Alphatorquevirus sp.
MSWRPPVHNPNGIQRNLWEAFFRMHAAACGCGDLVGHITVLAGRYGAPPRPPAPGAPRPPLIRQLALPAPPADPQQANPQWPGGDGGEDGAGGPAAGGAVADAEYQEDELNALFDAVEQEEISNRPKGASMVSPTRQPRARRRLRYRDDNLAVLARRSRSRSPPRSPRGVATAAPPPAVPRAAKNRKATPTRHAPTHQDAEQPAHKSPCPWPCINKVYMFPPEKPMPIHGYHGWETEYQACKAFDRPPRNYLSDKPIYPWLPRSQPEFKVSFKLGCQ

SEQ ID NO: 874 - protein, 200 aa, Alphatorquevirus sp.
MSWRPPVHNPNGIQRNLWEAFFRMHAAACGCGDLVGHITVLAGRISNRPKGASMVSPTRQPRARRRLRYRDDNLAVLARRSRSRSPPRSPRGVATAAPPPAVPRAAKNRKATPTRHAPTHQDAEQPAHKSPCPWPCINKVYMFPPEKPMPIHGYHGWETEYQACKAFDRPPRNYLSDKPIYPWLPRSQPEFKVSFKLGCQ

SEQ ID NO: 875 - protein, 765 aa, Alphatorquevirus sp.
MAWWGRWRRWRWRPRRWRRRRRRRVPRRRAQRPVRRRRARRVRRRRWGRRRWRRGYRRRLRLRRRRRRKKIRLTQWNPAKVRRCTIKGVLPMILCGAGRSGFNYGLHSDDYTVQKPLGQNPHGGGMSTVTFSLQVLFDQYQRFMNRWSYSNDQLDLARYFGCTFYFYRHPEIDFVAQYDNVPPMKMDENTAPNTHPSFLLQNKRKIKIPSFKTKPFGRKRVKVTVGPPKLFEDKWYSQHDLCKVPLVSWRLTACDFRFPFCSPLTDNPCYTFQVLHENYYPVIGTSSLENGTNYNNTAITTLETWLYGKCTHYQTFATDTRLNPQRPVSSSNANETYTPSGSKESIIWGQSDWANFKKNTDSNYGYCSYCPSNGTNGTVDKIKKYRDQRFRWLTEMPVPNTCHIHATFARGTIKYWEYHLGWYSNIFIGNLRHNLAFRPAYIDITYNPITDKGEGNIIWFQYLTKPTTEYIETQAKCTITNIPLYAAFYGYEDYLQRTLGPYQDVETLGIICVKCPYTDPPLVHKDKSKTNWGYVFYDAHFGNGKTPEGLGQVHPYWMQRWRPYVQFQKDTMHKISRTGPFSYRDDTPSITLTAEYKFRFNWGGDSIFPQIIKNPCPDTGVRPSTGRDRRSVQVVSPLTMGPQFIFHSWDWRRGFFNQKTLKRMLEKPVNDGEYPTGPKVPRWFPPLDNQEQEGVSDTETTTSQSSQEEAAQEALQEVQEASLQQHLLQQYREQRRIGKQLQLVMLQLTKTQSNLHINPRVLGHA

SEQ ID NO: 876 - protein, 196 aa, Alphatorquevirus sp.
MAWWGRWRRWRWRPRRWRRRRRRRVPRRRAQRPVRRRRARRIIKNPCPDTGVRPSTGRDRRSVQVVSPLTMGPQFIFHSWDWRRGFFNQKTLKRMLEKPVNDGEYPTGPKVPRWFPPLDNQEQEGVSDTETTTSQSSQEEAAQEALQEVQEASLQQHLLQQYREQRRIGKQLQLVMLQLTKTQSNLHINPRVLGHA

SEQ ID NO: 877 - protein, 125 aa, Alphatorquevirus sp.
MAWWGRWRRWRWRPRRWRRRRRRRVPRRRAQRPVRRRRARRNIQQAQRCLDGFPHSTTKSKKASQIQRRQPRSPRKKKPLKKPSKKSKRRRYSSTSSSSTESSEESESNSNSSCSNSPRRRATCT

SEQ ID NO: 878 - DNA, 3264 bp, Gammatorquevirus sp.
  source 1..3264: mol_type = genomic DNA
taaaatggcggcaaccaatcattttatactttcactttccaattacaagccgccacgtcacagaacaggggtggagactttaaaactatataaccaagtgatgtgacgaatggctgagtttaccccgctagacggtgcagggaccggatcgagcgcagcgaggaggtccccggctgcccgtgggcgggagcccgaggtgagtgaaaccaccgaggtctaggggcaattcgggctagggcagtctagcggaacgggcaagaaacttaaaatatgttttgtttcagatgcagacacctgcttcacagataagctcagacgacttctttgtacacactccatttaatgcagtaactaaacagcaaatatggatgtctcaaattgctgatggacatgacaacatttgtcactgccaccgtccttttgctcacctgcttgctaatatttttcctcctggtcataaagacagggatcttaccattaatcaaatacttgctagagatcttacagaaacatgccattctggtggagacgaaggaacaagcggtggtggggtcgccgcttccgctaccgccgctacaacaaatataaaaccagaaggagacgcagaatacccagaagacgaaatagaagatttactaagacacgcaggagaagaaaaagaaagaaggtaagaagaaaacttaaaaaaattactattaaacaatggcagccagattcagtgaaaaaatgtaaaattaaaggatatagtactttagttatgggtgcacaaggaaaacaatacaactgttacacaaaccaagcaagtgactatgttcagcctaaagcaccacaaggtgggggctttggctgtgaagtatttaatttaaaatggctataccaagaatatactgcacacagaaatatttggacaaaaacaaatgaatatacagacctttgtagatacactggagctcaaataattttatacaggcacccagatgttgattttatagtcagctgggacaatcagccaccttttttacttaacaaatatacatatccagaactgcaaccacaaaaccttttactagctagaaggaaaagaattattcttagtcaaaaatcaaaccccaaaggaaaactaagaattaaactaagaataccaccaccaaaacaaatgataacaaaatggttttttcaaagagacttttgtgatgtgaatctgtttaaactatgtgcttctgctgcttctttccgctacccaggtatcagtcatggagctcaaagtactattttttctgcatatgctttaaacactgacttttatcaatgcagtgactggtgccaaactaacacagaaactggctacctaaacattaaaacacaacaaatgccactatggtttcattacagagagggtggcaaagagaaatggtataaatacaccaacaaagaacacagaccatatacaaatacatatcttaaaagtattagctataatgatggattgttttctcctaaagccatgtttgcatttgaagtaaaagcggggggtgaaggaacaacagaaccaccacaaggcgcccaattaattgctaaccttccactcattgcactaagatataatccacatgaagacacaggccatggcaatgaaatttaccttacatcaacttttaaaggtacatatgacaaacctaaagttactgatgctctatactttaacaatgtacccctgtggatgggattttatggctactgggactttatattacaagaaacaaaaaacaaaggtgtctttgatcaacatatgtttgttgttaaatgtcctgccttaaggcccatatcacaagtcacaaaacaagtatactacccacttgtagacatggacttttgttcagggagactgccatttgatgaatatttatccaaagacattaaaagtcattggtatcccactgcagaaagacaaacagttacaataaataattttgttacagcaggtccatacatgcctaaatttgaacccacagacaaagacagtacatggcaattaaactatcactataaatttttttttaagtggggtggtccacaagtcacagacccaactgttgaagacccatgcagcagaaacaaatatcctgtccccgatacaatgcaacaaacaatacaaattaaaaaccctgaaaagctgcacccagcaaccctcttccatgactgggaccttagaaggggcttcattacacaagcagctattaaaagaatgtcagaaaacctccaaattgattcatctttcgaatctgatggcacagaatcacccaaaaaaaagaaaagatgcaccaaagaaatcccaacacaaaaccaaaagcaagaagagatccaagaatgtctcctctcactctgcgaagagcctacatgccaagaagaaacagaggacctccagctcttcatccagcagcagcagcagcagcagtacaagctcagaaaaaacctcttcaaactcctcactcacctgaaaaaaggacagagaataagtcaactacaaacgggacttttagagtaataccatttaaaccaggttttgaacaagaaacagaaaaagaacttgccatagctttctgcagaccacctagaaaatataaaaatgatccccctttttatccctggttaccatggacaccccttgtacactttaaccttaattacaaaggctaggccaacactgttcacttagtggtgtatgtttaataaagtttcacccccaaaaaaaaaaaaaaaaaaaaaaaaaaaaaaaaaaataaaaaattgcaaaaattcggcgctcgcgcgcgctgcgcgcgcgcgagcgccgtcacgcgccggcgctcgcgcgccgcgcgtatgtgctaacacaccacgcacctagattggggtgcgcgcgctagcgcgcgcaccccaatgcgccccgccctcgttccgacccgcttgcgcgggtcggaccacttcgggctcgggggggcgcgcctgcggcgcttttttactaaacagactccgagccgccatttggccccccctaagctccgcccccctcatgaatattcataaaggaaaccacataattagaattgccgaccacaaactgccatatgctaattagttccccttttacacagtaaaaaggggaagtgggggggcatagcccccccacaccccccgcggggggggcagagcccccccccgcaccccccccctacgtcacaatccacgcccccgccgccatcttgggtgcggcagggcgggggc

SEQ ID NO: 879 - protein, 128 aa, Gammatorquevirus sp.
MQTPASQISSDDFFVHTPFNAVTKQQIWMSQIADGHDNICHCHRPFAHLLANIFPPGHKDRDLTINQILARDLTETCHSGGDEGTSGGGVAASATAATTNIKPEGDAEYPEDEIEDLLRHAGEEKERR

SEQ ID NO: 880 - protein, 272 aa, Gammatorquevirus sp.
MQTPASQISSDDFFVHTPFNAVTKQQIWMSQIADGHDNICHCHRPFAHLLANIFPPGHKDRDLTINQILARDLTETCHSGGDEGTSGGGVAASATAATTNIKPEGDAEYPEDEIEDLLRHAGEEKERSGVVHKSQTQLLKTHAAETNILSPIQCNKQYKLKTLKSCTQQPSSMTGTLEGASLHKQLLKECQKTSKLIHLSNLMAQNHPKKRKDAPKKSQHKTKSKKRSKNVSSHSAKSLHAKKKQRTSSSSSSSSSSSSTSSEKTSSNSSLT

SEQ ID NO: 881 - protein, 261 aa, Gammatorquevirus sp.
MQTPASQISSDDFFVHTPFNAVTKQQIWMSQIADGHDNICHCHRPFAHLLANIFPPGHKDRDLTINQILARDLTETCHSGGDEGTSGGGVAASATAATTNIKPEGDAEYPEDEIEDLLRHAGEEKERRITQKKEKMHQRNPNTKPKARRDPRMSPLTLRRAYMPRRNRGPPALHPAAAAAAVQAQKKPLQTPHSPEKRTENKSTTNGTFRVIPFKPGFEQETEKELAIAFCRPPRKYKNDPPFYPWLPWTPLVHFNLNYKG

SEQ ID NO: 882 - protein, 67 aa, Gammatorquevirus sp.
MDMTTFVTATVLLLTCLLIFFLLVIKTGILPLIKYLLEILQKHAILVETKEQAVVGSPLPLPPLQQI

SEQ ID NO: 883 - protein, 677 aa, Gammatorquevirus sp.
MPFWWRRRNKRWWGRRFRYRRYNKYKTRRRRRIPRRRNRRFTKTRRRRKRKKVRRKLKKITIKQWQPDSVKKCKIKGYSTLVMGAQGKQYNCYTNQASDYVQPKAPQGGGFGCEVFNLKWLYQEYTAHRNIWTKTNEYTDLCRYTGAQIILYRHPDVDFIVSWDNQPPFLLNKYTYPELQPQNLLLARRKRIILSQKSNPKGKLRIKLRIPPPKQMITKWFFQRDFCDVNLFKLCASAASFRYPGISHGAQSTIFSAYALNTDFYQCSDWCQTNTETGYLNIKTQQMPLWFHYREGGKEKWYKYTNKEHRPYTNTYLKSISYNDGLFSPKAMFAFEVKAGGEGTTEPPQGAQLIANLPLIALRYNPHEDTGHGNEIYLTSTFKGTYDKPKVTDALYFNNVPLWMGFYGYWDFILQETKNKGVFDQHMFVVKCPALRPISQVTKQVYYPLVDMDFCSGRLPFDEYLSKDIKSHWYPTAERQTVTINNFVTAGPYMPKFEPTDKDSTWQLNYHYKFFFKWGGPQVTDPTVEDPCSRNKYPVPDTMQQTIQIKNPEKLHPATLFHDWDLRRGFITQAAIKRMSENLQIDSSFESDGTESPKKKKRCTKEIPTQNQKQEEIQECLLSLCEEPTCQEETEDLQLFIQQQQQQQYKLRKNLFKLLTHLKKGQRISQLQTGLLE

SEQ ID NO: 884 - protein, 212 aa, Gammatorquevirus sp.
MPFWWRRRNKRWWGRRFRYRRYNKYKTRRRRRIPRRRNRRFTKTRRRRKRKKWGGPQVTDPTVEDPCSRNKYPVPDTMQQTIQIKNPEKLHPATLFHDWDLRRGFITQAAIKRMSENLQIDSSFESDGTESPKKKKRCTKEIPTQNQKQEEIQECLLSLCEEPTCQEETEDLQLFIQQQQQQQYKLRKNLFKLLTHLKKGQRISQLQTGLLE

SEQ ID NO: 885 - protein, 119 aa, Gammatorquevirus sp.
MPFWWRRRNKRWWGRRFRYRRYNKYKTRRRRRIPRRRNRRFTKTRRRRKRKKNHPKKRKDAPKKSQHKTKSKKRSKNVSSHSAKSLHAKKKQRTSSSSSSSSSSSSTSSEKTSSNSSLT

SEQ ID NO: 886 - DNA, 3176 bp, Gammatorquevirus sp.
  source 1..3176: mol_type = genomic DNA
taaaatggcgggagccaatcattttatactttcactttccaattaaaaatggccacgtcacaaacaaggggtggagccatttaaactatataactaagtggggtggcgaatggctgagtttaccccgctagacggtgcagggaccggatcgagcgcagcgaggaggtccccggctgcccatgggcgggagccgaggtgagtgaaaccaccgaggtctaggggcaattcgggctagggcagtctagcggaacgggcaagaaacttaaaacaatatttgttttacagatggttagtatatcctcaagtgatttttttaagaaaacgaaatttaatgaggagacgcagaaccaagtatggatgtctcaaattgctgactctcatgataatatctgcagttgctggcatccatttgctcaccttcttgcttccatatttcctcctggccacaaagatcgtgatcttactattaaccaaattcttctaagagattataaagaaaaatgccattctggtggagaagaaggagaaaattctggaccaacaacaggtttaattacaccaaaagaagaagatatagaaaaagatggcccagaaggcgccgcagaagaagaccatacagacgccctgttcgccgccgccgtagaaaacttcgaaaggtaaagagaaaaaaaaaatctttaattgttagacaatggcaaccagacagtataagaacttgtaaaattataggacagtcagctatagttgttggggctgaaggaaagcaaatgtactgttatactgtcaataagttaattaatgtgcccccaaaaacaccatatgggggaggctttggagtagaccaatacacactgaaatacttatatgaagaatacagatttgcacaaaacatttggacacaatctaatgtactgaaagacttatgcagatacataaatgttaagctaatattctacagagacaacaaaacagactttgtcctttcctatgacagaaacccaccttttcaactaacaaaatttacatacccaggagcacacccacaacaaatcatgcttcaaaaacaccacaaattcatactatcacaaatgacaaagcctaatggaagactaacaaaaaaactcaaaattaaacctcctaaacaaatgctttctaaatggttcttttcaaaacaattctgtaaataccctttactatctcttaaagcttctgcactagaccttaggcactcttacctaggctgctgtaatgaaaatccacaggtatttttttattatttaaaccatggatactacacaataacaaactggggagcacaatcctcaacagcatacagacctaactccaaggtgacagacacaacatactacagatacaaaaatgacagaaaaaatattaacattaaaagccatgaatacgaaaaaagtatatcatatgaaaacggttattttcaatctagtttcttacaaacacagtgcatatataccagtgagcgtggtgaagcctgtatagcagaaaaaccactaggaatagctatttacaatccagtaaaagacaatggagatggtaatatgatataccttgtaagcactctagcaaacacttgggaccagcctccaaaagacagtgctattttaatacaaggagtacccatatggctaggcttatttggatatttagactactgtagacaaattaaagctgacaaaacatggctagacagtcatgtactagtaattcaaagtcctgctatttttacttacccaaatccaggagcaggcaaatggtattgtccactatcacaaagttttataaatggcaatggtccgtttaatcaaccacctacactgctacaaaaagcaaagtggtttccacaaatacaataccaacaagaaattattaatagctttgtagaatcaggaccatttgttcccaaatatgcaaatcaaactgaaagcaactgggaactaaaatataaatatgtttttacatttaagtggggtggaccacaattccatgaaccagaaattgctgaccctagcaaacaagagcagtatgatgtccccgatactttctaccaaacaatacaaattgaagatccagaaggacaagaccccagatctctcatccatgattgggactacagacgaggctttattaaagaaagatctcttaaaagaatgtcaacttacttctcaactcatacagatcagcaagcaacttcagaggaagacattcccaaaaagaaaaagagaattggaccccaactcacagtcccacaacaaaaagaagaggagacactgtcatgtctcctctctctctgcaaaaaagataccttccaagaaacagagacacaagaagacctccagcagctcatcaagcagcagcaggagcagcagctcctcctcaagagaaacatcctccagctcatccacaaactaaaagagaatcaacaaatgcttcagcttcacacaggcatgttaccttaaccagatttaaacctggatttgaagagcaaacagagagagaattagcaattatatttcataggccccctagaacctacaaagaggaccttccattctatccctggctaccacctgcaccccttgtacaatttaaccttaacttcaaaggctaggccaacaatgtacacttagtaaagcatgtttattaaagcacaacccccaaaataaatgtaaaaataaaaaaaaaaaaaaaaaaataaaaaattgcaaaaattcggcgctcgcgcgcatgtgcgcctctggcgcaaatcacgcaacgctcgcgcgcccgcgtatgtctctttaccacgcacctagattggggtgcgcgcgctagcgcgcgcaccccaatgcgccccgccctcgttccgacccgcttgcgcgggtcggaccacttcgggctcgggggggcgcgcctgcggcgcttttttactaaacagactccgagccgccatttggccccctaagctccgcccccctcatgaatattcataaaggaaaccacataattagaattgccgaccacaaactgccatatgctaattagttccccttttacaaagtaaaaggggaagtgaacatagccccacacccgcaggggcaaggccccgcacccctacgtcactaaccacgcccccgccgccatcttgggtgcggcagggcgggggc

SEQ ID NO: 887 - protein, 124 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFER

SEQ ID NO: 888 - protein, 271 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFESGVDHNSMNQKLLTLANKSSMMSPILSTKQYKLKIQKDKTPDLSSMIGTTDEALLKKDLLKECQLTSQLIQISKQLQRKTFPKRKRELDPNSQSHNKKKRRHCHVSSLSAKKIPSKKQRHKKTSSSSSSSSRSSSSSSRETSSSSSTN

SEQ ID NO: 889 - protein, 267 aa, Gammatorquevirus sp.
MVSISSSDFFKKTKFNEETQNQVWMSQIADSHDNICSCWHPFAHLLASIFPPGHKDRDLTINQILLRDYKEKCHSGGEEGENSGPTTGLITPKEEDIEKDGPEGAAEEDHTDALFAAAVENFERSASNFRGRHSQKEKENWTPTHSPTTKRRGDTVMSPLSLQKRYLPRNRDTRRPPAAHQAAAGAAAPPQEKHPPAHPQTKRESTNASASHRHVTLTRFKPGFEEQTERELAIIFHRPPRTYKEDLPFYPWLPPAPLVQFNLNFKG

SEQ ID NO: 890 - protein, 50 aa, Gammatorquevirus sp.
MRRRRTKYGCLKLLTLMIISAVAGIHLLTFLLPYFLLATKIVILLLTKFF

SEQ ID NO: 891 - protein, 662 aa, Gammatorquevirus sp.
MPFWWRRRRKFWTNNRFNYTKRRRYRKRWPRRRRRRRPYRRPVRRRRRKLRKVKRKKKSLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYLNHGYYTITNWGAQSSTAYRPNSKVTDTTYYRYKNDRKNINIKSHEYEKSISYENGYFQSSFLQTQCIYTSERGEACIAEKPLGIAIYNPVKDNGDGNMIYLVSTLANTWDQPPKDSAILIQGVPIWLGLFGYLDYCRQIKADKTWLDSHVLVIQSPAIFTYPNPGAGKWYCPLSQSFINGNGPFNQPPTLLQKAKWFPQIQYQQEIINSFVESGPFVPKYANQTESNWELKYKYVFTFKWGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 892 - protein, 215 aa, Gammatorquevirus sp.
MPFWWRRRRKFWTNNRFNYTKRRRYRKRWPRRRRRRRPYRRPVRRRRRKLRKWGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 893 - protein, 129 aa, Gammatorquevirus sp.
MPFWWRRRRKFWTNNRFNYTKRRRYRKRWPRRRRRRRPYRRPVRRRRRKLRKISKQLQRKTFPKRKRELDPNSQSHNKKKRRHCHVSSLSAKKIPSKKQRHKKTSSSSSSSSRSSSSSSRETSSSSSTN

SEQ ID NO: 894 - DNA, 3696 bp, Alphatorquevirus sp.
  source 1..3696: mol_type = genomic DNA
attttgttcagcccgccaatttctctttcaaacaggccaatcagctactacttcgtgcacttcctggggcgtgtcctgccgctctatataagcagaggcggtgacgaatggtagagtttttcttggcccgtccgcggcgagagcgcgagcgaagcgagcgatcgagcgtcccgagggcgggtgccggaggtgagtttacacaccgcagtcaaggggcaattcgggctcgggactggccgggctatgggcaagattcttaaaaaattcccccgatccctttgccgccaggacataaaaacatgccgtggagaccgccggtccatagtgtccaggggcgagaggatcagtggttcgcaagcttttttcacggccacgattcgttttgcggctgcggtgaccctcttggccatattaatagcattgctcatcgctttcctcgcgccggtccaccaaggccccctccggggctagatcagcctaacccccgggagcagggcccggccggacccggagggccgcccgccatcttggccctgccggctccgcccgcggagcctgacgacccgcagccacggcgtggtggtggggacggtggcgccgccgctggcgccgcagacgaccatacacaacgagactacgacgaagaagagctagacgagcttttccgcgccgccgccgaagacgatttgtaagtaggagatggcgccggccttacaggcgcaggaggagacgcgggcgacgcagacgcagacgcagacgcagacataagcccaccctaatactcagacagtggcaacctgactgtatcagacactgtaaaataacaggatggatgcccctcattatctgtggaaaggggtccacccagttcaactacatcacccacgcggacgatatcacccccaggggagcctcctacggaggcaatttcacaaacatgactttctccctggaggccatatatgaacagttcctataccacagaaacaggtggtcggcctctaaccacgacctagaactgtgcagatacaaggggaccaccttaaaactctacagacacccagaagtagactacatagttacctacagcagaacaggaccctttgaaatcagccacatgacctacctcagcactcaccccatgctaatgctgctaaacaagcaccacattgtggtgcccagcttaaagactaagcccagaggcagaaaggccataaaagtcaggataaggcccccaaaactcatgaacaacaagtggtacttcaccagagacttctgtaacataggcctcttccagctctgggccacaggcttagaactcagaaacccctggctcagaatgagcaccctgagcccctgcataggctttaatgtcctcaaaaacagcatttacacaaacctcagcaacctgccacaatacaaaaacgaaagactaaacatcattaacaacatacttcacccacaagaaattacaggtacaaacaacaaaaagtggcagtacacatacaccaaactcatggcccctatttactattcagcaaacagggccagcacctatgactgggaaaattacagcaaagaaacaaactacaataatacatatgttaaatttacccagaaaagacaggaaaaactaactaaaattagaaaagagtggcagatgctttatccacaacaacccacagcactgccagactcctatgacctcctacaagagtatggcctctacagtccatactacctaaaccccacaagaataaacctagactggatgaccccatacacacacgtcagatacaatcccctagtagacaagggctttggaaacagaatatacatccagtggtgctcagaagcagatgttagctacaacaggacaaaatccaagtgtctgctacaagacatgcccctgtttttcatgtgctatggctacatagactgggcaataaaaaacactggagtgtcatctctagtgaaggacgccagaatctgcatcaggtgtccctacacagagccacaactagttggctccacagaagacataggctttgtacccatctcagaaaccttcatgaggggcgacatgccggtacttgcaccatacataccgttaagctggttttgcaagtggtatcccaacatagctcaccaaaaggaagtccttgagtcaatcatttcctgcagccccttcatgccccgtgaccaagacatgaacggttgggatatcacaatcggttacaaaatggacttcttatggggcggttcccctctcccctcacagccaatcgacgacccctgccagcagggaacccacccgattcccgaccccgataaacaccctcgcctcctacaagtctcgaacccgaaactactcggaccgaggacagtgttccacaagtgggacatcagacgtgggcagtttagcaaaagaagtattaagagagtgtcagaatactcaagcgatgatgaatctcttgcgccaggtctcccatcaaagcgaaacaagctcgactcggcgttccgaggagaaaatcgagagcaaaaagaatgctattctctcctcaaagcgctcgaggaagaagagaccccagaagaagaagaaccagcaccccaagaaaaagcccagaaagaggagctactccaccagctccagctccagagacgccaccagcgagtcctcagacgagggctcaagctcgtctttacagacatcctccgactccgccagggagtccactggaacccggagctcacatagcgcccccaccttacataccagacctgctttttcccaatactggtaaaaaaaaaaaattctctcccttcgattgggagacagaggcgcaaatagcggggtggatgcggcggcccatgcgcttctatccctcagacacccctcactacccgtggctaccccccgagcgagatatcccgaaaatatgtaacataaacttcaaaataaagcttcaagagtgagtgattcgaggccctcctctgttcacttagcggtgtctacctcttaaggtcactaagcactccgagcgtaagcgaggagtgcgaccctctaccaaggggcaacttcctcggggtccggcgctacgcgcttcgcgctgcgccggacatctcggacccctcgacccgaatcgcttgcgcgattcggacctgcggcctcgggggggtcgggggctttactaaacagactccgaggtgccattggacactgtagggggtgaacagcaacgaaagtgagtggggccagacttcgccataaggcctttatcttcttgccattggatagtgacttccgggtccgcctgggggccgccattttagcttcggccgccattttaggccctcgcgggcctccgtaggcgcgctttagtgacgtcacggcagccattttgtcgtgacgtttgagacacgtgatgggggcgtgcctaaacccggaagcatccctggtcacgtgactctgacgtcacggcggccatcttgtgctgtccgccatcttgtaacttccttccgctttttcaaaaaaaaagaggaagtgtgacgtagcggcgggggggcggcgcgcttcgcgcgccgcccaccagggggcgctgcgcgccccccgcgcatgcgcaggggcctctcgaggggctccgccccccccccgtgctaaatttaccgcgcatgcgcgaccacgcccccgccgcc

SEQ ID NO: 895 - protein, 130 aa, Alphatorquevirus sp.
MPWRPPVHSVQGREDQWFASFFHGHDSFCGCGDPLGHINSIAHRFPRAGPPRPPPGLDQPNPREQGPAGPGGPPAILALPAPPAEPDDPQPRRGGGDGGAAAGAADDHTQRDYDEEELDELFRAAAEDDL

SEQ ID NO: 896 - protein, 303 aa, Alphatorquevirus sp.
MPWRPPVHSVQGREDQWFASFFHGHDSFCGCGDPLGHINSIAHRFPRAGPPRPPPGLDQPNPREQGPAGPGGPPAILALPAPPAEPDDPQPRRGGGDGGAAAGAADDHTQRDYDEEELDELFRAAAEDDFQSTTPASREPTRFPTPINTLASYKSRTRNYSDRGQCSTSGTSDVGSLAKEVLRECQNTQAMMNLLRQVSHQSETSSTRRSEEKIESKKNAILSSKRSRKKRPQKKKNQHPKKKPRKRSYSTSSSSRDATSESSDEGSSSSLQTSSDSARESTGTRSSHSAPTLHTRPAFSQYW

SEQ ID NO: 897 - protein, 292 aa, Alphatorquevirus sp.
MPWRPPVHSVQGREDQWFASFFHGHDSFCGCGDPLGHINSIAHRFPRAGPPRPPPGLDQPNPREQGPAGPGGPPAILALPAPPAEPDDPQPRRGGGDGGAAAGAADDHTQRDYDEEELDELFRAAAEDDLSPIKAKQARLGVPRRKSRAKRMLFSPQSARGRRDPRRRRTSTPRKSPERGATPPAPAPETPPASPQTRAQARLYRHPPTPPGSPLEPGAHIAPPPYIPDLLFPNTGKKKKFSPFDWETEAQIAGWMRRPMRFYPSDTPHYPWLPPERDIPKICNINFKIKLQ

SEQ ID NO: 898 - protein, 180 aa, Alphatorquevirus sp.
MPWRPPVHSVQGREDQWSPIKAKQARLGVPRRKSRAKRMLFSPQSARGRRDPRRRRTSTPRKSPERGATPPAPAPETPPASPQTRAQARLYRHPPTPPGSPLEPGAHIAPPPYIPDLLFPNTGKKKKFSPFDWETEAQIAGWMRRPMRFYPSDTPHYPWLPPERDIPKICNINFKIKLQE

SEQ ID NO: 899 - protein, 49 aa, Alphatorquevirus sp.
IVSRGERISGSQAFFTATIRFAAAVTLLAILIALLIAFLAPVHQGPLRG

SEQ ID NO: 900 - protein, 728 aa, Alphatorquevirus sp.
TAWWWGRWRRRWRRRRPYTTRLRRRRARRAFPRRRRRRFVSRRWRRPYRRRRRRGRRRRRRRRRHKPTLILRQWQPDCIRHCKITGWMPLIICGKGSTQFNYITHADDITPRGASYGGNFTNMTFSLEAIYEQFLYHRNRWSASNHDLELCRYKGTTLKLYRHPEVDYIVTYSRTGPFEISHMTYLSTHPMLMLLNKHHIVVPSLKTKPRGRKAIKVRIRPPKLMNNKWYFTRDFCNIGLFQLWATGLELRNPWLRMSTLSPCIGFNVLKNSIYTNLSNLPQYKNERLNIINNILHPQEITGTNNKKWQYTYTKLMAPIYYSANRASTYDWENYSKETNYNNTYVKFTQKRQEKLTKIRKEWQMLYPQQPTALPDSYDLLQEYGLYSPYYLNPTRINLDWMTPYTHVRYNPLVDKGFGNRIYIQWCSEADVSYNRTKSKCLLQDMPLFFMCYGYIDWAIKNTGVSSLVKDARICIRCPYTEPQLVGSTEDIGFVPISETFMRGDMPVLAPYIPLSWFCKWYPNIAHQKEVLESIISCSPFMPRDQDMNGWDITIGYKMDFLWGGSPLPSQPIDDPCQQGTHPIPDPDKHPRLLQVSNPKLLGPRTVFHKWDIRRGQFSKRSIKRVSEYSSDDESLAPGLPSKRNKLDSAFRGENREQKECYSLLKALEEEETPEEEEPAPQEKAQKEELLHQLQLQRRHQRVLRRGLKLVFTDILRLRQGVHWNPELT

SEQ ID NO: 901 - protein, 197 aa, Alphatorquevirus sp.
TAWWWGRWRRRWRRRRPYTTRLRRRRARRAFPRRRRRRFPIDDPCQQGTHPIPDPDKHPRLLQVSNPKLLGPRTVFHKWDIRRGQFSKRSIKRVSEYSSDDESLAPGLPSKRNKLDSAFRGENREQKECYSLLKALEEEETPEEEEPAPQEKAQKEELLHQLQLQRRHQRVLRRGLKLVFTDILRLRQGVHWNPELT

SEQ ID NO: 902 - protein, 145 aa, Alphatorquevirus sp.
TAWWWGRWRRRWRRRRPYTTRLRRRRARRAFPRRRRRRFVSHQSETSSTRRSEEKIESKKNAILSSKRSRKKRPQKKKNQHPKKKPRKRSYSTSSSSRDATSESSDEGSSSSLQTSSDSARESTGTRSSHSAPTLHTRPAFSQYW

SEQ ID NO: 903 - DNA, 3828 bp, Alphatorquevirus sp.
  source 1..3828: mol_type = genomic DNA
gtgctacgtcactaacctacgtgtccgtctcccataggccggacaccgtatacgtcatacacttcctgggcatggtctacgtgataatataagtggctgcacttccgaatggctgagttttccacgcccgtccgcagcgaggacgccacggagggggatccgcgcgtcccgagggcgggtgccggaggtgagtttacacaccgcagtcaaggggcaattcgggctcgggactggccgggctatgggcaaggctcttaaaaatgcacttttctaggtgcagtagaaagaaaaggacattgtcactgctaccactgtaccattcacagaaagctaggccatctgtgacaggtatgtggagacccccgactcgaaatgcgttcaatattcaacgtgactggttctacagttgctttcactcccacgcttctatgtgcggctgtgctgattttattggtcatttcaatcatatcgctgctatgctcggccgtccggaagaccagaaccctcctccgccacccggggctctgagacccctacccgctctcccggcctcttccgaggcacccggtgatcgagcgccatggcctatgggtggtggcggaggcgacggaggcgcccgtggtggaggaggagatggcgccgctggagacgccgtcggagaccccgcagacgccgacctcgtcgccgctatcgacgccgcagaacagtaaggaggcgcggcagggggaggtggactagagcacacaggagatggcgccgcaagggaaaacgcagtcgcaaaaaaaagattattataagacaatggcagcccaactacactcgcagatgcaacatagtgggctacatgcctctactaatatgtggggaaaatactgttgctacaaactatgccacccactcagacgacagctactaccccggaccctttggggggggaatgactacagacaaatttactctaagaatactgtatgatgagtacaaaaggttcatgaactactggacctcttcaaacgaggacctagacctatgtagatacctgggatgcactctatatgtgtttagacacccagaagtagactttataatcattataaatacctctcctccattcctagacacagaaataacagggcctagcatacacccaggtatgatggcccttaacaaaagaagcagatggatacctagcataaaaaacagaccaggcagaaagcactatataaagattaaagtaggagccccccgaatgttcacagataagtggtacccccaaacagacctctgtgacatgacactcctaacgatctttgccagtgcggcggatatgcaatatccgttcggctcaccactaactgacaccatagttgtgtcattccaagttctgcaatccatgtacaacgactgcctgagtgtacttcctgataattttgcagagacatcaggcaaaggcacccaactacatgagaacataatacaacatctgccctactacaacaccacacaaacacaagcacaatttaaaagatttatagaaaacatgaatgcaacaaatggagacaatatatgggcaagctacataaacacaaccaagttctcatccgcaaacactccaaagaatgacacaggcataggaggcccttacactacatattcagactcatggtacaaaggcacagtatacaatgacaaaattaaaaccataccaataaaagcaagcaagttatactacgagcaaaccaaaaacctcattggcattacattcactggatccacacacagactccattactgtggaggcctatactcctccgtatggctatcagcaggtagatcctactttgaaaccaaaggcccatacacagacataacttacaaccccttttcagacagaggagagggtaacatgctatggatagactggctaactaaaaatgactcagtgtactcaaaaacaagtagcaagtgtcttatagaaaacctgcccctgtgggcctcagtatacggatataaagaatactgcagcaaggtaacaggagacacaaacatagaacacaactgtagatgtgttatcagaagcccctacacagtaccacaactgttagaccacaacaatcccttcagaggatacgtgccttatagcttcaactttggaaatggtaaaatgccaggcggtagcagcctagtgcccattagaatgagagccaagtggtaccccactctgttccaccaaaaagaagttctagaagccatagcacaggcgggccccttcgcataccactcagatattaaaaaagtgtccctgggcataaagtacagatttaagtgggtgtggggtggcaaccccgtgtcccaacaggttgttagaaacccctgcaagaccacccaaggttcctcgggcaatagagtgcctcgatcaatacaagtcgttgacccgcggtacaacacgccagaactcaccatacacgcgtgggacttcagacatgggttctttggcagaaaagctattaagagaatgcaagaacaaccaatacctcatgacactttttcagcagggttcaagcgcagtcgccgagatacagaagcactccaatgcagccaagaagagcaacaaaaagaaaacttacttttcccagtccagcagctcaagcgagtccccccgtgggagacctcgcaagagagccaaagcgaggaagaaaactcgcaaaaacaggagaccctctcccagcaactcagagaccagctgcacaagcagcggctcatgggagagcaactccgatcgctcctctaccaaatgcagagggtccaacaaaatcaacacataaaccctatgttattgccaaagggtctggcattaacttctatttctcacaatgtaatatagatatgtttggtgaccccaaaccctacaagccctcctccaatgactggaaggaggagtacgaggccgcaaagtactgggacagaccccccagacgcgacctgaggagcacccccttctacccctgggcccccacccccaaaccatacaatgtcaactttgccctcaactacaaataaacggtggccgtgggagtttcacttgtcggtgtctacctcttaaggtcactaagcactccgagcgtaagcgaggagtgcgacccttcaccaagggcaactccctcgaagtccggcgctacgcgcttcgcgctgcgccggacatctcggaccccccctcgacccgaatcgcttgcgcgattcggacctgcggcctcgggggggtcgggggctttactaaacagactccgaggtgccattggacactgagggggtgaacagcaacgaaagtgagtggggccagacttcgccataaggcctttatcttcttgccatttgtccgcgaccgggggtcgctcctaggcgcggaccccgtttcggggtccttccgggttcatcggcgccgttccagtgacgtcacgggcgccatgttaagtggctgtcgccgaggattgacgtcacagttcaaaggtcatcctcggcggtaaccgcaaacatggcggtcaatctcttccgggtcaaaggtcgtgcatacgtcataagtcacatgacaggggtccacttaaacacggaagtaggccccgacatgtgactcgtcacgtgtgtacacgtcacggccgccattttgttttacaaaatggccgacttccttcctgttttttaaaaaaaggcgcgaaaaaaccgtcggcgggggccgcgcgctgcgcgcgcgggaggcaatgcctccccccccccgcgcgcatgcgcgcgggtccccccccctccggggggctccgccccccggcccccccc

SEQ ID NO: 904 - protein, 119 aa, Alphatorquevirus sp.
MWRPPTRNAFNIQRDWFYSCFHSHASMCGCADFIGHFNHIAAMLGRPEDQNPPPPPGALRPLPALPASSEAPGDRAPWPMGGGGGDGGARGGGGDGAAGDAVGDPADADLVAAIDAAEQ

SEQ ID NO: 905 - protein, 273 aa, Alphatorquevirus sp.
MWRPPTRNAFNIQRDWFYSCFHSHASMCGCADFIGHFNHIAAMLGRPEDQNPPPPPGALRPLPALPASSEAPGDRAPWPMGGGGGDGGARGGGGDGAAGDAVGDPADADLVAAIDAAEQLLETPARPPKVPRAIECLDQYKSLTRGTTRQNSPYTRGTSDMGSLAEKLLRECKNNQYLMTLFQQGSSAVAEIQKHSNAAKKSNKKKTYFSQSSSSSESPRGRPRKRAKARKKTRKNRRPSPSNSETSCTSSGSWESNSDRSSTKCRGSNKINT

SEQ ID NO: 906 - protein, 286 aa, Alphatorquevirus sp.
MWRPPTRNAFNIQRDWFYSCFHSHASMCGCADFIGHFNHIAAMLGRPEDQNPPPPPGALRPLPALPASSEAPGDRAPWPMGGGGGDGGARGGGGDGAAGDAVGDPADADLVAAIDAAEQVQAQSPRYRSTPMQPRRATKRKLTFPSPAAQASPPVGDLAREPKRGRKLAKTGDPLPATQRPAAQAAAHGRATPIAPLPNAEGPTKSTHKPYVIAKGSGINFYFSQCNIDMFGDPKPYKPSSNDWKEEYEAAKYWDRPPRRDLRSTPFYPWAPTPKPYNVNFALNYK

SEQ ID NO: 907 - protein, 51 aa, Alphatorquevirus sp.
MRSIFNVTGSTVAFTPTLLCAAVLILLVISIISLLCSAVRKTRTLLRHPGL

SEQ ID NO: 908 - protein, 767 aa, Alphatorquevirus sp.
MAYGWWRRRRRRPWWRRRWRRWRRRRRPRRRRPRRRYRRRRTVRRRGRGRWTRAHRRWRRKGKRSRKKKIIIRQWQPNYTRRCNIVGYMPLLICGENTVATNYATHSDDSYYPGPFGGGMTTDKFTLRILYDEYKRFMNYWTSSNEDLDLCRYLGCTLYVFRHPEVDFIIIINTSPPFLDTEITGPSIHPGMMALNKRSRWIPSIKNRPGRKHYIKIKVGAPRMFTDKWYPQTDLCDMTLLTIFASAADMQYPFGSPLTDTIVVSFQVLQSMYNDCLSVLPDNFAETSGKGTQLHENIIQHLPYYNTTQTQAQFKRFIENMNATNGDNIWASYINTTKFSSANTPKNDTGIGGPYTTYSDSWYKGTVYNDKIKTIPIKASKLYYEQTKNLIGITFTGSTHRLHYCGGLYSSVWLSAGRSYFETKGPYTDITYNPFSDRGEGNMLWIDWLTKNDSVYSKTSSKCLIENLPLWASVYGYKEYCSKVTGDTNIEHNCRCVIRSPYTVPQLLDHNNPFRGYVPYSFNFGNGKMPGGSSLVPIRMRAKWYPTLFHQKEVLEAIAQAGPFAYHSDIKKVSLGIKYRFKWVWGGNPVSQQVVRNPCKTTQGSSGNRVPRSIQVVDPRYNTPELTIHAWDFRHGFFGRKAIKRMQEQPIPHDTFSAGFKRSRRDTEALQCSQEEQQKENLLFPVQQLKRVPPWETSQESQSEEENSQKQETLSQQLRDQLHKQRLMGEQLRSLLYQMQRVQQNQHINPMLLPKGLALTSISHNVI

SEQ ID NO: 909 - protein, 216 aa, Alphatorquevirus sp.
MAYGWWRRRRRRPWWRRRWRRWRRRRRPRRRRPRRRYRRRRTVVRNPCKTTQGSSGNRVPRSIQVVDPRYNTPELTIHAWDFRHGFFGRKAIKRMQEQPIPHDTFSAGFKRSRRDTEALQCSQEEQQKENLLFPVQQLKRVPPWETSQESQSEEENSQKQETLSQQLRDQLHKQRLMGEQLRSLLYQMQRVQQNQHINPMLLPKGLALTSISHNVI

SEQ ID NO: 910 - protein, 131 aa, Alphatorquevirus sp.
MAYGWWRRRRRRPWWRRRWRRWRRRRRPRRRRPRRRYRRRRTGSSAVAEIQKHSNAAKKSNKKKTYFSQSSSSSESPRGRPRKRAKARKKTRKNRRPSPSNSETSCTSSGSWESNSDRSSTKCRGSNKINT

SEQ ID NO: 911 - DNA, 3815 bp, Alphatorquevirus sp.
  source 1..3815: mol_type = genomic DNA
aagatcgtcactaaccacgtgactcctctcgcccaatcagtgtctacgtcgtccatttcctgggcatggtctacatcctgatataaagcgatgcacttccgaatggctgagttttccacgcccgtccgcggcgagatcgcgacggaggagcgatcgagcgtcccgagggcgggtgccggaggtgagtttacacaccgcagtcaaggggcaattcgggctcgggactggccgggctatgggcaaggctcttaaagcgtacgtcccccgctatgtttctcggcagggtgtggaggaaacagaaaaggaaagtgcttctgctggctgtgcgagctacacagaaaacatcttccatgagtatctggcgtcccccccttgggaatgtctcctacagggagagaaattggcttcaggccgtcgaaacatcccacagttctttttgtggctgtggtgattttattcttcatcttactaatttggctgcacgctttgctctccaggggcccccgccagagggtggtccacctcggccgaggccgccgctcctgagagcgctgccggcccccgaggtccgcagggagacgcgcacagagaaccggggcgcctccggtgagccatggcctggcgatggtggtggcagagacgatggcgccgccgccggtggccccgcagacggtggagacgcctacgacgccggagacctagacgacctgttcgccgccgtcgaagaagaacaacagtaaggaggcggaggtggaggggcagacgtgggcgacgcacatacacccgacgcgcggtcagacgcagacgcagacccagaaagagacttgtactgactcagtggagcccccagacagtcagaaactgctcaataaggggcatagtgcccatggtaatatgcggacacacaaaagcaggtagaaactatgctattcatagcgaggacttcaccacacagatacaacccttcgggggcagtttcagcacgaccacctggtccctaaaagtgctgtgggacgagcaccagaaattccagaacagatggtcctacccaaacacacaactagacctggccagatacagaggggtcaccttctggttctacagagaccagaaaacagactatatagtacagtggagtaggaatcccccttttaaactcaataaatacagcagtgccatgtaccacccgggcatgatgatgcaggccaaaaggaaactagttgtacctagtttccagaccagacccaaaggcaagaagagatacagagtcacaataaaaccccctaacatgtttgctgacaagtggtacactcaagaggacctgtgtccggtacctcttgtgcaaattgtggtttctgcggcgagcctgctacatccgttctgcccaccacaaacgaacaacccttgcatcaccttccaggttttgaaagacatatatgatgaatgcataggagttaacgaaactatgaaagataagtataagaaattacaaacaacactatacaccacttgcacatactatcaaacaacacaagtactggcacagctatctcctgcctttcaacctgctatgaaacctactactacacaatcagcagctacagcgacaacactaggaaactatgtaccagagttaaagtacaacaatggctcttttcacacaggacaaaacgcagtattcggcatgtgctcatacaaaccaacagacagcataatgacaaaagctaatggctggttttggcaaaacctaatggtagacaacaacctacatagttcttatggcaaggcaacattagaatgcatggagtatcacacaggcatatacagctctatatttctaagtccacaaagatctttagaattcccagcagcataccaagacgttacatacaaccctaactgtgatagagcagttggaaacgtagtttggtttcagtacagcactaaaatggatacaaattttgatgaaacaaaatgtaaatgtgtccttaaaaacattccactgtgggcggccttcaatggctactcagactttataatgcaagaactcagcataagtacagaaatccacaactttggcatagtgtgctttcagtgcccgtacacttttcccccctgtttcaataaaaacaaacccctaaaggggtacgtgttctatgacaccacctttggtaatggaaaaatgccagacggatcggggcacgtacccatctactggcagcagagatggtggatcagactagccttccaggtccaggtcatgcatgactttgtactaacaggcccctttagctacaaagatgacctagcaaacaccacactcacagccagatacaaatttaaattcaaatggggcggcaatatcatccctgaacagattatcaagaacccgtgtcacagagagcagtccctcgcttcctatcccgatagacaacgtcgcgacctacaagttgttgacccatcaaccatgggcccgatctacaccttccacacatgggactggcgacgggggctttttggtgcagatgctatccagagagtgtcacaaaaaccgggagatgctctccgctttacaaaccctttcaagagacccagatatcttcccccgacagacagagaagactaccgacaagaagaagacttcgctttacaggaaaaaagacggcgcacatccacagaagaagcccaggacgaggagagccccccggaaagcgcgccgctcctacagcagcagcagcagcagcggcagctctcagtccacctcgcggagcagcagcgactcggagtccaactccgatacatcctccaagaagtcctcaaaacgcaagcgggtctccacctaaaccccctattattaggcccgccacaaacaaggtctatctctttgagccctccaaaggcctactccccatagtaggaaaagaggcctgggaggacgagtactgcacctgcaagtactgggatcgccctcccagaaccaaccacctagacatccccacttatccctggatgcccacaaacttcaaagtcagcttcaaacttggatttaaaccctaaataaaaatacaaggccgtacactgttcacttgtcggtgtctacctctataagtcactaagcactccgagcgcagcgaggagtgcgaccctcagcggtgggtgcaacgccctcggcggccgcgcgctacgccttcggctgcgcgcggcacctcggacccccgctcgtgctgacacgctcgcgcgtgtcagaccacttcgggctcgcgggggtcgggaattttgctaaacagactccgagttgctcttggacactgtagctgtgaatcagtaacgaaagtgagtggggccagacttcgccataaggcctttatcttcttgccattggtccgtctcgggggtcgccataggcttcgggctcggttttaggccttccggactaccaaaatggcggattccgtgacgtcatggccgccattttaagtaaggcggaacaggctgtcaccccgtgtcaaagttcaggggtcagccttccgctttacacaaaatggaggtcaatatcttccgggtcaaaggtcgctaccgcgtcataagtcacgtggggaaggctgctgtgaatccggaagtagctgacccacgtgacttgtcacgtgactagcacgtcacggcagccattttgaatcacaaaatggccgacttccttcctcttttttaaaaataacggcccggcggcggcgcgcgcgcttcgcgccgctccgccccccccgcgcatgcgcgggacccccccccgcggggggctccgccccccggtcccccccccg

SEQ ID NO: 912 - protein, 129 aa, Alphatorquevirus sp.
MSIWRPPLGNVSYRERNWLQAVETSHSSFCGCGDFILHLTNLAARFALQGPPPEGGPPRPRPPLLRALPAPEVRRETRTENRGASGEPWPGDGGGRDDGAAAGGPADGGDAYDAGDLDDLFAAVEEEQQ

SEQ ID NO: 913 - protein, 283 aa, Alphatorquevirus sp.
MSIWRPPLGNVSYRERNWLQAVETSHSSFCGCGDFILHLTNLAARFALQGPPPEGGPPRPRPPLLRALPAPEVRRETRTENRGASGEPWPGDGGGRDDGAAAGGPADGGDAYDAGDLDDLFAAVEEEQQLSRTRVTESSPSLPIPIDNVATYKLLTHQPWARSTPSTHGTGDGGFLVQMLSRECHKNREMLSALQTLSRDPDIFPRQTEKTTDKKKTSLYRKKDGAHPQKKPRTRRAPRKARRSYSSSSSSGSSQSTSRSSSDSESNSDTSSKKSSKRKRVST

SEQ ID NO: 914 - protein, 305 aa, Alphatorquevirus sp.
MSIWRPPLGNVSYRERNWLQAVETSHSSFCGCGDFILHLTNLAARFALQGPPPEGGPPRPRPPLLRALPAPEVRRETRTENRGASGEPWPGDGGGRDDGAAAGGPADGGDAYDAGDLDDLFAAVEEEQQCYPESVTKTGRCSPLYKPFQETQISSPDRQRRLPTRRRLRFTGKKTAHIHRRSPGRGEPPGKRAAPTAAAAAAAALSPPRGAAATRSPTPIHPPRSPQNASGSPPKPPIIRPATNKVYLFEPSKGLLPIVGKEAWEDEYCTCKYWDRPPRTNHLDIPTYPWMPTNFKVSFKLGFKP

SEQ ID NO: 915 - protein, 55 aa, Alphatorquevirus sp.
MSPTGREIGFRPSKHPTVLFVAVVILFFILLIWLHALLSRGPRQRVVHLGRGRRS

SEQ ID NO: 916 - protein, 766 aa, Alphatorquevirus sp.
MAWRWWWQRRWRRRRWPRRRWRRLRRRRPRRPVRRRRRRTTVRRRRWRGRRGRRTYTRRAVRRRRRPRKRLVLTQWSPQTVRNCSIRGIVPMVICGHTKAGRNYAIHSEDFTTQIQPFGGSFSTTTWSLKVLWDEHQKFQNRWSYPNTQLDLARYRGVTFWFYRDQKTDYIVQWSRNPPFKLNKYSSAMYHPGMMMQAKRKLVVPSFQTRPKGKKRYRVTIKPPNMFADKWYTQEDLCPVPLVQIVVSAASLLHPFCPPQTNNPCITFQVLKDIYDECIGVNETMKDKYKKLQTTLYTTCTYYQTTQVLAQLSPAFQPAMKPTTTQSAATATTLGNYVPELKYNNGSFHTGQNAVFGMCSYKPTDSIMTKANGWFWQNLMVDNNLHSSYGKATLECMEYHTGIYSSIFLSPQRSLEFPAAYQDVTYNPNCDRAVGNVVWFQYSTKMDTNFDETKCKCVLKNIPLWAAFNGYSDFIMQELSISTEIHNFGIVCFQCPYTFPPCFNKNKPLKGYVFYDTTFGNGKMPDGSGHVPIYWQQRWWIRLAFQVQVMHDFVLTGPFSYKDDLANTTLTARYKFKFKWGGNIIPEQIIKNPCHREQSLASYPDRQRRDLQVVDPSTMGPIYTFHTWDWRRGLFGADAIQRVSQKPGDALRFTNPFKRPRYLPPTDREDYRQEEDFALQEKRRRTSTEEAQDEESPPESAPLLQQQQQQRQLSVHLAEQQRLGVQLRYILQEVLKTQAGLHLNPLLLGPPQTRSISLSPPKAYSP

SEQ ID NO: 917 - protein, 219 aa, Alphatorquevirus sp.
MAWRWWWQRRWRRRRWPRRRWRRLRRRRPRRPVRRRRRRTTIIKNPCHREQSLASYPDRQRRDLQVVDPSTMGPIYTFHTWDWRRGLFGADAIQRVSQKPGDALRFTNPFKRPRYLPPTDREDYRQEEDFALQEKRRRTSTEEAQDEESPPESAPLLQQQQQQRQLSVHLAEQQRLGVQLRYILQEVLKTQAGLHLNPLLLGPPQTRSISLSPPKAYSP

SEQ ID NO: 918 - protein, 146 aa, Alphatorquevirus sp.
MAWRWWWQRRWRRRRWPRRRWRRLRRRRPRRPVRRRRRRTTMLSRECHKNREMLSALQTLSRDPDIFPRQTEKTTDKKKTSLYRKKDGAHPQKKPRTRRAPRKARRSYSSSSSSGSSQSTSRSSSDSESNSDTSSKKSSKRKRVST

SEQ ID NO: 919 - protein, 677 aa, Gammatorquevirus sp.; identical to SEQ ID NO: 883

SEQ ID NO: 920 - protein, 59 aa, Gammatorquevirus sp.
MPFWWRRRNKRWWGRRFRYRRYNKYKTRRRRRIPRRRNRRFTKTRRRRKRKKVRRKLKK

SEQ ID NO: 921 - protein, 201 aa, Gammatorquevirus sp.
ITIKQWQPDSVKKCKIKGYSTLVMGAQGKQYNCYTNQASDYVQPKAPQGGGFGCEVFNLKWLYQEYTAHRNIWTKTNEYTDLCRYTGAQIILYRHPDVDFIVSWDNQPPFLLNKYTYPELQPQNLLLARRKRIILSQKSNPKGKLRIKLRIPPPKQMITKWFFQRDFCDVNLFKLCASAASFRYPGISHGAQSTIFSAYAL

SEQ ID NO: 922 - protein, 96 aa, Gammatorquevirus sp.
NTDFYQCSDWCQTNTETGYLNIKTQQMPLWFHYREGGKEKWYKYTNKEHRPYTNTYLKSISYNDGLFSPKAMFAFEVKAGGEGTTEPPQGAQLIAN

SEQ ID NO: 923 - protein, 161 aa, Gammatorquevirus sp.
LPLIALRYNPHEDTGHGNEIYLTSTFKGTYDKPKVTDALYFNNVPLWMGFYGYWDFILQETKNKGVFDQHMFVVKCPALRPISQVTKQVYYPLVDMDFCSGRLPFDEYLSKDIKSHWYPTAERQTVTINNFVTAGPYMPKFEPTDKDSTWQLNYHYKFFFK

SEQ ID NO: 924 - protein, 160 aa, Gammatorquevirus sp.
WGGPQVTDPTVEDPCSRNKYPVPDTMQQTIQIKNPEKLHPATLFHDWDLRRGFITQAAIKRMSENLQIDSSFESDGTESPKKKKRCTKEIPTQNQKQEEIQECLLSLCEEPTCQEETEDLQLFIQQQQQQQYKLRKNLFKLLTHLKKGQRISQLQTGLLE

SEQ ID NO: 925 - protein, 662 aa, Gammatorquevirus sp.; identical to SEQ ID NO: 891

SEQ ID NO: 926 - protein, 58 aa, Gammatorquevirus sp.
MPFWWRRRRKFWTNNRFNYTKRRRYRKRWPRRRRRRRPYRRPVRRRRRKLRKVKRKKK

SEQ ID NO: 927 - protein, 202 aa, Gammatorquevirus sp.
SLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYL

SEQ ID NO: 928 - protein, 79 aa, Gammatorquevirus sp.
NHGYYTITNWGAQSSTAYRPNSKVTDTTYYRYKNDRKNINIKSHEYEKSISYENGYFQSSFLQTQCIYTSERGEACIAE

SEQ ID NO: 929 - protein, 160 aa, Gammatorquevirus sp.
KPLGIAIYNPVKDNGDGNMIYLVSTLANTWDQPPKDSAILIQGVPIWLGLFGYLDYCRQIKADKTWLDSHVLVIQSPAIFTYPNPGAGKWYCPLSQSFINGNGPFNQPPTLLQKAKWFPQIQYQQEIINSFVESGPFVPKYANQTESNWELKYKYVFTFK

SEQ ID NO: 930 - protein, 163 aa, Gammatorquevirus sp.
WGGPQFHEPEIADPSKQEQYDVPDTFYQTIQIEDPEGQDPRSLIHDWDYRRGFIKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 931 - protein, 728 aa, Alphatorquevirus sp.; identical to SEQ ID NO: 900

SEQ ID NO: 932 - protein, 66 aa, Alphatorquevirus sp.
TAWWWGRWRRRWRRRRPYTTRLRRRRARRAFPRRRRRRFVSRRWRRPYRRRRRRGRRRRRRRRRHK

SEQ ID NO: 933 - protein, 211 aa, Alphatorquevirus sp.
PTLILRQWQPDCIRHCKITGWMPLIICGKGSTQFNYITHADDITPRGASYGGNFTNMTFSLEAIYEQFLYHRNRWSASNHDLELCRYKGTTLKLYRHPEVDYIVTYSRTGPFEISHMTYLSTHPMLMLLNKHHIVVPSLKTKPRGRKAIKVRIRPPKLMNNKWYFTRDFCNIGLFQLWATGLELRNPWLRMSTLSPCIGFNVLKNSIYTNL

SEQ ID NO: 934 - protein, 118 aa, Alphatorquevirus sp.
SNLPQYKNERLNIINNILHPQEITGTNNKKWQYTYTKLMAPIYYSANRASTYDWENYSKETNYNNTYVKFTQKRQEKLTKIRKEWQMLYPQQPTALPDSYDLLQEYGLYSPYYLNPTR

SEQ ID NO: 935 - protein, 166 aa, Alphatorquevirus sp.
INLDWMTPYTHVRYNPLVDKGFGNRIYIQWCSEADVSYNRTKSKCLLQDMPLFFMCYGYIDWAIKNTGVSSLVKDARICIRCPYTEPQLVGSTEDIGFVPISETFMRGDMPVLAPYIPLSWFCKWYPNIAHQKEVLESIISCSPFMPRDQDMNGWDITIGYKMDFL

SEQ ID NO: 936 - protein, 167 aa, Alphatorquevirus sp.
WGGSPLPSQPIDDPCQQGTHPIPDPDKHPRLLQVSNPKLLGPRTVFHKWDIRRGQFSKRSIKRVSEYSSDDESLAPGLPSKRNKLDSAFRGENREQKECYSLLKALEEEETPEEEEPAPQEKAQKEELLHQLQLQRRHQRVLRRGLKLVFTDILRLRQGVHWNPELT

SEQ ID NO: 937 - protein, 767 aa, Alphatorquevirus sp.; identical to SEQ ID NO: 908

SEQ ID NO: 938 - protein, 69 aa, Alphatorquevirus sp.
MAYGWWRRRRRRPWWRRRWRRWRRRRRPRRRRPRRRYRRRRTVRRRGRGRWTRAHRRWRRKGKRSRKKK

SEQ ID NO: 939 - protein, 200 aa, Alphatorquevirus sp.
IIIRQWQPNYTRRCNIVGYMPLLICGENTVATNYATHSDDSYYPGPFGGGMTTDKFTLRILYDEYKRFMNYWTSSNEDLDLCRYLGCTLYVFRHPEVDFIIIINTSPPFLDTEITGPSIHPGMMALNKRSRWIPSIKNRPGRKHYIKIKVGAPRMFTDKWYPQTDLCDMTLLTIFASAADMQYPFGSPLTDTIVVSFQVL

SEQ ID NO: 940 - protein, 155 aa, Alphatorquevirus sp.
QSMYNDCLSVLPDNFAETSGKGTQLHENIIQHLPYYNTTQTQAQFKRFIENMNATNGDNIWASYINTTKFSSANTPKNDTGIGGPYTTYSDSWYKGTVYNDKIKTIPIKASKLYYEQTKNLIGITFTGSTHRLHYCGGLYSSVWLSAGRSYFETK

SEQ ID NO: 941 - protein, 160 aa, Alphatorquevirus sp.
GPYTDITYNPFSDRGEGNMLWIDWLTKNDSVYSKTSSKCLIENLPLWASVYGYKEYCSKVTGDTNIEHNCRCVIRSPYTVPQLLDHNNPFRGYVPYSFNFGNGKMPGGSSLVPIRMRAKWYPTLFHQKEVLEAIAQAGPFAYHSDIKKVSLGIKYRFKWV

SEQ ID NO: 942 - protein, 183 aa, Alphatorquevirus sp.
WGGNPVSQQVVRNPCKTTQGSSGNRVPRSIQVVDPRYNTPELTIHAWDFRHGFFGRKAIKRMQEQPIPHDTFSAGFKRSRRDTEALQCSQEEQQKENLLFPVQQLKRVPPWETSQESQSEEENSQKQETLSQQLRDQLHKQRLMGEQLRSLLYQMQRVQQNQHINPMLLPKGLALTSISHNVI

SEQ ID NO: 943 - protein, 766 aa, Alphatorquevirus sp.; identical to SEQ ID NO: 916

SEQ ID NO: 944 - protein, 70 aa, Alphatorquevirus sp.
MAWRWWWQRRWRRRRWPRRRWRRLRRRRPRRPVRRRRRRTTVRRRRWRGRRGRRTYTRRAVRRRRRPRKR

SEQ ID NO: 945 - protein, 201 aa, Alphatorquevirus sp.
LVLTQWSPQTVRNCSIRGIVPMVICGHTKAGRNYAIHSEDFTTQIQPFGGSFSTTTWSLKVLWDEHQKFQNRWSYPNTQLDLARYRGVTFWFYRDQKTDYIVQWSRNPPFKLNKYSSAMYHPGMMMQAKRKLVVPSFQTRPKGKKRYRVTIKPPNMFADKWYTQEDLCPVPLVQIVVSAASLLHPFCPPQTNNPCITFQVL

SEQ ID NO: 946 - protein, 147 aa, Alphatorquevirus sp.
KDIYDECIGVNETMKDKYKKLQTTLYTTCTYYQTTQVLAQLSPAFQPAMKPTTTQSAATATTLGNYVPELKYNNGSFHTGQNAVFGMCSYKPTDSIMTKANGWFWQNLMVDNNLHSSYGKATLECMEYHTGIYSSIFLSPQRSLEFP

SEQ ID NO: 947 - protein, 161 aa, Alphatorquevirus sp.
AAYQDVTYNPNCDRAVGNVVWFQYSTKMDTNFDETKCKCVLKNIPLWAAFNGYSDFIMQELSISTEIHNFGIVCFQCPYTFPPCFNKNKPLKGYVFYDTTFGNGKMPDGSGHVPIYWQQRWWIRLAFQVQVMHDFVLTGPFSYKDDLANTTLTARYKFKFK

SEQ ID NO: 948 - protein, 187 aa, Alphatorquevirus sp.
WGGNIIPEQIIKNPCHREQSLASYPDRQRRDLQVVDPSTMGPIYTFHTWDWRRGLFGADAIQRVSQKPGDALRFTNPFKRPRYLPPTDREDYRQEEDFALQEKRRRTSTEEAQDEESPPESAPLLQQQQQQRQLSVHLAEQQRLGVQLRYILQEVLKTQAGLHLNPLLLGPPQTRSISLSPPKAYSP

SEQ ID NO: 949 - protein, 21 aa, synthetic construct
  VARIANT 1: note = W or F
  VARIANT 2: note = Any amino acid
  VARIANT 3: note = Any amino acid
  VARIANT 4: note = Any amino acid
  VARIANT 5: note = Any amino acid
  VARIANT 6: note = Any amino acid
  VARIANT 7: note = Any amino acid
  VARIANT 8: note = Any amino acid
  VARIANT 10: note = Any amino acid
  VARIANT 11: note = Any amino acid
  VARIANT 12: note = Any amino acid
  VARIANT 14: note = Any amino acid
  VARIANT 16: note = Any amino acid
  VARIANT 17: note = Any amino acid
  VARIANT 18: note = Any amino acid
  VARIANT 19: note = Any amino acid
  VARIANT 20: note = Any amino acid
XXXXXXXXHXXXCXCXXXXXH

SEQ ID NO: 950-1000 - skipped (51 entries)

SEQ ID NO: 1001 - DNA, 2845 bp, Betatorquevirus sp.
  source 1..2845: mol_type = genomic DNA
ttattaatattcaacaggaaaaccacctaatttaaattgccgaccacaaaccgtcactaacttccttatttaacattacttcccttttaaccaatgaatattcatacaacacatcacacttcctgggaggagacataaaactatataactaactacacagacgaatggctgagtttatgccgctagacggaggacgcacagctactgctgcgacctgaacttgggcgggtgccgaaggtgagtgtaaccaccgtagtcaaggggcaattcgggctagttcagtctagcggaacgggcaagattattaatacaaacttatttttacagatgagcaaacaactaaaaccaactttatacaaagacaaatcattggaattacaatggctaaacaacatttttagctctcacgacctgtgctgcggctgcaacgatccagttttacatttactgattttaattaacaaaaccggagaagcacctaaaccagaagaagacattaaaaatataaaatgcctccttactggcgccaaaaatactaccgaagaagatatagacctttctcctggagaactagaagaattattcaaagaagaaaaagatggagataccgcaaaccaagaaaaacatactggagaagaaaactgcgggtaagaaaacgtttttataaaagaaagttaaaaaaaattgtacttaaacagtttcaaccaaaaattattagaagatgtacaatatttggaacaatctgcctatttcaaggctctccagaaagagccaacaataattatattcaaacaatctactcctacgtaccagataaagaaccaggaggagggggatggactttaataactgaaagcttaagtagtttatgggaagactgggaacatttaaaaaatgtatggactcaaagtaacgctggtttaccacttgtaagatacgggggagtaacattatacttttatcaatctgcctatactgactatattgctcaagttttcaactgttatcctatgacagacacaaaatacacacatgcagactcagcaccaaacagaatgttattaaaaaaacatgtaataagagtacctagcagagaaacacgcaaaaaaagaaagccatacaaaagagttagagtaggacctccttctcaaatgcaaaacaaatggtactttcaaagagacatatgtgaaataccattaataatgattgcagccacagccgttgactttagatatcccttttgtgcaagcgactgtgctagtaacaacttaactctaacatgtttaaacccactattgtttcaaaaccaagactttgaccacccatccgatacacaaggctactttccaaaacctggagtatatctatactcaacacaaagaagtaacaagccaagttcttcagactgtatatacttaggaaacacaaaagacaatcaagaaggtaaatctgcaagtagtctaatgactctaaaaacacaaaaaataacagattggggaaatccattttggcattattatatagacggttctaaaaaaatattttcttactttaaacccccatcacaattagacagcagcgactttgaacacatgacagaattagcagaaccaatgtttatacaagttagatacaacccagaaagagacacaggacaaggaaacttaatatacgtaacagaaaactttagaggacaacactgggaccctccatctagtgacaacctaaaattagatggatttcccttatatgacatgtgctggggtttcatagactggatagaaaaagttcatgaaacagaaaacttacttaccaactactgcttctgtattagaagcagcgctttcaatgaaaaaaaaacagtttttatacctgtagatcattcatttttaacaggttttagcccatatgaaactccagttaaatcatcagaccaagctcactggcacccacaaataagatttcaaacaaaatcaataaatgacatttgtttaacaggccccggttgtgctaggtccccatatggcaattacatgcaggcaaaaatgagttataaatttcatgtaaaatggggaggatgtccaaaaacttatgaaaaaccatatgatccttgttcacagcccaattggactattccccataacctcaatgaaacaatacaaatccagaatccaaacacatgcccacaaacagaactccaagaatgggactggcgacgtgatattgttacaaaaaaagctatcgaaagaattagacaacacacggaacctcatgaaactttgcaaatctctacaggttccaaacacaacccaccagtacacagacaaacatcaccgtggacggactcagaaacggactcggaagaggaaaaagaccaaacacaagagatccagatccagctcaacaagctcagaaagcatcaacagcatctcaagcagcagctcaagcagtacctgaaaccccaaaatatagaatagttgcaagcaacataaaagttgaactttttcctactaaaaaaccttttaaaaacagacgctttactccttctgaaagagaaacagaaagacaatgtgctaaagctttttgtagaccagaaagacatttcttttatgatcctcctttttacccttactgtgtacctgaacctattgtaaactttgctttgggatataaaatttaaggccaacaaatttcacttagtggtgtctgtttattaaagtttaaccttaataagcatactccgcctccctacattaaggcgccaaaagggggctccgcccccttaaaccccaagggggctccgcccccttaaacccccaagggggctccgcccccttacaccccc

SEQ ID NO: 1002 - protein, 107 aa, Betatorquevirus sp.
MSKQLKPTLYKDKSLELQWLNNIFSSHDLCCGCNDPVLHLLILINKTGEAPKPEEDIKNIKCLLTGAKNTTEEDIDLSPGELEELFKEEKDGDTANQEKHTGEENCG

SEQ ID NO: 1003 - protein, 218 aa, Betatorquevirus sp.
MSKQLKPTLYKDKSLELQWLNNIFSSHDLCCGCNDPVLHLLILINKTGEAPKPEEDIKNIKCLLTGAKNTTEEDIDLSPGELEELFKEEKDGDTANQEKHTGEENCGPIGLFPITSMKQYKSRIQTHAHKQNSKNGTGDVILLQKKLSKELDNTRNLMKLCKSLQVPNTTHQYTDKHHRGRTQKRTRKRKKTKHKRSRSSSTSSESINSISSSSSSST

SEQ ID NO: 1004 - protein, 234 aa, Betatorquevirus sp.
MSKQLKPTLYKDKSLELQWLNNIFSSHDLCCGCNDPVLHLLILINKTGEAPKPEEDIKNIKCLLTGAKNTTEEDIDLSPGELEELFKEEKDGDTANQEKHTGEENCGFQTQPTSTQTNITVDGLRNGLGRGKRPNTRDPDPAQQAQKASTASQAAAQAVPETPKYRIVASNIKVELFPTKKPFKNRRFTPSERETERQCAKAFCRPERHFFYDPPFYPYCVPEPIVNFALGYKI

SEQ ID NO: 1005 - protein, 655 aa, Betatorquevirus sp.
MPPYWRQKYYRRRYRPFSWRTRRIIQRRKRWRYRKPRKTYWRRKLRVRKRFYKRKLKKIVLKQFQPKIIRRCTIFGTICLFQGSPERANNNYIQTIYSYVPDKEPGGGGWTLITESLSSLWEDWEHLKNVWTQSNAGLPLVRYGGVTLYFYQSAYTDYIAQVFNCYPMTDTKYTHADSAPNRMLLKKHVIRVPSRETRKKRKPYKRVRVGPPSQMQNKWYFQRDICEIPLIMIAATAVDFRYPFCASDCASNNLTLTCLNPLLFQNQDFDHPSDTQGYFPKPGVYLYSTQRSNKPSSSDCIYLGNTKDNQEGKSASSLMTLKTQKITDWGNPFWHYYIDGSKKIFSYFKPPSQLDSSDFEHMTELAEPMFIQVRYNPERDTGQGNLIYVTENFRGQHWDPPSSDNLKLDGFPLYDMCWGFIDWIEKVHETENLLTNYCFCIRSSAFNEKKTVFIPVDHSFLTGFSPYETPVKSSDQAHWHPQIRFQTKSINDICLTGPGCARSPYGNYMQAKMSYKFHVKWGGCPKTYEKPYDPCSQPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 1006 - protein, 164 aa, Betatorquevirus sp.
MPPYWRQKYYRRRYRPFSWRTRRIIQRRKRWRYRKPRKTYWRRKLRPNWTIPHNLNETIQIQNPNTCPQTELQEWDWRRDIVTKKAIERIRQHTEPHETLQISTGSKHNPPVHRQTSPWTDSETDSEEEKDQTQEIQIQLNKLRKHQQHLKQQLKQYLKPQNIE

SEQ ID NO: 1007 - protein, 99 aa, Betatorquevirus sp.
MPPYWRQKYYRRRYRPFSWRTRRIIQRRKRWRYRKPRKTYWRRKLRVPNTTHQYTDKHHRGRTQKRTRKRKKTKHKRSRSSSTSSESINSISSSSSSST

SEQ ID NO: 1008 - DNA, 2912 bp, Betatorquevirus sp.
  source 1..2912: mol_type = genomic DNA
taataaatattcaacaggaaaaccacctaatttaaattgccgaccacaaaccgtcacttagttcctctttttccacaacttcctcttttactaatgaatattcatgtaattaattaataatcaccgtaattccggggaggagcctttaaactataaaactaactacacattcgaatggctgagtttatgccgccagacggagacgggatcacttcagtgactccaggctgatcaagggcgggtgccgaaggtgagtgaaaccaccgtagtcaaggggcaattcgggctagatcagtctggcggaacgggcaagaaacttaaaatgtactttattttacagaaatgttcaaatctccaacatacttaacaactaaaggcaaaaacaatgccttaatcaactgcttcgttggagaccacgatcttctgtgcagctgtaacaatcctgcctaccattgcctccaaatacttgcaactaccttagcacctcaactaaaacaagaagaaaaacaacaaataatacaatgccttggtggtacagacgccgtagctacaacccgtggagacgaagaaattggtttagaagacctagaaaaactatttacagaagatacagaagaagacgccgctgggtaagaagaaaacctttttacaaacgtaaaattaagagactaaatatagtagaatggcaacctaaatcaattagaaaatgtagaataaaaggaatgctatgcttgtttcaaacgacagaagacagactgtcatataactttgatatgtatgaagagtctattataccagaaaaactgccgggaggggggggatttagcattaagaatataagcttatatgccttataccaagaacacatacatgcacacaacatatttacacacacaaacacagacagaccactagcaagatacacaggctgttctttaaaattctaccaaagcaaagacatagactacgtagtaacatattctacatcactcccactaagaagctcaatgggaatgtacaactccatgcaaccatccatacatctaatgcaacaaaacaaactaattgtaccaagcaaacaaacacaaaaaagaagaaaaccatatattaaaaaacatatatcaccaccaacacaaatgaaatctcaatggtactttcaacataacattgcaaacataccgctactaatgataagaaccacagcattaacattagataattactatataggaagcagacaattaagtacaaatgtcactatacatacacttaacacaacatacatccaaaacagagactggggagacagaaataaaacttactactgccaaacattaggaacacaaagatacttcctatatggaacacattcaactgcacaaaatattaatgacataaagctacaagaactaatacctttaacaaacacacaagactatgtacaaggctttgattggacagaaaaagacaaacataacataacaacctacaaagaattcttaactaaaggagcaggaaatccatttcacgcagaatggataacagcacaaaacccagtaatacacacagcaaacagtcctacacaaatagaacaaatatacaccgcttcaacaacaacattccaaaacaaaaaactaacagacctaccaacgccaggatatatatttataactccaacagtaagcttaagatacaacccatacaaagacctagcagaaagaaacaaatgctactttgtaagaagcaaaataaatgcacacgggtgggacccagaacaacaccaagaattaataaacagtgacctaccacaatggttactattatttggctacccagactacataaaaagaacacaaaactttgcattagtagacacaaattacatactagtagaccactgcccatacacaaatccagaaaaaacaccatttatacctttaagcacatcatttatagaaggtagaagcccatacagtccttcagacacacatgaaccagatgaagaagaccaaaacaggtggtacccatgctaccaatatcaacaagaatcaataaattcaatatgtcttagcggtccaggcacaccaaaaataccaaaaggaataacagcagaagcaaaagtaaaatattcctttaattttaagtggggtggtgacctaccaccaatgtctacaattacaaacccgacagaccagccaacatatgttgttcccaataacttcaatgaaacaacttcgttacagaatccaaccaccagaccagagcacttcttgtactcctttgacgaaaggaggggacaacttacagaaaaagctacaaaacgcttgcttaaagactgggaaactaaagaaacttctttattgtctacagaatacagattcgcggagccaacacaaacacaagccccacaagaggacccgtcctcggaagaagaagaagagagcaacctcttcgagcgactcctccgacagcgaaccaagcagctccagctcaagcgcagaataatacaaacattgaaagacctacaaaaattagaataactaacagcaaaaacaccgtttacctatttccacctgaacaaaagaacagaagactaacaccatgggaaatacaagaagacaaagaaatagccaatttatttggcagaccacatagatactttttaaaagacattcctttctattgggatatacccccagagcctaaagtaaactttgatttaaattttcaataaagaaataaagggcaaggccccattaactcaaagtcggtgtctacctctttaagtttaactttactaaacggactccgcctccctaaatttgggcgccaaaagggggctccgcccccttaaaccccagggggctccgccccctaaaacccccaagggggctacgcccccttacaccccc

SEQ ID NO: 1009 - protein, 96 aa, Betatorquevirus sp.
MFKSPTYLTTKGKNNALINCFVGDHDLLCSCNNPAYHCLQILATTLAPQLKQEEKQQIIQCLGGTDAVATTRGDEEIGLEDLEKLFTEDTEEDAAG

SEQ ID NO: 1010 - protein, 198 aa, Betatorquevirus sp.
MFKSPTYLTTKGKNNALINCFVGDHDLLCSCNNPAYHCLQILATTLAPQLKQEEKQQIIQCLGGTDAVATTRGDEEIGLEDLEKLFTEDTEEDAAGQHMLFPITSMKQLRYRIQPPDQSTSCTPLTKGGDNLQKKLQNACLKTGKLKKLLYCLQNTDSRSQHKHKPHKRTRPRKKKKRATSSSDSSDSEPSSSSSSAE

SEQ ID NO: 1011 - protein, 216 aa, Betatorquevirus sp.
MFKSPTYLTTKGKNNALINCFVGDHDLLCSCNNPAYHCLQILATTLAPQLKQEEKQQIIQCLGGTDAVATTRGDEEIGLEDLEKLFTEDTEEDAAGIQIRGANTNTSPTRGPVLGRRRREQPLRATPPTANQAAPAQAQNNTNIERPTKIRITNSKNTVYLFPPEQKNRRLTPWEIQEDKEIANLFGRPHRYFLKDIPFYWDIPPEPKVNFDLNFQ

SEQ ID NO: 1012 - protein, 672 aa, Betatorquevirus sp.
MPWWYRRRSYNPWRRRNWFRRPRKTIYRRYRRRRRWVRRKPFYKRKIKRLNIVEWQPKSIRKCRIKGMLCLFQTTEDRLSYNFDMYEESIIPEKLPGGGGFSIKNISLYALYQEHIHAHNIFTHTNTDRPLARYTGCSLKFYQSKDIDYVVTYSTSLPLRSSMGMYNSMQPSIHLMQQNKLIVPSKQTQKRRKPYIKKHISPPTQMKSQWYFQHNIANIPLLMIRTTALTLDNYYIGSRQLSTNVTIHTLNTTYIQNRDWGDRNKTYYCQTLGTQRYFLYGTHSTAQNINDIKLQELIPLTNTQDYVQGFDWTEKDKHNITTYKEFLTKGAGNPFHAEWITAQNPVIHTANSPTQIEQIYTASTTTFQNKKLTDLPTPGYIFITPTVSLRYNPYKDLAERNKCYFVRSKINAHGWDPEQHQELINSDLPQWLLLFGYPDYIKRTQNFALVDTNYILVDHCPYTNPEKTPFIPLSTSFIEGRSPYSPSDTHEPDEEDQNRWYPCYQYQQESINSICLSGPGTPKIPKGITAEAKVKYSFNFKWGGDLPPMSTITNPTDQPTYVVPNNFNETTSLQNPTTRPEHFLYSFDERRGQLTEKATKRLLKDWETKETSLLSTEYRFAEPTQTQAPQEDPSSEEEEESNLFERLLRQRTKQLQLKRRIIQTLKDLQKLE

SEQ ID NO: 1013 - protein, 80 aa, Betatorquevirus sp.
MPWWYRRRSYNPWRRRNWFRRPRKTIYRRYRRRRRWNTDSRSQHKHKPHKRTRPRKKKKRATSSSDSSDSEPSSSSSSAE

SEQ ID NO: 1014 - DNA, 3853 bp, Alphatorquevirus sp.
  source 1..3853: mol_type = genomic DNA
ggcttagtgcgtcaccacccacgtgacccgcctccgccaattaacaggtacttcgtacacttcctgggcgggcttataagactaatataagtagctgcacttccgaatggctgagttttccacgcccgtccgcagcggtgaagccacggagggagctcagcgcgtcccgagggcgggtgccggaggtgagtttacacaccgcagtcaaggggcaattcgggctcgggactggccgggctttgggcaaggctcttaaaaaagctatgtttattggcaggcactaccgaaagaaaagggcgctgctactgctatctgtgcattctacaaagacaaaagggaaacttctaatagctatgtggactcccccacgcaatgatcaacaataccttaactggcaatggtacacttctgtacttagctcccactctgctatgtgcgggtgttccgacgctatcgctcatcttaatcatcttgctaatctgcttcgtgccccgcaaaatccgcccccgcctgataatccaagacccctacccgtgcgagcactgcctgctcccccggctgcccacgaggcagccggtgatcgagcaccatggcctatgggtggtggaggagacgccggaggcgctggcgcaggtggagacgccgaccatggaggcgccgctggaggacccgcagacgcagacctgctagacgccgtggccgccgcagaaacgtaaggagacggcgcagagggaggtggagaaggaggtacaggaggtggaaaagaaagggcagacgtagaagaaaagcaaaaataataataagacagtggcagccaaactacagaagaagatgtaatatagtgggctacctccctatacttatctgtggtggaaatactgtttctagaaactatgccacacactcagacgatactaactatccaggaccctttgggggaggcatgaccacagacaaattcagccttagaatactatatgatgaatacaaaagatttatgaactactggacagcctcaaatgaggacctagatctctgtagatatctaggatgcactttttacttctttagacaccctgaagtagactttattataaaaataaacaccatgcccccattcttagatacaaccataacagcacctagcatacacccaggcctcatggccctagacaaaagagccagatggattccttctcttaaaaatagaccaggtaaaaaacactatataaaaattagagtaggggctcctaaaatgttcacagataaatggtaccctcaaacagacctctgtgacatgacactgctaactatctatgcaaccgcagcggatatgcaatatccgttcggctcaccactaactgacactgtggttgttaactcccaagttctgcaatccatgtatgatgaaacaattagcatattacctgatgaaaaaactaaaagaaatagccttcttacttctataagaagctacatacctttttataatactacacaaacaatagctcaattaaaaccatttgtagatgcaggaggacacacaacaggctcaacaacaactacatggggacaactattaaacacaactaaatttaccactaccacaacaaccacatacacataccctggcaccacaaatacagcagtaacatttataacagccaatgatacctggtacaggggaacagcatataaagataacattaaagatgtaccacaaaaagcagcacaattatactttcaaacaacacaaaaactactaggaaacacattccatggctcagatgaaacacttgaataccatgcaggcctatacagctctatctggctatcaccaggtagatcctactttgaaacaccaggtgcatacacagacattaaatataacccttttacagacagaggagaaggcaacatgctgtggatagactggctaagtaaaaaaaacatgaaatatgacaaagtgcaaagtaagtgcctagtagcagacctaccactgtgggcagcagcatatggttatgtagaattctgctctaaaagcacaggagacacaaacatacacatgaatgccagactactaataagaagtccttttacagacccccagctaatagtacacacagaccccactaaaggctttgtaccctattctttaaactttggaaatggtaaaatgccaggaggtagcagcaatgttcccataagaatgagagctaagtggtaccccactttatcccaccaacaagaagttctagaggccttagcacagtcaggaccctttgcttatcactcagacattaaaaaagtatctctaggcataaaataccgttttaagtggatctggggtggaaaccccgttcgccaacaggttgttagaaatccctgcaaggaaccccactcctcgggcaatagagtccctagaagcatacaaatcgttgacccgagatacaactcaccggaacttaccatccatgcctgggacttcagacgtggcttctttggcccgaaagctattcaaagaatgcaacaacaaccaactgctactgaatttttttcagcaggccgcaagagacccagaagggacacagaagtgtatcagtccgaccaagaaaaggagcaaaaagaaagctcgcttttccccccagtcaagctcctccgaagagtccccccgtgggaggactcggaacaggagcaaagcgggtcgcaaagctcagaggaagagacggcgaccctctcccagcagctcaaacagcagctgcagcagcagcgagtcttgggagtcaaactcagactcctgttcaaccaagtccaaaaaatccaacaaaatcaagatatcaaccctaccttgttaccaagggggggggatctagtatccttctttcaggctgtaccataaatatgtttccagaccctaaaccttactgcccctccagcaatgactggaaagaagagtatgaggcctgtaaatattgggatagacctcccagacacaaccttagagacccccccttttacccctgggcccctaaaaacaatccttgcaatgtaagctttaaacttggcttcaaataaactaggccgtgggagtttcacttgtcggtgtctacctctataagtcactaagcactccgagcgcagcgaggagtgcgacccttccccctggtgcaacgccctcggcggccgcgcgctacgccttcggctgcgcgcggcacctcggacccccgctcgtgctgacacgcttgcgcgtgtcagaccacttcgggctcgcgggggtcgggaaatttgctaaacagactccgagttgccattggacactgtagctatgaatcagtaacgaaagtgagtggggccagacttcgccataaggcctttatcttcttgccatttgtcagtattgggggtcgccataaactttgggctccattttaggccttccggactacaaaaatcgccatatttgtgacgtcagagccgccattttaagtcagctctggggaggcgtgacttccagttcaaaggtcatcctcaccataactggcacaaaatggccgccaacttcttccgggtcaaaggtcactgctacgtcataggtgacgtggggggggacctacttaaacacggaagtaggccccgacacgtcactgtcacgtgacagtacgtcacagccgccattttgttttacaaaatagccgacttccttcctcttttttaaaaaaaggcgccaaaaaaccgtcggcgggggggccgcgcgctgcgcgcgcggcccccgggggaggcacagcctcccccccccgcgcgcatgcgcgcgggtccccccccctccggggggctccgccccccggcccccccc

SEQ ID NO: 1015 - protein, 120 aa, Alphatorquevirus sp.
MWTPPRNDQQYLNWQWYTSVLSSHSAMCGCSDAIAHLNHLANLLRAPQNPPPPDNPRPLPVRALPAPPAAHEAAGDRAPWPMGGGGDAGGAGAGGDADHGGAAGGPADADLLDAVAAAET

SEQ ID NO: 1016 - protein, 286 aa, Alphatorquevirus sp.
MWTPPRNDQQYLNWQWYTSVLSSHSAMCGCSDAIAHLNHLANLLRAPQNPPPPDNPRPLPVRALPAPPAAHEAAGDRAPWPMGGGGDAGGAGAGGDADHGGAAGGPADADLLDAVAAAETLLEIPARNPTPRAIESLEAYKSLTRDTTHRNLPSMPGTSDVASLARKLFKECNNNQLLLNFFQQAARDPEGTQKCISPTKKRSKKKARFSPQSSSSEESPRGRTRNRSKAGRKAQRKRRRPSPSSSNSSCSSSESWESNSDSCSTKSKKSNKIKISTLPCYQGGGI

SEQ ID NO: 1017 - protein, 289 aa, Alphatorquevirus sp.
MWTPPRNDQQYLNWQWYTSVLSSHSAMCGCSDAIAHLNHLANLLRAPQNPPPPDNPRPLPVRALPAPPAAHEAAGDRAPWPMGGGGDAGGAGAGGDADHGGAAGGPADADLLDAVAAAETPQETQKGHRSVSVRPRKGAKRKLAFPPSQAPPKSPPVGGLGTGAKRVAKLRGRDGDPLPAAQTAAAAAASLGSQTQTPVQPSPKNPTKSRYQPYLVTKGGGSSILLSGCTINMFPDPKPYCPSSNDWKEEYEACKYWDRPPRHNLRDPPFYPWAPKNNPCNVSFKLGFK

SEQ ID NO: 1018 - protein, 185 aa, Alphatorquevirus sp.
MWTPPRNDQQYLNWQWPQETQKGHRSVSVRPRKGAKRKLAFPPSQAPPKSPPVGGLGTGAKRVAKLRGRDGDPLPAAQTAAAAAASLGSQTQTPVQPSPKNPTKSRYQPYLVTKGGGSSILLSGCTINMFPDPKPYCPSSNDWKEEYEACKYWDRPPRHNLRDPPFYPWAPKNNPCNVSFKLGFK

SEQ ID NO: 1019 - protein, 105 aa, Alphatorquevirus sp.
MINNTLTGNGTLLYLAPTLLCAGVPTLSLILIILLICFVPRKIRPRLIIQDPYPCEHCLLPRLPTRQPVIEHHGLWVVEETPEALAQVETPTMEAPLEDPQTQTC

SEQ ID NO: 1020 - protein, 769 aa, Alphatorquevirus sp.
MAYGWWRRRRRRWRRWRRRPWRRRWRTRRRRPARRRGRRRNVRRRRRGRWRRRYRRWKRKGRRRRKAKIIIRQWQPNYRRRCNIVGYLPILICGGNTVSRNYATHSDDTNYPGPFGGGMTTDKFSLRILYDEYKRFMNYWTASNEDLDLCRYLGCTFYFFRHPEVDFIIKINTMPPFLDTTITAPSIHPGLMALDKRARWIPSLKNRPGKKHYIKIRVGAPKMFTDKWYPQTDLCDMTLLTIYATAADMQYPFGSPLTDTVVVNSQVLQSMYDETISILPDEKTKRNSLLTSIRSYIPFYNTTQTIAQLKPFVDAGGHTTGSTTTTWGQLLNTTKFTTTTTTTYTYPGTTNTAVTFITANDTWYRGTAYKDNIKDVPQKAAQLYFQTTQKLLGNTFHGSDETLEYHAGLYSSIWLSPGRSYFETPGAYTDIKYNPFTDRGEGNMLWIDWLSKKNMKYDKVQSKCLVADLPLWAAAYGYVEFCSKSTGDTNIHMNARLLIRSPFTDPQLIVHTDPTKGFVPYSLNFGNGKMPGGSSNVPIRMRAKWYPTLSHQQEVLEALAQSGPFAYHSDIKKVSLGIKYRFKWIWGGNPVRQQVVRNPCKEPHSSGNRVPRSIQIVDPRYNSPELTIHAWDFRRGFFGPKAIQRMQQQPTATEFFSAGRKRPRRDTEVYQSDQEKEQKESSLFPPVKLLRRVPPWEDSEQEQSGSQSSEEETATLSQQLKQQLQQQRVLGVKLRLLFNQVQKIQQNQDINPTLLPRGGDLVSFFQAVP

SEQ ID NO: 1021 - protein, 216 aa, Alphatorquevirus sp.
MAYGWWRRRRRRWRRWRRRPWRRRWRTRRRRPARRRGRRRNVVRNPCKEPHSSGNRVPRSIQIVDPRYNSPELTIHAWDFRRGFFGPKAIQRMQQQPTATEFFSAGRKRPRRDTEVYQSDQEKEQKESSLFPPVKLLRRVPPWEDSEQEQSGSQSSEEETATLSQQLKQQLQQQRVLGVKLRLLFNQVQKIQQNQDINPTLLPRGGDLVSFFQAVP

SEQ ID NO: 1022 - protein, 143 aa, Alphatorquevirus sp.
MAYGWWRRRRRRWRRWRRRPWRRRWRTRRRRPARRRGRRRNAARDPEGTQKCISPTKKRSKKKARFSPQSSSSEESPRGRTRNRSKAGRKAQRKRRRPSPSSSNSSCSSSESWESNSDSCSTKSKKSNKIKISTLPCYQGGGI

SEQ ID NO: 1023 - protein, 228 aa, Betatorquevirus sp.
MPWWYRRRSYNPWRRRNWFRRPRKTIYRRYRRRRRWVRRKPFYKRKIKRLNIVEWQPKSIRKCRIKGMLCLFQTTEDRLSYNFDMYEESIIPEKLPGGGGFSIKNISLYALYQEHIHAHNIFTHTNTDRPLARYTGCSLKFYQSKDIDYVVTYSTSLPLRSSMGMYNSMQPSIHLMQQNKLIVPSKQTQKRRKPYIKKHISPPTQMKSQWYFQHNIANIPLLMIRTTA

SEQ ID NO: 1024 - protein, 222 aa, Betatorquevirus sp.
MPYYYRRRRYNYRRPRWYGRGWIRRPFRRRFRRKRRVRPTYTTIPLKQWQPPYKRTCYIKGQDCLIYYSNLRLGMNSTMYEKSIVPVHWPGGGSFSVSMLTLDALYDIHKLCRNWWTSTNQDLPLVRYKGCKITFYQSTFTDYIVRIHTELPANSNKLTYPNTHPLMMMMSKYKHIIPSRQTRRKKKPYTKIFVKPPPQFENKWYFATDLYKIPLLQIHCTA

SEQ ID NO: 1025 - protein, 238 aa, Gammatorquevirus sp.
MPFWWRRRRKFWTNNRFNYTKRRRYRKRWPRRRRRRRPYRRPIRRRRRKLRKVKRKKKSLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASA

SEQ ID NO: 1026 - protein, 246 aa, Alphatorquevirus sp.
MAYGWWRRRRRRWRRWRRRPWRRRWRTRRRRPARRRGRRRNVRRRRRGRWRRRYRRWKRKGRRRRKAKIIIRQWQPNYRRRCNIVGYLPILICGGNTVSRNYATHSDDTNYPGPFGGGMTTDKFSLRILYDEYKRFMNYWTASNEDLDLCRYLGCTFYFFRHPEVDFIIKINTMPPFLDTTITAPSIHPGLMALDKRARWIPSLKNRPGKKHYIKIRVGAPKMFTDKWYPQTDLCDMTLLTIYATA

SEQ ID NO: 1027 - protein, 142 aa, Betatorquevirus sp.
EAKVKYSFNFKWGGDLPPMSTITNPTDQPTYVVPNNFNETTSLQNPTTRPEHFLYSFDERRGQLTEKATKRLLKDWETKETSLLSTEYRFAEPTQTQAPQEDPSSEEEEESNLFERLLRQRTKQLQLKRRIIQTLKDLQKLE

SEQ ID NO: 1028 - protein, 140 aa, Betatorquevirus sp.
NISMFYKFYFKWGGSPPKAINVENPAHQIQYPIPRNEHETTSLQSPGEAPESILYSFDYRHGNYTTTALSRISQDWALKDTVSKITEPDRQQLLKQALECLQISEETQEKKEKEVQQLISNLRQQQQLYRERIISLLKDQ

SEQ ID NO: 1029 - protein, 176 aa, Gammatorquevirus sp.
NWELKYKYIFTFKWGGPQFHEPEIADPSKQEQYDVPDTFYQTIQIEDPEGQDPRSLIHDWDYRRGFVKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 1030 - protein, 195 aa, Alphatorquevirus sp.
SLGIKYRFKWIWGGNPVRQQVVRNPCKEPHSSGNRVPRSIQIVDPRYNSPELTIHAWDFRRGFFGPKAIQRMQQQPTATEFFSAGRKRPRRDTEVYQSDQEKEQKESSLFPPVKLLRRVPPWEDSEQEQSGSQSSEEETATLSQQLKQQLQQQRVLGVKLRLLFNQVQKIQQNQDINPTLLPRGGDLVSFFQAVP

SEQ ID NO: 1031 - protein, 301 aa, Betatorquevirus sp.
TLDNYYIGSRQLSTNVTIHTLNTTYIQNRDWGDRNKTYYCQTLGTQRYFLYGTHSTAQNINDIKLQELIPLTNTQDYVQGFDWTEKDKHNITTYKEFLTKGAGNPFHAEWITAQNPVIHTANSPTQIEQIYTASTTTFQNKKLTDLPTPGYIFITPTVSLRYNPYKDLAERNKCYFVRSKINAHGWDPEQHQELINSDLPQWLLLFGYPDYIKRTQNFALVDTNYILVDHCPYTNPEKTPFIPLSTSFIEGRSPYSPSDTHEPDEEDQNRWYPCYQYQQESINSICLSGPGTPKIPKGITA

SEQ ID NO: 1032 - protein, 303 aa, Betatorquevirus sp.
NLQNPFVKPDKLSNNVTLWSLNTISIQNRNMSVDQGQSWPFKILGTQSFYFYFYTGANLPGDTTQIPVADLLPLTNPRINRPGQSLNEAKITDHITFTEYKNKFTNYWGNPFNKHIQEHLDMILYSLKSPEAIKNEWTIENMKWNQLNNAGTMALTPFNEPIFTQIQYNPDRDTGEDTQLYLLSNATGTGWDPPGIPELILEGFPLWLIYWGFADFQKNLKKVTNIDTNYMLVAKTKFTQKPGTFYLVILNDTFVEGNSPYEKQPLPEDNIKWYPQVQYQLEAQNKLLQTGPFTPNIQGQLSD

SEQ ID NO: 1033 - protein, 247 aa, Gammatorquevirus sp.
DLRHSYLGCCNENPQVFFYYLNHGYYIQTNWGAQSTTAYRPNSSVTETTYYKYKNEKKNINIKSSEYSESISYDKGYFQSSFLQTQCIYTSAAGDTCAAEKPLGIAIYNPVKDNGDGNMIYLVSTLANTWDQPPKDSAILIQGVPIWLGLFGYLDYCRQIKADKTWLDSHILVMQSPAIFTYPNPGAGKWYCPLSQSFINGNGPFNQPPTLLQKAKWFPQIQYQQEIINSFVESGPFVPKYANQTES

SEQ ID NO: 1034 - protein, 327 aa, Alphatorquevirus sp.
DMQYPFGSPLTDTVVVNSQVLQSMYDETISILPDEKTKRNSLLTSIRSYIPFYNTTQTIAQLKPFVDAGGHTTGSTTTTWGQLLNTTKFTTTTTTTYTYPGTTNTAVTFITANDTWYRGTAYKDNIKDVPQKAAQLYFQTTQKLLGNTFHGSDETLEYHAGLYSSIWLSPGRSYFETPGAYTDIKYNPFTDRGEGNMLWIDWLSKKNMKYDKVQSKCLVADLPLWAAAYGYVEFCSKSTGDTNIHMNARLLIRSPFTDPQLIVHTDPTKGFVPYSLNFGNGKMPGGSSNVPIRMRAKWYPTLSHQQEVLEALAQSGPFAYHSDIKKV

SEQ ID NO: 1035 - protein, 664 aa, Betatorquevirus sp.
MPPFRRNWWSNYYKPKWRRRRFPARRRRFTTTFRKRRRRTKKVKRHFYSKKYKRKLKKIILRQWQPSTIKRCKIEGYLCLFQAGKGRFSFNYASYKESFCPEHEPGGGGWGLQQLSLSNLYSQNCDLMNWWTKSNNHLNLCRYMGCEITLFRQDTVDYVFTYFDEQPKNVSKYYYATFHPTKLMLYNRKIVVPSFASQPHKRKPYKRKFIRAPKLMKNQWYFQQQLAPYPLLQFAATAVSLNKMFGSSKALNNNCTVYCLNTDFFRHPHFQYSAQPFTPDGGNQPTYIYTIQRGEPPITAYKYFNLTFLGQTMTNDPGIPLSNNNNYTKNEWGNVFFYKYLNNDELTYVSKETIDQVKQKKNQQITSGFTLKDQPYYYTLKYNPYKDKGKGNQAYWVSNIEATKQNWEPPNDPDLIISDFPFWIMLWGWEDMTKRIGKCRNLDNDWILVLRTQYFSDKMTAYVPLSEAFIHGQGPYFVDRSEIKGQDYAHWYPRYRYQREAIENILMTGPMVCRADNINNIQSLIKYKFFFKWGGTPATMENVYDPMSQPITPSPNNLISTNEIINPETSIDNYIYKWDTRRDILTQKATDRITKSSTYDSNVFTDGRETSTDIPLFQTTQKAPQEETPETQAETLLLQLHQLQQFNLQLQQRFNNLKQLMQNI

SEQ ID NO: 1036 - protein, 656 aa, Betatorquevirus sp.
MPWYNYWPRRRRLRRYWRRPRKTLRRRYWRRPRWVRRPYKRKLRKIVLTQFQPKTIRKSKIKGPICLFQTTNKRLVNNFDMYETSEVPEHQPGGGGWGIKVFTLEGLYSEHIYGRNIWTVTNQDLPLVRYLGCSIRFYQSEYTDYVATYSNQLPLQSSLGMYNAMQPNIHLLLKNKITMPGLKTYKRRVPYKKVFVPPPTQLENKWYFQQNIAKTPLLMTRVTACSLNKFYIDPDHINTNLTITSLNVSLFSNRQFQDATDYHPKTVGTINYYLYSTTQNPPYNGPLKLKMLIPLTDTMIYKPGINYEEYKRNNTTKGWNDWKTEYTIYSGNPFHANYLSVGEPVFLIGKKPSDLFSSEEGETSDYTTAELTKTIRYNPYNDQGQTNICYFKANFKSETTWQPPDNPDLTNENLPFWLLLWGFSDWHKKIKKHLHIDSSYILVMRHIPLALNTEYIVPLSDSFLQGKSPFSPEEQPIGADRTTWHPQFQHQAEAINTICSSGPGTTKIPDNYSVQGLMKYSFYFKWGGSPPPMSTIEDPIQQPTYPVPHNFNLTTSLQNPETDPASILWSFDQRRHTLTKKAIERLQKDTGIKTITVTGGSHFDPTPTISQEETSEESSEEEKETSLLEKLQQQQRQQRRLKLRIMKTLLKIQQLE

SEQ ID NO: 1037 - protein, 672 aa, Betatorquevirus sp.
MPWRRRRYFRRYRYRPRRWRPRKTFRTRYYRRRHWVRKKRLRKKLKKLHITEYQPSSIRKCKVKGILCLFQTSIERICYNFDMYEESIVPDRLPGGGGFSIKNLSLQSLYLEHTHGHNIFTSTNEPYPLMRYTGCTIRLYRSADVDYIVSYDNQWPLKSTMKMYNSMQPQIHYLLRHRIIVTSKKTNPNKRKLYKKVFIPPPTQMKNQWYFQSTLANIPLFMLRTSATSLDHWFIGTRDKSTNITILTLNTSVIFHRNWDKGTQPYYANVVGTQKVWLYATDQSASPIFNEIKLKNLIFLGNTQHNTQGYSYAEYLTHYHKTHSSTTVEEWQKSANWGNPFYKDYLTNPSNDHQVFASYTDILQFANKLKTSQDTTIKESTFSFTPIEMTKRLRYNPYKDNGVNNLCYFLNCKEVNGLSWDQPHNPDLTNENLPLWILLFGFSDFQKKLAKLKRIDTDYTFVIKSTYTEPTHLYIVPINISMSEGHSPYEDQANFVDDDKWFPSFQFQQEAYTDICLSGPGTARIPKGDTAEAKMHYTFHFKWGGELPDMEQIADPSEKPTYPIPNNFLSTTSLQNPTQAPETYLYKFDQRGDYITKTAAKRIRKDWSTKTDSLSITAPRFAEAAQTQDTSTETTSEEEEEEDLFQQLNQQRLKQQRLKQRIKLILQKLQTT

SEQ ID NO: 1038 - protein, 756 aa, Alphatorquevirus sp.
MPWWRWRRGWRWPRRRWRTYRRRRRLPRRRPRRAVRRPRRRRVRRRRGRGWYRGRRYSRRLYRRRYVRRKRKTLIWRQWQPQNIRKCRIRGIIPMIICGHLRSNRNYALHSDDIVEQNTPFGGALSTTSWSLKVLYDQHQRGLNRWSSSNEMLDLARYNGCRFTFYRDKVTDFIVTYDISAPYKLDKYSSPSYHPGMMMLKTKNKILIPSFNTKPKGRSTVSVKIKPPKLFLDKWYTQEDLCTVNLVTFAVSAASFTHPFCPPQTDNPCCTFQVLKDFYYPIIGYSAAETKVSSVFSNILYKHCTYYQSFLTQQFIGKIVKLPDGTLVSSLASGPSSYPEGSNINKNSIEQYNTWLSQFNTGANQHYNFCNYKPDYSKLEWLRKYYFEWETYKKASIPPDHLTPSVNWYEYHIGLFSPIFLSPFRSSSLEFPRAYQDVTYNPLVDKGVGNVIWFQYNTKADTQLTLPSCKCVIEDIPLWAAFYGYSDFVQQEIGPYTDAESVGLVCVICPYTKPPMKNPDNPMMGYVFYDSNFGAGKWIDGSGFVPLYWQTRWRPETLFQESTMRDITMTGPFSYKDELKNTVLTAKYKFNFRWGGNLLHEQTIRNPCPTNAHPSTGRQPRDVQVVDPITVGPRFVFHSWDWRRGFLSEAAIKRIRDQPLDYEAYSFMPKRPRFFPPTTGGEQLPGEQEESSSSAEENNLISFEEGQEPKAQAVHKQLLKHLRKQRVLGQRLRALYQNLQKTQAGLHINPLLFSQA

SEQ ID NO: 1039 - protein, 748 aa, Alphatorquevirus sp.
MAWRWWWRRRRPWPYRRRRWRTIRTRRPRRPVRRRRRRRRVRRRRWGRRRGRRTYYRRRFRKKKRKRKKLVLKQWNPSTIRRCTITGMIPLIVCGHTSAGRNYALRSDDFIPQSQPFGGSFSTTTWNLNVLYDEHLKFHNKWSYPNTQLDLARYLGAYFYFYRDPKTDFIVTYNTVPPFKIDKYSSPMLHPGMLMQRKKRILIPSFETRPKGKRRIRVKIKPPTLFEDKWYTQSDLCSVNLLSLAVSAASFKHPFCPPQTENICITFQVLRDFYYTQMNVTDTKGTDISDKDKAIFDNHLYKFANYYQTVHTLGQLNPTQQPALFSDRQNQGIPEKDSYLSGIGPNKLQTGNNSIYGQPNYNPDIEKLHKIRKWFFKQETTANTIHGQYGHPQNDAVDYHIGKYSPIFLSPYRTNLQFPTAYVDVTYNPLVDKGLGNKMWIQSVTKSDTQFNELNCKCVLQDIPIWAMATGYAEFIESELGLQEEVYSVYIICVICPYTQPKMYSDTNVNTGYVFYDSLFGDGKMPSGTGLVPFYYQSRWYPRLRFQLQVLHDMYICGPFSYKDDLKSTVLTAEYKFRFLWGGNMIPEQVIRNPCKAEGAGSGYTYPDRLPRDLQIADPQTMGPEWAFHTWDWRHGLFGKGALKRVSEKPDYDADYYVLPKKPRFFPPTDGSQGQEKDSDLQEISLPFPFEETLSKSQEEVQTQQQQRDLHLRLAQQQRLGQQLKHLYTQILKTQAGLHINPLLYNHV

SEQ ID NO: 1040 - protein, 742 aa, Alphatorquevirus sp.
MAWRWWWNRRPRRRWTRRRWRRLRTRRPRRPVRRRRRRRRVRRRRWGRRRGRRTFYRRRFKKRRRRRRKLTLKQWNPSTIRKCSIRGTVALVMCGHTKAGRNYALHSEDYVPSLISMGGSFSTTTWSLKALYDEFQKFHNRWSYPNTQLDLARFKGSKWTFYRDPKVDYIVTYYTVPPFKLNKLSSPFLHPGMMMQYKKKILVPSYQTKPKGKGKVSIWIKPPVLFEDKWYTQQDLCPVNLLSLAVSAASFLHPFCPPESDNMCITFQVLQDFYYTQMGVTQMTGTTLTEADNNIFKNHLYKQASYYQSLHTTGQLNATQKPANYSTNDKIPQETWLSTTGPAGFKTGNNSIYGVPNYKPDYTKLEEIRKWFFKQETTDNAIHGSYGKPVYNAVDYHIGKYSPIFLSPYRTNTQFSTAYRDITYNPNTDKGKGNHIWIQSVTKTNTTLNSQCRCHIQDIPIWAAVTGYAEFCESELNSEVHNLYICCIICPYTKPKLYNNTDPTQGYVFYDTLFGDGKMPTGTGLVPVWLQSRWYPRLIFQAQVLHDLYLTGPFSYKDDLKSFTISAKYNFKFLWGGTIIPEQVIKNPCKEQDSPFTFTGRQPRDLQVTDPQTMGPEWVFHTWDWRRGLFGENAIKRVSEQPSHDADYYAPPKKPRLFPPTDLQEQESAFDFTPQELKILSEAPQQSEEEENQEEEQQLQQRELQLRLQEQFRIRHQLQHLFVQVLKTQAGLHINPLFLNPV

SEQ ID NO: 1041 - protein, 735 aa, Alphatorquevirus sp.
MAYWWWRRRRRRPWRRRRRWRLRRPRRRRATYRRRRGRRRVRKRRGRWRRTYRRWRRRKGRRRHKKKIIMKQWQPNFIRHCFIVGFMPLIFCGENTTSQNFATHSDDLVAKGPYGGGMTTTKFTLRILYDEHLRFMNFWTVSNEDLDLCRYLGVKLTFYRHPTVDFMVQIHTQPPFLDTELTGPSIHPGMMILNKKKIFVPSLKTRPNRKHKVRVKIGPPRLYEDKWYPQSELCDTTLLTVYATACDLQFPFGSPLTDNPCVNFQVLGSAYKKHLSILSTNDSKNLTHYETALFSKTAYYNTFETIAQLKPTGQTTGVTPTWQDVQNTTNLDSSGTNQATSNDTWYYGNTYKDNIKDLTKKARDRYLAATQKAFPAYATLQSPLYEYHGGCYSSIFLSAGRAYFETLGAYSDIIYNPFVDKGIGNMVWIDYLSKEDAVYTPRTSKCLIRDQPLWASFNGYSEFCSKSTGDGAVNINTRVVIKCPYTTPMLADNSNENQGFVPYSLNFGKGKMKGGSSQVPLSWRCKWYVMMYHQQEFMEAIVQSGPWAYKGDEKSAVLAMKYVFNWKWGGNPISGQVVRNPCKTSSSSREPRSIQAVDPKRVAPPLVFHSWDYRRGLFGHASIKRMSEESATPTLFTGPAGKRPRRDTSVQQAEEDQQKESSSFRVQRQLQPWIHSSQETQSSQEEMQAQGTVQEQLIQQLGEQRALRKQLEFLASQVLKVQAGQHIHPLLSSQA

SEQ ID NO: 1042 - protein, 662 aa, Gammatorquevirus sp.
MPFWWRRRRKFWTNNRFNYTKRRRYRKRWPRRRRRRRPYRRPIRRRRRKLRKVKRKKKSLIVRQWQPDSIRTCKIIGQSAIVVGAEGKQMYCYTVNKLINVPPKTPYGGGFGVDQYTLKYLYEEYRFAQNIWTQSNVLKDLCRYINVKLIFYRDNKTDFVLSYDRNPPFQLTKFTYPGAHPQQIMLQKHHKFILSQMTKPNGRLTKKLKIKPPKQMLSKWFFSKQFCKYPLLSLKASALDLRHSYLGCCNENPQVFFYYLNHGYYIQTNWGAQSTTAYRPNSSVTETTYYKYKNEKKNIHIKSSEYSESISYDKGYFQSSFLQTQCIYTSAAGDTCAAEKPLGIAIYNPVKDNGDGNMIYLVSTLANTWDQPPKDSAILIQGVPIWLGLFGYLDYCRQIKADKTWLDSHILVMQSPAIFTYPNPGAGKWYCPLSQSFINGNGPFNQPPTLLQKAKWFPQIQYQQEIINSFVESGPFVPKYANQTESNWELKYKYIFTFKWGGPQFHEPEIADPSKQEQYDVPDTFYQTIQIEDPEGQDPRSLIHDWDYRRGFVKERSLKRMSTYFSTHTDQQATSEEDIPKKKKRIGPQLTVPQQKEEETLSCLLSLCKKDTFQETETQEDLQQLIKQQQEQQLLLKRNILQLIHKLKENQQMLQLHTGMLP

SEQ ID NO: 1043 - protein, 660 aa, Gammatorquevirus sp.
MPFWWGRRNRFWYKNNYRRRAYRRKRKRWPKRRRRNFRRPTRRRRRRRRKQKVRKKKQTLTLKVWQPDTIRKCKIKGTGIHIVGGNGTQYRCFTANRFDWTPPLMPGGGGFGVEKFTLQHLYNEHLLSNNIWTQSNKMLDLVRYTGCRIKFYRHETIDFIASYNRNLPFNLEKYTYADSHPNSLLHSRHKKLIPSLKTRRSGKRYITMKIKPPKPVSNKWFFQETFSSTPLFKLTTSIIDLRYPHISQIGSNELVSLFCLNTDFYANAGWGNVHPPGTHSKWYVPYHNIAAITTVVIKGRDTQITVDGSDYKKSVSYETGYFQPKLLQAEKIKNTAMENIPIQASRYNPTKDDGVGNKLWLVNVLNYSFAPPKTDADLYIENLPLWQLVWGFTDFVKQTKKDSTFLNTYYAAFESKYLEPAVSTSKIYIPIDMSFIQGKGPYDSYVSNYRKQNWYPTLNAQLKTLNAIAQAGPFVPKLDNIFLSSWELYSNYTFYFKFGGSELPEPDTVNPQDQGTYVIPTNIQKAIQVSNPQKQSPYTTLHSWDYRRGLITSSAFKRMCENQTTDTEFEPDTETIPKKKKKIQHGNQLQRAEEETQEIQNCLLSLCEENTFQETPQTSLLELIQQQQQQQQQLKHNLLKLLSDLKLKQRSLQLQTGLIH

SEQ ID NO: 1044 - protein, 663 aa, Gammatorquevirus sp.
MPFWWRRRRKPWYGKWRFRRRRRYNAPRRKRKFYQRRRYRRTNRRRRKYKVRRKKRKTIAIRQWQPESITKCKIIGFSNLVLGAEGTQYLCWTSQQEDYTQPKAPGGGGFGCELINLDWLYSEWVKHNNIWTKTNTNKDLVRYTGAYIYFLRHPTIDFVIAYSLQPPFELTKFTYPDIQPQNLLLRKRKRIILSLASKPHGKRFVKVKIRPPKQMITKWFFQHQFSGYGLVQLSAAAATFRHPNIGPKSQNQMVTIYFLDTTIFNRPNWAQAQEDAWTPAGTHRTWTFEYQVKGQTQPKTVQMPPSTPQDPYKTKYATSISRDYGWWQKAILNSTKVTIDGTMYSNRTVYSARYNPNEDTGQGNAVYLVSLLQSKWTAPSDDRLVIVGQPLWMCMYGLYSYYQDLLKDKNFNTHYMFVVRSPAIKPITVSRQDFYPFIDWDFINGVLPWEEYLSEKIKATWYPQATFQTTTINALVESGPFVPRLTNIPDSTWELVYKYKFFFKWGGPQTHDDHVQDPKHQPDYDTPDTLHQTIQIGDPKKQTPESIIHEWDYRRGFITQTALKRMSENIQTDTDFQSDDSASPRKKRKVTKKMPITSQKEENIKKCLLSLCQEETQETPENLQDLIQQQQQQQHLLKRNLLQLLTHLKQQQRYLSLQTGILD

SEQ ID NO: 1045 - protein, 662 aa, Gammatorquevirus sp.
MPFWWPRRRRWWATTRFKRRKPRYPKRRRRRRYKRKYRRAPRRRRRRRRKVRRKKPNLIVRQWQPDSIVLCKIKLFGSLVLGAEGTQYLCYTNEKGAWTPPQQPGGGGFGIETYSLSYLYDQYLLRRCIWTKSNDYKDLCRYLRCKITFFRHPKIDFVVNYARQPPFLLEKPTYMNCHPIALLLSQHKRVILSKRSDPKAKNRVTLIIKPPKQMITKWFFTKQFALFDLLQIQAAACTLNYPKLGCCNENLIISIFYLNPQFFQDSQWAQYTTSPYKPYQTIDTQLTYHYKIGNQTGQYKPEFLNKTGQQAYYESISLDKGWFSPKIMNTQYITQSGSETHLAAIPVGAARYNPEIDDGKGNEIYLVSVTTGHYTKPSEDNLVFRGLPLWLMFFGLYSFIKKIKTASFLPLHMFVVKSEYILPKPKDPQKQYFPFVDLNFINGKNPYNSYISSTQRQTWYPTCEHQVETINAFVKCGPFVPKLDNDRDSTWELPFKSTFYFKWGGPQMPNQNVSDPKDKTSYIVPDTVQEGLQITNPLKQKAESLLHEWDYRRGLIKTKAFKRMLENLSIESDVYPSAETPRKRQRVENSLHYQEEENQEIKTCLLSLCESSSSEKEEEEKNLLHLIQQQQQQQKQLKLQLLTLIKDLKEKQNALQLQTGVI

SEQ ID NO: 1046 - protein, 672 aa, Gammatorquevirus sp.
MPFWWRRRRKFWRGRRRPFYKRTTKRRRRRVRYRQRFRRPYRRRRRRRKHKVRRKRQKLPIYQWQPDSIRKCKIKGVGTLILGAHGKQFVCYTDVQKKAPPPKAPGGGGFACEQFSLGGLYEQYKFRNNIWTASNINMDLCRYLGVRMIFYRHPDIDFILNYDRQPPFVLDQLTYPLCHPQNLLLGKHKILLLSKATKPNGKLRKKVFIKPPKQMLNKWFFQEQFQSAPLLSIRASICSLQYPHIGCCMPNRTVTFFALNPGFYQQGNWAYDGGETNPYKPWPHIPKKLYFWDVATIPASWMEQEKAKHRVEIDLSTYSQSIDYQKGYFTSKVLQAKLVTQDSTGTSTIANTPLNIARYNPAIDTGKGNTIWLHSNLTTAYTKPAVDKTVILEGLPLWMMLFGWLSWVQHVKGAPDFYLSYTLCVESPAIEIATKGETINPVIPLDASFINGNAPYNQTISQKDLTHWYPDIYNQTEIINSIVTSGPYIPKLDNIKNSTWELHYYYQFFFKWGGPEITDQPVADPSKQPVYDALDKQQQGIQVRNPAKQKYATLMHPWDIRRGIITNKALKRMQQNLSIDSTFEAGSTPAKKPRITGPCFTALQEIHQEERSCLQTLCKESTSQEAQEETDIQLLIAQQQQKQRELKYNLLKLISYMKEQQNMLKLHTGMLN

SEQ ID NO: 1047 - protein, 39 aa, Torque teno midi virus
CSEEEEESNLFERLLRQRTKQLQLKRRIIQTLKDLQKLE

SEQ ID NO: 1048 - protein, 150 aa, Betatorquevirus sp.
MPWWYRRRSYNPWRRRNWFRRPRKTIYRRYRRRRRWPTYVVPNNFNETTSLQNPTTRPEHFLYSFDERRGQLTEKATKRLLKDWETKETSLLSTEYRFAEPTQTQAPQEDPSSEEEEESNLFERLLRQRTKQLQLKRRIIQTLKDLQKLE

SEQ ID NO: 1049 - protein, 45 aa, Betatorquevirus sp.
MPWWYRRRSYNPWRRRNWFRRPRKTIYRRYRRRRRWVRRKPFYKR

SEQ ID NO: 1050 - protein, 184 aa, Betatorquevirus sp.
KIKRLNIVEWQPKSIRKCRIKGMLCLFQTTEDRLSYNFDMYEESIIPEKLPGGGGFSIKNISLYALYQEHIHAHNIFTHTNTDRPLARYTGCSLKFYQSKDIDYVVTYSTSLPLRSSMGMYNSMQPSIHLMQQNKLIVPSKQTQKRRKPYIKKHISPPTQMKSQWYFQHNIANIPLLMIRTTAL

SEQ ID NO: 1051 - protein, 156 aa, Betatorquevirus sp.
TLDNYYIGSRQLSTNVTIHTLNTTYIQNRDWGDRNKTYYCQTLGTQRYFLYGTHSTAQNINDIKLQELIPLTNTQDYVQGFDWTEKDKHNITTYKEFLTKGAGNPFHAEWITAQNPVIHTANSPTQIEQIYTASTTTFQNKKLTDLPTPGYIFITP

SEQ ID NO: 1052 - protein, 158 aa, Betatorquevirus sp.
TVSLRYNPYKDLAERNKCYFVRSKINAHGWDPEQHQELINSDLPQWLLLFGYPDYIKRTQNFALVDTNYILVDHCPYTNPEKTPFIPLSTSFIEGRSPYSPSDTHEPDEEDQNRWYPCYQYQQESINSICLSGPGTPKIPKGITAEAKVKYSFNFKWG

SEQ ID NO: 1053 - protein, 129 aa, Betatorquevirus sp.
GDLPPMSTITNPTDQPTYVVPNNFNETTSLQNPTTRPEHFLYSFDERRGQLTEKATKRLLKDWETKETSLLSTEYRFAEPTQTQAPQEDPSSEEEEESNLFERLLRQRTKQLQLKRRIIQTLKDLQKLE

SEQ ID NO: 1054 - protein, 4 aa, synthetic construct
NANP

SEQ ID NO: 1055 - protein, 409 aa, synthetic construct
ANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLNENTTSGFLGPLLVLQAGFFLLTRILTIPQSLDSWWTSLNFQGGAPTCPGQNSQSPTSNHSPTSCPPICPGYRWMCLRRFIIFLFILLLCLIFLLVLLDYQGMLPVCPLLPGTSTTGTGPCRTCTIPAQGTSMFPSCCCTKPSDGNCTCIPIPSSWAFARFLWEWASVRFSWLSLLVPFVQWFAGLSPTVWLSVIWMMWYRGPSLYNTLSPFLPLLPISFCLWVYI

SEQ ID NO: 1056 - protein, 410 aa, synthetic construct
ANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLNENTTSGFLGPLLVLQAGFFLLTRILTIPQSLDSWWTSLNFQGGAPTCPGQNSQSPTSNHSPTSCPPICPGYRWMCLRRFIIFLFILLLCLIFLLVLLDYQGMLPVCPLLPGTSTTGTGPCRTCTIPAQGTSMFPSCCCTKPSDGNCTCIPIPSSWAFARFLWEWASVRFSWLSLLVPFVQWFAGLSPTVWLSVIWMMWYRGPSLYNTLSPFLPLLPISFCLWVYIC

SEQ ID NO: 1057 - protein, 218 aa, synthetic construct
MKIKTGARILALSALTTMMFSASALAANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLNPHHHHHHA

SEQ ID NO: 1058 - protein, 219 aa, synthetic construct
MKIKTGARILALSALTTMMFSASALAANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLNPHHHHHHAC

SEQ ID NO: 1059 - protein, 24 aa, synthetic construct
NPNANPNANPNANPNANPNANPNA

SEQ ID NO: 1060 - protein, 40 aa, synthetic construct
ANPNKNNQGNGQGHNMPNDPNRNVDENANANSAVKNNNNE

SEQ ID NO: 1061 - protein, 66 aa, synthetic construct
EPSDKHIKEYLNKIQNSLSTEWSPSSVTSGNGIQVRIKPGSANKPKDELDYANDIEKKISKMEKSS

SEQ ID NO: 1062 - protein, 266 aa, synthetic construct
MRGTPKTHLLAFSLLCLLSKVRTQLCPTPCTCPWPPPRCPLGVPLVLDGCGCCRVCARRLGEPCDQLHVCDASQGLVCQPGAGPGGRGALCLLAEDDSSCEVNGRLYREGETFQPHCSIRCRCEDGGFTCVPLCSEDVRLPSWDCPHPRRVEVLGKCCPEWVCGQGGGLGTQPLPAQGPQFSGLVSSLPPGVPCPEWSTAWGPCSTTCGLGMATRVSNQNRFCRLETQRRLCLSRPCPPSRGRSPQNSAFGGGGSGGGGSGGGGSC

SEQ ID NO: 1063 - protein, 477 aa, synthetic construct
MKIKTGARILALSALTTMMFSASALASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGGGSGGGGSGGGGSGGGGSC

SEQ ID NO: 1064 - protein, 247 aa, synthetic construct
MKIKTGARILALSALTTMMFSASALASDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKGGGGSGGGGSGGGGSC

SEQ ID NO: 1065 - protein, 273 aa, synthetic construct
MKIKTGARILALSALTTMMFSASALAEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGGGGSGGGGSGGGGSC

SEQ ID NO: 1066 - protein, 499 aa, synthetic construct
MKIKTGARILALSALTTMMFSASALAEVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKKDELGGGGSGGGGSGGGGSC

SEQ ID NO: 1067 - protein, 537 aa, synthetic construct
MKIKTGARILALSALTTMMFSASALAEVQLVESGGGLVQPGGSLRLSCAASGYDFTHYGMNWVRQAPGKGLEWVGWINTYTGEPTYAADFKRRFTFSLDTSKSTAYLQMNSLRAEDTAVYYCAKYPYYYGTSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHLGGSSGSGSGSTGTSSSGTGTSAGTTGTSASTSGSGSGGGGGSGGGGSAGGDIQLTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSC

SEQ ID NO: 1068 - protein, 24 aa, synthetic construct
NANPNANPNANPNANPNANPNANP

SEQ ID NO: 1069 - protein, 6 aa, synthetic construct
HHHHHH

SEQ ID NO: 1070 - DNA, 19 bp, synthetic construct
  source 1..19: mol_type = other DNA
tgaagctggggtctttaac

SEQ ID NO: 1071 - DNA, 21 bp, synthetic construct
  source 1..21: mol_type = other DNA
tctacctaggtgcaaagggcc

SEQ ID NO: 1072 - protein, 13 aa, synthetic construct
KIKRLNIVEWQPK

SEQ ID NO: 1073 - protein, 18 aa, synthetic construct
SPSDTHEPDEEDQNRWYP

SEQ ID NO: 1074 - protein, 38 aa, synthetic construct
SEEEEESNLFERLLRQRTKQLQLKRRIIQTLKDLQKLE

SEQ ID NO: 1075 - protein, 4 aa, synthetic construct
  SITE 4: note = Proline N-hydroxysuccinimide
NANP

(19) United States
(12) Patent Application Publication
(10) Pub. No.: US 2025/0171807 A1
(43) Pub. Date: May 29, 2025

(54) SURFACE-MODIFIED VIRAL PARTICLES AND MODULAR VIRAL PARTICLES

(71) Applicant: FLAGSHIP PIONEERING INNOVATIONS V, INC., Cambridge, MA (US)

(72) Inventors: Simon Delagrave, Sudbury, MA (US); Kurt Adam Swanson, West Newton, MA (US); Noah Robert Cohen, Ashland, MA (US); Shu-Hao Liou, Cambridge, MA (US); Amir Khan, Brookline, MA (US)

(21) Appl. No.: 19/046,064

(22) Filed: Feb. 5, 2025

Related U.S. Application Data
(63) Continuation of application No. 18/719,065, filed on Jun. 12, 2024.
(60) Provisional application No. 63/344,029, filed on May 19, 2022.
(60) Provisional application No. 63/289,975, filed on Dec. 15, 2021.
(60) Provisional application No. 63/289,967, filed on Dec. 15, 2021.

(51) Int. Cl. C12N 15/86; C07K 14/005; C12N 7/00

(52) U.S. Cl. CPC C12N 15/86; C07K 14/005; C12N 7/00; C12N 2750/00022; C12N 2750/00023; C12N 2750/00043; C12N 2750/00051

(57) ABSTRACT

This invention relates generally to anellovectors, anelloVLPs, and compositions and uses thereof.

### CROSS REFERENCE TO RELATED APPLICATIONS

This application is a Continuation of U.S. patent application Ser. No. 18/719,065, filed Jun. 12, 2024, which is a U.S. National Phase Application under 35 U.S.C. § 371 of International Application No. PCT/US2022/081572 filed Dec. 14, 2022, which claims the benefit of U.S. Provisional Application No. 63/289,967, filed Dec. 15, 2021, U.S. Provisional Application No. 63/289,975, filed Dec. 15, 2021, and U.S. Provisional Application No. 63/344,029, filed May 19, 2022. The contents of the aforesaid applications are hereby incorporated by reference in their entirety.

### SEQUENCE LISTING

The instant application contains a Sequence Listing which has been submitted electronically in XML format and is hereby incorporated by reference in its entirety. Said XML copy, created on Feb. 4, 2025, is named V2057-702020FT_SL.xml and is 1,080,562 bytes in size.

### BACKGROUND

There is an ongoing need to develop suitable vectors to deliver therapeutic agents to patients.

### SUMMARY

The present disclosure provides an anellovector, e.g., a synthetic anellovector, which can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). The anellovector generally comprises on its exterior surface (e.g., attached to a proteinaceous exterior) a surface moiety as described herein. In some embodiments, an anellovector (e.g., particle, e.g., a viral particle, e.g., an Anellovirus particle) comprises a genetic element (e.g., a genetic element comprising a therapeutic DNA sequence) encapsulated in a proteinaceous exterior (e.g., a proteinaceous exterior comprising an Anellovirus capsid protein, e.g., an Anellovirus ORF1 molecule or a polypeptide encoded by an Anellovirus ORF1 nucleic acid, e.g., as described herein), which is capable of introducing the genetic element into a cell (e.g., a mammalian cell, e.g., a human cell). In some embodiments, the anellovector is a particle comprising a proteinaceous exterior comprising a polypeptide encoded by an Anellovirus ORF1 nucleic acid (e.g., an ORF1 nucleic acid of Betatorquevirus, e.g., as described herein). The genetic element of an anellovector of the present disclosure is typically a circular and/or single-stranded DNA molecule (e.g., circular and single stranded), and generally includes a protein binding sequence that binds to the proteinaceous exterior enclosing it, or a polypeptide attached thereto, which may facilitate enclosure of the genetic element within the proteinaceous exterior and/or enrichment of the genetic element, relative to other nucleic acids, within the proteinaceous exterior. In some instances, the genetic element is circular or linear. In some instances, the genetic element comprises or encodes an effector (e.g., a nucleic acid effector, such as a non-coding RNA, or a polypeptide effector, e.g., a protein), e.g., which can be expressed in the cell. In some embodiments, the effector is a therapeutic agent or a therapeutic effector, e.g., as described herein. In some instances, the effector is an endogenous effector or an exogenous effector, e.g., to a wild-type Anellovirus or a target cell. In some embodiments, the effector is exogenous to a wild-type Anellovirus or a target cell. In some embodiments, the anellovector can deliver an effector into a cell by contacting the cell and introducing a genetic element encoding the effector into the cell, such that the effector is made or expressed by the cell. In certain instances, the effector is an endogenous effector (e.g., endogenous to the target cell but, e.g., provided in increased amounts by the anellovector). In other instances, the effector is an exogenous effector. The effector can, in some instances, modulate a function of the cell or modulate an activity or level of a target molecule in the cell. For example, the effector can decrease levels of a target protein in the cell. In another example, the anellovector can deliver and express an effector, e.g., an exogenous protein, in vivo. Anellovectors can be used, for example, to deliver genetic material to a target cell, tissue or subject; to deliver an effector to a target cell, tissue or subject; or for treatment of diseases and disorders, e.g., by delivering an effector that can operate as a therapeutic agent to a desired cell, tissue, or subject. In some instances, the anellovector is made by in vitro assembly. In vitro assembly of an anellovector generally involves the formation of a proteinaceous exterior enclosing a genetic element, which occurs outside of a host cell (e.g., in a cell-free suspension, lysate, or supernatant). In vitro assembly may, in some instances, utilize components generated in a host cell but does not generally require a host cell for particle assembly.

The present disclosure provides an anelloVLP, e.g., a synthetic anelloVLP, which can be used as a delivery vehicle, e.g., for delivering genetic material, for delivering an effector, e.g., a payload, or for delivering a therapeutic agent or a therapeutic effector to a eukaryotic cell (e.g., a human cell or a human tissue). The anelloVLP generally comprises on its exterior surface (e.g., attached to a proteinaceous exterior) a surface moiety as described herein. In some embodiments, the surface moiety comprises the effector. In some embodiments, the surface moiety comprises a targeting agent (e.g., an agent that targets the anelloVLP to a target cell or tissue). In some embodiments, an anelloVLP (e.g., particle, e.g., a viral particle, e.g., an Anellovirus particle) comprises a proteinaceous exterior (e.g., a proteinaceous exterior comprising an Anellovirus capsid protein, e.g., an Anellovirus ORF1 molecule or a polypeptide encoded by an Anellovirus ORF1 nucleic acid, e.g., as described herein). In some embodiments, the anelloVLP is a particle comprising a proteinaceous exterior comprising a polypeptide encoded by an Anellovirus ORF1 nucleic acid (e.g., an ORF1 nucleic acid of Betatorquevirus, e.g., as described herein). In some embodiments, the proteinaceous exterior encloses an effector. In some embodiments, the effector is a therapeutic agent or a therapeutic effector, e.g., as described herein. In some instances, the effector is an endogenous effector or an exogenous effector, e.g., to a wild-type Anellovirus or a target cell. In some embodiments, the effector is exogenous to a wild-type Anellovirus or a target cell. In some embodiments, the anelloVLP can deliver an effector into a cell by contacting the cell and introducing the effector into the cell. In certain instances, the effector is an endogenous effector (e.g., endogenous to the target cell but, e.g., provided in increased amounts by the anelloVLP). In other instances, the effector is an exogenous effector. The effector can, in some instances, modulate a function of the cell or modulate an activity or level of a target molecule in the cell. For example, the effector can decrease levels of a target protein in the cell. In another example, the anelloVLP can deliver an effector, e.g., an exogenous protein, in vivo. AnelloVLPs can be used, for example, to deliver an effector to a target cell, tissue or subject; or for treatment of diseases and disorders, e.g., by delivering an effector that can operate as a therapeutic agent to a desired cell, tissue, or subject. In some instances, the anelloVLP is made by in vitro assembly.

In vitro assembly of an anelloVLP generally involves the formation of a proteinaceous exterior in connection with an effector (e.g., the proteinaceous exterior enclosing the effector), which occurs outside of a host cell (e.g., in a cell-free suspension, lysate, or supernatant). In vitro assembly of an anelloVLP may, in some instances, utilize components generated in a host cell but does not generally require a host cell for particle assembly.

The invention further provides synthetic anellovectors and synthetic anelloVLPs. A synthetic anellovector or synthetic anelloVLP has at least one structural difference compared to a wild-type virus (e.g., a wild-type Anellovirus, e.g., a described herein), e.g., a deletion, insertion, substitution, modification (e.g., enzymatic modification), relative to the wild-type virus. Generally, synthetic anellovectors and synthetic anelloVLPs include a proteinaceous exterior, which can be used for delivering an effector (e.g., an exogenous effector or an endogenous effector) into eukaryotic (e.g., human) cells. In some embodiments, the anellovector or anelloVLP does not cause a detectable and/or an unwanted immune or inflammatory response, e.g., does not cause more than a 1%, 5%, 10%, 15% increase in a molecular marker(s) of inflammation, e.g., TNF-alpha, IL-6, IL-12, IFN, as well as B-cell response e.g. reactive or neutralizing antibodies, e.g., the anellovector or anelloVLP may be substantially non-immunogenic to the target cell, tissue or subject.

In an aspect, the invention features an anellovector comprising: (i) a genetic element comprising a promoter element and a sequence encoding an effector (e.g., an endogenous or exogenous effector), and a protein binding sequence (e.g., an exterior protein binding sequence, e.g., a packaging signal); and (ii) a proteinaceous exterior; wherein the genetic element is enclosed within the proteinaceous exterior (e.g., a capsid); and wherein the anellovector is capable of delivering the genetic element into a eukaryotic (e.g., mammalian, e.g., human) cell. In some embodiments, the anellovector comprises a surface moiety (e.g., a surface moiety having effector and/or targeting function), e.g., displayed on the exterior surface of the anellovector (e.g., as described herein). In some embodiments, the surface moiety comprises the effector.

In some embodiments, the genetic element is a single-stranded and/or circular DNA. Alternatively or in combination, the genetic element has one, two, three, or all of the following properties: is circular, is single-stranded, it integrates into the genome of a cell at a frequency of less than about 0.0001%, 0.001%, 0.005%, 0.01%, 0.05%, 0.1%, 0.5%, 1%, 1.5%, or 2% of the genetic element that enters the cell, and/or it integrates into the genome of a target cell at less than 1, 2, 3, 4, 5, 6, 7, 8, 9, 10, 15, 20, 25, or 30 copies per genome. In some embodiments, integration frequency is determined as described in Wang et al. (2004, Gene Therapy 11: 711-721, incorporated herein by reference in its entirety). In some embodiments, the genetic element is enclosed within the proteinaceous exterior. In some embodiments, the anellovector is capable of delivering the genetic element into a eukaryotic cell. In some embodiments, the genetic element comprises a nucleic acid sequence (e.g., a nucleic acid sequence of between 300-4000 nucleotides, e.g., between 300-3500 nucleotides, between 300-3000 nucleotides, between 300-2500 nucleotides, between 300-2000 nucleotides, between 300-1500 nucleotides) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Torque Teno virus (TTV), Torque Teno mini virus (TTMV), or TTMDV sequence, e.g., a wild-type Anellovirus sequence as listed in any one of Tables A1-A25 or N1-N25). In some embodiments, the genetic element comprises a nucleic acid sequence (e.g., a nucleic acid sequence of at least 300 nucleotides, 500 nucleotides, 1000 nucleotides, 1500 nucleotides, 2000 nucleotides, 2500 nucleotides, 3000 nucleotides or more) having at least 75% (e.g., at least 75, 76, 77, 78, 79, 80, 90, 91, 92, 93, 94, 95, 96, 97, 98, 99, or 100%) sequence identity to a sequence of a wild-type Anellovirus (e.g., a wild-type Anellovirus sequence as described herein, e.g., as listed in any one of Tables A1-A25 or N1-N25). In some embodiments, the nucleic acid sequence is codon-optimized, e.g., for expression in a mammalian (e.g., human) cell. In some embodiments, at least 50%, 60%, 70%, 80%, 90%, 95%, 96%, 97%, 98%, 99%, or 100% of the codons in the nucleic acid sequence are codon-optimized, e.g., for expression in a mammalian (e.g., human) cell.

In an aspect, the invention features an anelloVLP comprising a proteinaceous exterior (e.g., a capsid) and an effector; wherein the anelloVLP is capable of delivering the effector into a eukaryotic (e.g., mammalian, e.g., human) cell. In some embodiments, the effector is comprised in a surface moiety, e.g., displayed on the exterior surface of the anelloVLP (e.g., as described herein).

In an aspect, the invention features an infectious (to a human cell) particle comprising an Anellovirus capsid (e.g., a capsid comprising an Anellovirus ORF, e.g., ORF1, polypeptide). In some embodiments, the infectious particle encapsulates a genetic element comprising a protein binding sequence that binds to the capsid and a heterologous (to the Anellovirus) sequence encoding a therapeutic effector. In some embodiments, the particle is capable of delivering the genetic element into a mammalian, e.g., human, cell. In some embodiments, the genetic element has less than about 6% (e.g., less than 6%, 5.5%, 5%, 4.5%, 4%, 3.5%, 3%, 2.5%, 2%, 1.5%, or less) identity to a wild type Anellovirus. In some embodiments, the genetic element has no more than 1.5%, 2%, 2.5%, 3%, 3.5%, 4%, 4.5%, 5%, 5.5% or 6% identity to a wild type Anellovirus. In some embodiments, the genetic element has at least about 2% to at least about 5.5% (e.g., 2 to 5%, 3% to 5%, 4% to 5%) identity to a wild type Anellovirus. In some embodiments, the genetic element has greater than about 2000, 3000, 4000, 4500, or 5000 nucleotides of non-viral sequence (e.g., non Anellovirus genome sequence). In some embodiments, the genetic element has greater than about 2000 to 5000, 2500 to 4500, 3000 to 4500, 2500 to 4500, 3500, or 4000, 4500 (e.g., between about 3000 to 4500) nucleotides of non-viral sequence (e.g., non Anellovirus genome sequence). In some embodiments, the genetic element is a single-stranded, circular DNA. Alternatively or in combination, the genetic element has one, two or 3 of the following properties: is circular, is single stranded, it integrates into the genome of a cell at a frequency of less than about 0.001%, 0.005%, 0.01%, 0.05%, 0.1%, 0.5%, 1%, 1.5%, or 2% of the genetic element that enters the cell, it integrates into the genome of a target cell at less than 1, 2, 3, 4, 5, 6, 7, 8, 9, 10, 15, 20, 25, or 30 copies per genome or integrates at a frequency of less than about 0.0001%, 0.001%, 0.005%, 0.01%, 0.05%, 0.1%, 0.5%, 1%, 1.5%, or 2% of the genetic element that enters the cell. In some embodiments, integration frequency is determined as described in Wang et al. (2004, Gene Therapy 11: 711-721, incorporated herein by reference in its entirety).

Also described herein are viral vectors and viral particles based on Anelloviruses, which can be used to deliver an agent (e.g., an exogenous effector or an endogenous effector, e.g., a therapeutic effector) to a cell (e.g., a cell in a subject to be treated therapeutically). In some embodiments, Anelloviruses can be used as effective delivery vehicles for introducing an agent, such as an effector described herein, to a target cell, e.g., a target cell in a subject to be treated therapeutically or prophylactically.

In an aspect, the invention features a polypeptide (e.g., a synthetic polypeptide, e.g., an ORF1 molecule) comprising (e.g., in series):
(i) a first region comprising an arginine-rich region, e.g., amino acid sequence having at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof),
(ii) a second region comprising a jelly-roll domain, e.g., an amino acid sequence having at least 30% (e.g., at least about 30, 35, 40, 50, 60, 70, 80, 90, 95, 96, 97, 98, 99, or 100%) sequence identity to a jelly-roll region sequence described herein or a sequence comprising at least 6 beta strands,
(iii) a third region comprising an amino acid sequence having at least 30% (e.g., at least about 30, 35, 40, 50, 60, 70, 80, 90, 95, 96, 97, 98, 99, or 100%) sequence identity to an N22 domain sequence described herein,
(iv) a fourth region comprising an amino acid sequence having at least 70% (e.g., at least about 70, 80, 90, 95, 96, 97, 98, 99, or 100%) sequence identity to an Anellovirus ORF1 C-terminal domain (CTD) sequence described herein, and
(v) optionally wherein the polypeptide has an amino acid sequence having less than 100%, 99%, 98%, 95%, 90%, 85%, 80% sequence identity to a wild type Anellovirus ORF1 protein described herein.

In some embodiments, the polypeptide comprises at least about 70, 80, 90, 95, 96, 97, 98, 99, or 100% sequence identity to an Anellovirus ORF1 molecule as described herein (e.g., as listed in any one of Tables A1-A25). In some embodiments, the polypeptide comprises at least about 70, 80, 90, 95, 96, 97, 98, 99, or 100% sequence identity to a subsequence (e.g., an arginine (Arg)-rich domain, a jelly-roll domain, a hypervariable region (HVR), an N22 domain, or a C-terminal domain (CTD)) of an Anellovirus ORF1 molecule as described herein (e.g., as listed in any one of Tables A1-A25). In one embodiment, the amino acid sequences of the (i), (ii), (iii), and (iv) region have at least 90% sequence identity to their respective references and wherein the polypeptide has an amino acid sequence having less than 100%, 99%, 98%, 95%, 90%, 85%, 80% sequence identity to a wild type Anellovirus ORF1 protein described herein.

In an aspect, the invention features a complex comprising a polypeptide as described herein (e.g., an Anellovirus ORF1 molecule as described herein) and a genetic element comprising a promoter element and a nucleic acid sequence (e.g., a DNA sequence) encoding an effector (e.g., an exogenous effector or an endogenous effector), and a protein binding sequence.

The present disclosure further provides nucleic acid molecules (e.g., a nucleic acid molecule that includes a genetic element as described herein, or a nucleic acid molecule that includes a sequence encoding a proteinaceous exterior protein as described herein). A nucleic acid molecule of the invention may include one or both of (a) a genetic element as described herein, and (b) a nucleic acid sequence encoding a proteinaceous exterior protein as described herein.

In an aspect, the invention features an isolated nucleic acid molecule comprising a genetic element comprising a promoter element operably linked to a sequence encoding an effector, e.g., a payload, and an exterior protein binding sequence. In some embodiments, the exterior protein binding sequence includes a sequence at least 75% (at least 80%, 85%, 90%, 95%, 97%, 100%) identical to a 5′UTR sequence of an Anellovirus, as disclosed herein. In some embodiments, the genetic element is a single-stranded DNA, is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome or integrates at a frequency of less than about 0.001%, 0.005%, 0.01%, 0.05%, 0.1%, 0.5%, 1%, 1.5%, or 2% of the genetic element that enters the cell. In some embodiments, integration frequency is determined as described in Wang et al. (2004, Gene Therapy 11: 711-721, incorporated herein by reference in its entirety). In embodiments, the effector does not originate from TTV and is not an SV40-miR-S1. In embodiments, the nucleic acid molecule does not comprise the polynucleotide sequence of TTMV-LY2.

In embodiments, the promoter element is capable of directing expression of the effector in a eukaryotic (e.g., mammalian, e.g., human) cell.

In some embodiments, the nucleic acid molecule is circular. In some embodiments, the nucleic acid molecule is linear. In some embodiments, a nucleic acid molecule described herein comprises one or more modified nucleotides (e.g., a base modification, sugar modification, or backbone modification).

In some embodiments, the nucleic acid molecule comprises a sequence encoding an ORF1 molecule (e.g., an Anellovirus ORF1 protein, e.g., as described herein). In some embodiments, the nucleic acid molecule comprises a sequence encoding an ORF2 molecule (e.g., an Anellovirus ORF2 protein, e.g., as described herein). In some embodiments, the nucleic acid molecule comprises a sequence encoding an ORF3 molecule (e.g., an Anellovirus ORF3 protein, e.g., as described herein). In an aspect, the invention features a genetic element comprising one, two, or three of: (i) a promoter element and a sequence encoding an effector, e.g., an exogenous or endogenous effector; (ii) at least 72 contiguous nucleotides (e.g., at least 72, 73, 74, 75, 76, 77, 78, 79, 80, 90, 100, or 150 nucleotides) having at least 75% (e.g., at least 75, 76, 77, 78, 79, 80, 90, 91, 92, 93, 94, 95, 96, 97, 98, 99, or 100%) sequence identity to a wild-type Anellovirus sequence; or at least 100 (e.g., at least 300, 500, 1000, 1500) contiguous nucleotides having at least 72% (e.g., at least 72, 73, 74, 75, 76, 77, 78, 79, 80, 90, 91, 92, 93, 94, 95, 96, 97, 98, 99, or 100%) sequence identity to a wild-type Anellovirus sequence; and (iii) a protein binding sequence, e.g., an exterior protein binding sequence, and wherein the nucleic acid construct is a single-stranded DNA; and wherein the nucleic acid construct is circular, integrates at a frequency of less than about 0.001%, 0.005%, 0.01%, 0.05%, 0.1%, 0.5%, 1%, 1.5%, or 2% of the genetic element that enters the cell, and/or integrates into the genome of a target cell at less than 1, 2, 3, 4, 5, 6, 7, 8, 9, 10, 15, 20, 25, or 30 copies per genome In some embodiments, a genetic element encoding an effector (e.g., an exogenous or endogenous effector, e.g., as described herein) is codon optimized. In some embodiments, the genetic element is circular. In some embodiments, the genetic element is linear. In some embodiments, the genetic element comprises an anellovector, e.g., as described herein. In some embodiments, a genetic element described herein comprises one or more modified nucleotides (e.g., a base modification, sugar modification, or backbone modification). In some embodiments, the genetic element comprises a sequence encoding an ORF1 molecule (e.g., an Anellovirus ORF1 protein, e.g., as described herein). In some embodiments, the genetic element comprises a sequence encoding an ORF2 molecule (e.g., an Anellovirus ORF2 protein, e.g., as described herein). In some embodiments, the genetic element comprises a sequence encoding an ORF3 molecule (e.g., an Anellovirus ORF3 protein, e.g., as described herein).

In an aspect, the invention features a host cell or helper cell comprising: (a) a nucleic acid comprising a sequence encoding one or more of an ORF1 molecule, an ORF2 molecule, or an ORF3 molecule (e.g, a sequence encoding an Anellovirus ORF1 polypeptide described herein), wherein the nucleic acid is a plasmid, is a viral nucleic acid, or is integrated into a helper cell chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (ii) a protein binding sequence that binds the polypeptide of (a), wherein optionally the genetic element does not encode an ORF1 polypeptide (e.g., an ORF1 protein). For example, the host cell or helper cell comprises (a) and (b) either in cis (both part of the same nucleic acid molecule) or in trans (each part of a different nucleic acid molecule). In embodiments, the genetic element of (b) is circular, single-stranded DNA. In some embodiments, the host cell is a manufacturing cell line. In some embodiments, the host cell or helper cell is adherent or in suspension, or both. In some embodiments, the host cell or helper cell is grown in a microcarrier. In some embodiments, the host cell or helper cell is compatible with cGMP manufacturing practices. In some embodiments, the host cell or helper cell is grown in a medium suitable for promoting cell growth. In certain embodiments, once the host cell or helper cell has grown sufficiently (e.g., to an appropriate cell density), the medium may be exchanged with a medium suitable for production of anellovectors by the host cell or helper cell.

In an aspect, the invention features a pharmaceutical composition comprising an anellovector (e.g., a synthetic anellovector) as described herein. In embodiments, the pharmaceutical composition further comprises a pharmaceutically acceptable carrier or excipient. In embodiments, the pharmaceutical composition comprises a unit dose comprising about 10⁵-10¹⁴ genome equivalents of the anellovector per kilogram of a target subject. In some embodiments, the pharmaceutical composition comprising the preparation will be stable over an acceptable period of time and temperature, and/or be compatible with the desired route of administration and/or any devices this route of administration will require, e.g., needles or syringes. In some embodiments, the pharmaceutical composition is formulated for administration as a single dose or multiple doses. In some embodiments, the pharmaceutical composition is formulated at the site of administration, e.g., by a healthcare professional. In some embodiments, the pharmaceutical composition comprises a desired concentration of anellovector genomes or genomic equivalents (e.g., as defined by number of genomes per volume).

In an aspect, the invention features a method of treating a disease or disorder in a subject, the method comprising administering to the subject an anellovector, e.g., a synthetic anellovector, e.g., as described herein.

In an aspect, the invention features a method of delivering an effector or payload (e.g., an endogenous or exogenous effector) to a cell, tissue or subject, the method comprising administering to the subject an anellovector, e.g., a synthetic anellovector, e.g., as described herein, wherein the anellovector comprises a nucleic acid sequence encoding the effector. In embodiments, the payload is a nucleic acid.

In embodiments, the payload is a polypeptide.

In an aspect, the invention features a method of delivering an anellovector to a cell, comprising contacting the anellovector, e.g., a synthetic anellovector, e.g., as described herein, with a cell, e.g., a eukaryotic cell, e.g., a mammalian cell, e.g., in vivo or ex vivo.

In an aspect, the invention features a method of treating a disease or disorder in a subject, the method comprising administering to the subject an anelloVLP, e.g., a synthetic anelloVLP, e.g., as described herein.

In an aspect, the invention features a method of delivering an effector or payload (e.g., an endogenous or exogenous effector) to a cell, tissue or subject, the method comprising administering to the subject an anelloVLP, e.g., a synthetic anelloVLP, e.g., as described herein, wherein the anelloVLP comprises the effector (e.g., wherein the proteinaceous exterior of the anelloVLP encapsulates the effector). In embodiments, the payload is a nucleic acid. In embodiments, the payload is a polypeptide (e.g., a protein).

In an aspect, the invention features a method of delivering an anelloVLP to a cell, comprising contacting the anelloVLP, e.g., a synthetic anelloVLP, e.g., as described herein, with a cell, e.g., a eukaryotic cell, e.g., a mammalian cell, e.g., in vivo or ex vivo.

In an aspect, the invention features a method of making an anellovector, e.g., a synthetic anellovector. The method includes:
a) providing a host cell comprising:
(i) a first nucleic acid molecule comprising the nucleic acid sequence of a genetic element of an anellovector, e.g., a synthetic anellovector, as described herein, and
(ii) the first nucleic acid or a second nucleic acid molecule encoding one or more of an amino acid sequence chosen from ORF1, ORF2, ORF2/2, ORF2/3, ORF1/1, or ORF1/2, e.g., as listed in any one of Tables A1-A25, or an amino acid sequence having at least 70% (e.g., at least 70%, 75%, 80%, 85%, 90%, 95%, 96%, 97%, 98%, 99%, or 100%) sequence identity thereto; and
b) incubating the host cell under conditions suitable to make the anellovector.

In some embodiments, the method further includes, prior to step (a), introducing the first nucleic acid molecule and/or the second nucleic acid molecule into the host cell. In some embodiments, the second nucleic acid molecule is introduced into the host cell prior to, concurrently with, or after the first nucleic acid molecule. In other embodiments, the second nucleic acid molecule is integrated into the genome of the host cell. In some embodiments, the second nucleic acid molecule is a helper (e.g., a helper plasmid or the genome of a helper virus).

In another aspect, the invention features a method of manufacturing an anellovector composition, comprising:
a) providing a host cell comprising, e.g., expressing one or more components (e.g., all of the components) of an anellovector, e.g., a synthetic anellovector, e.g., as described herein. For example, the host cell comprises (a) a nucleic acid comprising a sequence encoding an Anellovirus ORF1 polypeptide described herein, wherein the nucleic acid is a plasmid, is a viral nucleic acid, or is integrated into a helper cell chromosome; and (b) a genetic element, wherein the genetic element comprises (i) a promoter element operably linked to a nucleic acid sequence (e.g., a DNA sequence) encoding an effector (e.g., an exogenous effector or an endogenous effector) and (i) a protein binding sequence (e.g, packaging sequence) that binds the polypeptide of (a), wherein the host cell or helper cell comprises (a) and (b) either in cis or in trans. In embodiments, the genetic element of (b) is circular, single-stranded DNA. In some embodiments, the host cell is a manufacturing cell line;
b) culturing the host cell under conditions suitable for producing a preparation of anellovectors from the host cell, wherein the anellovectors of the preparation comprise a proteinaceous exterior (e.g, comprising an ORF1 molecule) encapsulating the genetic element (e.g., as described herein), thereby making a preparation of anellovectors; and optionally, c) formulating the preparation of anellovectors, e.g., as a pharmaceutical composition suitable for administration to a subject.

In some embodiments, the components of the anellovector are introduced into the host cell at the time of production (e.g., by transient transfection). In some embodiments, the host cell stably expresses the components of the anellovector (e.g., wherein one or more nucleic acids encoding the components of the anellovector are introduced into the host cell, or a progenitor thereof, e.g., by stable transfection).

In some embodiments, the method further comprises one or more purification steps (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

In an aspect, the invention features a method of manufacturing an anellovector composition, comprising: a) providing a plurality of anellovectors described herein, or a preparation of anellovectors described herein; and b) formulating the anellovectors or preparation thereof, e.g., as a pharmaceutical composition suitable for administration to a subject.

In an aspect, the invention features a method of manufacturing an anelloVLP composition, comprising: a) providing a plurality of anelloVLPs described herein, or a preparation of anelloVLPs described herein; and b) formulating the anelloVLPs or preparation thereof, e.g., as a pharmaceutical composition suitable for administration to a subject.

In an aspect, the invention features a method of making a host cell, e.g., a first host cell or a producer cell (e.g., as shown in FIG. 20), e.g., a population of first host cells, comprising an anellovector, the method comprising introducing a genetic element, e.g., as described herein, to a host cell and culturing the host cell under conditions suitable for production of the anellovector. In some embodiments, the method further comprises introducing a helper, e.g., a helper virus, to the host cell. In some embodiments, the introducing comprises transfection (e.g., chemical transfection) or electroporation of the host cell with the anellovector.

In an aspect, the invention features a method of making an anellovector, comprising providing a host cell, e.g., a first host cell or producer cell (e.g., as shown in FIG. 20), comprising an anellovector, e.g., as described herein, and purifying the anellovector from the host cell. In some embodiments, the method further comprises, prior to the providing step, contacting the host cell with an anellovector, e.g., as described herein, and incubating the host cell under conditions suitable for production of the anellovector. In some embodiments, the host cell is the first host cell or producer cell described in the above method of making a host cell. In some embodiments, purifying the anellovector from the host cell comprises lysing the host cell.

In some embodiments, the method further comprises a second step of contacting the anellovector produced by the first host cell or producer cell with a second host cell, e.g., a permissive cell (e.g., as shown in FIG. 20), e.g., a population of second host cells. In some embodiments, the method further comprises incubating the second host cell inder conditions suitable for production of the anellovector. In some embodiments, the method further comprises purifying an anellovector from the second host cell, e.g., thereby producing an anellovector seed population. In some embodiments, at least about 2-100-fold more of the anellovector is produced from the population of second host cells than from the population of first host cells. In some embodiments, purifying the anellovector from the second host cell comprises lysing the second host cell. In some embodiments, the method further comprises a second step of contacting the anellovector produced by the second host cell with a third host cell, e.g., permissive cells (e.g., as shown in FIG. 20), e.g., a population of third host cells. In some embodiments, the method further comprises incubating the third host cell inder conditions suitable for production of the anellovector. In some embodiments, the method further comprises purifying an anellovector from the third host cell, e.g., thereby producing an anellovector stock population. In some embodiments, purifying the anellovector from the third host cell comprises lysing the third host cell. In some embodiments, at least about 2-100-fold more of the anellovector is produced from the population of third host cells than from the population of second host cells.

In some embodiments, the host cell is grown in a medium suitable for promoting cell growth. In certain embodiments, once the host cell has grown sufficiently (e.g., to an appropriate cell density), the medium may be exchanged with a medium suitable for production of anellovectors by the host cell. In some embodiments, anellovectors produced by a host cell separated from the host cell (e.g., by lysing the host cell) prior to contact with a second host cell. In some embodiments, anellovectors produced by a host cell are contacted with a second host cell without an intervening purification step.

In an aspect, the invention features a method of making a pharmaceutical anellovector preparation. The method comprises (a) making an anellovector preparation as described herein, (b) evaluating the preparation (e.g., a pharmaceutical anellovector preparation, anellovector seed population or the anellovector stock population) for one or more pharmaceutical quality control parameters, e.g., identity, purity, titer, potency (e.g., in genomic equivalents per anellovector particle), and/or the nucleic acid sequence, e.g., from the genetic element comprised by the anellovector, and (c) formulating the preparation for pharmaceutical use of the evaluation meets a predetermined criterion, e.g, meets a pharmaceutical specification. In some embodiments, evaluating identity comprises evaluating (e.g., confirming) the sequence of the genetic element of the anellovector, e.g., the sequence encoding the effector. In some embodiments, evaluating purity comprises evaluating the amount of an impurity, e.g., mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted anellovectors (e.g., an anellovector other than the desired anellovector, e.g., a synthetic anellovector as described herein), free viral capsid protein, adventitious agents, and aggregates. In some embodiments, evaluating titer comprises evaluating the ratio of functional versus non-functional (e.g., infectious vs non-infectious) anellovectors in the preparation (e.g., as evaluated by HPLC). In some embodiments, evaluating potency comprises evaluating the level of anellovector function (e.g., expression and/or function of an effector encoded therein or genomic equivalents) detectable in the preparation. In some embodiments, the impurities comprise residual denaturant (e.g., urea) or cellular substituents (e.g., proteasomes or ferritin).

In some embodiments, the formulated preparation is substantially free of pathogens, host cell contaminants or impurities; has a predetermined level of non-infectious particles or a predetermined ratio of particles:infectious units (e.g., <300:1, <200:1, <100:1, or <50:1). In some embodiments, multiple anellovectors can be produced in a single batch. In some embodiments, the levels of the anellovectors produced in the batch can be evaluated (e.g., individually or together).

In an aspect, the invention features a method of making a pharmaceutical anelloVLP preparation.

The method comprises (a) making an anelloVLP preparation as described herein, (b) evaluating the preparation (e.g., a pharmaceutical anelloVLP preparation, anelloVLP seed population or the anelloVLP stock population) for one or more pharmaceutical quality control parameters, e.g., identity, purity, titer, potency, and (c) formulating the preparation for pharmaceutical use of the evaluation meets a predetermined criterion, e.g, meets a pharmaceutical specification. In some embodiments, evaluating purity comprises evaluating the amount of an impurity, e.g., mycoplasma, endotoxin, host cell nucleic acids (e.g., host cell DNA and/or host cell RNA), animal-derived process impurities (e.g., serum albumin or trypsin), replication-competent agents (RCA), e.g., replication-competent virus or unwanted VLPs (e.g., an anelloVLP other than the desired anelloVLP, e.g., a synthetic anelloVLP as described herein), free viral capsid protein, adventitious agents, and aggregates. In some embodiments, evalating titer comprises evaluating the ratio of functional versus non-functional (e.g., infectious vs non-infectious) anelloVLPs in the preparation (e.g., as evaluated by HPLC). In some embodiments, evaluating potency comprises evaluating the level of anelloVLP function (e.g., expression and/or function of an effector encoded therein or genomic equivalents) detectable in the preparation. In some embodiments, the impurities comprise residual denaturant (e.g., urea) or cellular substituents (e.g., proteasomes or ferritin).

In some embodiments, the formulated preparation is substantially free of pathogens, host cell contaminants or impurities; has a predetermined level of non-infectious particles or a predetermined ratio of particles:infectious units (e.g., <300:1, <200:1, <100:1, or <50:1). In some embodiments, multiple anelloVLPs can be produced in a single batch. In some embodiments, the levels of the anelloVLPs produced in the batch can be evaluated (e.g., individually or together).

In an aspect, the invention features a host cell comprising:
(i) a first nucleic acid molecule comprising the nucleic acid sequence of a genetic element of an anellovector as described herein, and
(ii) optionally, a second nucleic acid molecule encoding one or more of an amino acid sequence chosen from ORF1, ORF2, ORF2/2, ORF2/3, ORF1/1, or ORF1/2 as listed in any one of Tables A1-A25, or an amino acid sequence having at least about 70% (e.g., at least about 70, 80, 90, 95, 96, 97, 98, 99, or 100%) sequence identity thereto.

In an aspect, the invention features a reaction mixture comprising an anellovector described herein and a helper virus, wherein the helper virus comprises a polynucleotide, e.g., a polynucleotide encoding an exterior protein, (e.g., an exterior protein capable of binding to the exterior protein binding sequence and, optionally, a lipid envelope), a polynucleotide encoding a replication protein (e.g., a polymerase), or any combination thereof.

In some embodiments, an anellovector (e.g., a synthetic anellovector) is isolated, e.g., isolated from a host cell and/or isolated from other constituents in a solution (e.g., a supernatant). In some embodiments, an anellovector (e.g., a synthetic anellovector) is purified, e.g., from a solution (e.g., a supernatant). In some embodiments, an anellovector is enriched in a solution relative to other constituents in the solution.

In some embodiments of any of the aforesaid anellovectors, compositions or methods, providing an anellovector comprises separating (e.g., harvesting) an anellovector from a composition comprising an anellovector-producing cell, e.g., as described herein. In other embodiments, providing an anellovector comprises obtaining an anellovector or a preparation thereof, e.g., from a third party.

In some embodiments of any of the aforesaid anellovectors, anellovectors, compositions or methods, the genetic element comprises an anellovector genome, e.g., as identified according to the method described in Example 9. In embodiments, the anellovector genome is an anellovector genome capable of self-replication and/or self-amplification. In some embodiments, the anellovector genome is not capable of self-replication and/or self-amplification. In some embodiments, the anellovector genome is capable of replicating and/or being amplified in trans, e.g., in the presence of a helper, e.g., a helper virus.

Additional features of any of the aforesaid anellovectors, anelloVLPs, compositions or methods include one or more of the following enumerated embodiments.

Those skilled in the art will recognize, or be able to ascertain using no more than routine experimentation, many equivalents to the specific embodiments of the invention described herein. Such equivalents are intended to be encompassed by the following enumerated embodiments.

### Enumerated Embodiments

1. A particle comprising:
a proteinaceous exterior comprising about 40-80 (e.g., about 60), 100-140 (e.g., about 120), or 160-200 (e.g., about 180) copies of an Anellovirus ORF1 molecule,
wherein the particle:
   (i) does not comprise (e.g., does not enclose) a polynucleotide (e.g., as determined using a nuclease protection assay as described herein),
   (ii) does not comprise (e.g., does not enclose) a polynucleotide of greater than 1000, 500, 200, or 100 nucleotides in length, or
   (iii) comprises less than about 1, 2, 3,4, 5, 6, 7, 8, 9, 10, 15, 20, 25, 30, 40, 50, 60, 70, 80, 90, 100, 150, 200, 250, 300, 400, 500, 600, 700, 800, 900, or 1000 nucleotides.

2. The particle of embodiment 1, wherein the Anellovirus ORF1 molecule comprises an ORF1 domain and an exogenous surface moiety.

3. The particle of embodiment 1, wherein the Anellovirus ORF1 molecule is bound to an exogenous surface moiety via a noncovalent integration or a covalent bond other than a peptide bond.

4. The particle of embodiment 1, wherein the Anellovirus ORF1 molecule does not comprise an arginine-rich domain.

5. The particle of embodiment 1, wherein the particle is a virus-like particle (VLP).

6. A particle comprising:
(a) a proteinaceous exterior comprising about 40-80 (e.g., about 60), 100-140 (e.g., about 120), or 160-200 (e.g., about 180) copies of an Anellovirus ORF1 molecule and an exogenous surface moiety, and
(b) a genetic element comprising a heterologous nucleic acid sequence encoding an exogenous effector.

7. A particle comprising:
a proteinaceous exterior comprising an Anellovirus ORF1 molecule, wherein the ORF1 molecule comprises an ORF1 domain and an exogenous surface moiety;
wherein one or more of:
a) the exogenous surface moiety is chosen from a receptor, a ligand, an antibody molecule (e.g., scFv), an antigen (e.g., a viral antigen, a bacterial antigen, a fungal antigen, or a parasite antigen) an adjuvant (e.g., TLR agonist, e.g., bacterial flagellin);
b) wherein the ORF1 molecule comprises a hypervariable region (HVR);
c) wherein the particle comprises a genetic element that encodes a peptide or polypeptide that boosts an immune response (e.g. an adjuvant, a TCR agonist (e.g., a bacterial flagellin));
d) wherein the exogenos surface moiety is between 1-5, 5-10, 10-20, 20-50, 50-100, 100-200, 200-500, or 500-1000 amino acids in length;
e) wherein a polypeptide linker region is situated between the exogenous surface moiety and the ORF1 molecule,
f) wherein the particle comprises 1-2, 2-5, 5-10, 10-20, 20-40, 40-60, 60-80, 80-100, 100-125, 125-150, 150-175, 175-200, 200-225, 225-250, 250-275, or 275-300 copies of the exogenous surface moiety;
g) wherein the proteinaceous exterior comprises (i) a plurality of ORF1 molecules lacking the exogenous surface moiety (e.g., a wild-type ORF1 molecule) and (ii) a plurality of ORF1 molecules that comprise the exogenous surface moiety, wherein optionally the ratio of (i) (ii) is between 10:1-5:1, 5:1-2:1, 2:1-1:2, 1:2-1:5, or 1:5-1:10; and/or
h) wherein the particle further comprises a second exogenous surface moiety.

8. The particle of embodiment 7, wherein the exogenous surface moiety is situated at an insertion point between an N-terminal portion of the ORF1 domain and a C-terminal portion of the ORF1 domain.

9. The particle of embodiment 8, wherein the insertion point is in the HVR.

10. The particle of any of embodiments 7-9, further comprising a genetic element comprising a heterologous nucleic acid sequence encoding an exogenous effector.

11. The particle of any of embodiments embodiments 7-10, wherein the particle does not comprise (e.g., does not enclose) a polynucleotide, or does not comprise (e.g., does not enclose) a polynucleotide of greater than 1000, 500, 200, or 100 nucleotides in length, or comprises less than about 1, 2, 3,4, 5, 6, 7, 8, 9, 10, 15, 20, 25, 30, 40, 50, 60, 70, 80, 90, 100, 150, 200, 250, 300, 400, 500, 600, 700, 800, 900, or 1000 nucleotides.

12. A particle comprising:
a proteinaceous exterior comprising an Anellovirus ORF1 molecule, and an exogenous surface moiety, wherein the exogenous surface moiety is covalently bound to the ORF1 molecule using a bond other than a peptide bond.

13. The particle of embodiment 12, wherein the exogenous surface moiety is attached to an NHS moiety, and the exogenous surface moiety is bound to the ORF1 molecule via the NHS moiety.

14. The particle of embodiment 12 or 13, wherein a non-polypeptide linker is situated between the exogenous surface moiety and the ORF1 molecule.

15. The particle of embodiment 14, wherein the non-polypeptide linker comprises a click linkage.

16. The particle of embodiment 14, wherein the non-polypeptide linker is produced by a click reaction between a DBCO moiety and an azide moiety.

17. The particle of embodiment 16, wherein, prior to the click reaction, the DBCO moiety is attached to the Anellovirus ORF1 molecule via an NHS moiety.

18. The particle of embodiment 16 or 17, wherein, prior to the click reaction, the azide moiety is attached to the exogenous surface moiety via an NHS moiety.

19. The particle of embodiment 17 or 18, wherein the NHS moiety of the DBCO moiety is attached to a lysine residue on the surface of the Anellovirus ORF1 molecule.

20. The particle of any of embodiments 17-19, wherein the NHS moiety of the azide moiety is attached to a lysine residue on the surface of the exogenous surface moiety.

21. The particle of embodiment 16, wherein, prior to the click reaction, the DBCO moiety is attached to the exogenous surface moiety via an NHS moiety.

22. The particle of embodiment 16 or 21, wherein, prior to the click reaction, the azide moiety is attached to the Anellovirus ORF1 molecule via an NHS moiety.

23. The particle of embodiment 21 or 22, wherein the NHS moiety of the DBCO moiety is attached to a lysine residue on the surface of the exogenous surface moiety.

24. The particle of any of embodiments 21-23, wherein the NHS moiety of the azide moiety is attached to a lysine residue on the surface of the Anellovirus ORF1 molecule.

25. A particle comprising:
a proteinaceous exterior comprising an Anellovirus ORF1 molecule, and an exogenous surface moiety, wherein the exogenous surface moiety is non-covalently bound to the ORF1 molecule.

26. The particle of embodiment 25, wherein the ORF1 molecule comprises an exogenous binding domain (e.g., MS2 coat protein or avidin), and the exogenous surface moiety comprises a cognate binding moiety (e.g., MS2 hairpin or biotin) that binds the exogenous binding domain.

27. The particle of any of embodiments 12-26, wherein the exogenous surface moiety comprises a polypeptide.

28. The particle of any of embodiments 12-27, wherein the exogenous surface moiety comprises a small molecule or nucleic acid molecule (e.g., polynucleotide).

29. The particle of any of the preceding, wherein the ratio of ORF1 molecule to exogenous surface moiety is between about 60:1-30:1, 30:1-20:1, 20:1-10:1, or 10:1-1:1.

30. The particle of any of the preceding embodiments, wherein the antibody molecule is a bispecific antibody molecule.

31. The particle of embodiment 30, wherein the bispecific antibody molecule comprises:
a first antigen-binding domain that binds a first antigen on a first type of host cell, and
a second antigen-binding domain that binds a second antigen on a second type of host cell.

32. The particle of any of the preceding embodiments, which is capable of entering a target cell, e.g., by endocytosis.

33. The particle of embodiment 32, wherein the exogenous surface moiety binds to a cognate moiety in the target cell.

34. The particle of embodiment 32, wherein the particle comprises a genetic element encoding an exogenous effector to be delivered to the interior of the target cell.

35. The particle of any of the preceding embodiments, wherein a genetic element is enclosed within the proteinaceous exterior.

36. The particle of any of the preceding embodiments, which particle does not comprise a polynucleotide, or does not comprise a polynucleotide of greater than 1000, 500, 200, or 100 nucleotides in length.

37. The particle of any of the preceding embodiments, wherein the Anellovirus ORF1 molecule comprises:
(b) a first region comprising an Anellovirus ORF1 jelly-roll region, e.g., an amino acid sequence having at least 30% (e.g., at least about 30, 35, 40, 50, 60, 70, 80, 90, 95, 96, 97, 98, 99, or 100%) sequence identity to a Anellovirus ORF1 jelly-roll region sequence described herein or a sequence comprising at least 6 beta strands;
(c) a second region comprising an Anellovirus ORF1 N22 domain, e.g., an amino acid sequence having at least 30% (e.g., at least about 30, 35, 40, 50, 60, 70, 80, 90, 95, 96, 97, 98, 99, or 100%) sequence identity to an Anellovirus ORF1 N22 domain sequence described herein; and
(d) a third region comprising an Anellovirus ORF1 C-terminal domain (CTD), e.g., an amino acid sequence having at least 30% (e.g., at least about 30, 35, 40, 50, 60, 70, 80, 90, 95, 96, 97, 98, 99, or 100%) sequence identity to an Anellovirus ORF1 CTD sequence described herein; and
wherein the Anellovirus ORF1 molecule does not comprise an Anellovirus ORF1 arginine-rich region, e.g., a sequence of at least about 40 amino acids comprising at least 60%, 70%, or 80% basic residues (e.g., arginine, lysine, or a combination thereof).

38. A preparation comprising the particle of any of the preceding embodiments.

39. The preparation of embodiment 38, wherein the preparation comprises less than 10¹⁰-10¹⁴ (e.g., less than 10¹⁰-10¹¹, 10¹¹-10¹², 10¹²-10¹³, or 10¹³-10¹⁴) viral genome equivalents of nucleic acid molecules (e.g., genetic elements, e.g., of an anellovector as described herein) per kilogram of a subject to be administered the composition (e.g., as determined by qPCR or by measuring optical density).

40. A polypeptide, e.g., an Anellovirus ORF1 molecule, comprising:
(b) a first region comprising an Anellovirus ORF1 jelly-roll region, e.g., an amino acid sequence having at least 30% (e.g., at least about 30, 35, 40, 50, 60, 70, 80, 90, 95, 96, 97, 98, 99, or 100%) sequence identity to a Anellovirus ORF1 jelly-roll region sequence described herein or a sequence comprising at least 6 beta strands;
(c) a second region comprising an Anellovirus ORF1 N22 domain, e.g., an amino acid sequence having at least 30% (e.g., at least about 30, 35, 40, 50, 60, 70, 80, 90, 95, 96, 97, 98, 99, or 100%) sequence identity to an Anellovirus ORF1 N22 domain sequence described herein; and
(d) a third region comprising an Anellovirus ORF1 C-terminal domain (CTD), e.g., an amino acid sequence having at least 30% (e.g., at least about 30, 35, 40, 50, 60, 70, 80, 90, 95, 96, 97, 98, 99, or 100%) sequence identity to an Anellovirus ORF1 CTD sequence described herein; and
wherein the polypeptide does not comprise an Anellovirus ORF1 arginine-rich region, e.g., a sequence of at least about 40 amino acids comprising at least 60%, 70%, or 80% basic residues (e.g., arginine, lysine, or a combination thereof).

41. A nucleic acid molecule encoding a polypeptide of embodiment 40.

42. A particle comprising a proteinaceous exterior comprising an Anellovirus ORF1 molecule, wherein the ORF1 molecule comprises an ORF1 domain and an exogenous surface domain;
wherein the particle is made by contacting a plurality of Anellovirus ORF1 molecules in a cell-free solution under conditions suitable to form a proteinaceous exterior comprising the plurality of Anellovirus ORF1 molecules.

43. The particle of embodiment 42, wherein the particle does not comprise (e.g., does not enclose) a polynucleotide, or does not comprise (e.g., does not enclose) a polynucleotide of greater than 1000, 500, 200, or 100 nucleotides in length.

44. A method of making a particle, the method comprising:
contacting a plurality of Anellovirus ORF1 molecules in a cell-free solution under conditions suitable to form a proteinaceous exterior comprising the plurality of Anellovirus ORF1 molecules;
thereby making a particle.

45. A method of modulating a biological activity in a cell, the method comprising:
contacting the cell with a particle of any of the proceeding embodiments; wherein the cell comprises a moiety on its surface that binds to the exogenous surface moiety of the particle.

46. A method of targeting a particle to a cell, the method comprising:
contacting the cell with a particle of any of the proceeding embodiments;
wherein the cell comprises a moiety on its surface that binds to the exogenous surface moiety of the particle.

47. The polypeptide, particle, nucleic acid molecule, or method of any of the preceding embodiments, wherein the exogenous surface moiety is fused to the N-terminus of the Anellovirus ORF1 molecule.

48. The polypeptide, particle, nucleic acid molecule, or method of any of the preceding embodiments, wherein the exogenous surface moiety is fused to the C-terminus of the Anellovirus ORF1 molecule.

49. The polypeptide, particle, nucleic acid molecule, or method of any of the preceding embodiments, wherein the exogenous surface moiety is inserted within the amino acid sequence of the Anellovirus ORF1 molecule.

50. An ORF1 molecule comprising an exogenous surface moiety, wherein the exogenous surface moiety is fused to, replaces, and/or is situated within an insertion point of an ORF1 domain (e.g., within an HVR or P2).

51. An ORF1 molecule comprising an exogenous surface moiety, wherein the exogenous surface moiety is fused to, replaces, and/or is situated at an insertion point between residues corresponding to positions 284-285 of Ring 10 ORF1, e.g., in an ORF1 domain (e.g., within the HVR).

52. An ORF1 molecule comprising an exogenous surface moiety, wherein the exogenous surface moiety is fused to, replaces, and/or is situated at an insertion point between residues corresponding to positions 328-329 of Ring 10 ORF1, e.g., in an ORF1 domain (e.g., within the HVR).

53. An ORF1 molecule comprising an exogenous surface moiety, wherein the exogenous surface moiety is fused to, replaces, and/or is situated at an insertion point between residues corresponding to positions 256-383 of Ring 10 ORF1, e.g., in an ORF1 domain (e.g., within the HVR).

54. An ORF1 molecule comprising an exogenous surface moiety, wherein the exogenous surface moiety is fused to, replaces, and/or is situated at an insertion point between residues corresponding to positions 251-383 of Ring 10 ORF1, e.g., in an ORF1 domain (e.g., within the HVR).

55. An ORF1 molecule comprising an exogenous surface moiety, wherein the exogenous surface moiety is fused to, replaces, and/or is situated at an insertion point between residues corresponding to positions 251-384 of Ring 10 ORF1, e.g., in an ORF1 domain (e.g., within the HVR).

56. An ORF1 molecule comprising an exogenous surface moiety, wherein the exogenous surface moiety is attached to (e.g., conjugated to) the amino acid residue (e.g., a cysteine residue) corresponding to position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of Ring 10 ORF1, e.g., in an ORF1 domain (e.g., within the HVR).

57. The ORF1 molecule of any of embodiments 51-56, wherein the exogenous surface moiety forms a pentamer when the ORF1 molecule is complexed with four other ORF1 molecules (e.g., four other copies of the ORF1 molecule).

58. The ORF1 molecule of any of embodiments 51-56, wherein the exogenous surface moiety forms a trimer when the ORF1 molecule is complexed with four other ORF1 molecules (e.g., four other copies of the ORF1 molecule).

59. The ORF1 molecule of any of embodiments 51-56, wherein the exogenous surface moiety forms a dimer when the ORF1 molecule is complexed with four other ORF1 molecules (e.g., four other copies of the ORF1 molecule).

60. A protein complex comprising five ORF1 molecules, wherein each of the ORF1 molecules comprises:
(i) an ORF1 domain, and
(ii) an exogenous surface moiety;
   wherein the exogenous surface moieties of the five ORF1 molecules forms a pentamer.

61. The protein complex of embodiment 60, wherein each of the exogenous surface moieties is fused to, replaces, and/or is situated within an insertion point of an ORF1 domain (e.g., within an HVR) of the corresponding ORF1 molecule.

62. The protein complex of embodiment 60, wherein each of the exogenous surface moieties is fused to, replaces, and/or is situated within an insertion point between residues corresponding to positions 284-285 in an ORF1 domain (e.g., within the HVR) of Ring 10 ORF1. 63. The protein complex of embodiment 60, wherein each of the exogenous surface moieties is fused to, replaces, and/or is situated within an insertion point between residues corresponding to positions 328-329 in an ORF1 domain (e.g., within the HVR) of Ring 10 ORF1.

64. The protein complex of embodiment 60, wherein each of the exogenous surface moieties is fused to, replaces, and/or is situated within an insertion point between residues corresponding to positions 256-383 in an ORF1 domain (e.g., within the HVR) of Ring 10 ORF1.

65. The protein complex of embodiment 60, wherein each of the exogenous surface moieties is fused to, replaces, and/or is situated within an insertion point between residues corresponding to positions 251-383 in an ORF1 domain (e.g., within the HVR) of Ring 10 ORF1.

66. The protein complex of embodiment 60, wherein each of the exogenous surface moieties is fused to, replaces, and/or is situated within an insertion point between residues corresponding to positions 251-384 in an ORF1 domain (e.g., within the HVR) of Ring 10 ORF1.

67. The protein complex of embodiment 60, wherein each of the exogenous surface moieties is attached to (e.g., conjugated to) the amino acid residue (e.g., a cysteine residue) corresponding to position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of Ring 10 ORF1, e.g., in an ORF1 domain (e.g., within the HVR).

68. The protein complex of any of embodiments 60-67, wherein the exogenous surface moieties of the ORF1 molecules have the same amino acid sequences.

69. The protein complex of any of embodiments 60-67, wherein at least two (e.g., at least 2, 3, 4, or 5) of the exogenous surface moieties of the ORF1 molecules have different amino acid sequences.

70. A protein complex comprising three ORF1 molecules, wherein each of the ORF1 molecules comprises:
(i) an ORF1 domain, and
(ii) an exogenous surface moiety;
wherein the exogenous surface moieties of the three ORF1 molecules forms a trimer.

71. The protein complex of embodiment 70, wherein each of the exogenous surface moieties is fused to, replaces, and/or is situated within an insertion point of an ORF1 domain (e.g., within an HVR) of the corresponding ORF1 molecule.

72. The protein complex of embodiment 70, wherein each of the exogenous surface moieties is fused to, replaces, and/or is situated within an insertion point between positions 284-285 in an ORF1 domain (e.g., within the HVR) of the corresponding ORF1 molecule.

73. The protein complex of embodiment 70, wherein each of the exogenous surface moieties is fused to, replaces, and/or is situated within an insertion point between positions 328-329 in an ORF1 domain (e.g., within the HVR) of the corresponding ORF1 molecule.

74. The protein complex of embodiment 70, wherein each of the exogenous surface moieties is fused to, replaces, and/or is situated within an insertion point between positions 256-383 in an ORF1 domain (e.g., within the HVR) of the corresponding ORF1 molecule.

75. The protein complex of embodiment 70, wherein each of the exogenous surface moieties is fused to, replaces, and/or is situated within an insertion point between positions 251-383 in an ORF1 domain (e.g., within the HVR) of the corresponding ORF1 molecule.

76. The protein complex of embodiment 70, wherein each of the exogenous surface moieties is fused to, replaces, and/or is situated within an insertion point between positions 251-384 in an ORF1 domain (e.g., within the HVR) of the corresponding ORF1 molecule.

77. The protein complex of embodiment 70, wherein each of the exogenous surface moieties is attached to (e.g., conjugated to) the amino acid residue (e.g., a cysteine residue) at position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of the corresponding ORF1 molecule, e.g., in an ORF1 domain (e.g., within the HVR).

78. The protein complex of any of embodiments 70-77, wherein the exogenous surface moieties of the ORF1 molecules have the same amino acid sequences.

79. The protein complex of any of embodiments 70-78, wherein at least two (e.g., at least 2 or 3) of the exogenous surface moieties of the ORF1 molecules have different amino acid sequences.

80. A protein complex comprising two ORF1 molecules, wherein each of the ORF1 molecules comprises:
(i) an ORF1 domain, and
(ii) an exogenous surface moiety;
wherein the exogenous surface moieties of the two ORF1 molecules forms a dimer.

81. The protein complex of embodiment 80, wherein each of the exogenous surface moieties is fused to, replaces, and/or is situated within an insertion point of an ORF1 domain (e.g., within an HVR) of the corresponding ORF1 molecule.

82. The protein complex of embodiment 80, wherein each of the exogenous surface moieties is fused to, replaces, and/or is situated within an insertion point between residues corresponding to positions 284-285 in an ORF1 domain (e.g., within the HVR) of Ring 10 ORF1.

83. The protein complex of embodiment 80, wherein each of the exogenous surface moieties is fused to, replaces, and/or is situated within an insertion point between residues corresponding to positions 328-329 in an ORF1 domain (e.g., within the HVR) of Ring 10 ORF1.

84. The protein complex of embodiment 80, wherein each of the exogenous surface moieties is fused to, replaces, and/or is situated within an insertion point between residues corresponding to positions 256-383 in an ORF1 domain (e.g., within the HVR) of Ring 10 ORF1.

85. The protein complex of embodiment 80, wherein each of the exogenous surface moieties is fused to, replaces, and/or is situated within an insertion point between residues corresponding to positions 251-383 in an ORF1 domain (e.g., within the HVR) of Ring 10 ORF1.

86. The protein complex of embodiment 80, wherein each of the exogenous surface moieties is fused to, replaces, and/or is situated within an insertion point between residues corresponding to positions 251-384 in an ORF1 domain (e.g., within the HVR) of Ring 10 ORF1.

87. The protein complex of embodiment 80, wherein each of the exogenous surface moieties is attached to (e.g., conjugated to) the amino acid residue (e.g., a cysteine residue) corresponding to position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of Ring 10 ORF1, e.g., in an ORF1 domain (e.g., within the HVR).

88. The protein complex of any of embodiments 80-87, wherein the exogenous surface moieties of the two ORF1 molecules have the same amino acid sequences.

89. The protein complex of any of embodiments 80-87, wherein the exogenous surface moieties of the two ORF1 molecules have different amino acid sequences.

90. The polypeptide, particle, nucleic acid molecule, method, or protein complex of any of the preceding embodiments, wherein the polypeptide or ORF1 molecule comprises one or more substitutions of a cysteine residues (e.g., one or more cysteine to alanine substitutions or one or more cysteine to serine substitutions).

91. The polypeptide, particle, nucleic acid molecule, method, or protein complex of any of the preceding embodiments, wherein the polypeptide or ORF1 molecule comprises a cysteine to serine mutation at one or more (e.g., 1, 2, 3, 4, 5, 6, 7, or 8) residues corresponding to position 63, 70, 137, 269, 403, 460, 503, and/or 515 of a Ring 10 ORF1 protein (e.g., as described herein).

92. The polypeptide, particle, nucleic acid molecule, method, or protein complex of any of the preceding embodiments, wherein the polypeptide or ORF1 molecule comprises a cysteine to alanine mutation at one or more (e.g., 1, 2, 3, 4, 5, 6, or 7) residues corresponding to position 63, 137, 269, 403, 460, 503, and/or 515 of a Ring 10 ORF1 protein (e.g., as described herein).

93. The polypeptide, particle, nucleic acid molecule, method, or protein complex of any of the preceding embodiments, wherein the polypeptide or ORF1 molecule comprises a cysteine to serine mutation at the residue corresponding to position 70 of a Ring 10 ORF1 protein (e.g., as described herein).

94. The polypeptide, particle, nucleic acid molecule, method, or protein complex of any of the preceding embodiments, wherein the polypeptide or ORF1 molecule comprises a substitution of an amino acid residue (e.g., a threonine, serine, asparagine, alanine, glutamine, or lysine residue) to cysteine.

95. The polypeptide, particle, nucleic acid molecule, method, or protein complex of embodiment 94, wherein the polypeptide or ORF1 molecule comprises a threonine to cysteine substitution, e.g., at the position corresponding to position 365 of a Ring 10 ORF1 protein (e.g., as described herein).

96. The polypeptide, particle, nucleic acid molecule, method, or protein complex of embodiment 94, wherein the polypeptide or ORF1 molecule comprises a serine to cysteine substitution, e.g., at the position corresponding to position 284 of a Ring 10 ORF1 protein (e.g., as described herein).

97. The polypeptide, particle, nucleic acid molecule, method, or protein complex of embodiment 94, wherein the polypeptide or ORF1 molecule comprises an asparagine to cysteine substitution, e.g., at the position corresponding to position 290 of a Ring 10 ORF1 protein (e.g., as described herein).

98. The polypeptide, particle, nucleic acid molecule, method, or protein complex of embodiment 94, wherein the polypeptide or ORF1 molecule comprises a lysine to cysteine substitution, e.g., at the position corresponding to position 317 of a Ring 10 ORF1 protein (e.g., as described herein).

99. The polypeptide, particle, nucleic acid molecule, method, or protein complex of embodiment 94, wherein the polypeptide or ORF1 molecule comprises a lysine to cysteine substitution, e.g., at the position corresponding to position 324 of a Ring 10 ORF1 protein (e.g., as described herein).

100. The polypeptide, particle, nucleic acid molecule, method, or protein complex of embodiment 94, wherein the polypeptide or ORF1 molecule comprises an alanine to cysteine substitution, e.g., at the position corresponding to position 362 of a Ring 10 ORF1 protein (e.g., as described herein).

101. The polypeptide, particle, nucleic acid molecule, method, or protein complex of embodiment 94, wherein the polypeptide or ORF1 molecule comprises a serine to cysteine substitution, e.g., at the position corresponding to position 363 of a Ring 10 ORF1 protein (e.g., as described herein).

102. The polypeptide, particle, nucleic acid molecule, method, or protein complex of embodiment 94, wherein the polypeptide or ORF1 molecule comprises an asparagine to cysteine substitution, e.g., at the position corresponding to position 369 of a Ring 10 ORF1 protein (e.g., as described herein).

103. The polypeptide, particle, nucleic acid molecule, method, or protein complex of embodiment 94, wherein the polypeptide or ORF1 molecule comprises a lysine to cysteine substitution, e.g., at the position corresponding to position 371 of a Ring 10 ORF1 protein (e.g., as described herein).

104. The polypeptide, particle, nucleic acid molecule, method, or protein complex of embodiment 94, wherein the polypeptide or ORF1 molecule comprises a glutamine to cysteine substitution, e.g., at the position corresponding to position 287 of a Ring 10 ORF1 protein (e.g., as described herein).

105. The polypeptide, particle, nucleic acid molecule, method, or protein complex of embodiment 94, wherein the polypeptide or ORF1 molecule comprises one or more substitutions to cysteine at one or more positions corresponding to Y254, R263, N264, K265, L272, G273, T274, R276, H283, T285, N288, D291, Q308, D311, W312, T313, E314, D316, H318, N319, T321, T328, K329, T341, Q343, T354, Q358, T361, T364, Q368, D374, P376, P378, Y380, and/or 1381 of a Ring 10 ORF1 protein (e.g., as described herein).

106. A particle comprising:
(a) a proteinaceous exterior comprising an ORF1 molecule; and
(b) a genetic element comprising a heterologous nucleic acid sequence encoding an exogenous effector;
wherein the genetic element is enclosed within the proteinaceous exterior; and
wherein the particle has one or more of the following characteristics:
   (i) the genetic element (e.g., a DNA genetic element) does not comprise an Anellovirus 5′ UTR or an origin of replication;
   (ii) the sequence encoding the exogenous effector takes up at least 90%, 95%, 96%, 97%, 98%, 99% or 100% of the genetic element (e.g., a DNA genetic element);
   (iii) the heterologous nucleic acid sequence takes up at least 90%, 95%, 96%, 97%, 98%, 99% or 100% of the genetic element (e.g., a DNA genetic element);
   (iv) the particle does not comprise a detectable amount of (e.g., any) polypeptides from a host cell, or comprises less than 5, 10, 15, 20, 25, 30, 40, or 50 copies of a polypeptide from a host cell;
   (v) the particle does not comprise a detectable amount of (e.g., any) nucleic acid molecules from a host cell, or comprises less than 2, 3, 4, or 5 copies of a nucleic acid molecule from a host cell;
   (vi) the particle comprises a denaturant in a concentration of less than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M;
   (vii) does not substantially replicate when introduced into a cell (e.g., a human cell); and/or
   (viii) has a symmetrical morphology.

107. The particle of embodiment 106, wherein the heterologous nucleic acid sequence is about 60-100, 100-200, 200-300, 300-400, 400-500, 500-600, 600-700, 700-800, 800-900, 900-1000, 1000-1500, or 1500-2000, 2000-3000, 3000-4000, or 4000-5000 nucleotides in length.

108. A population of the particles of embodiment 106, wherein at least 75%, 80%, 90%, 95%, 96%, 97%, 98%, 99%, or 100% of the particles in the population comprise at least 50, 55, or 60 copies of an Anellovirus ORF1 molecule.

109. A population of the particles of embodiment 106, wherein at least 90% of the particles in the population have a diameter of at least 30, 31, 32, 33, 34, or 35 nm.

110. A population of the particles of embodiment 106, wherein at least 90%, 95%, 96%, 97%, 98%, 99%, or 100% of the particles have a symmetrical morphology.

111. A population of the particles of embodiment 106, wherein the population does not comprise a detectable amount of polypeptides from a host cell, or comprises less than 1, 2, 3, 4, 5, 10, 15, 20, 25, 30, 40, or 50 copies per particle of a polypeptide from a host cell.

112. A population of the particles of embodiment 106, wherein the population does not comprise a detectable amount of nucleic acid molecules from a host cell, or comprises less than 1, 2, 3, 4, 5, 10, 15, 20, 25, 30, 40, or 50 copies per particle of a nucleic acid molecule from a host cell.

113. A population of the particles of embodiment 106, wherein the population comprises less than 10 ng of nucleic acids.

114. A population of the particles of embodiment 106, wherein the population does not comprise a detectable amount of nucleic acid molecules from a host cell, or comprises less than 1, 2, 3, 4, 5, 10, 15, 20, 25, 30, 40, or 50 copies per particle of a nucleic acid molecule having a length of 200 bp or less from a host cell.

115. A particle comprising:
a proteinaceous exterior comprising about 40-80 (e.g., about 60) copies of an ORF1 molecule; and
wherein the particle:
(i) does not comprise (e.g., does not enclose) a polynucleotide,
(ii) does not comprise (e.g., does not enclose) detectable levels of polynucleotides, (iii) does not comprise (e.g., does not enclose) a polynucleotide of greater than 1000, 500, 200, or 100 nucleotides in length,
(iv) does not comprise (e.g., does not enclose) a polynucleotide comprising any contiguous nucleic acid sequences of at least 50, 100, 150, 200, 300, 400, 500, 600, 700, 800, 900, or 1000 nucleotides in length having least 75%, 80%, 85%, 90%, 95%, 96%, 97%, 98%, 99%, or 100% sequence identity to contiguous sequence in a wild-type Anellovirus genome (e.g., as described herein), and/or
(v) does not comprise a polynucleotide comprising an Anellovirus 5′ UTR or an origin of replication.

116. The particle of embodiment 115, further comprising an exogenous effector.

117. The particle of embodiment 116, wherein the exogenous effector is enclosed within the proteinaceous exterior.

118. The particle of embodiment 115 or 116, wherein the exogenous effector is a polypeptide.

119. The particle of any of embodiments 115-118, wherein the exogenous effector is a small molecule.

120. A composition comprising a plurality of particles, the particles comprising a proteinaceous exterior comprising about 40-80 (e.g., about 60) copies of an ORF1 molecule;
wherein at least 50%, 60%, 70%, 75%, 80%, 85%, 90%, 95%, 96%, 97%, 98%, 99%, 99.5%, or 99.9% of the particles do not comprise (e.g., do not enclose):
   (i) a polynucleotide,
   (ii) a nucleic acid molecule of greater than 1000, 500, 200, or 100 nucleotides in length,
   (iii) a plurality of polynucleotides,
   (iv) a circular nucleic acid molecule,
   (v) a single-stranded nucleic acid molecule, and/or
   (vi) a genetic element (e.g., a genetic element of an anellovector), e.g., as described herein; or
wherein the composition comprises less than 10¹⁰-10¹⁴ (e.g., less than 10¹⁰-10¹¹, 10¹²-10¹², 10¹²-10¹³, or 10¹³-10¹⁴) viral genome equivalents of nucleic acid molecules (e.g., genetic elements, e.g., of an anellovector as described herein) per kilogram of a subject to be administered the composition (e.g., as determined by qPCR or by measuring optical density).

121. The composition of embodiment 120, further comprising a denaturant (e.g., urea), e.g., in concentration of less than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M; proteasomes; or ferritin.

122. The composition of embodiment 120 or 121, wherein the composition comprises 0.01-100 mg of the particles (e.g., 0.01-1, 1-10, 10-20, 20-30, 30-40, 40-50, 50-60, 60-70, 70-80, 80-90, or 90-100 mg of the particles).

123. A method of disassembling a particle, the method comprising:
(a) providing a mixture comprising a particle and a denaturant, wherein the particle comprises:
   (i) a proteinaceous exterior comprising a plurality of Anellovirus ORF1 molecules, and
   (ii) a nucleic acid molecule (e.g., a nucleic acid endogenous to a host cell or a nucleic acid exogenous to a host cell, e.g., an anellovirus genome); and
(b) incubating the mixture under conditions suitable for:
   disassembly of the proteinaceous exterior, and
   dissociation of the nucleic acid molecule from the proteinaceous exterior.

124. The method of embodiment 123, wherein the Anellovirus ORF1 molecules were made in mammalian cells.

125. The method of embodiment 123 or 124, wherein the conditions suitable for disassembly of the proteinaceous exterior comprises one or more of: a predetermined conductivity, a detergent (e.g., SDS (e.g., 0.1% SDS), Tween, or Triton), a chaotropic agent (e.g, urea), a high salt solution (e.g., a solution comprising NaCl, e.g., at a concentration of at least about 1M, e.g., at least about 0.5, 0.6, 0.7, 0.8, 0.9, 1.0, 1.1, 1.2, 1.3, 1.4, 1.5, 2, 3, 4, or 5M), or conditions involving a predetermined temperature.

126. The method of any of embodiments 123-125, wherein the mixture comprises a population of the particles.

127. The method of any of embodiments 123-126, wherein the incubating of (b) results in at least 50%, 60% . . . 95%, or 100% of the population of particles being disassembled.

128. The method of any of embodiments 123-127, further comprising a step of (c) removing (partially or completely) the nucleic acid molecule from the mixture, e.g., by washing.

129. The method of any of embodiments 123-128, wherein the host cell is a human cell.

130. A method of making an anellovector, the method comprising:
(a) providing a mixture comprising a plurality of Anellovirus ORF1 molecules, wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(b) subjecting the mixture to conditions suitable for in vitro assembly of the Anellovirus ORF1 molecules; and
(c) incubating the Anellovirus ORF1 molecules with a plurality of genetic elements, under conditions suitable for assembly of the Anellovirus ORF1 molecules into one or more anellovectors each enclosing one or more of the genetic elements.

131. The method of embodiment 130, wherein the mixture provided in (a) is under denaturing conditions, e.g., wherein the mixture comprises a denaturant at a level sufficient to disassemble a complex (e.g., a proteinaceous exterior) comprising at least about 20, 30, 40, 50, or 60 copies, or 20-30, 30-40, 40-50, or 50-60 copies, of the Anellovirus ORF1 molecule.

132. The method of embodiment 130 or 131, wherein the conditions suitable for in vitro assembly comprise reducing the concentration of a denaturant or removing the mixture from denaturing conditions.

133. A method of making an anellovector, the method comprising:
(a) providing a mixture comprising a plurality of Anellovirus ORF1 molecules and subjecting the mixture to denaturing conditions (e.g., providing a denaturant as part of the mixture, e.g., contacting the mixture with a denaturant), wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(b) subjecting the mixture to non-denaturing conditions (e.g., reducing the concentration of the denaturant to a level) suitable for in vitro assembly of the Anellovirus ORF1 molecules (e.g., by dialysis); and
(c) incubating the Anellovirus ORF1 molecules with a plurality of genetic elements, under conditions suitable for assembly of the Anellovirus ORF1 molecules into one or more anellovectors each enclosing one or more of the genetic elements.

134. The method of embodiment 133, wherein (b) and (c) are performed concurrently.

135. The method of embodiment 133, wherein (b) is performed prior to (c).

136. The method of any of embodiments 133-135, wherein the genetic elements are introduced into a mixture comprising the Anellovirus ORF1 molecules prior to, concurrently with, or after (b).

137. The method of any of embodiments 133-136, wherein at least 75%, 80%, 90%, 95%, 96%, 97%, 98%, 99%, or 100% of the plurality of ORF1 molecules in the mixture of (a) are comprised in capsomers (e.g., decamers or particles of 25-40 nm in diameter, e.g., 25-30, 30-32, 32-35, or 35-40 nm in diameter or about 25, 26, 27, 28, 29, 30, 31, 32, 33, 34, 35, 36, 37, 38, 39, or 40 nm).

138. The method of any of embodiments 133-137, wherein the ratio of ORF1 molecules in the mixture of (a) comprised in capsomers (e.g., decamers) compared to ORF1 molecules in the mixture of (a) comprised in particles is at least 2:1, 3:1, 4:1, 5:1, 10:1, 50:1, 100:1, 500:1, 1000:1, 5000:1, or 10,000:1.

139. The method of any of embodiments 133-138, wherein at least 75%, 80%, 90%, 95%, 96%, 97%, 98%, 99%, or 100% of the plurality of ORF1 molecules after the incubating of (c) are comprised in anellovectors (e.g., 60mers or particles of at least 30, 31, 32, 33, 34, or 35 nm in diameter).

140. The method of any of embodiments 133-139, wherein the genetic element encodes an exogenous effector.

141. The method of any of embodiments 133-140, wherein the genetic element is an oligonucleotide.

142. The method of any of embodiments 133-141, wherein the genetic element does not encode a polypeptide or functional nucleic acid.

143. The method of any of embodiments 133-142, wherein the concentration of the denaturant after step (b) is no more than about 0.01M, 0.1M, 0.2M, 0.3M, 0.4M, 0.5M, 0.6M, 0.7M, 0.8M, 0.9M,

1M, 1.1M, 1.2M, 1.3M, 1.5M, 1.5M, 1.6M, 1.7M, 1.8M, 1.9M, or 2M.

144. The method of any of embodiments 133-143, wherein, after the incubating of (c), at least 75%, 80%, 90%, 95%, 96%, 97%, 98%, 99%, or 100% of the particles in the mixture comprise at least 50, 55, or 60 copies of an Anellovirus ORF1 molecule.

145. The method of any of embodiments 133-144, wherein, after the incubating of (c), at least 90% of the particles in the mixture have a diameter of at least 30, 31, 32, 33, 34, or 35 nm.

146. The method of any of embodiments 133-145, wherein, after the incubating of (c), at least 90%, 95%, 96%, 97%, 98%, 99%, or 100% of the particles in the mixture have a symmetrical morphology.

147. The method of any of embodiments 133-146, wherein the denaturant is selected from a chaotropic agent (e.g., urea), heat (e.g., temperature above about 85, 86, 87, 88, 89, 90, 91, 92, 93, 94, or 95° C.), or pH (e.g., acidic pH or basic pH).

148. A method of making an anelloVLP, the method comprising:
(a) providing a mixture comprising a plurality of Anellovirus ORF1 molecules, wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(b) subjecting the mixture to conditions suitable for in vitro assembly of the Anellovirus ORF1 molecules; and
(c) incubating the Anellovirus ORF1 molecules with a plurality of effectors (e.g., exogenous effectors), under conditions suitable for assembly of the Anellovirus ORF1 molecules into one or more anelloVLPs each enclosing one or more of the effectors.

149. The method of embodiment 148, wherein the mixture provided in (a) is under denaturing conditions, e.g., wherein the mixture comprises a denaturant at a level sufficient to disassemble a complex (e.g., a proteinaceous exterior) comprising at least about 20, 30, 40, 50, or 60 copies, or 20-30, 30-40, 40-50, or 50-60 copies, of the Anellovirus ORF1 molecule.

150. The method of embodiment 148 or 149, wherein the conditions suitable for in vitro assembly comprise reducing the concentration of a denaturant or removing the mixture from denaturing conditions.

151. A method of making an anelloVLP, the method comprising:
(a) providing a mixture comprising a plurality of Anellovirus ORF1 molecules and a denaturant, wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(b) reducing the concentration of the denaturant to a level suitable for in vitro assembly of the Anellovirus ORF1 molecules; and
(c) incubating the Anellovirus ORF1 molecules with a plurality of effectors (e.g., exogenous effectors), under conditions suitable for in vitro assembly of the Anellovirus ORF1 molecules into one or more anelloVLPs each enclosing one or more of the effectors.

152. The method of embodiment 151, wherein the effectors are introduced into a mixture comprising the Anellovirus ORF1 molecules prior to, concurrently with, or after (b).

153. The method of embodiment 151 or 152, wherein at least 75%, 80%, 90%, 95%, 96%, 97%, 98%, 99%, or 100% of the plurality of ORF1 molecules in the mixture of (a) are comprised in capsomers (e.g., decamers or particles of at most 25-40 nm in diameter, e.g., 25-30, 30-32, 32-35, or 35-40 nm in diameter or about 25, 26, 27, 28, 29, 30, 31, 32, 33, 34, 35, 36, 37, 38, 39, or 40 nm).

154. The method of any of embodiments 151-153, wherein the ratio of ORF1 molecules in the mixture of (a) comprised in capsomers (e.g., decamers) compared to ORF1 molecules in the mixture of (a) comprised in particles is at least 2:1, 3:1, 4:1, 5:1, 10:1, 50:1, 100:1, 500:1, 1000:1, 5000:1, or 10,000:1.

155. The method of any of embodiments 151-154, wherein at least 75%, 80%, 90%, 95%, 96%, 97%, 98%, 99%, or 100% of the plurality of ORF1 molecules after the incubating of (c) are comprised in anelloVLPs (e.g., 60mers or particles of at least 30, 31, 32, 33, 34, or 35 nm in diameter).

156. The method of any of embodiments 151-155, wherein the anelloVLP has one or more of the following characteristics:
(i) does not comprise (e.g., does not enclose) a polynucleotide,
(ii) does not comprise (e.g., does not enclose) detectable levels of polynucleotides,
(iii) does not comprise (e.g., does not enclose) a polynucleotide of greater than 1000, 500, 200, or 100 nucleotides in length,
(iv) does not comprise (e.g., does not enclose) a polynucleotide comprising any contiguous nucleic acid sequences of at least 50, 100, 150, 200, 300, 400, 500, 600, 700, 800, 900, or 1000 nucleotides in length having least 75%, 80%, 85%, 90%, 95%, 96%, 97%, 98%, 99%, or 100% sequence identity to contiguous sequence in a wild-type Anellovirus genome (e.g., as described herein), and/or
(v) does not comprise a polynucleotide comprising an Anellovirus 5′ UTR or an origin of replication.

157. The method of any of embodiments 151-156, wherein the concentration of the denaturant after step (b) is no more than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M.

158. The method of any of embodiments 151-157, wherein, after the incubating of (c), at least 75%, 80%, 90%, 95%, 96%, 97%, 98%, 99%, or 100% of the particles in the mixture comprise at least 50, 55, or 60 copies of an Anellovirus ORF1 molecule.

159. The method of any of embodiments 151-158, wherein, after the incubating of (c), at least 90% of the particles in the mixture have a diameter of at least 30, 31, 32, 33, 34, or 35 nm.

160. The method of any of embodiments 151-159, wherein, after the incubating of (c), at least 90%, 95%, 96%, 97%, 98%, 99%, or 100% of the particles in the mixture have a symmetrical morphology.

161. The method of any of embodiments 151-160, wherein the denaturant is selected from a chaotropic agent (e.g., urea), heat (e.g., temperature above about 85, 86, 87, 88, 89, 90, 91, 92, 93, 94, or 95° C.), or pH (e.g., acidic pH or basic pH).

162. A method of making an anelloVLP, the method comprising:
(a) providing a mixture comprising a particle and a denaturant, wherein the particle comprises:
   (i) a proteinaceous exterior comprising a plurality of Anellovirus ORF1 molecules, and
   (ii) a nucleic acid molecule (e.g., a host cell nucleic acid molecule); and
(b) incubating the mixture under conditions suitable for:
   disassembly of the proteinaceous exterior, and
   dissociation of the nucleic acid molecule from the proteinaceous exterior;
(c) providing a mixture comprising a plurality of Anellovirus ORF1 molecules and a denaturant, wherein at least 75%, 80%, 90%, 95%, 96%, 97%, 98%, 99%, or 100% of the plurality of ORF1 molecules are not comprised in a particle comprising about 40-80 (e.g., about 60) copies of an ORF1 molecule;
(d) reducing the concentration of the denaturant to a level suitable for in vitro assembly of the Anellovirus ORF1 molecules; and
(e) incubating the Anellovirus ORF1 molecules with a plurality of effectors (e.g., exogenous effectors), under conditions suitable for assembly of the Anellovirus ORF1 molecules into one or more anelloVLPs each enclosing one or more of the effectors.

163. A polypeptide, e.g., an ORF1 molecule, comprising one or more of:
(a) a first region comprising an amino acid sequence having at least 70% (e.g., at least about 70, 80, 90, 95, 96, 97, 98, 99, or 100%) sequence identity to an arginine-rich region sequence described herein (e.g., MPYYYRRRRYNYRRPRWYGRGWIRRPFRRRFRRKRRVR (SEQ ID NO: 216) or MAWGWWKRRRRWWFRKRWTRGRLRRRWPRSARRRPRRRRVRRRRRWRRGRRKTRTYRRRR RFRRRGRK (SEQ ID NO: 186), or as listed in any one of Tables A1-A25) or a sequence of at least about 40 amino acids comprising at least 60%, 70%, or 80% basic residues (e.g., arginine, lysine, or a combination thereof),
(b) a second region comprising an amino acid sequence having at least 30% (e.g., at least about

30, 35, 40, 50, 60, 70, 80, 90, 95, 96, 97, 98, 99, or 100%) sequence identity to a jelly-roll region sequence described herein (e.g., PTYTTIPLKQWQPPYKRTCYIKGQDCLIYYSNLRLGMNSTMYEKSIVPVHWPGGGSFSVSMLTLD ALYDIHKLCRNWWTSTNQDLPLVRYKGCKITFYQSTFTDYIVRIHTELPANSNKLTYPNTHPLM MMMSKYKHIIPSRQTRRKKKPYTKIFVKPPPQFENKWYFATDLYKIPLLQIHCTACNLQNPFVKP DKLSNNVTLWSLNT (SEQ ID NO: 217), or as listed in any of any one of Tables A1-A25) or a sequence comprising at least 6 (e.g., at least 6, 7, 8, 9, 10, 11, or 12) beta strands;
(c) a third region comprising an amino acid sequence having at least 30% (e.g., at least about 30, 35, 40, 50, 60, 70, 80, 90, 95, 96, 97, 98, 99, or 100%) sequence identity to an N22 domain sequence described herein (e.g.,
   TMALTPFNEPIFTQIQYNPDRDTGEDTQLYLLSNATGTGWDPPGIPELILEGFPLWLIYWGFADFQ KNLKKVTNIDTNYMLVAKTKFTQKPGTFYLVILNDTFVEGNSPYEKQPLPEDNIKWYPQVQYQL EAQNKLLQTGPFTPNIQGQLSDNISMFYKFYFK (SEQ ID NO: 219), or as listed in any of any one of Tables A1-A25); and
(d) a fourth region comprising an amino acid sequence having at least 30% (e.g., at least about 30, 35, 40, 50, 60, 70, 80, 90, 95, 96, 97, 98, 99, or 100%) sequence identity to an Anellovirus ORF1 C-terminal domain (CTD) sequence described herein (e.g.,
   WGGSPPKAINVENPAHQIQYPIPRNEHETTSLQSPGEAPESILYSFDYRHGNYTTTALSRISQDWA LKDTVSKITEPDRQQLLKQALECLQISEETQEKKEKEVQQLISNLRQQQQLYRERIISLLKDQ (SEQ ID NO: 220), or as listed in any of any one of Tables A1-A25);
wherein the ORF1 molecule comprises at least one difference (e.g., a mutation, chemical modification, or epigenetic alteration) relative to a wild-type ORF1 protein (e.g., as described herein), e.g., an insertion, substitution, chemical or enzymatic modification, and/or deletion, e.g., a deletion of a domain (e.g., one or more of an arginine-rich region, jelly-roll domain, HVR, N22, or CTD, e.g., as described herein).

164. The polypeptide of embodiment 163, wherein the amino acid sequences of the region of (a), (b), (c), and (d) have at least 90% sequence identity to their respective references.

165. The polypeptide of embodiment 163, wherein the polypeptide comprises:
(i) the first region and the second region;
(ii) the first region and the third region;
(iii) the first region and the fourth region;
(iv) the second region and the third region;
(v) the second region and the fourth region;
(vi) the third region and the fourth region;
(vii) the first region, the second region, and the third region;
(viii) the first region, the second region, and the fourth region;
(ix) the first region, the third region, and the fourth region; or
(x) the second region, the third region, and the fourth region.

166. The polypeptide of any of embodiments 163-165, wherein:
the first region comprises an amino acid sequence having at least 70% (e.g., at least about 70, 80, 90, 95, 96, 97, 98, 99, or 100%) sequence identity to an arginine-rich region sequence as listed in any one of Tables A1-A25;
the second region comprises an amino acid sequence having at least 70% (e.g., at least about 70, 80, 90, 95, 96, 97, 98, 99, or 100%) sequence identity to a jelly-roll region sequence as listed in any one of Tables A1-A25;
the third region comprises an amino acid sequence having at least 70% (e.g., at least about 70, 80, 90, 95, 96, 97, 98, 99, or 100%) sequence identity to an N22 domain sequence as listed in any one of Tables A1-A25; and/or
the fourth region comprises an amino acid sequence having at least 70% (e.g., at least about 70, 80, 90, 95, 96, 97, 98, 99, or 100%) sequence identity to a CTD sequence as listed in any one of Tables A1-A25.

167. The polypeptide according to embodiment 166, wherein the amino acid sequences of the first, second, third and fourth region have at least 90% sequence identity to their respective references.

168. The polypeptide of any of the preceding embodiments, wherein the polypeptide comprises, in N-terminal to C-terminal order, the first region, the second region, the third region, and the fourth region.

169. The polypeptide of any of the preceding embodiments, wherein the at least one difference comprises at least one difference in the first region relative to the arginine-rich region of a wild-type ORF1 protein.

170. The polypeptide of any of the preceding embodiments, wherein the first region comprises an arginine-rich region from the ORF1 protein of an Anellovirus other than the wild-type Anellovirus to which the polypeptide, or the portion thereof excluding the first region, has greatest sequence identity.

171. The polypeptide of any of the preceding embodiments, wherein the first region comprises an amino acid sequence having at least 70% sequence identity to the arginine-rich region from an Anellovirus other than the wild-type Anellovirus to which the polypeptide has greatest sequence identity.

172. The polypeptide of any of the preceding embodiments, wherein the second region comprises a jelly-roll region from the ORF1 protein of an Anellovirus other than the wild-type Anellovirus to which the polypeptide, or the portion thereof excluding the second region, has greatest sequence identity.

173. The polypeptide of any of the preceding embodiments, wherein the second region comprises an amino acid sequence having at least 70% sequence identity to the jelly-roll region from an Anellovirus other than the wild-type Anellovirus to which the polypeptide has greatest sequence identity.

174. The polypeptide of any of the preceding embodiments, wherein the third region comprises an N22 domain from the ORF1 protein of an Anellovirus other than the wild-type Anellovirus to which the polypeptide, or the portion thereof excluding the third region, has greatest sequence identity.

175. The polypeptide of any of the preceding embodiments, wherein the third region comprises an amino acid sequence having at least 70% sequence identity to the N22 region from an Anellovirus other than the wild-type Anellovirus to which the polypeptide has greatest sequence identity.

176. The polypeptide of any of the preceding embodiments, wherein the fourth region comprises a CTD domain from the ORF1 protein of an Anellovirus other than the wild-type Anellovirus to which the polypeptide, or the portion thereof excluding the fourth region, has greatest sequence identity.

177. The polypeptide of any of the preceding embodiments, wherein the fourth region comprises an amino acid sequence having at least 70% sequence identity to the CTD region from an Anellovirus other than the wild-type Anellovirus to which the polypeptide has greatest sequence identity.

178. The polypeptide of any of embodiments 163-177, wherein the HVR sequence is positioned between the second region and the third region.

179. The polypeptide of embodiment 178, wherein the HVR sequence comprises an amino acid sequence having at least 70% (e.g., at least about 70, 80, 90, 95, 96, 97, 98, 99, or 100%) sequence identity to the HVR from an Anellovirus other than the wild-type Anellovirus to which the ORF1 protein has greatest sequence identity.

180. The polypeptide of any of embodiment 178 or 179, wherein the HVR sequence is heterologous relative to one or more of the first region, second region, third region, and/or fourth region.

181. The polypeptide of any of embodiments 178-180, wherein the HVR sequence comprises an HVR from the ORF1 protein of an Anellovirus other than the wild-type Anellovirus to which the polypeptide, or the portion thereof excluding the HVR sequence, has greatest sequence identity.

182. The polypeptide of any of embodiments 178-181, wherein the HVR sequence comprises an amino acid sequence having at least 70% sequence identity to the HVR from an Anellovirus other than the wild-type Anellovirus to which the polypeptide has greatest sequence identity.

183. The anellovector of any of the preceding embodiments, wherein the proteinaceous exterior comprises a polypeptide of any of embodiments 58-77. 184. The particle of any of the preceding embodiments, wherein the proteinaceous exterior comprises a polypeptide of any of embodiments 58-77.

185. The anelloVLP of any of the preceding embodiments, wherein the proteinaceous exterior comprises a polypeptide of any of embodiments 58-77.

186. A method of making two or more different Anellovirus ORF molecules, the method comprising:
(i) providing an insect cell comprising a nucleic acid construct encoding two or more different Anellovirus ORF molecules (e.g., two or more of an ORF1, ORF2, ORF2/2, ORF2/3, ORF1/1, and/or ORF1/2 molecule);
(ii) incubating the insect cell under conditions suitable for expression of the two or more different Anellovirus ORF molecules.

187. The method of embodiment 186, wherein the nucleic acid construct comprises sequences encoding all of an ORF1, ORF2, ORF2/2, ORF2/3, ORF1/1, and/or ORF1/2 molecule.

188. The method of embodiment 186, further comprising incubating the insect cell under conditions suitable for secretion of the Anellovirus ORF molecule.

189. The method of embodiment 186, further isolating the Anellovirus ORF molecule from the insect cell.

190. The method of embodiment 189, wherein the isolating step comprises lysing the insect cell.

191. The method of any of embodiments 186-190, wherein the Anellovirus ORF comprises an Anellovirus ORF1 molecule.

192. A method of making an Anellovirus ORF1 molecule, the method comprising:
(i) providing an insect cell comprising a nucleic acid construct encoding an Anellovirus ORF1 molecule, wherein:
   (a) the Anellovirus ORF1 molecule has a molecular weight of at least 101 kDa,
   (b) the Anellovirus ORF1 molecule is a full-length Anellovirus ORF1 protein,
   (c) a plurality of the Anellovirus ORF1 molecules, when in the presence of an Anellovirus genetic element, enclose the Anellovirus genetic element,
   (d) the Anellovirus ORF1 molecule is not a TTV ORF1 protein,
   (e) the Anellovirus ORF1 molecule is a Betatorquevirus or Gammatorquevirus ORF1 molecule; or
   (f) the Anellovirus ORF1 molecule comprises an Anellovirus ORF1 Arginine-rich region and an Anellovirus C-terminal domain;
(ii) incubating the insect cell under conditions suitable for expression of the Anellovirus ORF1 molecule.

193. The method of embodiment 192, further comprising incubating the insect cell under conditions suitable for secretion of the Anellovirus ORF1 molecule.

194. The method of embodiment 192, further isolating the Anellovirus ORF1 molecule from the insect cell.

195. The method of embodiment 194, wherein the isolating step comprises lysing the insect cell.

196. The method of any of the preceding embodiments, wherein the incubation step produces an amount of the Anellovirus ORF1 molecule detectable by Western blot, e.g., as described herein.

197. A method of making an Anellovirus ORF molecule (e.g., an ORF1, ORF2, ORF2/2, ORF2/3, ORF1/1, and/or ORF1/2 molecule), the method comprising:
(i) providing an insect cell (e.g., an Sf9 cell) comprising a nucleic acid construct encoding the Anellovirus ORF molecule;
(ii) incubating the insect cell under conditions suitable for expression of a plurality of the Anellovirus ORF molecules; and
(iii) optionally isolating, purifying, and/or enriching the plurality of Anellovirus ORF molecules from the insect cell or other components or constituents thereof;
thereby making the Anellovirus ORF molecule.

198. The method of embodiment 197, wherein the Anellovirus ORF molecule is fused to a marker (e.g., a His tag), e.g., at its N-terminal end or at its C-terminal end (e.g., as described in Table E1 and/or Example 9).

199. The method of embodiment 197 or 198, wherein the insect cell further comprises a nucleic acid construct encoding one or more additional Anellovirus ORF molecules (e.g., one or more of an ORF1, ORF2, ORF2/2, ORF2/3, ORF1/1, and/or ORF1/2 molecule), and wherein the method further comprises:
incubating the insect cell under conditions suitable for expression of a plurality of the one or more additional Anellovirus ORF molecules, e.g., prior to, concurrently with, or subsequent to step (ii); and
optionally isolating, purifying, and/or enriching the plurality of the one or more additional Anellovirus ORF molecules from the insect cell or other components or constituents thereof, e.g., prior to, concurrently with, or subsequent to step (iii).

200. The method of embodiment 199, wherein the nucleic acid construct encoding the one or more additional Anellovirus ORF molecules is the same as the nucleic acid construct of (i).

201. The method of embodiment 200, wherein the nucleic acid construct of (i) comprises sequences encoding 2, 3, 4, 5, or all 6 of an Anellovirus ORF1, ORF2, ORF2/2, ORF2/3, ORF1/1, and/or ORF1/2 molecule.

202. The method of embodiment 200, wherein the nucleic acid construct of (i) encodes an Anellovirus ORF1, ORF2, ORF2/2, ORF2/3, ORF1/1, and ORF1/2 molecule.

203. The method of embodiment 200, wherein the nucleic acid construct of (i) comprises the full open reading frame region of an Anellovirus genome.

204. The method of embodiment 199, wherein the nucleic acid construct encoding the one or more additional Anellovirus ORF molecules is different from the nucleic acid construct of (i).

205. The method of any of embodiments 199-204, wherein the Anellovirus ORF molecules are from the same Anellovirus genome.

206. The method of any of embodiments 199-204, wherein the Anellovirus ORF molecules are from a plurality of Anellovirus genomes (e.g., wherein the ORF1 molecule is from one Anellovirus genome and the ORF2 molecule is from a different Anellovirus genome).

207. The method of any of embodiments 199-206, wherein one or more of the Anellovirus ORF molecules are from an Alphatorquevirus (e.g., as listed in Table E2).

208. The method of any of embodiments 199-207, wherein one or more of the Anellovirus ORF molecules are from a Betatorquevirus (e.g., as listed in Table E2).

209. The method of any of embodiments 199-208, wherein one or more of the Anellovirus ORF molecules are from a Gammatorquevirus (e.g., as listed in Table E2).

210. The method of any of embodiments 199-209, wherein the nucleic acid construct or constructs each comprises a promoter (e.g., a promoter controlling expression of one or more of the Anellovirus ORF molecules, e.g., a baculovirus polyhedron promoter).

211. The method of any of embodiments 199-210, further comprising incubating the insect cell under conditions suitable for secretion of the Anellovirus ORF molecules.

212. The method of any of embodiments 199-211, wherein the isolating step comprises lysing the insect cell.

213. The method of any of embodiments 199-212, wherein the incubation step produces an amount of the Anellovirus ORF molecule (e.g., ORF1 molecule) detectable by Western blot, e.g., as described herein.

214. The method of any of emcodiments 199-213, wherein the incubation step produces at least 1, 2, 3, 4, 5, or 6 mg of the Anellovirus ORF1 molecule per 1 L of cell culture (e.g., Sf9 culture).

215. The method of any of the preceding embodiments, wherein the Anellovirus ORF molecules are isolated, purified, or enriched by isopycnic centrifugation.

216. The method of any of the preceding embodiments, wherein the Anellovirus ORF molecule is an Anellovirus ORF1 molecule, and wherein the method further comprises: contacting, in vitro, the isolated, purified, or enriched Anellovirus ORF1 molecule with a genetic element under conditions suitable for enclosure of the genetic element by a proteinaceous exterior comprising the Anellovirus ORF1 molecule, e.g., as described herein.

Other features, objects, and advantages of the invention will be apparent from the description and drawings, and from the claims.

Unless otherwise defined, all technical and scientific terms used herein have the same meaning as commonly understood by one of ordinary skill in the art to which this invention belongs. All publications, patent applications, patents, and other references mentioned herein are incorporated by reference in their entirety. In addition, the materials, methods, and examples are illustrative only and not intended to be limiting.

### BRIEF DESCRIPTION OF THE DRAWINGS

The following detailed description of the embodiments of the invention will be better understood when read in conjunction with the appended drawings. For the purpose of illustrating the invention, there are shown in the drawings embodiments that are presently exemplified. It should be understood, however, that the invention is not limited to the precise arrangement and instrumentalities of the embodiments shown in the drawings. The patent or application file contains at least one drawing executed in color. Copies of this patent or patent application publication with color drawing(s) will be provided by the Office upon request and payment of the necessary fee.

FIG. 1 is a series of diagrams showing production of exemplary malaria peptide conjugation constructs comprising the C-terminal region of a CS protein.

FIG. 2 is a series of diagrams showing the structure of an exemplary malaria peptide conjugation construct on the surface of an anellovector capsid.

FIG. 3 is a diagram showing separation of in vitro circularized DNA into positive and negative sense circularized single-stranded DNA (ssDNA) after denaturation.

FIGS. 4A and 4B are a series of diagrams showing successful expression of Anellovirus ORF1 molecules in cells. (A) Anellovirus strains for which ORF1 expression was detected from Sf9 cells included Ring2, Ring3, Ring4, Ring5, Ring6, Ring9, and Ring10 (e.g., as described herein). (B) Virus-like particles (VLPs) were observed by electron microscopy after production of Anellovirus ORF1 proteins for Ring2 and Ring10, as well as for chicken anemia virus (CAV) VPl.

FIGS. 5A-5C are a series of diagrams showing successful expression and purification of Ring2 ORF1 and ORF2 proteins from Sf9 cells. (A) Exemplary workflow for purification of ORF1 and ORF2 proteins from Sf9 cells. (B) Western blot using anti-ORF1 antibody, showing detection of Ring2 ORF1 proteins produced in Sf9 cells as described. (C) Electron micrograph showing formation of virus-like particles (VLPs) from Ring2 ORF1 proteins produced in Sf9 cells as described.

FIGS. 6A-6C are a series of diagrams showing disassembly of Ring2 VLPs using urea as a chaotropic denaturant. (A) Ring2 ORF1 protein was produced and purified from Sf9 cells as described, and VLPs were detected by electron microscopy. (B) After treatment with urea, VLPs are no longer observed, but instead the solution contains small ORF1 capsomers. (C) Expanded view of the boxed area shown in FIG. 6B, showing detail of the capsomers.

FIGS. 7A-7B are a series of electron micrographs showing exemplary symmetrical (A) and asymmetrical (B) particle morphologies.

FIG. 8 depicts expression of Ring2 ORF1 with a C-terminal His tag in insect cells.

FIG. 9 depicts expression of Ring1 ORF1 and ORF1/1 with a C-terminal His tag in insect cells.

FIG. 10 depicts expression of Ring2 ORF1 with an N-terminal His-tag, with or without PreScission cleavage sequence, in insect cells.

FIG. 11 depicts expression of Ring1 ORFs 1/1, 1/2, 2, 2/2, and 2/3 as C-terminal His-tagged recombinant proteins in insect cells.

FIG. 12 depicts expression of individual Ring2 ORFs in insect cells. Two exposures of the same blot are shown in the middle and right panels. The left panel shows the structures of Ring2 constructs tested as indicated.

FIG. 13 depicts baculovirus-mediated co-expression of Ring2 ORF1+“FullORF”, ORF1+ORF2, ORF1+ORF2/2, and ORF1+ORF2/3 in insect cells.

FIG. 14 depicts simultaneous co-expression of multiple Ring2 proteins in insect cells using baculovirus.

FIG. 15 depicts expression of ORFs from Anellovirus genome delivered into insect cells by baculovirus and by transfection.

FIG. 16 shows that expression of Ring1 ORF2 is independent of the polyhedron promoter (arrow labeled pH) in Sf9 cells.

FIG. 17 depicts co-delivery of Ring2 ORF1-His and Ring2 genomic DNA into Sf9 cells, followed by incubation and fractionation on a CsCl linear density gradient. An anti-His tag Western blot of fractions is shown at the top of the figure, as well as a qPCR assay of each fraction. Bottom panels show transmission electron microscopy images of two individual fractions and a pool of fractions, as indicated by boxes on the Western blot. The inset in the middle panel is a zoomed-in view showing proteasome-like structures.

FIG. 18 depicts characterization of Sf9 isopycnic fractions by immunogold electron microscopy.

FIG. 19 depicts expression of ORF1 from additional Anellovirus strains.

FIG. 20 is a schematic showing an exemplary workflow for production of anellovectors (e.g., replication-competent or replication-deficient anellovectors as described herein).

FIG. 21 is a graph showing primer specificity for primer sets designed for quantification of TTV and TTMV genomic equivalents. Quantitative PCR based on SYBR green chemistry shows one distinct peak for each of the amplification products using TTMV or TTV specific primer sets, as indicated, on plasmids encoding the respective genomes.

FIG. 22 is a graph showing an exemplary amplification plot for linear amplification of TTMV (Target 1) or TTV (Target 2) over a 7 log 10 of genome equivalent concentrations. Genome equivalents were quantified over 7 10-fold dilutions with high PCR efficiencies and linearity (R² TTMV: 0.996; R² TTV: 0.997).

FIGS. 23A and 23B are a series of diagrams showing that a tandem Anellovirus plasmid can increase anellovirus or anellovector production. (A) Plasmid map for an exemplary tandem Anellovirus plasmid. (B) Transfection of HEK293T cells with a tandem Anellovirus plasmid resulted in production of four times the number of viral genomes compared to single-copy harboring plasmids.

FIG. 23C is a gel electrophoresis image showing circularization of TTMV-LY2 plasmids pVL46-063 and pVL46-240.

FIG. 23D is a chromatogram showing copy numbers for linear and circular TTMV-LY2 constructs, as determined by size exclusion chromatography (SEC).

FIG. 24 is a schematic showing the domains of an Anellovirus ORF1 molecule and the hypervariable region to be replaced with a hypervariable domain from a different Anellovirus.

FIG. 25 is a schematic showing the domains of ORF1 and the hypervariable region that will be replaced with a protein or peptide of interest (POI) from a non-anellovirus source.

FIG. 26 is a series of diagrams showing the design of an exemplary anellovector genetic element based on an Anellovirus genome. The protein-coding region was deleted from the anellovirus genome (left), leaving the anelloviral non-coding region (NCR), including the viral promoter, 5′UTR conserved domain (5CD), and GC-rich region. Payload DNA was inserted into the non-coding region at the protein-coding locus (right). The resulting anellovector harbored the payload DNA (including open reading frames, genes, non-coding RNAs, etc.) and the essential anellovirus cis replication and packaging elements, but lacked the essential protein elements for replication and packaging.

FIG. 27 is a diagram showing an alignment of 36-nucleotide GC-rich regions from nine Anellovirus genome sequences, and a consensus sequence based thereon (SEQ ID NOS 818-827, respectively, in order of appearance).

FIG. 28 is a series of diagrams showing ORF1 structures from Anellovirus strains LY2 and CBD203. Putative domains are labeled: arginine-rich region (arg-rich), core region comprising a jelly-roll domain, hypervariable region (HVR), N22 region, and C-terminal domain (CTD), as indicated.

FIG. 29 is a graph showing the ability of an in vitro circularized (IVC) TTV-tth8 genome (IVC TTV-tth8) compared to a TTV-tth8 genome in a plasmid to yield TTV-tth8 genome copies at the expected density in HEK293T cells.

FIG. 30 is a series of graphs showing the ability of an in vitro circularized (IVC) LY2 genome (WT LY2 IVC) and a wild-type LY2 genome in plasmid (WT LY2 Plasmid) to yield LY2 genome copies at the expected density in Jurkat cells.

FIG. 31A is a schematic presentation of full-length Ring 10 ORF1 (also referred to herein as Lyl) labeled and colored by domains. The structural arginine-rich motif (ARM) is shown in purple, the structural jelly roll (JR) domain is shown in red, the spike P1 domain is shown in blue, the spike P2 domain is shown in green, and the C-terminal domain is shown in cyan. Residue numbers beginning each domain and the structural C-terminal domain (also referred to as the C-terminus) are indicated above.

FIG. 31B is the sequence of full-length Ring 10 ORF1 colored as in FIG. 31A with residue numbers indicated above. In bold are residues included in the Ring 10 delARM construct including the initial residue, K46, which is labeled. A dashed line above the sequence indicates residues not observed in the density. Secondary structure elements are indicated above with β-strands as arrows and α-helices as zig-zag lines. The JR β-strands are labeled B-I per convention while additional secondary structures are numbered by their domain. Three peptides used to generate polyclonal antibodies are underlined.

FIG. 31C shows western blot analysis of Ring 10 delARM after expression (Expression) and after purification and storage (Purification). A molecular weight marker is labeled to the left of the gels, while arrows on the right indicate the band of Ring 10 delARM before (Ring 10 delARM) and after proteolysis (Ring 10 delARM Fragment). Polyclonal antibodies used to probe the western blots are indicated below and colored by the peptides used to generate them.

FIG. 31D is an overlay of the 3D reconstruction of Ring 10 delARM VLP electron density and 60-mer VLP molecular structure colored as in FIG. 31A. The spike P1 and P2 domains are labeled.

FIG. 31E depicts one ORF1 protomer, shown in its electron density with domains labeled and colored as in FIG. 31A.

FIG. 31F depicts the electron density of Ring 10 delARM VLP colored by its local resolution. The bar (left) indicates the resolution (unit in angstrom) scale by color. The particle (right) is oriented as in FIG. 31D.

FIG. 32A depicts 60 Ring 10 structural jelly roll (JR) domains with one uniquely colored in red. Sixty Ring 10 structural jelly roll (JR) domains form the core of anellovirus particles.

FIG. 32B depicts two JR domains, shown in red, with the observed C-terminal domain backbone colored in cyan. The JR domains are arbitrarily labeled JR¹ and JR² with the first (K48) and last (V562) observed residues for each protomer labeled with the corresponding number for clarity.

FIG. 32C depicts a single JR domain oriented to show the β-sheet on the interior of the particle core. Sidechains of basic residues in position to contact with the viral genome are shown and labeled.

FIG. 32D depicts the structural arginine-rich region, JR, and structural C-terminal domains of Ring 10 aligned with corresponding ORF1 sequences from different anellovirus genera (indicated in parentheses). Residues of Ring 10 are colored as in FIG. 31A-31D. Basic residues of Ring 10 positioned to potentially contact the viral genome are indicated with asterisks.

FIG. 33A depicts the anellovirus particle structure as shown as a surface rendering. The particle is shown in gray with 5 spikes forming a crown structure, numbered for clarity and colored as in FIG. 31A-31D. The spike domains extend from the core on the 5-fold axis.

FIG. 33B depicts the exterior of the crown structure as shown from the side. Five spike domains are colored as in FIG. 33A. The hydrophobic and hydrophilic conserved residues are colored in light blue and magenta, respectively.

FIG. 33C depicts the same spike domain from FIG. 33B rotated to view residues on the interior of the crown structure.

FIG. 33D depicts the spike domain of Ring 10 (colored as in FIG. 31A-31D) aligned with ORF1 sequences representative of different anellovirus genera (indicated in parentheses). Magenta and black asterisks indicate the surface-exposed residues of the P1 domain and P2 domain, respectively. Below the alignment are >30% consensus residues, or O or γ indicating the residues are >70% hydrophobic or >60% hydrophilic, respectively.

FIG. 34A is a schematic representation of full-length Ring 10 (top), which shows highly heterogeneous particles by negative-stained electron microscopy (bottom). Scale bar=100 nm.

FIG. 34B is a schematic representation of Ring 10 delARM (arginine-rich motif; top), which demonstrates a structural homology virus-like particle (VLP) as shown by negative-stained electron microscopy (bottom). Scale bar=100 nm.

FIG. 34C is a schematic representation of Ring 10 delARM delCTD (top), wherein further truncation of the structural C-terminal domain (Δ552-672) preserves a structured VLP as shown by negative-stained electron microscopy (bottom). Scale bar=100 nm.

FIG. 35 depicts a data processing procedure of the Ring 10 delARM cryogenic electron microscopy (cryo-EM) reconstruction. In short, crYOLO picked 58,391 particles from 11,083 micrographs. Several rounds of 2D classification resulted in 11,185 particles. After Relion de novo initial model reconstruction, Relion 3D refinement was implemented to obtain the orientation parameters. All particles with parameters were fed in a 3D classification. The class with the most abundant particle population resulted in 3.98 Å resolution.

FIG. 36A is a representative negative-stained micrograph of Ring 10 delARM. The micrograph was imaged at NanoImaging Service.

FIG. 36B is a representative cryo-EM micrograph of Ring 10 delARM. The micrograph was imaged at NanoImaging Service.

FIGS. 37A and 37B depict circular dichroism (CD) results of the TTMV-Ring 10 C-terminal peptide (CSEEEEESNLFERLLRQRTKQLQLKRRIIQTLKDLQKLE). FIG. 37A is a table showing averages of secondary structure fractions estimated by different packages of CDPro. α-helix dominates the secondary structure assignment from the CD spectrum. FIG. 37B shows an experimental spectrum of the C-terminal peptide (shown in red) overlaid with the calculated and averaged reference set spectra (shown in blue) from three different packages (SELCON3, CDSSTR, and CONTINLL).

FIG. 38 depicts sequence alignment of 15 known anelloviruses within different genera indicated in parentheses. The conserved amino acids are shown in the first blue row underneath the sequence. The top blue row underneath the sequence alignment indicates the homology sequence if the conservation is larger than 30%. The bottom blue row shows either hydrophobic (0, within 70% similarity) or 60% positive charged (γ, within 60% similarity) of amino acids, respectively. The alignment was done by Geneious-implemented Clustal Omega.

FIG. 39A-39D depict the spikes of Ring 10 or alpha-fold predicted JA20 and MN779270.1.

FIG. 39A depicts the sphere representation of Ring 10 spike (identical to FIG. 33C). FIG. 39B and 39C are the spike representations for alpha-fold predicted JA20 and MN779270.1, respectively. P1 and P2 domains are demonstrated in blue and green. The light blue and magenta are the conserved hydrophobic and basic residues. FIG. 39D shows the sequence alignment between Ring 10, JA20, and MN779210.1.

FIGS. 40A-40B are a series of diagrams showing a Coomassie stain (FIG. A1) and a Western blot (FIG. A2) for Ring2 virus-like particles (VLPs).

FIG. 41 is an electron micrograph showing Ring2 VLPs obtained after Capto400 purification.

FIGS. 42A-42B are a series of diagrams showing a Coomassie stain (FIG. A4) and a Western blot (FIG. A5) for Ring19 VLPs.

FIG. 43 is an electron micrograph showing Ring19 VLPs obtained after Capto400 purification.

FIG. 44 is a diagram showing an exemplary workflow for conjugating NHS ester moieties to surface lysines of anelloVLPs using click chemistry.

FIGS. 45A-45B are a series of diagrams showing a Coomassie stain (FIG. B2) and a Western blot (FIG. B3) for Ring2 VLPs conjugated with NHS Ester 647.

FIGS. 46A-46B are a series of diagrams showing Western blots for Ring2 anelloVLPs conjugated with NHS Ester biotin, labeled using a streptavidin CV 800 antibody (FIG. B4) or a Ring19 HVR3 primary antibody and a goat anti-rabbit secondary antibody (FIG. B5).

FIG. 47 is a diagram showing an exemplary two-step process for conjugating a surface effector moiety to the surface of an anelloVLP.

FIG. 48 is a diagram showing Coomassie staining, Western blot, and UV labeling for SARS-CoV-2 receptor binding domains (RBD) attached to DBCO, CalFluor 488, Azide, or Alexa488, as shown.

FIG. 49 is a diagram showing Coomassie staining and Western blot for pRTx-2652 (a Ring 2 ORF1 variant) produced using SE-FPLC.

FIG. 50 is a diagram showing Coomassie staining and Western blot showing conjugation of RBD attached to an azide moiety to pRTx-2652 ORF1 polypeptide attached to DBCO, to produce RBD-pRTx-2652 conjugate anelloVLPs.

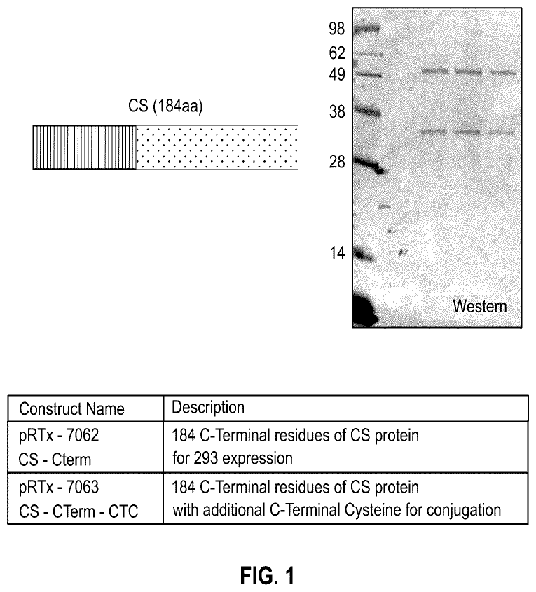

### DETAILED DESCRIPTION OF CERTAIN EMBODIMENTS

### Definitions

The present invention will be described with respect to particular embodiments and with reference to certain figures, but the invention is not limited thereto but only by the claims. Terms as set forth hereinafter are generally to be understood in their common sense unless indicated otherwise.

Where the term “comprising” is used in the present description and claims, it does not exclude other elements. For the purposes of the present invention, the term “consisting of” is considered to be a preferred embodiment of the term “comprising of”. If hereinafter a group is defined to comprise at least a certain number of embodiments, this is to be understood to preferably also disclose a group which consists only of these embodiments.

Where an indefinite or definite article is used when referring to a singular noun, e.g. “a”, “an” or “the”, this includes a plural of that noun unless something else is specifically stated.

The wording “compound, composition, product, etc. for treating, modulating, etc.” is to be understood to refer a compound, composition, product, etc. per se which is suitable for the indicated purposes of treating, modulating, etc. The wording “compound, composition, product, etc. for treating, modulating, etc.” additionally discloses that, as an embodiment, such compound, composition, product, etc. is for use in treating, modulating, etc.

The wording “compound, composition, product, etc. for use in . . . ”, “use of a compound, composition, product, etc in the manufacture of a medicament, pharmaceutical composition, veterinary composition, diagnostic composition, etc. for . . . ”, or “compound, composition, product, etc. for use as a medicament . . . ” indicates that such compounds, compositions, products, etc. are to be used in therapeutic methods which may be practiced on the human or animal body. They are considered as an equivalent disclosure of embodiments and claims pertaining to methods of treatment, etc. If an embodiment or a claim thus refers to “a compound for use in treating a human or animal being suspected to suffer from a disease”, this is considered to be also a disclosure of a “use of a compound in the manufacture of a medicament for treating a human or animal being suspected to suffer from a disease” or a “method of treatment by administering a compound to a human or animal being suspected to suffer from a disease”.

The wording “compound, composition, product, etc. for treating, modulating, etc.” is to be understood to refer a compound, composition, product, etc. per se which is suitable for the indicated purposes of treating, modulating, etc.

If hereinafter examples of a term, value, number, etc. are provided in parentheses, this is to be understood as an indication that the examples mentioned in the parentheses can constitute an embodiment. For example, if it is stated that “in embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1-encoding nucleotide sequence of Table 1 (e.g., nucleotides 571-2613 of the nucleic acid sequence of Table 1)”, then some embodiments relate to nucleic acid molecules comprising a nucleic acid sequence having at least about 70%, 75%, 80%, 85%, 90%, 95%, 96%, 97%, 98%, 99%, or 100% sequence identity to nucleotides 571-2613 of the nucleic acid sequence of Table 1.

As used herein, the term “anellovector” refers to a vehicle comprising a genetic element, e.g., an episome, e.g., circular DNA, enclosed in a proteinaceous exterior. A “synthetic anellovector,” as used herein, generally refers to an anellovector that is not naturally occurring, e.g., has a sequence that is different relative to a wild-type virus (e.g., a wild-type Anellovirus as described herein). In some embodiments, the proteinaceous exterior comprises an ORF1 molecule (e.g., an Anellovirus ORF1 protein), e.g., as described herein. In some embodiments, the proteinaceous exterior comprises a plurality of ORF1 molecules (e.g., an Anellovirus ORF1 protein), e.g., at least about 40, 45, 50, 55, 60, 65, or 70 ORF1 molecules. In some embodiments, the synthetic anellovector is engineered or recombinant, e.g., comprises a genetic element that comprises a difference or modification relative to a wild-type viral genome (e.g., a wild-type Anellovirus genome as described herein). In some embodiments, enclosed within a proteinaceous exterior encompasses 100% coverage by a proteinaceous exterior, as well as less than 100% coverage, e.g., 95%, 90%, 85%, 80%, 70%, 60%, 50% or less. For example, gaps or discontinuities (e.g., that render the proteinaceous exterior permeable to water, ions, peptides, or small molecules) may be present in the proteinaceous exterior, so long as the genetic element is retained in the proteinaceous exterior, e.g., prior to entry into a host cell. In some embodiments, the anellovector is purified, e.g., it is separated from its original source and/or substantially free (>50%, >60%, >70%, >80%, >90%) of other components.

An anellovector may, in some embodiments, comprise a nucleic acid vector that comprises sufficient nucleic acid sequence derived from or highly similar to (e.g., at least 85%, 90%, 95%, 96%, 97%, 98%, 99%, or 100% identical to) an Anellovirus genome sequence or a contiguous portion thereof to allow packaging into a proteinaceous exterior (e.g., a capsid), and further comprises a heterologous sequence. In some embodiments, the anellovector is a viral vector or a naked nucleic acid. In some embodiments, the anellovector comprises at least about 50, 60, 70, 71, 72, 73, 74, 75, 80, 90, 100, 150, 200, 300, 400, 500, 600, 700, 800, 900, 1000, 1100, 1200, 1300, 1400, 1500, 1600, 1700, 1800, 1900, 2000, 2500, 3000, or 3500 consecutive nucleotides of a native Anellovirus sequence or a sequence highly similar (e.g., at least 85%, 90%, 95%, 96%, 97%, 98%, 99%, or 100% identical) thereto. In some embodiments, the anellovector further comprises one or more of an Anellovirus ORF1, ORF2, or ORF3.

In some embodiments, the heterologous sequence comprises a multiple cloning site, comprises a heterologous promoter, comprises a coding region for a therapeutic protein, or encodes a therapeutic nucleic acid. In some embodiments, the capsid is a wild-type Anellovirus capsid. In embodiments, an anellovector comprises a genetic element described herein, e.g., comprises a genetic element comprising a promoter, a sequence encoding a therapeutic effector, and a capsid binding sequence.

As used herein, the term “anelloVLP” refers to a vehicle (e.g., a virus-like particle) comprising a proteinaceous exterior and an effector (e.g., an exogenous effector). In some instances, an anelloVLP does not comprise a substantial amount of a nucleic acid. In some embodiments, the proteinaceous exterior comprises an ORF1 molecule (e.g., an Anellovirus ORF1 protein), e.g., as described herein. In some embodiments, the proteinaceous exterior comprises a plurality of ORF1 molecules (e.g., an Anellovirus ORF1 protein), e.g., at least about 40, 45, 50, 55, 60, 65, or 70 ORF1 molecules. In some embodiments, the effector is enclosed in the proteinaceous exterior. In some embodiments, the effector is on the surface of the proteinaceous exterior (e.g., comprised in a surface moiety as described herein). In some embodiments, the anelloVLP does not comprise a polynucleotide of greater than 50, 100, 150, 200, 300, 400, 500, 600, 700, 800, 900, 1000, 1100, 1200, 1300, 1400, 1500, 1600, 1700, 1800, 1900, or 2000 nucleotides in length. In some embodiments, the anelloVLP does not comprise a polynucleotide comprising an Anellovirus 5′ UTR or Anellovirus origin of replication. In some embodiments, the anelloVLP does not comprise a polynucleotide comprising any contiguous nucleic acid sequences of at least 50, 100, 150, 200, 300, 400, 500, 600, 700, 800, 900, or 1000 nucleotides in length having least 75%, 80%, 85%, 90%, 95%, 96%, 97%, 98%, 99%, or 100% sequence identity to contiguous sequence in a wild-type Anellovirus genome (e.g., as described herein).

As used herein, the term “antibody molecule” refers to a protein, e.g., an immunoglobulin chain or fragment thereof, comprising at least one immunoglobulin variable domain sequence. The term “antibody molecule” encompasses full-length antibodies and antibody fragments (e.g., scFvs). In some embodiments, an antibody molecule is a multispecific antibody molecule, e.g., the antibody molecule comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In embodiments, the multispecific antibody molecule is a bispecific antibody molecule. A bispecific antibody molecule is generally characterized by a first immunoglobulin variable domain sequence which has binding specificity for a first epitope and a second immunoglobulin variable domain sequence that has binding specificity for a second epitope.

The term “disassembly,” as used herein with respect to a particle, such as a virus-like particle (VLP), or a proteinaceous exterior, refers to disassociating one or more components of the particle (e.g., a capsid protein, e.g., an ORF1 molecule as described herein) from the remainder of the particle. In some instances, disassembly of a particle (e.g., a VLP) comprises separating enough of the ORF1 molecules from each other that they no longer form a proteinaceous exterior. In some instances, a ORF1 molecules separated from each other via disassembly of a particle form capsomers (e.g., decameric capsomers), e.g., as described herein. In some embodiments, disassembly reduces the particle to individual monomers. In some embodiments, after disassembly, multimers, e.g., decamers, monomers, and/or pentamers remain. In some instances, disassembly comprises denaturation of protein complexes of the particle (e.g., breaking noncovalent bounds between ORF1 molecules in the proteinaceous exterior). In some instances, disassembly is driven by a denaturant as described herein

The term “in vitro assembly,” as used herein with respect to an anellovector or an anelloVLP, refers to the formation of a proteinaceous exterior comprising an ORF1 molecule, wherein the formation does not take place inside of a cell (e.g., takes place in a cell-free system such as a cell-free suspension, a lysate, or a supernatant). In some instances, in vitro assembly of an anellovector comprises enclosure, outside of a cell, of a genetic element (e.g., as described herein) within the proteinaceous exterior. In some instances, in vitro assembly of an anelloVLP comprises association, outside of a cell, of an effector (e.g., an exogenous effector, e.g., as described herein) with the proteinaceous exterior (e.g., enclosed within the proteinaceous exterior). In vitro assembly of a proteinaceous exterior may occur, in some instances, under conditions suitable for multimerization of a plurality of ORF1 molecules (e.g., nondenaturing conditions), e.g., to form a multimer of more than 10, 11, 12, 13, 14, 15, 16, 17, 18, 19, or 20 ORF1 molecules. In some instances, in vitro assembly results in the formation of a proteinaceous exterior comprising at least about 20, 30, 40, 50, or 60 ORF1 molecules, or about 20-30, 30-40, 40-50, 50-60, or 60-70 ORF1 molecules). In some instances, the proteinaceous exterior is formed from ORF1 molecules that were produced in a cell and then purified therefrom. In some instances, the in vitro assembly takes place in a solution free of cells or constituents thereof. In other instances, the in vitro assembly takes place in a solution comprising cell debris (e.g., from lysed cells). In some instances, the in vitro assembly takes place in a solution substantially free of cellular nucleic acid molecules (e.g., genomic DNA, mitochondrial DNA, mRNA, and/or noncoding RNA from a cell). As used herein, a nucleic acid “encoding” refers to a nucleic acid sequence encoding an amino acid sequence or a functional polynucleotide (e.g., a non-coding RNA, e.g., an siRNA or miRNA).

An “exogenous” agent (e.g., an effector, a nucleic acid (e.g., RNA), a gene, payload, protein) as used herein refers to an agent that is either not comprised by, or not encoded by, a corresponding wild-type virus, e.g., an Anellovirus as described herein. In some embodiments, the exogenous agent does not naturally exist, such as a protein or nucleic acid that has a sequence that is altered (e.g., by insertion, deletion, or substitution) relative to a naturally occurring protein or nucleic acid. In some embodiments, the exogenous agent does not naturally exist in the host cell. In some embodiments, the exogenous agent exists naturally in the host cell but is exogenous to the virus. In some embodiments, the exogenous agent exists naturally in the host cell, but is not present at a desired level or at a desired time.

A “heterologous” agent or element (e.g., an effector, a nucleic acid sequence, an amino acid sequence), as used herein with respect to another agent or element (e.g., an effector, a nucleic acid sequence, an amino acid sequence), refers to agents or elements that are not naturally found together, e.g., in a wild-type virus, e.g., an Anellovirus. In some embodiments, a heterologous nucleic acid sequence may be present in the same nucleic acid as a naturally occurring nucleic acid sequence (e.g., a sequence that is naturally occurring in the Anellovirus). In some embodiments, a heterologous agent or element is exogenous relative to an Anellovirus from which other (e.g., the remainder of) elements of the anellovector are based.

As used herein, the term “genetic element” refers to a nucleic acid sequence, generally in an anellovector. It is understood that the genetic element can be produced as naked DNA and optionally further assembled into a proteinaceous exterior. It is also understood that an anellovector can insert its genetic element into a cell, resulting in the genetic element being present in the cell and the proteinaceous exterior not necessarily entering the cell.

As used herein, the term “ORF1 molecule” refers to a polypeptide having an activity and/or a structural feature of an Anellovirus ORF1 protein (e.g., an Anellovirus ORF1 protein as described herein, e.g., as listed in any one of Tables A1-A25), or a functional fragment thereof. An ORF1 molecule may, in some instances, comprise one or more of (e.g., 1, 2, 3 or 4 of): a first region comprising at least 60% basic residues (e.g., at least 60% arginine residues), a second region compising at least about six beta strands (e.g., at least 4, 5, 6, 7, 8, 9, 10, 11, or 12 beta strands), a third region comprising a structure or an activity of an Anellovirus N22 domain (e.g., as described herein, e.g., an N22 domain from an Anellovirus ORF1 protein as described herein), and/or a fourth region comprising a structure or an activity of an Anellovirus C-terminal domain (CTD) (e.g., as described herein, e.g., a CTD from an Anellovirus ORF1 protein as described herein). In some instances, the ORF1 molecule comprises, in N-terminal to C-terminal order, the first, second, third, and fourth regions. In some instances, an anellovector comprises an ORF1 molecule comprising, in N-terminal to C-terminal order, the first, second, third, and fourth regions. An ORF1 molecule may, in some instances, comprise a polypeptide encoded by an Anellovirus ORF1 nucleic acid (e.g., as listed in any one of Tables N1-N25). An ORF1 molecule may, in some instances, further comprise a heterologous sequence, e.g., a hypervariable region (HVR), e.g., an HVR from an Anellovirus ORF1 protein, e.g., as described herein. An “Anellovirus ORF1 protein,” as used herein, refers to an ORF1 protein encoded by an Anellovirus genome (e.g., a wild-type Anellovirus genome, e.g., as described herein), e.g., an ORF1 protein having the amino acid sequence as listed in any one of Tables A1-A25, or as encoded by the ORF1 gene as listed in any one of Tables N1-N25.

The term “ORF1 domain,” as used herein with respect to an ORF1 molecule, refers to the portion of the ORF1 molecule having the structure or function of an Anellovirus ORF1 protein. The ORF1 domain is generally capable of forming a multimer with other copies of the ORF1 domain (e.g., in other ORF1 molecules), or with other ORF1 molecules, e.g., to form a proteinaceous exterior (e.g., of an anellovector or anelloVLP as described herein). In some instances, the ORF1 molecule may comprise one or more additional domains other than the ORF1 domain (for example, a domain comprising or attached to a surface effector, e.g., as described herein). In some instances, the amino acid sequence of an ORF1 domain comprises an insertion (e.g., an insertion encoding a surface moiety or a domain capable of binding to a surface moiety), e.g., between the N-terminal end and C-terminal end of the ORF1 domain.

In certain instances, the insertion does not substantially disrupt the structure and/or function of the ORF1 domain, e.g., such that the ORF1 domain remains capable of forming a multimer with other ORF1 domains or ORF1 molecules. The position within the ORF1 domain sequence into which the insertion is made is referred to herein as the “insertion point.” An insertion can be made into an ORF1 domain by any genetic or polypeptide engineering method known in the art. In some embodiments, an ORF1 molecule consists of an ORF1 domain. In other embodiments, an ORF1 molecule comprises an ORF1 domain and a heterologous domain (e.g., a surface moiety as described herein). In some embodiments, an ORF1 domain is connected to a surface moiety by a polypeptide linker region.

As used herein, the term “ORF2 molecule” refers to a polypeptide having an activity and/or a structural feature of an Anellovirus ORF2 protein (e.g., an Anellovirus ORF2 protein as described herein, e.g., as listed in any one of Tables A1-A25), or a functional fragment thereof. An “Anellovirus ORF2 protein,” as used herein, refers to an ORF2 protein encoded by an Anellovirus genome (e.g., a wild-type Anellovirus genome, e.g., as described herein), e.g., an ORF2 protein having the amino acid sequence as listed in any one of Tables A1-A25, or as encoded by the ORF2 gene as listed in any one of Tables N1-N25.

As used herein, the term “particle” refers to a vehicle having a diameter of less than 100 nm (e.g., about 20-25, 25-30, 30-35, or 35-40 nm) comprising a proteinaceous exterior. In some instances, the particle comprises a plurality of ORF1 molecules. The proteinaceous exterior of the particle generally forms an enclosure capable of limiting or preventing movement of certain molecules between the inside and outside of the proteinaceous exterior. In some embodiments, gaps or discontinuities (e.g., that render the proteinaceous exterior permeable to water, ions, peptides, or small molecules) may be present in the proteinaceous exterior. In certain embodiments, the gaps or discontinuities are of a sufficiently small size (e.g., diameter) that the proteinaceous exterior limits or prevents one or more large macromolecules (e.g., peptides, polypeptides, polynucleotides, lipids, or polysaccharides) from passing through the proteinaceous exterior.

As used herein, the term “proteinaceous exterior” refers to an exterior component that is predominantly (e.g., >50%, >60%, >70%, >80%, >90%) protein.

As used herein, the term “regulatory nucleic acid” refers to a nucleic acid sequence that modifies expression, e.g., transcription and/or translation, of a DNA sequence that encodes an expression product.

In embodiments, the expression product comprises RNA or protein.

As used herein, the term “regulatory sequence” refers to a nucleic acid sequence that modifies transcription of a target gene product. In some embodiments, the regulatory sequence is a promoter or an enhancer.

As used herein, the term “replication protein” refers to a protein, e.g., a viral protein, that is utilized during infection, viral genome replication/expression, viral protein synthesis, and/or assembly of the viral components.

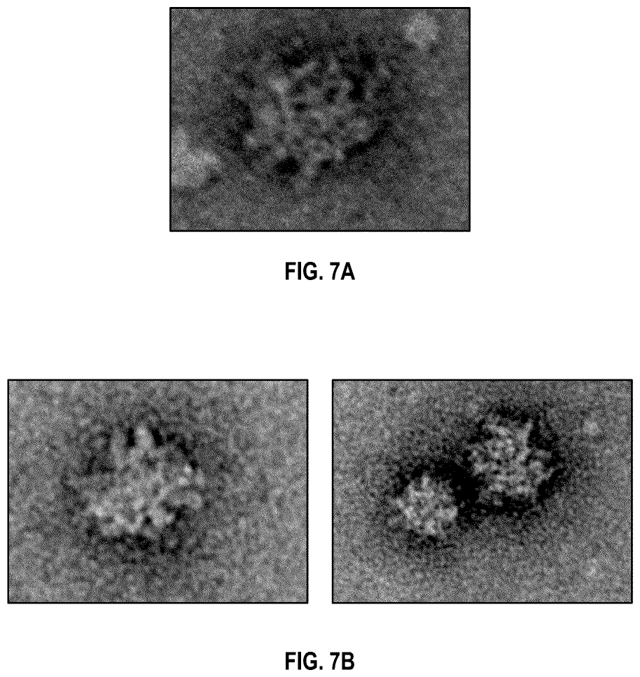

When viewed by electron microscopy, anellovector or anelloVLP particles typically adopt one of two conformations: a symmetrical morphology (e.g., as exemplified in FIG. 7A) and an asymmetrical, or less symmetrical, morphology (e.g., as exemplified in FIG. 7B). Accordingly, the term “symmetrical morphology,” as used herein with respect to anellovector or anelloVLP particle morphology, refers to a particle having a shape that is predominantly symmetrical. The particle having symmetrical morphology may, in some instances, be approximately round. The particle having symmetrical morphology may, in some instances, not be perfectly circular or spherical (e.g., may be ovoid). In some instances, the particle having symmetrical morphology may include one or more deviations from a circular or spherical shape (e.g., one or more protrusions or indentations from its surface).

As used herein, a “substantially non-pathogenic” organism, particle, or component, refers to an organism, particle (e.g., a virus or an anellovector, e.g., as described herein), or component thereof that does not cause or induce a detectable disease or pathogenic condition, e.g., in a host organism, e.g., a mammal, e.g., a human. In some embodiments, administration of an anellovector to a subject can result in minor reactions or side effects that are acceptable as part of standard of care.

As used herein, the term “non-pathogenic” refers to an organism or component thereof that does not cause or induce a detectable disease or pathogenic condition, e.g., in a host organism, e.g., a mammal, e.g., a human.

As used herein, a “substantially non-integrating” genetic element refers to a genetic element, e.g., a genetic element in a virus or anellovector, e.g., as described herein, wherein less than about 0.01%, 0.05%, 0.1%, 0.5%, or 1% of the genetic element that enter into a host cell (e.g., a eukaryotic cell) or organism (e.g., a mammal, e.g., a human) integrate into the genome. In some embodiments the genetic element does not detectably integrate into the genome of, e.g., a host cell. In some embodiments, integration of the genetic element into the genome can be detected using techniques as described herein, e.g., nucleic acid sequencing, PCR detection and/or nucleic acid hybridization.

As used herein, a “substantially non-immunogenic” organism, particle, or component, refers to an organism, particle (e.g., a virus or anellovector, e.g., as described herein), or component thereof, that does not cause or induce an undesired or untargeted immune response, e.g., in a host tissue or organism (e.g., a mammal, e.g., a human). In some embodiments, the substantially non-immunogenic organism, particle, or component does not produce a detectable immune response. In some embodiments, the substantially non-immunogenic anellovector does not produce a detectable immune response against a protein comprising an amino acid sequence or encoded by a nucleic acid sequence shown in any one of Tables N1-N25. In some embodiments, an immune response (e.g., an undesired or untargeted immune response) is detected by assaying antibody presence or level (e.g., presence or level of an anti-anellovector antibody, e.g., presence or level of an antibody against an anellovector as described herein) in a subject, e.g., according to the anti-TTV antibody detection method described in Tsuda et al. (1999; J. Virol. Methods 77: 199-206; incorporated herein by reference) and/or the method for determining anti-TTV IgG levels described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

A “subsequence” as used herein refers to a nucleic acid sequence or an amino acid sequence that is comprised in a larger nucleic acid sequence or amino acid sequence, respectively. In some instances, a subsequence may comprise a domain or functional fragment of the larger sequence. In some instances, the subsequence may comprise a fragment of the larger sequence capable of forming secondary and/or tertiary structures when isolated from the larger sequence similar to the secondary and/or tertiary structures formed by the subsequence when present with the remainder of the larger sequence. In some instances, a subsequence can be replaced by another sequence (e.g., a subseqence comprising an exogenous sequence or a sequence heterologous to the remainder of the larger sequence, e.g., a corresponding subsequence from a different Anellovirus).

As used herein, the term “surface moiety” refers to a moiety for which at least a portion is exposed on the exterior surface of a particle (e.g., exposed to the solution surrounding the particle). The surface moiety is generally attached, directly or indirectly, to a component of the proteinaceous exterior of the particle (e.g., an ORF1 molecule). In some instances, the surface moiety is covalently attached to the component of the proteinaceous exterior of the particle (e.g., the ORF1 molecule). In some instances, the surface moiety is noncovalently attached to the component of the proteinaceous exterior of the particle (e.g., the ORF1 molecule). In some instances, the surface moiety is bound to a binding moiety that is in turn attached (e.g., covalently or noncovalently) to the component of the proteinaceous exterior of the particle (e.g., the ORF1 molecule). In some instances, the surface moiety is comprised in an ORF1 molecule (e.g., is a heterologous domain of an ORF1 molecule). In some instances, a surface moiety is exogenous relative to an Anellovirus (e.g., the Anellovirus from which the ORF1 molecule was derived and/or an Anellovirus for which the ORF1 protein has at least 50%, 60%, 70%, 75%, 80%, 85%, 90%, 95%, 96%, 97%, 98%, 99%, or 100% sequence identity to the ORF1 molecule). In some instances, a surface moiety is exogenous relative a target cell (e.g., a mammalian cell, e.g., a human cell) to be infected by the particle.

As used herein, “treatment”, “treating” and cognates thereof refer to the medical management of a subject with the intent to improve, ameliorate, stabilize, prevent or cure a disease, pathological condition, or disorder. This term includes active treatment (treatment directed to improve the disease, pathological condition, or disorder), causal treatment (treatment directed to the cause of the associated disease, pathological condition, or disorder), palliative treatment (treatment designed for the relief of symptoms), preventative treatment (treatment directed to preventing, minimizing or partially or completely inhibiting the development of the associated disease, pathological condition, or disorder); and supportive treatment (treatment employed to supplement another therapy).

As used herein, the term “virome” refers to viruses in a particular environment, e.g., a part of a body, e.g., in an organism, e.g. in a cell, e.g. in a tissue.

This invention relates generally to anellovectors, e.g., synthetic anellovectors, and uses thereof. The present disclosure provides anellovectors, compositions comprising anellovectors, and methods of making or using anellovectors. Anellovectors are generally useful as delivery vehicles, e.g., for delivering a therapeutic agent to a eukaryotic cell. Generally, an anellovector will include a genetic element comprising a nucleic acid sequence (e.g., encoding an effector, e.g., an exogenous effector or an endogenous effector) enclosed within a proteinaceous exterior. An anellovector may include one or more deletions of sequences (e.g., regions or domains as described herein) relative to an Anellovirus sequence (e.g., as described herein). Anellovectors can be used as a substantially non-immunogenic vehicle for delivering the genetic element, or an effector encoded therein (e.g., a polypeptide or nucleic acid effector, e.g., as described herein), into eukaryotic cells, e.g., to treat a disease or disorder in a subject comprising the cells.

TABLE OF CONTENTS

I. Anellovectors and AnelloVLPs
A. Anelloviruses
B. ORF1 molecules
C. ORF2 molecules
D. Genetic elements
E. Protein binding sequences
F. 5′ UTR Regions
G. GC-rich regions
H. Effectors
I. Proteinaceous exterior
J. Surface moieties
i. Click chemistry
(a) Exemplary click chemistries
(b) Mutations of surface lysines
(c) Mutations of surface cysteines
ii. Genetic grafting
iii. X-fold symmetry
II. Compositions and Methods for Making Anellovectors and AnelloVLPs
A. Components and Assembly of Anellovectors and AnelloVLPs
i. ORF1 molecules for assembly of anellovectors and anelloVLPs
ii. ORF2 molecules for assembly of anellovectors and anelloVLPs
iii. Production of protein components
(a) Baculovirus expression systems
(b) Insect cell systems
(c) Mammalian cell systems
B. Genetic Element Constructs
i. Plasmids
ii. Circular nucleic acid constructs
iii. In vitro circularization
iv. Tandem constructs
v. Cis/trans constructs
vi. Expression cassettes
vii. Design and production of a genetic element construct
C. Effectors
D. Host Cells
i. Introduction of genetic elements into host cells
ii. Methods for providing protein(s) in cis or trans
iii. Exemplary cell types
E. Culture Conditions
F. Harvest
G. In vitro assembly methods for anellovectors
H. In vitro assembly for anelloVLPs
I. Enrichment and Purification
III. Vectors
IV. Compositions
V. Host cells
VI. Methods of use
VII. Methods of production
VIII. Administration/Delivery

### I. Anellovectors and AnelloVLPs

In some aspects, the invention described herein comprises compositions and methods of using and making an anellovector, anellovector preparations, anelloVLPs, anelloVLP preparations, and therapeutic compositions.

### Anellovectors

In some embodiments, the anellovector has a sequence, structure, and/or function that is based on an Anellovirus (e.g., an Anellovirus as described herein, e.g., an Anellovirus comprising a nucleic acid or polypeptide comprising a sequence as shown in any one of Tables A1-A25 or N1-N25), or fragments or portions thereof, or other substantially non-pathogenic virus, e.g., a symbiotic virus, commensal virus, native virus. In some embodiments, an Anellovirus-based anellovector comprises at least one element exogenous to that Anellovirus, e.g., an exogenous effector or a nucleic acid sequence encoding an exogenous effector disposed within a genetic element of the anellovector. In some embodiments, an Anellovirus-based anellovector comprises at least one element heterologous to another element from that Anellovirus, e.g., an effector-encoding nucleic acid sequence that is heterologous to another linked nucleic acid sequence, such as a promoter element. In some embodiments, an anellovector comprises a genetic element (e.g., circular DNA, e.g., single stranded DNA), which comprise at least one element that is heterologous relative to the remainder of the genetic element and/or the proteinaceous exterior (e.g., an exogenous element encoding an effector, e.g., as described herein). An anellovector may be a delivery vehicle (e.g., a substantially non-pathogenic delivery vehicle) for a payload into a host, e.g., a human. In some embodiments, the anellovector is capable of replicating in a eukaryotic cell, e.g., a mammalian cell, e.g., a human cell. In some embodiments, the anellovector is substantially non-pathogenic and/or substantially non-integrating in the mammalian (e.g., human) cell. In some embodiments, the anellovector is substantially non-immunogenic in a mammal, e.g., a human. In some embodiments, the anellovector is replication-deficient. In some embodiments, the anellovector is replication-competent.

In some embodiments the anellovector comprises a curon, or a component thereof (e.g., a genetic element, e.g., comprising a sequence encoding an effector, and/or a proteinaceous exterior), e.g., as described in PCT Application No. PCT/US2018/037379, which is incorporated herein by reference in its entirety.

In an aspect, the invention includes an anellovector comprising (i) a genetic element comprising a promoter element, a sequence encoding an effector, (e.g., an endogenous effector or an exogenous effector, e.g., a payload), and a protein binding sequence (e.g., an exterior protein binding sequence, e.g., a packaging signal), wherein the genetic element is a single-stranded DNA, and has one or both of the following properties: is circular and/or integrates into the genome of a eukaryotic cell at a frequency of less than about 0.001%, 0.005%, 0.01%, 0.05%, 0.1%, 0.5%, 1%, 1.5%, or 2% of the genetic element that enters the cell; and (ii) a proteinaceous exterior; wherein the genetic element is enclosed within the proteinaceous exterior; and wherein the anellovector is capable of delivering the genetic element into a eukaryotic cell.

In some embodiments of the anellovector described herein, the genetic element integrates at a frequency of less than about 0.001%, 0.005%, 0.01%, 0.05%, 0.1%, 0.5%, 1%, 1.5%, or 2% of the genetic element that enters a cell. In some embodiments, less than about 0.01%, 0.05%, 0.1%, 0.5%, 1%, 2%, 3%, 4%, or 5% of the genetic elements from a plurality of the anellovectors administered to a subject will integrate into the genome of one or more host cells in the subject. In some embodiments, the genetic elements of a population of anellovectors, e.g., as described herein, integrate into the genome of a host cell at a frequency less than that of a comparable population of AAV viruses, e.g., at about a 50%, 60%, 70%, 75%, 80%, 85%, 90%, 95%, 100%, or more lower frequency than the comparable population of AAV viruses.

In an aspect, the invention includes an anellovector comprising: (i) a genetic element comprising a promoter element and a sequence encoding an effector (e.g., an endogenous effector or an exogenous effector, e.g., a payload), and a protein binding sequence (e.g., an exterior protein binding sequence), wherein the genetic element has at least 75% (e.g., at least 75, 76, 77, 78, 79, 80, 90, 91, 92, 93, 94, 95, 96, 97, 98, 99, or 100%) sequence identity to a wild-type Anellovirus sequence (e.g., a wild-type Torque Teno virus (TTV), Torque Teno mini virus (TTMV), or TTMDV sequence, e.g., a wild-type Anellovirus sequence as listed in any one of Tables N1-N25); and (ii) a proteinaceous exterior; wherein the genetic element is enclosed within the proteinaceous exterior; and wherein the anellovector is capable of delivering the genetic element into a eukaryotic cell.

In one aspect, the invention includes an anellovector comprising:
a) a genetic element comprising (i) a sequence encoding an exterior protein (e.g., a non-pathogenic exterior protein), (ii) an exterior protein binding sequence that binds the genetic element to the non-pathogenic exterior protein, and (iii) a sequence encoding an effector (e.g., an endogenous or exogenous effector); and
b) a proteinaceous exterior that is associated with, e.g., envelops or encloses, the genetic element.

In some embodiments, the anellovector includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the anellovector modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

In some embodiments, the genetic element comprises a promoter element. In some embodiments, the promoter element is selected from an RNA polymerase II-dependent promoter, an RNA polymerase III-dependent promoter, a PGK promoter, a CMV promoter, an EF-1a promoter, an SV40 promoter, a CAGG promoter, or a UBC promoter, TTV viral promoters, Tissue specific, U6 (pollIII), minimal CMV promoter with upstream DNA binding sites for activator proteins (TetR-VP16, Gal4-VP16, dCas9-VP16, etc). In some embodiments, the promoter element comprises a TATA box. In some embodiments, the promoter element is endogenous to a wild-type Anellovirus, e.g., as described herein.

In some embodiments, the genetic element comprises one or more of the following characteristics: single-stranded, circular, negative strand, and/or DNA. In some embodiments, the genetic element comprises an episome. In some embodiments, the portions of the genetic element excluding the effector have a combined size of about 2.5-5 kb (e.g., about 2.8-4 kb, about 2.8-3.2 kb, about 3.6-3.9 kb, or about 2.8-2.9 kb), less than about 5 kb (e.g., less than about 2.9 kb, 3.2 kb, 3.6 kb, 3.9 kb, or 4 kb), or at least 100 nucleotides (e.g., at least 1 kb).

The anellovectors, compositions comprising anellovectors, methods using such anellovectors, etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaceious exteriors, for example a capsid disclosed in Arch Virol (2007) 152: 1961-1975, to produce anellovectors which can then be used to deliver an effector to cells (e.g., animal cells, e.g., human cells or non-human animal cells such as pig or mouse cells). In embodiments, the effector can silence expression of a factor such as an interferon. The examples further describe how anellovectors can be made by inserting effectors into sequences derived, e.g., from an Anellovirus. It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specifc Anellovirus sequences described in the examples may also be replaced by the Anellovirus sequences described hereinafter. These considerations similarly apply to protein binding sequences, regulatory sequences such as promoters, and the like. Independent thereof, the person skilled in the art will in particular consider such embodiments which are closely related to the examples.

In some embodiments, an anellovector, or the genetic element comprised in the anellovector, is introduced into a cell (e.g., a human cell). In some embodiments, the effector (e.g., an RNA, e.g., an miRNA), e.g., encoded by the genetic element of an anellovector, is expressed in a cell (e.g., a human cell), e.g., once the anellovector or the genetic element has been introduced into the cell. In some embodiments, introduction of the anellovector, or genetic element comprised therein, into a cell modulates (e.g., increases or decreases) the level of a target molecule (e.g., a target nucleic acid, e.g., RNA, or a target polypeptide) in the cell, e.g., by altering the expression level of the target molecule by the cell. In some embodiments, introduction of the anellovector, or genetic element comprised therein, decreases level of interferon produced by the cell. In some embodiments, introduction of the anellovector, or genetic element comprised therein, into a cell modulates (e.g., increases or decreases) a function of the cell. In some embodiments, introduction of the anellovector, or genetic element comprised therein, into a cell modulates (e.g., increases or decreases) the viability of the cell. In some embodiments, introduction of the anellovector, or genetic element comprised therein, into a cell decreases viability of a cell (e.g., a cancer cell).

In some embodiments, an anellovector (e.g., a synthetic anellovector) described herein induces an antibody prevalence of less than 70% (e.g., less than about 60%, 50%, 40%, 30%, 20%, or 10% antibody prevalence). In some embodiments, antibody prevalence is determined according to methods known in the art. In some embodiments, antibody prevalence is determined by detecting antibodies against an Anellovirus (e.g., as described herein), or an anellovector based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anellovirus or an anellovector based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

In some embodiments, a replication deficient, replication defective, or replication incompetent genetic element does not encode all of the necessary machinery or components required for replication of the genetic element. In some embodiments, a replication defective genetic element does not encode a replication factor. In some embodiments, a replication defective genetic element does not encode one or more ORFs (e.g., ORF1, ORF1/1, ORF1/2, ORF2, ORF2/2, ORF2/3, and/or ORF2t/3, e.g., as described herein). In some embodiments, the machinery or components not encoded by the genetic element may be provided in trans (e.g., using a helper, e.g., a helper virus or helper plasmid, or encoded in a nucleic acid comprised by the host cell, e.g., integrated into the genome of the host cell), e.g., such that the genetic element can undergo replication in the presence of the machinery or components provided in trans.

In some embodiments, a packaging deficient, packaging defective, or packaging incompetent genetic element cannot be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein). In some embodiments, the packaging defective genetic element cannot be packaged into a proteinaceous exterior even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein). In some embodiments, a packaging deficient genetic element is packaged into a proteinaceous exterior at an efficiency less than 10% (e.g., less than 10%, 9%, 8%, 7%, 6%, 5%, 4%, 3%, 2%, 1%, 0.5%, 0.1%, 0.01%, or 0.001%) compared to a wild-type Anellovirus (e.g., as described herein), even in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

In some embodiments, a packaging competent genetic element can be packaged into a proteinaceous exterior (e.g., wherein the proteinaceous exterior comprises a capsid or a portion thereof, e.g., comprising a polypeptide encoded by an ORF1 nucleic acid, e.g., as described herein). In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein). In some embodiments, the packaging competent genetic element can be packaged into a proteinaceous exterior in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

In some embodiments, a packaging competent genetic element is packaged into a proteinaceous exterior at an efficiency of at least 20% (e.g., at least 20%, 30%, 40%, 50%, 60%, 70%, 80%, 85%, 90%, 95%, 96%, 97%, 98%, 99%, 100%, or higher) compared to a wild-type Anellovirus (e.g., as described herein) in the presence of factors (e.g., ORF1, ORF1/1, ORF1/2, ORF2, ORF2/2, ORF2/3, or ORF2t/3) that would permit packaging of the genetic element of a wild-type Anellovirus (e.g., as described herein).

### AnelloVLPs

In some embodiments, the anelloVLP has a sequence, structure, and/or function that is based on an Anellovirus (e.g., an Anellovirus as described herein, e.g., an Anellovirus comprising a nucleic acid or polypeptide comprising a sequence as shown in any one of Tables A1-A25), or fragments or portions thereof, or other substantially non-pathogenic virus, e.g., a symbiotic virus, commensal virus, native virus. In some embodiments, an Anellovirus-based anelloVLP comprises at least one element exogenous to that Anellovirus, e.g., an exogenous effector or a nucleic acid sequence encoding an exogenous effector. In some embodiments, the anelloVLP comprises a surface moiety comprising the exogenous effector. In some embodiments, an Anellovirus-based anelloVLP comprises at least one element heterologous to another element from that Anellovirus, e.g., an effector-encoding nucleic acid sequence that is heterologous to another linked nucleic acid sequence, such as a promoter element. An anelloVLP may be a delivery vehicle (e.g., a substantially non-pathogenic delivery vehicle) for a payload into a host, e.g., a human. In some embodiments, the anelloVLP is not capable of replicating in a eukaryotic cell, e.g., a mammalian cell, e.g., a human cell. In some embodiments, the anelloVLP is substantially non-pathogenic and/or substantially non-integrating in the mammalian (e.g., human) cell. In some embodiments, the anelloVLP is substantially non-immunogenic in a mammal, e.g., a human.

In an aspect, the invention includes an anelloVLP comprising a proteinaceous exterior and an effector (e.g., an exogenous effector); wherein the anelloVLP is capable of delivering the exogenous effector into a eukaryotic cell. In some embodiments, the exogenous effector is enclosed within the proteinaceous exterior. In some embodiments, the exogenous effector is comprised in a surface moiety on the surface of the anelloVLP (e.g., as described herein). In some embodiments, the proteinaceous exterior comprises one or more ORF1 molecules (e.g., an Anellovirus ORF1 protein, e.g., as described herein, or a polypeptide having at least 50%, 60%, 70%, 75%, 80%, 85%, 90%, 95%, 96%, 97%, 98%, or 99% sequence identity thereto).

In some embodiments, the anelloVLP includes sequences or expression products from (or having >70%, 75%, 80%, 85%, 90%, 95%, 97%, 98%, 99%, 100% homology to) a non-enveloped, circular, single-stranded DNA virus. Animal circular single-stranded DNA viruses generally refer to a subgroup of single strand DNA (ssDNA) viruses, which infect eukaryotic non-plant hosts, and have a circular genome. Thus, animal circular ssDNA viruses are distinguishable from ssDNA viruses that infect prokaryotes (i.e. Microviridae and Inoviridae) and from ssDNA viruses that infect plants (i.e. Geminiviridae and Nanoviridae). They are also distinguishable from linear ssDNA viruses that infect non-plant eukaryotes (i.e. Parvoviridiae).

In some embodiments, the anelloVLP modulates a host cellular function, e.g., transiently or long term. In certain embodiments, the cellular function is stably altered, such as a modulation that persists for at least about 1 hr to about 30 days, or at least about 2 hrs, 6 hrs, 12 hrs, 18 hrs, 24 hrs, 2 days, 3, days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 60 days, or longer or any time therebetween. In certain embodiments, the cellular function is transiently altered, e.g., such as a modulation that persists for no more than about 30 mins to about 7 days, or no more than about 1 hr, 2 hrs, 3 hrs, 4 hrs, 5 hrs, 6 hrs, 7 hrs, 8 hrs, 9 hrs, 10 hrs,

11 hrs, 12 hrs, 13 hrs, 14 hrs, 15 hrs, 16 hrs, 17 hrs, 18 hrs, 19 hrs, 20 hrs, 21 hrs, 22 hrs, 24 hrs, 36 hrs, 48 hrs, 60 hrs, 72 hrs, 4 days, 5 days, 6 days, 7 days, or any time therebetween.

The anelloVLPs, compositions comprising anelloVLPs, methods using such anelloVLPs, etc., as described herein are, in some instances, based in part on the examples which illustrate how different effectors, for example miRNAs (e.g. against IFN or miR-625), shRNA, etc and protein binding sequences, for example DNA sequences that bind to capsid protein such as Q99153, are combined with proteinaceious exteriors, for example a capsid disclosed in Arch Virol (2007) 152: 1961-1975, to produce anelloVLPs which can then be used to deliver an effector to cells (e.g., animal cells, e.g., human cells or non-human animal cells such as pig or mouse cells). In embodiments, the effector can silence expression of a factor such as an interferon. The examples further describe how anelloVLPs can be made by inserting effectors into sequences derived, e.g., from an Anellovirus. It is on the basis of these examples that the description hereinafter contemplates various variations of the specific findings and combinations considered in the examples. For example, the skilled person will understand from the examples that the specific miRNAs are used just as an example of an effector and that other effectors may be, e.g., other regulatory nucleic acids or therapeutic peptides. Similarly, the specific capsids used in the examples may be replaced by substantially non-pathogenic proteins described hereinafter. The specifc Anellovirus sequences described in the examples may also be replaced by the Anellovirus sequences described hereinafter. These considerations similarly apply to protein binding sequences, regulatory sequences such as promoters, and the like. Independent thereof, the person skilled in the art will in particular consider such embodiments which are closely related to the examples.

In some embodiments, an anelloVLP is introduced into a cell (e.g., a human cell). In some embodiments, the exogenous effector is delivered to the cell. In some embodiments, delivery of the exogenous effector to a cell modulates (e.g., increases or decreases) the level of a target molecule (e.g., a target nucleic acid, e.g., RNA, or a target polypeptide) in the cell, e.g., by altering the expression level of the target molecule by the cell. In some embodiments, delivery of the exogenous effector to a cell modulates (e.g., increases or decreases) a function of the cell. In some embodiments, delivery of the exogenous effector to a cell modulates (e.g., increases or decreases) the viability of the cell. In some embodiments, delivery of the exogenous effector to a cell decreases viability of a cell (e.g., a cancer cell).

In some embodiments, an anelloVLP (e.g., a synthetic anelloVLP) described herein induces an antibody prevalence of less than 70% (e.g., less than about 60%, 50%, 40%, 30%, 20%, or 10% antibody prevalence). In some embodiments, antibody prevalence is determined according to methods known in the art. In some embodiments, antibody prevalence is determined by detecting antibodies against an Anellovirus (e.g., as described herein), or an anelloVLP based thereon, in a biological sample, e.g., according to the anti-TTV antibody detection method described in Tsuda et al. (1999; J. Virol. Methods 77: 199-206; incorporated herein by reference) and/or the method for determining anti-TTV IgG seroprevalence described in Kakkola et al. (2008; Virology 382: 182-189; incorporated herein by reference). Antibodies against an Anellovirus or an anelloVLP based thereon can also be detected by methods in the art for detecting anti-viral antibodies, e.g., methods of detecting anti-AAV antibodies, e.g., as described in Calcedo et al. (2013; Front. Immunol. 4(341): 1-7; incorporated herein by reference).

### Anelloviruses

In some embodiments, an anellovector or anelloVLP, e.g., as described herein, comprises sequences or expression products derived from an Anellovirus. In some embodiments, an anellovector or anelloVLP includes one or more sequences or expression products that are exogenous relative to the Anellovirus. In some embodiments, an anellovector or anelloVLP includes one or more sequences or expression products that are endogenous relative to the Anellovirus. In some embodiments, an anellovector or anelloVLP includes one or more sequences or expression products that are heterologous relative to one or more other sequences or expression products in the anellovector. Anelloviruses generally have single-stranded circular DNA genomes with negative polarity. Anelloviruses have not generally been linked to any human disease. However, attempts to link Anellovirus infection with human disease are confounded by the high incidence of asymptomatic Anellovirus viremia in control cohort population(s), the remarkable genomic diversity within the anellovirus viral family, the historical inability to propagate the agent in vitro, and the lack of animal model(s) of Anellovirus disease (Yzebe et al., Panminerva Med. (2002) 44:167-177; Biagini, P., Vet. Microbiol. (2004) 98:95-101).

Anelloviruses are generally transmitted by oronasal or fecal-oral infection, mother-to-infant and/or in utero transmission (Gerner et al., Ped. Infect. Dis. J. (2000) 19:1074-1077). Infected persons can, in some instances, be characterized by a prolonged (months to years) Anellovirus viremia. Humans may be co-infected with more than one genogroup or strain (Saback, et al., Scad. J. Infect. Dis. (2001)

33:121-125). There is a suggestion that these genogroups can recombine within infected humans (Rey et al., Infect. (2003) 31:226-233). The double stranded isoform (replicative) intermediates have been found in several tissues, such as liver, peripheral blood mononuclear cells and bone marrow (Kikuchi et al., J. Med. Virol. (2000) 61:165-170; Okamoto et al., Biochem. Biophys. Res. Commun. (2002) 270:657-662; Rodriguez-lnigo et al., Am. J. Pathol. (2000) 156:1227-1234).

In some embodiments, an anellovector or anelloVLP as described herein comprises one or more polypeptides (e.g., ORF1 molecules) comprising an amino acid sequence having at least about 50%, 60%, 70%, 75%, 80%, 85%, 90%, 95%, 96%, 97%, 98%, 99%, or 100% sequence identity to an Anellovirus sequence, e.g., as described herein, or a fragment thereof. In embodiments, the polypeptide comprises an amino acid sequence encoded by a nucleic acid sequence selected from a sequence as shown in any one of Tables N1-N25, or a sequence having at least 70%, 75%, 80%, 85%, 90%, 95%, 96%, 97%, 98%, 99%, or 100% sequence identity thereto. In embodiments, the polypeptide comprises a sequence as shown in any one of Tables A1-A25, or a sequence having at least 70%, 75%, 80%, 85%, 90%, 95%, 96%, 97%, 98%, 99%, or 100% sequence identity thereto.

In some embodiments, the genetic element comprises a nucleotide sequence encoding an amino acid sequence or a functional fragment thereof or a sequence having at least about 60%, 70% 80%, 85%, 90% 95%, 96%, 97%, 98%, 99%, or 100% sequence identity to any one of the amino acid sequences described herein, e.g., an Anellovirus amino acid sequence.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to an Anellovirus sequence, e.g., as described herein, or a fragment thereof. In embodiments, the anellovector comprises a nucleic acid sequence selected from a sequence as shown in any one of Tables N1-N25, or a sequence having at least 70%, 75%, 80%, 85%, 90%, 95%, 96%, 97%, 98%, 99%, or 100% sequence identity thereto. In embodiments, the anellovector comprises a polypeptide comprising a sequence as shown in any one of Tables A1-A25, or a sequence having at least 70%, 75%, 80%, 85%, 90%, 95%, 96%, 97%, 98%, 99%, or 100% sequence identity thereto.

In some embodiments, an anellovector as described herein comprises one or more nucleic acid molecules (e.g., a genetic element as described herein) comprising a sequence having at least about 70%, 75%, 80%, 85%, 90%, 95%, 96%, 97%, 98%, 99%, or 100% sequence identity to one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, GC-rich region, or any combination thereof, of any of the Anelloviruses described herein (e.g., an Anellovirus sequence as annotated, or as encoded by a sequence listed, in any one of Tables N1-N25. In some embodiments, the nucleic acid molecule comprises a sequence encoding a capsid protein, e.g., an ORF1, ORF1/1, ORF1/2, ORF2, ORF2/2, ORF2/3, ORF2t/3 sequence of any of the Anelloviruses described herein (e.g., an Anellovirus sequence as annotated, or as encoded by a sequence listed, in any one of Tables N1-N25). In some embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 or ORF2 protein (e.g., an ORF1 or ORF2 amino acid sequence as shown in any one of Tables A1-A25, or an ORF1 or ORF2 amino acid sequence encoded by a nucleic acid sequence as shown in any one of Tables N1-N25). In embodiments, the nucleic acid molecule comprises a sequence encoding a capsid protein comprising an amino acid sequence having at least about 70%, 75%, 80%, 85%, 90%, 95%, 96%, 97%, 98%, 99%, or 100% sequence identity to an Anellovirus ORF1 protein (e.g., an ORF1 amino acid sequence as shown in any one of Tables A1-A25, or an ORF1 amino acid sequence encoded by a nucleic acid sequence as shown in any one of Tables N1-N25).

### Nucleic Acid Sequences

In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF1 nucleotide sequence of any one of Tables N1-N25. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF2 nucleotide sequence of any one of Tables N1-N25. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus ORF3 nucleotide sequence of any one of Tables N1-N25. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus GC-rich region nucleotide sequence of any one of Tables N1-N25. In some embodiments, the nucleic acid molecule comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of any one of Tables N1-N25.

### Amino Acid Sequences Encoded by Nucleic Acid Sequences

In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF1 amino acid sequence of any one of Tables A1-A25. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF2 amino acid sequence of any one of Tables A1-A25. In embodiments, the nucleic acid molecule comprises a nucleic acid sequence encoding an amino acid sequence having at least about 70%, 75%, 80%, 85%, 90%, 95%, 96%, 97%, 98%, 99%, or 100% sequence identity to the Anellovirus ORF3 amino acid sequence of any one of Tables A1-A25.

### Proteins Comprising Amino Acid Sequences

In embodiments, the anellovector described herein comprises a protein having an amino acid sequence having at least about 70%, 75%, 80%, 85%, 90%, 95%, 96%, 97%, 98%, 99%, or 100% sequence identity to the Anellovirus ORF1 amino acid sequence of any one of Tables A1-A25. In embodiments, the anellovector described herein comprises a protein having an amino acid sequence having at least about 70%, 75%, 80%, 85%, 90%, 95%, 96%, 97%, 98%, 99%, or 100% sequence identity to the Anellovirus ORF2 amino acid sequence of any one of Tables A1-A25. In embodiments, the anellovector described herein comprises a protein having an amino acid sequence having at least about 70%, 75%, 80%, 85%, 90%, 95%, 96%, 97%, 98%, 99%, or 100% sequence identity to the Anellovirus ORF3 amino acid sequence of any one of Tables A1-A25. In some embodiments, the ORF1 molecule (e.g., comprised in the anellovector) comprises an Anellovirus ORF1 protein of any one of Tables A1-A25 or a splice variant or post-translationally processed (e.g., proteolytically processed) variant thereof. In some embodiments, the ORF2 molecule (e.g., comprised in the anellovector) comprises an Anellovirus ORF2 protein of any one of Tables A1-A25 or a splice variant or post-translationally processed (e.g., proteolytically processed) variant thereof. In some embodiments, the ORF3 molecule (e.g., comprised in the anellovector) comprises an Anellovirus ORF3 protein of any one of Tables A1-A25 or a splice variant or post-translationally processed (e.g., proteolytically processed) variant thereof.

### Polypeptides Comprising Amino Acid Sequences

In some embodiments, the polypeptide described herein comprises an amino acid sequence having at least about 70%, 75%, 80%, 85%, 90%, 95%, 96%, 97%, 98%, 99%, or 100% sequence identity to an Anellovirus ORF1 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anellovirus ORF1 amino acid sequence of any one of Tables A1-A25.

In some embodiments, the polypeptide described herein comprises an amino acid sequence having at least about 70%, 75%, 80%, 85%, 90%, 95%, 96%, 97%, 98%, 99%, or 100% sequence identity to an ORF1 molecule encoded by an Anellovirus ORF1 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF1 molecule encoded by an Anellovirus ORF1 nucleic acid as listed in any one of Tables A1-A25.

In some embodiments, the polypeptide described herein comprises an amino acid sequence having at least about 70%, 75%, 80%, 85%, 90%, 95%, 96%, 97%, 98%, 99%, or 100% sequence identity to an Anellovirus ORF2 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anellovirus ORF2 amino acid sequence of any one of Tables A1-A25.

In some embodiments, the polypeptide described herein comprises an amino acid sequence having at least about 70%, 75%, 80%, 85%, 90%, 95%, 96%, 97%, 98%, 99%, or 100% sequence identity to an ORF2 molecule encoded by an Anellovirus ORF2 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF2 molecule encoded by an Anellovirus ORF2 nucleic acid as listed in any one of Tables A1-A25.

In some embodiments, the polypeptide described herein comprises an amino acid sequence having at least about 70%, 75%, 80%, 85%, 90%, 95%, 96%, 97%, 98%, 99%, or 100% sequence identity to an Anellovirus ORF3 amino acid sequence described herein. In embodiments, the polypeptide described herein comprises an amino acid sequence having at least about 70%, 75%, 80%, 85%, 90%, 95%, 96%, 97%, 98%, 99%, or 100% sequence identity to the Anellovirus ORF3 amino acid sequence of any one of Tables A1-A25.

In some embodiments, the polypeptide described herein comprises an amino acid sequence having at least about 70%, 75%, 80%, 85%, 90%, 95%, 96%, 97%, 98%, 99%, or 100% sequence identity to an ORF3 molecule encoded by an Anellovirus ORF3 nucleic acid described herein. In some embodiments, the polypeptide described herein comprises an amino acid sequence having at least about 70%, 75%, 80%, 85%, 90%, 95%, 96%, 97%, 98%, 99%, or 100% sequence identity to an ORF3 molecule encoded by an Anellovirus ORF3 nucleic acid as listed in any one of Tables A1-A25.

In some embodiments, the polypeptide comprises an amino acid sequence (e.g., an ORF1, ORF1/1, ORF1/2, ORF2, ORF2/2, ORF2/3, or ORF2t/3 sequence) as shown in any one of Tables A1-A25, or a sequence having at least 70%, 75%, 80%, 85%, 90%, 95%, 96%, 97%, 98%, 99%, or 100% sequence identity thereto.

TABLE N1
Novel Anellovirus nucleic acid sequence (Alphatorquevirus)

| | |
|---|---|
| Name | TTV-RTx1 |
| Genus/Clade | Alphatorquevirus, Clade 6 |
| Accession Number | SRR2167793 |

Full Sequence: 3648 bp
1 10 20 30 40 50
| | | | | |
CGTCACTAACCACGTGACTCCCACAGGCCAACCACAGTGTACGTGATTCA
CTTCCTGGGAGTGGTTTACATTATAATATAAGCAACTGCACTTCCGAATG
GCTGAGTTTTCCACGCCCGTCCGCAGCGAGAACACCACGGAGGGGAGTCC
GCGCGTCCCGTGGGCGGGTGCCGAAGGTGAGTTTACACACCGCAGTCAAG
GGGCAATTCGGGCACGGGACTGGCCGGGCTATGGGCAAGGCTCTTAAAAA
GCTATGTTTCTTGGTAGGCCGTACCGAAAGAAAAGGAAACTGCTACTGCT
ACCACTGCATTCTACACCGAAAACTAGCCGGGTTATGAGCTGGTCTAGGC
CTGTACATAATGCCACAGGCATTGAAAGAAACTGGTGGGAGTCCTGTCTT
AGATCCCACGCAAGTTCTTGTGGCTGCGGTAATTTTGTTAATCATATTAA
TGTACTGGCTAATCGGTATGGCTTTGCTGGTTCCACGGAGACGCCGGGTA
ATCCTCGGCCGAGGCCCCCGGTACTGAGCTCCACCACCAGCACTCCTACC
GATCAATCCAGACCAGCTCTACCATGGCATGGGGATACTGGTGGAGAAGG
CGCTTCTGGAGACCCCGCAGGAGATGGAGAACGTGGCGCCGCAGAAGGAG
ACTACGGCCCAGAAGATCTAGACGCACTTTTCGACGCACTCGACGAAGAG
TAAGGAGGCGACGGTGGGGGAGGCGTGCACGCAGGCGGGGATGGCGACGC
AGGACTTATATTAGAGCCAGGCGACGCAGGAGACGAAAAAGACTTGTACT
GACTCAGTGGCATCCCGCAGTTAGAAGAAAATGTAAAATTACAGGCTACA
TGCCTATAGTATACTGTGGACATGGCAGAGCTAGTTTTAACTATGCCTGG
CACTCTGATGACTGTATAAAACAACCACTACCCTTTGGAGGCTCACTATC
TACAGTGTCCTTCAACCTAAAAGTACTATTTGACGAAAACCAAAGAGGAC
TAAACAAATGGAGCTACCCAAATGACCAACTAGACCTCGCCAGATACAAA
GGCTGTAGACTAACATTTTACAGAAAAAAAAACACAGACTACATAGCTCA
ATATGACATATCAGAACCTTATCAACTAGACAAATATAGCTGTGCAAACT
ATCACCCCTCAAAAATGATGTTTGCAAAAAACAAAATTTTAATTCCTAGC
TATGATACAAAACCTAGAGGCAGACAAAGAGTTAGAGTTAGAATAGGGCC
CCCTAAACTATTTACAGACAAGTGGTACAGTCAATCAGACTTATGCAAGG
TAAACCTTGTGTCACTTGCGGTTTCTGCGGCTTCCTTTCTCCACCCATTC
GGCTCACCACAAACTGCCAACTTTTGTGCAACCTTCCAGGTGCTGCAACC
GTTCTACTACCAGGCTATAGGCATTAGTTCTACAAAACACTCAGAAGTTA
TAGACATTTTATATAAGAAAAATACATACTGGCAAAGCAACATTACCTCT
TGGTTTTTAACTAATGTTAAAAACCCAAAAAATATGTCCACAAAAATGTT
TGAGGACATTAATGTTAAATCAAACAAAGACAGTAATTATGACTGGTTTC
CATTTACCCCATACACTACAGAAAACTATTCAAAAATTCAAAATGCAGCT
CAAGAATACTGGAAATATTTAACTAGTGACCACCCACAAGCTACTAATAG
CAATGAAGGCCTAGTACAACCATGGACTAATGCCACTATAAAACAATATG
AATACCACCTCGGTATGTTTAGTCCTATATTTATAGGACCTACCAGAGCT
AAAACTAAATTTAAAACAGCATACTTTGACTGCACTTATAACCCACTACT
AGACAAAGGAATGGGAAACAGAATATGGTATCAATACGCAACCAAAGCTG
ACACACAAATATCAAAAACAGGGTGCTACTGCATGTTAGAAGACATTCCA
ATATATGCAGCATTTTATGGATACGTAGACTTTATAGAAATGGAAATAGG
TAAAGGACAAGACATTAAAGAGAACGGACTTATTTGCTGCATATGTAGAT
ACACAGACCCCCCAATGTACAATGAACAACATCCAGACATGGGATTTGTA
TTTTATAACACTAACTTTGGAAATGGAAAATGGATAGATGGACGGGGCGA
CATACCTACTTACTGGATGCAAAGATGGAGACCTGTTGTATTATTTCAAA
CTGATGTTATTAGAGACTTAGTAGAAACTGGACCTTTTAGTTACAAAGAT
GACCTAGCAAATACCTCACTGACTATGAAATATGAATTCTATTTTACCTG
GGGCGGAAACCAGGCGTACCACCAGACAATCAAAAACCCTTGTAAAGACG
AAGGTACCGGACCCCATAGACAGCCTAGAGACGTACAAGTTACGGACCCG
ACAACCGTGGGACCTGAATATGTGTTCCACGCGTGGGACTGGAGACGGGG
CTTCCTTAGCGAGCGAGCTCTCAGACGCATGTTCGAAAAACCTCTCAACT
ATGATGAGTATTCTAAAAAACCAAAAAGACCTAGAATATTTCCTCCAACA
GAAACAGAGTCCCGAAACCAAGAGCTCGAAGAAAGCTCGCTTTCAGAGGA
AGAAAAGTCGCTACTCTCCACAGAAGAGATCCAGAAAGAGGAGATACAGC
GACAGTTCAAGCGACAGCTCAAGCGACAGCTGCGCCTCGGGCAGCAGCTC
AAACTCCTCCAACAACAACTCCTCAAGACGCAAGCGGGCCTGCACCTAAA
CCCCCTTTCATATTTCCCGCAATAAATAAAGTGTACCTGTTCCCAGACAG
AGCTCCAAAACCTAAACCCACCTCTGGAGACTGGGAAACAGAGTATGCAG
CTTGCAGTGCCTTTGACAGACCCGCTAGAACCAACCTTAGCTCACCCCCT
TACTACCCAGGAGTACCTACTCCCTGGCAAGTAAAATTCAGCCTTAAATT
TCAATAAAGTGCATTTTTACTACAGCTGGGCCGTGGGAGTTTCACTTGTC
GGTGTCTACCTCTTAAGGTCACTAAGCACTCCGAGCGCAGCGAGGAGTGC
GACCCTTAACCCTGGGTCAACGCCTTCGGAGCCGCGCGCTACGCCTTCGG
CTGCGCGCGGCACCTCAGACCCCCGCTCGTGCTGACGCGCTTGCGCGCGT
CAGACCACTTCGGGCTCGCGGGGGTCGGGAACTTTGCTAACAGACTCCGA
GGTGCCATTGGACACAGAGTGGGCGTTCAGCAACGAAAGTGAGTGGGGCC
AGACTTCGCCATAAGGCCTTTATCTTCTTGCCATTTGTCAGTATAAGGGG
TTGCCATAGGCTTCGGCCTCAATTTTAGGCCTTCCGGACTACCAAAATGG
CCGATTTAGTGACGTCACGGCGGCCATTTTAAGTAAGGCGGAAGTAACTC
CACTATTTACAAAATGGCGGCGGAGCACTTCCGGCTTGCCCAAAATGGCG
GCAAAAAACATCCGGGTCAAAGGTCGTTACCACGTCACAAGTCACGTGGG
AGGGTGGTGCTGTAAACCCGGAAGCAATCCTCTCACGTGGCTAGTCACGT
GACTAACACGTCACACCCGCCATTTTGTTTTACAAAATGGCCGACTTCCT
TCCGCTTTTTTAAAAATAACGGCTCAGCGGCGGCGCGCGCGCTACGCG
(SEQ ID NO: 830)
Annotations:

| Putative Domain | Base range |
|---|---|
| TATA Box | 77-81 |
| Initiator Element | 95-110 |
| Transcriptional Start Site | 105 |
| 5′ UTR Conserved Domain | 165-235 |
| ORF2 | 335-703 |
| ORF2/2 | 335-699; 2326-2759 |
| ORF2/3 | 335-699; 2552-2957 |
| ORF2t/3 | 335-465; 2552-2957 |
| ORF1 | 574-2775 |
| ORF1/1 | 574-699; 2326-2775 |
| ORF1/2 | 574-699; 2552-2759 |
| Three open-reading frame region | 2535-2746 |
| Poly(A) Signal | 2953-2958 |
| GC-rich region ** | 3620-3648 |

TABLE A1
Novel Anellovirus amino acid sequences (Alphatorquevirus, Clade 6)
TTV-RTx1 (Alphatorquevirus Clade 6)

| | |
|---|---|
| ORF2 | MSWSRPVHNATGIERNWWESCLRSHASSCGCGNFVNHINVLANRYGFAGS |
| | TETPGNPRPRPPVLSSTTSTPTDQSRPALPWHGDTGGEGASGDPAGDGERGA |
| | AEGDYGPEDLDALFDALDEE (SEQ ID NO: 831) |
| ORF2/2 | MSWSRPVHNATGIERNWWESCLRSHASSCGCGNFVNHINVLANRYGFAGS |
| | TETPGNPRPRPPVLSSTTSTPTDQSRPALPWHGDTGGEGASGDPAGDGERGA |
| | AEGDYGPEDLDALFDALDEEQSKTLVKTKVPDPIDSLETYKLRTRQPWDLN |
| | MCSTRGTGDGASLASELSDACSKNLSTMMSILKNQKDLEYFLQQKQSPETK |
| | SSKKARFQRKKSRYSPQKRSRKRRYSDSSSDSSSDSCASGSSSNSSNNNSSRR |
| | KRACT (SEQ ID NO: 832) |
| ORF2/3 | MSWSRPVHNATGIERNWWESCLRSHASSCGCGNFVNHINVLANRYGFAGS |
| | TETPGNPRPRPPVLSSTTSTPTDQSRPALPWHGDTGGEGASGDPAGDGERGA |
| | AEGDYGPEDLDALFDALDEENRVPKPRARRKLAFRGRKVATLHRRDPERG |
| | DTATVQATAQATAAPRAAAQTPPTTTPQDASGPAPKPPFIFPAINKVYLFPD |
| | RAPKPKPTSGDWETEYAACSAFDRPARTNLSSPPYYPGVPTPWQVKFSLKF |
| | Q (SEQ ID NO: 833) |
| ORF2t/3 | MSWSRPVHNATGIERNWWESCLRSHASSCGCGNFVNHINVLANRNRVPKP |
| | RARRKLAFRGRKVATLHRRDPERGDTATVQATAQATAAPRAAAQTPPTTTP |
| | QDASGPAPKPPFIFPAINKVYLFPDRAPKPKPTSGDWETEYAACSAFDRPART |
| | NLSSPPYYPGVPTPWQVKFSLKFQ (SEQ ID NO: 834) |
| ORF1 | MAWGYWWRRRFWRPRRRWRTWRRRRRLRPRRSRRTFRRTRRRVRRRRW |
| | GRRARRRGWRRRTYIRARRRRRRKRLVLTQWHPAVRRKCKITGYMPIVYC |
| | GHGRASFNYAWHSDDCIKQPLPFGGSLSTVSFNLKVLFDENQRGLNKWSYP |
| | NDQLDLARYKGCRLTFYRKKNTDYIAQYDISEPYQLDKYSCANYHPSKMM |
| | FAKNKILIPSYDTKPRGRQRVRVRIGPPKLFTDKWYSQSDLCKVNLVSLAVS |
| | AASFLHPFGSPQTANFCATFQVLQPFYYQAIGISSTKHSEVIDILYKKNTYWQ |
| | SNITSWFLTNVKNPKNMSTKMFEDINVKSNKDSNYDWFPFTPYTTENYSKIQ |
| | NAAQEYWKYLTSDHPQATNSNEGLVQPWTNATIKQYEYHLGMFSPIFIGPT |
| | RAKTKFKTAYFDCTYNPLLDKGMGNRIWYQYATKADTQISKTGCYCMLED |
| | IPIYAAFYGYVDFIEMEIGKGQDIKENGLICCICRYTDPPMYNEQHPDMGFVF |
| | YNTNFGNGKWIDGRGDIPTYWMQRWRPVVLFQTDVIRDLVETGPFSYKDD |
| | LANTSLTMKYEFYFTWGGNQAYHQTIKNPCKDEGTGPHRQPRDVQVTDPT |
| | TVGPEYVFHAWDWRRGFLSERALRRMFEKPLNYDEYSKKPKRPRIFPPTET |
| | ESRNQELEESSLSEEEKSLLSTEEIQKEEIQRQFKRQLKRQLRLGQQLKLLQQ |
| | QLLKTQAGLHLNPLSYFPQ (SEQ ID NO: 835) |
| ORF1/1 | MAWGYWWRRRFWRPRRRWRTWRRRRRLRPRRSRRTFRRTRRRTIKNPCK |
| | DEGTGPHRQPRDVQVTDPTTVGPEYVFHAWDWRRGFLSERALRRMFEKPL |
| | NYDEYSKKPKRPRIFPPTETESRNQELEESSLSEEEKSLLSTEEIQKEEIQRQFK |
| | RQLKRQLRLGQQLKLLQQQLLKTQAGLHLNPLSYFPQ (SEQ ID NO: 836) |
| ORF1/2 | MAWGYWWRRRFWRPRRRWRTWRRRRRLRPRRSRRTFRRTRRRKQSPETK |
| | SSKKARFQRKKSRYSPQKRSRKRRYSDSSSDSSSDSCASGSSSNSSNNNSSRR |
| | KRACT (SEQ ID NO: 837) |

TABLE N2
Novel Anellovirus nucleic acid sequence (Alphatorquevirus)

| | |
|---|---|
| Name | TTV-RTx2 |
| Genus/Clade | Alphatorquevirus, Clade 6 |
| Accession Number | SRR3479021 |

Full Sequence: 3704 bp
1 10 20 30 40 50
| | | | | |
CCCCGAAGTCCGTCACTAACCACGTGACTCCCACAGGCCAATCAGATGCT
ATGTCGTGCACTTCCTGGGCTGTGTCTACGTCCTCATATAAGTAACTGCA
CTTCCGAATGGCTGAGTTTTCCACGCCCGTCCGCAGCGGCAGCACCACGG
AGGGTGATCCCCGCGTCCCGTGGGCGGGTGCCGGAGGTGAGTTTACACAC
CGCAGTCAAGGGGCAATTCGGGCACGGGACTGGCCGGGCTATGGGCAAGG
CTCTTAAAAAGCTATGTTCTTCGGTAGGTGCTGGAGAAAGAAAAGGAAAG
TGCTTCTGCAAGATCTGTCAACTCCACCGAAAAAACCTGCTATGAGTGTG
TGGCTTCCTCCCATAGACAATGTTACCGAGCGTGAGAGGAGCTGGCTCTC
TAGCATTCTTCAGTCTCACAGAGCTTTTTGTGGGTGCCATGATGCTATCT
ATCATCTTAGCAGTCTGGCTGCTCGCTTTAATATGCAACCAGGGCCGTCG
CCGGGTGGTGATTCTAGGCCGCCGCGACCGCCACTAAGACGCCTGCCCGC
GCTCCCGGGTCCCAGAGACCCCCCTAGCGACACCAACAACCGCAGGTCAT
GGCCTACTGGGGATGGTGGAGACGGAGGCGCTGGCCAAGGCGCAGGTGGA
GGCGCTACCGCTACCGAAGAAGACTACCGCGCCGAAGACCTAGACGAGCT
GTACGCCGCCCTCGAAGGAGACGAGTAAGGAGGCGCCGCGGTAGGGGGTG
GTACAGAGGGCGACGCTACTCCCGCAGACGGTACAGACGTAGATATGTGA
GGCGAAAGAGAAAGACTCTAGTTTGGAGACAGTGGCAGCCTCAAAATATC
AGAAAATGCAGGATCAGGGGCATAATTCCCATCCTGATATGCGGACACGG
GAGGGGGGCCAGAAACTATGCGCTCCACAGCGACGACATAACCCCCCAGA
ACACCCCCTTCGGGGGAGGACTGAGCACCACCTCCTGGAGCCTAAAAGTG
CTATATGACCAGCACACCAGGGGACTCAACAGGTGGTCTGCCAGTAACGA
GAGCCTAGACCTTGCCAGATACAATGGCTGTAGTTTCACTTTCTACAGAG
ACAAAAAGACTGACTTTATAGTGACCTATGACACCTCTGCTCCCTACAAA
CTAGACAAATACAGCTCCCCCAGCTACCACCCAGGGTCCATGATGCTCAT
GACAAAACACAAAATCCTGATCCCCAGTTTTGACACAAAACCCAAAGGTC
CTGCCAAAATTAGAGTCAGAATCAAGCCCCCCAAAATGTTCTTAGATAAA
TGGTACACTCAAGACGACCTCTGTTCCGTTAATCTTGTGTCACTTGCGGT
TAGCGCAGCTTCCTTTACACATCCGTTCTGCCCACCACTAACTGACACTC
CTTGTGTAACGCTGCAGGTGTTGAAAGACTTCTACTACACAACCATAGGC
TACTCCTCTAATGCAGACAAAGTAGAGTCTGTATTCACTAACACTCTCTA
CAAACACTGCTGCTACTATCAGTCCTTTCTCACCACTCAATTTATAGCCA
AAATCACTCGCACACCAGATGGACAACCAGTAGCCACATTCTCTCCTCCT
ACCTCTTTCCCTGGCACAACTGTAACAAAAAGTTCCATAGAATCATTTAA
CCAATGGGTAACTTCCACAGGTACAAGTGGCTGGCTAACAAATGCAAACC
AACACTTTCATTTCTGTAACTATAAACCAGATGCCACAAAGCTAAAATGG
CTCAGACAGTACTACTTTGACTGGGAAACATACAAATTAGCAGATGTAAA
GCCAGACGGCCTTACACCCTCAGTAAACTGGTATGAGTACAGAATAGGCC
TCTTTAGTCCTATTTTCCTGAGCCCCTTCAGATCTAGCAGTCTAGACTTT
CCCAGAGCCTACCAGGATGTGAACTACAACCCCCTGGTAGACAAAGGAGT
GGGCAACATCATATGGTTCCAATACAACACAAAACCAGACACACAGCTGT
CAGTACCCAGCTGCAAGTGTGTCATAGAAGACAAACCCCTATGGGCAGCC
TTCTATGGCTACAGTGACTTTGTACAACAAGAGATAGGAGACTACACAGA
CGCAGAGGCCGTGGGCTTCGTCTGTGTCATCTGTCCATACACCAAACCCC
CTCTAAAAAACCCAGACAACCCCATGCAAGGGTTCATATTCTATGACAGC
CTTTTTGGCAATGGCAAGTGGATAGATGGCACGGGGCACGTCCCCCTTTA
CTGGCAGAGCAGGTGGAGGCCAGAGATGCTCTTCCAAGAAAACACCATGA
GAGACATCACACTATCTGGGCCCTTCAGCTACAAGGACGACTATAAGAAC
TGTGTACTGACTTGCAAATACAAATTTAACTTTCGATTCGGGGGCAATCT
TCTCCACGAACAGACGATCAGAAACCCATGCCCCACGGACGGACATCCCA
GTACCGGTAGACAGCCTAGAGACGTACAAGTGGTTGACCCGATCAAAGTG
GGCCCCCGGTTCGTGTTCCACTCCTGGGACTGGCGCAGAGGCTACCTTAG
CCCAGCAGCTCTCAAAAGAATTGGAGAGCAACCGCTCGATTATGAAGCTT
ATTCGTACCGCCCAAAGAGACCTAGAATCTTTCCTCCCACAGAAGGAGAC
CAGCTCGCCCGAAGTCGAGAAGAAGACTCATTTTCAGAGGAAGAAAGTCC
CCATATCTCGTTCGAAGAGGGGCAGGAACCGAAAGCCCAGGCGGTACAGC
AGCACCTCCTCCGACACCTCAGAAAGCAGCGAGAACTCCGAAAGCGACTC
CGAGCCCTGTTCCAAAGCCTCCAAAAGACGCAGGCGGGTCTCCACGTAAA
TCCATTATTATTCAACCAGCCTGCAATCAGGTTCTGATGTTCCCAGAGAT
GGGGCCTAAGCCAGCTCCCACTGCCCAAGACTGGCAGTGCGAATACGAGA
CATGTAAGCACTGGGATAGACCCCCCAGAAAGTTTCTCACAGACCCCCCT
TTCTATCCCTGGGCCCCTACTACTTACAATGTATCTTTCAAGCTAAACTT
CAAATAAACTAGGCCGTGGGAGTCTCACTTGTCGGTGTCTACCTCTTAAG
GTCACTAAGCACTCCGAGCGTCAGCGAGGAGTGCGACCCTTCCCCCTGGT
GCAACGCCCTCGGCGGCCGCGCGCTACGCCTTCGGCTGCGCGCGGCACCT
CGGACCCCCGCTCGTGCTGACGCGCTCGCGCGCGTCAGACCACTTCGGGC
TCGCGGGGGTCGGGAAATTTGCTAAACAGACTCCGAGTTGCCATTGGACA
CAGGAGCTGTGAATCAGTAACGAAAGTGAGTGGGGCCAGACTTCGCCATA
AGGCCTTTATCTTCTTGCCATTTGTCCGTGAGGAGGGGTCGCCAAGACGC
GGACCCCGTTTTCGGACCTTCCGAACTACCAAAATGGCCGATTCAGTGAC
GTCACGGCAGCCATTTTGTGTAAGCACCGCCCAGGACAGACGTCACAGTT
CAAAGGTCATCCTCGAGCGGAACTTACAGAAAATGGCGGTCAATTGCTTC
CGGGTCAAAGGTCACGCCTACGTCATAAGTCACGTGGTGGAGGCTACTGC
GCATACACGGAAGTAGGCCCCGCCACGTGACCGACCACGTGGGTGCTGCG
TCACGGCCGCCATTTTGTATCACAAAATGGCCGACTTCCTTCCTCTTTTT
CAAA (SEQ ID NO: 838)
Annotations:

| Putative Domain | Base range |
|---|---|
| TATA Box | 87-91 |
| Initiator Element | 105-120 |
| Transcriptional Start Site | 115 |
| 5′ UTR Conserved Domain | 175-245 |
| ORF2 | 342-728 |
| ORF2/2 | 342-724; 2414-2849 |
| ORF2/3 | 342-724; 2643-3057 |
| ORF1 | 599-2887 |
| ORF1/1 | 599-724; 2414-2887 |
| ORF1/2 | 599-724; 2643-2849 |
| Three open-reading frame region | 2626-2846 |
| Poly(A) Signal | 3052-3058 |

TABLE A2
Novel Anellovirus amino acid sequences (Alphatorquevirus, Clade 6)
TTV-RTx2 (Alphatorquevirus Clade 6)

| | |
|---|---|
| ORF2 | MSVWLPPIDNVTERERSWLSSILQSHRAFCGCHDAIYHLSSLAARFNMQPGP |
| | SPGGDSRPPRPPLRRLPALPGPRDPPSDTNNRRSWPTGDGGDGGAGQGAGG |
| | GATATEEDYRAEDLDELYAALEGDE (SEQ ID NO: 839) |
| ORF2/2 | MSVWLPPIDNVTERERSWLSSILQSHRAFCGCHDAIYHLSSLAARFNMQPGP |
| | SPGGDSRPPRPPLRRLPALPGPRDPPSDTNNRRSWPTGDGGDGGAGQGAGG |
| | GATATEEDYRAEDLDELYAALEGDERSETHAPRTDIPVPVDSLETYKWLTR |
| | SKWAPGSCSTPGTGAEATLAQQLSKELESNRSIMKLIRTAQRDLESFLPQKE |
| | TSSPEVEKKTHFQRKKVPISRSKRGRNRKPRRYSSTSSDTSESSENSESDSEPC |
| | SKASKRRRRVST (SEQ ID NO: 840) |
| ORF2/3 | MSVWLPPIDNVTERERSWLSSILQSHRAFCGCHDAIYHLSSLAARFNMQPGP |
| | SPGGDSRPPRPPLRRLPALPGPRDPPSDTNNRRSWPTGDGGDGGAGQGAGG |
| | GATATEEDYRAEDLDELYAALEGDERRPARPKSRRRLIFRGRKSPYLVRRG |
| | AGTESPGGTAAPPPTPQKAARTPKATPSPVPKPPKDAGGSPRKSIIIQPACNQ |
| | VLMFPEMGPKPAPTAQDWQCEYETCKHWDRPPRKFLTDPPFYPWAPTTYN |
| | VSFKLNFK (SEQ ID NO: 841) |
| ORF1 | MAYWGWWRRRRWPRRRWRRYRYRRRLPRRRPRRAVRRPRRRRVRRRRG |
| | RGWYRGRRYSRRRYRRRYVRRKRKTLVWRQWQPQNIRKCRIRGIIPILICGH |
| | GRGARNYALHSDDITPQNTPFGGGLSTTSWSLKVLYDQHTRGLNRWSASNE |
| | SLDLARYNGCSFTFYRDKKTDFIVTYDTSAPYKLDKYSSPSYHPGSMMLMT |
| | KHKILIPSFDTKPKGPAKIRVRIKPPKMFLDKWYTQDDLCSVNLVSLAVSAA |
| | SFTHPFCPPLTDTPCVTLQVLKDFYYTTIGYSSNADKVESVFTNTLYKHCCY |
| | YQSFLTTQFIAKITRTPDGQPVATFSPPTSFPGTTVTKSSIESFNQWVTSTGTS |
| | GWLTNANQHFHFCNYKPDATKLKWLRQYYFDWETYKLADVKPDGLTPSV |
| | NWYEYRIGLFSPIFLSPFRSSSLDFPRAYQDVNYNPLVDKGVGNIIWFQYNTK |
| | PDTQLSVPSCKCVIEDKPLWAAFYGYSDFVQQEIGDYTDAEAVGFVCVICPY |
| | TKPPLKNPDNPMQGFIFYDSLFGNGKWIDGTGHVPLYWQSRWRPEMLFQE |
| | NTMRDITLSGPFSYKDDYKNCVLTCKYKFNFRFGGNLLHEQTIRNPCPTDGH |
| | PSTGRQPRDVQVVDPIKVGPRFVFHSWDWRRGYLSPAALKRIGEQPLDYEA |
| | YSYRPKRPRIFPPTEGDQLARSREEDSFSEEESPHISFEEGQEPKAQAVQQHLL |
| | RHLRKQRELRKRLRALFQSLQKTQAGLHVNPLLFNQPAIRF (SEQ ID NO: |
| | 842) |
| ORF1/1 | MAYWGWWRRRRWPRRRWRRYRYRRRLPRRRPRRAVRRPRRRRTIRNPCP |
| | TDGHPSTGRQPRDVQVVDPIKVGPRFVFHSWDWRRGYLSPAALKRIGEQPL |
| | DYEAYSYRPKRPRIFPPTEGDQLARSREEDSFSEEESPHISFEEGQEPKAQAV |
| | QQHLLRHLRKQRELRKRLRALFQSLQKTQAGLHVNPLLFNQPAIRF (SEQ ID |
| | NO: 843) |
| ORF1/2 | MAYWGWWRRRRWPRRRWRRYRYRRRLPRRRPRRAVRRPRRRRKETSSPE |
| | VEKKTHFQRKKVPISRSKRGRNRKPRRYSSTSSDTSESSENSESDSEPCSKAS |
| | KRRRRVST (SEQ ID NO: 844) |

TABLE N3
Novel Anellovirus nucleic acid sequence (Alphatorquevirus)

| | |
|---|---|
| Name | TTV-RTx3 |
| Genus/Clade | Alphatorquevirus, Clade 4 |
| Accession Number | SRR3479781 |

Full Sequence: 3653 bp
10 20 30 40 50 60
| | | | | |
CCAACCAGAGTCTATGTCGTGCACTTCCTGGGCATGGTCTACGTAATAAT
ATAAAGCGGTGCACTTCCGAATGGCTGAGTTTTCCACGCCCGTCCGCAGC
GAGATCGCGACGGAGGAGCGATCGAGCGTCCCGAGGGCGGGTGCCGGAGG
TGAGTTTACACACCGCAGTCAAGGGGCAATTCGGGCTCGGGACTGGCCGG
GCTATGGGCAAGGCTCTTAAAAAGCCATGTTTCTCGGTAAACTTTACAGG
CAGAAAAGGAAACTGCTACTGCAGCCTGTGCGTGCTCCACAGACGCCATC
TTCCATGAGCTCTACCTGGCGAGTGCCCCGCGGCGATGTCTCCGCCCGCG
AGCTATGTTGGTACCGCTCAGTTCGAGAGAGCCACGATGCTTTTTGTGGC
TGTCGTGATCCTGTTTTTCATCTTTCTCGTCTGGCTGCACGTTCTAACCA
TCAGGGACCTCCGACGCCCCCCACGGACGAGCGCCCGTCGGCGTCTACCC
CAGTGAGGCGCCTGCTGCCGCTGCCCTCCTACCCCGGCGAGGGTCCCCAG
GCTAGATGGCCTGGTGGGGATGGAGAAGGCGCTGGTGGCGCCCGCGGAGG
CGCTGGAGATGGCGGCGCCCGCGCAGGCGAAGAAGAGTACCGGCCCGAAG
ACCTCGACGAGCTGTTCGACGCTATCGAACAAGAACAGTAAGGAGACGGA
GGCGAGGGTGGCGGAGGGGCTACAGGCGCCGTTACAGACTGAGACGCTAC
CGTAGAAGGGGCAGGCGACGCAAAAAAATAGTACTGACTCAGTGGAACCC
CCAGACTGTCAGAAAGTGCTTTATCAGAGGACTGATGCCAGTACTATGGG
CGGGCATGGGCACGGGGGGCCACAACTACGCCGTCCGCTCAGATGACTTT
GTGGTAGACAGAGGCTTCGGGGGCTCCTTCGCCACAGAAACTTTCTCCCT
GAGGGTCCTCTTTGACCAGTACCAGAGAGGATTTAATAGGTGGTCTCACA
CCAACGAAGACCTAGACCTGGCCCGCTACACGGGCTGCAAATGGACATTT
TACAGACACCAAGACACAGACTTTATAGTGTACTTTACAAACAATCCCCC
CATGAAAACCAACCAGCACACAGCCCCTCTCACAACTCCAGGCATGCTCA
TGAGGAGCAAGTATAAAATACTAGTGCCCAGTTTTAAAACAAGACCAAAG
GGCAGAAAAACAGTGTCAGTGAGAGTTAGACCCCCCAAACTGTTTCAGGA
CAAATGGTATACTCAACAGGACCTCTGTCCAGTACCCCTCGTCCAACTGA
ACGTGACCGCAGCGGATTTCACACATCCGTTCGGCTCACCACTAACTGAC
ACGCCTTGCATAAGATTCCAAGTTTTAGGGAACTTATACAACAAGTGCCT
AAATATAGATCTTCCGCAATTTGATGAGGACGGTGAGATACTCACTTCAA
CACCTTATAACAGAGAAAACAAAGAAGATCTTAAAAAGCTTTATAAAACT
CTATTTGTAGATGAACACGCAGGCAATTATTGGCAGACATTCTTAACCAA
CACAATGGTAAAGTCACACATAGATGCAAACCAAGCAAAGACATACGATC
AAGAAAAAACTGCTGCAGAACAAGGTAAAGACCCCTTCCCAACAAACCCA
CCAAAAGACCAATTCACTACCTGGAACAAGAAACTAGTAGACCCTAGAGA
CAGCAACTTTCTCTTTGCCACATATCACCCAAAAAACATTAAAAAAGCTA
TAAAAACCATGAGAGACAACAACTTTGCTCTCACCACAGGCAAAAATGAC
ATATATGGAGACTACACCGCGGCCTACACCAGAAACACCCACATGCTAGA
CTACTACCTAGGCTTTTATAGCCCCATATTTCTTTCCAGCGGTAGGTCCA
ACACAGAGTTCTGGACCGCCTACAGAGACATAGTATATAATCCCCTCTTA
GACAAAGGCACAGGCAACATGATCTGGTTCCAATATCACACAAAAACAGA
CAATATATACAAAAAACCAGAGTGCCACTGGGAGATACTAGACATGCCCC
TGTGGGCCCTCTGCAACGGGTATGTAGAGTACCTAGAGAGCCAAATAAAG
TACGGGGACATCCTAGTAGAGGGCAAAGTCCTCATCAGATGCCCCTACAC
CAAACCCGCACTGGTAGACCCCAATAACAGCCTAGCTGGTTACGTGGTAT
TCAACACCACCTTCGGCCAGGGAAAATGGATAGATGGCAAAGGCTACATC
CCCCTACACGAGAGGAGCAAGTGGTACGTCATGCTCAGATACCAGACCGA
CGTACTCCATGACATAGTGACTTGTGGACCCTGGCAGTACAGAGACGATA
ACAAAAACTCTCAGCTAATAGCCAAGTACAGATTCAAGTTCTACTGGGGA
GGTAACATGGTACATTCTCAGGTCATCAGAAACCCGTGCAAAGACACCCA
AGTATCCGGACCCCGTCGACAGCCTCGCGAAGTACAAGTCGTTGACCCGC
AACTCATTACGCCGCCGTGGGTCCTCCACTCGTTCGACCAGAGACGAGGA
ATGTTTACTGCAGGAGCTATCAAACGTCTGCTCAAGCAACCAATACCTGG
CGAGTATGCTCCTACACCACTCAGGGTCCCGCTCCTCTTTCCCTCCTCAG
AGTTCCAGCGAGAGGGAGAAGATGCAGAAAGCGGCTCAGGTTCACCACCC
AAGAGACCGCGACTCTGGCAGGAAGAGGCCAACCAGACGCAAACGGAGTC
CTCGGAGGGGCCGGCGGAGACGACGAGGGAGCTCCTCGAGCGAAAGCTCA
GAGAGCAGCGAGTCCTCAACCTCCAACTCCAGCATGTCGCAGTACAACTC
GCCAAAACCCAAGCGAACCTCCACATAAACCCCCTATTATACTCCCAGCC
TTAAACAAAGTGTATCTATTCCCCCCTGACAAGCCCACTCCCATACAGNN
NNNNNNNNNNNNNNNNAACACAGAGTTCGAAGCCTGCCAGGCCTTCGACA
GACCACCTAGAAAATACCTCTCAGACACACCTACCTACCCTTGGCTCCCC
GTCCCCAATCCTGAAATAAAGGTCAGCTTTAAGCTCGGTTTCAAATCTTA
CAAGGCCGTGGGAGTTTCACTGGTCGGTGTCTACCTCTTAAGGTCACTAA
GCACTCCGAGCGTCAGCGAGGAGTGCGACCCTTCCCCCTGGTGCAACGCC
CTCGGCGGCCGCGCGCTACGCCTTCGGCTGCGCGCGGCACCTCGGACCCC
CGCTCGTGCTGACGCGCTCGCGCGCGTCAGACCACTTCGGGCTCGCGGGG
GTCGGGAATTTTGCTAAACAGACTCCGAGTTGCCATTGGACACTGTAGCT
GTGAATCAGTAACGAAAGTGAGTGGGGCCAGACTTCGCCATAAGGCCTTT
ATCTTCTTGCCATTGGTCCGTGTAGGGGGTCGCCATAGGCTTCGGGTTCG
GTTTTAGGCCTTCCGGACTACAAAAATGGCGGATTTAGTGACGTCACGGC
CGCCATTTTAAGTAGGTGCCGTCCAGGACTGCTGTTCCGGGTCACAGGGC
ATCCTCGGCGGAACTTACACAAAATGGCGGTCAAAAACATCCGGGTCAAA
GGTCGCAGCTACGTCATAAGTCACGTGCAGGGGTCCTGCTGCGTCATATG
CGG (SEQ ID NO: 845)
Annotations:

| Putative Domain | Base range |
|---|---|
| TATA Box | 50-55 |
| Initiator Element | 68-83 |
| Transcriptional Start Site | 78 |
| 5′ UTR Conserved Domain | 138-208 |
| ORF2 | 305-691 |
| ORF2/2 | 305-687; 2422-2878 |
| ORF2/3 | 305-687; 2564-3317 |
| ORF2t/3 | 305-360; 2564-3317 |
| ORF1 | 556-2904 |
| ORF1/1 | 556-687; 2422-2904 |
| ORF1/2 | 556-687; 2564-2878 |
| Three open-reading frame region | 2626-2846 |
| Poly(A) Signal | 3316-3319 |

TABLE A3
Novel Anellovirus amino acid sequences (Alphatorquevirus, Clade 4)
TTV-RTx3 (Alphatorquevirus Clade 4)

| | |
|---|---|
| ORF2 | MSSTWRVPRGDVSARELCWYRSVRESHDAFCGCRDPVFHLSRLAARSNHQ |
| | GPPTPPTDERPSASTPVRRLLPLPSYPGEGPQARWPGGDGEGAGGARGGAG |
| | DGGARAGEEEYRPEDLDELFDAIEQEQ (SEQ ID NO: 846) |
| ORF2/2 | MSSTWRVPRGDVSARELCWYRSVRESHDAFCGCRDPVFHLSRLAARSNHQ |
| | GPPTPPTDERPSASTPVRRLLPLPSYPGEGPQARWPGGDGEGAGGARGGAG |
| | DGGARAGEEEYRPEDLDELFDAIEQEQSSETRAKTPKYPDPVDSLAKYKSLT |
| | RNSLRRRGSSTRSTRDEECLLQELSNVCSSNQYLASMLLHHSGSRSSFPPQSS |
| | SEREKMQKAAQVHHPRDRDSGRKRPTRRKRSPRRGRRRRRGSSSSESSESSE |
| | SSTSNSSMSQYNSPKPKRTST (SEQ ID NO: 847) |
| ORF2/3 | MSSTWRVPRGDVSARELCWYRSVRESHDAFCGCRDPVFHLSRLAARSNHQ |
| | GPPTPPTDERPSASTPVRRLLPLPSYPGEGPQARWPGGDGEGAGGARGGAG |
| | DGGARAGEEEYRPEDLDELFDAIEQEQSYQTSAQATNTWRVCSYTTQGPAP |
| | LSLLRVPARGRRCRKRLRFTTQETATLAGRGQPDANGVLGGAGGDDEGAP |
| | RAKAQRAASPQPPTPACRSTTRQNPSEPPHKPPIILPALNKVYLFPPDKPTPIQ |
| | XXXXXXNTEFEACQAFDRPPRKYLSDTPTYPWLPVPNPEIKVSFKLGFKSYK |
| | AVGVSLVGVYLLRSLSTPSVSEECDPSPWCNALGGRALRLRLRAAPRTPAR |
| | ADALARVRPLRARGGREFC (SEQ ID NO: 848) |
| ORF2t/3 | MSSTWRVPRGDVSARELCWSYQTSAQATNTWRVCSYTTQGPAPLSLLRVP |
| | ARGRRCRKRLRFTTQETATLAGRGQPDANGVLGGAGGDDEGAPRAKAQR |
| | AASPQPPTPACRSTTRQNPSEPPHKPPIILPALNKVYLFPPDKPTPIQXXXXXX |
| | NTEFEACQAFDRPPRKYLSDTPTYPWLPVPNPEIKVSFKLGFKSYKAVGVSL |
| | VGVYLLRSLSTPSVSEECDPSPWCNALGGRALRLRLRAAPRTPARADALAR |
| | VRPLRARGGREFC (SEQ ID NO: 849) |
| ORF1 | MAWWGWRRRWWRPRRRWRWRRPRRRRRVPARRPRRAVRRYRTRTVRR |
| | RRRGWRRGYRRRYRLRRYRRRGRRRKKIVLTQWNPQTVRKCFIRGLMPVL |
| | WAGMGTGGHNYAVRSDDFVVDRGFGGSFATETFSLRVLFDQYQRGFNRW |
| | SHTNEDLDLARYTGCKWTFYRHQDTDFIVYFTNNPPMKTNQHTAPLTTPG |
| | MLMRSKYKILVPSFKTRPKGRKTVSVRVRPPKLFQDKWYTQQDLCPVPLVQ |
| | LNVTAADFTHPFGSPLTDTPCIRFQVLGNLYNKCLNIDLPQFDEDGEILTSTP |
| | YNRENKEDLKKLYKTLFVDEHAGNYWQTFLTNTMVKSHIDANQAKTYDQ |
| | EKTAAEQGKDPFPTNPPKDQFTTWNKKLVDPRDSNFLFATYHPKNIKKAIKT |
| | MRDNNFALTTGKNDIYGDYTAAYTRNTHMLDYYLGFYSPIFLSSGRSNTEF |
| | WTAYRDIVYNPLLDKGTGNMIWFQYHTKTDNIYKKPECHWEILDMPLWAL |
| | CNGYVEYLESQIKYGDILVEGKVLIRCPYTKPALVDPNNSLAGYVVFNTTFG |
| | QGKWIDGKGYIPLHERSKWYVMLRYQTDVLHDIVTCGPWQYRDDNKNSQ |
| | LIAKYRFKFYWGGNMVHSQVIRNPCKDTQVSGPRRQPREVQVVDPQLITPP |
| | WVLHSFDQRRGMFTAGAIKRLLKQPIPGEYAPTPLRVPLLFPSSEFQREGED |
| | AESGSGSPPKRPRLWQEEANQTQTESSEGPAETTRELLERKLREQRVLNLQL |
| | QHVAVQLAKTQANLHINPLLYSQP (SEQ ID NO: 850) |
| ORF1/1 | MAWWGWRRRWWRPRRRWRWRRPRRRRRVPARRPRRAVRRYRTRTVIRN |
| | PCKDTQVSGPRRQPREVQVVDPQLITPPWVLHSFDQRRGMFTAGAIKRLLK |
| | QPIPGEYAPTPLRVPLLFPSSEFQREGEDAESGSGSPPKRPRLWQEEANQTQT |
| | ESSEGPAETTRELLERKLREQRVLNLQLQHVAVQLAKTQANLHINPLLYSQP |
| | (SEQ ID NO: 852) |
| ORF1/2 | MAWWGWRRRWWRPRRRWRWRRPRRRRRVPARRPRRAVRRYRTRTELSN |
| | VCSSNQYLASMLLHHSGSRSSFPPQSSSEREKMQKAAQVHHPRDRDSGRKR |
| | PTRRKRSPRRGRRRRRGSSSSESSESSESSTSNSSMSQYNSPKPKRTST (SEQ |
| | ID NO: 853) |

TABLE N4
Novel Anellovirus nucleic acid sequence (Alphatorquevirus)

| | |
|---|---|
| Name | TTV-RTx4 |
| Genus/Clade | Alphatorquevirus, Clade 4 |
| Accession Number | SRR3481579 |

Full Sequence: 3742 bp
10 20 30 40 50 60
| | | | | |
AAAGTGCTACGTCACTAACCACGTGACACCCACAGGCCAACCGAATGCTA
TGTCGTGCACTTCCTGGGCCGGGTCTACGTCCTCATATAACTACCTGCAC
TTCCGAATGGCTGAGTTTTCCACGCCCGTCCGCAGCGGTGAAGCCACGGA
GGGAGATCAGCGCGTCCCGAGGGCGGGTGCCGAAGGTGAGTTTACACACC
GAAGTCAAGGGGCAATTCGGGCTCGGGACTGGCCGGGCTATGGGCAAGGC
TCTGAAAAAAGCATGTTTATTGGCAGGCATTACAGAAAGAAAAGGGCGCT
GCCACTGTGTGCTGTGCGATCAACAAAGAAGGCTTGCAAACTACTAATAG
TAATGTGGACCCCACCTCGCAATGACCAACAGTACCTTAACTGGCAATGG
TACTCAAGTATACTTAGCTCCCACGCTGCTATGTGCGGGTGTCCCGACGT
TGTTGCTCATTTTAATCATCTTGCTTCTGTGCTTCGCGCCCCGCAAAATC
CACCCCCACCCGGTCCCCAGCGAAACCTGCCCCTCCGACGGCTGCCGGCT
CTCCCGGCTGCGCCAGAGGCGCCCGGAGATAGAGCACCATGGCCTATGGC
TGGTGGCGCCGGAGGAGAAGACGGTGGCGCAGGTGGAGACGCAGACCATG
GAGGCGCCGCTGGAGGACCAGAAGACGCAGACCTGTTAGACGCCGTGGCC
GCCGCAGAAACGTAAGGAGACGCCGCAGAGGAGGGAGGTGGAGGAGGAGG
TACAGGAGATGGAAAAGAAAGGGCAGACGCAGAAAAAAAGCTAAAATAAT
AATAAGACAATGGCAACCTAACTACAGAAGGAGATGTAACATAGTAGGCT
ATATTCCTGTACTGATATGTGGCGAAAATACTGTCAGCAGAAACTATGCC
ACACACTCAGACGATACTAACTACCCAGGACCCTTTGGGGGGGGTATGAC
TACAGACAAATTTACCTTAAGAATTCTGTATGACGAGTACAAAAGGTTTA
TGAACTATTGGACAGCATCTAATGAAGACCTAGACCTCTGTAGATATCTA
GGAGTAAACCTGTACTTTTTTAGACACCCAGAAGTAGACTTTATTATAAA
AATAAATACCATGCCCCCTTTTCTAGACACAGAACTAACAGCTCCTAGCA
TACACCCAGGAATGCTAGCCTTAGACAAAAGAGCAAGATGGATACCTAGC
TTAAAATCTAGACCAGGAAAAAAACACTATATTAAAATAAGAGTAGGGGC
GCCTAAAATGTTCACAGATAAATGGTACCCCCAAACAGATCTTTGTGACA
TGGTGCTGCTAACTGTCTATGCAACCGCAGCGGATATGCAATATCCGTTC
GGCTCACCACTAACTGACTCTGTGGTTGTGAACTTCCAGGTTCTGCAATC
CATGTATGATGAAACCATTAGCATATTACCAGATCAAAAGGAGAAAAGAA
TAACGCTGCTCACTAGTATAGCCTTTTATAACACCACACAAACTATAGCC
CAATTAAAGCCATTTATAGATGCAGGCAATATGACTTCAACTACAACAGC
AACAACATGGGGATCATACATAAACACAACCAAATTTAATACAGCAGCCA
CTACAACATACACATACCCAGGCAGTACTACAACTACAGTAACTATGTTA
ACTTGTAATGACTCCTGGTACAGAGGAACAGTATATAACGACCAAATTAA
AAATTTACCAAAGGAAGCAGCTCAATTATACTTAAAAGCAACAAAAACCT
TACTAGGAAACACCTTCACAAATGACGACCACACACTAGAATACCATGGA
GGACTGTACAGCTCAATTTGGCTGTCCCCCGGCAGATCTTACTTTGAAAC
ACCAGGAGCATACACAGACATAAAATACAACCCATTTACAGACAGAGGAG
AAGGAAACATGCTATGGATAGACTGGCTAAGCAAAAAAAATATGAACTAT
GACAAACTACAAAGTAAATGTTTAATATCAGACCTACCTTTATGGGCAGC
AGCATATGGATATTTAGAATTTTGTGCAAAAAGTACAGGAGACCAAAATA
TACACATGAATGCCAGACTACTAATAAGAAGTCCCTTTACAGACCCCCAA
CTACTAGTACACACAAACCCCACAAAAGGCTTTGTTCCCTACTCTTTAAA
CTTTGGAAATGGTAAAATGCCAGGAGGTAGTAGTAATGTTCCTATTAGAA
TGAGAGCTAAATGGTATCCAACATTGTTTCACCAGCAAGAAGTACTAGAG
GCCTTAGCACAGTCAGGCCCCTTTGCATACCACTCAGACATTAAAAAAGT
ATCTCTGGGTATGAAATACCGTTTTAAGTGGATCTGGGGTGGAAACCCCG
TTCGCCAACAGGTTGTTAGAAATCCCTGCAAAGACTCCCACTCCTCGGTC
AATAGAGTCCCTAGAAGCTTACAAATCGTTGACCCGAAATACAACTCACC
GGAACTCACATTCCATACGTGGGACTTCAGACGTGGCCTCTTTGGCCAGA
AAGCTATTGAGAGAATGCAACAACAACCAACAACTACTGACATTTTTTCA
GCAGGCCGCAAGAGACCCAGGAGGGACACCGAGGTGTACCACTCCAGCCA
AGAAGGGGAGCAAAAAGAAAGCTTACTTTTCCCCCCAGTCAAGCTCCTCA
GACGAGTCCCCCCGTGGGAAGACTCGCAGCAGGAGGAAAGCGGGTCGCAA
AGCTCAGAGGAAGAGACGCAGACCGTCTCCCAGCAGCTCAAGCAGCAGCT
GCAGCAACAGCGAATCCTGGGAGTCAAACTCATACTCCTGTTCAACCAAG
TCCAAAAAATCCAACAAAATCAAGATATCAACCCTACCTTGTTACCAAGG
GGGGGGGATCTAGCATCCTTATTTCAAATAGCACCATAAACATGTTTGGA
GACCCCAAACCTTACAACCCTTCCAGTAATGACTGGAAAGAGGAGTATGA
GGCCTGTAGAATATGGGACAGACCCCCAAGAGGCAATCTAAGAGACACCC
CCTTTTACCCCTGGGCCCCCAAAGAAAACCAGTACCGTGTAAACTTTAAA
CTTGGATTTCAATAAAGCTAGGCCGTGGGACTTTCACTTGTCGGTGTCTG
CTTATAAAAGTAACCAAGCACTCCGAGCGAAGCGAGGAGTGCGACCCTTG
GGGGCTCAACGACTTCGGAGCCGCGCGTTAAGCCTTCGGCTGCGCGCGGC
ACCTCAGACCCCCGCTCGTGCTGACACGCTTGCGCGTGTCAGACCACTTC
GGGCTCGCGGGGGTCGGGAAATTTATTAAACAGACTCCGAGTTGCCATTG
GACACAGTAGTCTATGAACAGCAACGAAAGTGAGTGGGGCCAGACTTCGC
CATAAGGCCTTTATCTTCTTGCCATTTGTCAGTATAGAGGGTCGCCATAG
GCTTCGGTCTCCATTTTAACCTGTAAAAACTACCAAAATGGCCGTTCCAG
TGACGTGACAGCCGCCATTTTAAGTAGCTGACGTCAAGGATTGACGTAAA
GGTTAAAGGTCATCCTCGGCGGAAGCTACACAAAATGGTGGACAACATCT
TCCGGGTCAAAGGTCGTGCACACGTCAAAAGTCACGTGGTGGGGACCCGC
TGTAACCCGGAAGTAGGCCCCGTCACGTGATTTGTCACGTGTGTACACGT
CACAGCCGCCATTTTGTTTTACAAAATGGCTGACTTCCTTCCTCTTTTTT
GAAAAAAGGCGCCAAAAAAGGCTCCGCCCCCCGGCCCCCCCC (SEQ ID NO: 854)
Annotations:

| Putative Domain | Base range |
|---|---|
| TATA Box | 86-90 |
| Initiator Element | 104-119 |
| Transcriptional Start Site | 114 |
| 5′ UTR Conserved Domain | 174-244 |
| ORF2 | 353-715 |
| ORF2/2 | 353-711; 2362-2863 |
| ORF2/3 | 353-711; 2555-3065 |
| ORF2t/3 | 353-432; 2555-3065 |
| ORF1 | 589-2889 |
| ORF1/1 | 589-711; 2362-2889 |
| ORF1/2 | 589-711; 2555-2863 |
| Three open-reading frame region | 2555-2863 |
| Poly(A) Signal | 3062-3066 |
| GC-rich region, or a portion thereof | 3720-3742 |

TABLE A4
Novel Anellovirus amino acid sequences (Alphatorquevirus, Clade 4)
TTV-RTx4 (Alphatorquevirus Clade 4)

| ORF2 | MWTPPRNDQQYLNWQWYSSILSSHAAMCGCPDVVAHFNHLASVLRAPQN |
|---|---|
| | PPPPGPQRNLPLRRLPALPAAPEAPGDRAPWPMAGGAGGEDGGAGGDADH |
| | GGAAGGPEDADLLDAVAAAET (SEQ ID NO: 855) |
| ORF2/2 | MWTPPRNDQQYLNWQWYSSILSSHAAMCGCPDVVAHFNHLASVLRAPQN |
| | PPPPGPQRNLPLRRLPALPAAPEAPGDRAPWPMAGGAGGEDGGAGGDADH |
| | GGAAGGPEDADLLDAVAAAETLLEIPAKTPTPRSIESLEAYKSLTRNTTHRN |
| | SHSIRGTSDVASLARKLLRECNNNQQLLTFFQQAARDPGGTPRCTTPAKKGS |
| | KKKAYFSPQSSSSDESPRGKTRSRRKAGRKAQRKRRRPSPSSSSSSCSNSESW |
| | ESNSYSCSTKSKKSNKIKISTLPCYQGGGI (SEQ ID NO: 856) |
| ORF2/3 | MWTPPRNDQQYLNWQWYSSILSSHAAMCGCPDVVAHFNHLASVLRAPQN |
| | PPPPGPQRNLPLRRLPALPAAPEAPGDRAPWPMAGGAGGEDGGAGGDADH |
| | GGAAGGPEDADLLDAVAAAETPQETQEGHRGVPLQPRRGAKRKLTFPPSQ |
| | APQTSPPVGRLAAGGKRVAKLRGRDADRLPAAQAAAAATANPGSQTHTPV |
| | QPSPKNPTKSRYQPYLVTKGGGSSILISNSTINMFGDPKPYNPSSNDWKEEYE |
| | ACRIWDRPPRGNLRDTPFYPWAPKENQYRVNFKLGFQ (SEQ ID NO: 857) |
| ORF2t/3 | MWTPPRNDQQYLNWQWYSSILSSHAAMPQETQEGHRGVPLQPRRGAKRK |
| | LTFPPSQAPQTSPPVGRLAAGGKRVAKLRGRDADRLPAAQAAAAATANPGS |
| | QTHTPVQPSPKNPTKSRYQPYLVTKGGGSSILISNSTINMFGDPKPYNPSSND |
| | WKEEYEACRIWDRPPRGNLRDTPFYPWAPKENQYRVNFKLGFQ (SEQ ID |
| | NO: 858) |
| ORF1 | MAYGWWRRRRRRWRRWRRRPWRRRWRTRRRRPVRRRGRRRNVRRRRR |
| | GGRWRRRYRRWKRKGRRRKKAKIIIRQWQPNYRRRCNIVGYIPVLICGENT |
| | VSRNYATHSDDTNYPGPFGGGMTTDKFTLRILYDEYKRFMNYWTASNEDL |
| | DLCRYLGVNLYFFRHPEVDFIIKINTMPPFLDTELTAPSIHPGMLALDKRARW |
| | IPSLKSRPGKKHYIKIRVGAPKMFTDKWYPQTDLCDMVLLTVYATAADMQ |
| | YPFGSPLTDSVVVNFQVLQSMYDETISILPDQKEKRITLLTSIAFYNTTQTIAQ |
| | LKPFIDAGNMTSTTTATTWGSYINTTKFNTAATTTYTYPGSTTTTVTMLTCN |
| | DSWYRGTVYNDQIKNLPKEAAQLYLKATKTLLGNTFINDDHTLEYHGGLY |
| | SSIWLSPGRSYFETPGAYTDIKYNPFTDRGEGNMLWIDWLSKKNMNYDKLQ |
| | SKCLISDLPLWAAAYGYLEFCAKSTGDQNIHMNARLLIRSPFTDPQLLVHTN |
| | PTKGFVPYSLNFGNGKMPGGSSNVPIRMRAKWYPTLFHQQEVLEALAQSGP |
| | FAYHSDIKKVSLGMKYRFKWIWGGNPVRQQVVRNPCKDSHSSVNRVPRSL |
| | QIVDPKYNSPELTFHTWDFRRGLFGQKAIERMQQQPTTTDIFSAGRKRPRRD |
| | TEVYHSSQEGEQKESLLFPPVKLLRRVPPWEDSQQEESGSQSSEEETQTVSQ |
| | QLKQQLQQQRILGVKLILLFNQVQKIQQNQDINPTLLPRGGDLASLFQIAP |
| | (SEQ ID NO: 859) |
| ORF1/1 | MAYGWWRRRRRRWRRWRRRPWRRRWRTRRRRPVRRRGRRRNVVRNPC |
| | KDSHSSVNRVPRSLQIVDPKYNSPELTFHTWDFRRGLFGQKAIERMQQQPTT |
| | TDIFSAGRKRPRRDTEVYHSSQEGEQKESLLFPPVKLLRRVPPWEDSQQEES |
| | GSQSSEEETQTVSQQLKQQLQQQRILGVKLILLFNQVQKIQQNQDINPTLLPR |
| | GGDLASLFQIAP (SEQ ID NO: 860) |
| ORF1/2 | MAYGWWRRRRRRWRRWRRRPWRRRWRTRRRRPVRRRGRRRNAARDPG |
| | GTPRCTTPAKKGSKKKAYFSPQSSSSDESPRGKTRSRRKAGRKAQRKRRRPS |
| | PSSSSSSCSNSESWESNSYSCSTKSKKSNKIKISTLPCYQGGGI (SEQ ID NO: |
| | 861) |

TABLE N5
Novel Anellovirus nucleic acid sequence (Alphatorquevirus)

| | |
|---|---|
| Name | TTV-RTx5b |
| Genus/Clade | Alphatorquevirus, Clade 5 |
| Accession Number | SRR3481639 |

Full Sequence: 3553 bp
10 20 30 40 50 60
| | | | | |
ATACCTCATCATATAAAGCGGCGCACTTCCGAATGGCTGAGTTTTCCACG
CCCGTCCGCAGCGAGATCGCGACGGAGGAGCGATCGAGCGTCCCGAGGGC
GGGTGCCGGAGGTGAGTTTACACACCGCAGTCAAGGGGCAATTCGGGCTC
GGGACTGGCCGGGCTATGGGGCAAGACTCTTAAAAAAGCCATGTTTCTCG
GTAAACTTTACAGAAAGAAAAGGGCACTGTCACTGCTACGCGTGCGAGCT
CCAGAGGCGAAACCACCTGCTATGAGTTGGAGACCCCCGGTGCACAACCC
CAATGGGATCGAGAGAAACCTGTGGGAGGCATTCTTTCGCATGCATGCTT
CAGCTTGTGGTTGTGGCGATCTTGTTGGCCATCTTACTGTACTGGCTGGT
CGGTATGGTGCTCCTCCTCGTCCCCCGGCCCCCGGCGCTCCCAGACCACC
GCTGATACGCCAGCTGGCCCTTCCGGCGCCCCCCGCCGATCCTCAACAGG
CTAACCCACAATGGCCTGGTGGGGACGGTGGAGAAGATGGCGCTGGAGGC
CCCGCCGCTGGCGGCGCCGTCGCAGACGCCGAGTACCAAGAAGACGAGCT
CAACGCCCTGTTCGACGCCGTCGAGCAAGAAGAGTAAGGAGGAGGCGATG
GGGGAGGCGGAGGTGGAGACGGGGGTACAGACGCAGACTGAGACTAAGAC
GCAGACGCAGACGAAAGCGAAAGATAGTACTAACTCAGTGGAATCCCGCC
AAAGTGCGGAGGTGTACTATTAAGGGAGTTCTGCCCATGATCCTGTGCGG
GGCCGGGCGCTCGGGGITTAACTACGGACTGCACAGCGACGACTACACTG
TACAGAAGCCCCTTGGCCAGAACCCCCACGGGGGCGGCATGAGTACAGTG
ACTTTTAGCCTACAGGTGCTCTATGACCAGTACCAGAGGTTTATGAACAA
GTGGTCGTACTCCAACGACCAGCTAGACCTCGCCAGGTACTTTGGCTGCA
CCTTCTGGTTCTACAGACACCCAGAGGTGGACTTTGTAGCTCAGTTTGAC
AACGTTCCCCCCATGAAAATGGACGAGAACACAGCCCCAAACACTCATCC
CTCTTTCTTACTACAGAACAAACACAAGGTTAAAATTCCCAGCTTTAAAA
CAAAGCCTTTTGGTAAAAAAAGAGTTAGAGTTACAGTAGGGCCCCCCAAA
CTGTTTGAAGATAAGTGGTACAGCCAACATGACTTGTGTAAGGTGCCCCT
AGTCAGTTGGCGGTTAACCGCAGCTGACTTCAGGTTTCCGTTCTGCTCAC
CACAAACTGACAACCCTTGCTACACCTTCCAGGTATTGCATGAAGAGTAT
TACCCAGTAATAGGCACTTCTGCTTTAGAAAACGGCAGTAACTACAATAG
CTCAGCTATAACAGCCTTAGAAAAATTCTTATATGAAAAATGCACACACT
ATCAAACATTTGCCACAGACACCAGACTTAATCCTCAGCGACCAGTGTCA
TCTACAAATGCAAACAAAACATACACCCCCTCAGGCTCCCAAGAAACAAT
AGTGTGGGGGCAGTCAGATTTTAATTTATTTAAAAAGCACACAGACAGCA
ACTATGGCTACTGCACCTACTGTCCTACCAATGACTTAGCTACAAAAATT
AAAAAGTACAGAGACAAAAGATTCGACTGGCTAACAAACATGCCAGTAAC
AAACACCTGCCACATAAATGCCACCTTCGCCCGAGGCAAAATTAAAGAAT
GGGAGTACCACCTAGGGTGGTTCTCAAACATCTTTATAGGCAACCTGAGA
CACAACCTAGCATTCCGGGCCGCATACATAGACATCACCTANACAGACAA
GGGAGAAGGCAACATTATCTGGTTCCAGTACCTCACTAAACCCACCACAG
AGTACATAGAAGCCCAAGCAAAGTGCTCCATCACAAACATACCCCTGTAT
GCTGCTTTTTATGGCTACGAAGACTACCTCCAGAGAACACTAGGCCCCTA
CCAAGATGTAGAAACCCTAGGTATAATCTGTGTTAAATGTCCCTACACAG
ATCCCCCTCTAGITCACAAGTCTACAGATAAAAAGAACTGGGGCTACGTG
TTCTACGACGTGCACTTTGGCAACGGAAAGACCCCAGAGGGACTGGGCCA
GGTGCACCCTTACTGGATGCAGAGGTGGAGACCCTACGTACAGTTTCAGA
AAGACACTATGAACAAAATAGCCAGGACGGGACCGTTCAGCTACAGAGAC
GAGACGCCTTCCATCACCCTGACCGCCGGGTACAAGTTTCATTTTAACTG
GGGGGGCGACTCTATATTTCCACAGATTATTAAAAACCCCTGCCCAGACA
GCGGGGTACGACCTTCATCCAGTAGAGAGCGTCGCTCAGTACAAGTCGTT
AGCCCGCTCACAATGGGGCCAGAGTACATATTCCACCGGTGGGACTGGCG
ACGGGGGTTCTTTAATCAAAAAGCTCTCAAAAGAATGCTTGAAAAATCAA
TTAATGATGGAGAGTATCCAACAGGCCCAAAGGTCCCTCGATGGTTTCCC
CCACTCGACAACCAAGAGCAAGAAGGCGCCTCAGGTTCAGAGGAGACAAG
GTCGCAGTCCTCGCAAGAAGAAGCCGCTCAAGAAGCCCTCCAAGAAGTCC
AAGAGGCGTCGCTACAGCAGCACCTCCTCCAGCAGTACCGAGAGCAGCGA
CGGATCGGAAAGCAACTCCAACTCGTCATGCTGCAGCTCACCAAGACGCA
GAGCAACCTGCACATAAACCCCCGTGTTCTTGGCCATGCATAAATAAAGT
CTACATGTTTCCCCCCGACAAGCCCATGCCCATACACGGGTACCACGGGT
GGGAGACGGAGTACCAGGCCTGCAAGGCCTTCAACAGGCCCCCCAGAAAC
TACCTTTCAGACAAACCCATCTACCCTTGGCTCCCTCGCCCCGAACCCGA
AATAATAGTGAGCTTTAGGTTCGGTTTCAAATAAACAAGGCCGCAAATAA
ACAAGGCCGTGGGAGTTTCACTGGTCGGTGTCTACCTCTTAAGGTCACTA
AGCACTCCGAGCGTTAGCGAGGAGTGCGACCCTTCCCCCTGGTGCCACGC
CCTCGGCGGCCGCGCGCTACGCCTNNNNNNNNNNNNNNNNNNNNNNNNNN
NNNNNNNNNNNNNNNNNNNNNNNNNNTGAATCAGTAACGAAAGTGAGTGG
GGCCAGACTTCGCCATAAGGCCTTTATCTTCTTGCCATTGGTCCGTGTGG
GGAGTCGCCATAGGCTTCGGGCTCGGTTTTAGGCCTTCCGGACTACAAAA
ACCGCCATTTTAGTGACGTCACGGCGGCCATTTTAAGTAAGCATGGCGGG
CGGTGACGTACAAGTTGAAAGGTCACCGCGCTTCCGTGTTTACTCAAAAT
GGTGGCCAACTGCTTCCGGGTCAAAGGTCGGCGGCCACGTCATAAGTCAC
GTGGGAGGGCTGCGTCACAAACACGGAAGTGGCTGTCCCACGTGACTTGT
CACGTGATTGCTACGTCACGGCCGCCATTTTAGTTCACAAAATGGCGGAC
TTC (SEQ ID NO: 862)
Annotations:

| Putative Domain | Base range |
|---|---|
| TATA Box | 12-17 |
| Initiator Element | 30-45 |
| Transcriptional Start Site | 40 |
| 5′ UTR Conserved Domain | 100-171 |
| ORF2 | 272-637 |
| ORF2/2 | 272-633; 2326-2767 |
| ORF2/3 | 272-633; 2525-2984 |
| ORF2t/3 | 272-633; 2525-2984 |
| ORF1 | 511-2793 |
| ORF1/1 | 511-711; 2326-2793 |
| ORF1/2 | 511-711; 2525-2767 |
| Three open-reading frame region | 2525-2767 |
| Poly(A) Signal | 2981-2985 |
| Unknown sequence | 3125-3176 |

*Note:
Modifications made to maintain reading frames:
-“C” inserted into ORF2 430
-“N” inserted into ORF1 1842

TABLE A5
Novel Anellovirus amino acid sequences (Alphatorquevirus, Clade 5)
TTV-RTx5b (Alphatorquevirus Clade 5)

| | |
|---|---|
| ORF2 | MSWRPPVHNPNGIERNLWEAFFRMHASACGCGDLVGHLTVLAGRYGAPPR |
| | PPAPGAPRPPLIRQLALPAPPADPQQANPQWPGGDGGEDGAGGPAAGGAVA |
| | DAEYQEDELNALFDAVEQEE (SEQ ID NO: 863) |
| ORF2/2 | MSWRPPVHNPNGIERNLWEAFFRMHASACGCGDLVGHLTVLAGRYGAPPR |
| | PPAPGAPRPPLIRQLALPAPPADPQQANPQWPGGDGGEDGAGGPAAGGAVA |
| | DAEYQEDELNALFDAVEQEELLKTPAQTAGYDLHPVESVAQYKSLARSQW |
| | GQSTYSTGGTGDGGSLIKKLSKECLKNQLMMESIQQAQRSLDGFPHSTTKSK |
| | KAPQVQRRQGRSPRKKKPLKKPSKKSKRRRYSSTSSSSTESSDGSESNSNSSC |
| | CSSPRRRATCT (SEQ ID NO: 864) |
| ORF2/3 | MSWRPPVHNPNGIERNLWEAFFRMHASACGCGDLVGHLTVLAGRYGAPPR |
| | PPAPGAPRPPLIRQLALPAPPADPQQANPQWPGGDGGEDGAGGPAAGGAVA |
| | DAEYQEDELNALFDAVEQEEPKGPSMVSPTRQPRARRRLRFRGDKVAVLAR |
| | RSRSRSPPRSPRGVATAAPPPAVPRAATDRKATPTRHAAAHQDAEQPAHKP |
| | PCSWPCINKVYMFPPDKPMPIHGYHGWETEYQACKAFNRPPRNYLSDKPIY |
| | PWLPRPEPEIIVSFRFGFK (SEQ ID NO: 865) |
| ORF2t/3 | MSWRPPVHNPNGIERNLWEAFFRMHASACGCGDLVGHLTVLAGRPKGPSM |
| | VSPTRQPRARRRLRFRGDKVAVLARRSRSRSPPRSPRGVATAAPPPAVPRAA |
| | TDRKATPTRHAAAHQDAEQPAHKPPCSWPCINKVYMFPPDKPMPIHGYHG |
| | WETEYQACKAFNRPPRNYLSDKPIYPWLPRPEPEIIVSFRFGFK (SEQ ID NO: |
| | 866) |
| ORF1 | MAWWGRWRRWRWRPRRWRRRRRRRVPRRRAQRPVRRRRARRVRRRRW |
| | GRRRWRRGYRRRLRLRRRRRRKRKIVLTQWNPAKVRRCTIKGVLPMILCG |
| | AGRSGFNYGLHSDDYTVQKPLGQNPHGGGMSTVTFSLQVLYDQYQRFMN |
| | KWSYSNDQLDLARYFGCTFWFYRHPEVDFVAQFDNVPPMKMDENTAPNT |
| | HPSFLLQNKHKVKIPSFKTKPFGKKRVRVTVGPPKLFEDKWYSQHDLCKVP |
| | LVSWRLTAADFRFPFCSPQTDNPCYTFQVLHEEYYPVIGTSALENGSNYNSS |
| | AITALEKFLYEKCTHYQTFATDTRLNPQRPVSSTNANKTYTPSGSQETIVWG |
| | QSDFNLFKKHTDSNYGYCTYCPTNDLATKIKKYRDKRFDWLTNMPVTNTC |
| | HINATFARGKIKEWEYHLGWFSNIFIGNLRHNLAFRAAYIDITXTDKGEGNII |
| | WFQYLTKPTTEYIEAQAKCSITNIPLYAAFYGYEDYLQRTLGPYQDVETLGII |
| | CVKCPYTDPPLVHKSTDKKNWGYVFYDVHFGNGKTPEGLGQVHPYWMQR |
| | WRPYVQFQKDTMNKIARTGPFSYRDETPSITLTAGYKFHFNWGGDSIFPQIIK |
| | NPCPDSGVRPSSSRERRSVQVVSPLTMGPEYIFHRWDWRRGFFNQKALKRM |
| | LEKSINDGEYPTGPKVPRWFPPLDNQEQEGASGSEETRSQSSQEEAAQEALQ |
| | EVQEASLQQHLLQQYREQRRIGKQLQLVMLQLTKTQSNLHINPRVLGHA |
| | (SEQ ID NO: 867) |
| ORF1/1 | MAWWGRWRRWRWRPRRWRRRRRRRVPRRRAQRPVRRRRARRIIKNPCPD |
| | SGVRPSSSRERRSVQVVSPLTMGPEYIFHRWDWRRGFFNQKALKRMLEKSI |
| | NDGEYPTGPKVPRWFPPLDNQEQEGASGSEETRSQSSQEEAAQEALQEVQE |
| | ASLQQHLLQQYREQRRIGKQLQLVMLQLTKTQSNLHINPRVLGHA (SEQ ID |
| | NO: 868) |
| ORF1/2 | MAWWGRWRRWRWRPRRWRRRRRRRVPRRRAQRPVRRRRARRAQRSLDG |
| | FPHSTTKSKKAPQVQRRQGRSPRKKKPLKKPSKKSKRRRYSSTSSSSTESSD |
| | GSESNSNSSCCSSPRRRATCT (SEQ ID NO: 869) |

TABLE N6
Novel Anellovirus nucleic acid sequence (Alphatorquevirus)

| | |
|---|---|
| Name | TTV-RTx6 |
| Genus/Clade | Alphatorquevirus, Clade 5 |
| Accession Number | SRR3438066 |

Full Sequence: 3896 bp
10 20 30 40 50 60
| | | | | |
TAAACTTCCTCTTTTAATAGGAAACCACAAAATTTGCATTGCCGACCACA
AACGCATATGCAAATTTACTTCCCCAAAAACTCAACCACAAAATTTGCAT
TGCCGCCCACAAACGTCTACTTTAACCACATCCTCTAACATGTTAGAAAC
TCCACCCAACTACTTCATTAGTATACAGCATCACAAGGGAGGAGCCAAAC
AACTATATAACCAAGTGTACTTCCGAATGGCTGAGTTTATGCCGCCAGAC
GGAGACGGGATCGCGACGGAGGAGCGATCGAGCGTCCCGAGGGCGGGTGC
CGGAGGTGAGTTTACACACCGCAGTCAAGGGGCAATTCGGGCTCGGGACT
GGCCGGGCTATGGGCAAGGCTCTTAAAAAAGCCATGTTTCTCGGTCGACC
TTACAGAAAGAAAAGGGCACTGTCACTGCTACGCGTGCGAGCTCCAGAGG
CGAAACCACCTGCTATGAGCTGGAGGCCCCCGGTGCACAACCCTAATGGG
ATCCAGAGAAACCTGTGGGAGGCATTCTTTCGCATGCATGCTGCAGCTTG
TGGTTGTGGCGATCTTGTTGGCCATATTACTGTACTGGCTGGTCGGTATG
GTGCTCCTCCTCGTCCCCCGGCCCCCGGGGCTCCCAGACCACCGCTGATA
CGCCAGCTGGCCCTTCCGGCGCCCCCCGCCGATCCTCAACAGGCTAACCC
ACAATGGCCTGGTGGGGACGGTGGAGAAGATGGCGCTGGAGGCCCCGCCG
CTGGCGGCGCCGTCGCAGACGCCGAGTACCAAGAAGACGAGCTCAACGCC
CTGTTCGACGCCGTCGAGCAAGAAGAGTAAGGAGGAGGCGATGGGGGAGG
CGGAGGTGGAGACGGGGGTACAGACGCAGACTAAGACTGAGACGCAGACG
CAGACGAAAGAAAATAAGACTGACTCAGTGGAACCCAGCCAAAGTCAGGA
GATGTACTATTAAGGGGGTGCTACCCATGATCTTATGCGGCGCCGGCCGC
TCGGGGTTTAACTATGGACTGCACAGCGACGACTACACGGTGCAGAAACC
CCTGGGGCAGAACCCCCACGGGGGCGGCATGAGCACAGTAACTTTTAGCC
TACAAGTACTATTTGACCAGTACCAGAGGTTTATGAACCGGTGGTCGTAC
TCCAACGACCAGCTAGACCTCGCCAGGTACTTTGGCTGCACCTTCTACTT
TTACAGACACCCTGAAATTGACTTTGTAGCTCAGTATGACAATGTACCCC
CAATGAAAATGGACGAGAACACGGCNCCTAACACTCACCCCTCTTTTCTA
CTACAAAACAAACGCAAAATTAAAATCCCCAGCTTTAAAACCAAGCCATT
TGGCAGAAAAAGAGTAAAAGTAACAGTGGGGCCCCCCAAACTGTTTGAAG
ATAAATGGTACAGCCAGCATGACTTGTGTAAGGTGCCCCTAGTCAGTTGG
CGGTTAACCGCATGTGACTTCAGGTTTCCGTTCTGCTCACCACTAACTGA
CAACCCTTGCTACACCTTCCAGGTATTGCATGAAAACTATTACCCAGTCA
TAGGCACTTCCTCTTTAGAAAACGGTACAAACTACAATAACACTGCTATA
ACTACCCTTGAGACATGGCTATATGGAAAATGCACACACTATCAAACATT
TGCCACAGACACCAGACTTAATCCACAGAGACCTGTATCTTCAAGTAATG
CAAATGAAACTTATACTCCTAGTGGTTCTAAAGAATCAATAATATGGGGA
CAGTCTGACTGGGCAAACTTTAAAAAGAACACAGACAGCAACTATGGCTA
CTGTTCCTACTGCCCCTCAAATGGCACTAACGGAACAGTAGATAAAATTA
AAAAATACAGAGACCAAAGATTTAGATGGCTTACAGAAATGCCAGTACCT
AACACCTGTCACATACATGCCACCTTCGCCCGAGGCACTATTAAATACTG
GGAGTACCACCTAGGCTGGTACTCAAACATATTTATTGGCAACCTCAGAC
ACAACTTAGCCTTCAGACCAGCCTACATAGACATTACCTACAATCCCATC
ACTGACAAAGGAGAGGGCAACATTATCTGGTTCCAGTACCTCACTAAGCC
CACCACAGAATACATAGAAACCCAGGCAAAATGCACCATTACTAACATTC
CCCTTTATGCTGCTTTCTATGGCTACGAAGACTACCTCCAGAGAACACTA
GGCCCCTACCAAGATGTAGAAACCCTAGGCATAATCTGTGTTAAATGTCC
CTACACAGATCCCCCTCTAGTTCACAAAGACAAAAGTAAAACCAACTGGG
GCTACGTATTCTACGACGCCCACTTTGGCAACGGAAAGACCCCAGAGGGA
CTAGGCCAAGTACACCCTTACTGGATGCAGAGATGGAGACCCTATGTACA
GTTTCAAAAAGACACCATGCACAAAATATCCAGAACGGGACCCTTCAGCT
ACAGAGACGACACGCCTTCCATCACCCTCACTGCCGAATACAAGTTTCGT
TTTAACTGGGGGGGCGACTCTATATTTCCACAGATTATTAAAAACCCCTG
CCCAGACACCGGGGTTCGACCTTCAACCGGTAGAGACCGTCGCTCAGTAC
AAGTCGTTAGCCCGCTCACAATGGGACCCCAGTTTATATTCCACTCATGG
GACTGGAGACGGGGGTTCTTTAATCAAAAAACTCTCAAAAGAATGCTTGA
AAAACCAGTTAATGATGGAGAATATCCAACAGGCCCAAAGGTGCCTCGAT
GGTTTCCCCCACTCGACAACCAAGAGCAAGAAGGCGTCTCAGATACAGAG
ACGACAACCTCGCAGTCCTCGCAAGAAGAAGCCGCTCAAGAAGCCCTCCA
AGAAGTCCAAGAGGCGTCGCTACAGCAGCACCTCCTCCAGCAGTACCGAG
AGCAGCGAAGAATCGGAAAGCAACTCCAACTCGTCATGCTCCAACTCACC
AAGACGCAGAGCAACCTGCACATAAATCCCCGTGTCCTTGGCCATGCATA
AATAAAGTGTACATGTTTCCCCCCGAAAAGCCAATGCCCATACACGGCTA
CCACGGGTGGGAGACAGAGTATCAGGCCTGCAAGGCCTTTGACAGGCCCC
CTAGAAACTACCTATCAGACAAACCCATCTACCCCTGGCTTCCCCGCTCC
CAACCAGAATTTAAAGTGAGTTTTAAGCTTGGCTGTCAATAAACAAGNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNNNNNNNNNNNNNNNNNNNNNNNNNNNNNNNNNNNNNNNNNNNNNN
NNNNNGTTTACACAAAATGGTGGCCAAGTCCTTCCGGGTGAAAGGTCGGC
GCCTACGTCATAAGTCACGTGGGGAGGGCTGCGTCACAACCAGGAAGCAA
TCCTCACCACGTGATTTGTCACGTGATCGCTACGTCACGGCCGCCATTTT
AGTTTACAAAATGGCGGACTTCCTTCCTCTTTTTCAAAAATAACGGCCCT
GCGGCGGCGCGCGCGCTGCGCGCGCGCGCCGGGGGCTGCCGCCCCA (SEQ ID NO: 870)
Annotations:

| Putative Domain | Base range |
|---|---|
| TATA Box | 206-210 |
| Initiator Element | 224-239 |
| Transcriptional Start Site | 234 |
| 5′ UTR Conserved Domain | 294-364 |
| ORF2 | 465-830 |
| ORF2/2 | 465-826; 2534-2975 |
| ORF2/3 | 465-826; 2721-3192 |
| ORF2t/3 | 465-595; 2721-3192 |
| ORF1 | 704-3001 |
| ORF1/1 | 704-826; 2534-3001 |
| ORF1/2 | 704-826; 2721-2975 |
| Three open-reading frame region | 2721-2975 |
| Poly(A) Signal | 3189-3193 |
| Unknown sequence | 3198-3655 |
| GC-rich region, or a portion thereof | 3844-3895 |

TABLE A6
Novel Anellovirus amino acid sequences (Alphatorquevirus, Clade 5)
TTV-RTx6 (Alphatorquevirus Clade 5)

| | |
|---|---|
| ORF2 | MSWRPPVHNPNGIQRNLWEAFFRMHAAACGCGDLVGHITVLAGRYGAPPR |
| | PPAPGAPRPPLIRQLALPAPPADPQQANPQWPGGDGGEDGAGGPAAGGAVA |
| | DAEYQEDELNALFDAVEQEE (SEQ ID NO: 871) |
| ORF2/2 | MSWRPPVHNPNGIQRNLWEAFFRMHAAACGCGDLVGHITVLAGRYGAPPR |
| | PPAPGAPRPPLIRQLALPAPPADPQQANPQWPGGDGGEDGAGGPAAGGAVA |
| | DAEYQEDELNALFDAVEQEELLKTPAQTPGFDLQPVETVAQYKSLARSQW |
| | DPSLYSTHGTGDGGSLIKKLSKECLKNQLMMENIQQAQRCLDGFPHSTTKS |
| | KKASQIQRRQPRSPRKKKPLKKPSKKSKRRRYSSTSSSSTESSEESESNSNSSC |
| | SNSPRRRATCT (SEQ ID NO: 872) |
| ORF2/3 | MSWRPPVHNPNGIQRNLWEAFFRMHAAACGCGDLVGHITVLAGRYGAPPR |
| | PPAPGAPRPPLIRQLALPAPPADPQQANPQWPGGDGGEDGAGGPAAGGAVA |
| | DAEYQEDELNALFDAVEQEEISNRPKGASMVSPTRQPRARRRLRYRDDNLA |
| | VLARRSRSRSPPRSPRGVATAAPPPAVPRAAKNRKATPTRHAPTHQDAEQP |
| | AHKSPCPWPCINKVYMFPPEKPMPIHGYHGWETEYQACKAFDRPPRNYLSD |
| | KPIYPWLPRSQPEFKVSFKLGCQ (SEQ ID NO: 873) |
| ORF2t/3 | MSWRPPVHNPNGIQRNLWEAFFRMHAAACGCGDLVGHITVLAGRISNRPK |
| | GASMVSPTRQPRARRRLRYRDDNLAVLARRSRSRSPPRSPRGVATAAPPPA |
| | VPRAAKNRKATPTRHAPTHQDAEQPAHKSPCPWPCINKVYMFPPEKPMPIH |
| | GYHGWETEYQACKAFDRPPRNYLSDKPIYPWLPRSQPEFKVSFKLGCQ |
| | (SEQ ID NO: 874) |
| ORF1 | MAWWGRWRRWRWRPRRWRRRRRRRVPRRRAQRPVRRRRARRVRRRRW |
| | GRRRWRRGYRRRLRLRRRRRRKKIRLTQWNPAKVRRCTIKGVLPMILCGA |
| | GRSGFNYGLHSDDYTVQKPLGQNPHGGGMSTVTFSLQVLFDQYQRFMNR |
| | WSYSNDQLDLARYFGCTFYFYRHPEIDFVAQYDNVPPMKMDENTAPNTHP |
| | SFLLQNKRKIKIPSFKTKPFGRKRVKVTVGPPKLFEDKWYSQHDLCKVPLVS |
| | WRLTACDFRFPFCSPLTDNPCYTFQVLHENYYPVIGTSSLENGTNYNNTAIT |
| | TLETWLYGKCTHYQTFATDTRLNPQRPVSSSNANETYTPSGSKESIIWGQSD |
| | WANFKKNTDSNYGYCSYCPSNGTNGTVDKIKKYRDQRFRWLTEMPVPNTC |
| | HIHATFARGTIKYWEYHLGWYSNIFIGNLRHNLAFRPAYIDITYNPITDKGEG |
| | NIIWFQYLTKPTTEYIETQAKCTITNIPLYAAFYGYEDYLQRTLGPYQDVETL |
| | GIICVKCPYTDPPLVHKDKSKTNWGYVFYDAHFGNGKTPEGLGQVHPYWM |
| | QRWRPYVQFQKDTMHKISRTGPFSYRDDTPSITLTAEYKFRFNWGGDSIFPQ |
| | IIKNPCPDTGVRPSTGRDRRSVQVVSPLTMGPQFIFHSWDWRRGFFNQKTLK |
| | RMLEKPVNDGEYPTGPKVPRWFPPLDNQEQEGVSDTETTTSQSSQEEAAQE |
| | ALQEVQEASLQQHLLQQYREQRRIGKQLQLVMLQLTKTQSNLHINPRVLGH |
| | A (SEQ ID NO: 875) |
| ORF1/1 | MAWWGRWRRWRWRPRRWRRRRRRRVPRRRAQRPVRRRRARRIIKNPCPD |
| | TGVRPSTGRDRRSVQVVSPLTMGPQFIFHSWDWRRGFFNQKTLKRMLEKPV |
| | NDGEYPTGPKVPRWFPPLDNQEQEGVSDTETTTSQSSQEEAAQEALQEVQE |
| | ASLQQHLLQQYREQRRIGKQLQLVMLQLTKTQSNLHINPRVLGHA (SEQ ID |
| | NO: 876) |
| ORF1/2 | MAWWGRWRRWRWRPRRWRRRRRRRVPRRRAQRPVRRRRARRNIQQAQR |
| | CLDGFPHSTTKSKKASQIQRRQPRSPRKKKPLKKPSKKSKRRRYSSTSSSSTE |
| | SSEESESNSNSSCSNSPRRRATCT (SEQ ID NO: 877) |

TABLE N7
Exemplary Anellovirus nucleic acid sequence (Alphatorquevirus, Clade 1)

| | |
|---|---|
| Name | TTV-CT30F |
| Genus/Clade | Alphatorquevirus, Clade 1 |
| Accession Number | AB064597.1 |

Full Sequence: 3570 bp
10 20 30 40 50 60
| | | | | |
ATTTTGTGCAGCCCGCCAATTCTCGTTCAAACAGGCCAATCAGGAGGCTC
TACGTACACTTCCTGGGGTGTGTCTTCGAAGAGTATATAAGCAGAGGCGG
TGACGAATGGTAGAGTTTTTCCTGGCCCGTCCGCGGCGAGAGCGCGAGCG
GAGCGAGCGATCGAGCGTCCCGTGGGCGGGTGCCGTAGGTGAGTTTACAC
ACCGCAGTCAAGGGGCAATTCGGGCTCGGGACTGGCCGGGCTATGGGCAA
GATTCTTAAAAAATTCCCCCGATCCCTCTGTCGCCAGGACATAAAAACAT
GCCGTGGAGACCGCCGGTGCATAGTGTCCAGGGGCGAGAGGATCAGTGGT
TCGCGAGCTTTTTTCACGGCCACGCTTCATTTTGCGGTTGCGGTGACGCT
GTTGGCCATCTTAATAGCATTGCTCCTCGCTTTCCTCGCGCCGGTCCACC
AAGGCCCCCTCCGGGGCTAGAGCAGCCTAACCCCCCGCAGCAGGGCCCGG
CCGGGCCCGGAGGGCCGCCCGCCATCTTGGCGCTGCCGGCTCCGCCCGCG
GAGCCTGACGACCCGCAGCCACGGCGTGGTGGTGGGGACGGTGGCGCCGC
CGCTGGCGCCGCAGGCGACCGTGGAGACCGAGACTACGACGAAGAAGAGC
TAGACGAGCTTTTCCGCGCCGCCGCCGAAGACGATTTGTAAGTAGGAGAT
GGCGCCGGCCTTACAGGCGCAGGAGGAGACGCGGGCGACGCAGACGCAGA
CGCAGACGCAGACATAAGCCCACCCTAGTACTCAGACAGTGGCAACCTGA
CGTTATCAGACACTGTAAGATAACAGGACGGATGCCCCTCATTATCTGTG
GAAAGGGGTCCACCCAGTTCAACTACATCACCCACGCGGACGACATCACC
CCCAGGGGAGCCTCCTACGGGGGCAACTTCACAAACATGACTTTCTCCCT
GGAGGCAATATACGAACAGTTTCTGTACCACAGAAACAGGTGGTCAGCCT
CCAACCACGACCTCGAACTCTGCAGATACAAGGGTACCACCCTAAAACTG
TACAGGCACCCAGATGTAGACTACATAGTCACCTACAGCAGAACGGGACC
CTTTGAGATCAGCCACATGACCTACCTCAGCACTCACCCCCTTCTCATGC
TGCTAAACAAACACCACATAGTGGTGCCCAGCCTAAAGACTAAGCCCAGG
GGCAGAAAGGCCATAAAAGTCAGAATAAGACCCCCCAAACTCATGAACAA
CAAGTGGTACTTCACCAGAGACTTCTGTAACATAGGCCTCTTCCAGCTCT
GGGCCACAGGCTTAGAACTCAGAAACCCCTGGCTCAGAATGAGCACCCTG
AGCCCCTGCATAGGCTTCAATGTCCTTAAAAACAGCATTTACACAAACCT
CAGCAACCTACCTCAGCACAGAGAAGACAGACTTAACATTATTAACAACA
CATTACACCCACATGACATAACAGGACCAAACAATAAAAAATGGCAGTAC
ACATATACCAAACTCATGGCCCCCATTTACTATTCAGCAAACAGGGCCAG
CACCTATGACTTACTACGAGAGTATGGCCTCTACAGTCCATACTACCTAA
ACCCCACAAGGATAAACCTTGACTGGATGACCCCCTACACACACGTCAGG
TACAATCCACTAGTAGACAAGGGCTTCGGAAACAGAATATACATACAGTG
GTGCTCAGAGGCAGATGTAAGCTACAACAGGACTAAATCCAAGTGTCTCT
TACAAGACATGCCCCTGTTTTTCATGTGCTATGGCTACATAGACTGGGCA
ATTAAAAACACAGGGGTCTCCTCACTAGCGAGAGACGCCAGAATCTGCAT
CAGGTGTCCCTACACAGAGCCACAGCTGGTGGGCTCCACAGAAGACATAG
GGTTCGTACCCATCACAGAGACCTTCATGAGGGGCGACATGCCGGTACTT
GCACCATACATACCGTTGAGCTGGTTTTGCAAGTGGTATCCCAACATAGC
TCACCAGAAGGAAGTACTTGAGGCAATCATTTCCTGCAGCCCCTTCATGC
CCCGTGACCAGGGCATGAACGGTTGGGATATTACAATAGGTTACAAAATG
GACTTCTTATGGGGCGGTTCCCCTCTCCCCTCACAGCCAATCGACGACCC
CTGCCAGCAGGGAACCCACCCGATTCCCGACCCCGATAAGCACCCTCGCC
TCCTACAAGTGTCGAACCCGAAACTGCTCGGACCGAGGACAGTGTTCCAC
AAGTGGGACATCAGACGTGGGCAGTTTAGCAAAAGAAGTATTAAAAGAGT
GTCAGAATACTCATCGGATGATGAATCTCTTGCGCCAGGTCTCCCATCAA
AGCGAAACAAGCTCGACTCGGCCTTCAGAGGAGAAAACCCAGAGCAAAAA
GAATGCTATTCTCTCCTCAAAGCACTCGAGGAAGAAGAGACCCCAGAAGA
AGAAGAACCAGCACCCCAAGAAAAAGCCCAGAAAGAGGAGCTACTCCACC
AGCTCCAGCTCCAGAGACGCCACCAGCGAGTCCTCAGACGAGGGCTCAAG
CTCGTCTTTACAGACATCCTCCGACTCCGCCAGGGAGTCCACTGGAACCC
CGAGCTCACATAGAGCCCCCACCTTACATACCAGACCTACTTTTTCCCAA
TACTGGTAAAAAAAAAAAATTCTCTCCCTTCGACTGGGAAACGGAGGCCC
AGCTAGCAGGGATATTCAAGCGTCCTATGCGCTTCTATCCCTCAGACACC
CCTCACTACCCGTGGTTACCCCCCAAGCGCGATATCCCGAAAATATGTAA
CATAAACTTCAAAATAAAGCTGCAAGAGTGAGTGATTCGAGGCCCTCCTC
TGTTCACTTAGCGGTGTCTACCTCTTAAAGTCACCAAGCACTCCGAGCGT
CAGCGAGGAGTGCGACCCTCCACCAAGGGGCAACTTCCTCGGGGTCCGGC
GCTACGCGCTTCGCGCTGCGCCGGACGCCTCGGACCCCCCCCCGACCCGA
ATCGCTCGCGCGATTCGGACCTGCGGCCTCGGGGGGGGTCGGGGGCTTTA
CTAAACAGACTCCGAGTTGCCACTGGACTCAGGAGCTGTGAATCAGTAAC
GAAAGTGAGTGGGGCCAGACTTCGCCATAGGGCCTTTAACTTGGGGTCGT
CTGTCGGTGGCTTCCGGGTCCGCCTGGGCGCCGCCATTTTAGCTTTAGAC
GCCATTTTAGGCCCTCGCGGGCACCCGTAGGCGCGTTTTAATGACGTCAC
GGCAGCCATTTTGTCGTGACGTTTGAGACACGTGATGGGGGCGTGCCTAA
ACCCGGAAGCATCCCTGGTCACGTGACTCTGACGTCACGGCGGCCATTTT
GTGCTGTCCGCCATCTTGTGACTTCCTTCCGCTTTTTCAAAAAAAAAGAG
GAAGTATGACAGTAGCGGCGGGGGGGCGGCCGCGTTCGCGCGCCGCCCAC
CAGGGGGTGCTGCGCGCCCCCCCCCGCGCATGCGCGGGGCCCCCCCCCGG
GGGGGCTCCGCCCCCCCGGCCCCCCCCCGTGCTAAACCCACCGCGCATGC
GCGACCACGCCCCCGCCGCC (SEQ ID NO: 1)
Annotations:

| Putative Domain | Base range |
|---|---|
| TATA Box | 84-90 |
| Cap Site | 107-114 |
| Transcriptional Start Site | 114 |
| 5′ UTR Conserved Domain | 177-247 |
| ORF2 | 299-691 |
| ORF2/2 | 299-687; 2137-2659 |
| ORF2/3 | 299-687; 2339-2831 |
| ORF2t/3 | 299-348; 2339-2831 |
| ORF1 | 571-2613 |
| ORF1/1 | 571-687; 2137-2613 |
| ORF1/2 | 571-687; 2339-2659 |
| Three open-reading frame region | 2325-2610 |
| Poly(A) Signal | 2813-2818 |
| GC-rich region | 3415-3570 |

TABLE A7
Exemplary Anellovirus amino acid sequences (Alphatorquevirus, Clade 1)
TTV-CT30F (Alphatorquevirus Clade 1)

| | |
|---|---|
| ORF2 | MPWRPPVHSVQGREDQWFASFFHGHASFCGCGDAVGHLNSIAPRFPRAGPPRPPPG |
| | LEQPNPPQQGPAGPGGPPAILALPAPPAEPDDPQPRRGGGDGGAAAGAAGDRGDRD |
| | YDEEELDELFRAAAEDDL (SEQ ID NO: 2) |
| ORF2/2 | MPWRPPVHSVQGREDQWFASFFHGHASFCGCGDAVGHLNSIAPRFPRAGPPRPPPG |
| | LEQPNPPQQGPAGPGGPPAILALPAPPAEPDDPQPRRGGGDGGAAAGAAGDRGDRD |
| | YDEEELDELFRAAAEDDFQSTTPASREPTRFPTPISTLASYKCRTRNCSDRGQCSTSG |
| | TSDVGSLAKEVLKECQNTHRMMNLLRQVSHQSETSSTRPSEEKTQSKKNAILSSKH |
| | SRKKRPQKKKNQHPKKKPRKRSYSTSSSSRDATSESSDEGSSSSLQTSSDSARESTGT |
| | PSSHRAPTLHTRPTFSQYW (SEQ ID NO: 3) |
| ORF2/3 | MPWRPPVHSVQGREDQWFASFFHGHASFCGCGDAVGHLNSIAPRFPRAGPPRPPPG |
| | LEQPNPPQQGPAGPGGPPAILALPAPPAEPDDPQPRRGGGDGGAAAGAAGDRGDRD |
| | YDEEELDELFRAAAEDDLSPIKAKQARLGLQRRKPRAKRMLFSPQSTRGRRDPRRR |
| | RTSTPRKSPERGATPPAPAPETPPASPQTRAQARLYRHPPTPPGSPLEPRAHIEPPPYIP |
| | DLLFPNTGKKKKFSPFDWETEAQLAGIFKRPMRFYPSDTPHYPWLPPKRDIPKICNIN |
| | FKIKLQE (SEQ ID NO: 4) |
| ORF2t/3 | MPWRPPVHSVQGREDQWSPIKAKQARLGLQRRKPRAKRMLFSPQSTRGRRDPRRR |
| | RTSTPRKSPERGATPPAPAPETPPASPQTRAQARLYRHPPTPPGSPLEPRAHIEPPPYIP |
| | DLLFPNTGKKKKFSPFDWETEAQLAGIFKRPMRFYPSDTPHYPWLPPKRDIPKICNIN |
| | FKIKLQE (SEQ ID NO: 5) |
| ORF1 | TAWWWGRWRRRWRRRRPWRPRLRRRRARRAFPRRRRRRFVSRRWRRPYRRRRR |
| | RGRRRRRRRRRHKPTLVLRQWQPDVIRHCKITGRMPLIICGKGSTQFNYITHADDIT |
| | PRGASYGGNFTNMTFSLEAIYEQFLYHRNRWSASNHDLELCRYKGTTLKLYRHPD |
| | VDYIVTYSRTGPFEISHMTYLSTHPLLMLLNKHHIVVPSLKTKPRGRKAIKVRIRPPK |
| | LMNNKWYFTRDFCNIGLFQLWATGLELRNPWLRMSTLSPCIGENVLKNSIYTNLSN |
| | LPQHREDRLNIINNTLHPHDITGPNNKKWQYTYTKLMAPIYYSANRASTYDLLREY |
| | GLYSPYYLNPTRINLDWMTPYTHVRYNPLVDKGFGNRIYIQWCSEADVSYNRTKSK |
| | CLLQDMPLFFMCYGYIDWAIKNTGVSSLARDARICIRCPYTEPQLVGSTEDIGFVPIT |
| | ETFMRGDMPVLAPYIPLSWFCKWYPNIAHQKEVLEAIISCSPFMPRDQGMNGWDITI |
| | GYKMDFLWGGSPLPSQPIDDPCQQGTHPIPDPDKHPRLLQVSNPKLLGPRTVFHKW |
| | DIRRGQFSKRSIKRVSEYSSDDESLAPGLPSKRNKLDSAFRGENPEQKECYSLLKALE |
| | EEETPEEEEPAPQEKAQKEELLHQLQLQRRHQRVLRRGLKLVFTDILRLRQGVHWN |
| | PELT (SEQ ID NO: 6) |
| ORF1/1 | TAWWWGRWRRRWRRRRPWRPRLRRRRARRAFPRRRRRRFPIDDPCQQGTHPIPDP |
| | DKHPRLLQVSNPKLLGPRTVFHKWDIRRGQFSKRSIKRVSEYSSDDESLAPGLPSKR |
| | NKLDSAFRGENPEQKECYSLLKALEEEETPEEEEPAPQEKAQKEELLHQLQLQRRH |
| | QRVLRRGLKLVFTDILRLRQGVHWNPELT (SEQ ID NO: 7) |
| ORF1/2 | TAWWWGRWRRRWRRRRPWRPRLRRRRARRAFPRRRRRRFVSHQSETSSTRPSEE |
| | KTQSKKNAILSSKHSRKKRPQKKKNQHPKKKPRKRSYSTSSSSRDATSESSDEGSSS |
| | SLQTSSDSARESTGTPSSHRAPTLHTRPTFSQYW (SEQ ID NO: 8) |

TABLE N8
Exemplary Anellovirus nucleic acid sequence (Alphatorquevirus, Clade 2)

| | |
|---|---|
| Name | TTV-P13-1 |
| Genus/Clade | Alphatorquevirus, Clade 2 |
| Accession Number | KT163896.1 |

Full Sequence: 3451 bp
10 20 30 40 50 60
| | | | | |
AATTTTGCTAAACAGACTCCGAGGTGCTCTTGGACACTGAGTGGGCGTAC
AGCAACGAAAGTGAGTGGGGCCAGACTTCGCCATAAGGCCTTTATCTTCG
GGTCTACATCATAATATAAAGATGTGCACTTCCGAATGGCTGAGTTTTTC
ACGCCATTCCGCAGCGGTGGAGCAGCGCAGCCACGACCCCCGCGTCCCGA
GGGCGGGTGCCGGAGGTGAGTTTACACACCGCAGTCAAGGGGCAATTCGG
GCTCGGGACTGGCCGGGCCCGGGCAAGGCTCTTAAAGCGAAACCATGTTC
CTCGGCAGGCCCTACCGCCACAGAAAGCGGCACCAGGCCGGCAAGAAAGG
GCCACTGCCACTGCCAAATCTGCAACCTGCACAGGAGAAACGGGCTGGTG
GTCCGTCCTTGATGGCCTCCGGACGCAGGGGATGGATGCCCCCGGACCTG
ACGGTCCAGGAGAGGGAGGATGCCTGGTGGACCAGCTTCTGCGCTAGCCA
CCGCAGCTTTTGTAGCTGCGACGATCCTGTGGGCCATATTAATACTCTCG
CCCGCGATAATAGTCCTCTGGCCCAGACTCCTACTACAACTTCAGGCCAG
GGGCCGCCGCCGCCGCCTACGCCTCCGCGGACGCCGGGGCCGCGCCCTGG
GTCTGCTCCGGACCAGGGGGGAAGGATCAGGGCCTCCTGGACCTACCCCC
TAGCCCCCGGAGGTCCCGGTAGCACGCCATGGCCTACTGGTGGGGCCGGA
GACGCCGGTGGCGCCGCTGGAGGAGGCGCCGGCGTCCTCTCCGCCGCCGC
CGGCGGTGGCGGAGAAGGCGACGCTGGCCCAGAAGGCGCCGGTGGAGGCG
AAGGAGACGACGTGCGAGACCTGCTCGCCGCTATCGAAGGAGACGTGGGC
GCAGACGGGTAAGGAGACGCCGTCGCCCCCAGAAACTAGTACTGACTCAG
TGGAATCCCCAGACTGTGAGAAAGTGTGTTATTAGGGGGTTTCTGCCCCT
GTTCTTCTGCGGACAGGGGGCCTACCACAGAAACTTTACAGACCACTATG
ACGATGTGTTCCCCAAGGGACCCAGCGGAGGTGGGCACGGGAGCATGGTG
TTCAACCTGTCCTTTCTGTACCAAGAGTTTAAGAAGCACCACAATAAGTG
GTCGCGCAGCAACCTGGACTTTGACTTAGTGAGATACAAGGGCACAGTGA
TAAAGCTGTACAGACACCAGGACTTTGACTACATAGTGTGGATAAGCAGG
ACCCCTCCCTTCCAGGAGAGCCTGCTCACAGTAATGACCCACCAGCCCAG
CGTCATGCTGCAGGCAAAAAAGTGCATAATAGTAAAGAGCTACAGGACCC
ACCCGGGGGGCAAACCCTATGTAACTGCAAAAGTTAGGCCCCCCAGACTC
CTAACTGACAAGTGGTACTTCCAGTCAGACTTCTGCAACGTTCCGCTTTT
TAGCCTACAGTTTGCCCTTGCGGAACTGCGGTTTCCGATCTGCTCACCAC
AAACTGACACCAATTGCATTAACTTCCTGGTGTTAGATGACATCTACTAC
AAGTTTCTAGATAATAAGCCTAAACAGAGTTCAGACCCTAATGACGAAAA
CAGAATAAAATTCTGGCACGGCCTATGGTCCACTATGAGATATTTAAACA
CCACCTACATAAACACACTGTTTCCAGGCACAGACAGTCTAGTGGCCGCC
AAAGATACTGACAATAGTGTAAATAAATACCCCAGCACAGCCACTAAACA
GCCCTACAAAGACAGTCAGTACATGCAAAATATATGGAATACATCAAAAA
TACATGCCTTATATACGTGGGTAGCAGAGACAAACTACAAAAGACTGCAG
GCCTACTACACACAGACCTACGGAGGCTACCAGAGACAATTTTTCACAGG
AAAACAGTACTGGGACTACAGAGTAGGCATGTTTAGTCCAGCCTTCCTGA
GTCCCAGCAGACTAAATCCCCAGAACCCAGGGGCATACACAGAGGTCTCC
TACAACCCCTGGACAGACGAGGGCACGGGCAACGTAGTGTGCCTGCAGTA
TCTGACTAAAGAGACCTCAGACTACAAACCAGGTGGTGGGAGCAAGTTCT
GCATAGAAGGTGTGCCTCTATGGGCAGCGCTGGTGGGATACGTAGACATG
TGTAAAAAAGAGGGCAAGGACCCGGGCATCAGACTAAACTGTCTCCTGTT
AGTCAAGTGTCCCTATACAAAGCCTCAGCTGTATGACAAAAAAAACCCCG
AGAAACTGTTTGTACCTTACTCCTATAACTTTGGGCACGGCAAGATGCCG
GGGGGAGACAAATACATACCCATAGAGTTCAAAGACAGGTGGTACCCCTG
CCTGCTCCACCAAGAGGAGTGGATAGAGGACATTGTCAGGTCGGGACCCT
TCGTTCCAAAAGACATGCCCAGCAGCGTCACCTGCATGATGAGGTACAGC
TCTCTTTTTAACTGGGGCGGTAATATAATCCAAGAACAGGCCGTGGAAGA
CCCCTGTAAGAAAGGCACCTTCGTCGTTCCCGGAACCAGTGGCATCGCTC
GCATACTACAAGTCAGCAACCCGGCCAAGCAGACCCCCACGACAACCTGG
CACTCGTGGGACTGGAGACGATCCCTCTTTACAGAGACGGGTCTTAAAAG
AATGCGCGAACAACAACCATATGATGAACTGTCTTATACGGGCCCTAAAA
AGCCAAAACTGTCCCTTCCCGCAGGGCCCGCCGTCCCCGGTGCCGCCGTC
GCCTCCTCCTGGTGGGAAACAAAACAGGTCACCTCGCCAGACGTCAGCGA
GACGGAGACCGAAGCAGAAGCCCACCAAGAGGAAGAGACGGAGCCGGAGG
AGGGAGTCCAGCTCCAGCAGCTGTGGGAGCAGCAACTCCTGCAAAAGCGA
CAGCTGGGAGTCGTGTTCCAGCAACTCCTCCGACTCAGACAGGGGGCGGA
GATCCACCCGGGCCTCGTATAATTCCTGGGCCCCAGAACCCGTACCTGCT
TTTCCCGGAGCAGGCCCCTCCAAAAGTGCCTATTTTTGACCCCTTTGGTC
AGAAAACAGAGCTAGAGCTGTGCGGCTGCTTCGACAGGCCGCCCAGGAAC
AACCCCTACGACCACCCCTTCTACCCCTGGCTGCCCAAAGAGCCTCCCTC
CTACTACCAGGGCTACAAAGTGTCTTTCAAACTAGGGTTCCACCCAGACA
AGCATGTGTGAACCCCGCCAATAAACCACTGCTGCTACACTGATTCTTAG
GCCGTGGGAGTCTCACTGGTCGGTGTCTACCTCTTAAGGTCACTAAGCAC
TCCGAGCGTTAGCGAGGAGTGCGACCCTACCCCCTGGGCCCACTTCTTCG
GAGCCGCGCGCTACGCCTTCGGCTGCGCGCGGCACCTCAGACCCCCGCTC
GTGCTGACACGCTTGCGCGTGTCAGACCACTTCGGGCTCGCGGGGGTCGG
G (SEQ ID NO: 9)
Annotations:

| Putative Domain | Base range |
|---|---|
| TATA Box | 112-119 |
| Initiator Element | 128-148 |
| Transcriptional Start Site | 148 |
| 5′ UTR Conserved Domain | 204-273 |
| ORF2 | 412-912 |
| ORF2/2 | 412-908; 2490-3039 |
| ORF2/3 | 412-908; 2725-3208 |
| ORF1 | 729-2972 |
| ORF1/1 | 729-908; 2490-2972 |
| ORF1/2 | 729-908; 2725-3039 |
| Three open-reading frame region | 2699-2969 |
| Poly(A) Signal | 3220-3225 |
| GC-rich region | 3302-3541 |

TABLE A8
Exemplary Anellovirus amino acid sequences (Alphatorquevirus, Clade 2)
TTV-P13-1 (Alphatorquevirus Clade 2)

| | |
|---|---|
| ORF2 | MASGRRGWMPPDLTVQEREDAWWTSFCASHRSFCSCDDPVGHINTLARDN |
| | SPLAQTPTTTSGQGPPPPPTPPRTPGPRPGSAPDQGGRIRASWTYPLAPGGPG |
| | STPWPTGGAGDAGGAAGGGAGVLSAAAGGGGEGDAGPEGAGGGEGDDV |
| | RDLLAAIEGDVGADG (SEQ ID NO: 10) |
| ORF2/2 | MASGRRGWMPPDLTVQEREDAWWTSFCASHRSFCSCDDPVGHINTLARDN |
| | SPLAQTPTTTSGQGPPPPPTPPRTPGPRPGSAPDQGGRIRASWTYPLAPGGPG |
| | STPWPTGGAGDAGGAAGGGAGVLSAAAGGGGEGDAGPEGAGGGEGDDV |
| | RDLLAAIEGDVGADGPWKTPVRKAPSSFPEPVASLAYYKSATRPSRPPRQPG |
| | TRGTGDDPSLQRRVLKECANNNHMMNCLIRALKSQNCPFPQGPPSPVPPSPP |
| | PGGKQNRSPRQTSARRRPKQKPTKRKRRSRRRESSSSSCGSSNSCKSDSWES |
| | CSSNSSDSDRGRRSTRASYNSWAPEPVPAFPGAGPSKSAYF (SEQ ID NO: 11) |
| ORF2/3 | MASGRRGWMPPDLTVQEREDAWWTSFCASHRSFCSCDDPVGHINTLARDN |
| | SPLAQTPTTTSGQGPPPPPTPPRTPGPRPGSAPDQGGRIRASWTYPLAPGGPG |
| | STPWPTGGAGDAGGAAGGGAGVLSAAAGGGGEGDAGPEGAGGGEGDDV |
| | RDLLAAIEGDVGADGARRPRCRRRLLLVGNKTGHLARRQRDGDRSRSPPRG |
| | RDGAGGGSPAPAAVGAATPAKATAGSRVPATPPTQTGGGDPPGPRIIPGPQN |
| | PYLLFPEQAPPKVPIFDPFGQKTELELCGCFDRPPRNNPYDHPFYPWLPKEPP |
| | SYYQGYKVSFKLGFHPDKHV (SEQ ID NO: 12) |
| ORF1 | MAYWWGRRRRWRRWRRRRRPLRRRRRWRRRRRWPRRRRWRRRRRRARP |
| | ARRYRRRRGRRRVRRRRRPQKLVLTQWNPQTVRKCVIRGFLPLFFCGQGAY |
| | HRNFTDHYDDVFPKGPSGGGHGSMVFNLSFLYQEFKKHHNKWSRSNLDFD |
| | LVRYKGTVIKLYRHQDFDYIVWISRTPPFQESLLTVMTHQPSVMLQAKKCII |
| | VKSYRTHPGGKPYVTAKVRPPRLLTDKWYFQSDFCNVPLFSLQFALAELRF |
| | PICSPQTDTNCINFLVLDDIYYKFLDNKPKQSSDPNDENRIKFWHGLWSTMR |
| | YLNTTYINTLFPGTDSLVAAKDTDNSVNKYPSTATKQPYKDSQYMQNIWNT |
| | SKIHALYTWVAETNYKRLQAYYTQTYGGYQRQFFTGKQYWDYRVGMFSP |
| | AFLSPSRLNPQNPGAYTEVSYNPWTDEGTGNVVCLQYLTKETSDYKPGGGS |
| | KFCIEGVPLWAALVGYVDMCKKEGKDPGIRLNCLLLVKCPYTKPQLYDKK |
| | NPEKLFVPYSYNFGHGKMPGGDKYIPIEFKDRWYPCLLHQEEWIEDIVRSGP |
| | FVPKDMPSSVTCMMRYSSLFNWGGNIIQEQAVEDPCKKGTFVVPGTSGIARI |
| | LQVSNPAKQTPTTTWHSWDWRRSLFTETGLKRMREQQPYDELSYTGPKKP |
| | KLSLPAGPAVPGAAVASSWWETKQVTSPDVSETETEAEAHQEEETEPEEGV |
| | QLQQLWEQQLLQKRQLGVVFQQLLRLRQGAEIHPGLV (SEQ ID NO: 13) |
| ORF1/1 | MAYWWGRRRRWRRWRRRRRPLRRRRRWRRRRRWPRRRRWRRRRRRARP |
| | ARRYRRRRGRRRAVEDPCKKGTFVVPGTSGIARILQVSNPAKQTPTTTWHS |
| | WDWRRSLFTETGLKRMREQQPYDELSYTGPKKPKLSLPAGPAVPGAAVASS |
| | WWETKQVTSPDVSETETEAEAHQEEETEPEEGVQLQQLWEQQLLQKRQLG |
| | VVFQQLLRLRQGAEIHPGLV (SEQ ID NO: 14) |
| ORF1/2 | MAYWWGRRRRWRRWRRRRRPLRRRRRWRRRRRWPRRRRWRRRRRRARP |
| | ARRYRRRRGRRRGPPSPVPPSPPPGGKQNRSPRQTSARRRPKQKPTKRKRRS |
| | RRRESSSSSCGSSNSCKSDSWESCSSNSSDSDRGRRSTRASYNSWAPEPVPAF |
| | PGAGPSKSAYF (SEQ ID NO: 15) |

TABLE N9
Exemplary Anellovirus nucleic acid sequence (Alphatorquevirus, Clade 3)

| | |
|---|---|
| Name | Ring1 |
| Genus/Clade | Alphatorquevirus, Clade 3 |
| Accession Number | AJ620231.1 |

Full Sequence: 3753 bp
1 10 20 30 40 50
| | | | | |
TGCTACGTCACTAACCCACGTGTCCTCTACAGGCCAATCGCAGTCTATGT
CGTGCACTTCCTGGGCATGGTCTACATAATTATATAAATGCTTGCACTTC
CGAATGGCTGAGTTTTTGCTGCCCGTCCGCGGAGAGGAGCCACGGCAGGG
GATCCGAACGTCCTGAGGGCGGGTGCCGGAGGTGAGTTTACACACCGAAG
TCAAGGGGCAATTCGGGCTCAGGACTGGCCGGGCTTTGGGCAAGGCTCTT
AAAAATGCACTTTTCTCGAATAAGCAGAAAGAAAAGGAAAGTGCTACTGC
TTTGCGTGCCAGCAGCTAAGAAAAAACCAACTGCTATGAGCTTCTGGAAA
CCTCCGGTACACAATGTCACGGGGATCCAACGCATGTGGTATGAGTCCTT
TCACCGTGGCCACGCTTCTTTTTGTGGTTGTGGGAATCCTATACTTCACA
TTACTGCACTTGCTGAAACATATGGCCATCCAACAGGCCCGAGACCTTCT
GGGCCACCGGGAGTAGACCCCAACCCCCACATCCGTAGAGCCAGGCCTGC
CCCGGCCGCTCCGGAGCCCTCACAGGTTGATTCGAGACCAGCCCTGACAT
GGCATGGGGATGGTGGAAGCGACGGAGGCGCTGGTGGTTCCGGAAGCGGT
GGACCCGTGGCAGACTTCGCAGACGATGGCCTCGATCAGCTCGTCGCCGC
CCTAGACGACGAAGAGTAAGGAGGCGCAGACGGTGGAGGAGGGGGAGACG
AAAAACAAGGACTTACAGACGCAGGAGACGCTTTAGACGCAGGGGACGAA
AAGCAAAACTTATAATAAAACTGTGGCAACCTGCAGTAATTAAAAGATGC
AGAATAAAGGGATACATACCACTGATTATAAGTGGGAACGGTACCTTTGC
CACAAACTTTACCAGTCACATAAATGACAGAATAATGAAAGGCCCCTTCG
GGGGAGGACACAGCACTATGAGGTTCAGCCTCTACATTTTGTTTGAGGAG
CACCTCAGACACATGAACTTCTGGACCAGAAGCAACGATAACCTAGAGCT
AACCAGATACTTGGGGGCTTCAGTAAAAATATACAGGCACCCAGACCAAG
ACTTTATAGTAATATACAACAGAAGAACCCCTCTAGGAGGCAACATCTAC
ACAGCACCCTCTCTACACCCAGGCAATGCCATTTTAGCAAAACACAAAAT
ATTAGTACCAAGTTTACAGACAAGACCAAAGGGTAGAAAAGCAATTAGAC
TAAGAATAGCACCCCCCACACTCTTTACAGACAAGTGGTACTTTCAAAAG
GACATAGCCGACCTCACCCTTTTCAACATCATGGCAGTTGAGGCTGACTT
GCGGTTTCCGTTCTGCTCACCACAAACTGACAACACTTGCATCAGCTTCC
AGGTCCTTAGTTCCGTTTACAACAACTACCTCAGTATTAATACCTTTAAT
AATGACAACTCAGACTCAAAGTTAAAAGAATTTTTAAATAAAGCATTTCC
AACAACAGGCACAAAAGGAACAAGTTTAAATGCACTAAATACATTTAGAA
CAGAAGGATGCATAAGTCACCCACAACTAAAAAAACCAAACCCACAAATA
AACAAACCATTAGAGTCACAATACTTTGCACCTTTAGATGCCCTCTGGGG
AGACCCCATATACTATAATGATCTAAATGAAAACAAAAGTTTGAACGATA
TCATTGAGAAAATACTAATAAAAAACATGATTACATACCATGCAAAACTA
AGAGAATTTCCAAATTCATACCAAGGAAACAAGGCCTTTTGCCACCTAAC
AGGCATATACAGCCCACCATACCTAAACCAAGGCAGAATATCTCCAGAAA
TATTTGGACTGTACACAGAAATAATTTACAACCCTTACACAGACAAAGGA
ACTGGAAACAAAGTATGGATGGACCCACTAACTAAAGAGAACAACATATA
TAAAGAAGGACAGAGCAAATGCCTACTGACTGACATGCCCCTATGGACTT
TACTTTTTGGATATACAGACTGGTGTAAAAAGGACACTAATAACTGGGAC
TTACCACTAAACTACAGACTAGTACTAATATGCCCTTATACCTTTCCAAA
ATTGTACAATGAAAAAGTAAAAGACTATGGGTACATCCCGTACTCCTACA
AATTCGGAGCGGGTCAGATGCCAGACGGCAGCAACTACATACCCTTTCAG
TTTAGAGCAAAGTGGTACCCCACAGTACTACACCAGCAACAGGTAATGGA
GGACATAAGCAGGAGCGGGCCCTTTGCACCTAAGGTAGAAAAACCAAGCA
CTCAGCTGGTAATGAAGTACTGTTTTAACTTTAACTGGGGCGGTAACCCT
ATCATTGAACAGATTGTTAAAGACCCCAGCTTCCAGCCCACCTATGAAAT
ACCCGGTACCGGTAACATCCCTAGAAGAATACAAGTCATCGACCCGCGGG
TCCTGGGACCGCACTACTCGTTCCGGTCATGGGACATGCGCAGACACACA
TTTAGCAGAGCAAGTATTAAGAGAGTGTCAGAACAACAAGAAACTTCTGA
CCTTGTATTCTCAGGCCCAAAAAAGCCTCGGGTCGACATCCCAAAACAAG
AAACCCAAGAAGAAAGCTCACATTCACTCCAAAGAGAATCGAGACCGTGG
GAGACCGAGGAAGAAAGCGAGACAGAAGCCCTCTCGCAAGAGAGCCAAGA
GGTCCCCTTCCAACAGCAGTTGCAGCAGCAGTACCAAGAGCAGCTCAAGC
TCAGACAGGGAATCAAAGTCCTCTTCGAGCAGCTCATAAGGACCCAACAA
GGGGTCCATGTAAACCCATGCCTACGGTAGGTCCCAGGCAGTGGCTGTTT
CCAGAGAGAAAGCCAGCCCCAGCTCCTAGCAGTGGAGACTGGGCCATGGA
GTTTCTCGCAGCAAAAATATTTGATAGGCCAGTTAGAAGCAACCTTAAAG
ATACCCCTTACTACCCATATGTTAAAAACCAATACAATGTCTACTTTGAC
CTTAAATTTGAATAAACAGCAGCTTCAAACTTGCAAGGCCGTGGGAGTTT
CACTGGTCGGTGTCTACCTCTAAAGGTCACTAAGCACTCCGAGCGTAAGC
GAGGAGTGCGACCCTCCCCCCTGGAACAACTTCTTCGGAGTCCGGCGCTA
CGCCTTCGGCTGCGCCGGACACCTCAGACCCCCCCTCCACCCGAAACGCT
TGCGCGTTTCGGACCTTCGGCGTCGGGGGGGTCGGGAGCTTTATTAAACG
GACTCCGAAGTGCTCTTGGACACTGAGGGGGTGAACAGCAACGAAAGTGA
GTGGGGCCAGACTTCGCCATAAGGCCTTTATCTTCTTGCCATTTGTCAGT
GTCCGGGGTCGCCATAGGCTTCGGGCTCGTTTTTAGGCCTTCCGGACTAC
AAAAATCGCCATTTTGGTGACGTCACGGCCGCCATCTTAAGTAGTTGAGG
CGGACGGTGGCGTGAGTTCAAAGGTCACCATCAGCCACACCTACTCAAAA
TGGTGGACAATTTCTTCCGGGTCAAAGGTTACAGCCGCCATGTTAAAACA
CGTGACGTATGACGTCACGGCCGCCATTTTGTGACACAAGATGGCCGACT
TCCTTCCTCTTTTTCAAAAAAAAGCGGAAGTGCCGCCGCGGCGGCGGGGG
GCGGCGCGCTGCGCGCGCCGCCCAGTAGGGGGAGCCATGCGCCCCCCCCC
GCGCATGCGCGGGGCCCCCCCCCGCGGGGGGCTCCGCCCCCCGGCCCCCC
CCG (SEQ ID NO: 16)
Annotations:

| Putative Domain | Base range |
|---|---|
| TATA Box | 83-88 |
| Cap Site | 104-111 |
| Transcriptional Start Site | 111 |
| 5′ UTR Conserved Domain | 170-240 |
| ORF2 | 336-719 |
| ORF2/2 | 336-715; 2363-2789 |
| ORF2/3 | 336-715; 2565-3015 |
| ORF2t/3 | 336-388; 2565-3015 |
| ORF1 | 599-2830 |
| ORF1/1 | 599-715; 2363-2830 |
| ORF1/2 | 599-715; 2565-2789 |
| Three open-reading frame region | 2551-2786 |
| Poly(A) Signal | 3011-3016 |
| GC-rich region | 3632-3753 |

TABLE A9
Exemplary Anellovirus amino acid sequences (Alphatorquevirus, Clade 3)
Ring1 (Alphatorquevirus Clade 3)

| | |
|---|---|
| ORF2 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEE (SEQ ID NO: 17) |
| ORF2/2 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEELLKTPASSPPMKYPVPVTSLEEYKSSTRGSWDRTTRSGHGT |
| | CADTHLAEQVLRECQNNKKLLTLYSQAQKSLGSTSQNKKPKKKAHIHSKENRDRG |
| | RPRKKARQKPSRKRAKRSPSNSSCSSSTKSSSSSDRESKSSSSSS (SEQ ID NO: 18) |
| ORF2/3 | MSFWKPPVHNVTGIQRMWYESFHRGHASFCGCGNPILHITALAETYGHPTGPRPSG |
| | PPGVDPNPHIRRARPAPAAPEPSQVDSRPALTWHGDGGSDGGAGGSGSGGPVADFA |
| | DDGLDQLVAALDDEEPKKASGRHPKTRNPRRKLTFTPKRIETVGDRGRKRDRSPLA |
| | REPRGPLPTAVAAAVPRAAQAQTGNQSPLRAAHKDPTRGPCKPMPTVGPRQWLFP |
| | ERKPAPAPSSGDWAMEFLAAKIFDRPVRSNLKDTPYYPYVKNQYNVYFDLKFE |
| | (SEQ ID NO: 19) |
| ORF2t/3 | MSFWKPPVHNVTGIQRMWPKKASGRHPKTRNPRRKLTFTPKRIETVGDRGRKRDR |
| | SPLAREPRGPLPTAVAAAVPRAAQAQTGNQSPLRAAHKDPTRGPCKPMPTVGPRQ |
| | WLFPERKPAPAPSSGDWAMEFLAAKIFDRPVRSNLKDTPYYPYVKNQYNVYFDLK |
| | FE (SEQ ID NO: 20) |
| ORF1 | MAWGWWKRRRRWWFRKRWTRGRLRRRWPRSARRRPRRRRVRRRRRWRRGRRK |
| | TRTYRRRRRFRRRGRKAKLIIKLWQPAVIKRCRIKGYIPLIISGNGTFATNFTSHINDR |
| | IMKGPFGGGHSTMRFSLYILFEEHLRHMNFWTRSNDNLELTRYLGASVKIYRHPDQ |
| | DFIVIYNRRTPLGGNIYTAPSLHPGNAILAKHKILVPSLQTRPKGRKAIRLRIAPPTLFT |
| | DKWYFQKDIADLTLFNIMAVEADLRFPFCSPQTDNTCISFQVLSSVYNNYLSINTEN |
| | NDNSDSKLKEFLNKAFPTTGTKGTSLNALNTFRTEGCISHPQLKKPNPQINKPLESQ |
| | YFAPLDALWGDPIYYNDLNENKSLNDIIEKILIKNMITYHAKLREFPNSYQGNKAFC |
| | HLTGIYSPPYLNQGRISPEIFGLYTEIIYNPYTDKGTGNKVWMDPLTKENNIYKEGQS |
| | KCLLTDMPLWTLLFGYTDWCKKDTNNWDLPLNYRLVLICPYTFPKLYNEKVKDY |
| | GYIPYSYKFGAGQMPDGSNYIPFQFRAKWYPTVLHQQQVMEDISRSGPFAPKVEKP |
| | STQLVMKYCFNFNWGGNPIIEQIVKDPSFQPTYEIPGTGNIPRRIQVIDPRVLGPHYSF |
| | RSWDMRRHTFSRASIKRVSEQQETSDLVFSGPKKPRVDIPKQETQEESSHSLQRESR |
| | PWETEEESETEALSQESQEVPFQQQLQQQYQEQLKLRQGIKVLFEQLIRTQQGVHV |
| | NPCLR (SEQ ID NO: 21) |
| ORF1/1 | MAWGWWKRRRRWWFRKRWTRGRLRRRWPRSARRRPRRRRIVKDPSFQPTYEIPG |
| | TGNIPRRIQVIDPRVLGPHYSFRSWDMRRHTFSRASIKRVSEQQETSDLVFSGPKKPR |
| | VDIPKQETQEESSHSLQRESRPWETEEESETEALSQESQEVPFQQQLQQQYQEQLKL |
| | RQGIKVLFEQLIRTQQGVHVNPCLR (SEQ ID NO: 22) |
| ORF1/2 | MAWGWWKRRRRWWFRKRWTRGRLRRRWPRSARRRPRRRRAQKSLGSTSQNKK |
| | PKKKAHIHSKENRDRGRPRKKARQKPSRKRAKRSPSNSSCSSSTKSSSSSDRESKSSS |
| | SSS (SEQ ID NO: 23) |

TABLE N10
Exemplary Anellovirus nucleic acid sequence (Alphatorquevirus, Clade 4)

| | |
|---|---|
| Name | TTV-HD20a |
| Genus/Clade | Alphatorquevirus, Clade 4 |
| Accession Number | FR751492.1 |

Full Sequence 3878 bp
1 10 20 30 40 50
| | | | | |
AAATACGTCACTAACCACGTGACTCCCACAGGCCAACCACAGTCTATGTC
GTGCACTTCCTGGGCATGGTCTACGTGATAATATAAAGCGGTGCACTTCC
GAATGGCTGAGTTTTCCACGCCCGTCCGCAGCGAGATCGCGACGTAGGAG
CGATCGAGCGTCCCGAGGGCGGGTGCCGGAGGTGAGTTTACACACCGCAG
TCAAGGGGCAATTCGGGCTCGGGAGGCCGGGCCATGGGCAAGGCTCTTAA
AAAGCTATGTTTCTCGGTAAAATCTACAGGAAGAAAAGGAAACTGCTTCT
GCAGGCTGTGCGTGCTCCGCAGACGCCATCTTCCATGAGCCGCTGCTGGT
GTCCCCCTCGGGGTGATGTCTCCTCCCGCGAGTCTCGATGGTACGAGGCG
GTTCGAGGAAGCCACGATGCTTTTTGTGGCTGTAGTGATCCTATTCTTCA
TCTTTCTCGTCTGGCTGCACGTTTTAACCATCAGGGACCTCCGACGCCCC
CCACGGACGACCGTGCGCCGCAGAATACCCCAGTGAGACGCCTGCTGCCT
CTCCCCAGCTACCCCGGCGAGGGTCCCCAGGCTAGATGGCCTGGTGGGGA
TGGAGGCGCCGCTGGTGGCGACCGAAGAGAAGGTGGAGATGGCGGCGCGC
GCGCCGCCGAAGACGAGTACCAGCCCGAAGACCTAGACGAGCTTTTCGGC
GCTATCGAACAAGAACAGTAAGGAGGAGGCGAAGGGGGAGGCGGAGGGGC
TACCGGCGCCGTTACAGACTGAGACGCTATGCCAGACGCAGGTTCCGACG
CAAAAAGATAGTACTGACTCAGTGGAACCCCCAGACTACCAGAAAATGTA
TAATAAGGGGCATGATGCCAGTACTGTGGGCCGGCATGGGTACGGGGGGC
AGAAACTATGCAGTGAGGTCAGATGACTATGTGGTGAACAAAGGGTTCGG
GGGCTCCTTCGCCACGGAGACCTTCTCCCTGAAGGTTCTCTATGACCAGT
TTCAAAGGGGCTTCAACAGGTGGTCCCACACTAACGAGGACCTAGACCTG
GCCCGCTACAGGGGCTGCAGGTGGACTTTTTACAGACATAAAGACACAGA
CTTTATAGTGTACTTTACAAACAATCCTCCCATGAAGACCAACCAGTTCT
CCGCGCCCCTGACGACCCCCGGCATGCTCATGCGCAGTAAATACAAAGTC
CTCATTCCCAGCTTCCAGACCAGACCCAAGGGTCGCAAAACAGTAACCGT
TAAAATAAGACCCCCCAAACTATTTCAAGACAAGTGGTACACCCAGCAGG
ACCTGTGTTCAGTTCCTCTTGTCCAACTGAACGTGACCGCAGCTGATTTC
ACACATCCGTTCGGCTCACCACTAACTGAAACTCCTTGCGTAGAGTTCCA
GGTGCTGGGTGACTTGTACAATACATGTCTCAATATCGACCTTCCGCAAT
TTAGTGAATTAGGAGAAATAACTAGTGCCTACTCAAAACCAAACTCAAAT
AACCTAAAAGAATTATACAAAGAATTGTTCACAAAAGCCACATCAGGACA
CTACTGGCAGACATTCATAACCAACAGCATGGTCAGAGCACACATAGATG
CAGACAAAGCTAAAGAAGCACAAAGAGCATCCACCACACCCTCATACAAC
AATGACCCCTTCCCCACAATACCTGTTAAATCAGAGTTTGCACAGTGGAA
AAAGAAATTCACAGACACTAGAGACAGCCCCTTTCTTTTTGCCACTTACC
ATCCCGAAGCTATAAAAGACACAATTATGAAAATGAGAGAGAACAACTTT
AAGCTAGAGACAGGACCCAATGACAAGTATGGAGACTACACAGCACAGTA
CCAAGGAAACACACACATGCTAGACTACTACCTTGGCTTTTACAGCCCCA
TATTCCTCTCAGATGGAAGGTCTAACGTAGAATTCTTCACTGCCTACAGA
GACATAGTATACAATCCCTTCTTAGACAAGGCCCAGGGCAACATGGTGTG
GTTTCAGTACCACACAAAGACAGACAACAAGTTTAAAAAACCAGAGTGCC
ACTGGGAAATCAAAGACATGCCCCTGTGGGCCCTCCTAAACGGATATGTA
GACTACTTAGAGACTCAAATACAGTATGGTGACCTCAGTAAAGAAGGGAA
AGTCCTCATCAGGTGTCCCTACACCAAGCCAGCACTAGTAGACCCCAGAG
ACGACACTGCAGGATATGTAGTCTACAACAGAAACTTTGGCAGAGGCAAG
TGGATAGACGGAGGGGGCTACATCCCCCTGCACGAGAGGACAAAATGGTA
CGTGATGCTCAGATACCAGACGGACGTCTTCCATGACATAGTGACCTGTG
GGCCCTGGCAGTACAGAGACGACAACAAAAACAGCCAGCTAGTGGCCAAA
TACCGCTTCAGCTTTATATGGGGAGGTAACACTGTCCACTCTCAGGTCAT
CAGAAACCCGTGCAAAGACAACCAAGTATCCGGTCCCCGTCGACAGCCTA
GGGATATACAAGTCGTTGACCCGCAACGCATCACGCCGCCGTGGGTCCTC
CACAGCTTCGACCAGCGAAGAGGCCTCTTTACTGAAACAGCTCTCAGGCG
CCTGCTCCAGGAACCACTACCTGGCGAGTATGCTGTTAGCACCCTCAGGA
CACCCCTCCTCTTTCTACCCTCAGAATACCAGCGAGAAGACGGCGCTGCA
GAAAGCGCCTCAGGTTCACCGGCCAAAAGACCCCGTATCTGGTCAGAAGA
GAGTCAGACGGAGACGATCTCCTCGGAGGAGAACCCGGCGGAGACGACGA
GGGAGCTCCTCCAGCGAAAGCTCCGAGAGCAGCGAGCACTCCAGTTCCAA
CTCCAGCACTTCGCGGTCCAACTCGCCAAGACCCAGGCGAATCTCCACGT
AAACCCCCTGTTATCTTTCCCGCAATGAATAAGGTCTTTCTGTTTCCCCC
AGAGGGTCCCAAGCCCATCCTGGGCAAAGAGGCCTGGCAGGACGAGTACG
AGACCTGCAGGGTCTGGAACAGACCTGCCAGAACCCACCACACAGACACC
CCCTTCTATCCCTGGGCCCCCCACAAGTTCCATGTAAGCTTCAAACTTGG
CTTCCAATAAAATTACTAGGCCGTGGAACTCTCACTGGTCGGTGTCTACC
TCTTAAGGTCACTAAGCACTCCGAGCGTCAGCGAGGAGTGCGACCCTCTA
CCCTGGTGCAACGCCCTCGGCGGCCGCGCGCTACGCCTTCGGCTGCGCGC
GGCACCTCGGACCCCCGCTCGTGCTGACGCGCTCGCGCGCGTCAGACCAC
TTCGGGCTCGCGGGGGTCGGGAATTTTGCTAAACAGACTCCGAGTTGCCA
TTGGACACTGTAGCTGTGAATCAGTAACGAAAGTGAGTGGGGCCAGACTT
CGCCATAGGGCCTTTATCTTCTTGCCATTGGTCCGTGTAGGGGGTCGCCA
TAGGCTTCGACCTCCCTTTTAGGCCTTCCGGACTACAAAAATGGCGGATT
CAGTGACGTCACGGCCGCCATTTTAAGTAGGTGCCGTCCAGGACTGCAGT
TCCGGGTCAGAGTGCATCCTCGGCGGAACCTGCACAAAATGGCGGTCAAT
ATCTTCCGGGTCAAAGGTCACACCTACGTCATAAGTCACGTGACTGGGTC
CTGCTACGTCATATGCGGAAGTAGGCCCCGCCACGTGACTCGTCACGTGG
GCGCTGCGTCACGGCGGCCATTTTGTATCACAAAATGGCGGACTTCCTTC
CTCTTTTTTAAAAATAACGGCCCAGCGGCGGCGCGCGCGCTTCGCGCGCG
CGCCGGGGGGCTCCGCCCCCCCCCGCGCATGCGCGGGGCCCCCCCCCGCG
GGGGGCTCCGCCCCCCGGTCCCCCCCCG (SEQ ID NO: 24)
Annotations:

| Putative Domain | Base range |
|---|---|
| TATA Box | 82-87 |
| Initiator Element | 95-115 |
| Transcriptional Start Site | 115 |
| 5′ UTR Conserved Domain | 170-238 |
| ORF2 | 335-721 |
| ORF2/2 | 335-717; 2446-2902 |
| ORF2/3 | 335-717; 2675-3109 |
| ORF1 | 586-2928 |
| ORF1/1 | 586-717; 2446-2928 |
| ORF1/2 | 586-717; 2675-2902 |
| Three open-reading frame region | 2640-2899 |
| Poly(A) Signal | 3106-3114 |
| GC-rich region | 3768-3878 |

TABLE A10
Exemplary Anellovirus amino acid sequences (Alphatorquevirus, Clade 4)
TTV-HD20a (Alphatorquevirus Clade 4)

| | |
|---|---|
| ORF2 | MSRCWCPPRGDVSSRESRWYEAVRGSHDAFCGCSDPILHLSRLAARFNHQG |
| | PPTPPTDDRAPQNTPVRRLLPLPSYPGEGPQARWPGGDGGAAGGDRREGGD |
| | GGARAAEDEYQPEDLDELFGAIEQEQ (SEQ ID NO: 25) |
| ORF2/2 | MSRCWCPPRGDVSSRESRWYEAVRGSHDAFCGCSDPILHLSRLAARFNHQG |
| | PPTPPTDDRAPQNTPVRRLLPLPSYPGEGPQARWPGGDGGAAGGDRREGGD |
| | GGARAAEDEYQPEDLDELFGAIEQEQSSETRAKTTKYPVPVDSLGIYKSLTR |
| | NASRRRGSSTASTSEEASLLKQLSGACSRNHYLASMLLAPSGHPSSFYPQNT |
| | SEKTALQKAPQVHRPKDPVSGQKRVRRRRSPRRRTRRRRRGSSSSESSESSE |
| | HSSSNSSTSRSNSPRPRRIST (SEQ ID NO: 26) |
| ORF2/3 | MSRCWCPPRGDVSSRESRWYEAVRGSHDAFCGCSDPILHLSRLAARFNHQG |
| | PPTPPTDDRAPQNTPVRRLLPLPSYPGEGPQARWPGGDGGAAGGDRREGGD |
| | GGARAAEDEYQPEDLDELFGAIEQEQIPARRRRCRKRLRFTGQKTPYLVRRE |
| | SDGDDLLGGEPGGDDEGAPPAKAPRAASTPVPTPALRGPTRQDPGESPRKPP |
| | VIFPAMNKVFLFPPEGPKPILGKEAWQDEYETCRVWNRPARTHHTDTPFYP |
| | WAPHKFHVSFKLGFQ (SEQ ID NO: 27) |
| ORF1 | MAWWGWRRRWWRPKRRWRWRRARRRRRVPARRPRRAFRRYRTRTVRR |
| | RRRGRRRGYRRRYRLRRYARRRFRRKKIVLTQWNPQTTRKCIIRGMMPVL |
| | WAGMGTGGRNYAVRSDDYVVNKGFGGSFATETFSLKVLYDQFQRGFNRW |
| | SHTNEDLDLARYRGCRWTFYRHKDTDFIVYFTNNPPMKTNQFSAPLTTPGM |
| | LMRSKYKVLIPSFQTRPKGRKTVTVKIRPPKLFQDKWYTQQDLCSVPLVQL |
| | NVTAADFTHPFGSPLTETPCVEFQVLGDLYNTCLNIDLPQFSELGEITSAYSK |
| | PNSNNLKELYKELFTKATSGHYWQTFITNSMVRAHIDADKAKEAQRASTTP |
| | SYNNDPFPTIPVKSEFAQWKKKFTDTRDSPFLFATYHPEAIKDTIMKMRENN |
| | FKLETGPNDKYGDYTAQYQGNTHMLDYYLGFYSPIFLSDGRSNVEFFTAYR |
| | DIVYNPFLDKAQGNMVWFQYHTKTDNKFKKPECHWEIKDMPLWALLNGY |
| | VDYLETQIQYGDLSKEGKVLIRCPYTKPALVDPRDDTAGYVVYNRNFGRGK |
| | WIDGGGYIPLHERTKWYVMLRYQTDVFHDIVTCGPWQYRDDNKNSQLVA |
| | KYRFSFIWGGNTVHSQVIRNPCKDNQVSGPRRQPRDIQVVDPQRITPPWVLH |
| | SFDQRRGLFTETALRRLLQEPLPGEYAVSTLRTPLLFLPSEYQREDGAAESAS |
| | GSPAKRPRIWSEESQTETISSEENPAETTRELLQRKLREQRALQFQLQHFAVQ |
| | LAKTQANLHVNPLLSFPQ (SEQ ID NO: 28) |
| ORF1/1 | MAWWGWRRRWWRPKRRWRWRRARRRRRVPARRPRRAFRRYRTRTVIRN |
| | PCKDNQVSGPRRQPRDIQVVDPQRITPPWVLHSFDQRRGLFTETALRRLLQE |
| | PLPGEYAVSTLRTPLLFLPSEYQREDGAAESASGSPAKRPRIWSEESQTETISS |
| | EENPAETTRELLQRKLREQRALQFQLQHFAVQLAKTQANLHVNPLLSFPQ |
| | (SEQ ID NO: 29) |
| ORF1/2 | MAWWGWRRRWWRPKRRWRWRRARRRRRVPARRPRRAFRRYRTRTNTSE |
| | KTALQKAPQVHRPKDPVSGQKRVRRRRSPRRRTRRRRRGSSSSESSESSEHS |
| | SSNSSTSRSNSPRPRRIST (SEQ ID NO: 30) |

TABLE N11
Exemplary Anellovirus nucleic acid sequence (Alphatorquevirus, Clade 5)

| | |
|---|---|
| Name | TTV-16 (TUS01) |
| Genus/Clade | Alphatorquevirus, Clade 5 |
| Accession Number | AB017613.1 |

full Sequence: 3818 bp
1 10 20 30 40 50
| | | | | |
AAGTCCGCCACTAACCACGTGACTCCCGCAGGCCAACCCAGTACTATGTC
GTCCACTTCCTGGGACGAGTCTACGTCCTGATATAAGTAAGTGCACTTCC
GAATGGCTGAGTTTTCCACGCCCGTCCGCAGCGAGAACGCCACGGAGGGG
AGTCCGCGCGTCCCGAGGGCGGGTGCCGGAGGTGAGTTTACACACCGCAG
TCAAGGGGCAATTCGGGCTCGGGACTGGCCGGGCCCCGGGCAAGGCTCTT
AAAAAATGCACTTTCGCAGAGTGCGAGCGAAAAGGAAACTGCTACTGCAA
GCTGTGCGAGCTCCACCGAAGGCACCTGCCATGAGCTTCACCACACCTAC
TATTAATGCCGGGATCCGAGAGCAGCAATGGTTCGAGTCCACCCTTAGAT
CCCACCACTCGTTCTGTGGCTGTGGTGATCCCGTGCTTCATTTTACTAAC
CTTGCTACTCGCTTTAACTATCTGCCTGCTACCTCTTCGCCTCTGGACCC
TCCCGGCCCAGCGCCGCGAGGCCGCCCGGCGCTCCGCCGCCTCCCGGCAC
TCCCTTCAGCCCCCGCGACCCCTTCTAGAGAACTAGCATGGCCTACTGGT
TCAGAAGGTGGGGCTGGAGGCCGAGGCGCCGGTGGAGAAGGTGGCGCCGC
CGTCGAAGGAGACTACCGAGAAGAAGAACTAGACGAGCTGTTCGCGGCCT
TGGAAGAAGACGCAAACCAAGGGTAAGGAGGCGCCGCAGAACTCGCAGAC
GTACCTACAGACGGGGGTGGAGACGCAGGAGGTACATAAGACGGGGGCGA
CGCAAAAAGAAACTCATACTGACTCAGTGGAACCCGGCAATAGTTAAGAG
GTGCAACATTAAGGGCGGACTTCCAATAATTATATGCGGAGAGCCCAGGG
CAGCCTTTAACTATGGCTACCACATGGAGGACTACACTCCTCAACCTTTC
CCCTTCGGAGGGGGAATGAGCACAGTGACTTTCTCTCTGAAAGCCTTGTA
TGACCAGTACCTAAAACACCAAAACAGGTGGACTTTCTCAAACGACCAGC
TAGACCTCGCCAGATACAGGGGCTGTAAACTAAGGTTCTACAGAAGCCCC
GTCTGTGACTTTATAGTACACTACAACCTAATACCTCCACTAAAAATGAA
CCAGTTCACAAGTCCCAACACGCACCCGGGACTACTCATGCTCAGCAAAC
ACAAGATAATAATTCCCAGCTTTCAAACAAGACCTGGGGGCAGACGCTTT
GTTAAAATAAGACTTAATCCCCCCAAACTATTTGAAGACAAGTGGTACAC
TCAGCAAGACCTGTGCAAGGTTCCGCTCGTTAGTATTACAGCAACTGCGG
CTGACTTGCGGTATCCGTTCTGCTCACCACAAACGAACAACCCTTGCACC
ACCTTCCAGGTACTGCGCAAGAACTACAATACAGTTATAGGAACTTCCGT
AAAAGACCAAGAGTCCACACAAGACTTTGAAAATTGGCTTTATAAAACAG
ACTCACACTATCAAACATTTGCCACAGAGGCTCAACTAGGCAGAATTCCT
GCATTTAATCCTGATGGCACTAAAAACACTAAACAGCAGTCGTGGCAAGA
TAACTGGAGCAAAAAAAATTCACCATGGACAGGTAACTCAGGTACATACC
CACAAACAACCAGTGAAATGTACAAAATTCCATATGACAGTAACTTCGGC
TTTCCCACATACAGAGCCCAAAAAGACTACATTTTAGAAAGAAGACAGTG
CAACTTTAACTATGAAGTTAATAATCCAGTTAGCAAAAAAGTATGGCCAC
AACCTAGTACAACAACACCCACAGTAGACTACTATGAATACCACTGTGGA
TGGTTCAGCAACATATTCATAGGCCCCAACAGATACAACCTACAGTTTCA
AACAGCATATGTAGACACCACATACAACCCACTAATGGACAAGGGCAAAG
GCAACAAAATATGGTTTCAATATCTGTCTAAAAAGGGCACAGACTACAAT
GAAAAACAATGCTACTGCACCCTAGAAGACATGCCCCTATGGGCAATATG
CTTTGGATACACTGACTATGTAGAGACTCAACTAGGACCCAATGTGGACC
ATGAAACAGCAGGCTTAATAATTATGATCTGTCCATACACTCAACCACCT
ATGTATGACAAAAACAGACCTAACTGGGGATACGTAGTCTATGACACAAA
CTTTGGCAATGGAAAAATGCCCTCAGGAAGTGGCCAAGTCCCAGTATACT
GGCAATGCCGATGGAGGCCCATGCTGTGGTTCCAACAACAAGTACTCAAT
GACATCTCAAAGACTGGACCGTACGCCTACAGAGACGAATATAAAAATGT
ACAACTGACTCTCTACTACAACTTTATTTTTAACTGGGGGGGCGACATGT
ATTACCCACAGGTCGTTAAAAACCCCTGTGGAGACTCCGGAATCGTTCCC
GGTTCCGGTAGATTCACTCGAGAAGTACAAGTCGTTAGCCCGCTTTCCAT
GGGACCGGCCTACATCTTCCACTACTTCGACTCCAGACGCGGGTTCTTTA
GTGAAAAAGCTCTTAAAAGAATGCAACAACAACAAGAATTTGATGAATCT
TTTACATTCAAACCTAAGAGACCCAAACTTTCTACAGCAGCCGCAGAAAT
CCTCCAGCTCGAAGAAGACTCGACTTCAGGGGAAGGAAAATCGCCACTAC
AGCAAGAAGAGAAAGAAGTCGAAGTCCTCCAAACGCCGACAGTACAGCTC
CAGCTCCAGCGAAACATCCAGGAGCAGCTCGCAATCAAGCAGCAGCTCCA
ATTCCTCTTGCTCCAACTCCTCAAAACCCAATCCAATTTGCATTTAAACC
CACAATTTTTAAGCCCTTCATAAAATATGACATGTTTGGGGACCCCCTTC
CTCACCCCCCAACAGCCGAAGAGTGGGAAACAGAGTACCAGTGCTGTAAG
GCCTTTAACAGACCACCTAGAACCAACCTAAAAGACACCCCCTTCTACCC
CTGGGTACCTAAACCTAAACCTCAATTCCGTGTATCTTTTAAACTTGGTT
TTCAATAAACAAGGCCGTGGGAGTTTCACTTGTCGGTGTCAACCTCTTAA
GGTCACTAAGCACTCCGAGCGTAAGCGAGGAGTGCGACCCTCCCCCCTGG
GGCAACTCCCTCGAAGTCCGGCGCTACGCGCTTCGCGCTGCGCCGGACAT
CTCGGACCCCCCCTCCACCCGAAACGCTTGCGCGTTTCGGACCTTCGGCG
TCGGGGGGGTCGGGGGCTTTACTAAACAGACTCCGAGGTGCCATTGGACA
CTGAGGGGATGAACAGCAACGAAAGTGAGTGGGGCCAGACTTCGCCATAA
GGCCTTTATCTTCTTGCCATTTGTCAGTATAGAGGGTCGCCATAGGCTTC
GGCCTCCATTTTAACCTCTAAAAACTACCAAAATGGCCGTTCCAGTGACG
TCACAGCCGCCATTTTAAGTAGCTGACGTCAAGGATTGACGTGAAGGTTA
AAGGTCATCCTCGGCGGAAGCTACACAAAATGGTGGACAACATCTTCCGG
GTCAAAGGTCGTGCACACGTCATAAGTCACGTGGTGGGGACCCGCTGTAA
CCCGGAAGTAGGCCCCGTCACGTGATTTGTCACGTGTGTACACGTCACAA
CCGCCATTTTGTTTTACAAAATGGCTGACTTCCTTCCTCTTTTTTAAAAA
AAACGGCCGTGCGGCGGCGCGCGCGCTTCGCGCGCGCGCCGGGGGCTGCC
GCCCCCCCCCGCGCATGCGCGCGGGGCCCCCCCCCGCGGGGGGCTCCGCC
CCCCGGCCCCCCCCCCCG (SEQ ID NO: 31)
Annotations:

| Putative Domain | Base range |
|---|---|
| TATA Box | 82-86 |
| Initiator Element | 100-115 |
| Transcriptional Start Site | 115 |
| 5′ UTR Conserved Domain | 170-240 |
| ORF2 | 331-726 |
| ORF2/2 | 331-722; 2412-2847 |
| ORF2/3 | 331-722; 2638-3058 |
| ORF2t/3 | 331-380; 2638-3058 |
| ORF1 | 588-2873 |
| ORF1/1 | 588-722; 2412-2873 |
| ORF1/2 | 588-722; 2638-2847 |
| Three open-reading frame region | 2699-2969 |
| Poly(A) Signal | 3220-3225 |
| GC-rich region | 3302-3541 |

TABLE A11
Exemplary Anellovirus amino acid sequences (Alphatorquevirus, Clade 5)
TTV-16-TUS01 (Alphatorquevirus Clade 5)

| | |
|---|---|
| ORF2 | MSFTTPTINAGIREQQWFESTLRSHHSFCGCGDPVLHFTNLATRFNYLPATSS |
| | PLDPPGPAPRGRPALRRLPALPSAPATPSRELAWPTGSEGGAGGRGAGGEGG |
| | AAVEGDYREEELDELFAALEEDANQG (SEQ ID NO: 32) |
| ORF2/2 | MSFTTPTINAGIREQQWFESTLRSHHSFCGCGDPVLHFTNLATRENYLPATSS |
| | PLDPPGPAPRGRPALRRLPALPSAPATPSRELAWPTGSEGGAGGRGAGGEGG |
| | AAVEGDYREEELDELFAALEEDANQGSLKTPVETPESFPVPVDSLEKYKSLA |
| | RFPWDRPTSSTTSTPDAGSLVKKLLKECNNNKNLMNLLHSNLRDPNFLQQP |
| | QKSSSSKKTRLQGKENRHYSKKRKKSKSSKRRQYSSSSSETSRSSSQSSSSSN |
| | SSCSNSSKPNPICI (SEQ ID NO: 33) |
| ORF2/3 | MSFTTPTINAGIREQQWFESTLRSHHSFCGCGDPVLHFTNLATRENYLPATSS |
| | PLDPPGPAPRGRPALRRLPALPSAPATPSRELAWPTGSEGGAGGRGAGGEGG |
| | AAVEGDYREEELDELFAALEEDANQGSRRNPPARRRLDFRGRKIATTARRE |
| | RSRSPPNADSTAPAPAKHPGAARNQAAAPIPLAPTPQNPIQFAFKPTIFKPFIK |
| | YDMFGDPLPHPPTAEEWETEYQCCKAFNRPPRTNLKDTPFYPWVPKPKPQF |
| | RVSFKLGFQ (SEQ ID NO: 34) |
| ORF2t/3 | MSFTTPTINAGIREQQCSRRNPPARRRLDFRGRKIATTARRERSRSPPNADST |
| | APAPAKHPGAARNQAAAPIPLAPTPQNPIQFAFKPTIFKPFIKYDMFGDPLPH |
| | PPTAEEWETEYQCCKAFNRPPRTNLKDTPFYPWVPKPKPQFRVSFKLGFQ |
| | (SEQ ID NO: 35) |
| ORF1 | MAYWFRRWGWRPRRRWRRWRRRRRRLPRRRTRRAVRGLGRRRKPRVRR |
| | RRRTRRRTYRRGWRRRRYIRRGRRKKKLILTQWNPAIVKRCNIKGGLPIIICG |
| | EPRAAFNYGYHMEDYTPQPFPFGGGMSTVTFSLKALYDQYLKHQNRWTFS |
| | NDQLDLARYRGCKLRFYRSPVCDFIVHYNLIPPLKMNQFTSPNTHPGLLMLS |
| | KHKIIIPSFQTRPGGRRFVKIRLNPPKLFEDKWYTQQDLCKVPLVSITATAAD |
| | LRYPFCSPQTNNPCTTFQVLRKNYNTVIGTSVKDQESTQDFENWLYKTDSH |
| | YQTFATEAQLGRIPAFNPDGTKNTKQQSWQDNWSKKNSPWTGNSGTYPQT |
| | TSEMYKIPYDSNFGFPTYRAQKDYILERRQCNFNYEVNNPVSKKVWPQPST |
| | TTPTVDYYEYHCGWFSNIFIGPNRYNLQFQTAYVDTTYNPLMDKGKGNKIW |
| | FQYLSKKGTDYNEKQCYCTLEDMPLWAICFGYTDYVETQLGPNVDHETAG |
| | LIIMICPYTQPPMYDKNRPNWGYVVYDTNFGNGKMPSGSGQVPVYWQCR |
| | WRPMLWFQQQVLNDISKTGPYAYRDEYKNVQLTLYYNFIFNWGGDMYYP |
| | QVVKNPCGDSGIVPGSGRFTREVQVVSPLSMGPAYIFHYFDSRRGFFSEKAL |
| | KRMQQQQEFDESFTFKPKRPKLSTAAAEILQLEEDSTSGEGKSPLQQEEKEV |
| | EVLQTPTVQLQLQRNIQEQLAIKQQLQFLLLQLLKTQSNLHLNPQFLSPS |
| | (SEQ ID NO: 36) |
| ORF1/1 | MAYWFRRWGWRPRRRWRRWRRRRRRLPRRRTRRAVRGLGRRRKPRVVK |
| | NPCGDSGIVPGSGRFTREVQVVSPLSMGPAYIFHYFDSRRGFFSEKALKRMQ |
| | QQQEFDESFTFKPKRPKLSTAAAEILQLEEDSTSGEGKSPLQQEEKEVEVLQT |
| | PTVQLQLQRNIQEQLAIKQQLQFLLLQLLKTQSNLHLNPQFLSPS (SEQ ID |
| | NO: 37) |
| ORF1/2 | MAYWFRRWGWRPRRRWRRWRRRRRRLPRRRTRRAVRGLGRRRKPRQPQ |
| | KSSSSKKTRLQGKENRHYSKKRKKSKSSKRRQYSSSSSETSRSSSQSSSSSNS |
| | SCSNSSKPNPICI (SEQ ID NO: 38) |

TABLE N12
Exemplary Anellovirus nucleic acid sequence (Alphatorquevirus, Clade 6)

| | |
|---|---|
| Name | TTV-TJN02 |
| Genus/Clade | Alphatorquevirus, Clade 6 |
| Accession Number | AB028669.1 |

Full Sequence: 3794 bp
1 10 20 30 40 50
| | | | | |
CCCGAAGTCCGTCACTAACCACGTGACTCCTGTCGCCCAATCAGAGTGTA
TGTCGTGCATTTCCTGGGCATGGTCTACATCCTGATATAACTAAGTGCAC
TTCCGAATGGCTGAGTTTTCCACGCCCGTCCGCAGCGAGGGAGCGACGGA
GGAGCTCCCGAGCGTCCCGAGGGCGGGTGCCGGAGGTGAGTTTACACACC
GCAGTCAAGGGGCAATTCGGGCTCGGGACTGGCCGGGCTATGGGCAAGGC
TCTTAGGGTCTTCATTCTTAATATGTTTCTTGGCAGAGTTTACCGCCACA
AGAAAAGGAAAGTGCTACTGTCCACACTGCGAGCTCCACAGGCGTCTCGC
AGGGCTATGAGTTGGCGACCCCCGGTACACGATGCACCCGGCATCGAGCG
CAATTGGTACGAGGCCTGTTTCAGAGCCCACGCTGGAGCTTGTGGCTGTG
GCAATTTTATTATGCACCTTAATCTTTTGGCTGGGCGTTATGGTTTTACT
CCGGGGTCAGCGCCGCCAGGTGGTCCTCCTCCGGGCACCCCGCAGATAAG
GAGAGCCAGGCCTAGTCCCGCCGCACCAGAGCAGCCCGCTGCCCTACCAT
GGCATGGGGATGGTGGAGATGGCGGCGCCGCTGGCCCGCCAGACGCTGGA
GGAGACGCCGTCGCCGGCGCCCCGTACGGAGAACAAGAGCTCGCCGACCT
GCTCGACGCTATAGAAGACGACGAACAGTAAGAACCAGGCGAAGGCGGTG
GGGGCGCAGACGGTACAGACGGGGCTGGAGACGCAGGACTTATGTGAGAA
AGGGGCGACACAGAAAAAAGAAAAAGAGACTGATACTGAGACAGTGGCAA
CCAGCCACAAGACGCAGATGTACCATAACTGGGTACCTGCCCATAGTGTT
CTGCGGCCACACTAGGGGCAATAAAAACTATGCACTACACTCTGACGACT
ACACCCCCCAAGGACAACCATTIGGAGGGGCTCTAAGCACTACCTCATTC
TCTTTAAAAGTACTATTTGACCAGCATCAGAGAGGACTAAACAAGTGGTC
TTTTCCAAACGACCAACTAGACCTCGCCAGATATAGAGGCTGCAAATTTA
TATTTTATAGAACAAAACAAACTGACTGGGTGGGCCAGTATGACATATCA
GAACCCTACAAGCTAGACAAATACAGCTGCCCCAACTATCACCCTGGAAA
CATGATTAAGGCAAAGCACAAATTTTTAATACCAAGCTATGACACTAATC
CTAGAGGCAGACAAAAAATTATAGTTAAAATTCCCCCCCCAGACCTCTTT
GTAGACAAGTGGTACACTCAAGAGGATCTGTGTTCCGTTAATCTTGTGTC
ACTTGCGGTTTCTGCGGCTTCCTTTCTCCACCCATTCGGCTCACCACAAA
CTGACAACCCTTGCTACACCTTCCAGGTGTTGAAAGAGTTCTACTATCAG
GCAATAGGCTTCTCTGCAAGCACACAAGCAATGACATCAGTATTAGACAC
GCTATACACACAAAACAGTTATTGGGAATCTAATCTAACTCAGTTTTATG
TACTTAATGCAAAAAAAGGCAGTGATACAACACAGCCTTTAACTAGCAAT
ATGCCAACTCGTGAAGAGTTTATGGCAAAAAAAAATACCAATTACAACTG
GTATACATACAAGGCCGCGTCAGTAAAAAATAAACTACATCAAATGAGAC
AAACCTATTTTGAGGAGTTAACCTCTAAGGGGCCACAAACAACAAAAAGT
GAGGAAGGCTACAGTCAGCACTGGACCACCCCCTCCACAAACGCCTACGA
ATATCACTTAGGAATGTTTAGTGCAATATTTCTAGCCCCAGACAGGCCAG
TACCTAGATTTCCATGCGCCTACCAAGATGTAACTTACAACCCCTTAATG
GACAAAGGGGTGGGAAACCACATTTGGTTTCAGTACAACACAAAGGCAGA
CACTCAGCTAATAGTCACAGGAGGGTCCTGCAAAGCACACATACAAGACA
TACCACTGTGGGCGGCCTTCTATGGATACAGTGACTTTATAGAGTCAGAA
CTAGGCCCCTTTGTAGATGCAGAGACGGTAGGCTTAGTGTGTGTAATATG
CCCTTATACAAAACCCCCCATGTACAACAAGACAAACCCCGCCATGGGCT
ACGTGTTCTATGACAGAAACTTTGGTGACGGAAAATGGACTGACGGACGG
GGCAAAATAGAGCCCTACTGGCAAGTTAGGTGGAGGCCCGAAATGCTTTT
CCAAGAAACTGTAATGGCAGACCTAGTTCAGACTGGGCCCTTTAGCTACA
AAGACGAACTTAAAAACAGCACCCTAGTGTGCAAGTACAAATTCTATTTC
ACCTGGGGAGGTAACATGATGTTCCAACAGACGATCAAAAACCCGTGCAA
GACGGACGGACAACCCACCGACTCCAGTAGACACCCTAGAGGAATACAAG
TGGCGGACCCGGAACAAATGGGACCCCGCTGGGTGTTCCACTCCTTTGAC
TGGCGAAGGGGCTATCTTAGCGAGAAAGCTCTCAAACGCCTGCAAGAAAA
ACCTCTTGACTATGACGAATATTTTACACAACCAAAAAGACCTAGAATCT
TTCCTCCAACAGAATCAGCAGAGGGAGAGTTCCGAGAGCCCGAAAAAGGC
TCGTATTCAGAGGAAGAAAGGTCGCAAGCCTCTGCCGAAGAGCAGACGCA
GGAGGCGACAGTACTCCTCCTCAAGCGACGACTCAGAGAGCAACAGCAGC
TCCAGCAGCAGCTCCAATTCCTCACCCGAGAAATGTTCAAAACGCAAGCG
GGTCTCCACCTAAACCCTATGTTATTAAACCAGCGATAAACCAAGTGTAC
CTGTTTCCAGAGAGGGCCCCAAAACCCCCTCCTAGCAGCCAAGACTGGCA
GCAGGAGTACGAGGCCTGCGCAGCCTGGGACAGGCCCCCTAGATACAATC
TGTCCTCTCCTCCTTTCTACCCCAGCTGCCCTTCAAAATTCTGTGTAAAA
TTCAGCCTTGGCTTTAAATAAATGGCAACTTTACTGTGCAAGGCCGTGGG
AGTTTCACTGGTCGGTGTCTACCTCTAAAGGTCACTAAGCACTCCGAGCG
TTAGCGAGGAGTGCGACCCTTCCCCCTGACTCAACTTCTTCGGAGCCGCG
CGCTACGCCTTCGGCTGCGCGCGGCACCTCAGACCCCCGCTCGTGCTGAC
ACGCTCGCGCGTGTCAGACCACTTCGGGCTCGCGGGGGTCGGGAATTTTG
CTAAACAGACTCCGAGTTGCTCTTGGACACTGAGGGGGCATATCAGTAAC
GAAAGTGAGTGGGGCCAGACTTCGCCATAAGGCCTTTATCTTCTTGCCAT
TGGATAGTATCGAGGGTTGCCATAGGCTTCGACCTCCATTTTAGGCCTTC
CGGACTACAAAAATGGCCGTTTTAGTGACGTCACGGCCGCCATTTTAAGT
AAGGCGGAAGCAGCTCGGCGTACACAAAATGGCGGCGGAGCACTTCCGGC
TTGCCCAAAATGGTGGGCAACTTCTTCCGGGTCAAAGGTCACAGCTACGT
CACAAGTCACGTGGGGAGGGTTGGCGTTTAACCCGGAAGCCAATCCTCTT
ACGTGGCCTGTCACGTGACTTGTACGTCACGACCACCATTTTGTTTTACA
AAATGGCCGACTTCCTTCCTCTTTTTTAAAAATAACGGTTCGGCGGCGGC
GCGCGCGCTACGCGCGCGCGCCGGGGGGCTGCCGCCCCCCCCCCGCGCAT
GCGCGGGGCCCCCCCCCGCGGGGGGCTCCGCCCCCCGGCCCCCC (SEQ ID NO: 39)
Annotations:

| Putative Domain | Base range |
|---|---|
| TATA Box | 89-90 |
| Cap Site | 107-114 |
| Transcriptional Start Site | 114 |
| 5′ UTR Conserved Domain | 174-244 |
| ORF2 | 357-731 |
| ORF2/2 | 357-727; 2381-2813 |
| ORF2/3 | 357-727; 2619-3021 |
| ORF2t/3 | 357-406; 2619-3021 |
| ORF1 | 599-2839 |
| ORF1/1 | 599-727; 2381-2839 |
| ORF1/2 | 599-727; 2619-2813 |
| Three open-reading frame region | 2596-2810 |
| Poly(A) Signal | 3017-3022 |
| GC-rich region | 3691-3794 |

TABLE A12
Exemplary Anellovirus amino acid sequences (Alphatorquevirus, Clade 6)
TTV-TJN02 (Alphatorquevirus Clade 6)

| | |
|---|---|
| ORF2 | MSWRPPVHDAPGIERNWYEACFRAHAGACGCGNFIMHLNLLAGRYGFTPGSAPPG |
| | GPPPGTPQIRRARPSPAAPEQPAALPWHGDGGDGGAAGPPDAGGDAVAGAPYGEQ |
| | ELADLLDAIEDDEQ (SEQ ID NO: 40) |
| ORF2/2 | MSWRPPVHDAPGIERNWYEACFRAHAGACGCGNFIMHLNLLAGRYGFTPGSAPPG |
| | GPPPGTPQIRRARPSPAAPEQPAALPWHGDGGDGGAAGPPDAGGDAVAGAPYGEQ |
| | ELADLLDAIEDDEQRSKTRARRTDNPPTPVDTLEEYKWRTRNKWDPAGCSTPLTGE |
| | GAILARKLSNACKKNLLTMTNILHNQKDLESFLQQNQQRESSESPKKARIQRKKGR |
| | KPLPKSRRRRRQYSSSSDDSESNSSSSSSSNSSPEKCSKRKRVST (SEQ ID NO: 41) |
| ORF2/3 | MSWRPPVHDAPGIERNWYEACFRAHAGACGCGNFIMHLNLLAGRYGFTPGSAPPG |
| | GPPPGTPQIRRARPSPAAPEQPAALPWHGDGGDGGAAGPPDAGGDAVAGAPYGEQ |
| | ELADLLDAIEDDEHRGRVPRARKRLVFRGRKVASLCRRADAGGDSTPPQATTQRAT |
| | AAPAAAPIPHPRNVQNASGSPPKPYVIKPAINQVYLFPERAPKPPPSSQDWQQEYEA |
| | CAAWDRPPRYNLSSPPFYPSCPSKFCVKFSLGFK (SEQ ID NO: 42) |
| ORF2t/3 | MSWRPPVHDAPGIERNCRGRVPRARKRLVFRGRKVASLCRRADAGGDSTPPQATT |
| | QRATAAPAAAPIPHPRNVQNASGSPPKPYVIKPAINQVYLFPERAPKPPPSSQDWQQ |
| | EYEACAAWDRPPRYNLSSPPFYPSCPSKFCVKFSLGFK (SEQ ID NO: 43) |
| ORF1 | MAWGWWRWRRRWPARRWRRRRRRRPVRRTRARRPARRYRRRRTVRTRRRRWG |
| | RRRYRRGWRRRTYVRKGRHRKKKKRLILRQWQPATRRRCTITGYLPIVFCGHTRG |
| | NKNYALHSDDYTPQGQPFGGALSTTSFSLKVLFDQHQRGLNKWSFPNDQLDLARY |
| | RGCKFIFYRTKQTDWVGQYDISEPYKLDKYSCPNYHPGNMIKAKHKFLIPSYDTNP |
| | RGRQKIIVKIPPPDLFVDKWYTQEDLCSVNLVSLAVSAASFLHPFGSPQTDNPCYTF |
| | QVLKEFYYQAIGFSASTQAMTSVLDTLYTQNSYWESNLTQFYVLNAKKGSDTTQPL |
| | TSNMPTREEFMAKKNTNYNWYTYKAASVKNKLHQMRQTYFEELTSKGPQTTKSE |
| | EGYSQHWTTPSTNAYEYHLGMFSAIFLAPDRPVPRFPCAYQDVTYNPLMDKGVGN |
| | HIWFQYNTKADTQLIVTGGSCKAHIQDIPLWAAFYGYSDFIESELGPFVDAETVGLV |
| | CVICPYTKPPMYNKTNPAMGYVFYDRNFGDGKWTDGRGKIEPYWQVRWRPEMLF |
| | QETVMADLVQTGPFSYKDELKNSTLVCKYKFYFTWGGNMMFQQTIKNPCKTDGQ |
| | PTDSSRHPRGIQVADPEQMGPRWVFHSFDWRRGYLSEKALKRLQEKPLDYDEYFT |
| | QPKRPRIFPPTESAEGEFREPEKGSYSEEERSQASAEEQTQEATVLLLKRRLREQQQL |
| | QQQLQFLTREMFKTQAGLHLNPMLLNQR (SEQ ID NO: 44) |
| ORF1/1 | MAWGWWRWRRRWPARRWRRRRRRRPVRRTRARRPARRYRRRRTTIKNPCKTDG |
| | QPTDSSRHPRGIQVADPEQMGPRWVFHSFDWRRGYLSEKALKRLQEKPLDYDEYF |
| | TQPKRPRIFPPTESAEGEFREPEKGSYSEEERSQASAEEQTQEATVLLLKRRLREQQQ |
| | LQQQLQFLTREMFKTQAGLHLNPMLLNQR (SEQ ID NO: 45) |
| ORF1/2 | MAWGWWRWRRRWPARRWRRRRRRRPVRRTRARRPARRYRRRRTQRESSESPKK |
| | ARIQRKKGRKPLPKSRRRRRQYSSSSDDSESNSSSSSSSNSSPEKCSKRKRVST (SEQ |
| | ID NO: 46) |

TABLE N13
Exemplary Anellovirus nucleic acid sequence (Alphatorquevirus, Clade 7)

| | |
|---|---|
| Name | TTV-HD16d |
| Genus/Clade | Alphatorquevirus, Clade 7 |
| Accession Number | FR751479.1 |

Full Sequence: 3866 bp
1 10 20 30 40 50
| | | | | |
AAGTCCGTCACTAACCACGTGACTCCCGCAGGCCAATCAGAGTCTATGTC
GTGCACTTCCTGGGCATGGTCTACGTTCTCATATAACTAACTGCACTTCC
GAATGGCTGAGTTTTCCACGCCCGTCCGCAGCGGCAGCACCACGGAGGGT
GATCCCCGCGTCCCGAGGGCGGGTGCCGAAGGTGAGTTTACACACCGCAG
TCAAGGGGCAATTCGGGCTCGGGACTGGCCGGGCTATGGGCAAGGCTCTT
AGGGCTTTCATTGTTAAAAATGTTTCTCGGCAGGCCTTACAGGAGAAAGA
AAAGGGCGCTGTCACTGCCTGGCGTGCGAGCTGCACAGGCGAAACAACCT
GGTGATATGAGCTGGAGCCGTCCAGTACATAATGCCGCCGGGATCGAAAG
GCAGTGGTTCGAATCCACCTTTAGATCCCACGCTAGTTGCTGTGGCTGCG
GCAATTTTGTTAATCATATTAATGTACTGGCTGCTCGCTACGGCTTTACT
GGGGGGCCGACGCCGCCAGGTGGTCCTGGGCCGCGTCCACAACTGAGGCC
CGCGCTTCCCGCGCCGGACCCCGACCCCCAGGCGCCCAACCGTGAGCCAT
GGCGTGGAGCTGGTGGTGGCAACGATGGAGAAGGCGCCGCTGGAAACCCA
GGAGGCGCCGCTGGAGACGTCTACGATGGAGAAGACCTAGACGCGCTGTT
CGCCGCCGTCGTCGAGGACGTAGAGTAAGGAGGCGGAGGTGGGCGCGTAG
ACGGGGGCGACGCAGACGGTACGCCACCAGACGAAAGAGACGTTATAGGG
GTCGCCGCTTTAAAAAGAAACTAGTACTGACTCAGTGGCACCCTAATACC
ATGAGACGCTGCTTAATCAAGGGCATAGTCCCCCTGGTAATATGCGGCCA
CACCAGGTGGAACTACAACTACGCCCTCCATAGCAAGGACTACACAGAGG
AGGGTCGCTACCCTCACGGGGGGGCCCTCAGCACCACTACGTGGTCCCTT
AAGGTGCTGTATGACGAGCACCTCAAACACCACGACTTCTGGGGCTATCC
CAACAACCAGCTAGACCTGGCCAGGTACAAGGGGGCCAAGTTCACCTTCT
ACAGACACAAAAAGACTGACTTTATAATATTCTTTAACAGAAAGCCTCCC
TTTAAGCTAAACAAGTACAGCTGTGCCTCCTATCACCCAGGCATGCTGAT
GCAGCAGAGACACAAGATCCTGCTACCCAGCTACGAAACTAAACCCAAGG
GCAGGCCAAAGATAACAGTTAGAATAAAGCCCCCCACTCTGTTAGAGGAC
AAGTGGTACACCCAGCAGGACCTGTGCGACGTTAACCTGTTGCAACTTGT
GGTCACTGCGGCTGACTTTCGACATCCACTCTGCTCACCACAAACGAACA
CTCCAACCACAACCTTCCAGGTGTTGAAAGACATCTATTATGACACTATG
AGCATATCTGAACCCACAGACTCCTACACTAGTGTTAACAATAAAAGTAC
AACACAAACTTTTACTAACTACTCAAACACCTTAGAAAACATTCTGTACA
CACGAGCCTCCTACTGGAACTCGTTCCACGCCACTGAATACCTAAACCCC
AACATCATATACAAAAACGGTGAAAAACTATTCAAAGAACATGAAGACTT
AATAACCTGGATGACCCAAACTAACAATACCGGGTTTCTAACTAAAAACA
ACACAGCTTTTGGCAACAACAGCTACAGGCCCAATGCAGACAAAATTAAA
AAAGCCAGAAAGACATACTGGAACGCCCTAATAGGCACCAACGACCTGGC
CACTAATATAGGCCAGGCCAGAGCAGAAAGGTTCGAGTACCACCTAGGCT
GGTACTCCCCCATATTTCTCAGCAGACACAGGAGCAACATGAACTTTGCC
AGGGCCTACCAAGACGTCACATACAACCCCAACTGTGACAGGGGAGTTAA
CAACAGGGTGTGGGTTCAGCCTCTAACTAAACCCACCACAGAGTTCGACG
AGAAAAGGTGTAAGTGCGTAGTGCAGCACCTGCCTCTGTGGGCGGCTCTG
TACTGCTACCAAGACTTTGTAGAGGAGGAGCTGGGGTCCTCCTCAGAGAT
ATTAAATTCATGCCTACTGGTATTACAGTGCCCTTACACCTTTCCCCCAA
TGTATGACAAAAAGCTACCAGACAAGGGATTCGTGTTTTATGACTCCCTT
TTTGGAGACGGCAAAATGTCTGACGGACGCGGACAGGTGGACATTTTCTG
GCAACAGCGATGGTACCCTCGCTTAGCCACTCAGATGCAAGTCATGCACG
ACATCACCATGACGGGCCCCTTCTCCTACCGAGACGAGCTAGTTAGCACC
CAACTGACTGCCAAGTACACCTTTGACTTTATGTGGGGCGGAAATATGAT
CTCCACACAGATCATCAAGAACCCCTGCAAAGACAGTGGACTGGAACCCG
CCTACCCCGGTAGACAGCGTCGCGACTTACAAATTGTTGACCCATACTCC
ATGGGCCCCCAATTCTCGTTCCACAACTGGGACTACAGACATGGCCTTTT
TGGCCAAGACGCTATCGACAGAGTGTCTAAACAACCAAAAGATGATGCAG
ACTATCCTAACCCATACAAAAGGCCTAGATATTTTCCACCCACAGACCAA
GCCGCCCAAGAGCAAGAAAAAGACTTCAGTTTCCTCAAAACAGCACCGTC
GAACTCAGAAGAGAGCGATCAAGAAGTCCTCCAAGAAACGCAAGTACTCC
GATTCCAGCCAGAGCAGCACAAGCAACTCCACCTGCAGCTCGCAGAGCGG
CAGCGAATCGGAGAGCAACTCCGATACCTACTCCAACAGATGTTCAAAAC
TCAGGCCAATCTCCACCTAAACCCATATACATTTACCCAGCTGTAAAGCA
GGTGTTTATGTTTGACCCCCCGGGCCCTAAGGCTATCTCGGGCGCCAAGG
CCTGGGAGGACGAGTTCCTCACCGCAAAAGTGTGGAACCGCCCGGTACGC
AAGTACTACTCAGACACCCCCTACTACCCCTGGGCCCCCAAACCCCAGTA
CTCTGTCAGTTTCAAACTCGGCTGGAAATAAAAAAAGCCTGCTCCACTGT
ACTAGGCCGTGGGAGTTTCACTCGTCGGTGTCTACCTCTTAAGGTCACCA
AGCACTCCGAGCGTCAGCGAGGAGTGCGACCCTTGGGGTGGGGTGCAACG
CCCTCGGCGGCCGCGCGCTACGCCTTCGGCTGCGCGCGGCACCTCGGACC
CCCGCTCGTGCTGACGCGCTTGCGCGCGTCAGACCACTTCGGGCTCGCGG
GGGTCGGAAATTTTGCTAAACAGACTCCGAGTTGCCATTGGACACTGGAG
CCGTGAATCAGTAACGAAAGTGAGTGGGGCCAGACTTCGCCATAAGGCCT
TTATCTTTTTGCCATTTGTCCGTGGGGAAGGGTCGCTGCAAGCGCGGACC
CCGTTTTCACCCCTTCCGGACTACAAAAATAGCGCATTAGTGACGTCACG
GCCGCCATTTTAAGTAAGGCGGAAGCAACTCCACTTTCTCACAAAATGGC
GGCGGAGCACTTCCGGCTTGCCCAAAATGGCCGCCAAAAACATCCGGGTC
AAAGTTCGCCGCTACGTCATAAGTCACGTGACTGGGGAGGTACTTAAACA
CGGAAGTATCCTCAACCACGTAACTGGTCACGTGGTGCGCACGTCACGGC
AACCATTTTGTTTTACAAAATGGCGCATTTCCTTCCTCTTTTTTAAAAAT
TAACCGTTGGCGGCGGCGCGCGCGCTACGCGCGCGCGCCGGGGAGCTCTG
CCCCCCCCCGCGCATGCGCGCGGGTCCCCCCCCCGCGGGGGGCTCCGCCC
CCCGGTCCCCCCCCCG (SEQ ID NO: 47)
Annotations:

| Putative Domain | Base range |
|---|---|
| TATA Box | 82-86 |
| Initiator Element | 94-115 |
| Transcriptional Start Site | 115 |
| 5′ UTR Conserved Domain | 170-240 |
| ORF2 | 357-728 |
| ORF2/2 | 357-724; 2411-2870 |
| ORF2/3 | 357-724; 2646-3081 |
| ORF1 | 599-2896 |
| ORF1/1 | 599-724; 2411-2896 |
| ORF1/2 | 599-724; 2646-2870 |
| Three open-reading frame region | 2629-2867 |
| Poly(A) Signal | 3076-3086 |
| GC-rich region | 3759-3866 |

TABLE A13
Exemplary Anellovirus amino acid sequences (Alphatorquevirus, Clade 7)
TTV-HD16d (Alphatorquevirus Clade 7)

| | |
|---|---|
| ORF2 | MSWSRPVHNAAGIERQWFESTFRSHASCCGCGNFVNHINVLAARYGFTGGP |
| | TPPGGPGPRPQLRPALPAPDPDPQAPNREPWRGAGGGNDGEGAAGNPGGA |
| | AGDVYDGEDLDALFAAVVEDVE (SEQ ID NO: 48) |
| ORF2/2 | MSWSRPVHNAAGIERQWFESTFRSHASCCGCGNFVNHINVLAARYGFTGGP |
| | TPPGGPGPRPQLRPALPAPDPDPQAPNREPWRGAGGGNDGEGAAGNPGGA |
| | AGDVYDGEDLDALFAAVVEDVESSRTPAKTVDWNPPTPVDSVATYKLLTH |
| | TPWAPNSRSTTGTTDMAFLAKTLSTECLNNQKMMQTILTHTKGLDIFHPQT |
| | KPPKSKKKTSVSSKQHRRTQKRAIKKSSKKRKYSDSSQSSTSNSTCSSQSGSE |
| | SESNSDTYSNRCSKLRPIST (SEQ ID NO: 49) |
| ORF2/3 | MSWSRPVHNAAGIERQWFESTFRSHASCCGCGNFVNHINVLAARYGFTGGP |
| | TPPGGPGPRPQLRPALPAPDPDPQAPNREPWRGAGGGNDGEGAAGNPGGA |
| | AGDVYDGEDLDALFAAVVEDVEPSRPRARKRLQFPQNSTVELRRERSRSPP |
| | RNASTPIPARAAQATPPAARRAAANRRATPIPTPTDVQNSGQSPPKPIYIYPA |
| | VKQVFMFDPPGPKAISGAKAWEDEFLTAKVWNRPVRKYYSDTPYYPWAPK |
| | PQYSVSFKLGWK (SEQ ID NO: 50) |
| ORF1 | MAWSWWWQRWRRRRWKPRRRRWRRLRWRRPRRAVRRRRRGRRVRRRR |
| | WARRRGRRRRYATRRKRRYRGRRFKKKLVLTQWHPNTMRRCLIKGIVPLV |
| | ICGHTRWNYNYALHSKDYTEEGRYPHGGALSTTTWSLKVLYDEHLKHHDF |
| | WGYPNNQLDLARYKGAKFTFYRHKKTDFIIFFNRKPPFKLNKYSCASYHPG |
| | MLMQQRHKILLPSYETKPKGRPKITVRIKPPTLLEDKWYTQQDLCDVNLLQ |
| | LVVTAADFRHPLCSPQTNTPTTTFQVLKDIYYDTMSISEPTDSYTSVNNKSTT |
| | QTFTNYSNTLENILYTRASYWNSFHATEYLNPNIIYKNGEKLFKEHEDLITW |
| | MTQTNNTGFLTKNNTAFGNNSYRPNADKIKKARKTYWNALIGTNDLATNI |
| | GQARAERFEYHLGWYSPIFLSRHRSNMNFARAYQDVTYNPNCDRGVNNRV |
| | WVQPLTKPTTEFDEKRCKCVVQHLPLWAALYCYQDFVEEELGSSSEILNSC |
| | LLVLQCPYTFPPMYDKKLPDKGFVFYDSLFGDGKMSDGRGQVDIFWQQRW |
| | YPRLATQMQVMHDITMTGPFSYRDELVSTQLTAKYTFDFMWGGNMISTQII |
| | KNPCKDSGLEPAYPGRQRRDLQIVDPYSMGPQFSFHNWDYRHGLFGQDAID |
| | RVSKQPKDDADYPNPYKRPRYFPPTDQAAQEQEKDFSFLKTAPSNSEESDQE |
| | VLQETQVLRFQPEQHKQLHLQLAERQRIGEQLRYLLQQMFKTQANLHLNPY |
| | TFTQL (SEQ ID NO: 51) |
| ORF1/1 | MAWSWWWQRWRRRRWKPRRRRWRRLRWRRPRRAVRRRRRGRRIIKNPC |
| | KDSGLEPAYPGRQRRDLQIVDPYSMGPQFSFHNWDYRHGLFGQDAIDRVSK |
| | QPKDDADYPNPYKRPRYFPPTDQAAQEQEKDFSFLKTAPSNSEESDQEVLQE |
| | TQVLRFQPEQHKQLHLQLAERQRIGEQLRYLLQQMFKTQANLHLNPYTFTQ |
| | L (SEQ ID NO: 52) |
| ORF1/2 | MAWSWWWQRWRRRRWKPRRRRWRRLRWRRPRRAVRRRRRGRRTKPPK |
| | SKKKTSVSSKQHRRTQKRAIKKSSKKRKYSDSSQSSTSNSTCSSQSGSESESN |
| | SDTYSNRCSKLRPIST (SEQ ID NO: 53) |

TABLE N14
Exemplary Anellovirus nucleic acid sequence (Betatorquevirus)

| | |
|---|---|
| Name | Ring2 |
| Genus/Clade | Betatorquevirus |
| Accession Number | JX134045.1 |

Full Sequence: 2797 bp
1 10 20 30 40 50
| | | | | |
TAATAAATATTCAACAGGAAAACCACCTAATTTAAATTGCCGACCACAAA
CCGTCACTTAGTTCCCCTTTTTGCAACAACTTCTGCTTTTTTCCAACTGC
CGGAAAACCACATAATTTGCATGGCTAACCACAAACTGATATGCTAATTA
ACTTCCACAAAACAACTTCCCCTTTTAAAACCACACCTACAAATTAATTA
TTAAACACAGTCACATCCTGGGAGGTACTACCACACTATAATACCAAGTG
CACTTCCGAATGGCTGAGTTTATGCCGCTAGACGGAGAACGCATCAGTTA
CTGACTGCGGACTGAACTTGGGCGGGTGCCGAAGGTGAGTGAAACCACCG
AAGTCAAGGGGCAATTCGGGCTAGTTCAGTCTAGCGGAACGGGCAAGAAA
CTTAAAATTATTTTATTTTTCAGATGAGCGACTGCTTTAAACCAACATGC
TACAACAACAAAACAAAGCAAACTCACTGGATTAATAACCTGCATTTAAC
CCACGACCTGATCTGCTTCTGCCCAACACCAACTAGACACTTATTACTAG
CTTTAGCAGAACAACAAGAAACAATTGAAGTGTCTAAACAAGAAAAAGAA
AAAATAACAAGATGCCTTATTACTACAGAAGAAGACGGTACAACTACAGA
CGTCCTAGATGGTATGGACGAGGTTGGATTAGACGCCCTTTTCGCAGAAG
ATTTCGAAGAAAAAGAAGGGTAAGACCTACTTATACTACTATTCCTCTAA
AGCAATGGCAACCGCCATATAAAAGAACATGCTATATAAAAGGACAAGAC
TGTTTAATATACTATAGCAACTTAAGACTGGGAATGAATAGTACAATGTA
TGAAAAAAGTATTGTACCTGTACATTGGCCGGGAGGGGGTTCTTTTTCTG
TAAGCATGTTAACTTTAGATGCCTTGTATGATATACATAAACTTTGTAGA
AACTGGTGGACATCCACAAACCAAGACTTACCACTAGTAAGATATAAAGG
ATGCAAAATAACATTTTATCAAAGCACATTTACAGACTACATAGTAAGAA
TACATACAGAACTACCAGCTAACAGTAACAAACTAACATACCCAAACACA
CATCCACTAATGATGATGATGTCTAAGTACAAACACATTATACCTAGTAG
ACAAACAAGAAGAAAAAAGAAACCATACACAAAAATATTTGTAAAACCAC
CTCCGCAATTTGAAAACAAATGGTACTTTGCTACAGACCTCTACAAAATT
CCATTACTACAAATACACTGCACAGCATGCAACTTACAAAACCCATTTGT
AAAACCAGACAAATTATCAAACAATGTTACATTATGGTCACTAAACACCA
TAAGCATACAAAATAGAAACATGTCAGTGGATCAAGGACAATCATGGCCA
TTTAAAATACTAGGAACACAAAGCTTTTATTTTTACTTTTACACCGGAGC
AAACCTACCAGGTGACACAACACAAATACCAGTAGCAGACCTATTACCAC
TAACAAACCCAAGAATAAACAGACCAGGACAATCACTAAATGAGGCAAAA
ATTACAGACCATATTACTTTCACAGAATACAAAAACAAATTTACAAATTA
TTGGGGTAACCCATTTAATAAACACATTCAAGAACACCTAGATATGATAC
TATACTCACTAAAAAGTCCAGAAGCAATAAAAAACGAATGGACAACAGAA
AACATGAAATGGAACCAATTAAACAATGCAGGAACAATGGCATTAACACC
ATTTAACGAGCCAATATTCACACAAATACAATATAACCCAGATAGAGACA
CAGGAGAAGACACTCAATTATACCTACTCTCTAACGCTACAGGAACAGGA
TGGGACCCACCAGGAATTCCAGAATTAATACTAGAAGGATTTCCACTATG
GTTAATATATTGGGGATTTGCAGACTTTCAAAAAAACCTAAAAAAAGTAA
CAAACATAGACACAAATTACATGTTAGTAGCAAAAACAAAATTTACACAA
AAACCTGGCACATTCTACTTAGTAATACTAAATGACACCTTTGTAGAAGG
CAATAGCCCATATGAAAAACAACCTTTACCTGAAGACAACATTAAATGGT
ACCCACAAGTACAATACCAATTAGAAGCACAAAACAAACTACTACAAACT
GGGCCATTTACACCAAACATACAAGGACAACTATCAGACAATATATCAAT
GTTTTATAAATTTTACTTTAAATGGGGAGGAAGCCCACCAAAAGCAATTA
ATGTTGAAAATCCTGCCCACCAGATTCAATATCCCATACCCCGTAACGAG
CATGAAACAACTTCGTTACAGAGTCCAGGGGAAGCCCCAGAATCCATCTT
ATACTCCTTCGACTATAGACACGGGAACTACACAACAACAGCTTTGTCAC
GAATTAGCCAAGACTGGGCACTTAAAGACACTGTTTCTAAAATTACAGAG
CCAGATCGACAGCAACTGCTCAAACAAGCCCTCGAATGCCTGCAAATCTC
GGAAGAAACGCAGGAGAAAAAAGAAAAAGAAGTACAGCAGCTCATCAGCA
ACCTCAGACAGCAGCAGCAGCTGTACAGAGAGCGAATAATATCATTATTA
AAGGACCAATAACTTTTAACTGTGTAAAAAAGGTGAAATTGTTTGATGAT
AAACCAAAAAACCGTAGATTTACACCTGAGGAATTTGAAACTGAGTTACA
AATAGCAAAATGGTTAAAGAGACCCCCAAGATCCTTTGTAAATGATCCTC
CCTTTTACCCATGGTTACCACCTGAACCTGTTGTAAACTTTAAGCTTAAT
TTTACTGAATAAAGGCCAGCATTAATTCACTTAAGGAGTCTGTTTATTTA
AGTTAAACCTTAATAAACGGTCACCGCCTCCCTAATACGCAGGCGCAGAA
AGGGGGCTCCGCCCCCTTTAACCCCCAGGGGGCTCCGCCCCCTGAAACCC
CCAAGGGGGCTACGCCCCCTTACACCCCC (SEQ ID NO: 54)
Annotations:

| Putative Domain | Base range |
|---|---|
| TATA Box | 237-243 |
| Cap Site | 260-267 |
| Transcriptional Start Site | 267 |
| 5′ UTR Conserved Domain | 323-393 |
| ORF2 | 424-723 |
| ORF2/2 | 424-719; 2274-2589 |
| ORF2/3 | 424-719; 2449-2812 |
| ORF1 | 612- 2612 |
| ORF1/1 | 612-719; 2274-2612 |
| ORF1/2 | 612-719; 2449-2589 |
| Three open-reading frame region | 2441-2586 |
| Poly(A) Signal | 2808-2813 |
| GC-rich region | 2868-2929 |

TABLE A14
Exemplary Anellovirus amino acid sequences (Betatorquevirus)
Ring2 (Betatorquevirus)

| | |
|---|---|
| ORF2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEG (SEQ ID NO: 55) |
| ORF2/2 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGFNIPYPVTSMKQLRY |
| | RVQGKPQNPSYTPSTIDTGTTQQQLCHELAKTGHLKTLFLKLQSQIDSNCSNKPSNA |
| | CKSRKKRRRKKKKKYSSSSATSDSSSSCTESE (SEQ ID NO: 56) |
| ORF2/3 | MSDCFKPTCYNNKTKQTHWINNLHLTHDLICFCPTPTRHLLLALAEQQETIEVSKQE |
| | KEKITRCLITTEEDGTTTDVLDGMDEVGLDALFAEDFEEKEGARSTATAQTSPRMP |
| | ANLGRNAGEKRKRSTAAHQQPQTAAAAVQRANNIIIKGPITFNCVKKVKLFDDKPK |
| | NRRFTPEEFETELQIAKWLKRPPRSFVNDPPFYPWLPPEPVVNFKLNFTE (SEQ ID |
| | NO: 57) |
| ORF1 | MPYYYRRRRYNYRRPRWYGRGWIRRPFRRRFRRKRRVRPTYTTIPLKQWQPPYKR |
| | TCYIKGQDCLIYYSNLRLGMNSTMYEKSIVPVHWPGGGSFSVSMLTLDALYDIHKL |
| | CRNWWTSTNQDLPLVRYKGCKITFYQSTFTDYIVRIHTELPANSNKLTYPNTHPLM |
| | MMMSKYKHIIPSRQTRRKKKPYTKIFVKPPPQFENKWYFATDLYKIPLLQIHCTACN |
| | LQNPFVKPDKLSNNVTLWSLNTISIQNRNMSVDQGQSWPFKILGTQSFYFYFYTGA |
| | NLPGDTTQIPVADLLPLTNPRINRPGQSLNEAKITDHITFTEYKNKFTNYWGNPFNK |
| | HIQEHLDMILYSLKSPEAIKNEWTTENMKWNQLNNAGTMALTPFNEPIFTQIQYNP |
| | DRDTGEDTQLYLLSNATGTGWDPPGIPELILEGFPLWLIYWGFADFQKNLKKVTNID |
| | TNYMLVAKTKFTQKPGTFYLVILNDTFVEGNSPYEKQPLPEDNIKWYPQVQYQLEA |
| | QNKLLQTGPFTPNIQGQLSDNISMFYKFYFKWGGSPPKAINVENPAHQIQYPIPRNE |
| | HETTSLQSPGEAPESILYSFDYRHGNYTTTALSRISQDWALKDTVSKITEPDRQQLLK |
| | QALECLQISEETQEKKEKEVQQLISNLRQQQQLYRERIISLLKDQ (SEQ ID NO: 58) |
| ORF1/1 | MPYYYRRRRYNYRRPRWYGRGWIRRPFRRRFRRKRRIQYPIPRNEHETTSLQSPGE |
| | APESILYSFDYRHGNYTTTALSRISQDWALKDTVSKITEPDRQQLLKQALECLQISEE |
| | TQEKKEKEVQQLISNLRQQQQLYRERIISLLKDQ (SEQ ID NO: 59) |
| ORF1/2 | MPYYYRRRRYNYRRPRWYGRGWIRRPFRRRFRRKRRSQIDSNCSNKPSNACKSRK |
| | KRRRKKKKKYSSSSATSDSSSSCTESE (SEQ ID NO: 60) |

TABLE N15
Exemplary Anellovirus nucleic acid sequence (Gammatorquevirus)

| | |
|---|---|
| Name | TTMDV-MD1-073 |
| Genus/Clade | Gammatorquevirus |
| Accession Number | AB290918.1 |

Full Sequence: 3242 bp
1 10 20 30 40 50
| | | | | |
AGGTGGAGACTCTTAAGCTATATAACCAAGTGGGGTGGCGAATGGCTGAG
TTTACCCCGCTAGACGGTGCAGGGACCGGATCGAGCGCAGCGAGGAGGTC
CCCGGCTGCCCGTGGGCGGGAGCCCGAGGTGAGTGAAACCACCGAGGTCT
AGGGGCAATTCGGGCTAGGGCAGTCTAGCGGAACGGGCAAGAAACTTAAA
AATATTTCTTTTACAGATGCAAAACCTATCAGCCAAAGACTTCTACAAAC
CATGCAGATACAACTGTGAAACTAAAAACCAAATGTGGATGTCTGGCATT
GCTGACTCCCATGACAGTTGGTGTGACTGTGATACTCCTTTTGCTCACCT
CCTGGCTAGTATTTTTCCTCCTGGTCACACAGATCGCACACGAACCATCC
AAGAAATACTTACCAGAGATTTTAGGAAAACATGCCTTTCTGGTGGGGCC
GACGCAACAAATTCTGGTATGGCCGAAACTATAGAAGAAAAAAGAGAAGA
TTTCCAAAAAGAAGAAAAAGAAGATTTTACAGAAGAACAAAATATAGAAG
ACCTGCTCGCCGCCGTCGCAGACGCAGAAGGAAGGTAAGAAGAAAAAAAA
AAACTCTTATAGTAAGACAATGGCAGCCAGACTCTATTGTACTCTGTAAA
ATTAAAGGGTATGACTCTATAATATGGGGAGCTGAAGGCACACAGTTTCA
ATGTTCTACACATGAAATGTATGAATATACAAGACAAAAGTACCCTGGGG
GAGGAGGATTTGGTGTACAACTTTACAGCTTAGAGTATTTGTATGACCAA
TGGAAACTTAGAAATAATATATGGACTAAAACAAATCAACTCAAAGATTT
GTGTAGATACTTAAAATGTGTTATGACCTTTTACAGACACCAACACATAG
ATTTTGTAATTGTATATGAAAGACAACCCCCATTTGAAATAGATAAACTA
ACATACATGAAATATCATCCATATATGTTATTACAAAGAAAGCATAAAAT
AATTTTACCTAGTCAAACAACTAATCCTAGAGGTAAATTAAAAAAAAAGA
AAACTATTAAACCTCCCAAACAAATGCTCAGCAAATGGTTTTTTCAACAA
CAATTTGCTAAATATGATCTACTACTTATTGCTGCAGCAGCATGTAGTTT
AAGATACCCTAGAATAGGCTGCTGCAATGAAAATAGAATGATAACCTTAT
ACTGTTTAAATACTAAATTTTATCAAGATACAGAATGGGGAACTACAAAA
CAGGCCCCCCACTACTTTAAACCATATGCAACAATTAATAAATCCATGAT
ATTTGTCTCTAACTATGGAGGTAAAAAAACAGAATATAACATAGGCCAAT
GGATAGAAACAGATATACCTGGAGAAGGTAATCTAGCAAGATACTACAGA
TCAATAAGTAAAGAAGGAGGTTACTTTTCACCTAAAATACTGCAAGCATA
TCAAACAAAAGTAAAGTCTGTAGACTACAAACCTTTACCAATTGTTTTAG
GTAGATATAACCCAGCAATAGATGATGGAAAAGGCAACAAAATTTACTTA
CAAACTATAATGAATGGCCATTGGGGCCTACCTCAAAAAACACCAGATTA
TATAATAGAAGAGGTCCCTCTTTGGCTAGGCTTCTGGGGATACTATAACT
ACTTAAAACAAACAAGAACTGAAGCTATATTTCCACTACACATGTTTGTA
GTGCAAAGCAAATACATTCAAACACAACAAACAGAAACACCTAACAATTT
TTGGGCATTTATAGACAACAGCTTTATACAGGGCAAAAACCCATGGGACT
CAGTTATTACTTACTCAGAACAAAAGCTATGGTTTCCTACAGTTGCATGG
CAACTAAAAACCATAAATGCTATTTGTGAAAGTGGACCATATGTACCTAA
ACTAGACAATCAAACATATAGTACCTGGGAACTAGCAACTCATTACTCAT
TTCACTTTAAATGGGGTGGTCCACAGATATCAGACCAACCAGTTGAAGAC
CCAGGAAACAAAAACAAATATGATGTGCCCGATACAATCAAAGAAGCATT
ACAAATTGTTAACCCAGCAAAAAACATTGCTGCCACGATGTTCCATGACT
GGGACTACAGACGGGGTTGCATTACATCAACAGCTATTAAAAGAATGCAA
CAAAACCTCCCAACTGATTCATCTCTCGAATCTGATTCAGACTCAGAACC
AGCACCCAAGAAAAAAAGACTACTACCAGTCCTCCACGACCCACAAAAGA
AAACGGAAAAGATCAACCAATGTCTCCTCTCTCTCTGCGAAGAAAGTACA
TGCCAGGAGCAGGAAACGGAGGAAAACATCCTCAAGCTCATCCAGCAGCA
GCAGCAGCAGCAGCAGAAACTCAAGCACAACCTCTTAGTACTAATCAAGG
ACTTAAAAGTGAAACAAAGATTATTACAACTACAAACGGGGGTACTAGAA
TAACCCTTACCAGATTTAAACCAGGATTTGAGCAAGAAACTGAAAAAGAG
TTAGCACAAGCATTTAACAGACCCCCTAGACTGTTCAAAGAAGATAAACC
CTTTTACCCCTGGCTACCCAGATTTACACCCCTTGTAAACTTTCACCTTA
ATTTTAAAGGCTAGGCCTACACTGCTCACTTAGTGGTGTATGTTTATTAA
AGTTTGCACCCCAGAAAAATTGTAAAATAAAAAAAAAAAAAAAAAATAAA
AAATTGCAAAAATTCGGCGCTCGCGCGCGCTGCGCGCGCGAGCGCCGTCA
CGCGCCGGCGCTCGCGCGCCGCGCGTATGTGCTAACACACCACGCACCTA
GATTGGGGTGCGCGCGTAGCGCGCGCACCCCAATGCGCCCCGCCCTCGTT
CCGACCCGCTTGCGCGGGTCGGACCACTTCGGGCTCGGGGGGGCGCGCCT
GCGGCGCTTATTTACTAAACAGACTCCGAGTCGCCATTGGGCCCCCCCTA
AGCTCCGCCCCCCTCATGAATATTCATAAAGGAAACCACAAAATTAGAAT
TGCCGACCACAAACTGCCATATGCTAATTAGTTCCCCTTTTACACAGTAA
AAAGGGGAAGTGGGGGGGCAGAGCCCCCCCACACCCCCCGCGGGGGGGGC
AGAGCCCCCCCCGCACCCCCCCTACGTCACAGGCCACGCCCCCGCCGCCA
TCTTGGGTGCGGCAGGGCGGGGACTAAAATGGCGGGACCCAATCATTTTA
TACTTTCACTTTCCAATTAAAACCCGCCACGTCACACAAAAG (SEQ ID NO: 61)
Annotations:

| Putative Domain | Base range |
|---|---|
| TATA Box | 21-25 |
| Cap Site | 42-49 |
| Transcriptional Start Site | 49 |
| 5′ UTR Conserved Domain | 117-187 |
| ORF2 | 283-588 |
| ORF2/2 | 283-584; 1977-2388 |
| ORF2/3 | 283-584; 2197-2614 |
| ORF1 | 432-2453 |
| ORF1/1 | 432-584; 1977-2453 |
| ORF1/2 | 432-584; 2197-2388 |
| Three open-reading frame region | 2186-2385 |
| Poly(A) Signal | 2676-2681 |
| GC-rich region | 3054-3172 |

TABLE A15
Exemplary Anellovirus amino acid sequences (Gammatorquevirus)
TTMDV-MD1-073 (Gammatorquevirus)

| | |
|---|---|
| ORF2 | MWMSGIADSHDSWCDCDTPFAHLLASIFPPGHTDRTRTIQEILTRDFRKTCLSGGAD |
| | ATNSGMAETIEEKREDFQKEEKEDFTEEQNIEDLLAAVADAEGR (SEQ ID NO: 62) |
| ORF2/2 | MWMSGIADSHDSWCDCDTPFAHLLASIFPPGHTDRTRTIQEILTRDFRKTCLSGGAD |
| | ATNSGMAETIEEKREDFQKEEKEDFTEEQNIEDLLAAVADAEGRYQTNQLKTQETK |
| | TNMMCPIQSKKHYKLLTQQKTLLPRCSMTGTTDGVALHQQLLKECNKTSQLIHLSN |
| | LIQTQNQHPRKKDYYQSSTTHKRKRKRSTNVSSLSAKKVHARSRKRRKTSSSSSSSS |
| | SSSSRNSSTTS (SEQ ID NO: 63) |
| ORF2/3 | MWMSGIADSHDSWCDCDTPFAHLLASIFPPGHTDRTRTIQEILTRDFRKTCLSGGAD |
| | ATNSGMAETIEEKREDFQKEEKEDFTEEQNIEDLLAAVADAEGRTSTQEKKTTTSPP |
| | RPTKENGKDQPMSPLSLRRKYMPGAGNGGKHPQAHPAAAAAAAETQAQPLSTNQ |
| | GLKSETKIITTTNGGTRITLTRFKPGFEQETEKELAQAFNRPPRLFKEDKPFYPWLPRF |
| | TPLVNFHLNFKG (SEQ ID NO: 64) |
| ORF1 | MPFWWGRRNKFWYGRNYRRKKRRFPKRRKRRFYRRTKYRRPARRRRRRRRKVR |
| | RKKKTLIVRQWQPDSIVLCKIKGYDSIIWGAEGTQFQCSTHEMYEYTRQKYPGGGG |
| | FGVQLYSLEYLYDQWKLRNNIWTKTNQLKDLCRYLKCVMTFYRHQHIDFVIVYER |
| | QPPFEIDKLTYMKYHPYMLLQRKHKIILPSQTTNPRGKLKKKKTIKPPKQMLSKWFF |
| | QQQFAKYDLLLIAAAACSLRYPRIGCCNENRMITLYCLNTKFYQDTEWGTTKQAPH |
| | YFKPYATINKSMIFVSNYGGKKTEYNIGQWIETDIPGEGNLARYYRSISKEGGYFSPK |
| | ILQAYQTKVKSVDYKPLPIVLGRYNPAIDDGKGNKIYLQTIMNGHWGLPQKTPDYII |
| | EEVPLWLGFWGYYNYLKQTRTEAIFPLHMFVVQSKYIQTQQTETPNNFWAFIDNSFI |
| | QGKNPWDSVITYSEQKLWFPTVAWQLKTINAICESGPYVPKLDNQTYSTWELATH |
| | YSFHFKWGGPQISDQPVEDPGNKNKYDVPDTIKEALQIVNPAKNIAATMFHDWDY |
| | RRGCITSTAIKRMQQNLPTDSSLESDSDSEPAPKKKRLLPVLHDPQKKTEKINQCLLS |
| | LCEESTCQEQETEENILKLIQQQQQQQQKLKHNLLVLIKDLKVKQRLLQLQTGVLE |
| | (SEQ ID NO: 65) |
| ORF1/1 | MPFWWGRRNKFWYGRNYRRKKRRFPKRRKRRFYRRTKYRRPARRRRRRRRKISD |
| | QPVEDPGNKNKYDVPDTIKEALQIVNPAKNIAATMFHDWDYRRGCITSTAIKRMQQ |
| | NLPTDSSLESDSDSEPAPKKKRLLPVLHDPQKKTEKINQCLLSLCEESTCQEQETEEN |
| | ILKLIQQQQQQQQKLKHNLLVLIKDLKVKQRLLQLQTGVLE (SEQ ID NO: 66) |
| ORF1/2 | MPFWWGRRNKFWYGRNYRRKKRRFPKRRKRRFYRRTKYRRPARRRRRRRRKISD |
| | QPVEDPGNKNKYDVPDTIKEALQIVNPAKNIAATMFHDWDYRRGCITSTAIKRMQQ |
| | NLPTDSSLESDSDSEPAPKKKRLLPVLHDPQKKTEKINQCLLSLCEESTCQEQETEEN |
| | ILKLIQQQQQQ QQKLKHNLLVLIKDLKVKQRLLQLQTGVLE (SEQ ID NO: 67) |

TABLE N16
Exemplary Anellovirus nucleic acid sequence (Gammatorquevirus)

| | |
|---|---|
| Name | Ring3.1 |
| Genus/Clade | Gammatorquevirus |
| Accession Number | |

Full Sequence: 3264 bp
1 10 20 30 40 50
| | | | | |
TAAAATGGCGGCAACCAATCATTTTATACTTTCACTTTCCAATTACAAGC
CGCCACGTCACAGAACAGGGGTGGAGACTTTAAAACTATATAACCAAGTG
ATGTGACGAATGGCTGAGTTTACCCCGCTAGACGGTGCAGGGACCGGATC
GAGCGCAGCGAGGAGGTCCCCGGCTGCCCGTGGGCGGGAGCCCGAGGTGA
GTGAAACCACCGAGGTCTAGGGGCAATTCGGGCTAGGGCAGTCTAGCGGA
ACGGGCAAGAAACTTAAAATATGTTTTGTTTCAGATGCAGACACCTGCTT
CACAGATAAGCTCAGACGACTTCTTTGTACACACTCCATTTAATGCAGTA
ACTAAACAGCAAATATGGATGTCTCAAATTGCTGATGGACATGACAACAT
TTGTCACTGCCACCGTCCTTTTGCTCACCTGCTTGCTAATATTTTTCCTC
CTGGTCATAAAGACAGGGATCTTACCATTAATCAAATACTTGCTAGAGAT
CTTACAGAAACATGCCATTCTGGTGGAGACGAAGGAACAAGCGGTGGTGG
GGTCGCCGCTTCCGCTACCGCCGCTACAACAAATATAAAACCAGAAGGAG
ACGCAGAATACCCAGAAGACGAAATAGAAGATTTACTAAGACACGCAGGA
GAAGAAAAAGAAAGAAGGTAAGAAGAAAACTTAAAAAAATTACTATTAAA
CAATGGCAGCCAGATTCAGTGAAAAAATGTAAAATTAAAGGATATAGTAC
TTTAGTTATGGGTGCACAAGGAAAACAATACAACTGTTACACAAACCAAG
CAAGTGACTATGTTCAGCCTAAAGCACCACAAGGTGGGGGCTTTGGCTGT
GAAGTATTTAATTTAAAATGGCTATACCAAGAATATACTGCACACAGAAA
TATTTGGACAAAAACAAATGAATATACAGACCTTTGTAGATACACTGGAG
CTCAAATAATTTTATACAGGCACCCAGATGTTGATTTTATAGTCAGCTGG
GACAATCAGCCACCTTTTTTACTTAACAAATATACATATCCAGAACTGCA
ACCACAAAACCTTTTACTAGCTAGAAGGAAAAGAATTATTCTTAGTCAAA
AATCAAACCCCAAAGGAAAACTAAGAATTAAACTAAGAATACCACCACCA
AAACAAATGATAACAAAATGGTTTTTTCAAAGAGACTTTTGTGATGTGAA
TCTGTTTAAACTATGTGCTTCTGCTGCTTCTTTCCGCTACCCAGGTATCA
GTCATGGAGCTCAAAGTACTATTTTTTCTGCATATGCTTTAAACACTGAC
TTTTATCAATGCAGTGACTGGTGCCAAACTAACACAGAAACTGGCTACCT
AAACATTAAAACACAACAAATGCCACTATGGTTTCATTACAGAGAGGGTG
GCAAAGAGAAATGGTATAAATACACCAACAAAGAACACAGACCATATACA
AATACATATCTTAAAAGTATTAGCTATAATGATGGATTGTTTTCTCCTAA
AGCCATGTTTGCATTTGAAGTAAAAGCGGGGGGTGAAGGAACAACAGAAC
CACCACAAGGCGCCCAATTAATTGCTAACCTTCCACTCATTGCACTAAGA
TATAATCCACATGAAGACACAGGCCATGGCAATGAAATTTACCTTACATC
AACTTTTAAAGGTACATATGACAAACCTAAAGTTACTGATGCTCTATACT
TTAACAATGTACCCCTGTGGATGGGATTTTATGGCTACTGGGACTTTATA
TTACAAGAAACAAAAAACAAAGGTGTCTTTGATCAACATATGTTTGTTGT
TAAATGTCCTGCCTTAAGGCCCATATCACAAGTCACAAAACAAGTATACT
ACCCACTTGTAGACATGGACTTTTGTTCAGGGAGACTGCCATTTGATGAA
TATTTATCCAAAGACATTAAAAGTCATTGGTATCCCACTGCAGAAAGACA
AACAGTTACAATAAATAATTTTGTTACAGCAGGTCCATACATGCCTAAAT
TTGAACCCACAGACAAAGACAGTACATGGCAATTAAACTATCACTATAAA
TTTTTTTTTAAGTGGGGTGGTCCACAAGTCACAGACCCAACTGTTGAAGA
CCCATGCAGCAGAAACAAATATCCTGTCCCCGATACAATGCAACAAACAA
TACAAATTAAAAACCCTGAAAAGCTGCACCCAGCAACCCTCTTCCATGAC
TGGGACCTTAGAAGGGGCTTCATTACACAAGCAGCTATTAAAAGAATGTC
AGAAAACCTCCAAATTGATTCATCTTTCGAATCTGATGGCACAGAATCAC
CCAAAAAAAAGAAAAGATGCACCAAAGAAATCCCAACACAAAACCAAAAG
CAAGAAGAGATCCAAGAATGTCTCCTCTCACTCTGCGAAGAGCCTACATG
CCAAGAAGAAACAGAGGACCTCCAGCTCTTCATCCAGCAGCAGCAGCAGC
AGCAGTACAAGCTCAGAAAAAACCTCTTCAAACTCCTCACTCACCTGAAA
AAAGGACAGAGAATAAGTCAACTACAAACGGGACTTTTAGAGTAATACCA
TTTAAACCAGGTTTTGAACAAGAAACAGAAAAAGAACTTGCCATAGCTTT
CTGCAGACCACCTAGAAAATATAAAAATGATCCCCCTTTTTATCCCTGGT
TACCATGGACACCCCTTGTACACTTTAACCTTAATTACAAAGGCTAGGCC
AACACTGTTCACTTAGTGGTGTATGTTTAATAAAGTTTCACCCCCAAAAA
AAAAAAAAAAAAAAAAAAAAAAAAAAAAAATAAAAAATTGCAAAAATTCG
GCGCTCGCGCGCGCTGCGCGCGCGCGAGCGCCGTCACGCGCCGGCGCTCG
CGCGCCGCGCGTATGTGCTAACACACCACGCACCTAGATTGGGGTGCGCG
CGCTAGCGCGCGCACCCCAATGCGCCCCGCCCTCGTTCCGACCCGCTTGC
GCGGGTCGGACCACTTCGGGCTCGGGGGGGCGCGCCTGCGGCGCTTTTTT
ACTAAACAGACTCCGAGCCGCCATTTGGCCCCCCCTAAGCTCCGCCCCCC
TCATGAATATTCATAAAGGAAACCACATAATTAGAATTGCCGACCACAAA
CTGCCATATGCTAATTAGTTCCCCTTTTACACAGTAAAAAGGGGAAGTGG
GGGGGCATAGCCCCCCCACACCCCCCGCGGGGGGGGCAGAGCCCCCCCCC
GCACCCCCCCCCTACGTCACAATCCACGCCCCCGCCGCCATCTTGGGTGC
GGCAGGGCGGGGGC (SEQ ID NO: 878)
Annotations:

| Putative Domain | Base range |
|---|---|
| TATA Box | 87-93 |
| Cap Site | 110-117 |
| Transcriptional Start Site | 117 |
| 5′ UTR Conserved Domain | 185-255 |
| ORF2 | 285-671 |
| ORF2/2 | 285-667; 2063-2498 |
| ORF2/3 | 285-667; 2295-2697 |
| TAIP | 385-585 |
| ORF1 | 512-2545 |
| ORF1/1 | 512-667; 2063-2545 |
| ORF1/2 | 512-667; 2295-2498 |
| Three open-reading frame region | 2295-2495 |
| Poly(A) Signal | 2729-2734 |
| GC-rich region | 3141-3264 |

TABLE A16
Exemplary Anellovirus amino acid sequences (Gammatorquevirus)
Ring 3.1 (Gammatorquevirus)

| | |
|---|---|
| ORF2 | MQTPASQISSDDFFVHTPFNAVTKQQIWMSQIADGHDNICHCHRPFAHLLAN |
| | IFPPGHKDRDLTINQ |
| | ILARDLTETCHSGGDEGTSGGGVAASATAATTNIKPEGDAEYPEDEIEDLLR |
| | HAGEEKERR (SEQ ID NO: 879) |
| ORF2/2 | MQTPASQISSDDFFVHTPFNAVTKQQIWMSQIADGHDNICHCHRPFAHLLAN |
| | IFPPGHKDRDLTINQ |
| | ILARDLTETCHSGGDEGTSGGGVAASATAATTNIKPEGDAEYPEDEIEDLLR |
| | HAGEEKERSGVVHKSQTQLLKTHAAETNILSPIQCNKQYKLKTLKSCTQQPS |
| | SMTGTLEGASLHKQLLKECQKTSKLIHLSNLMAQNHPKKRKDAPKKSQHK |
| | TKSKKRSKNVSSHSAKSLHAKKKQRTSSSSSSSSSSSSTSSEKTSSNSSLT |
| | (SEQ ID NO: 880) |
| ORF2/3 | MQTPASQISSDDFFVHTPFNAVTKQQIWMSQIADGHDNICHCHRPFAHLLAN |
| | IFPPGHKDRDLTINQ |
| | ILARDLTETCHSGGDEGTSGGGVAASATAATTNIKPEGDAEYPEDEIEDLLR |
| | HAGEEKERRITQKKEKMHQRNPNTKPKARRDPRMSPLTLRRAYMPRRNRG |
| | PPALHPAAAAAAVQAQKKPLQTPHSPEKRTENKSTTNGTFRVIPFKPGFEQE |
| | TEKELAIAFCRPPRKYKNDPPFYPWLPWTPLVHFNLNYKG (SEQ ID NO: 881) |
| TAIP | MDMTTFVTATVLLLTCLLIFFLLVIKTGILPLIKYLLEILQKHAILVETKEQAV |
| | VGSPLPLPPLQQI (SEQ ID NO: 882) |
| ORF1 | MPFWWRRRNKRWWGRRFRYRRYNKYKTRRRRRIPRRRNRRFTKTRRRRK |
| | RKKVRRKLKKITIKQWQP |
| | DSVKKCKIKGYSTLVMGAQGKQYNCYTNQASDYVQPKAPQGGGFGCEVF |
| | NLKWLYQEYTAHRNIWTKTNEYTDLCRYTGAQIILYRHPDVDFIVSWDNQP |
| | PFLLNKYTYPELQPQNLLLARRKRIILSQKSNPKGKLRIKLRIPPPKQMITKWF |
| | FQRDFCDVNLFKLCASAASFRYPGISHGAQSTIFSAYALNTDFYQCSDWCQT |
| | NTETGYLNIKTQQMPLWFHYREGGKEKWYKYTNKEHRPYTNTYLKSISYN |
| | DGLFSPKAMFAFEVKAGGEGTTEPPQGAQLIANLPLIALRYNPHEDTGHGNE |
| | IYLTSTFKGTYDKPKVTDALYFNNVPLWMGFYGYWDFILQETKNKGVFDQ |
| | HMFVVKCPALRPISQVTKQVYYPLVDMDFCSGRLPFDEYLSKDIKSHWYPT |
| | AERQTVTINNFVTAGPYMPKFEPTDKDSTWQLNYHYKFFFKWGGPQVTDP |
| | TVEDPCSRNKYPVPDTMQQTIQIKNPEKLHPATLFHDWDLRRGFITQAAIKR |
| | MSENLQIDSSFESDGTESPKKKKRCTKEIPTQNQKQEEIQECLLSLCEEPTCQ |
| | EETEDLQLFIQQQQQQQYKLRKNLFKLLTHLKKGQRISQLQTGLLE (SEQ ID |
| | NO: 883) |
| ORF1/1 | MPFWWRRRNKRWWGRRFRYRRYNKYKTRRRRRIPRRRNRRFTKTRRRRK |
| | RKKWGGPQVTDPTVEDPCSRNKYPVPDTMQQTIQIKNPEKLHPATLFHDWD |
| | LRRGFITQAAIKRMSENLQIDSSFESDGTESPKKKKRCTKEIPTQNQKQEEIQE |
| | CLLSLCEEPTCQEETEDLQLFIQQQQQQQYKLRKNLFKLLTHLKKGQRISQL |
| | QTGLLE (SEQ ID NO: 884) |
| ORF1/2 | MPFWWRRRNKRWWGRRFRYRRYNKYKTRRRRRIPRRRNRRFTKTRRRRK |
| | RKKNHPKKRKDAPKKSQHKTKSKKRSKNVSSHSAKSLHAKKKQRTSSSSSS |
| | SSSSSSTSSEKTSSNSSLT (SEQ ID NO: 885) |

TABLE N17
Exemplary Anellovirus nucleic acid sequence (Gammatorquevirus)

| | |
|---|---|
| Name | Ring4 |
| Genus/Clade | Gammatorquevirus |
| Accession Number | |

Full Sequence: 3176 bp
1 10 20 30 40 50
| | | | | |
TAAAATGGCGGGAGCCAATCATTTTATACTTTCACTTTCCAATTAAAAAT
GGCCACGTCACAAACAAGGGGTGGAGCCATTTAAACTATATAACTAAGTG
GGGTGGCGAATGGCTGAGTTTACCCCGCTAGACGGTGCAGGGACCGGATC
GAGCGCAGCGAGGAGGTCCCCGGCTGCCCATGGGCGGGAGCCGAGGTGAG
TGAAACCACCGAGGTCTAGGGGCAATTCGGGCTAGGGCAGTCTAGCGGAA
CGGGCAAGAAACTTAAAACAATATTTGTTTTACAGATGGTTAGTATATCC
TCAAGTGATTTTTTTAAGAAAACGAAATTTAATGAGGAGACGCAGAACCA
AGTATGGATGTCTCAAATTGCTGACTCTCATGATAATATCTGCAGTTGCT
GGCATCCATTTGCTCACCTTCTTGCTTCCATATTTCCTCCTGGCCACAAA
GATCGTGATCTTACTATTAACCAAATTCTTCTAAGAGATTATAAAGAAAA
ATGCCATTCTGGTGGAGAAGAAGGAGAAAATTCTGGACCAACAACAGGTT
TAATTACACCAAAAGAAGAAGATATAGAAAAAGATGGCCCAGAAGGCGCC
GCAGAAGAAGACCATACAGACGCCCTGTTCGCCGCCGCCGTAGAAAACTT
CGAAAGGTAAAGAGAAAAAAAAAATCTTTAATTGTTAGACAATGGCAACC
AGACAGTATAAGAACTTGTAAAATTATAGGACAGTCAGCTATAGTTGTTG
GGGCTGAAGGAAAGCAAATGTACTGTTATACTGTCAATAAGTTAATTAAT
GTGCCCCCAAAAACACCATATGGGGGAGGCTTTGGAGTAGACCAATACAC
ACTGAAATACTTATATGAAGAATACAGATTTGCACAAAACATTTGGACAC
AATCTAATGTACTGAAAGACTTATGCAGATACATAAATGTTAAGCTAATA
TTCTACAGAGACAACAAAACAGACTTTGTCCTTTCCTATGACAGAAACCC
ACCTTTTCAACTAACAAAATTTACATACCCAGGAGCACACCCACAACAAA
TCATGCTTCAAAAACACCACAAATTCATACTATCACAAATGACAAAGCCT
AATGGAAGACTAACAAAAAAACTCAAAATTAAACCTCCTAAACAAATGCT
TTCTAAATGGTTCTTTTCAAAACAATTCTGTAAATACCCTTTACTATCTC
TTAAAGCTTCTGCACTAGACCTTAGGCACTCTTACCTAGGCTGCTGTAAT
GAAAATCCACAGGTATTTTTTTATTATTTAAACCATGGATACTACACAAT
AACAAACTGGGGAGCACAATCCTCAACAGCATACAGACCTAACTCCAAGG
TGACAGACACAACATACTACAGATACAAAAATGACAGAAAAAATATTAAC
ATTAAAAGCCATGAATACGAAAAAAGTATATCATATGAAAACGGTTATTT
TCAATCTAGTTTCTTACAAACACAGTGCATATATACCAGTGAGCGTGGTG
AAGCCTGTATAGCAGAAAAACCACTAGGAATAGCTATTTACAATCCAGTA
AAAGACAATGGAGATGGTAATATGATATACCTTGTAAGCACTCTAGCAAA
CACTTGGGACCAGCCTCCAAAAGACAGTGCTATTTTAATACAAGGAGTAC
CCATATGGCTAGGCTTATTTGGATATTTAGACTACTGTAGACAAATTAAA
GCTGACAAAACATGGCTAGACAGTCATGTACTAGTAATTCAAAGTCCTGC
TATTTTTACTTACCCAAATCCAGGAGCAGGCAAATGGTATTGTCCACTAT
CACAAAGTTTTATAAATGGCAATGGTCCGTTTAATCAACCACCTACACTG
CTACAAAAAGCAAAGTGGTTTCCACAAATACAATACCAACAAGAAATTAT
TAATAGCTTTGTAGAATCAGGACCATTTGTTCCCAAATATGCAAATCAAA
CTGAAAGCAACTGGGAACTAAAATATAAATATGTTTTTACATTTAAGTGG
GGTGGACCACAATTCCATGAACCAGAAATTGCTGACCCTAGCAAACAAGA
GCAGTATGATGTCCCCGATACTTTCTACCAAACAATACAAATTGAAGATC
CAGAAGGACAAGACCCCAGATCTCTCATCCATGATTGGGACTACAGACGA
GGCTTTATTAAAGAAAGATCTCTTAAAAGAATGTCAACTTACTTCTCAAC
TCATACAGATCAGCAAGCAACTTCAGAGGAAGACATTCCCAAAAAGAAAA
AGAGAATTGGACCCCAACTCACAGTCCCACAACAAAAAGAAGAGGAGACA
CTGTCATGTCTCCTCTCTCTCTGCAAAAAAGATACCTTCCAAGAAACAGA
GACACAAGAAGACCTCCAGCAGCTCATCAAGCAGCAGCAGGAGCAGCAGC
TCCTCCTCAAGAGAAACATCCTCCAGCTCATCCACAAACTAAAAGAGAAT
CAACAAATGCTTCAGCTTCACACAGGCATGTTACCTTAACCAGATTTAAA
CCTGGATTTGAAGAGCAAACAGAGAGAGAATTAGCAATTATATTTCATAG
GCCCCCTAGAACCTACAAAGAGGACCTTCCATTCTATCCCTGGCTACCAC
CTGCACCCCTTGTACAATTTAACCTTAACTTCAAAGGCTAGGCCAACAAT
GTACACTTAGTAAAGCATGTTTATTAAAGCACAACCCCCAAAATAAATGT
AAAAATAAAAAAAAAAAAAAAAAAATAAAAAATTGCAAAAATTCGGCGCT
CGCGCGCATGTGCGCCTCTGGCGCAAATCACGCAACGCTCGCGCGCCCGC
GTATGTCTCTTTACCACGCACCTAGATTGGGGTGCGCGCGCTAGCGCGCG
CACCCCAATGCGCCCCGCCCTCGTTCCGACCCGCTTGCGCGGGTCGGACC
ACTTCGGGCTCGGGGGGGCGCGCCTGCGGCGCTTTTTTACTAAACAGACT
CCGAGCCGCCATTTGGCCCCCTAAGCTCCGCCCCCCTCATGAATATTCAT
AAAGGAAACCACATAATTAGAATTGCCGACCACAAACTGCCATATGCTAA
TTAGTTCCCCTTTTACAAAGTAAAAGGGGAAGTGAACATAGCCCCACACC
CGCAGGGGCAAGGCCCCGCACCCCTACGTCACTAACCACGCCCCCGCCGC
CATCTTGGGTGCGGCAGGGGGGGGC (SEQ ID NO: 886)
Annotations:

| Putative Domain | Base range |
|---|---|
| TATA Box | 87-93 |
| Cap Site | 110-117 |
| Transcriptional Start Site | 117 |
| 5′ UTR Conserved Domain | 185-254 |
| ORF2 | 286-660 |
| ORF2/2 | 286-656; 1998-2442 |
| ORF2/3 | 286-656; 2209-2641 |
| TAIP | 385-484 |
| ORF1 | 501-2489 |
| ORF1/1 | 501-656; 1998-2489 |
| ORF1/2 | 501-656; 2209-2442 |
| Three open-reading frame region | 2209-2439 |
| Poly(A) Signal | 2672-2678 |
| GC-rich region | 3076-3176 |

TABLE A17
Exemplary Anellovirus amino acid sequences (Gammatorquevirus)
Ring 4 (Gammatorquevirus)

| | |
|---|---|
| ORF2 | MVSISSSDFFKKTKFNEETQNQVWMSQIADSHDNICSCWHPFAHLLASIFPP |
| | GHKDRDLTINQILLR |
| | DYKEKCHSGGEEGENSGPTTGLITPKEEDIEKDGPEGAAEEDHTDALFAAAV |
| | ENFER (SEQ ID NO: 887) |
| ORF2/2 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFESGVDHNSMNQKLLTLANKSSMMSPILSTKQYKL |
| | KIQKDKTPDLSSMIGTTDEALLKKDLLKECQLTSQLIQISKQLQRKTFPKRKR |
| | ELDPNSQSHNKKKRRHCHVSSLSAKKIPSKKQRHKKTSSSSSSSSRSSSSSSR |
| | ETSSSSSTN (SEQ ID NO: 888) |
| ORF2/3 | MVSISSSDFFKKTKFNEETQNQVWMSQIADSHDNICSCWHPFAHLLASIFPP |
| | GHKDRDLTINQILLRDYKEKCHSGGEEGENSGPTTGLITPKEEDIEKDGPEGA |
| | AEEDHTDALFAAAVENFERSASNFRGRHSQKEKENWTPTHSPTTKRRGDTV |
| | MSPLSLQKRYLPRNRDTRRPPAAHQAAAGAAAPPQEKHPPAHPQTKRESTN |
| | ASASHRHVTLTRFKPGFEEQTERELAIIFHRPPRTYKEDLPFYPWLPPAPLVQ |
| | FNLNFKG (SEQ ID NO: 889) |
| TAIP | MRRRRTKYGCLKLLTLMIISAVAGIHLLTFLLPYFLLATKIVILLLTKFF (SEQ |
| | ID NO: 890) |
| ORF1 | MPFWWRRRRKFWTNNRFNYTKRRRYRKRWPRRRRRRRPYRRPVRRRRRK |
| | LRKVKRKKKSLIVRQWQPDSIRTCKIIGQSAIVVGAEGKQMYCYTVNKLINV |
| | PPKTPYGGGFGVDQYTLKYLYEEYRFAQNIWTQSNVLKDLCRYINVKLIFY |
| | RDNKTDFVLSYDRNPPFQLTKFTYPGAHPQQIMLQKHHKFILSQMTKPNGR |
| | LTKKLKIKPPKQMLSKWFFSKQFCKYPLLSLKASALDLRHSYLGCCNENPQ |
| | VFFYYLNHGYYTITNWGAQSSTAYRPNSKVTDTTYYRYKNDRKNINIKSHE |
| | YEKSISYENGYFQSSFLQTQCIYTSERGEACIAEKPLGIAIYNPVKDNGDGNM |
| | IYLVSTLANTWDQPPKDSAILIQGVPIWLGLFGYLDYCRQIKADKTWLDSHV |
| | LVIQSPAIFTYPNPGAGKWYCPLSQSFINGNGPFNQPPTLLQKAKWFPQIQYQ |
| | QEIINSFVESGPFVPKYANQTESNWELKYKYVFTFKWGGPQFHEPEIADPSK |
| | QEQYDVPDTFYQTIQIEDPEGQDPRSLIHDWDYRRGFIKERSLKRMSTYFST |
| | HTDQQATSEEDIPKKKKRIGPQLTVPQQKEEETLSCLLSLCKKDTFQETETQE |
| | DLQQLIKQQQEQQLLLKRNILQLIHKLKENQQMLQLHTGMLP (SEQ ID NO: |
| | 891) |
| ORF1/1 | MPFWWRRRRKFWTNNRFNYTKRRRYRKRWPRRRRRRRPYRRPVRRRRRK |
| | LRKWGGPQFHEPEIADPSKQEQYDVPDTFYQTIQIEDPEGQDPRSLIHDWDY |
| | RRGFIKERSLKRMSTYFSTHTDQQATSEEDIPKKKKRIGPQLTVPQQKEEETL |
| | SCLLSLCKKDTFQETETQEDLQQLIKQQQEQQLLLKRNILQLIHKLKENQQM |
| | LQLHTGMLP (SEQ ID NO: 892) |
| ORF1/2 | MPFWWRRRRKFWTNNRFNYTKRRRYRKRWPRRRRRRRPYRRPVRRRRRK |
| | LRKISKQLQRKTFPKRKR |
| | ELDPNSQSHNKKKRRHCHVSSLSAKKIPSKKQRHKKTSSSSSSSSRSSSSSSR |
| | ETSSSSSTN (SEQ ID NO: 893) |

TABLE N18
Exemplary Anellovirus nucleic acid sequence (Alphatorquevirus)-
Clade 1

| | |
|---|---|
| Name | Ring5.2 |
| Genus/Clade | Alphaatorquevirus Clade 1 |
| Accession Number | |

Full Sequence: 3696 bp
1 10 20 30 40 50
| | | | | |
ATTTTGTTCAGCCCGCCAATTTCTCTTTCAAACAGGCCAATCAGCTACTA
CTTCGTGCACTTCCTGGGGCGTGTCCTGCCGCTCTATATAAGCAGAGGCG
GTGACGAATGGTAGAGTTTTTCTTGGCCCGTCCGCGGCGAGAGCGCGAGC
GAAGCGAGCGATCGAGCGTCCCGAGGGCGGGTGCCGGAGGTGAGTTTACA
CACCGCAGTCAAGGGGCAATTCGGGCTCGGGACTGGCCGGGCTATGGGCA
AGATTCTTAAAAAATTCCCCCGATCCCTTTGCCGCCAGGACATAAAAACA
TGCCGTGGAGACCGCCGGTCCATAGTGTCCAGGGGCGAGAGGATCAGTGG
TTCGCAAGCTTTTTTCACGGCCACGATTCGTTTTGCGGCTGCGGTGACCC
TCTTGGCCATATTAATAGCATTGCTCATCGCTTTCCTCGCGCCGGTCCAC
CAAGGCCCCCTCCGGGGCTAGATCAGCCTAACCCCCGGGAGCAGGGCCCG
GCCGGACCCGGAGGGCCGCCCGCCATCTTGGCCCTGCCGGCTCCGCCCGC
GGAGCCTGACGACCCGCAGCCACGGCGTGGTGGTGGGGACGGTGGCGCCG
CCGCTGGCGCCGCAGACGACCATACACAACGAGACTACGACGAAGAAGAG
CTAGACGAGCTTTTCCGCGCCGCCGCCGAAGACGATTTGTAAGTAGGAGA
TGGCGCCGGCCTTACAGGCGCAGGAGGAGACGCGGGCGACGCAGACGCAG
ACGCAGACGCAGACATAAGCCCACCCTAATACTCAGACAGTGGCAACCTG
ACTGTATCAGACACTGTAAAATAACAGGATGGATGCCCCTCATTATCTGT
GGAAAGGGGTCCACCCAGTTCAACTACATCACCCACGCGGACGATATCAC
CCCCAGGGGAGCCTCCTACGGAGGCAATTTCACAAACATGACTTTCTCCC
TGGAGGCCATATATGAACAGTTCCTATACCACAGAAACAGGTGGTCGGCC
TCTAACCACGACCTAGAACTGTGCAGATACAAGGGGACCACCTTAAAACT
CTACAGACACCCAGAAGTAGACTACATAGTTACCTACAGCAGAACAGGAC
CCTTTGAAATCAGCCACATGACCTACCTCAGCACTCACCCCATGCTAATG
CTGCTAAACAAGCACCACATTGTGGTGCCCAGCTTAAAGACTAAGCCCAG
AGGCAGAAAGGCCATAAAAGTCAGGATAAGGCCCCCAAAACTCATGAACA
ACAAGTGGTACTTCACCAGAGACTTCTGTAACATAGGCCTCTTCCAGCTC
TGGGCCACAGGCTTAGAACTCAGAAACCCCTGGCTCAGAATGAGCACCCT
GAGCCCCTGCATAGGCTTTAATGTCCTCAAAAACAGCATTTACACAAACC
TCAGCAACCTGCCACAATACAAAAACGAAAGACTAAACATCATTAACAAC
ATACTTCACCCACAAGAAATTACAGGTACAAACAACAAAAAGTGGCAGTA
CACATACACCAAACTCATGGCCCCTATTTACTATTCAGCAAACAGGGCCA
GCACCTATGACTGGGAAAATTACAGCAAAGAAACAAACTACAATAATACA
TATGTTAAATTTACCCAGAAAAGACAGGAAAAACTAACTAAAATTAGAAA
AGAGTGGCAGATGCTTTATCCACAACAACCCACAGCACTGCCAGACTCCT
ATGACCTCCTACAAGAGTATGGCCTCTACAGTCCATACTACCTAAACCCC
ACAAGAATAAACCTAGACTGGATGACCCCATACACACACGTCAGATACAA
TCCCCTAGTAGACAAGGGCTTTGGAAACAGAATATACATCCAGTGGTGCT
CAGAAGCAGATGTTAGCTACAACAGGACAAAATCCAAGTGTCTGCTACAA
GACATGCCCCTGTTTTTCATGTGCTATGGCTACATAGACTGGGCAATAAA
AAACACTGGAGTGTCATCTCTAGTGAAGGACGCCAGAATCTGCATCAGGT
GTCCCTACACAGAGCCACAACTAGTTGGCTCCACAGAAGACATAGGCTTT
GTACCCATCTCAGAAACCTTCATGAGGGGCGACATGCCGGTACTTGCACC
ATACATACCGTTAAGCTGGTTTTGCAAGTGGTATCCCAACATAGCTCACC
AAAAGGAAGTCCTTGAGTCAATCATTTCCTGCAGCCCCTTCATGCCCCGT
GACCAAGACATGAACGGTTGGGATATCACAATCGGTTACAAAATGGACTT
CTTATGGGGCGGTTCCCCTCTCCCCTCACAGCCAATCGACGACCCCTGCC
AGCAGGGAACCCACCCGATTCCCGACCCCGATAAACACCCTCGCCTCCTA
CAAGTCTCGAACCCGAAACTACTCGGACCGAGGACAGTGTTCCACAAGTG
GGACATCAGACGTGGGCAGTTTAGCAAAAGAAGTATTAAGAGAGTGTCAG
AATACTCAAGCGATGATGAATCTCTTGCGCCAGGTCTCCCATCAAAGCGA
AACAAGCTCGACTCGGCGTTCCGAGGAGAAAATCGAGAGCAAAAAGAATG
CTATTCTCTCCTCAAAGCGCTCGAGGAAGAAGAGACCCCAGAAGAAGAAG
AACCAGCACCCCAAGAAAAAGCCCAGAAAGAGGAGCTACTCCACCAGCTC
CAGCTCCAGAGACGCCACCAGCGAGTCCTCAGACGAGGGCTCAAGCTCGT
CTTTACAGACATCCTCCGACTCCGCCAGGGAGTCCACTGGAACCCGGAGC
TCACATAGCGCCCCCACCTTACATACCAGACCTGCTTTTTCCCAATACTG
GTAAAAAAAAAAAATTCTCTCCCTTCGATTGGGAGACAGAGGCGCAAATA
GCGGGGTGGATGCGGCGGCCCATGCGCTTCTATCCCTCAGACACCCCTCA
CTACCCGTGGCTACCCCCCGAGCGAGATATCCCGAAAATATGTAACATAA
ACTTCAAAATAAAGCTTCAAGAGTGAGTGATTCGAGGCCCTCCTCTGTTC
ACTTAGCGGTGTCTACCTCTTAAGGTCACTAAGCACTCCGAGCGTAAGCG
AGGAGTGCGACCCTCTACCAAGGGGCAACTTCCTCGGGGTCCGGCGCTAC
GCGCTTCGCGCTGCGCCGGACATCTCGGACCCCTCGACCCGAATCGCTTG
CGCGATTCGGACCTGCGGCCTCGGGGGGGTCGGGGGCTTTACTAAACAGA
CTCCGAGGTGCCATTGGACACTGTAGGGGGTGAACAGCAACGAAAGTGAG
TGGGGCCAGACTTCGCCATAAGGCCTTTATCTTCTTGCCATTGGATAGTG
ACTTCCGGGTCCGCCTGGGGGCCGCCATTTTAGCTTCGGCCGCCATTTTA
GGCCCTCGCGGGCCTCCGTAGGCGCGCTTTAGTGACGTCACGGCAGCCAT
TTTGTCGTGACGTTTGAGACACGTGATGGGGGCGTGCCTAAACCCGGAAG
CATCCCTGGTCACGTGACTCTGACGTCACGGCGGCCATCTTGTGCTGTCC
GCCATCTTGTAACTTCCTTCCGCTTTTTCAAAAAAAAAGAGGAAGTGTGA
CGTAGCGGCGGGGGGGCGGCGCGCTTCGCGCGCCGCCCACCAGGGGGCGC
TGCGCGCCCCCCGCGCATGCGCAGGGGCCTCTCGAGGGGCTCCGCCCCCC
CCCCGTGCTAAATTTACCGCGCATGCGCGACCACGCCCCCGCCGCC (SEQ ID NO: 894)
Annotations:

| Putative Domain | Base range |
|---|---|
| TATA Box | 85-91 |
| Cap Site | 108-115 |
| Transcriptional Start Site | 115 |
| 5′ UTR Conserved Domain | 178-248 |
| ORF2 | 300-692 |
| ORF2/2 | 300-688; 2282-2804 |
| ORF2/3 | 300-688; 2484-2976 |
| ORF2t/3 | 300-349: 2484-2976 |
| TAIP | 322-471 |
| ORF1 | 572 -2758 |
| ORF1/1 | 572-688; 2282-2758 |
| ORF1/2 | 572-688; 2484-2804 |
| Three open-reading frame region | 2484-2755 |
| Poly(A) Signal | 3018-3023 |
| GC-rich region | 3555-3696 |

TABLE A18
Exemplary Anellovirus amino acid sequences (Alphatorquevirus) Clade 1
Ring 5.2 (Alphaatorquevirus) Clade 1

| | |
|---|---|
| ORF2 | MPWRPPVHSVQGREDQWFASFFHGHDSFCGCGDPLGHINSIAHRFPRAGPP |
| | RPPPGLDQPNPREQGPAGPGGPPAILALPAPPAEPDDPQPRRGGGDGGAAAG |
| | AADDHTQRDYDEEELDELFRAAAEDDL (SEQ ID NO: 895) |
| ORF2/2 | MPWRPPVHSVQGREDQWFASFFHGHDSFCGCGDPLGHINSIAHRFPRAGPP |
| | RPPPGLDQPNPREQGPAGPGGPPAILALPAPPAEPDDPQPRRGGGDGGAAAG |
| | AADDHTQRDYDEEELDELFRAAAEDDFQSTTPASREPTRFPTPINTLASYKS |
| | RTRNYSDRGQCSTSGTSDVGSLAKEVLRECQNTQAMMNLLRQVSHQSETSS |
| | TRRSEEKIESKKNAILSSKRSRKKRPQKKKNQHPKKKPRKRSYSTSSSSRDAT |
| | SESSDEGSSSSLQTSSDSARESTGTRSSHSAPTLHTRPAFSQYW (SEQ ID NO: |
| | 896) |
| ORF2/3 | MPWRPPVHSVQGREDQWFASFFHGHDSFCGCGDPLGHINSIAHRFPRAGPP |
| | RPPPGLDQPNPREQGPAGPGGPPAILALPAPPAEPDDPQPRRGGGDGGAAAG |
| | AADDHTQRDYDEEELDELFRAAAEDDLSPIKAKQARLGVPRRKSRAKRMLF |
| | SPQSARGRRDPRRRRTSTPRKSPERGATPPAPAPETPPASPQTRAQARLYRHP |
| | PTPPGSPLEPGAHIAPPPYIPDLLFPNTGKKKKFSPFDWETEAQIAGWMRRPM |
| | RFYPSDTPHYPWLPPERDIPKICNINFKIKLQ (SEQ ID NO: 897) |
| ORF2t/3 | MPWRPPVHSVQGREDQWSPIKAKQARLGVPRRKSRAKRMLFSPQSARGRR |
| | DPRRRRTSTPRKSPERGATPPAPAPETPPASPQTRAQARLYRHPPTPPGSPLEP |
| | GAHIAPPPYIPDLLFPNTGKKKKFSPFDWETEAQIAGWMRRPMRFYPSDTPH |
| | YPWLPPERDIPKICNINFKIKLQE (SEQ ID NO: 898) |
| TAIP | IVSRGERISGSQAFFTATIRFAAAVTLLAILIALLIAFLAPVHQGPLRG (SEQ ID |
| | NO: 899) |
| ORF1 | TAWWWGRWRRRWRRRRPYTTRLRRRRARRAFPRRRRRRFVSRRWRRPYR |
| | RRRRRGRRRRRRRRRHKPTLILRQWQPDCIRHCKITGWMPLIICGKGSTQFN |
| | YITHADDITPRGASYGGNFTNMTFSLEAIYEQFLYHRNRWSASNHDLELCRY |
| | KGTTLKLYRHPEVDYIVTYSRTGPFEISHMTYLSTHPMLMLLNKHHIVVPSL |
| | KTKPRGRKAIKVRIRPPKLMNNKWYFTRDFCNIGLFQLWATGLELRNPWLR |
| | MSTLSPCIGFNVLKNSIYTNLSNLPQYKNERLNIINNILHPQEITGTNNKKWQ |
| | YTYTKLMAPIYYSANRASTYDWENYSKETNYNNTYVKFTQKRQEKLTKIR |
| | KEWQMLYPQQPTALPDSYDLLQEYGLYSPYYLNPTRINLDWMTPYTHVRY |
| | NPLVDKGFGNRIYIQWCSEADVSYNRTKSKCLLQDMPLFFMCYGYIDWAIK |
| | NTGVSSLVKDARICIRCPYTEPQLVGSTEDIGFVPISETFMRGDMPVLAPYIPL |
| | SWFCKWYPNIAHQKEVLESIISCSPFMPRDQDMNGWDITIGYKMDFLWGGS |
| | PLPSQPIDDPCQQGTHPIPDPDKHPRLLQVSNPKLLGPRTVFHKWDIRRGQFS |
| | KRSIKRVSEYSSDDESLAPGLPSKRNKLDSAFRGENREQKECYSLLKALEEE |
| | ETPEEEEPAPQEKAQKEELLHQLQLQRRHQRVLRRGLKLVFTDILRLRQGVH |
| | WNPELT (SEQ ID NO: 900) |
| ORF1/1 | TAWWWGRWRRRWRRRRPYTTRLRRRRARRAFPRRRRRRFPIDDPCQQGT |
| | HPIPDPDKHPRLLQVSNPKLLGPRTVFHKWDIRRGQFSKRSIKRVSEYSSDDE |
| | SLAPGLPSKRNKLDSAFRGENREQKECYSLLKALEEEETPEEEEPAPQEKAQ |
| | KEELLHQLQLQRRHQRVLRRGLKLVFTDILRLRQGVHWNPELT (SEQ ID |
| | NO: 901) |
| ORF1/2 | TAWWWGRWRRRWRRRRPYTTRLRRRRARRAFPRRRRRRFVSHQSETSSTR |
| | RSEEKIESKKNAILSSK |
| | RSRKKRPQKKKNQHPKKKPRKRSYSTSSSSRDATSESSDEGSSSSLQTSSDSA |
| | RESTGTRSSHSAPTLHTRPAFSQYW (SEQ ID NO: 902) |

TABLE N19
Exemplary Anellovirus nucleic acid sequence (Alphatorquevirus)-
Clade 3

| | |
|---|---|
| Name | Ring 6.0 |
| Genus/Clade | Alphatorquevirus-Clade 3 |
| Accession Number | |

Full Sequence: 3828 bp
1 10 20 30 40 50
| | | | | |
GTGCTACGTCACTAACCTACGTGTCCGTCTCCCATAGGCCGGACACCGTA
TACGTCATACACTTCCTGGGCATGGTCTACGTGATAATATAAGTGGCTGC
ACTTCCGAATGGCTGAGTTTTCCACGCCCGTCCGCAGCGAGGACGCCACG
GAGGGGGATCCGCGCGTCCCGAGGGCGGGTGCCGGAGGTGAGTTTACACA
CCGCAGTCAAGGGGCAATTCGGGCTCGGGACTGGCCGGGCTATGGGCAAG
GCTCTTAAAAATGCACTTTTCTAGGTGCAGTAGAAAGAAAAGGACATTGT
CACTGCTACCACTGTACCATTCACAGAAAGCTAGGCCATCTGTGACAGGT
ATGTGGAGACCCCCGACTCGAAATGCGTTCAATATTCAACGTGACTGGTT
CTACAGTTGCTTTCACTCCCACGCTTCTATGTGCGGCTGTGCTGATTTTA
TTGGTCATTTCAATCATATCGCTGCTATGCTCGGCCGTCCGGAAGACCAG
AACCCTCCTCCGCCACCCGGGGCTCTGAGACCCCTACCCGCTCTCCCGGC
CTCTTCCGAGGCACCCGGTGATCGAGCGCCATGGCCTATGGGTGGTGGCG
GAGGCGACGGAGGCGCCCGTGGTGGAGGAGGAGATGGCGCCGCTGGAGAC
GCCGTCGGAGACCCCGCAGACGCCGACCTCGTCGCCGCTATCGACGCCGC
AGAACAGTAAGGAGGCGCGGCAGGGGGAGGTGGACTAGAGCACACAGGAG
ATGGCGCCGCAAGGGAAAACGCAGTCGCAAAAAAAAGATTATTATAAGAC
AATGGCAGCCCAACTACACTCGCAGATGCAACATAGTGGGCTACATGCCT
CTACTAATATGTGGGGAAAATACTGTTGCTACAAACTATGCCACCCACTC
AGACGACAGCTACTACCCCGGACCCTTTGGGGGGGGAATGACTACAGACA
AATTTACTCTAAGAATACTGTATGATGAGTACAAAAGGTTCATGAACTAC
TGGACCTCTTCAAACGAGGACCTAGACCTATGTAGATACCTGGGATGCAC
TCTATATGTGTTTAGACACCCAGAAGTAGACTTTATAATCATTATAAATA
CCTCTCCTCCATTCCTAGACACAGAAATAACAGGGCCTAGCATACACCCA
GGTATGATGGCCCTTAACAAAAGAAGCAGATGGATACCTAGCATAAAAAA
CAGACCAGGCAGAAAGCACTATATAAAGATTAAAGTAGGAGCCCCCCGAA
TGTTCACAGATAAGTGGTACCCCCAAACAGACCTCTGTGACATGACACTC
CTAACGATCTTTGCCAGTGCGGCGGATATGCAATATCCGTTCGGCTCACC
ACTAACTGACACCATAGTTGTGTCATTCCAAGTTCTGCAATCCATGTACA
ACGACTGCCTGAGTGTACTTCCTGATAATTTTGCAGAGACATCAGGCAAA
GGCACCCAACTACATGAGAACATAATACAACATCTGCCCTACTACAACAC
CACACAAACACAAGCACAATTTAAAAGATTTATAGAAAACATGAATGCAA
CAAATGGAGACAATATATGGGCAAGCTACATAAACACAACCAAGTTCTCA
TCCGCAAACACTCCAAAGAATGACACAGGCATAGGAGGCCCTTACACTAC
ATATTCAGACTCATGGTACAAAGGCACAGTATACAATGACAAAATTAAAA
CCATACCAATAAAAGCAAGCAAGTTATACTACGAGCAAACCAAAAACCTC
ATTGGCATTACATTCACTGGATCCACACACAGACTCCATTACTGTGGAGG
CCTATACTCCTCCGTATGGCTATCAGCAGGTAGATCCTACTTTGAAACCA
AAGGCCCATACACAGACATAACTTACAACCCCTTTTCAGACAGAGGAGAG
GGTAACATGCTATGGATAGACTGGCTAACTAAAAATGACTCAGTGTACTC
AAAAACAAGTAGCAAGTGTCTTATAGAAAACCTGCCCCTGTGGGCCTCAG
TATACGGATATAAAGAATACTGCAGCAAGGTAACAGGAGACACAAACATA
GAACACAACTGTAGATGTGTTATCAGAAGCCCCTACACAGTACCACAACT
GTTAGACCACAACAATCCCTTCAGAGGATACGTGCCTTATAGCTTCAACT
TTGGAAATGGTAAAATGCCAGGCGGTAGCAGCCTAGTGCCCATTAGAATG
AGAGCCAAGTGGTACCCCACTCTGTTCCACCAAAAAGAAGTTCTAGAAGC
CATAGCACAGGCGGGCCCCTTCGCATACCACTCAGATATTAAAAAAGTGT
CCCTGGGCATAAAGTACAGATTTAAGTGGGTGTGGGGTGGCAACCCCGTG
TCCCAACAGGTTGTTAGAAACCCCTGCAAGACCACCCAAGGTTCCTCGGG
CAATAGAGTGCCTCGATCAATACAAGTCGTTGACCCGCGGTACAACACGC
CAGAACTCACCATACACGCGTGGGACTTCAGACATGGGTTCTTTGGCAGA
AAAGCTATTAAGAGAATGCAAGAACAACCAATACCTCATGACACTTTTTC
AGCAGGGTTCAAGCGCAGTCGCCGAGATACAGAAGCACTCCAATGCAGCC
AAGAAGAGCAACAAAAAGAAAACTTACTTTTCCCAGTCCAGCAGCTCAAG
CGAGTCCCCCCGTGGGAGACCTCGCAAGAGAGCCAAAGCGAGGAAGAAAA
CTCGCAAAAACAGGAGACCCTCTCCCAGCAACTCAGAGACCAGCTGCACA
AGCAGCGGCTCATGGGAGAGCAACTCCGATCGCTCCTCTACCAAATGCAG
AGGGTCCAACAAAATCAACACATAAACCCTATGTTATTGCCAAAGGGTCT
GGCATTAACTTCTATTTCTCACAATGTAATATAGATATGTTTGGTGACCC
CAAACCCTACAAGCCCTCCTCCAATGACTGGAAGGAGGAGTACGAGGCCG
CAAAGTACTGGGACAGACCCCCCAGACGCGACCTGAGGAGCACCCCCTTC
TACCCCTGGGCCCCCACCCCCAAACCATACAATGTCAACTTTGCCCTCAA
CTACAAATAAACGGTGGCCGTGGGAGTTTCACTTGTCGGTGTCTACCTCT
TAAGGTCACTAAGCACTCCGAGCGTAAGCGAGGAGTGCGACCCTTCACCA
AGGGCAACTCCCTCGAAGTCCGGCGCTACGCGCTTCGCGCTGCGCCGGAC
ATCTCGGACCCCCCCTCGACCCGAATCGCTTGCGCGATTCGGACCTGCGG
CCTCGGGGGGGTCGGGGGCTTTACTAAACAGACTCCGAGGTGCCATTGGA
CACTGAGGGGGTGAACAGCAACGAAAGTGAGTGGGGCCAGACTTCGCCAT
AAGGCCTTTATCTTCTTGCCATTTGTCCGCGACCGGGGGTCGCTCCTAGG
CGCGGACCCCGTTTCGGGGTCCTTCCGGGTTCATCGGCGCCGTTCCAGTG
ACGTCACGGGCGCCATGTTAAGTGGCTGTCGCCGAGGATTGACGTCACAG
TTCAAAGGTCATCCTCGGCGGTAACCGCAAACATGGCGGTCAATCTCTTC
CGGGTCAAAGGTCGTGCATACGTCATAAGTCACATGACAGGGGTCCACTT
AAACACGGAAGTAGGCCCCGACATGTGACTCGTCACGTGTGTACACGTCA
CGGCCGCCATTTTGTTTTACAAAATGGCCGACTTCCTTCCTGTTTTTTAA
AAAAAGGCGCGAAAAAACCGTCGGCGGGGGCCGCGCGCTGCGCGCGCGGG
AGGCAATGCCTCCCCCCCCCCGCGCGCATGCGCGCGGGTCCCCCCCCCTC
CGGGGGGCTCCGCCCCCCGGCCCCCCCC (SEQ ID NO: 903)
Annotations:

| Putative Domain | Base range |
|---|---|
| TATA Box | 85-92 |
| Cap Site | 109-116 |
| Transcriptional Start Site | 116 |
| 5′ UTR Conserved Domain | 176-246 |
| ORF2 | 351-710 |
| ORF2/2 | 351-706; 2360-2825 |
| ORF2/3 | 351-706; 2556-3060 |
| TAIP | 373-528 |
| ORF1 | 581-2884 |
| ORF1/1 | 581-706; 2360-2884 |
| ORF1/2 | 581-706; 2556-2825 |
| Three open-reading frame region | 2556-2821 |
| Poly(A) Signal | 3055-3061 |
| GC-rich region | 3720-3828 |

TABLE A19
Exemplary Anellovirus amino acid sequences (Alphatorquevirus)-Clade 3
Ring 6.0 (Alphatorquevirus)

| | |
|---|---|
| ORF2 | MWRPPTRNAFNIQRDWFYSCFHSHASMCGCADFIGHFNHIAAMLGRPEDQ |
| | NPPPPPGALRPLPALPASSEAPGDRAPWPMGGGGGDGGARGGGGDGAAGD |
| | AVGDPADADLVAAIDAAEQ (SEQ ID NO: 904) |
| ORF2/2 | MWRPPTRNAFNIQRDWFYSCFHSHASMCGCADFIGHFNHIAAMLGRPEDQ |
| | NPPPPPGALRPLPALPASSEAPGDRAPWPMGGGGGDGGARGGGGDGAAGD |
| | AVGDPADADLVAAIDAAEQLLETPARPPKVPRAIECLDQYKSLTRGTTRQNS |
| | PYTRGTSDMGSLAEKLLRECKNNQYLMTLFQQGSSAVAEIQKHSNAAKKS |
| | NKKKTYFSQSSSSSESPRGRPRKRAKARKKTRKNRRPSPSNSETSCTSSGSW |
| | ESNSDRSSTKCRGSNKINT (SEQ ID NO: 905) |
| ORF2/3 | MWRPPTRNAFNIQRDWFYSCFHSHASMCGCADFIGHFNHIAAMLGRPEDQ |
| | NPPPPPGALRPLPALPASSEAPGDRAPWPMGGGGGDGGARGGGGDGAAGD |
| | AVGDPADADLVAAIDAAEQVQAQSPRYRSTPMQPRRATKRKLTFPSPAAQA |
| | SPPVGDLAREPKRGRKLAKTGDPLPATQRPAAQAAAHGRATPIAPLPNAEG |
| | PTKSTHKPYVIAKGSGINFYFSQCNIDMFGDPKPYKPSSNDWKEEYEAAKY |
| | WDRPPRRDLRSTPFYPWAPTPKPYNVNFALNYK (SEQ ID NO: 906) |
| TAIP | MRSIFNVTGSTVAFTPTLLCAAVLILLVISIISLLCSAVRKTRTLLRHPGL (SEQ |
| | ID NO: 907) |
| ORF1 | MAYGWWRRRRRRPWWRRRWRRWRRRRRPRRRRPRRRYRRRRTVRRRGR |
| | GRWTRAHRRWRRKGKRSRKKKIIIRQWQPNYTRRCNIVGYMPLLICGENTV |
| | ATNYATHSDDSYYPGPFGGGMTTDKFTLRILYDEYKRFMNYWTSSNEDLD |
| | LCRYLGCTLYVFRHPEVDFIIIINTSPPFLDTEITGPSIHPGMMALNKRSRWIPS |
| | IKNRPGRKHYIKIKVGAPRMFTDKWYPQTDLCDMTLLTIFASAADMQYPFG |
| | SPLTDTIVVSFQVLQSMYNDCLSVLPDNFAETSGKGTQLHENIIQHLPYYNTT |
| | QTQAQFKRFIENMNATNGDNIWASYINTTKFSSANTPKNDTGIGGPYTTYSD |
| | SWYKGTVYNDKIKTIPIKASKLYYEQTKNLIGITFTGSTHRLHYCGGLYSSV |
| | WLSAGRSYFETKGPYTDITYNPFSDRGEGNMLWIDWLTKNDSVYSKTSSKC |
| | LIENLPLWASVYGYKEYCSKVTGDTNIEHNCRCVIRSPYTVPQLLDHNNPFR |
| | GYVPYSFNFGNGKMPGGSSLVPIRMRAKWYPTLFHQKEVLEAIAQAGPFAY |
| | HSDIKKVSLGIKYRFKWVWGGNPVSQQVVRNPCKTTQGSSGNRVPRSIQVV |
| | DPRYNTPELTIHAWDFRHGFFGRKAIKRMQEQPIPHDTFSAGFKRSRRDTEA |
| | LQCSQEEQQKENLLFPVQQLKRVPPWETSQESQSEEENSQKQETLSQQLRD |
| | QLHKQRLMGEQLRSLLYQMQRVQQNQHINPMLLPKGLALTSISHNVI (SEQ |
| | ID NO: 908) |
| ORF1/1 | MAYGWWRRRRRRPWWRRRWRRWRRRRRPRRRRPRRRYRRRRTVVRNPC |
| | KTTQGSSGNRVPRSIQVVDPRYNTPELTIHAWDFRHGFFGRKAIKRMQEQPI |
| | PHDTFSAGFKRSRRDTEALQCSQEEQQKENLLFPVQQLKRVPPWETSQESQS |
| | EEENSQKQETLSQQLRDQLHKQRLMGEQLRSLLYQMQRVQQNQHINPMLL |
| | PKGLALTSISHNVI (SEQ ID NO: 909) |
| ORF1/2 | MAYGWWRRRRRRPWWRRRWRRWRRRRRPRRRRPRRRYRRRRTGSSAVA |
| | EIQKHSNAAKKSNKKKTYFSQSSSSSESPRGRPRKRAKARKKTRKNRRPSPS |
| | NSETSCTSSGSWESNSDRSSTKCRGSNKINT (SEQ ID NO: 910) |

TABLE N20
Exemplary Anellovirus nucleic acid sequence (Alphatorquevirus)-
Clade 7

| | |
|---|---|
| Name | Ring7 |
| Genus/Clade | Alphatorquevirus-Clade 7 |
| Accession Number | |

Full Sequence: 3815 bp
1 10 20 30 40 50
| | | | | |
AAGATCGTCACTAACCACGTGACTCCTCTCGCCCAATCAGTGTCTACGTC
GTCCATTTCCTGGGCATGGTCTACATCCTGATATAAAGCGATGCACTTCC
GAATGGCTGAGTTTTCCACGCCCGTCCGCGGCGAGATCGCGACGGAGGAG
CGATCGAGCGTCCCGAGGGCGGGTGCCGGAGGTGAGTTTACACACCGCAG
TCAAGGGGCAATTCGGGCTCGGGACTGGCCGGGCTATGGGCAAGGCTCTT
AAAGCGTACGTCCCCCGCTATGTTTCTCGGCAGGGTGTGGAGGAAACAGA
AAAGGAAAGTGCTTCTGCTGGCTGTGCGAGCTACACAGAAAACATCTTCC
ATGAGTATCTGGCGTCCCCCCCTTGGGAATGTCTCCTACAGGGAGAGAAA
TTGGCTTCAGGCCGTCGAAACATCCCACAGTTCTTTTTGTGGCTGTGGTG
ATTTTATTCTTCATCTTACTAATTTGGCTGCACGCTTTGCTCTCCAGGGG
CCCCCGCCAGAGGGTGGTCCACCTCGGCCGAGGCCGCCGCTCCTGAGAGC
GCTGCCGGCCCCCGAGGTCCGCAGGGAGACGCGCACAGAGAACCGGGGCG
CCTCCGGTGAGCCATGGCCTGGCGATGGTGGTGGCAGAGACGATGGCGCC
GCCGCCGGTGGCCCCGCAGACGGTGGAGACGCCTACGACGCCGGAGACCT
AGACGACCTGTTCGCCGCCGTCGAAGAAGAACAACAGTAAGGAGGCGGAG
GTGGAGGGGCAGACGTGGGCGACGCACATACACCCGACGCGCGGTCAGAC
GCAGACGCAGACCCAGAAAGAGACTTGTACTGACTCAGTGGAGCCCCCAG
ACAGTCAGAAACTGCTCAATAAGGGGCATAGTGCCCATGGTAATATGCGG
ACACACAAAAGCAGGTAGAAACTATGCTATTCATAGCGAGGACTTCACCA
CACAGATACAACCCTTCGGGGGCAGTTTCAGCACGACCACCTGGTCCCTA
AAAGTGCTGTGGGACGAGCACCAGAAATTCCAGAACAGATGGTCCTACCC
AAACACACAACTAGACCTGGCCAGATACAGAGGGGTCACCTTCTGGTTCT
ACAGAGACCAGAAAACAGACTATATAGTACAGTGGAGTAGGAATCCCCCT
TTTAAACTCAATAAATACAGCAGTGCCATGTACCACCCGGGCATGATGAT
GCAGGCCAAAAGGAAACTAGTTGTACCTAGTTTCCAGACCAGACCCAAAG
GCAAGAAGAGATACAGAGTCACAATAAAACCCCCTAACATGTTTGCTGAC
AAGTGGTACACTCAAGAGGACCTGTGTCCGGTACCTCTTGTGCAAATTGT
GGTTTCTGCGGCGAGCCTGCTACATCCGTTCTGCCCACCACAAACGAACA
ACCCTTGCATCACCTTCCAGGTTTTGAAAGACATATATGATGAATGCATA
GGAGTTAACGAAACTATGAAAGATAAGTATAAGAAATTACAAACAACACT
ATACACCACTTGCACATACTATCAAACAACACAAGTACTGGCACAGCTAT
CTCCTGCCTTTCAACCTGCTATGAAACCTACTACTACACAATCAGCAGCT
ACAGCGACAACACTAGGAAACTATGTACCAGAGTTAAAGTACAACAATGG
CTCTTTTCACACAGGACAAAACGCAGTATTCGGCATGTGCTCATACAAAC
CAACAGACAGCATAATGACAAAAGCTAATGGCTGGTTTTGGCAAAACCTA
ATGGTAGACAACAACCTACATAGTTCTTATGGCAAGGCAACATTAGAATG
CATGGAGTATCACACAGGCATATACAGCTCTATATTTCTAAGTCCACAAA
GATCTTTAGAATTCCCAGCAGCATACCAAGACGTTACATACAACCCTAAC
TGTGATAGAGCAGTTGGAAACGTAGTTTGGTTTCAGTACAGCACTAAAAT
GGATACAAATTTTGATGAAACAAAATGTAAATGTGTCCTTAAAAACATTC
CACTGTGGGCGGCCTTCAATGGCTACTCAGACTTTATAATGCAAGAACTC
AGCATAAGTACAGAAATCCACAACTTTGGCATAGTGTGCTTTCAGTGCCC
GTACACTTTTCCCCCCTGTTTCAATAAAAACAAACCCCTAAAGGGGTACG
TGTTCTATGACACCACCTTTGGTAATGGAAAAATGCCAGACGGATCGGGG
CACGTACCCATCTACTGGCAGCAGAGATGGTGGATCAGACTAGCCTTCCA
GGTCCAGGTCATGCATGACTTTGTACTAACAGGCCCCTTTAGCTACAAAG
ATGACCTAGCAAACACCACACTCACAGCCAGATACAAATTTAAATTCAAA
TGGGGCGGCAATATCATCCCTGAACAGATTATCAAGAACCCGTGTCACAG
AGAGCAGTCCCTCGCTTCCTATCCCGATAGACAACGTCGCGACCTACAAG
TTGTTGACCCATCAACCATGGGCCCGATCTACACCTTCCACACATGGGAC
TGGCGACGGGGGCTTTTTGGTGCAGATGCTATCCAGAGAGTGTCACAAAA
ACCGGGAGATGCTCTCCGCTTTACAAACCCTTTCAAGAGACCCAGATATC
TTCCCCCGACAGACAGAGAAGACTACCGACAAGAAGAAGACTTCGCTTTA
CAGGAAAAAAGACGGCGCACATCCACAGAAGAAGCCCAGGACGAGGAGAG
CCCCCCGGAAAGCGCGCCGCTCCTACAGCAGCAGCAGCAGCAGCGGCAGC
TCTCAGTCCACCTCGCGGAGCAGCAGCGACTCGGAGTCCAACTCCGATAC
ATCCTCCAAGAAGTCCTCAAAACGCAAGCGGGTCTCCACCTAAACCCCCT
ATTATTAGGCCCGCCACAAACAAGGTCTATCTCTTTGAGCCCTCCAAAGG
CCTACTCCCCATAGTAGGAAAAGAGGCCTGGGAGGACGAGTACTGCACCT
GCAAGTACTGGGATCGCCCTCCCAGAACCAACCACCTAGACATCCCCACT
TATCCCTGGATGCCCACAAACTTCAAAGTCAGCTTCAAACTTGGATTTAA
ACCCTAAATAAAAATACAAGGCCGTACACTGTTCACTTGTCGGTGTCTAC
CTCTATAAGTCACTAAGCACTCCGAGCGCAGCGAGGAGTGCGACCCTCAG
CGGTGGGTGCAACGCCCTCGGCGGCCGCGCGCTACGCCTTCGGCTGCGCG
CGGCACCTCGGACCCCCGCTCGTGCTGACACGCTCGCGCGTGTCAGACCA
CTTCGGGCTCGCGGGGGTCGGGAATTTTGCTAAACAGACTCCGAGTTGCT
CTTGGACACTGTAGCTGTGAATCAGTAACGAAAGTGAGTGGGGCCAGACT
TCGCCATAAGGCCTTTATCTTCTTGCCATTGGTCCGTCTCGGGGGTCGCC
ATAGGCTTCGGGCTCGGTTTTAGGCCTTCCGGACTACCAAAATGGCGGAT
TCCGTGACGTCATGGCCGCCATTTTAAGTAAGGCGGAACAGGCTGTCACC
CCGTGTCAAAGTTCAGGGGTCAGCCTTCCGCTTTACACAAAATGGAGGTC
AATATCTTCCGGGTCAAAGGTCGCTACCGCGTCATAAGTCACGTGGGGAA
GGCTGCTGTGAATCCGGAAGTAGCTGACCCACGTGACTTGTCACGTGACT
AGCACGTCACGGCAGCCATTTTGAATCACAAAATGGCCGACTTCCTTCCT
CTTTTTTAAAAATAACGGCCCGGCGGCGGCGCGCGCGCTTCGCGCCGCTC
CGCCCCCCCCGCGCATGCGCGGGACCCCCCCCCGCGGGGGGCTCCGCCCC
CCGGTCCCCCCCCCG (SEQ ID NO: 911)
Annotations:

| Putative Domain | Base range |
|---|---|
| TATA Box | 82-87 |
| Cap Site | 103-110 |
| Transcriptional Start Site | 110 |
| 5′ UTR Conserved Domain | 170-240 |
| ORF2 | 351-740 |
| ORF2/2 | 351-737; 2378-2843 |
| ORF2/3 | 351-737; 2526-3057 |
| TAIP | 379-543 |
| ORF1 | 614 -2911 |
| ORF1/1 | 614-737; 2378-2911 |
| ORF1/2 | 614-737; 2526-2843 |
| Three open-reading frame region | 2526-2840 |
| Poly(A) Signal | 3056-3062 |
| GC-rich region | 3716-3815 |

TABLE A20
Exemplary Anellovirus amino acid sequences (Alphatorquevirus)-Clade 7
Ring7.0 (Alphatorquevirus)

| | |
|---|---|
| ORF2 | MSIWRPPLGNVSYRERNWLQAVETSHSSFCGCGDFILHLTNLAARFALQGPP |
| | PEGGPPRPRPPLLRA |
| | LPAPEVRRETRTENRGASGEPWPGDGGGRDDGAAAGGPADGGDAYDAGD |
| | LDDLFAAVEEEQQ (SEQ ID NO: 912) |
| ORF2/2 | MSIWRPPLGNVSYRERNWLQAVETSHSSFCGCGDFILHLTNLAARFALQGPP |
| | PEGGPPRPRPPLLRA |
| | LPAPEVRRETRTENRGASGEPWPGDGGGRDDGAAAGGPADGGDAYDAGD |
| | LDDLFAAVEEEQQLSRTRVTESSPSLPIPIDNVATYKLLTHQPWARSTPSTHG |
| | TGDGGFLVQMLSRECHKNREMLSALQTLSRDPDIFPRQTEKTTDKKKTSLY |
| | RKKDGAHPQKKPRTRRAPRKARRSYSSSSSSGSSQSTSRSSSDSESNSDTSSK |
| | KSSKRKRVST (SEQ ID NO: 913) |
| ORF2/3 | MSIWRPPLGNVSYRERNWLQAVETSHSSFCGCGDFILHLTNLAARFALQGPP |
| | PEGGPPRPRPPLLRA |
| | LPAPEVRRETRTENRGASGEPWPGDGGGRDDGAAAGGPADGGDAYDAGD |
| | LDDLFAAVEEEQQCYPESVTKTGRCSPLYKPFQETQISSPDRQRRLPTRRRLR |
| | FTGKKTAHIHRRSPGRGEPPGKRAAPTAAAAAAAALSPPRGAAATRSPTPIH |
| | PPRSPQNASGSPPKPPIIRPATNKVYLFEPSKGLLPIVGKEAWEDEYCTCKYW |
| | DRPPRTNHLDIPTYPWMPTNFKVSFKLGFKP (SEQ ID NO: 914) |
| TAIP | MSPTGREIGFRPSKHPTVLFVAVVILFFILLIWLHALLSRGPRQRVVHLGRGR |
| | RS (SEQ ID NO: 915) |
| ORF1 | MAWRWWWQRRWRRRRWPRRRWRRLRRRRPRRPVRRRRRRTTVRRRRW |
| | RGRRGRRTYTRRAVRRRRRPRKRLVLTQWSPQTVRNCSIRGIVPMVICGHT |
| | KAGRNYAIHSEDFTTQIQPFGGSFSTTTWSLKVLWDEHQKFQNRWSYPNTQ |
| | LDLARYRGVTFWFYRDQKTDYIVQWSRNPPFKLNKYSSAMYHPGMMMQA |
| | KRKLVVPSFQTRPKGKKRYRVTIKPPNMFADKWYTQEDLCPVPLVQIVVSA |
| | ASLLHPFCPPQTNNPCITFQVLKDIYDECIGVNETMKDKYKKLQTTLYTTCT |
| | YYQTTQVLAQLSPAFQPAMKPTTTQSAATATTLGNYVPELKYNNGSFHTGQ |
| | NAVFGMCSYKPTDSIMTKANGWFWQNLMVDNNLHSSYGKATLECMEYHT |
| | GIYSSIFLSPQRSLEFPAAYQDVTYNPNCDRAVGNVVWFQYSTKMDTNFDE |
| | TKCKCVLKNIPLWAAFNGYSDFIMQELSISTEIHNFGIVCFQCPYTFPPCENK |
| | NKPLKGYVFYDTTFGNGKMPDGSGHVPIYWQQRWWIRLAFQVQVMHDFV |
| | LTGPFSYKDDLANTTLTARYKFKFKWGGNIIPEQIIKNPCHREQSLASYPDRQ |
| | RRDLQVVDPSTMGPIYTFHTWDWRRGLFGADAIQRVSQKPGDALRFTNPFK |
| | RPRYLPPTDREDYRQEEDFALQEKRRRTSTEEAQDEESPPESAPLLQQQQQQ |
| | RQLSVHLAEQQRLGVQLRYILQEVLKTQAGLHLNPLLLGPPQTRSISLSPPKA |
| | YSP (SEQ ID NO: 916) |
| ORF1/1 | MAWRWWWQRRWRRRRWPRRRWRRLRRRRPRRPVRRRRRRTTIIKNPCHR |
| | EQSLASYPDRQRRDLQVVDPSTMGPIYTFHTWDWRRGLFGADAIQRVSQKP |
| | GDALRFTNPFKRPRYLPPTDREDYRQEEDFALQEKRRRTSTEEAQDEESPPE |
| | SAPLLQQQQQQRQLSVHLAEQQRLGVQLRYILQEVLKTQAGLHLNPLLLGP |
| | PQTRSISLSPPKAYSP (SEQ ID NO: 917) |
| ORF1/2 | MAWRWWWQRRWRRRRWPRRRWRRLRRRRPRRPVRRRRRRTTMLSRECH |
| | KNREMLSALQTLSRDPDIFPRQTEKTTDKKKTSLYRKKDGAHPQKKPRTRR |
| | APRKARRSYSSSSSSGSSQSTSRSSSDSESNSDTSSKKSSKRKRVST (SEQ ID |
| | NO: 918) |

TABLE N21
Exemplary Anellovirus nucleic acid
sequence (Betatorquevirus)

| | |
|---|---|
| Name | Ring9 |
| Genus/Clade | Betatorquevirus |
| Accession Number | MH649263.1 |

Full Sequence: 2845 bp
1 10 20 30 40 50
| | | | | |
TTATTAATATTCAACAGGAAAACCACCTAATTTAAATTGCCGACCACAAA
CCGTCACTAACTTCCTTATTTAACATTACTTCCCTTTTAACCAATGAATA
TTCATACAACACATCACACTTCCTGGGAGGAGACATAAAACTATATAACT
AACTACACAGACGAATGGCTGAGTTTATGCCGCTAGACGGAGGACGCACA
GCTACTGCTGCGACCTGAACTTGGGCGGGTGCCGAAGGTGAGTGTAACCA
CCGTAGTCAAGGGGCAATTCGGGCTAGTTCAGTCTAGCGGAACGGGCAAG
ATTATTAATACAAACTTATTTTTACAGATGAGCAAACAACTAAAACCAAC
TTTATACAAAGACAAATCATTGGAATTACAATGGCTAAACAACATTTTTA
GCTCTCACGACCTGTGCTGCGGCTGCAACGATCCAGTTTTACATTTACTG
ATTTTAATTAACAAAACCGGAGAAGCACCTAAACCAGAAGAAGACATTAA
AAATATAAAATGCCTCCTTACTGGCGCCAAAAATACTACCGAAGAAGATA
TAGACCTTTCTCCTGGAGAACTAGAAGAATTATTCAAAGAAGAAAAAGAT
GGAGATACCGCAAACCAAGAAAAACATACTGGAGAAGAAAACTGCGGGTA
AGAAAACGTTTTTATAAAAGAAAGTTAAAAAAAATTGTACTTAAACAGTT
TCAACCAAAAATTATTAGAAGATGTACAATATTTGGAACAATCTGCCTAT
TTCAAGGCTCTCCAGAAAGAGCCAACAATAATTATATTCAAACAATCTAC
TCCTACGTACCAGATAAAGAACCAGGAGGAGGGGGATGGACTTTAATAAC
TGAAAGCTTAAGTAGTTTATGGGAAGACTGGGAACATTTAAAAAATGTAT
GGACTCAAAGTAACGCTGGTTTACCACTTGTAAGATACGGGGGAGTAACA
TTATACTTTTATCAATCTGCCTATACTGACTATATTGCTCAAGTTTTCAA
CTGTTATCCTATGACAGACACAAAATACACACATGCAGACTCAGCACCAA
ACAGAATGTTATTAAAAAAACATGTAATAAGAGTACCTAGCAGAGAAACA
CGCAAAAAAAGAAAGCCATACAAAAGAGTTAGAGTAGGACCTCCTTCTCA
AATGCAAAACAAATGGTACTTTCAAAGAGACATATGTGAAATACCATTAA
TAATGATTGCAGCCACAGCCGTTGACTTTAGATATCCCTTTTGTGCAAGC
GACTGTGCTAGTAACAACTTAACTCTAACATGTTTAAACCCACTATTGTT
TCAAAACCAAGACTTTGACCACCCATCCGATACACAAGGCTACTTTCCAA
AACCTGGAGTATATCTATACTCAACACAAAGAAGTAACAAGCCAAGTTCT
TCAGACTGTATATACTTAGGAAACACAAAAGACAATCAAGAAGGTAAATC
TGCAAGTAGTCTAATGACTCTAAAAACACAAAAAATAACAGATTGGGGAA
ATCCATTTTGGCATTATTATATAGACGGTTCTAAAAAAATATTTTCTTAC
TTTAAACCCCCATCACAATTAGACAGCAGCGACTTTGAACACATGACAGA
ATTAGCAGAACCAATGTTTATACAAGTTAGATACAACCCAGAAAGAGACA
CAGGACAAGGAAACTTAATATACGTAACAGAAAACTTTAGAGGACAACAC
TGGGACCCTCCATCTAGTGACAACCTAAAATTAGATGGATTTCCCTTATA
TGACATGTGCTGGGGTTTCATAGACTGGATAGAAAAAGTTCATGAAACAG
AAAACTTACTTACCAACTACTGCTTCTGTATTAGAAGCAGCGCTTTCAAT
GAAAAAAAAACAGTTTTTATACCTGTAGATCATTCATTTTTAACAGGTTT
TAGCCCATATGAAACTCCAGTTAAATCATCAGACCAAGCTCACTGGCACC
CACAAATAAGATTTCAAACAAAATCAATAAATGACATTTGTTTAACAGGC
CCCGGTTGTGCTAGGTCCCCATATGGCAATTACATGCAGGCAAAAATGAG
TTATAAATTTCATGTAAAATGGGGAGGATGTCCAAAAACTTATGAAAAAC
CATATGATCCTTGTTCACAGCCCAATTGGACTATTCCCCATAACCTCAAT
GAAACAATACAAATCCAGAATCCAAACACATGCCCACAAACAGAACTCCA
AGAATGGGACTGGCGACGTGATATTGTTACAAAAAAAGCTATCGAAAGAA
TTAGACAACACACGGAACCTCATGAAACTTTGCAAATCTCTACAGGTTCC
AAACACAACCCACCAGTACACAGACAAACATCACCGTGGACGGACTCAGA
AACGGACTCGGAAGAGGAAAAAGACCAAACACAAGAGATCCAGATCCAGC
TCAACAAGCTCAGAAAGCATCAACAGCATCTCAAGCAGCAGCTCAAGCAG
TACCTGAAACCCCAAAATATAGAATAGTTGCAAGCAACATAAAAGTTGAA
CTTTTTCCTACTAAAAAACCTTTTAAAAACAGACGCTTTACTCCTTCTGA
AAGAGAAACAGAAAGACAATGTGCTAAAGCTTTTTGTAGACCAGAAAGAC
ATTTCTTTTATGATCCTCCTTTTTACCCTTACTGTGTACCTGAACCTATT
GTAAACTTTGCTTTGGGATATAAAATTTAAGGCCAACAAATTTCACTTAG
TGGTGTCTGTTTATTAAAGTTTAACCTTAATAAGCATACTCCGCCTCCCT
ACATTAAGGCGCCAAAAGGGGGCTCCGCCCCCTTAAACCCCAAGGGGGCT
CCGCCCCCTTAAACCCCCAAGGGGGCTCCGCCCCCTTACACCCCC (SEQ
ID NO: 1001)
Annotations:

| Putative Domain | Base range |
|---|---|
| TATA Box | 142-148 |
| Initiation Element | 162-177 |
| Transcriptional Start Site | 172 |
| 5′ UTR Conserved Domain | 226-296 |
| ORF2 | 328-651 |
| ORF2/2 | 328-647; 2121-2457 |
| ORF2/3 | 328-647; 2296-2680 |
| ORF1 | 510-2477 |
| ORF1/1 | 510-647; 2121-2477 |
| ORF1/2 | 510-647; 2296-2457 |
| Three open-reading frame region | 2296-2454 |
| GC-rich region | 2734-2845 |

TABLE A21
Exemplary Anellovirus amino acid sequences
(Betatorquevirus)
Ring9 (Betatorquevirus)

| | |
|---|---|
| ORF2 | MSKQLKPTLYKDKSLELQWLNNIFSSHDLCCGCNDPVLHLLILINKTGEAPK |
| | PEEDIKNIKCLLTGAKNTTEEDIDLSPGELEELFKEEKDGDTANQEKHTGEE |
| | NCG (SEQ ID NO: 1002) |
| ORF2/2 | MSKQLKPTLYKDKSLELQWLNNIFSSHDLCCGCNDPVLHLLILINKTGEAPK |
| | PEEDIKNIKCLLTGAKNTTEEDIDLSPGELEELFKEEKDGDTANQEKHTGEE |
| | NCGPIGLFPITSMKQYKSRIQTHAHKQNSKNGTGDVILLQKKLSKELDNTRN |
| | LMKLCKSLQVPNTTHQYTDKHHRGRTQKRTRKRKKTKHKRSRSSSTSSESIN |
| | SISSSSSSST (SEQ ID NO: 1003) |
| ORF2/3 | MSKQLKPTLYKDKSLELQWLNNIFSSHDLCCGCNDPVLHLLILINKTGEAPK |
| | PEEDIKNIKCLLTGAKNTTEEDIDLSPGELEELFKEEKDGDTANQEKHTGEE |
| | NCGFQTQPTSTQTNITVDGLRNGLGRGKRPNTRDPDPAQQAQKASTASQAAA |
| | QAVPETPKYRIVASNIKVELFPTKKPFKNRRFTPSERETERQCAKAFCRPER |
| | HFFYDPPFYPYCVPEPIVNFALGYKI (SEQ ID NO: 1004) |
| ORF1 | MPPYWRQKYYRRRYRPFSWRTRRIIQRRKRWRYRKPRKTYWRRKLRVRKRFY |
| | KRKLKKIVLKQFQPKIIRRCTIFGTICLFQGSPERANNNYIQTIYSYVPDKE |
| | PGGGGWTLITESLSSLWEDWEHLKNVWTQSNAGLPLVRYGGVTLYFYQSAYT |
| | DYIAQVENCYPMTDTKYTHADSAPNRMLLKKHVIRVPSRETRKKRKPYKRVR |
| | VGPPSQMQNKWYFORDICEIPLIMIAATAVDFRYPFCASDCASNNLTLTCLN |
| | PLLFQNQDFDHPSDTQGYFPKPGVYLYSTQRSNKPSSSDCIYLGNTKDNQEG |
| | KSASSLMTLKTQKITDWGNPFWHYYIDGSKKIFSYFKPPSQLDSSDFEHMTE |
| | LAEPMFIQVRYNPERDTGQGNLIYVTENFRGQHWDPPSSDNLKLDGFPLYDM |
| | CWGFIDWIEKVHETENLLTNYCFCIRSSAFNEKKTVFIPVDHSFLTGFSPYE |
| | TPVKSSDQAHWHPQIRFQTKSINDICLTGPGCARSPYGNYMQAKMSYKFHVK |
| | WGGCPKTYEKPYDPCSQPNWTIPHNLNETIQIQNPNTCPQTELQEWDWRRDI |
| | VTKKAIERIRQHTEPHETLQISTGSKHNPPVHRQTSPWTDSETDSEEEKDQT |
| | QEIQIQLNKLRKHQQHLKQQLKQYLKPQNIE (SEQ ID NO: 1005) |
| ORF1/1 | MPPYWRQKYYRRRYRPFSWRTRRIIQRRKRWRYRKPRKTYWRRKLRPNWTIP |
| | HNLNETIQIQNPNTCPQTELQEWDWRRDIVTKKAIERIRQHTEPHETLQIST |
| | GSKHNPPVHRQTSPWTDSETDSEEEKDQTQEIQIQLNKLRKHQQHLKQQLKQ |
| | YLKPQNIE (SEQ ID NO: 1006) |
| ORF1/2 | MPPYWRQKYYRRRYRPFSWRTRRIIQRRKRWRYRKPRKTYWRRKLRVPNTTH |
| | QYTDKHHRGRTQKRTRKRKKTKHKRSRSSSTSSESINSISSSSSSST (SEQ |
| | ID NO: 1007) |

TABLE N22
Exemplary Anellovirus nucleic acid
sequence (Betatorquevirus)

| | |
|---|---|
| Name | Ring10 |
| Genus/Clade | Betatorquevirus |
| Accession Number | JX134044.1 |

Full Sequence: 2912 bp
1 10 20 30 40 50
| | | | | |
TAATAAATATTCAACAGGAAAACCACCTAATTTAAATTGCCGACCACAAA
CCGTCACTTAGTTCCTCTTTTTCCACAACTTCCTCTTTTACTAATGAATA
TTCATGTAATTAATTAATAATCACCGTAATTCCGGGGAGGAGCCTTTAAA
CTATAAAACTAACTACACATTCGAATGGCTGAGTTTATGCCGCCAGACGG
AGACGGGATCACTTCAGTGACTCCAGGCTGATCAAGGGCGGGTGCCGAAG
GTGAGTGAAACCACCGTAGTCAAGGGGCAATTCGGGCTAGATCAGTCTGG
CGGAACGGGCAAGAAACTTAAAATGTACTTTATTTTACAGAAATGTTCAA
ATCTCCAACATACTTAACAACTAAAGGCAAAAACAATGCCTTAATCAACT
GCTTCGTTGGAGACCACGATCTTCTGTGCAGCTGTAACAATCCTGCCTAC
CATTGCCTCCAAATACTTGCAACTACCTTAGCACCTCAACTAAAACAAGA
AGAAAAACAACAAATAATACAATGCCTTGGTGGTACAGACGCCGTAGCTA
CAACCCGTGGAGACGAAGAAATTGGTTTAGAAGACCTAGAAAAACTATTT
ACAGAAGATACAGAAGAAGACGCCGCTGGGTAAGAAGAAAACCTTTTTAC
AAACGTAAAATTAAGAGACTAAATATAGTAGAATGGCAACCTAAATCAAT
TAGAAAATGTAGAATAAAAGGAATGCTATGCTTGTTTCAAACGACAGAAG
ACAGACTGTCATATAACTTTGATATGTATGAAGAGTCTATTATACCAGAA
AAACTGCCGGGAGGGGGGGGATTTAGCATTAAGAATATAAGCTTATATGC
CTTATACCAAGAACACATACATGCACACAACATATTTACACACACAAACA
CAGACAGACCACTAGCAAGATACACAGGCTGTTCTTTAAAATTCTACCAA
AGCAAAGACATAGACTACGTAGTAACATATTCTACATCACTCCCACTAAG
AAGCTCAATGGGAATGTACAACTCCATGCAACCATCCATACATCTAATGC
AACAAAACAAACTAATTGTACCAAGCAAACAAACACAAAAAAGAAGAAAA
CCATATATTAAAAAACATATATCACCACCAACACAAATGAAATCTCAATG
GTACTTTCAACATAACATTGCAAACATACCGCTACTAATGATAAGAACCA
CAGCATTAACATTAGATAATTACTATATAGGAAGCAGACAATTAAGTACA
AATGTCACTATACATACACTTAACACAACATACATCCAAAACAGAGACTG
GGGAGACAGAAATAAAACTTACTACTGCCAAACATTAGGAACACAAAGAT
ACTTCCTATATGGAACACATTCAACTGCACAAAATATTAATGACATAAAG
CTACAAGAACTAATACCTTTAACAAACACACAAGACTATGTACAAGGCTT
TGATTGGACAGAAAAAGACAAACATAACATAACAACCTACAAAGAATTCT
TAACTAAAGGAGCAGGAAATCCATTTCACGCAGAATGGATAACAGCACAA
AACCCAGTAATACACACAGCAAACAGTCCTACACAAATAGAACAAATATA
CACCGCTTCAACAACAACATTCCAAAACAAAAAACTAACAGACCTACCAA
CGCCAGGATATATATTTATAACTCCAACAGTAAGCTTAAGATACAACCCA
TACAAAGACCTAGCAGAAAGAAACAAATGCTACTTTGTAAGAAGCAAAAT
AAATGCACACGGGTGGGACCCAGAACAACACCAAGAATTAATAAACAGTG
ACCTACCACAATGGTTACTATTATTTGGCTACCCAGACTACATAAAAAGA
ACACAAAACTTTGCATTAGTAGACACAAATTACATACTAGTAGACCACTG
CCCATACACAAATCCAGAAAAAACACCATTTATACCTTTAAGCACATCAT
TTATAGAAGGTAGAAGCCCATACAGTCCTTCAGACACACATGAACCAGAT
GAAGAAGACCAAAACAGGTGGTACCCATGCTACCAATATCAACAAGAATC
AATAAATTCAATATGTCTTAGCGGTCCAGGCACACCAAAAATACCAAAAG
GAATAACAGCAGAAGCAAAAGTAAAATATTCCTTTAATTTTAAGTGGGGT
GGTGACCTACCACCAATGTCTACAATTACAAACCCGACAGACCAGCCAAC
ATATGTTGTTCCCAATAACTTCAATGAAACAACTTCGTTACAGAATCCAA
CCACCAGACCAGAGCACTTCTTGTACTCCTTTGACGAAAGGAGGGGACAA
CTTACAGAAAAAGCTACAAAACGCTTGCTTAAAGACTGGGAAACTAAAGA
AACTTCTTTATTGTCTACAGAATACAGATTCGCGGAGCCAACACAAACAC
AAGCCCCACAAGAGGACCCGTCCTCGGAAGAAGAAGAAGAGAGCAACCTC
TTCGAGCGACTCCTCCGACAGCGAACCAAGCAGCTCCAGCTCAAGCGCAG
AATAATACAAACATTGAAAGACCTACAAAAATTAGAATAACTAACAGCAA
AAACACCGTTTACCTATTTCCACCTGAACAAAAGAACAGAAGACTAACAC
CATGGGAAATACAAGAAGACAAAGAAATAGCCAATTTATTTGGCAGACCA
CATAGATACTTTTTAAAAGACATTCCTTTCTATTGGGATATACCCCCAGA
GCCTAAAGTAAACTTTGATTTAAATTTTCAATAAAGAAATAAAGGGCAAG
GCCCCATTAACTCAAAGTCGGTGTCTACCTCTTTAAGTTTAACTTTACTA
AACGGACTCCGCCTCCCTAAATTTGGGCGCCAAAAGGGGGCTCCGCCCCC
TTAAACCCCAGGGGGCTCCGCCCCCTAAAACCCCCAAGGGGGCTACGCCC
CCTTACACCCCC (SEQ ID NO: 1008)
Annotations:

| Putative Domain | Base range |
|---|---|
| TATA Box | 152-158 |
| Initiation Element | 172-187 |
| Transcriptional Start Site | 182 |
| 5′ UTR Conserved Domain | 239-309 |
| ORF2 | 343-633 |
| ORF2/2 | 343-629; 2196-2505 |
| ORF2/3 | 343-629; 2371-2734 |
| ORF1 | 522-2540 |
| ORF1/1 | 522-629; 2196-2540 |
| ORF1/2 | 522-629; 2371-2505 |
| Three open-reading frame | 2276-2502 |
| region | |
| GC-rich region | 2803-2912 |

TABLE A22
Exemplary Anellovirus amino acid
sequences (Betatorquevirus)
Ring10 (Betatorquevirus)

| | |
|---|---|
| ORF2 | MFKSPTYLTTKGKNNALINCFVGDHDLLCSCNNPAYHCLQILATTLAPQLKQ |
| | EEKQQIIQCLGGTDAVATTRGDEEIGLEDLEKLFTEDTEEDAAG (SEQ ID |
| | NO: 1009) |
| ORF2/2 | MFKSPTYLTTKGKNNALINCFVGDHDLLCSCNNPAYHCLQILATTLAPQLKQ |
| | EEKQQIIQCLGGTDAVATTRGDEEIGLEDLEKLFTEDTEEDAAGQHMLFPIT |
| | SMKQLRYRIQPPDQSTSCTPLTKGGDNLQKKLQNACLKTGKLKKLLYCLQNT |
| | DSRSQHKHKPHKRTRPRKKKKRATSSSDSSDSEPSSSSSSAE (SEQ ID |
| | NO: 1010) |
| ORF2/3 | MFKSPTYLTTKGKNNALINCFVGDHDLLCSCNNPAYHCLQILATTLAPQLKQ |
| | EEKQQIIQCLGGTDAVATTRGDEEIGLEDLEKLFTEDTEEDAAGIQIRGANT |
| | NTSPTRGPVLGRRRREQPLRATPPTANQAAPAQAQNNTNIERPTKIRITNSK |
| | NTVYLFPPEQKNRRLTPWEIQEDKEIANLFGRPHRYFLKDIPFYWDIPPEPK |
| | VNFDLNFQ (SEQ ID NO: 1011) |
| ORF1 | MPWWYRRRSYNPWRRRNWFRRPRKTIYRRYRRRRRWVRRKPFYKRKIKRLNI |
| | VEWQPKSIRKCRIKGMLCLFQTTEDRLSYNFDMYEESIIPEKLPGGGGFSIK |
| | NISLYALYQEHIHAHNIFTHTNTDRPLARYTGCSLKFYQSKDIDYVVTYSTS |
| | LPLRSSMGMYNSMQPSIHLMQQNKLIVPSKQTQKRRKPYIKKHISPPTQMKS |
| | QWYFQHNIANIPLLMIRTTALTLDNYYIGSRQLSTNVTIHTLNTTYIQNRDW |
| | GDRNKTYYCQTLGTQRYFLYGTHSTAQNINDIKLQELIPLTNTQDYVQGFDW |
| | TEKDKHNITTYKEFLTKGAGNPFHAEWITAQNPVIHTANSPTQIEQIYTAST |
| | TTFQNKKLTDLPTPGYIFITPTVSLRYNPYKDLAERNKCYFVRSKINAHGWD |
| | PEQHQELINSDLPQWLLLFGYPDYIKRTQNFALVDTNYILVDHCPYTNPEKT |
| | PFIPLSTSFIEGRSPYSPSDTHEPDEEDQNRWYPCYQYQQESINSICLSGPG |
| | TPKIPKGITAEAKVKYSFNFKWGGDLPPMSTITNPTDQPTYVVPNNFNETTS |
| | LQNPTTRPEHFLYSFDERRGQLTEKATKRLLKDWETKETSLLSTEYRFAEPT |
| | QTQAPQEDPSSEEEEESNLFERLLRQRTKQLQLKRRIIQTLKDLQKLE |
| | (SEQ ID NO: 1012) |
| ORF1/1 | MPWWYRRRSYNPWRRRNWFRRPRKTIYRRYRRRRRWPTYVVPNNFNETTSLQ |
| | NPTTRPEHFLYSFDERRGQLTEKATKRLLKDWETKETSLLSTEYRFAEPTQT |
| | QAPQEDPSSEEEEESNLFERLLRQRTKQLQLKRRIIQTLKDLQKLE |
| ORF1/2 | MPWWYRRRSYNPWRRRNWFRRPRKTIYRRYRRRRRWNTDSRSQHKHKPHKRT |
| | RPRKKKKRATSSSDSSDSEPSSSSSSAE (SEQ ID NO: 1013) |

TABLE N23
Exemplary Anellovirus nucleic acid
sequence (Alphatorquevirus, Clade 4)

| | |
|---|---|
| Name | Ring20 |
| Genus/Clade | Alphatorquevirus Clade 4 |
| Accession Number | AF122914.3 |

Full Sequence: 3853 bp
1 10 20 30 40 50
| | | | | |
GGCTTAGTGCGTCACCACCCACGTGACCCGCCTCCGCCAATTAACAGGTA
CTTCGTACACTTCCTGGGCGGGCTTATAAGACTAATATAAGTAGCTGCAC
TTCCGAATGGCTGAGTTTTCCACGCCCGTCCGCAGCGGTGAAGCCACGGA
GGGAGCTCAGCGCGTCCCGAGGGCGGGTGCCGGAGGTGAGTTTACACACC
GCAGTCAAGGGGCAATTCGGGCTCGGGACTGGCCGGGCTTTGGGCAAGGC
TCTTAAAAAAGCTATGTTTATTGGCAGGCACTACCGAAAGAAAAGGGCGC
TGCTACTGCTATCTGTGCATTCTACAAAGACAAAAGGGAAACTTCTAATA
GCTATGTGGACTCCCCCACGCAATGATCAACAATACCTTAACTGGCAATG
GTACACTTCTGTACTTAGCTCCCACTCTGCTATGTGCGGGTGTTCCGACG
CTATCGCTCATCTTAATCATCTTGCTAATCTGCTTCGTGCCCCGCAAAAT
CCGCCCCCGCCTGATAATCCAAGACCCCTACCCGTGCGAGCACTGCCTGC
TCCCCCGGCTGCCCACGAGGCAGCCGGTGATCGAGCACCATGGCCTATGG
GTGGTGGAGGAGACGCCGGAGGCGCTGGCGCAGGTGGAGACGCCGACCAT
GGAGGCGCCGCTGGAGGACCCGCAGACGCAGACCTGCTAGACGCCGTGGC
CGCCGCAGAAACGTAAGGAGACGGCGCAGAGGGAGGTGGAGAAGGAGGTA
CAGGAGGTGGAAAAGAAAGGGCAGACGTAGAAGAAAAGCAAAAATAATAA
TAAGACAGTGGCAGCCAAACTACAGAAGAAGATGTAATATAGTGGGCTAC
CTCCCTATACTTATCTGTGGTGGAAATACTGTTTCTAGAAACTATGCCAC
ACACTCAGACGATACTAACTATCCAGGACCCTTTGGGGGAGGCATGACCA
CAGACAAATTCAGCCTTAGAATACTATATGATGAATACAAAAGATTTATG
AACTACTGGACAGCCTCAAATGAGGACCTAGATCTCTGTAGATATCTAGG
ATGCACTTTTTACTTCTTTAGACACCCTGAAGTAGACTTTATTATAAAAA
TAAACACCATGCCCCCATTCTTAGATACAACCATAACAGCACCTAGCATA
CACCCAGGCCTCATGGCCCTAGACAAAAGAGCCAGATGGATTCCTTCTCT
TAAAAATAGACCAGGTAAAAAACACTATATAAAAATTAGAGTAGGGGCTC
CTAAAATGTTCACAGATAAATGGTACCCTCAAACAGACCTCTGTGACATG
ACACTGCTAACTATCTATGCAACCGCAGCGGATATGCAATATCCGTTCGG
CTCACCACTAACTGACACTGTGGTTGTTAACTCCCAAGTTCTGCAATCCA
TGTATGATGAAACAATTAGCATATTACCTGATGAAAAAACTAAAAGAAAT
AGCCTTCTTACTTCTATAAGAAGCTACATACCTTTTTATAATACTACACA
AACAATAGCTCAATTAAAACCATTTGTAGATGCAGGAGGACACACAACAG
GCTCAACAACAACTACATGGGGACAACTATTAAACACAACTAAATTTACC
ACTACCACAACAACCACATACACATACCCTGGCACCACAAATACAGCAGT
AACATTTATAACAGCCAATGATACCTGGTACAGGGGAACAGCATATAAAG
ATAACATTAAAGATGTACCACAAAAAGCAGCACAATTATACTTTCAAACA
ACACAAAAACTACTAGGAAACACATTCCATGGCTCAGATGAAACACTTGA
ATACCATGCAGGCCTATACAGCTCTATCTGGCTATCACCAGGTAGATCCT
ACTTTGAAACACCAGGTGCATACACAGACATTAAATATAACCCTTTTACA
GACAGAGGAGAAGGCAACATGCTGTGGATAGACTGGCTAAGTAAAAAAAA
CATGAAATATGACAAAGTGCAAAGTAAGTGCCTAGTAGCAGACCTACCAC
TGTGGGCAGCAGCATATGGTTATGTAGAATTCTGCTCTAAAAGCACAGGA
GACACAAACATACACATGAATGCCAGACTACTAATAAGAAGTCCTTTTAC
AGACCCCCAGCTAATAGTACACACAGACCCCACTAAAGGCTTTGTACCCT
ATTCTTTAAACTTTGGAAATGGTAAAATGCCAGGAGGTAGCAGCAATGTT
CCCATAAGAATGAGAGCTAAGTGGTACCCCACTTTATCCCACCAACAAGA
AGTTCTAGAGGCCTTAGCACAGTCAGGACCCTTTGCTTATCACTCAGACA
TTAAAAAAGTATCTCTAGGCATAAAATACCGTTTTAAGTGGATCTGGGGT
GGAAACCCCGTTCGCCAACAGGTTGTTAGAAATCCCTGCAAGGAACCCCA
CTCCTCGGGCAATAGAGTCCCTAGAAGCATACAAATCGTTGACCCGAGAT
ACAACTCACCGGAACTTACCATCCATGCCTGGGACTTCAGACGTGGCTTC
TTTGGCCCGAAAGCTATTCAAAGAATGCAACAACAACCAACTGCTACTGA
ATTTTTTTCAGCAGGCCGCAAGAGACCCAGAAGGGACACAGAAGTGTATC
AGTCCGACCAAGAAAAGGAGCAAAAAGAAAGCTCGCTTTTCCCCCCAGTC
AAGCTCCTCCGAAGAGTCCCCCCGTGGGAGGACTCGGAACAGGAGCAAAG
CGGGTCGCAAAGCTCAGAGGAAGAGACGGCGACCCTCTCCCAGCAGCTCA
AACAGCAGCTGCAGCAGCAGCGAGTCTTGGGAGTCAAACTCAGACTCCTG
TTCAACCAAGTCCAAAAAATCCAACAAAATCAAGATATCAACCCTACCTT
GTTACCAAGGGGGGGGGATCTAGTATCCTTCTTTCAGGCTGTACCATAAA
TATGTTTCCAGACCCTAAACCTTACTGCCCCTCCAGCAATGACTGGAAAG
AAGAGTATGAGGCCTGTAAATATTGGGATAGACCTCCCAGACACAACCTT
AGAGACCCCCCCTTTTACCCCTGGGCCCCTAAAAACAATCCTTGCAATGT
AAGCTTTAAACTTGGCTTCAAATAAACTAGGCCGTGGGAGTTTCACTTGT
CGGTGTCTACCTCTATAAGTCACTAAGCACTCCGAGCGCAGCGAGGAGTG
CGACCCTTCCCCCTGGTGCAACGCCCTCGGCGGCCGCGCGCTACGCCTTC
GGCTGCGCGCGGCACCTCGGACCCCCGCTCGTGCTGACACGCTTGCGCGT
GTCAGACCACTTCGGGCTCGCGGGGGTCGGGAAATTTGCTAAACAGACTC
CGAGTTGCCATTGGACACTGTAGCTATGAATCAGTAACGAAAGTGAGTGG
GGCCAGACTTCGCCATAAGGCCTTTATCTTCTTGCCATTTGTCAGTATTG
GGGGTCGCCATAAACTTTGGGCTCCATTTTAGGCCTTCCGGACTACAAAA
ATCGCCATATTTGTGACGTCAGAGCCGCCATTTTAAGTCAGCTCTGGGGA
GGCGTGACTTCCAGTTCAAAGGTCATCCTCACCATAACTGGCACAAAATG
GCCGCCAACTTCTTCCGGGTCAAAGGTCACTGCTACGTCATAGGTGACGT
GGGGGGGGACCTACTTAAACACGGAAGTAGGCCCCGACACGTCACTGTCA
CGTGACAGTACGTCACAGCCGCCATTTTGTTTTACAAAATAGCCGACTTC
CTTCCTCTTTTTTAAAAAAAGGCGCCAAAAAACCGTCGGCGGGGGGGCCG
CGCGCTGCGCGCGCGGCCCCCGGGGGAGGCACAGCCTCCCCCCCCCGCGC
GCATGCGCGCGGGTCCCCCCCCCTCCGGGGGGCTCCGCCCCCCGGCCCCC
CCC (SEQ ID NO: 1014)
Annotations:

| Putative Domain | Base range |
|---|---|
| TATA Box | 86-90 |
| Initiation Element | 104-119 |
| Transcriptional Start | 114 |
| Site | |
| 5′ UTR Conserved Domain | 174-244 |
| ORF2 | 354-716 |
| ORF2/2 | 354-712; 2372-2873 |
| ORF2/3 | 354-712; 2565-3075 |
| ORF2t/3 | 354-400; 2565-3075 |
| TAIP | 373-690 |
| ORF1 | 590-2899 |
| ORF1/1 | 590-712; 2372-2899 |
| ORF1/2 | 590-712; 2565-2873 |
| Three open-reading | 2551-2870 |
| frame region | |
| Poly(A)-Signal | 3071-3076 |
| GC-rich region | 3733-3853 |

TABLE A23
Exemplary Anellovirus amino acid
sequences (Alphatorquevirus)
Ring20 (Alphatorquevirus Clade 4)

| ORF2 | MWTPPRNDQQYLNWQWYTSVLSSHSAMCGCSDAIAHLNHLANLLRAPQNPPP |
|---|---|
| | PDNPRPLPVRALPAPPAAHEAAGDRAPWPMGGGGDAGGAGAGGDADHGGAAG |
| | GPADADLLDAVAAAET (SEQ ID NO: 1015) |
| ORF2/2 | MWTPPRNDQQYLNWQWYTSVLSSHSAMCGCSDAIAHLNHLANLLRAPQNPPP |
| | PDNPRPLPVRALPAPPAAHEAAGDRAPWPMGGGGDAGGAGAGGDADHGGAAG |
| | GPADADLLDAVAAAETLLEIPARNPTPRAIESLEAYKSLTRDTTHRNLPSMP |
| | GTSDVASLARKLFKECNNNQLLLNFFQQAARDPEGTQKCISPTKKRSKKKAR |
| | FSPQSSSSEESPRGRTRNRSKAGRKAQRKRRRPSPSSSNSSCSSSESWESNS |
| | DSCSTKSKKSNKIKISTLPCYQGGGI (SEQ ID NO: 1016) |
| ORF2/3 | MWTPPRNDQQYLNWQWYTSVLSSHSAMCGCSDAIAHLNHLANLLRAPQNPPP |
| | PDNPRPLPVRALPAPPAAHEAAGDRAPWPMGGGGDAGGAGAGGDADHGGAAG |
| | GPADADLLDAVAAAETPQETQKGHRSVSVRPRKGAKRKLAFPPSQAPPKSPP |
| | VGGLGTGAKRVAKLRGRDGDPLPAAQTAAAAAASLGSQTQTPVQPSPKNPTK |
| | SRYQPYLVTKGGGSSILLSGCTINMFPDPKPYCPSSNDWKEEYEACKYWDRP |
| | PRHNLRDPPFYPWAPKNNPCNVSFKLGFK (SEQ ID NO: 1017) |
| ORF2t/3 | MWTPPRNDQQYLNWQWPQETQKGHRSVSVRPRKGAKRKLAFPPSQAPPKSPP |
| | VGGLGTGAKRVAKLRGRDGDPLPAAQTAAAAAASLGSQTQTPVQPSPKNPTK |
| | SRYQPYLVTKGGGSSILLSGCTINMFPDPKPYCPSSNDWKEEYEACKYWDRP |
| | PRHNLRDPPFYPWAPKNNPCNVSFKLGFK (SEQ ID NO: 1018) |
| TAIP | MINNTLTGNGTLLYLAPTLLCAGVPTLSLILIILLICFVPRKIRPRLIIQDP |
| | YPCEHCLLPRLPTRQPVIEHHGLWVVEETPEALAQVETPTMEAPLEDPQTQT |
| | C (SEQ ID NO: 1019) |
| ORF1 | MAYGWWRRRRRRWRRWRRRPWRRRWRTRRRRPARRRGRRRNVRRRRRGRWRR |
| | RYRRWKRKGRRRRKAKIIIRQWQPNYRRRCNIVGYLPILICGGNTVSRNYAT |
| | HSDDTNYPGPFGGGMTTDKFSLRILYDEYKRFMNYWTASNEDLDLCRYLGCT |
| | FYFFRHPEVDFIIKINTMPPFLDTTITAPSIHPGLMALDKRARWIPSLKNRP |
| | GKKHYIKIRVGAPKMFTDKWYPQTDLCDMTLLTIYATAADMQYPFGSPLTDT |
| | VVVNSQVLQSMYDETISILPDEKTKRNSLLTSIRSYIPFYNTTQTIAQLKPF |
| | VDAGGHTTGSTTTTWGQLLNTTKFTTTTTTTYTYPGTTNTAVTFITANDTWY |
| | RGTAYKDNIKDVPQKAAQLYFQTTQKLLGNTFHGSDETLEYHAGLYSSIWLS |
| | PGRSYFETPGAYTDIKYNPFTDRGEGNMLWIDWLSKKNMKYDKVQSKCLVAD |
| | LPLWAAAYGYVEFCSKSTGDTNIHMNARLLIRSPFTDPQLIVHTDPTKGFVP |
| | YSLNFGNGKMPGGSSNVPIRMRAKWYPTLSHQQEVLEALAQSGPFAYHSDIK |
| | KVSLGIKYRFKWIWGGNPVRQQVVRNPCKEPHSSGNRVPRSIQIVDPRYNSP |
| | ELTIHAWDFRRGFFGPKAIQRMQQQPTATEFFSAGRKRPRRDTEVYQSDQEK |
| | EQKESSLFPPVKLLRRVPPWEDSEQEQSGSQSSEEETATLSQQLKQQLQQQR |
| | VLGVKLRLLFNQVQKIQQNQDINPTLLPRGGDLVSFFQAVP (SEQ ID |
| | NO: 1020) |
| ORF1/1 | MAYGWWRRRRRRWRRWRRRPWRRRWRTRRRRPARRRGRRRNVVRNPCKEPHS |
| | SGNRVPRSIQIVDPRYNSPELTIHAWDFRRGFFGPKAIQRMQQQPTATEFFS |
| | AGRKRPRRDTEVYQSDQEKEQKESSLFPPVKLLRRVPPWEDSEQEQSGSQSS |
| | EEETATLSQQLKQQLQQQRVLGVKLRLLFNQVQKIQQNQDINPTLLPRGGDL |
| | VSFFQAVP (SEQ ID NO: 1021) |
| ORF1/2 | MAYGWWRRRRRRWRRWRRRPWRRRWRTRRRRPARRRGRRRNAARDPEGTQKC |
| | ISPTKKRSKKKARFSPQSSSSEESPRGRTRNRSKAGRKAQRKRRRPSPSSSN |
| | SSCSSSESWESNSDSCSTKSKKSNKIKISTLPCYQGGGI (SEQ ID NO: |
| | 1022) |

TABLE N24
Novel Anellovirus nucleic acid
sequence (Betatorquevirus)

| | |
|---|---|
| Name | RING 19 |
| Genus/Clade | Betatorquevirus |
| Accession | N/A |

Full Sequence: 2876 bp
1 10 20 30 40 50
| | | | | |
CGGGAGCCGAAGGTGAGTGCAACCACCGTAGTCTAGGGGCAATTCGGGCT
AGTTCAGTATGGCGGAACGGGCAAGAAACTTAAATATTATTATTTTACAG
ATGCAAATACAACCACCTATTAGAACCTTCAAACAAACAATTTCAGATTG
GAAAAACTTAATTGTCCACGTTCACGACAACATTTGCAACTGCAATAAAC
CATTAGAACACACTATTGATACCTGTATCACCAATCCAGATGAATTAAGA
TTAAACAAATCTACTAAACAACAACTACAAAAATGCCTTGGTACCCCAGA
AGAAGATACCCAAGAAGACGTTATCGATGGCTTCGCAGATGGAGAGCTAG
ACGCCCTTTTCGCCCAAGATACAGAAGAAGATACTGGGTAAGAAACTATT
CTCGAAAGAGAAAACTATTTAAAATAACAACCAAAGAATGGCAACCAAAA
GTTATAAGAAAGACTCATGTAAAGGGCACCTATCCTTTGTTTCTTTGTAC
AAAGCACAGAATTAACAATAATATGATACAATATTTAGACTCTATAGCTC
CAGAACACTATTACGGAGGAGGAGGATTTTCAATAATGCAATTTTCCTTA
CAAGCCTTATATGAAGAATTTATAAAAGCAAAAAACTGGTGGACTAATAC
AAACTGCTTTTTACCACTTGTAAGATATATGGGTTGCTCATTCAAATTTT
ATAAAACTGAATTTTATGATTATATTGTACTAATTGAAAGATGTTATCCA
CTTGCTTGTACTGATGAAATGTACTTATCTACTCAACCTAGTATTATGAT
GCTTACAAGAAAATGTATTTTTGTACCATGCAAACAAAACAGCAAAGGTA
AAAAACCTTACAAAAAAGTTAGAGTAAGACCACCTTCACAAATGACTACA
GGATGGCATTTCTCACAAGACTTAGCAAACATGCCACTTGTAGTACTAAA
AACTTCAGTATGCAGCTTTGACAGATATTACACAGACAGTACAGCTAAAT
CAACCACAATAGGCTTTAAAACACTTAACACACAAACATTTAGATATCAT
GACTGGCAGGAACCACCTACAACAGGATACAAACCACAAAACCTACTATG
GTTTTATGGAGCAGAAAACGGATCACCAGTAGACCCCAACAACACAATAG
TATCAAACCTAATATACTTAGGAGGCACAGGACCTTATGAAAAAGGCACA
CCAATAAAAACAAACATAAGCAATTACTTTTCAGAGCCTAAACTGTGGGG
AAATATATTTCACGATGATTATACATCAGGAACATCACCCGTGTTTGTTA
CAAACAAATCACCATCAGAAATTAAAACCGCATGGAACACTATAAAAGAC
TTAACTGTTAAAGCTAGCGGTGTATTTACATTAAGAACAATTCCACTATG
GCTACCTTGCAGATACAACCCATTTGCAGACAAAGCAACCAACAACAAAA
TATGGCTAGTTTCTATACATTCAGACCACACAGAATGGAAACCAATAGAC
AATCCATTACTACAACGAACAGACCTTCCTTTATGGTTACTTGTATGGGG
TTGGCAAGATTGGCAGAAAAAAAACCAACAAACTTCACAACCTGATATTA
ATTATTTAACAGTAATATCTTCACCATATATATCATGCTACCCAAAATTA
GATTACTATGTGTTACTAGATGAAGGATTTTGGGAGGGTCACTCAACATA
CATAGAGTCAATTACAGACTCAGACAAAAAACACTGGTACCCTAAAAATA
GATTTCAAATAGAAACACTTAATCTAATAGCTAACACAGGTCCAGGAACT
GTAAAACTAAGAGAAAACCAAGCAGCAGAAGGTCACATGGTATATCGCTT
TAATTTTAAGCTTGGAGGATGTCCCGCACCGATGGAAAAAATATGTGACC
CTAGCAAACAATCCAAATATCCTATTCCCAATAACCAGCAACAAACAACT
TCGTTGCAGAGTCCAGAAAACCCAATTCAAACCTATCTCTACGACTTCGA
CGAAAGGAGGGGCCTACTTACAGAAAGAGCTACAAAAAGAATCAAACAAG
ATCACACATCTGAAAAAACTGTTTTGCCATTTACAGGAGCAGCAACAGAC
CTCCCCATACTCCAAACAACATCACAGGAGGAAAGCTCCTCGGAAGAAGA
AGAAGAGCAACAAGCGGAGAAGAAACTACTCCAGCTCCGAAGAAAGCAGC
ACCGACTCCGGGAGCGAATCCTCCAGCTATTAGACATACAAAATACATAA
TAAAACAAAGTACTGTAAAAATTGATATGTTTGGAGATACTCATGTACCT
AACCGTAGAATGACCCCAGAAGAATTTGAACAAGAACTAATTGTCGCTGG
TGTTTTTCGCAGACCTCCTTGTTACTATATAAAAGATAGACCTACTTATC
CTTATGTACCAAAACCTACTGATGAAAAATGTATGGTAAACTTTGACTTA
AACTTTCCTTAATAAACTACGCCTGCAAACTTTCACTCTCGGTGTCCATT
TATATAAGATAAAACTTAAATAAACATCCACCACTCTCCCAAATACGCAG
GCGCACAAGGGGGCTCCGCCCCCTTAAACCCCCAAGGGGGCTCCGCCCCC
TTAAACCCCCAAGGGGGCTCCGCCCCCTTACACCCCCTAATAAATATTCA
ACAGGAAAACCACCTAATTAGAATTGCCGACCACAAACCGTCACTTACTT
CTCCTTTTTGCACTTACTTCCTCTTTTACTTATTATTATTCATTACATTA
ATTAATAATCACTGTAATTCCGGGGAGGAGCTAACAATCTATATAACTAA
CTACACTTCCGAATGGCTGAGTTTATGCCGCCAGACGGAGACGGGATCAC
TTCAGTGACTCCAGGCTGAACTTGGG (SEQ ID NO: 1023)
Annotations:

| Putative Domain | Base range |
|---|---|
| ORF1 | 283-2250 |
| ORF2 | 59-391 |
| ORF3 | 2277-2462 |
| GC-rich region, or a portion | 2515-2615 |
| thereof | |
| 5′ UTR Conserved Domain, or | 1-71 |
| a portion thereof | |

TABLE A24
Novel Anellovirus amino acid
sequence (Betatorquevirus)
RING 19 (Betatorquevirus)

| | |
|---|---|
| ORF1 | MPWYPRRRYPRRRYRWLRRWRARRPFRPRYRRRYWVRNYSRKR |
| | KLFKITTKEWQPKVIRKTHVKGTYPLFLCTKHRINNNMIQYLD |
| | SIAPEHYYGGGGFSIMQFSLQALYEEFIKAKNWWTNTNCFLPL |
| | VRYMGCSFKFYKTEFYDYIVLIERCYPLACTDEMYLSTQPSIM |
| | MLTRKCIFVPCKQNSKGKKPYKKVRVRPPSQMTTGWHFSQDLA |
| | NMPLVVLKTSVCSFDRYYTDSTAKSTTIGFKTLNTQTFRYHDW |
| | QEPPTTGYKPQNLLWFYGAENGSPVDPNNTIVSNLIYLGGTGP |
| | YEKGTPIKTNISNYFSEPKLWGNIFHDDYTSGTSPVFVINKSP |
| | SEIKTAWNTIKDLTVKASGVFTLRTIPLWLPCRYNPFADKATN |
| | NKIWLVSIHSDHTEWKPIDNPLLQRTDLPLWLLVWGWQDWQKK |
| | NQQTSQPDINYLTVISSPYISCYPKLDYYVLLDEGFWEGHSTY |
| | IESITDSDKKHWYPKNRFQIETLNLIANTGPGTVKLRENQAAE |
| | GHMVYRFNFKLGGCPAPMEKICDPSKQSKYPIPNNQQQTTSLQ |
| | SPENPIQTYLYDFDERRGLLTERATKRIKQDHTSEKTVLPFTG |
| | AATDLPILQTTSQEESSSEEEEEQQAEKKLLQLRRKQHRLRER |
| | ILQLLDIQNT (SEQ ID NO: 1024) |
| ORF2 | MAERARNLNIIILQMQIQPPIRTFKQTISDWKNLIVHVHDNIC |
| | NCNKPLEHTIDTCITNPDELRLNKSTKQQLQKCLGTPEEDTQE |
| | DVIDGFADGELDALFAQDTEEDTG (SEQ ID NO: 1025) |
| ORF3 | MFGDTHVPNRRMTPEEFEQELIVAGVFRRPPCYYIKDRPTYPY |
| | VPKPTDEKCMVNFDLNFP (SEQ ID NO: 1026) |

TABLE N25
Novel Anellovirus nucleic acid
sequence (Betatorquevirus)

| | |
|---|---|
| Name | RING 19 alternate |
| Genus/Clade | Betatorquevirus |
| Accession | N/A |

Full Sequence: 2876 bp
1 10 20 30 40 50
| | | | | |
CGGGAGCCGAAGGTGAGTGCAACCACCGTAGTCTAGGGGCAATTCGGGCT
AGTTCAGTATGGCGGAACGGGCAAGAAACTTAAATATTATTATTTTACAG
ATGCAAATACAACCACCTATTAGAACCTTCAAACAAACAATTTCAGATTG
GAAAAACTTAATTGTCCACGTTCACGACAACATTTGCAACTGCAATAAAC
CATTAGAACACACTATTGATACCTGTATCACCAATCCAGATGAATTAAGA
TTAAACAAATCTACTAAACAACAACTACAAAAATGCCTTGGTACCCCAGA
AGAAGATACCCAAGAAGACGTTATCGATGGCTTCGCAGATGGAGAGCTAG
ACGCCCTTTTCGCCCAAGATACAGAAGAAGATACTGGGTAAGAAACTATT
CTCGAAAGAGAAAACTATTTAAAATAACAACCAAAGAATGGCAACCAAAA
GTTATAAGAAAGACTCATGTAAAGGGCACCTATCCTTTGTTTCTTTGTAC
AAAGCACAGAATTAACAATAATATGATACAATATTTAGACTCTATAGCTC
CAGAACACTATTACGGAGGAGGAGGATTTTCAATAATGCAATTTTCCTTA
CAAGCCTTATATGAAGAATTTATAAAAGCAAAAAACTGGTGGACTAATAC
AAACTGCTTTTTACCACTTGTAAGATATATGGGTTGCTCATTCAAATTTT
ATAAAACTGAATTTTATGATTATATTGTACTAATTGAAAGATGTTATCCA
CTTGCTTGTACTGATGAAATGTACTTATCTACTCAACCTAGTATTATGAT
GCTTACAAGAAAATGTATTTTTGTACCATGCAAACAAAACAGCAAAGGTA
AAAAACCTTACAAAAAAGTTAGAGTAAGACCACCTTCACAAATGACTACA
GGATGGCATTTCTCACAAGACTTAGCAAACATGCCACTTGTAGTACTAAA
AACTTCAGTATGCAGCTTTGACAGATATTACACAGACAGTACAGCTAAAT
CAACCACAATAGGCTTTAAAACACTTAACACACAAACATTTAGATATCAT
GACTGGCAGGAACCACCTACAACAGGATACAAACCACAAAACCTACTATG
GTTTTATGGAGCAGAAAACGGATCACCAGTAGACCCCAACAACACAATAG
TATCAAACCTAATATACTTAGGAGGCACAGGACCTTATGAAAAAGGCACA
CCAATAAAAACAAACATAAGCAATTACTTTTCAGAGCCTAAACTGTGGGG
AAATATATTTCACGATGATTATACATCAGGAACATCACCCGTGTTTGTTA
CAAACAAATCACCATCAGAAATTAAAACCGCATGGAACACTATAAAAGAC
TTAACTGTTAAAGCTAGCGGTGTATTTACATTAAGAACAATTCCACTATG
GCTACCTTGCAGATACAACCCATTTGCAGACAAAGCAACCAACAACAAAA
TATGGCTAGTTTCTATACATTCAGACCACACAGAATGGAAACCAATAGAC
AATCCATTACTACAACGAACAGACCTTCCTTTATGGTTACTTGTATGGGG
TTGGCAAGATTGGCAGAAAAAAAACCAACAAACTTCACAACCTGATATTA
ATTATTTAACAGTAATATCTTCACCATATATATCATGCTACCCAAAATTA
GATTACTATGTGTTACTAGATGAAGGATTTTGGGAGGGTCACTCAACATA
CATAGAGTCAATTACAGACTCAGACAAAAAACACTGGTACCCTAAAAATA
GATTTCAAATAGAAACACTTAATCTAATAGCTAACACAGGTCCAGGAACT
GTAAAACTAAGAGAAAACCAAGCAGCAGAAGGTCACATGGTATATCGCTT
TAATTTTAAGCTTGGAGGATGTCCCGCACCGATGGAAAAAATATGTGACC
CTAGCAAACAATCCAAATATCCTATTCCCAATAACCAGCAACAAACAACT
TCGTTGCAGAGTCCAGAAAACCCAATTCAAACCTATCTCTACGACTTCGA
CGAAAGGAGGGGCCTACTTACAGAAAGAGCTACAAAAAGAATCAAACAAG
ATCACACATCTGAAAAAACTGTTTTGCCATTTACAGGAGCAGCAACAGAC
CTCCCCATACTCCAAACAACATCACAGGAGGAAAGCTCCTCGGAAGAAGA
AGAAGAGCAACAAGCGGAGAAGAAACTACTCCAGCTCCGAAGAAAGCAGC
ACCGACTCCGGGAGCGAATCCTCCAGCTATTAGACATACAAAATACATAA
TAAAACAAAGTACTGTAAAAATTGATATGTTTGGAGATACTCATGTACCT
AACCGTAGAATGACCCCAGAAGAATTTGAACAAGAACTAATTGTCGCTGG
TGTTTTTCGCAGACCTCCTTGTTACTATATAAAAGATAGACCTACTTATC
CTTATGTACCAAAACCTACTGATGAAAAATGTATGGTAAACTTTGACTTA
AACTTTCCTTAATAAACTACGCCTGCAAACTTTCACTCTCGGTGTCCATT
TATATAAGATAAAACTTAAATAAACATCCACCACTCTCCCAAATACGCAG
GCGCACAAGGGGGCTCCGCCCCCTTAAACCCCCAAGGGGGCTCCGCCCCC
TTAAACCCCCAAGGGGGCTCCGCCCCCTTACACCCCCTAATAAATATTCA
ACAGGAAAACCACCTAATTAGAATTGCCGACCACAAACCGTCACTTACTT
CTCCTTTTTGCACTTACTTCCTCTTTTACTTATTATTATTCATTACATTA
ATTAATAATCACTGTAATTCCGGGGAGGAGCTAACAATCTATATAACTAA
CTACACTTCCGAATGGCTGAGTTTATGCCGCCAGACGGAGACGGGATCAC
TTCAGTGACTCCAGGCTGAACTTGGG (SEQ ID NO: 1027)
Annotations:

| Putative Domain | Base range |
|---|---|
| ORF1 | 283-2250 |
| ORF2 | 101-391 |
| ORF3 | 2277-2462 |
| GC-rich region, or a | 2515-2615 |
| portion thereof | |
| 5′ UTR Conserved Domain, | 1-71 |
| or a portion thereof | |

TABLE A25
Novel Anellovirus amino acid
sequence (Betatorquevirus)
RING 19 (Betatorquevirus)

| | |
|---|---|
| ORF1 | MPWYPRRRYPRRRYRWLRRWRARRPFRPRYRRRYWVRNYSRKR |
| | KLFKITTKEWQPKVIRKTHVKGTYPLFLCTKHRINNNMIQYLD |
| | SIAPEHYYGGGGFSIMQFSLQALYEEFIKAKNWWTNTNCFLPL |
| | VRYMGCSFKFYKTEFYDYIVLIERCYPLACTDEMYLSTQPSIM |
| | MLTRKCIFVPCKQNSKGKKPYKKVRVRPPSQMTTGWHFSQDLA |
| | NMPLVVLKTSVCSFDRYYTDSTAKSTTIGFKTLNTQTFRYHDW |
| | QEPPTTGYKPQNLLWFYGAENGSPVDPNNTIVSNLIYLGGTGP |
| | YEKGTPIKTNISNYFSEPKLWGNIFHDDYTSGTSPVFVINKSP |
| | SEIKTAWNTIKDLTVKASGVFTLRTIPLWLPCRYNPFADKATN |
| | NKIWLVSIHSDHTEWKPIDNPLLQRTDLPLWLLVWGWQDWQKK |
| | NQQTSQPDINYLTVISSPYISCYPKLDYYVLLDEGFWEGHSTY |
| | IESITDSDKKHWYPKNRFQIETLNLIANTGPGTVKLRENQAAE |
| | GHMVYRFNFKLGGCPAPMEKICDPSKQSKYPIPNNQQQTTSLQ |
| | SPENPIQTYLYDFDERRGLLTERATKRIKQDHTSEKTVLPFTG |
| | AATDLPILQTTSQEESSSEEEEEQQAEKKLLQLRRKQHRLRER |
| | ILQLLDIQNT (SEQ ID NO: 1028) |
| ORF2 | MQIQPPIRTFKQTISDWKNLIVHVHDNICNCNKPLEHTIDTCI |
| | TNPDELRLNKSTKQQLQKCLGTPEEDTQEDVIDGFADGELDAL |
| | FAQDTEEDTG (SEQ ID NO: 1029) |
| ORF3 | MFGDTHVPNRRMTPEEFEQELIVAGVFRRPPCYYIKDRPTYPY |
| | VPKPTDEKCMVNFDLNFP (SEQ ID NO: 1030) |

In some embodiments, an anellovector or anelloVLP as described herein is a chimeric anellovector or anelloVLP. In some embodiments, a chimeric anellovector or anelloVLP further comprises one or more elements, polypeptides, or nucleic acids from a virus other than an Anellovirus.

In some embodiments, the chimeric anellovector or anelloVLP comprises a plurality of polypeptides (e.g., Anellovirus ORF1, ORF1/1, ORF1/2, ORF2, ORF2/2, ORF2/3, and/or ORF2t/3) comprising sequences from a plurality of different Anelloviruses (e.g., as described herein).

In some embodiments, the anellovector or anelloVLP comprises a chimeric polypeptide (e.g., Anellovirus ORF1, ORF1/1, ORF1/2, ORF2, ORF2/2, ORF2/3, and/or ORF2t/3), e.g., comprising at least one portion from an Anellovirus (e.g., as described herein) and at least one portion from a different virus (e.g., as described herein).

In some embodiments, the anellovector or anelloVLP comprises a chimeric polypeptide (e.g., Anellovirus ORF1, ORF1/1, ORF1/2, ORF2, ORF2/2, ORF2/3, and/or ORF2t/3), e.g., comprising at least one portion from one Anellovirus (e.g., as described herein) and at least one portion from a different Anellovirus (e.g., as described herein). In some embodiments, the anellovector or anelloVLP comprises a chimeric ORF1 molecule comprising at least one portion of an ORF1 molecule from one Anellovirus (e.g., as described herein), or an ORF1 molecule having at least 75%, 80%, 85%, 90%, 95%, 96%, 97%, 98%, or 99% amino acid sequence identity thereto, and at least one portion of an ORF1 molecule from a different Anellovirus (e.g., as described herein), or an ORF1 molecule having at least 75%, 80%, 85%, 90%, 95%, 96%, 97%, 98%, or 99% amino acid sequence identity thereto. In some embodiments, the chimeric ORF1 molecule comprises an ORF1 jelly-roll domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 arginine-rich region from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 hypervariable domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 N22 domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto. In some embodiments, the chimeric ORF1 molecule comprises an ORF1 C-terminal domain from one Anellovirus, or a sequence having at least 75%, 80%, 85%, 90%, 95%, 96%, 97%, 98%, or 99% sequence identity thereto, and an ORF1 amino acid subsequence (e.g., as described herein) from a different Anellovirus, or a sequence having at least 75%, 80%, 85%, 90%, 95%, 96%, 97%, 98%, or 99% sequence identity thereto.

In some embodiments, the anellovector or anelloVLP comprises a chimeric ORF1/1 molecule comprising at least one portion of an ORF1/1 molecule from one Anellovirus (e.g., as described herein), or an ORF1/1 molecule having at least 75%, 80%, 85%, 90%, 95%, 96%, 97%, 98%, or 99% amino acid sequence identity thereto, and at least one portion of an ORF1/1 molecule from a different Anellovirus (e.g., as described herein), or an ORF1/1 molecule having at least 75%, 80%, 85%, 90%, 95%, 96%, 97%, 98%, or 99% amino acid sequence identity thereto. In some embodiments, the anellovector or anelloVLP comprises a chimeric ORF1/2 molecule comprising at least one portion of an ORF1/2 molecule from one Anellovirus (e.g., as described herein), or an ORF1/2 molecule having at least 75%, 80%, 85%, 90%, 95%, 96%, 97%, 98%, or 99% amino acid sequence identity thereto, and at least one portion of an ORF1/2 molecule from a different Anellovirus (e.g., as described herein), or an ORF1/2 molecule having at least 75%, 80%, 85%, 90%, 95%, 96%, 97%, 98%, or 99% amino acid sequence identity thereto. In some embodiments, the anellovector or anelloVLP comprises a chimeric ORF2 molecule comprising at least one portion of an ORF2 molecule from one Anellovirus (e.g., as described herein), or an ORF2 molecule having at least 75%, 80%, 85%, 90%, 95%, 96%, 97%, 98%, or 99% amino acid sequence identity thereto, and at least one portion of an ORF2 molecule from a different Anellovirus (e.g., as described herein), or an ORF2 molecule having at least 75%, 80%, 85%, 90%, 95%, 96%, 97%, 98%, or 99% amino acid sequence identity thereto. In some embodiments, the anellovector or anelloVLP comprises a chimeric ORF2/2 molecule comprising at least one portion of an ORF2/2 molecule from one Anellovirus (e.g., as described herein), or an ORF2/2 molecule having at least 75%, 80%, 85%, 90%, 95%, 96%, 97%, 98%, or 99% amino acid sequence identity thereto, and at least one portion of an ORF2/2 molecule from a different Anellovirus (e.g., as described herein), or an ORF2/2 molecule having at least 75%, 80%, 85%, 90%, 95%, 96%, 97%, 98%, or 99% amino acid sequence identity thereto. In some embodiments, the anellovector or anelloVLP comprises a chimeric ORF2/3 molecule comprising at least one portion of an ORF2/3 molecule from one Anellovirus (e.g., as described herein), or an ORF2/3 molecule having at least 75%, 80%, 85%, 90%, 95%, 96%, 97%, 98%, or 99% amino acid sequence identity thereto, and at least one portion of an ORF2/3 molecule from a different Anellovirus (e.g., as described herein), or an ORF2/3 molecule having at least 75%, 80%, 85%, 90%, 95%, 96%, 97%, 98%, or 99% amino acid sequence identity thereto. In some embodiments, the anellovector or anelloVLP comprises a chimeric ORF2T/3 molecule comprising at least one portion of an ORF2T/3 molecule from one Anellovirus (e.g., as described herein), or an ORF2T/3 molecule having at least 75%, 80%, 85%, 90%, 95%, 96%, 97%, 98%, or 99% amino acid sequence identity thereto, and at least one portion of an ORF2T/3 molecule from a different Anellovirus (e.g., as described herein), or an ORF2T/3 molecule having at least 75%, 80%, 85%, 90%, 95%, 96%, 97%, 98%, or 99% amino acid sequence identity thereto.

In some embodiments, an anellovector comprises a nucleic acid comprising a sequence listed in PCT Application No. PCT/US2018/037379, incorporated herein by reference in its entirety. In some embodiments, an anellovector or anelloVLP comprises a polypeptide comprising a sequence listed in PCT Application No. PCT/US2018/037379, incorporated herein by reference in its entirety.

In some embodiments, an anellovector comprises an Anellovirus genome, e.g., as identified according to the method described in Example 9 of PCT Publication No. WO 2020/123816, incorporated by reference herein in its entirety. In some embodiments, an anellovector or anelloVLP comprises an Anellovirus sequence, or a portion thereof, as described in Example 30.

In some embodiments, an anellovector comprises a genetic element comprising a consensus Anellovirus motif, e.g., as shown in Table 19. In some embodiments, an anellovector comprises a genetic element comprising a consensus Anellovirus ORF1 motif, e.g., as shown in Table 19. In some embodiments, an anellovector comprises a genetic element comprising a consensus Anellovirus ORF1/1 motif, e.g., as shown in Table 19. In some embodiments, an anellovector comprises a genetic element comprising a consensus Anellovirus ORF1/2 motif, e.g., as shown in Table 19. In some embodiments, an anellovector comprises a genetic element comprising a consensus Anellovirus ORF2/2 motif, e.g., as shown in Table 19. In some embodiments, an anellovector comprises a genetic element comprising a consensus Anellovirus ORF2/3 motif, e.g., as shown in Table 19. In some embodiments, an anellovector comprises a genetic element comprising a consensus Anellovirus ORF2t/3 motif, e.g., as shown in Table

19. In some embodiments, X, as shown in Table 19, indicates any amino acid. In some embodiments, Z, as shown in Table 19, indicates glutamic acid or glutamine. In some embodiments, B, as shown in Table

19, indicates aspartic acid or asparagine. In some embodiments, J, as shown in Table 19, indicates leucine or isoleucine.

TABLE 19
Consensus motifs in open reading
frames (ORFs) of Anelloviruses

| | Open | | | SEQ |
|---|---|---|---|---|
| Consensus | Reading | | | ID |
| Threshold | Frame | Position | Motif | NO: |
| 50 | ORF1 | 79 | LIJRQWQPXXIRRCXIXG | 68 |
| | | | YXPLIXC | |
| 50 | ORF1 | 111 | NYXXHXD | 69 |
| 50 | ORF1 | 135 | FSLXXLYDZ | 70 |
| 50 | ORF1 | 149 | NXWTXSNXDLDLCRYXGC | 71 |
| 50 | ORF1 | 194 | TXPSXHPGXMXLXKHK | 72 |
| 50 | ORF1 | 212 | IPSLXTRPXG | 73 |
| 50 | ORF1 | 228 | RIXPPXLFXDKWYFQXDL | 74 |
| 50 | ORF1 | 250 | LLXIXATA | 75 |
| 50 | ORF1 | 260 | LXXPFXSPXTD | 76 |
| 50 | ORF1 | 448 | YNPXXDKGXGNXIW | 77 |
| 50 | ORF1 | 519 | CPYTZPXL | 78 |
| 50 | ORF1 | 542 | XFGXGXMP | 79 |
| 50 | ORF1 | 569 | HQXEVXEX | 80 |
| 50 | ORF1 | 600 | KYXFXFXWGGNP | 81 |
| 50 | ORF1 | 653 | HSWDXRRG | 82 |
| 50 | ORF1 | 666 | AIKRXQQ | 83 |
| 50 | ORF1 | 750 | XQZQXXLR | 84 |
| 50 | ORF1/1 | 73 | PRXJQXXDP | 85 |
| 50 | ORF1/1 | 91 | HSWDXRRG | 86 |
| 50 | ORF1/1 | 105 | AIKRXQQ | 87 |
| 50 | ORF1/1 | 187 | QZQXXLR | 88 |
| 50 | ORF1/2 | 97 | KXKRRRR | 89 |
| 50 | ORF2/2 | 158 | PIXSLXXYKXXTR | 90 |
| 50 | ORF2/2 | 189 | LAXQLLKECXKN | 91 |
| 50 | ORF2/3 | 39 | HLNXLA | 92 |
| 50 | ORF2/3 | 272 | DRPPR | 93 |
| 50 | ORF2/3 | 281 | DXPFYPWXP | 94 |
| 50 | ORF2/3 | 300 | VXFKLXF | 95 |
| 50 | ORF2t/3 | 4 | WXPPVHBVXGIERXW | 96 |
| 50 | ORF2t/3 | 37 | AKRKLX | 97 |
| 50 | ORF2t/3 | 140 | PSSXDWXXEY | 98 |
| 50 | ORF2t/3 | 156 | DRPPR | 99 |
| 50 | ORF2t/3 | 167 | PFYPW | 100 |
| 50 | ORF2t/3 | 183 | NVXFKLXF | 101 |
| 50 | ORF1 | 84 | JXXXXWQPXXXXXCXIXG | 102 |
| | | | XXXJWQP | |
| 50 | ORF1 | 149 | NXWXXXNXXXXLXRY | 103 |
| 50 | ORF1 | 448 | YNPXXDXG | 104 |

### ORF1 Molecules

In some embodiments, the anellovector or anelloVLP comprises an ORF1 molecule and/or a nucleic acid encoding an ORF1 molecule. Generally, an ORF1 molecule comprises a polypeptide having the structural features and/or activity of an Anellovirus ORF1 protein (e.g., an Anellovirus ORF1 protein as described herein, e.g., as listed in any one of Tables A1-A25), or a functional fragment thereof. In some embodiments, the ORF1 molecule comprises a truncation relative to an Anellovirus ORF1 protein (e.g., an Anellovirus ORF1 protein as described herein, e.g., as listed in any one of Tables A1-A25). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 70%, 75%, 80%, 85%, 90%, 95%, 96%, 97%, 98%, 99%, or 100% sequence identity to an Anellovirus ORF1 protein sequence as shown in any one of Tables A1-A25. In some embodiments, an ORF1 molecule comprises an amino acid sequence having at least 75%, 80%, 85%, 90%, 95%, 96%, 97%, 98%, or 99% sequence identity to an Betatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

In some embodiments, an ORF1 molecule as described herein comprises an amino acid sequence (e.g., an ORF1 sequence, or an arginine-rich region, jelly-roll domain, HVR, N22, or C-terminal domain sequence) as listed in any of Tables A2, A4, A6, A8, A10, A12, C₁-C₅, 2, 4, 6, 8, 10, 12, 14, 16, 18, 20-37, or D1-D10 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may encapsidate a nucleic acid molecule (e.g., a genetic element as described herein). In some embodiments, a plurality of ORF1 molecules may form a multimer, e.g., to produce a proteinaceous exterior. In some embodiments, the multimer may be a homomultimer. In other embodiments, the multimer may be a heteromultimer (e.g., comprising a plurality of distinct ORF1 molecules). It is also contemplated that an ORF1 molecule may have replicase activity.

An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands).

### Arginine-Rich Region

An arginine rich region has at least 70% (e.g., at least about 70, 80, 90, 95, 96, 97, 98, 99, or 100%) sequence identity to an arginine-rich region sequence described herein or a sequence of at least about 40 amino acids comprising at least 60%, 70%, or 80% basic residues (e.g., arginine, lysine, or a combination thereof).

### Jelly Roll Domain

A jelly-roll domain or region comprises (e.g., consists of) a polypeptide (e.g., a domain or region comprised in a larger polypeptide) comprising one or more (e.g., 1, 2, or 3) of the following characteristics:
(i) at least 30% (e.g., at least 30%, 35%, 40%, 45%, 50%, 55%, 60%, 65%, 70%, 75%, 80%, 90%, or more) of the amino acids of the jelly-roll domain are part of one or more β-sheets;
(ii) the secondary structure of the jelly-roll domain comprises at least four (e.g., at least 4, 5, 6, 7, 8, 9, 10, 11, or 12) β-strands; and/or
(iii) the tertiary structure of the jelly-roll domain comprises at least two (e.g., at least 2, 3, or 4) (3-sheets; and/or
(iv) the jelly-roll domain comprises a ratio of β-sheets to α-helices of at least 2:1, 3:1, 4:1, 5:1, 6:1, 7:1, 8:1, 9:1, or 10:1.

In certain embodiments, a jelly-roll domain comprises two β-sheets.

In certain embodiments, one or more (e.g., 1, 2, 3, 4, 5, 6, 7, 8, 9, or 10) of the β-sheets comprises about eight (e.g., 4, 5, 6, 7, 8, 9, 10, 11, or 12) β-strands. In certain embodiments, one or more (e.g., 1, 2, 3, 4, 5, 6, 7, 8, 9, or 10) of the β-sheets comprises eight β-strands. In certain embodiments, one or more (e.g., 1, 2, 3, 4, 5, 6, 7, 8, 9, or 10) of the β-sheets comprises seven β-strands. In certain embodiments, one or more (e.g., 1, 2, 3, 4, 5, 6, 7, 8, 9, or 10) of the β-sheets comprises six β-strands. In certain embodiments, one or more (e.g., 1, 2, 3, 4, 5, 6, 7, 8, 9, or 10) of the β-sheets comprises five β-strands. In certain embodiments, one or more (e.g., 1, 2, 3, 4, 5, 6, 7, 8, 9, or 10) of the β-sheets comprises four β-strands.

In some embodiments, the jelly-roll domain comprises a first β-sheet in antiparallel orientation to a second β-sheet. In certain embodiments, the first β-sheet comprises about four (e.g., 3, 4, 5, or 6) 0-strands. In certain embodiments, the second β-sheet comprises about four (e.g., 3, 4, 5, or 6) β-strands.

In embodiments, the first and second β-sheet comprise, in total, about eight (e.g., 6, 7, 8, 9, 10, 11, or 12) β-strands.

In certain embodiments, a jelly-roll domain is a component of a capsid protein (e.g., an ORF1 molecule as described herein). In certain embodiments, a jelly-roll domain has self-assembly activity. In some embodiments, a polypeptide comprising a jelly-roll domain binds to another copy of the polypeptide comprising the jelly-roll domain. In some embodiments, a jelly-roll domain of a first polypeptide binds to a jelly-roll domain of a second copy of the polypeptide.

An ORF1 molecule may also include a third region comprising the structure or activity of an Anellovirus N22 domain (e.g., as described herein, e.g., an N22 domain from an Anellovirus ORF1 protein as described herein), and/or a fourth region comprising the structure or activity of an Anellovirus C-terminal domain (CTD) (e.g., as described herein, e.g., a CTD from an Anellovirus ORF1 protein as described herein). In some embodiments, the ORF1 molecule comprises, in N-terminal to C-terminal order, the first, second, third, and fourth regions.

The ORF1 molecule may, in some embodiments, further comprise a hypervariable region (HVR), e.g., an HVR from an Anellovirus ORF1 protein, e.g., as described herein. In some embodiments, the HVR is positioned between the second region and the third region. In some embodiments, the HVR comprises comprises at least about 55 (e.g., at least about 45, 50, 51, 52, 53, 54, 55, 56, 57, 58, 59, 60, or 65) amino acids (e.g., about 45-160, 50-160, 55-160, 60-160, 45-150, 50-150, 55-150, 60-150, 45-140, 50-140, 55-140, or 60-140 amino acids).

In some embodiments, the first region can bind to a nucleic acid molecule (e.g., DNA). In some embodiments, the basic residues are selected from arginine, histidine, or lysine, or a combination thereof.

In some embodiments, the first region comprises at least 60%, 65%, 70%, 75%, 80%, 85%, 90%, 95%, or 100% arginine residues (e.g., between 60%-90%, 60%-80%, 70%-90%, or 70-80% arginine residues). In some embodiments, the first region comprises about 30-120 amino acids (e.g., about 40-120, 40-100, 40-90, 40-80, 40-70, 50-100, 50-90, 50-80, 50-70, 60-100, 60-90, or 60-80 amino acids). In some embodiments, the first region comprises the structure or activity of a viral ORF1 arginine-rich region (e.g., an arginine-rich region from an Anellovirus ORF1 protein, e.g., as described herein). In some embodiments, the first region comprises a nuclear localization sigal.

In some embodiments, the second region comprises a jelly-roll domain, e.g., the structure or activity of a viral ORF1 jelly-roll domain (e.g., a jelly-roll domain from an Anellovirus ORF1 protein, e.g., as described herein). In some embodiments, the second region is capable of binding to the second region of another ORF1 molecule, e.g., to form a proteinaceous exterior (e.g., capsid) or a portion thereof.

In some embodiments, the fourth region is exposed on the surface of a proteinaceous exterior (e.g., a proteinaceous exterior comprising a multimer of ORF1 molecules, e.g., as described herein).

In some embodiments, the first region, second region, third region, fourth region, and/or HVR each comprise fewer than four (e.g., 0, 1, 2, or 3) beta sheets.

In some embodiments, one or more of the first region, second region, third region, fourth region, and/or HVR may be replaced by a heterologous amino acid sequence (e.g., the corresponding region from a heterologous ORF1 molecule). In some embodiments, the heterologous amino acid sequence has a desired functionality, e.g., as described herein.

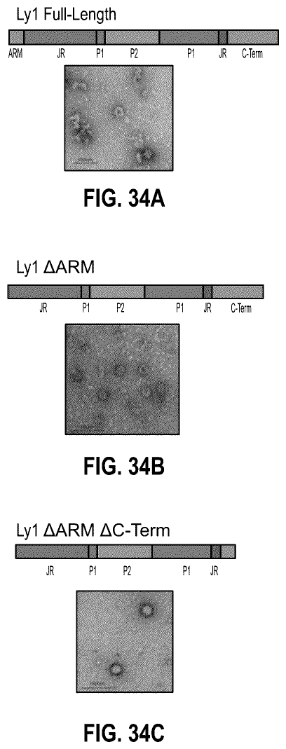

In some embodiments, the ORF1 molecule comprises a plurality of conserved motifs (e.g., motifs comprising about 5, 6, 7, 8, 9, 10, 11, 12, 13, 14, 15, 16, 17, 18, 19, 20, 25, 30, 35, 40, 45, 50, 60, 70, 80, 90, 100, or more amino acids) (e.g., as shown in FIG. 34 of PCT Publication No. WO2020/123816). In some embodiments, the conserved motifs may show 60, 70, 80, 85, 90, 95, or 100% sequence identity to an ORF1 protein of one or more wild-type Anellovirus clades (e.g., Betatorquevirus). In some embodiments, the conserved motifs each have a length between 1-1000 (e.g., between 5-10, 5-15, 5-20, 10-15, 10-20, 15-20, 5-50, 5-100, 10-50, 10-100, 10-1000, 50-100, 50-1000, or 100-1000) amino acids.

In certain embodiments, the conserved motifs consist of about 2-4% (e.g., about 1-8%, 1-6%, 1-5%, 1-4%, 2-8%, 2-6%, 2-5%, or 2-4%) of the sequence of the ORF1 molecule, and each show 100% sequence identity to the corresponding motifs in an ORF1 protein of the wild-type Anellovirus clade. In certain embodiments, the conserved motifs consist of about 5-10% (e.g., about 1-20%, 1-10%, 5-20%, or 5-10%) of the sequence of the ORF1 molecule, and each show 80% sequence identity to the corresponding motifs in an ORF1 protein of the wild-type Anellovirus clade. In certain embodiments, the conserved motifs consist of about 10-50% (e.g., about 10-20%, 10-30%, 10-40%, 10-50%, 20-40%, 20-50%, or 30-50%) of the sequence of the ORF1 molecule, and each show 60% sequence identity to the corresponding motifs in an ORF1 protein of the wild-type Anellovirus clade. In some embodiments, the conserved motifs comprise one or more amino acid sequences as listed in Table 19.

In some embodiments, an ORF1 molecule comprises at least one difference (e.g., a mutation, chemical modification, or epigenetic alteration) relative to a wild-type ORF1 protein, e.g., as described herein (e.g., as shown in any one of Tables A1-A25).

### Conserved ORF1 Motif in N22 Domain

In some embodiments, a polypeptide (e.g., an ORF1 molecule) described herein comprises the amino acid sequence YNPX²DXGX²N (SEQ ID NO: 829), wherein X” is a contiguous sequence of any n amino acids. For example, X² indicates a contiguous sequence of any two amino acids. In some embodiments, the YNPX²DXGX²N (SEQ ID NO: 829) is comprised within the N22 domain of an ORF1 molecule, e.g., as described herein. In some embodiments, a genetic element described herein comprises a nucleic acid sequence (e.g., a nucleic acid sequence encoding an ORF1 molecule, e.g., as described herein) encoding the amino acid sequence YNPX²DXGX²N (SEQ ID NO: 829), wherein X” is a contiguous sequence of any n amino acids.

In some embodiments, a polypeptide (e.g., an ORF1 molecule) comprises a conserved secondary structure, e.g., flanking and/or comprising a portion of the YNPX²DXGX²N (SEQ ID NO: 829) motif, e.g., in an N22 domain. In some embodiments, the conserved secondary structure comprises a first beta strand and/or a second beta strand. In some embodiments, the first beta strand is about 5-6 (e.g., 3, 4, 5, 6, 7, or 8) amino acids in length. In some embodiments, the first beta strand comprises the tyrosine (Y) residue at the N-terminal end of the YNPX²DXGX²N (SEQ ID NO: 829) motif. In some embodiments, the YNPX²DXGX²N (SEQ ID NO: 829) motif comprises a random coil (e.g., about 8-9 amino acids of random coil). In some embodiments, the second beta strand is about 7-8 (e.g., 5, 6, 7, 8, 9, or 10) amino acids in length. In some embodiments, the second beta strand comprises the asparagine (N) residue at the C-terminal end of the YNPX²DXGX²N (SEQ ID NO: 829) motif.

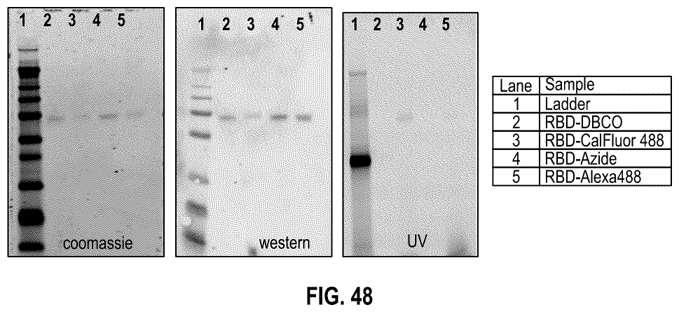

Exemplary YNPX²DXGX²N (SEQ ID NO: 829) motiβ-flanking secondary structures are described in Example 47 and FIG. 48 of PCT Publication No. WO 2020/123816, incorporated by reference herein in its entirety. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT Publication No. WO 2020/123816. In some embodiments, an ORF1 molecule comprises a region comprising one or more (e.g., 1, 2, 3, 4, 5, 6, 7, 8, 9, 10, or all) of the secondary structural elements (e.g., beta strands) shown in FIG. 48 of PCT Publication No. WO

2020/123816, incorporated by reference herein in its entirety, flanking a YNPX²DXGX²N (SEQ ID NO: 829) motif (e.g., as described herein).

### Conserved Secondary Structural Motif in ORF1 Jelly-Roll Domain

In some embodiments, a polypeptide (e.g., an ORF1 molecule) described herein comprises one or more secondary structural elements comprised by an Anellovirus ORF1 protein (e.g., as described herein). In some emboiments, an ORF1 molecule comprises one or more secondary structural elements comprised by the jelly-roll domain of an Anellovius ORF1 protein (e.g., as described herein). Generally, an ORF1 jelly-roll domain comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and a ninth beta strand. In some embodiments, an ORF1 molecule comprises a secondary structure comprising, in order in the N-terminal to C-terminal direction, a first beta strand, a second beta strand, a first alpha helix, a third beta strand, a fourth beta strand, a fifth beta strand, a second alpha helix, a sixth beta strand, a seventh beta strand, an eighth beta strand, and/or a ninth beta strand.

In some embodiments, a pair of the conserved secondary structural elements (i.e., the beta strands and/or alpha helices) are separated by an interstitial amino acid sequence, e.g., comprising a random coil sequence, a beta strand, or an alpha helix, or a combination thereof. Interstitial amino acid sequences between the conserved secondary structural elements may comprise, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more amino acids. In some embodiments, an ORF1 molecule may further comprise one or more additional beta strands and/or alpha helices (e.g., in the jelly-roll domain). In some embodiments, consecutive beta strands or consecutive alpha helices may be combined. In some embodiments, the first beta strand and the second beta strand are comprised in a larger beta strand. In some embodiments, the third beta strand and the fourth beta strand are comprised in a larger beta strand. In some embodiments, the fourth beta strand and the fifth beta strand are comprised in a larger beta strand. In some embodiments, the sixth beta strand and the seventh beta strand are comprised in a larger beta strand. In some embodiments, the seventh beta strand and the eighth beta strand are comprised in a larger beta strand. In some embodiments, the eighth beta strand and the ninth beta strand are comprised in a larger beta strand.

In some embodiments, the first beta strand is about 5-7 (e.g., 3, 4, 5, 6, 7, 8, 9, or 10) amino acids in length. In some embodiments, the second beta strand is about 15-16 (e.g., 13, 14, 15, 16, 17, 18, or 19) amino acids in length. In some embodiments, the first alpha helix is about 15-17 (e.g., 13, 14, 15, 16, 17, 18, 19, or 20) amino acids in length. In some embodiments, the third beta strand is about 3-4 (e.g., 1, 2 , 3, 4, 5, or 6) amino acids in length. In some embodiments, the fourth beta strand is about 10-11 (e.g., 8, 9, 10, 11, 12, or 13) amino acids in length. In some embodiments, the fifth beta strand is about 6-7 (e.g., 4, 5, 6, 7, 8, 9, or 10) amino acids in length. In some embodiments, the second alpha helix is about 8-14 (e.g., 5, 6, 7, 8, 9, 10, 11, 12, 13, 14, 15, 16, or 17) amino acids in length. In some embodiments, the second alpha helix may be broken up into two smaller alpha helices (e.g., separated by a random coil sequence). In some embodiments, each of the two smaller alpha helices are about 4-6 (e.g., 2, 3, 4, 5, 6, 7, or 8) amino acids in length. In some embodiments, the sixth beta strand is about 4-5 (e.g., 2, 3, 4, 5, 6, or 7) amino acids in length. In some embodiments, the seventh beta strand is about 5-6 (e.g., 3, 4, 5, 6, 7, 8, or 9) amino acids in length. In some embodiments, the eighth beta strand is about 7-9 (e.g., 5, 6, 7, 8, 9, 10, 11, 12, or 13) amino acids in length. In some embodiments, the ninth beta strand is about 5-7 (e.g., 3, 4, 5, 6, 7, 8, 9, or 10) amino acids in length.

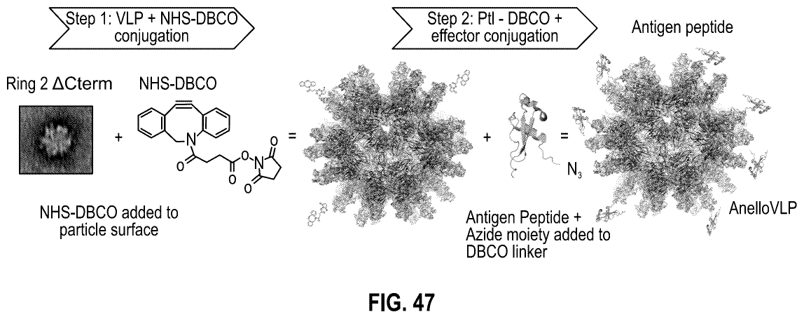

Exemplary jelly-roll domain secondary structures are described in Example 47 and FIG. 47 of PCT Publication No. WO 2020/123816, incorporated by reference herein in its entirety. In some embodiments, an ORF1 molecule comprises a region comprising one or more (e.g., 1, 2, 3, 4, 5, 6, 7, 8, 9, 10, or all) of the secondary structural elements (e.g., beta strands and/or alpha helices) of any of the jelly-roll domain secondary structures shown in FIG. 47 of PCT Publication No. WO 2020/123816, incorporated by reference herein in its entirety.

### Exemplary ORF1 Sequences

In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described herein). In some embodiments, an anellovector or anelloVLP described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described herein. In some embodiments, an anellovector or anelloVLP described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described herein.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) (e.g., as listed herein), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses. In some embodiments, the ORF1 molecule comprises one or more of an Arg-rich domain, a jelly-roll domain, an N22 domain, and a CTD from one Anellovirus, and an HVR from another. In some embodiments, the ORF1 molecule comprises one or more of a jelly-roll domain, an HVR, an N22 domain, and a CTD from one Anellovirus, and an Arg-rich domain from another. In some embodiments, the ORF1 molecule comprises one or more of an Arg-rich domain, an HVR, an N22 domain, and a CTD from one Anellovirus, and a jelly-roll domain from another. In some embodiments, the ORF1 molecule comprises one or more of an Arg-rich domain, a jelly-roll domain, an HVR, and a CTD from one Anellovirus, and an N22 domain from another. In some embodiments, the ORF1 molecule comprises one or more of an Arg-rich domain, a jelly-roll domain, an HVR, and an N22 domain from one Anellovirus, and a CTD from another.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) as described in PCT Publication No. WO2020/123816 (incorporated herein by reference in entirety).

In some embodiments, a polypeptide (e.g., an ORF1 molecule) described herein comprises an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables 20-37, or D1-D11). In some embodiments, an anellosome described herein comprises an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables 20-37, or D1-D11. In some embodiments, an anellosome described herein comprises a nucleic acid molecule (e.g., a genetic element) encoding an ORF1 molecule comprising an amino acid sequence having at least about 70%, 75%, 80%, 85%, 90%, 95%, 96%, 97%, 98%, 99%, or 100% sequence identity to one or more Anellovirus ORF1 subsequences, e.g., as described in any of Tables 20-37, or D1-D11.

In some embodiments, the one or more Anellovirus ORF1 subsequences comprises one or more of an arginine (Arg)-rich domain, a jelly-roll domain, a hypervariable region (HVR), an N22 domain, or a C-terminal domain (CTD) (e.g., as listed in any of Tables 20-37, or D1-D11), or sequences having at least about 70%, 75%, 80%, 85%, 90%, 95%, 96%, 97%, 98%, 99%, or 100% sequence identity thereto. In some embodiments, the ORF1 molecule comprises a plurality of subsequences from different Anelloviruses (e.g., any combination of ORF1 subsequences selected from the Alphatorquevirus Clade 1-7 subsequences listed in Tables 20-37, or D1-D11). In embodiments, the ORF1 molecule comprises one or more of an Arg-rich domain, a jelly-roll domain, an N22 domain, and a CTD from one Anellovirus, and an HVR from another. In embodiments, the ORF1 molecule comprises one or more of a jelly-roll domain, an HVR, an N22 domain, and a CTD from one Anellovirus, and an Arg-rich domain from another. In embodiments, the ORF1 molecule comprises one or more of an Arg-rich domain, an HVR, an N22 domain, and a CTD from one Anellovirus, and a jelly-roll domain from another. In embodiments, the ORF1 molecule comprises one or more of an Arg-rich domain, a jelly-roll domain, an HVR, and a CTD from one Anellovirus, and an N22 domain from another. In embodiments, the ORF1 molecule comprises one or more of an Arg-rich domain, a jelly-roll domain, an HVR, and an N22 domain from one Anellovirus, and a CTD from another.

TABLE 20
Exemplary Anellovirus ORF1 amino acid
subsequence (Alphatorquevirus, Clade 1)

| | |
|---|---|
| Name | CT30F |
| Genus/Clade | Alphatorquevirus, Clade 1 |
| Strain Accesion Number | AB064597.1 |
| Protein Accession | ANQ39351.1 |
| Number | |

Full Sequence: 680 AA
1 10 20 30 40 50
| | | | | |
TAWWWGRWRRRWRRRRPWRPRLRRRRARRAFPRRRRRRFVSRRWRRPYRR
RRRRGRRRRRRRRRHKPTLVLRQWQPDVIRHCKITGRMPLIICGKGSTQF
NYITHADDITPRGASYGGNFTNMTFSLEAIYEQFLYHRNRWSASNHDLEL
CRYKGTTLKLYRHPDVDYIVTYSRTGPFEISHMTYLSTHPLLMLLNKHHI
VVPSLKTKPRGRKAIKVRIRPPKLMNNKWYFTRDFCNIGLFQLWATGLEL
RNPWLRMSTLSPCIGENVLKNSIYTNLSNLPQHREDRLNIINNTLHPHDI
TGPNNKKWQYTYTKLMAPIYYSANRASTYDLLREYGLYSPYYLNPTRINL
DWMTPYTHVRYNPLVDKGFGNRIYIQWCSEADVSYNRTKSKCLLQDMPLF
FMCYGYIDWAIKNTGVSSLARDARICIRCPYTEPQLVGSTEDIGFVPITE
TFMRGDMPVLAPYIPLSWFCKWYPNIAHQKEVLEAIISCSPFMPRDQGMN
GWDITIGYKMDFLWGGSPLPSQPIDDPCQQGTHPIPDPDKHPRLLQVSNP
KLLGPRTVFHKWDIRRGQFSKRSIKRVSEYSSDDESLAPGLPSKRNKLDS
AFRGENPEQKECYSLLKALEEEETPEEEEPAPQEKAQKEELLHQLQLQRR
HQRVLRRGLKLVFTDILRLRQGVHWNPELT (SEQ ID NO: 173)
Annotations:

| Putative Domain | AA range |
|---|---|
| Arg-Rich Region | 1-66 |
| Jelly-roll domain | 67-277 |
| Hypervariable Region | 278-347 |
| N22 | 348-513 |
| C-terminal Domain | 513-680 |

TABLE 21
Exemplary Anellovirus ORF1 amino acid
subsequence (Alphatorquevirus, Clade 1)
TTV-CT30F-ORF1 (Alphatorquevirus Clade 1)

| | |
|---|---|
| Arg-Rich | TAWWWGRWRRRWRRRRPWRPRLRRRRARRAFPRRRRRRF |
| Region | VSRRWRRPYRRRRRRGRRRRRRRRRHK (SEQ ID NO: |
| | 174) |
| Jelly- | PTLVLRQWQPDVIRHCKITGRMPLIICGKGSTQFNYITH |
| roll | ADDITPRGASYGGNFTNMTFSLEAIYEQFLYHRNRWSAS |
| Domain | NHDLELCRYKGTTLKLYRHPDVDYIVTYSRTGPFEISHM |
| | TYLSTHPLLMLLNKHHIVVPSLKTKPRGRKAIKVRIRPP |
| | KLMNNKWYFTRDFCNIGLFQLWATGLELRNPWLRMSTLS |
| | PCIGFNVLKNSIYTNL (SEQ ID NO: 175) |
| Hyper- | SNLPQHREDRLNIINNTLHPHDITGPNNKKWQYTYTKLM |
| variable | APIYYSANRASTYDLLREYGLYSPYYLNPTR (SEQ ID |
| domain | NO: 176) |
| N22 | INLDWMTPYTHVRYNPLVDKGFGNRIYIQWCSEADVSYN |
| | RTKSKCLLQDMPLFFMCYGYIDWAIKNTGVSSLARDARI |
| | CIRCPYTEPQLVGSTEDIGFVPITETFMRGDMPVLAPYI |
| | PLSWFCKWYPNIAHQKEVLEAIISCSPFMPRDQGMNGWD |
| | ITIGYKMDFL (SEQ ID NO: 177) |
| C- | WGGSPLPSQPIDDPCQQGTHPIPDPDKHPRLLQVSNPKL |
| terminal | LGPRTVFHKWDIRRGQFSKRSIKRVSEYSSDDESLAPGL |
| domain | PSKRNKLDSAFRGENPEQKECYSLLKALEEEETPEEEEP |
| | APQEKAQKEELLHQLQLQRRHQRVLRRGLKLVFTDILRL |
| | RQGVHWNPELT (SEQ ID NO: 178) |

TABLE 22
Exemplary Anellovirus ORF1 amino acid
subsequence (Alphatorquevirus, Clade 2)

| | |
|---|---|
| Name | TTV-P13-1 |
| Genus/Clade | Alphatorquevirus, Clade 2 |
| Accession Number | KT163896.1 |
| Protein Accession | ANQ39351.1 |
| Number | |

Full Sequence: 747 AA
1 10 20 30 40 50
| | | | | |
MAYWWGRRRRWRRWRRRRRPLRRRRRWRRRRRWPRRRRWRRRRRRARPAR
RYRRRRGRRRVRRRRRPQKLVLTQWNPQTVRKCVIRGFLPLFFCGQGAYH
RNFTDHYDDVFPKGPSGGGHGSMVENLSFLYQEFKKHHNKWSRSNLDEDL
VRYKGTVIKLYRHQDFDYIVWISRTPPFQESLLTVMTHQPSVMLQAKKCI
IVKSYRTHPGGKPYVTAKVRPPRLLTDKWYFQSDFCNVPLFSLQFALAEL
RFPICSPQTDTNCINFLVLDDIYYKFLDNKPKQSSDPNDENRIKFWHGLW
STMRYLNTTYINTLFPGTDSLVAAKDTDNSVNKYPSTATKQPYKDSQYMQ
NIWNTSKIHALYTWVAETNYKRLQAYYTQTYGGYQRQFFTGKQYWDYRVG
MFSPAFLSPSRLNPQNPGAYTEVSYNPWTDEGTGNVVCLQYLTKETSDYK
PGGGSKFCIEGVPLWAALVGYVDMCKKEGKDPGIRLNCLLLVKCPYTKPQ
LYDKKNPEKLFVPYSYNFGHGKMPGGDKYIPIEFKDRWYPCLLHQEEWIE
DIVRSGPFVPKDMPSSVTCMMRYSSLFNWGGNIIQEQAVEDPCKKGTFVV
PGTSGIARILQVSNPAKQTPTTTWHSWDWRRSLFTETGLKRMREQQPYDE
LSYTGPKKPKLSLPAGPAVPGAAVASSWWETKQVTSPDVSETETEAEAHQ
EEETEPEEGVQLQQLWEQQLLQKRQLGVVFQQLLRLRQGAEIHPGLV
(SEQ ID NO: 179)
Annotations:

| Putative Domain | AA range |
|---|---|
| Arg-Rich Region | 1-69 |
| Jelly-roll domain | 70-279 |
| Hypervariable Region | 280-411 |
| N22 | 412-578 |
| C-terminal Domain | 579-747 |

TABLE 23
Exemplary Anellovirus ORF1 amino acid
subsequence (Alphatorquevirus, Clade 2)
TTV-P13-1-ORF1 (Alphatorquevirus Clade 2)

| | |
|---|---|
| Arg-Rich | MAYWWGRRRRWRRWRRRRRPLRRRRRWRRRRRWPRRRRW |
| Region | RRRRRRARPARRYRRRRGRRRVRRRRRPQK (SEQ ID |
| | NO: 180) |
| Jelly- | LVLTQWNPQTVRKCVIRGFLPLFFCGQGAYHRNFTDHYD |
| roll | DVFPKGPSGGGHGSMVFNLSFLYQEFKKHHNKWSRSNLD |
| Domain | FDLVRYKGTVIKLYRHQDFDYIVWISRTPPFQESLLTVM |
| | THQPSVMLQAKKCIIVKSYRTHPGGKPYVTAKVRPPRLL |
| | TDKWYFQSDFCNVPLFSLQFALAELRFPICSPQTDTNCI |
| | NFLVLDDIYYKFLDN (SEQ ID NO: 181) |
| Hyper- | KPKQSSDPNDENRIKFWHGLWSTMRYLNTTYINTLFPGT |
| variable | DSLVAAKDTDNSVNKYPSTATKQPYKDSQYMQNIWNTSK |
| domain | IHALYTWVAETNYKRLQAYYTQTYGGYQRQFFTGKQYWD |
| | YRVGMFSPAFLSPSR (SEQ ID NO:182) |
| N22 | LNPQNPGAYTEVSYNPWTDEGTGNVVCLQYLTKETSDYK |
| | PGGGSKFCIEGVPLWAALVGYVDMCKKEGKDPGIRLNCL |
| | LLVKCPYTKPQLYDKKNPEKLFVPYSYNFGHGKMPGGDK |
| | YIPIEFKDRWYPCLLHQEEWIEDIVRSGPFVPKDMPSSV |
| | TCMMRYSSLFN (SEQ ID NO: 183) |
| C- | WGGNIIQEQAVEDPCKKGTFVVPGTSGIARILQVSNPAK |
| terminal | QTPTTTWHSWDWRRSLFTETGLKRMREQQPYDELSYTGP |
| domain | KKPKLSLPAGPAVPGAAVASSWWETKQVTSPDVSETETE |
| | AEAHQEEETEPEEGVQLQQLWEQQLLQKRQLGVVFQQLL |
| | RLRQGAEIHPGLV (SEQ ID NO: 184) |

TABLE 24
Exemplary Anellovirus ORF1 amino acid
subsequence (Alphatorquevirus, Clade 3)

| | |
|---|---|
| Name | Ring1 |
| Genus/Clade | Alphatorquevirus, Clade 3 |
| Accession Number | AJ620231.1 |
| Protein Accession Number | CAF05750.1 |

Full Sequence: 743 AA
1 10 20 30 40 50
| | | | | |
MAWGWWKRRRRWWFRKRWTRGRLRRRWPRSARRRPRRRRVRRRRRWRRGR
RKTRTYRRRRRFRRRGRKAKLIIKLWQPAVIKRCRIKGYIPLIISGNGTF
ATNFTSHINDRIMKGPFGGGHSTMRFSLYILFEEHLRHMNFWTRSNDNLE
LTRYLGASVKIYRHPDQDFIVIYNRRTPLGGNIYTAPSLHPGNAILAKHK
ILVPSLQTRPKGRKAIRLRIAPPTLFTDKWYFQKDIADLTLFNIMAVEAD
LRFPFCSPQTDNTCISFQVLSSVYNNYLSINTENNDNSDSKLKEFLNKAF
PTTGTKGTSLNALNTFRTEGCISHPQLKKPNPQINKPLESQYFAPLDALW
GDPIYYNDLNENKSLNDIIEKILIKNMITYHAKLREFPNSYQGNKAFCHL
TGIYSPPYLNQGRISPEIFGLYTEIIYNPYTDKGTGNKVWMDPLTKENNI
YKEGQSKCLLTDMPLWTLLFGYTDWCKKDTNNWDLPLNYRLVLICPYTFP
KLYNEKVKDYGYIPYSYKFGAGQMPDGSNYIPFQFRAKWYPTVLHQQQVM
EDISRSGPFAPKVEKPSTQLVMKYCFNFNWGGNPIIEQIVKDPSFQPTYE
IPGTGNIPRRIQVIDPRVLGPHYSFRSWDMRRHTFSRASIKRVSEQQETS
DLVFSGPKKPRVDIPKQETQEESSHSLQRESRPWETEEESETEALSQESQ
EVPFQQQLQQQYQEQLKLRQGIKVLFEQLIRTQQGVHVNPCLR (SEQ
ID NO: 185)
Annotations:

| Putative Domain | AA range |
|---|---|
| Arg-Rich Region | 1-68 |
| Jelly-roll domain | 69-280 |
| Hypervariable Region | 281-413 |
| N22 | 414-579 |
| C-terminal Domain | 580-743 |

TABLE 25
Exemplary Anellovirus ORF1 amino acid
subsequence (Alphatorquevirus, Clade 3)
Ring1 ORF1 (Alphatorquevirus Clade 3)

| | |
|---|---|
| Arg-Rich | MAWGWWKRRRRWWFRKRWTRGRLRRRWPRSARRRPRRR |
| Region | RVRRRRRWRRGRRKTRTYRRRRRFRRRGRK (SEQ ID |
| | NO: 186) |
| Jelly- | AKLIIKLWQPAVIKRCRIKGYIPLIISGNGTFATNFTS |
| roll | HINDRIMKGPFGGGHSTMRFSLYILFEEHLRHMNFWTR |
| Domain | SNDNLELTRYLGASVKIYRHPDQDFIVIYNRRTPLGGN |
| | IYTAPSLHPGNAILAKHKILVPSLQTRPKGRKAIRLRI |
| | APPTLFTDKWYFQKDIADLTLFNIMAVEADLRFPFCSP |
| | QTDNTCISFQVLSSVYNNYLSI (SEQ ID NO: 187) |
| Hyper- | NTFNNDNSDSKLKEFLNKAFPTTGTKGTSLNALNTFRT |
| variable | EGCISHPQLKKPNPQINKPLESQYFAPLDALWGDPIYY |
| domain | NDLNENKSLNDIIEKILIKNMITYHAKLREFPNSYQGN |
| | KAFCHLTGIYSPPYLNQGR (SEQ ID NO: 188) |
| N22 | ISPEIFGLYTEIIYNPYTDKGTGNKVWMDPLTKENNIY |
| | KEGQSKCLLTDMPLWTLLFGYTDWCKKDTNNWDLPLNY |
| | RLVLICPYTFPKLYNEKVKDYGYIPYSYKFGAGQMPDG |
| | SNYIPFQFRAKWYPTVLHQQQVMEDISRSGPFAPKVEK |
| | PSTQLVMKYCFNFN (SEQ ID NO: 189) |
| C- | WGGNPIEQIVKDPSFQPTYEIPGTGNIPRRIQVIDPRV |
| terminal | LGPHYSFRSWDMRRHTFSRASIKRVSEQQETSDLVFSG |
| domain | PKKPRVDIPKQETQEESSHSLQRESRPWETEEESETEA |
| | LSQESQEVPFQQQLQQQYQEQLKLRQGIKVLFEQLIRT |
| | QQGVHVNPCLR (SEQ ID NO: 190) |

TABLE 26
Exemplary Anellovirus ORF1 amino acid
subsequence (Alphatorquevirus, Clade 4)

| | |
|---|---|
| Name | TTV-HD20a |
| Genus/Clade | Alphatorquevirus, Clade 4 |
| Accession Number | FR751492.1 |
| Protein Accession Number | NA |

Full Sequence: 780 AA
1 10 20 30 40 50
| | | | | |
MAWWGWRRRWWRPKRRWRWRRARRRRRVPARRPRRAFRRYRTRTVRRRRR
GRRRGYRRRYRLRRYARRRFRRKKIVLTQWNPQTTRKCIIRGMMPVLWAG
MGTGGRNYAVRSDDYVVNKGFGGSFATETFSLKVLYDQFORGENRWSHIN
EDLDLARYRGCRWTFYRHKDTDFIVYFTNNPPMKTNQFSAPLTTPGMLMR
SKYKVLIPSFQTRPKGRKTVTVKIRPPKLFQDKWYTQQDLCSVPLVQLNV
TAADFTHPFGSPLTETPCVEFQVLGDLYNTCLNIDLPQFSELGEITSAYS
KPNSNNLKELYKELFTKATSGHYWQTFITNSMVRAHIDADKAKEAQRAST
TPSYNNDPFPTIPVKSEFAQWKKKFTDTRDSPFLFATYHPEAIKDTIMKM
RENNFKLETGPNDKYGDYTAQYQGNTHMLDYYLGFYSPIFLSDGRSNVEF
FTAYRDIVYNPFLDKAQGNMVWFQYHTKTDNKFKKPECHWEIKDMPLWAL
LNGYVDYLETQIQYGDLSKEGKVLIRCPYTKPALVDPRDDTAGYVVYNRN
FGRGKWIDGGGYIPLHERTKWYVMLRYQTDVFHDIVTCGPWQYRDDNKNS
QLVAKYRFSFIWGGNTVHSQVIRNPCKDNQVSGPRRQPRDIQVVDPQRIT
PPWVLHSFDQRRGLFTETALRRLLQEPLPGEYAVSTLRTPLLFLPSEYQR
EDGAAESASGSPAKRPRIWSEESQTETISSEENPAETTRELLQRKLREQR
ALQFQLQHFAVQLAKTQANLHVNPLLSFPQ (SEQ ID NO: 191)
Annotations:

| Putative Domain | AA range |
|---|---|
| Arg-Rich Region | 1-74 |
| Jelly-roll domain | 75-284 |
| Hypervariable Region | 285-445 |
| N22 | 446-611 |
| C-terminal Domain | 612-780 |

TABLE 27
Exemplary Anellovirus ORF1 amino acid
subsequence (Alphatorquevirus, Clade 4)
TTV-HD20a-ORF1 (Alphatorquevirus Clade 4)

| | |
|---|---|
| Arg-Rich | MAWWGWRRRWWRPKRRWRWRRARRRRRVPARRPRRAFRR |
| Region | YRTRTVRRRRRGRRRGYRRRYRLRRYARRRFRRKK |
| | (SEQ ID NO: 192) |
| Jelly- | IVLTQWNPQTTRKCIIRGMMPVLWAGMGTGGRNYAVRSD |
| roll | DYVVNKGFGGSFATETFSLKVLYDQFQRGFNRWSHTNED |
| Domain | LDLARYRGCRWTFYRHKDTDFIVYFTNNPPMKTNQFSAP |
| | LTTPGMLMRSKYKVLIPSFQTRPKGRKTVTVKIRPPKLF |
| | QDKWYTQQDLCSVPLVQLNVTAADFTHPFGSPLTETPCV |
| | EFQVLGDLYNTCLNI (SEQ ID NO: 193) |
| Hyper- | DLPQFSELGEITSAYSKPNSNNLKELYKELFTKATSGHY |
| variable | WQTFITNSMVRAHIDADKAKEAQRASTTPSYNNDPFPTI |
| domain | PVKSEFAQWKKKFTDTRDSPFLFATYHPEAIKDTIMKMR |
| | ENNFKLETGPNDKYGDYTAQYQGNTHMLDYYLGFYSPIF |
| | LSDGR (SEQ ID NO: 194) |
| N22 | SNVEFFTAYRDIVYNPFLDKAQGNMVWFQYHTKTDNKFK |
| | KPECHWEIKDMPLWALLNGYVDYLETQIQYGDLSKEGKV |
| | LIRCPYTKPALVDPRDDTAGYVVYNRNFGRGKWIDGGGY |
| | IPLHERTKWYVMLRYQTDVFHDIVTCGPWQYRDDNKNSQ |
| | LVAKYRFSFI (SEQ ID NO: 195) |
| C- | WGGNTVHSQVIRNPCKDNQVSGPRRQPRDIQVVDPQRIT |
| terminal | PPWVLHSFDQRRGLFTETALRRLLQEPLPGEYAVSTLRT |
| domain | PLLFLPSEYQREDGAAESASGSPAKRPRIWSEESQTETI |
| | SSEENPAETTRELLQRKLREQRALQFQLQHFAVQLAKTQ |
| | ANLHVNPLLSFPQ (SEQ ID NO: 196) |

TABLE 28
Exemplary Anellovirus ORF1 amino acid
subsequence (Alphatorquevirus, Clade 5)

| | |
|---|---|
| Name | TTV-16 (TUS01) |
| Genus/Clade | Alphatorquevirus, Clade 5 |
| Accession Number | AB017613.1 |
| Protein Accession Number | BAA82454.1 |

Full Sequence: 761 AA
1 10 20 30 40 50
| | | | | |
MAYWFRRWGWRPRRRWRRWRRRRRRLPRRRTRRAVRGLGRRRKPRVRRRR
RTRRRTYRRGWRRRRYIRRGRRKKKLILTQWNPAIVKRCNIKGGLPIIIC
GEPRAAFNYGYHMEDYTPQPFPFGGGMSTVTFSLKALYDQYLKHQNRWTF
SNDQLDLARYRGCKLRFYRSPVCDFIVHYNLIPPLKMNQFTSPNTHPGLL
MLSKHKIIIPSFQTRPGGRRFVKIRLNPPKLFEDKWYTQQDLCKVPLVSI
TATAADLRYPFCSPQTNNPCTTFQVLRKNYNTVIGTSVKDQESTQDFENW
LYKTDSHYQTFATEAQLGRIPAFNPDGTKNTKQQSWQDNWSKKNSPWTGN
SGTYPQTTSEMYKIPYDSNFGFPTYRAQKDYILERRQCNFNYEVNNPVSK
KVWPQPSTTTPTVDYYEYHCGWFSNIFIGPNRYNLQFQTAYVDTTYNPLM
DKGKGNKIWFQYLSKKGTDYNEKQCYCTLEDMPLWAICFGYTDYVETQLG
PNVDHETAGLIIMICPYTQPPMYDKNRPNWGYVVYDTNFGNGKMPSGSGQ
VPVYWQCRWRPMLWFQQQVLNDISKTGPYAYRDEYKNVQLTLYYNFIENW
GGDMYYPQVVKNPCGDSGIVPGSGRFTREVQVVSPLSMGPAYIFHYFDSR
RGFFSEKALKRMQQQQEFDESFTFKPKRPKLSTAAAEILQLEEDSTSGEG
KSPLQQEEKEVEVLQTPTVQLQLQRNIQEQLAIKQQLQFLLLQLLKTQSN
LHLNPQFLSPS (SEQ ID NO: 197)
Annotations:

| Putative Domain | AA range |
|---|---|
| Arg-Rich Region | 1-75 |
| Jelly-roll domain | 75-284 |
| Hypervariable Region | 285-432 |
| N22 | 433-599 |
| C-terminal Domain | 600-780 |

TABLE 29
Exemplary Anellovirus ORF1 amino acid
subsequence (Alphatorquevirus, Clade 5)
TTV-16(TUS01)-ORF1 (Alphatorquevirus Clade 5)

| | |
|---|---|
| Arg-Rich | MAYWFRRWGWRPRRRWRRWRRRRRRLPRRRTRRAVRGLG |
| Region | RRRKPRVRRRRRRRRTYRRGWRRRRYIRRGRRKKK |
| | (SEQ ID NO: 198) |
| Jelly- | LILTQWNPAIVKRCNIKGGLPIIICGEPRAAFNYGYHME |
| roll | DYTPQPFPFGGGMSTVTFSLKALYDQYLKHQNRWTFSND |
| Domain | QLDLARYRGCKLRFYRSPVCDFIVHYNLIPPLKMNQFTS |
| | PNTHPGLLMLSKHKIIIPSFQTRPGGRRFVKIRLNPPKL |
| | FEDKWYTQQDLCKVPLVSITATAADLRYPFCSPQTNNPC |
| | TTFQVLRKNYNTVI (SEQ ID NO: 199) |
| Hyper- | GTSVKDQESTQDFENWLYKTDSHYQTFATEAQLGRIPAF |
| variable | NPDGTKNTKQQSWQDNWSKKNSPWTGNSGTYPQTTSEMY |
| domain | KIPYDSNFGFPTYRAQKDYILERRQCNFNYEVNNPVSKK |
| | VWPQPSTTTPTVDYYEYHCGWFSNIFIGPNR (SEQ ID |
| | NO: 200) |
| N22 | YNLQFQTAYVDTTYNPLMDKGKGNKIWFQYLSKKGTDYN |
| | EKQCYCTLEDMPLWAICFGYTDYVETQLGPNVDHETAGL |
| | IIMICPYTQPPMYDKNRPNWGYVVYDTNFGNGKMPSGSG |
| | QVPVYWQCRWRPMLWFQQQVLNDISKTGPYAYRDEYKNV |
| | QLTLYYNFIFN (SEQ ID NO: 201) |
| C- | WGGDMYYPQVVKNPCGDSGIVPGSGRFTREVQVVSPLSM |
| terminal | GPAYIFHYFDSRRGFFSEKALKRMQQQQEFDESFTFKPK |
| domain | RPKLSTAAAEILQLEEDSTSGEGKSPLQQEEKEVEVLQT |
| | PTVQLQLQRNIQEQLAIKQQLQFLLLQLLKTQSNLHLNP |
| | QFLSPS (SEQ ID NO: 202) |

TABLE 30
Exemplary Anellovirus ORF1 amino acid
subsequence (Alphatorquevirus, Clade 6)

| | |
|---|---|
| Name | TTV-TJN02 |
| Genus/Clade | Alphatorquevirus, Clade 6 |
| Accession Number | AB028669.1 |
| Protein Accession Number | BAA94878.1 |

Full Sequence: 746 AA
1 10 20 30 40 50
| | | | | |
MAWGWWRWRRRWPARRWRRRRRRRPVRRTRARRPARRYRRRRTVRTRRRR
WGRRRYRRGWRRRTYVRKGRHRKKKKRLILRQWQPATRRRCTITGYLPIV
FCGHTRGNKNYALHSDDYTPQGQPFGGALSTTSFSLKVLFDQHORGLNKW
SFPNDQLDLARYRGCKFIFYRTKQTDWVGQYDISEPYKLDKYSCPNYHPG
NMIKAKHKFLIPSYDTNPRGRQKIIVKIPPPDLFVDKWYTQEDLCSVNLV
SLAVSAASFLHPFGSPQTDNPCYTFQVLKEFYYQAIGFSASTQAMTSVLD
TLYTQNSYWESNLTQFYVLNAKKGSDTTQPLTSNMPTREEFMAKKNTNYN
WYTYKAASVKNKLHQMRQTYFEELTSKGPQTTKSEEGYSQHWTTPSTNAY
EYHLGMFSAIFLAPDRPVPRFPCAYQDVTYNPLMDKGVGNHIWFQYNTKA
DTQLIVTGGSCKAHIQDIPLWAAFYGYSDFIESELGPFVDAETVGLVCVI
CPYTKPPMYNKTNPAMGYVFYDRNFGDGKWTDGRGKIEPYWQVRWRPEML
FQETVMADLVQTGPFSYKDELKNSTLVCKYKFYFTWGGNMMFQQTIKNPC
KTDGQPTDSSRHPRGIQVADPEQMGPRWVFHSFDWRRGYLSEKALKRLQE
KPLDYDEYFTQPKRPRIFPPTESAEGEFREPEKGSYSEEERSQASAEEQT
QEATVLLLKRRLREQQQLQQQLQFLTREMFKTQAGLHLNPMLLNQR
(SEQ ID NO: 203)
Annotations:

| Putative Domain | AA range |
|---|---|
| Arg-Rich Region | 1-77 |
| Jelly-roll domain | 78-286 |
| Hypervariable Region | 287-416 |
| N22 | 417-585 |
| C-terminal Domain | 586-746 |

TABLE 31
Exemplary Anellovirus ORF1 amino acid
subsequence (Alphatorquevirus, Clade 6)
TTV-TJN02-ORF1 (Alphatorquevirus Clade 6)

| | |
|---|---|
| Arg-Rich | MAWGWWRWRRRWPARRWRRRRRRRPVRRTRARRPARRYR |
| Region | RRRTVRTRRRRWGRRRYRRGWRRRTYVRKGRHRKKKKR |
| | (SEQ ID NO: 204) |
| Jelly- | LILRQWQPATRRRCTITGYLPIVFCGHTRGNKNYALHSD |
| roll | DYTPQGQPFGGALSTTSFSLKVLFDQHQRGLNKWSFPND |
| Domain | QLDLARYRGCKFIFYRTKQTDWVGQYDISEPYKLDKYSC |
| | PNYHPGNMIKAKHKFLIPSYDTNPRGRQKIIVKIPPPDL |
| | FVDKWYTQEDLCSVNLVSLAVSAASFLHPFGSPQTDNPC |
| | YTFQVLKEFYYQAI (SEQ ID NO: 205) |
| Hyper- | GFSASTQAMTSVLDTLYTQNSYWESNLTQFYVLNAKKGS |
| variable | DTTQPLTSNMPTREEFMAKKNTNYNWYTYKAASVKNKLH |
| domain | QMRQTYFEELTSKGPQTTKSEEGYSQHWTTPSTNAYEYH |
| | LGMFSAIFLAPDR (SEQ ID NO: 206) |
| N22 | PVPRFPCAYQDVTYNPLMDKGVGNHIWFQYNTKADTQLI |
| | VTGGSCKAHIQDIPLWAAFYGYSDFIESELGPFVDAETV |
| | GLVCVICPYTKPPMYNKTNPAMGYVFYDRNFGDGKWTDG |
| | RGKIEPYWQVRWRPEMLFQETVMADLVQTGPFSYKDELK |
| | NSTLVCKYKFYFT (SEQ ID NO: 207) |
| C- | WGGNMMFQQTIKNPCKTDGQPTDSSRHPRGIQVADPEQM |
| terminal | GPRWVFHSFDWRRGYLSEKALKRLQEKPLDYDEYFTQPK |
| domain | RPRIFPPTESAEGEFREPEKGSYSEEERSQASAEEQTQE |
| | ATVLLLKRRLREQQQLQQQLQFLTREMFKTQAGLHLNPM |
| | LLNQR (SEQ ID NO: 208) |

TABLE 32
Exemplary Anellovirus ORF1 amino acid
subsequence (Alphatorquevirus, Clade 7)

| | |
|---|---|
| Name | TTV-HD16d |
| Genus/Clade | Alphatorquevirus, Clade 7 |
| Accession Number | FR751479.1 |
| Protein Accession Number | NA |

Full Sequence: 765 AA
1 10 20 30 40 50
| | | | | |
MAWSWWWQRWRRRRWKPRRRRWRRLRWRRPRRAVRRRRRGRRVRRRRWAR
RRGRRRRYATRRKRRYRGRRFKKKLVLTQWHPNTMRRCLIKGIVPLVICG
HTRWNYNYALHSKDYTEEGRYPHGGALSTTTWSLKVLYDEHLKHHDFWGY
PNNQLDLARYKGAKFTFYRHKKTDFIIFFNRKPPFKLNKYSCASYHPGML
MQQRHKILLPSYETKPKGRPKITVRIKPPTLLEDKWYTQQDLCDVNLLQL
VVTAADFRHPLCSPQTNTPTTTFQVLKDIYYDTMSISEPTDSYTSVNNKS
TTQTFTNYSNTLENILYTRASYWNSFHATEYLNPNIIYKNGEKLFKEHED
LITWMTQTNNTGFLTKNNTAFGNNSYRPNADKIKKARKTYWNALIGTNDL
ATNIGQARAERFEYHLGWYSPIFLSRHRSNMNFARAYQDVTYNPNCDRGV
NNRVWVQPLTKPTTEFDEKRCKCVVQHLPLWAALYCYQDFVEEELGSSSE
ILNSCLLVLQCPYTFPPMYDKKLPDKGFVFYDSLFGDGKMSDGRGQVDIF
WQQRWYPRLATQMQVMHDITMTGPFSYRDELVSTQLTAKYTFDEMWGGNM
ISTQIIKNPCKDSGLEPAYPGRQRRDLQIVDPYSMGPQFSFHNWDYRHGL
FGQDAIDRVSKQPKDDADYPNPYKRPRYFPPTDQAAQEQEKDFSFLKTAP
SNSEESDQEVLQETQVLRFQPEQHKQLHLQLAERQRIGEQLRYLLQQMFK
TQANLHLNPYTFTQL (SEQ ID NO: 209)
Annotations:

| Putative Domain | AA range |
|---|---|
| Arg-Rich Region | 1-74 |
| Jelly-roll domain | 75-286 |
| Hypervariable Region | 287-428 |
| N22 | 429-595 |
| C-terminal Domain | 596-765 |

TABLE 33
Exemplary Anellovirus ORF1 amino acid
subsequence (Alphatorquevirus, Clade 7)
TTV-HD16d-ORF1 (Alphatorquevirus Clade 7)

| | |
|---|---|
| Arg-Rich | MAWSWWWQRWRRRRWKPRRRRWRRLRWRRPRRAVRRRRR |
| Region | GRRVRRRRWARRRGRRRRYATRRKRRYRGRRFKKK |
| | (SEQ ID NO: 210) |
| Jelly- | LVLTQWHPNTMRRCLIKGIVPLVICGHTRWNYNYALHSK |
| roll | DYTEEGRYPHGGALSTTTWSLKVLYDEHLKHHDFWGYPN |
| Domain | NQLDLARYKGAKFTFYRHKKTDFIIFFNRKPPFKLNKYS |
| | CASYHPGMLMQQRHKILLPSYETKPKGRPKITVRIKPPT |
| | LLEDKWYTQQDLCDVNLLQLVVTAADFRHPLCSPQTNTP |
| | TTTFQVLKDIYYDTMSI (SEQ ID NO: 211) |
| Hyper- | SEPTDSYTSVNNKSTTQTFTNYSNTLENILYTRASYWNS |
| variable | FHATEYLNPNIIYKNGEKLFKEHEDLITWMTQTNNTGFL |
| domain | TKNNTAFGNNSYRPNADKIKKARKTYWNALIGTNDLATN |
| | IGQARAERFEYHLGWYSPIFLSRHR (SEQ ID NO: |
| | 212) |
| N22 | SNMNFARAYQDVTYNPNCDRGVNNRVWVQPLTKPTTEFD |
| | EKRCKCVVQHLPLWAALYCYQDFVEEELGSSSEILNSCL |
| | LVLQCPYTFPPMYDKKLPDKGFVFYDSLFGDGKMSDGRG |
| | QVDIFWQQRWYPRLATQMQVMHDITMTGPFSYRDELVST |
| | QLTAKYTFDFM (SEQ ID NO: 213) |
| C- | WGGNMISTQIIKNPCKDSGLEPAYPGRQRRDLQIVDPYS |
| terminal | MGPQFSFHNWDYRHGLFGQDAIDRVSKQPKDDADYPNPY |
| domain | KRPRYFPPTDQAAQEQEKDFSFLKTAPSNSEESDQEVLQ |
| | ETQVLRFQPEQHKQLHLQLAERQRIGEQLRYLLQQMFKT |
| | QANLHLNPYTFTQL (SEQ ID NO: 214) |

TABLE 34
Exemplary Anellovirus ORF1 amino acid
subsequence (Betatorquevirus)

| | |
|---|---|
| Name | Ring2 |
| Genus/Clade | Betatorquevirus |
| Accession Number | JX134045.1 |
| Protein Accession Number | AGG91484.1 |

Full Sequence: 666 AA
1 10 20 30 40 50
| | | | | |
MPYYYRRRRYNYRRPRWYGRGWIRRPFRRRFRRKRRVRPTYTTIPLKQWQ
PPYKRTCYIKGQDCLIYYSNLRLGMNSTMYEKSIVPVHWPGGGSFSVSML
TLDALYDIHKLCRNWWTSTNQDLPLVRYKGCKITFYQSTFTDYIVRIHTE
LPANSNKLTYPNTHPLMMMMSKYKHIIPSRQTRRKKKPYTKIFVKPPPQF
ENKWYFATDLYKIPLLQIHCTACNLQNPFVKPDKLSNNVTLWSLNTISIQ
NRNMSVDQGQSWPFKILGTQSFYFYFYTGANLPGDTTQIPVADLLPLINP
RINRPGQSLNEAKITDHITFTEYKNKFTNYWGNPENKHIQEHLDMILYSL
KSPEAIKNEWTTENMKWNQLNNAGTMALTPFNEPIFTQIQYNPDRDTGED
TQLYLLSNATGTGWDPPGIPELILEGFPLWLIYWGFADFQKNLKKVTNID
TNYMLVAKTKFTQKPGTFYLVILNDTFVEGNSPYEKQPLPEDNIKWYPQV
QYQLEAQNKLLQTGPFTPNIQGQLSDNISMFYKFYFKWGGSPPKAINVEN
PAHQIQYPIPRNEHETTSLQSPGEAPESILYSFDYRHGNYTTTALSRISQ
DWALKDTVSKITEPDRQQLLKQALECLQISEETQEKKEKEVQQLISNLRQ
QQQLYRERIISLLKDQ (SEQ ID NO: 215)
Annotations:

| Putative Domain | AA range |
|---|---|
| Arg-Rich Region | 1-38 |
| Jelly-roll domain | 39-246 |
| Hypervariable Region | 247-374 |
| N22 | 375-537 |
| C-terminal Domain | 538-666 |

TABLE 35
Exemplary Anellovirus ORF1 amino acid
subsequence (Betatorquevirus)
Ring2 ORF1 (Betatorquevirus)

| | |
|---|---|
| Arg-Rich | MPYYYRRRRYNYRRPRWYGRGWIRRPFRRRFRRKRRVR |
| Region | (SEQ ID NO: 216) |
| Jelly- | PTYTTIPLKQWQPPYKRTCYIKGQDCLIYYSNLRLGMNS |
| roll | TMYEKSIVPVHWPGGGSFSVSMLTLDALYDIHKLCRNWW |
| Domain | TSTNQDLPLVRYKGCKITFYQSTFTDYIVRIHTELPANS |
| | NKLTYPNTHPLMMMMSKYKHIIPSRQTRRKKKPYTKIFV |
| | KPPPQFENKWYFATDLYKIPLLQIHCTACNLQNPFVKPD |
| | KLSNNVTLWSLNT (SEQ ID NO: 217) |
| Hyper- | ISIQNRNMSVDQGQSWPFKILGTQSFYFYFYTGANLPGD |
| variable | TTQIPVADLLPLTNPRINRPGQSLNEAKITDHITFTEYK |
| domain | NKFTNYWGNPFNKHIQEHLDMILYSLKSPEAIKNEWTTE |
| | NMKWNQLNNAG (SEQ ID NO: 218) |
| N22 | TMALTPFNEPIFTQIQYNPDRDTGEDTQLYLLSNATGTG |
| | WDPPGIPELILEGFPLWLIYWGFADFQKNLKKVTNIDTN |
| | YMLVAKTKFTQKPGTFYLVILNDTFVEGNSPYEKQPLPE |
| | DNIKWYPQVQYQLEAQNKLLQTGPFTPNIQGQLSDNISM |
| | FYKFYFK (SEQ ID NO: 219) |
| C- | WGGSPPKAINVENPAHQIQYPIPRNEHETTSLOSPGEAP |
| terminal | ESILYSFDYRHGNYTTTALSRISQDWALKDTVSKITEPD |
| domain | RQQLLKQALECLQISEETQEKKEKEVQQLISNLRQQQQL |
| | YRERIISLLKDQ (SEQ ID NO: 220) |

TABLE 36
Exemplary Anellovirus ORF1 amino acid
subsequence (Gammatorquevirus)

| | |
|---|---|
| Name | TTMDV-MD1-073 |
| Genus/Clade | Gammatorquevirus |
| Accession Number | AB290918.1 |
| Protein Accession Number | BAG49427.1 |

Full Sequence: 673 AA
1 10 20 30 40 50
| | | | | |
MPFWWGRRNKFWYGRNYRRKKRRFPKRRKRRFYRRTKYRRPARRRRRRRR
KVRRKKKTLIVRQWQPDSIVLCKIKGYDSIIWGAEGTQFQCSTHEMYEYT
RQKYPGGGGFGVQLYSLEYLYDQWKLRNNIWTKTNQLKDLCRYLKCVMTF
YRHQHIDFVIVYERQPPFEIDKLTYMKYHPYMLLQRKHKIILPSQTTNPR
GKLKKKKTIKPPKQMLSKWFFQQQFAKYDLLLIAAAACSLRYPRIGCCNE
NRMITLYCLNTKFYQDTEWGTTKQAPHYFKPYATINKSMIFVSNYGGKKT
EYNIGQWIETDIPGEGNLARYYRSISKEGGYFSPKILQAYQTKVKSVDYK
PLPIVLGRYNPAIDDGKGNKIYLQTIMNGHWGLPQKTPDYIIEEVPLWLG
FWGYYNYLKQTRTEAIFPLHMFVVQSKYIQTQQTETPNNFWAFIDNSFIQ
GKNPWDSVITYSEQKLWFPTVAWQLKTINAICESGPYVPKLDNQTYSTWE
LATHYSFHFKWGGPQISDQPVEDPGNKNKYDVPDTIKEALQIVNPAKNIA
ATMFHDWDYRRGCITSTAIKRMQQNLPTDSSLESDSDSEPAPKKKRLLPV
LHDPQKKTEKINQCLLSLCEESTCQEQETEENILKLIQQQQQQQQKLKHN
LLVLIKDLKVKQRLLQLQTGVLE (SEQ ID NO: 221)
Annotations:

| Putative Domain | AA range |
|---|---|
| Arg-Rich Region | 1-57 |
| Jelly-roll domain | 58-259 |
| Hypervariable Region | 260-351 |
| N22 | 352-510 |
| C-terminal Domain | 511-673 |

TABLE 37
Exemplary Anellovirus ORF1 amino acid
subsequence (Gammatorquevirus)
TTV-HD16d-ORF1 (Gammatorquevirus)

| | |
|---|---|
| Arg-Rich | MPFWWGRRNKFWYGRNYRRKKRRFPKRRKRRFYRRTKYR |
| Region | RPARRRRRRRRKVRRKKK (SEQ ID NO: 222) |
| Jelly- | TLIVRQWQPDSIVLCKIKGYDSIIWGAEGTQFQCSTHEM |
| roll | YEYTRQKYPGGGGFGVQLYSLEYLYDQWKLRNNIWTKTN |
| Domain | QLKDLCRYLKCVMTFYRHQHIDFVIVYERQPPFEIDKLT |
| | YMKYHPYMLLQRKHKIILPSQTTNPRGKLKKKKTIKPPK |
| | QMLSKWFFQQQFAKYDLLLIAAAACSLRYPRIGCCNENR |
| | MITLYCL (SEQ ID NO: 223) |
| Hyper- | NTKFYQDTEWGTTKQAPHYFKPYATINKSMIFVSNYGGK |
| variable | KTEYNIGQWIETDIPGEGNLARYYRSISKEGGYFSPKIL |
| domain | QAYQTKVKSVDYKP (SEQ ID NO: 224) |
| N22 | LPIVLGRYNPAIDDGKGNKIYLQTIMNGHWGLPQKTPDY |
| | IIEEVPLWLGFWGYYNYLKQTRTEAIFPLHMFVVQSKYI |
| | QTQQTETPNNFWAFIDNSFIQGKNPWDSVITYSEQKLWF |
| | PTVAWQLKTINAICESGPYVPKLDNQTYSTWELATHYSF |
| | HFK (SEQ ID NO: 225) |
| C- | WGGPQISDQPVEDPGNKNKYDVPDTIKEALQIVNPAKNI |
| terminal | AATMFHDWDYRRGCITSTAIKRMQQNLPTDSSLESDSDS |
| domain | EPAPKKKRLLPVLHDPQKKTEKINQCLLSLCEESTCQEQ |
| | ETEENILKLIQQQQQQQQKLKHNLLVLIKDLKVKQRLLQ |
| | LQTGVLE (SEQ ID NO: 226) |

TABLE D1
Exemplary Anellovirus ORF1 amino acid
subsequence (Gammatorquevirus)

| | |
|---|---|
| Name | Ring 3.1 |
| Genus/Clade | Gammatorquevirus |
| Accession Number | |
| Protein Accession Number | |

Full Sequence: 677 AA
1 10 20 30 40 50
| | | | | |
MPFWWRRRNKRWWGRRFRYRRYNKYKTRRRRRIPRRRNRRFTKTRRRRKR
KKVRRKLKKITIKQWQPDSVKKCKIKGYSTLVMGAQGKQYNCYTNQASDY
VQPKAPQGGGFGCEVFNLKWLYQEYTAHRNIWTKTNEYTDLCRYTGAQII
LYRHPDVDFIVSWDNQPPFLLNKYTYPELQPQNLLLARRKRIILSQKSNP
KGKLRIKLRIPPPKQMITKWFFQRDFCDVNLFKLCASAASFRYPGISHGA
QSTIFSAYALNTDFYQCSDWCQTNTETGYLNIKTQQMPLWFHYREGGKEK
WYKYTNKEHRPYTNTYLKSISYNDGLFSPKAMFAFEVKAGGEGTTEPPQG
AQLIANLPLIALRYNPHEDTGHGNEIYLTSTFKGTYDKPKVTDALYENNV
PLWMGFYGYWDFILQETKNKGVFDQHMFVVKCPALRPISQVTKQVYYPLV
DMDFCSGRLPFDEYLSKDIKSHWYPTAERQTVTINNFVTAGPYMPKFEPT
DKDSTWQLNYHYKFFFKWGGPQVTDPTVEDPCSRNKYPVPDTMQQTIQIK
NPEKLHPATLFHDWDLRRGFITQAAIKRMSENLQIDSSFESDGTESPKKK
KRCTKEIPTQNQKQEEIQECLLSLCEEPTCQEETEDLQLFIQQQQQQQYK
LRKNLFKLLTHLKKGQRISQLQTGLLE (SEQ ID NO: 919)
Annotations:

| Putative Domain | AA range |
|---|---|
| Arg-Rich Region | 1-59 |
| Jelly-roll domain | 60-260 |
| Hypervariable Region | 261-356 |
| N22 | 357-517 |
| C-terminal Domain | 518-677 |

TABLE D2
Exemplary Anellovirus ORF1 amino acid subsequence (Gammatorquevirus)
Ring3.1 (Gammatorquevirus)

| | |
|---|---|
| Arg-Rich | MPFWWRRRNKRWWGRRFRYRRYNKYKTRRRRRIPRRRNRRFTKTRR |
| Region | RRKRKKVRRKLKK (SEQ ID NO: 920) |
| Jelly-roll | ITIKQWQPDSVKKCKIKGYSTLVMGAQGKQYNCYTNQASDYVQPKAP |
| Domain | QGGGFGCEVFNLKWLYQEYTAHRNIWTKTNEYTDLCRYTGAQIILYR |
| | HPDVDFIVSWDNQPPFLLNKYTYPELQPQNLLLARRKRIILSQKSNPKG |
| | KLRIKLRIPPPKQMITKWFFQRDFCDVNLFKLCASAASFRYPGISHGAQS |
| | TIFSAYAL (SEQ ID NO: 921) |
| Hypervariable | NTDFYQCSDWCQTNTETGYLNIKTQQMPLWFHYREGGKEKWYKYTN |
| domain | KEHRPYTNTYLKSISYNDGLFSPKAMFAFEVKAGGEGTTEPPQGAQLIA |
| | N (SEQ ID NO: 922) |
| N22 | LPLIALRYNPHEDTGHGNEIYLTSTFKGTYDKPKVTDALYFNNVPLWM |
| | GFYGYWDFILQETKNKGVFDQHMFVVKCPALRPISQVTKQVYYPLVD |
| | MDFCSGRLPFDEYLSKDIKSHWYPTAERQTVTINNFVTAGPYMPKFEPT |
| | DKDSTWQLNYHYKFFFK (SEQ ID NO: 923) |
| C-terminal | WGGPQVTDPTVEDPCSRNKYPVPDTMQQTIQIKNPEKLHPATLFHDWD |
| domain | LRRGFITQAAIKRMSENLQIDSSFESDGTESPKKKKRCTKEIPTQNQKQE |
| | EIQECLLSLCEEPTCQEETEDLQLFIQQQQQQQYKLRKNLFKLLTHLKK |
| | GQRISQLQTGLLE (SEQ ID NO: 924) |

TABLE D3
Exemplary Anellovirus ORF1 amino acid subsequence (Gammatorquevirus)

| | |
|---|---|
| Name | Ring 4 |
| Genus/Clade | Gammatorquevirus |
| Accession Number | |
| Protein Accession Number | |

Full Sequence: 662 AA
1 10 20 30 40 50
| | | | | |
MPFWWRRRRKFWTNNRFNYTKRRRYRKRWPRRRRRRRPYRRPVRRRRRKL
RKVKRKKKSLIVRQWQPDSIRTCKIIGQSAIVVGAEGKQMYCYTVNKLIN
VPPKTPYGGGFGVDQYTLKYLYEEYRFAQNIWTQSNVLKDLCRYINVKLI
FYRDNKTDFVLSYDRNPPFQLTKFTYPGAHPQQIMLQKHHKFILSQMTKP
NGRLTKKLKIKPPKQMLSKWFFSKQFCKYPLLSLKASALDLRHSYLGCCN
ENPQVFFYYLNHGYYTITNWGAQSSTAYRPNSKVIDTTYYRYKNDRKNIN
IKSHEYEKSISYENGYFQSSFLQTQCIYTSERGEACIAEKPLGIAIYNPV
KDNGDGNMIYLVSTLANTWDQPPKDSAILIQGVPIWLGLFGYLDYCRQIK
ADKTWLDSHVLVIQSPAIFTYPNPGAGKWYCPLSQSFINGNGPFNQPPTL
LQKAKWFPQIQYQQEIINSFVESGPFVPKYANQTESNWELKYKYVFTFKW
GGPQFHEPEIADPSKQEQYDVPDTFYQTIQIEDPEGQDPRSLIHDWDYRR
GFIKERSLKRMSTYFSTHTDQQATSEEDIPKKKKRIGPQLTVPQQKEEET
LSCLLSLCKKDTFQETETQEDLQQLIKQQQEQQLLLKRNILQLIHKLKEN
QQMLQLHTGMLP(SEQ ID NO: 925)
Annotations:

| Putative Domain | AA range |
|---|---|
| Arg-Rich Region | 1-58 |
| Jelly-roll domain | 59-260 |
| Hypervariable Region | 261-339 |
| N22 | 340-499 |
| C-terminal Domain | 500-662 |

TABLE D4
Exemplary Anellovirus ORF1 amino acid subsequence (Gammatorquevirus)
Ring4 (Gammatorquevirus)

| | |
|---|---|
| Arg-Rich | MPFWWRRRRKFWTNNRFNYTKRRRYRKRWPRRRRRRRPYRRPVRRR |
| Region | RRKLRKVKRKKK (SEQ ID NO: 926) |
| Jelly-roll | SLIVRQWQPDSIRTCKIIGQSAIVVGAEGKQMYCYTVNKLINVPPKTPY |
| Domain | GGGFGVDQYTLKYLYEEYRFAQNIWTQSNVLKDLCRYINVKLIFYRDN |
| | KTDFVLSYDRNPPFQLTKFTYPGAHPQQIMLQKHHKFILSQMTKPNGR |
| | LTKKLKIKPPKQMLSKWFFSKQFCKYPLLSLKASALDLRHSYLGCCNE |
| | NPQVFFYYL (SEQ ID NO: 927) |
| Hypervariable | NHGYYTITNWGAQSSTAYRPNSKVTDTTYYRYKNDRKNINIKSHEYEK |
| domain | SISYENGYFQSSFLQTQCIYTSERGEACIAE (SEQ ID NO: 928) |
| N22 | KPLGIAIYNPVKDNGDGNMIYLVSTLANTWDQPPKDSAILIQGVPIWLG |
| | LFGYLDYCRQIKADKTWLDSHVLVIQSPAIFTYPNPGAGKWYCPLSQSF |
| | INGNGPFNQPPTLLQKAKWFPQIQYQQEIINSFVESGPFVPKYANQTESN |
| | WELKYKYVFTFK (SEQ ID NO: 929) |
| C-terminal | WGGPQFHEPEIADPSKQEQYDVPDTFYQTIQIEDPEGQDPRSLIHDWDY |
| domain | RRGFIKERSLKRMSTYFSTHTDQQATSEEDIPKKKKRIGPQLTVPQQKE |
| | EETLSCLLSLCKKDTFQETETQEDLQQLIKQQQEQQLLLKRNILQLIHKL |
| | KENQQMLQLHTGMLP (SEQ ID NO: 930) |

TABLE D5
Exemplary Anellovirus ORF1 amino acid subsequence (Alphatorquevirus)
Clade 1

| | |
|---|---|
| Name | Ring 5.2 |
| Genus/Clade | Alphatorquevirus Clade 1 |
| Accession Number | |

Protein Accession Number
Full Sequence: 728 AA
1 10 20 30 40 50
| | | | | |
TAWWWGRWRRRWRRRRPYTTRLRRRRARRAFPRRRRRRFVSRRWRRPYRR
RRRRGRRRRRRRRRHKPTLILRQWQPDCIRHCKITGWMPLIICGKGSTQF
NYITHADDITPRGASYGGNFTNMTFSLEAIYEQFLYHRNRWSASNHDLEL
CRYKGTTLKLYRHPEVDYIVTYSRTGPFEISHMTYLSTHPMLMLLNKHHI
VVPSLKTKPRGRKAIKVRIRPPKLMNNKWYFTRDFCNIGLFQLWATGLEL
RNPWLRMSTLSPCIGENVLKNSIYTNLSNLPQYKNERLNIINNILHPQEI
TGTNNKKWQYTYTKLMAPIYYSANRASTYDWENYSKETNYNNTYVKFTQK
RQEKLTKIRKEWQMLYPQQPTALPDSYDLLQEYGLYSPYYLNPTRINLDW
MTPYTHVRYNPLVDKGFGNRIYIQWCSEADVSYNRTKSKCLLQDMPLFFM
CYGYIDWAIKNTGVSSLVKDARICIRCPYTEPQLVGSTEDIGFVPISETF
MRGDMPVLAPYIPLSWFCKWYPNIAHQKEVLESIISCSPFMPRDQDMNGW
DITIGYKMDFLWGGSPLPSQPIDDPCQQGTHPIPDPDKHPRLLQVSNPKL
LGPRTVFHKWDIRRGQFSKRSIKRVSEYSSDDESLAPGLPSKRNKLDSAF
RGENREQKECYSLLKALEEEETPEEEEPAPQEKAQKEELLHQLQLQRRHQ
RVLRRGLKLVFTDILRLRQGVHWNPELT (SEQ ID NO: 931)
Annotations:

| Putative Domain | AA range |
|---|---|
| Arg-Rich Region | 1-66 |
| Jelly-roll domain | 67-277 |
| Hypervariable Region | 278-395 |
| N22 | 396-561 |
| C-terminal Domain | 562-728 |

TABLE D6
Exemplary Anellovirus ORF1 amino acid subsequence (Alphatorquevirus) Clade 1
Ring5.2 (Alphatorquevirus) Clade 1

| | |
|---|---|
| Arg-Rich | TAWWWGRWRRRWRRRRPYTTRLRRRRARRAFPRRRRRRFVSRRWRR |
| Region | PYRRRRRRGRRRRRRRRRHK (SEQ ID NO: 932) |
| Jelly-roll | PTLILRQWQPDCIRHCKITGWMPLIICGKGSTQFNYITHADDITPRGASY |
| Domain | GGNFTNMTFSLEAIYEQFLYHRNRWSASNHDLELCRYKGTTLKLYRHP |
| | EVDYIVTYSRTGPFEISHMTYLSTHPMLMLLNKHHIVVPSLKTKPRGRK |
| | AIKVRIRPPKLMNNKWYFTRDFCNIGLFQLWATGLELRNPWLRMSTLS |
| | PCIGFNVLKNSIYTNL (SEQ ID NO: 933) |
| Hypervariable | SNLPQYKNERLNIINNILHPQEITGTNNKKWQYTYTKLMAPIYYSANRA |
| domain | STYDWENYSKETNYNNTYVKFTQKRQEKLTKIRKEWQMLYPQQPTAL |
| | PDSYDLLQEYGLYSPYYLNPTR (SEQ ID NO: 934) |
| N22 | INLDWMTPYTHVRYNPLVDKGFGNRIYIQWCSEADVSYNRTKSKCLL |
| | QDMPLFFMCYGYIDWAIKNTGVSSLVKDARICIRCPYTEPQLVGSTEDI |
| | GFVPISETFMRGDMPVLAPYIPLSWFCKWYPNIAHQKEVLESIISCSPFM |
| | PRDQDMNGWDITIGYKMDFL (SEQ ID NO: 935) |
| C-terminal | WGGSPLPSQPIDDPCQQGTHPIPDPDKHPRLLQVSNPKLLGPRTVFHKW |
| domain | DIRRGQFSKRSIKRVSEYSSDDESLAPGLPSKRNKLDSAFRGENREQKE |
| | CYSLLKALEEEETPEEEEPAPQEKAQKEELLHQLQLQRRHQRVLRRGL |
| | KLVFTDILRLRQGVHWNPELT (SEQ ID NO: 936) |

TABLE D7
Exemplary Anellovirus ORF1 amino acid subsequence (Alphatorquevirus)-
Clade 3

| | |
|---|---|
| Name | Ring6.0 |
| Genus/Clade | Alphatorquevirus Clade 3 |
| Accession Number | |

Protein Accession Number
Full Sequence: 767 AA
1 10 20 30 40 50
| | | | | |
MAYGWWRRRRRRPWWRRRWRRWRRRRRPRRRRPRRRYRRRRTVRRRGRGR
WTRAHRRWRRKGKRSRKKKIIIRQWQPNYTRRCNIVGYMPLLICGENTVA
TNYATHSDDSYYPGPFGGGMTTDKFTLRILYDEYKREMNYWTSSNEDLDL
CRYLGCTLYVFRHPEVDFIIIINTSPPFLDTEITGPSIHPGMMALNKRSR
WIPSIKNRPGRKHYIKIKVGAPRMFTDKWYPQTDLCDMTLLTIFASAADM
QYPFGSPLTDTIVVSFQVLQSMYNDCLSVLPDNFAETSGKGTQLHENIIQ
HLPYYNTTQTQAQFKRFIENMNATNGDNIWASYINTTKFSSANTPKNDTG
IGGPYTTYSDSWYKGTVYNDKIKTIPIKASKLYYEQTKNLIGITFTGSTH
RLHYCGGLYSSVWLSAGRSYFETKGPYTDITYNPFSDRGEGNMLWIDWLT
KNDSVYSKTSSKCLIENLPLWASVYGYKEYCSKVTGDTNIEHNCRCVIRS
PYTVPQLLDHNNPFRGYVPYSFNFGNGKMPGGSSLVPIRMRAKWYPTLFH
QKEVLEAIAQAGPFAYHSDIKKVSLGIKYRFKWVWGGNPVSQQVVRNPCK
TTQGSSGNRVPRSIQVVDPRYNTPELTIHAWDFRHGFFGRKAIKRMQEQP
IPHDTFSAGFKRSRRDTEALQCSQEEQQKENLLFPVQQLKRVPPWETSQE
SQSEEENSQKQETLSQQLRDQLHKQRLMGEQLRSLLYQMQRVQQNQHINP
MLLPKGLALTSISHNVI (SEQ ID NO: 937)
Annotations:

| Putative Domain | AA range |
|---|---|
| Arg-Rich Region | 1-69 |
| Jelly-roll domain | 70-269 |
| Hypervariable Region | 270-424 |
| N22 | 425-584 |
| C-terminal Domain | 585-767 |

TABLE D8
Exemplary Anellovirus ORF1 amino acid subsequence (Alphatorquevirus)-Clade 3
Ring6.0 (Alphatorquevirus)

| Arg-Rich | MAYGWWRRRRRRPWWRRRWRRWRRRRRPRRRRPRRRYRRRRTVRR |
|---|---|
| Region | RGRGRWTRAHRRWRRKGKRSRKKK (SEQ ID NO: 938) |
| Jelly-roll | IIIRQWQPNYTRRCNIVGYMPLLICGENTVATNYATHSDDSYYPGPFGG |
| Domain | GMTTDKFTLRILYDEYKRFMNYWTSSNEDLDLCRYLGCTLYVFRHPE |
| | VDFIIIINTSPPFLDTEITGPSIHPGMMALNKRSRWIPSIKNRPGRKHYIKI |
| | KVGAPRMFTDKWYPQTDLCDMTLLTIFASAADMQYPFGSPLTDTIVVS |
| | FQVL (SEQ ID NO: 939) |
| Hypervariable | QSMYNDCLSVLPDNFAETSGKGTQLHENIIQHLPYYNTTQTQAQFKRFI |
| domain | ENMNATNGDNIWASYINTTKFSSANTPKNDTGIGGPYTTYSDSWYKGT |
| | VYNDKIKTIPIKASKLYYEQTKNLIGITFTGSTHRLHYCGGLYSSVWLSA |
| | GRSYFETK (SEQ ID NO: 940) |
| N22 | GPYTDITYNPFSDRGEGNMLWIDWLTKNDSVYSKTSSKCLIENLPLWA |
| | SVYGYKEYCSKVTGDTNIEHNCRCVIRSPYTVPQLLDHNNPFRGYVPY |
| | SFNFGNGKMPGGSSLVPIRMRAKWYPTLFHQKEVLEAIAQAGPFAYHS |
| | DIKKVSLGIKYRFKWV (SEQ ID NO: 941) |
| C-terminal | WGGNPVSQQVVRNPCKTTQGSSGNRVPRSIQVVDPRYNTPELTIHAWD |
| domain | FRHGFFGRKAIKRMQEQPIPHDTFSAGFKRSRRDTEALQCSQEEQQKEN |
| | LLFPVQQLKRVPPWETSQESQSEEENSQKQETLSQQLRDQLHKQRLMG |
| | EQLRSLLYQMQRVQQNQHINPMLLPKGLALTSISHNVI (SEQ ID NO: |
| | 942) |

TABLE D9
Exemplary Anellovirus ORF1 amino acid subsequence (Alphatorquevirus)-
Clade 7

| | |
|---|---|
| Name | Ring 7.0 |
| Genus/Clade | Alphatorquevirus-Clade 7 |

Accession Number
Protein Accession Number
Full Sequence: 766 AA
1 10 20 30 40 50
| | | | | |
MAWRWWWQRRWRRRRWPRRRWRRLRRRRPRRPVRRRRRRTTVRRRRWRGR
RGRRTYTRRAVRRRRRPRKRLVLTQWSPQTVRNCSIRGIVPMVICGHTKA
GRNYAIHSEDFTTQIQPFGGSFSTTTWSLKVLWDEHQKFQNRWSYPNTQL
DLARYRGVTFWFYRDQKTDYIVQWSRNPPFKLNKYSSAMYHPGMMMQAKR
KLVVPSFQTRPKGKKRYRVTIKPPNMFADKWYTQEDLCPVPLVQIVVSAA
SLLHPFCPPQTNNPCITFQVLKDIYDECIGVNETMKDKYKKLQTTLYTTC
TYYQTTQVLAQLSPAFQPAMKPTTTQSAATATTLGNYVPELKYNNGSFHT
GQNAVFGMCSYKPTDSIMTKANGWFWQNLMVDNNLHSSYGKATLECMEYH
TGIYSSIFLSPQRSLEFPAAYQDVTYNPNCDRAVGNVVWFQYSTKMDTNF
DETKCKCVLKNIPLWAAFNGYSDFIMQELSISTEIHNFGIVCFQCPYTFP
PCFNKNKPLKGYVFYDTTFGNGKMPDGSGHVPIYWQQRWWIRLAFQVQVM
HDFVLTGPFSYKDDLANTTLTARYKFKFKWGGNIIPEQIIKNPCHREQSL
ASYPDRQRRDLQVVDPSTMGPIYTFHTWDWRRGLFGADAIQRVSQKPGDA
LRFTNPFKRPRYLPPTDREDYRQEEDFALQEKRRRTSTEEAQDEESPPES
APLLQQQQQQRQLSVHLAEQQRLGVQLRYILQEVLKTQAGLHLNPLLLGP
PQTRSISLSPPKAYSP (SEQ ID NO: 943)
Annotations:

| Putative Domain | AA range |
|---|---|
| Arg-Rich Region | 1-70 |
| Jelly-roll domain | 71-271 |
| Hypervariable Region | 272-418 |
| N22 | 419-579 |
| C-terminal Domain | 580-766 |

TABLE D10
Exemplary Anellovirus ORF1 amino acid subsequence (Alphatorquevirus)-Clade 7
Ring7.0 (Alphatorquevirus)

| | |
|---|---|
| Arg-Rich | MAWRWWWQRRWRRRRWPRRRWRRLRRRRPRRPVRRRRRRTTVRRR |
| Region | RWRGRRGRRTYTRRAVRRRRRPRKR (SEQ ID NO: 944) |
| Jelly-roll | LVLTQWSPQTVRNCSIRGIVPMVICGHTKAGRNYAIHSEDFTTQIQPFG |
| Domain | GSFSTTTWSLKVLWDEHQKFQNRWSYPNTQLDLARYRGVTFWFYRD |
| | QKTDYIVQWSRNPPFKLNKYSSAMYHPGMMMQAKRKLVVPSFQTRP |
| | KGKKRYRVTIKPPNMFADKWYTQEDLCPVPLVQIVVSAASLLHPFCPP |
| | QTNNPCITFQVL (SEQ ID NO: 945) |
| Hypervariable | KDIYDECIGVNETMKDKYKKLQTTLYTTCTYYQTTQVLAQLSPAFQPA |
| domain | MKPTTTQSAATATTLGNYVPELKYNNGSFHTGQNAVFGMCSYKPTDSI |
| | MTKANGWFWQNLMVDNNLHSSYGKATLECMEYHTGIYSSIFLSPQRS |
| | LEFP (SEQ ID NO: 946) |
| N22 | AAYQDVTYNPNCDRAVGNVVWFQYSTKMDTNFDETKCKCVLKNIPL |
| | WAAFNGYSDFIMQELSISTEIHNFGIVCFQCPYTFPPCFNKNKPLKGYVF |
| | YDTTFGNGKMPDGSGHVPIYWQQRWWIRLAFQVQVMHDFVLTGPFS |
| | YKDDLANTTLTARYKFKFK (SEQ ID NO: 947) |
| C-terminal | WGGNIIPEQIIKNPCHREQSLASYPDRQRRDLQVVDPSTMGPIYTFHTW |
| domain | DWRRGLFGADAIQRVSQKPGDALRFTNPFKRPRYLPPTDREDYRQEED |
| | FALQEKRRRTSTEEAQDEESPPESAPLLQQQQQQRQLSVHLAEQQRLG |
| | VQLRYILQEVLKTQAGLHLNPLLLGPPQTRSISLSPPKAYSP (SEQ ID |
| | NO: 948) |

TABLE D11
Ring10 ORF1 amino acid subsequence (Betatorquevirus)

| | |
|---|---|
| Arg-Rich Region | MPWWYRRRSYNPWRRRNWFRRPRKTIYRRYRRRRRWVR |
| (1-45) | RKPFYKR |
| Jelly-roll Domain | KIKRLNIVEWQPKSIRKCRIKGMLCLFQTTEDRLSYNFDMY |
| (46-229) | EESIIPEKLPGGGGFSIKNISLYALYQEHIHAHNIFTHTNTDR |
| | PLARYTGCSLKFYQSKDIDYVVTYSTSLPLRSSMGMYNSM |
| | QPSIHLMQQNKLIVPSKQTQKRRKPYIKKHISPPTQMKSQW |
| | YFQHNIANIPLLMIRTTAL |
| Hypervariable domain | TLDNYYIGSRQLSTNVTIHTLNTTYIQNRDWGDRNKTYYC |
| (230-385) | QTLGTQRYFLYGTHSTAQNINDIKLQELIPLTNTQDYVQGF |
| | DWTEKDKHNITTYKEFLTKGAGNPFHAEWITAQNPVIHTA |
| | NSPTQIEQIYTASTTTFQNKKLTDLPTPGYIFITP |
| N22 domain | TVSLRYNPYKDLAERNKCYFVRSKINAHGWDPEQHQELIN |
| (386-543) | SDLPQWLLLFGYPDYIKRTQNFALVDTNYILVDHCPYTNPE |
| | KTPFIPLSTSFIEGRSPYSPSDTHEPDEEDQNRWYPCYQYQQ |
| | ESINSICLSGPGTPKIPKGITAEAKVKYSFNFKWG |
| C-terminal domain | GDLPPMSTITNPTDQPTYVVPNNFNETTSLQNPTTRPEHFLY |
| (544-672) | SFDERRGQLTEKATKRLLKDWETKETSLLSTEYRFAEPTQT |
| | QAPQEDPSSEEEEESNLFERLLRQRTKQLQLKRRIIQTLKDL |
| | QKLE |

### Consensus ORF1 Domain Sequences

In some embodiments, an ORF1 molecule, e.g., as described herein, comprises one or more of a jelly-roll domain, N22 domain, and/or C-terminal domain (CTD). In some embodiments, thejelly-roll domain comprises an amino acid sequence having ajelly-roll domain consensus sequence as described herein (e.g., as listed in any of Tables 37A-37C). In some embodiments, the N22 domain comprises an amino acid sequence having a N22 domain consensus sequence as described herein (e.g., as listed in any of Tables 37A-37C). In some embodiments, the CTD domain comprises an amino acid sequence having a CTD domain consensus sequence as described herein (e.g., as listed in any of Tables 37A-37C). In some embodiments, the amino acids listed in any of Tables 37A-37C in the format “(X_{a-b})” comprise a contiguous series of amino acids, in which the series comprises at least a, and at most b, amino acids. In certain embodiments, all of the amino acids in the series are identical. In other embodiments, the series comprises at least two (e.g., at least 2, 3, 4, 5, 6, 7, 8, 9, 10, 11, 12, 13, 14, 15, 16, 17, 18, 19, 20, or 21) different amino acids.

TABLE 37A
Alphatorquevius ORF1 domain consensus sequences

| Domain | Sequence | SEQ ID NO: |
|---|---|---|
| Jelly-Roll | LVLTQWQPNTVRRCYIRGYLPLIICGEN(X₀₋₃)TTSRNYA | 227 |
| | THSDDTIQKGPFGGGMSTTTFSLRVLYDEYQRFMNRW | |
| | TYSNEDLDLARYLGCKFTFYRHPDXDFIVQYNTNPPFK | |
| | DTKLTAPSIHP(X₁₋₅)GMLMLSKRKILIPSLKTRPKGKHY | |
| | VKVRIGPPKLFEDKWYTQSDLCDVPLVXLYATAADLQ | |
| | HPFGSPQTDNPCVTFQVLGSXYNKHLSISP; | |
| | wherein X = any amino acid. | |
| N22 | SNFEFPGAYTDITYNPLTDKGVGNMVWIQYLTKPDTIX | 228 |
| | DKTQS(X₀₋₃)KCLIEDLPLWAALYGYVDFCEKETGDSAII | |
| | XNXGRVLIRCPYTKPPLYDKT(X₀₋₄)NKGFVPYSTNFGN | |
| | GKMPGGSGYVPIYWRARWYPTLFHQKEVLEDIVQSGP | |
| | FAYKDEKPSTQLVMKYCFNFN; | |
| | wherein X = any amino acid. | |
| CTD | WGGNPISQQVVRNPCKDSG(X₀₋₃)SGXGRQPRSVQVVD | 229 |
| | PKYMGPEYTFHSWDWRRGLFGEKAIKRMSEQPTDDEI | |
| | FTGGXPKRPRRDPPTXQXPEE(X₁₋₄)QKESSSFR(X₂₋₁₄)PW | |
| | ESSSQEXESESQEEEE(X₀₋₃₀)EQTVQQQLRQQLREQRRL | |
| | RVQLQLLFQQLLKT(X₀₋₄)QAGLHINPLLLSQA(X₀₋₄₀)*; | |
| | wherein X = any amino acid. | |

TABLE 37B
Betatorquevius ORF1 domain consensus sequences

| Domain | Sequence | SEQ ID NO: |
|---|---|---|
| Jelly-Roll | LKQWQPSTIRKCKIKGYLPLFQCGKGRISNNYTQYKESI | 230 |
| | VPHHEPGGGGWSIQQFTLGALYEEHLKLRNWWTKSN | |
| | DGLPLVRYLGCTIKLYRSEDTDYIVTYQRCYPMTATKL | |
| | TYLSTQPSRMLMNKHKIIVPSKXT(X₁₋₄)NKKKKPYKKIF | |
| | IKPPSQMQNKWYFQQDIANTPLLQLTXTACSLDRMYL | |
| | SSDSISNNITFTSLNTNFFQNPNFQ; | |
| | wherein X = any amino acid. | |
| N22 | (X₄₋₁₀)TPLYFECRYNPFKDKGTGNKVYLVSNN(X₁₋₈)TG | 231 |
| | WDPPTDPDLIIEGFPLWLLLWGWLDWQKKLGKIQNID | |
| | TDYILVIQSXYYIPP(X₁₋₃)KLPYYVPLDXD(X₀₋₂)FLHGRS | |
| | PY(X₃₋₁₆)PSDKQHWHPKVRFQXETINNIALTGPGTPKLP | |
| | NQKSIQAHMKYKFYFK; | |
| | wherein X = any amino acid. | |
| CTD | WGGCPAPMETITDPCKQPKYPIPNNLLQTTSLQXPTTPI | 232 |
| | ETYLYKFDERRGLLTKKAAKRIKKDXTTETTLFTDTGX | |
| | XTSTTLPTXXQTETTQEEXTSEEE(X₀₋₅)ETLLQQLQQLR | |
| | RKQKQLRXRILQLLQLLXLL(X₀₋₂₆)*; | |
| | wherein X = any amino acid. | |

TABLE 37C
Gammatorquevius ORF1 domain consensus sequences

| Domain | Sequence | SEQ ID NO: |
|---|---|---|
| Jelly-Roll | TIPLKQWQPESIRKCKIKGYGTLVLGAEGRQFYCYTNE | 233 |
| | KDEYTPPKAPGGGGFGVELFSLEYLYEQWKARNNIWT | |
| | KSNXYKDLCRYTGCKITFYRHPTTDFIVXYSRQPPFEID | |
| | KXTYMXXHPQXLLLRKHKKIILSKATNPKGKLKKKIKI | |
| | KPPKQMLNKWFFQKQFAXYGLVQLQAAACBLRYPRL | |
| | GCCNENRLITLYYLN; | |
| | wherein X = any amino acid. | |
| N22 | LPIVVARYNPAXDTGKGNKXWLXSTLNGSXWAPPTTD | 234 |
| | KDLIIEGLPLWLALYGYWSYJKKVKKDKGILQSHMFV | |
| | VKSPAIQPLXTATTQXTFYPXIDNSFIQGKXPYDEPJTX | |
| | NQKKLWYPTLEHQQETINAIVESGPYVPKLDNQKNST | |
| | WELXYXYTFYFK; | |
| | wherein X = any amino acid. | |
| CTD | WGGPQIPDQPVEDPKXQGTYPVPDTXQQTIQIXNPLKQ | 235 |
| | KPETMFHDWDYRRGIITSTALKRMQENLETDSSFXSDS | |
| | EETP(X₀₋₂)KKKKRLTXELPXPQEETEEIQSCLLSLCEEST | |
| | CQEE(X₁₋₆)ENLQQLIHQQQQQQQQLKHNILKLLSDLKZ | |
| | KQRLLQLQTGILE(X₁₋₁₀)* | |
| | wherein X = any amino acid. | |

In some embodiments, the jelly-roll domain comprises a jelly-roll domain amino acid sequence as listed in any of Tables 37A-37C, or an amino acid sequence having at least 70%, 75%, 80%, 8%, 90%, 95%, 96%, 97%, 98%, 99%, or 100% sequence identity thereto. In some embodiments, the N22 domain comprises a N22 domain amino acid sequence as listed in any of Tables 37A-37C, or an amino acid sequence having at least 70%, 75%, 80%, 8%, 90%, 95%, 96%, 97%, 98%, 99%, or 100% sequence identity thereto. In some embodiments, the CTD domain comprises a CTD domain amino acid sequence as listed in any of Tables 37A-37C, or an amino acid sequence having at least 70%, 75%, 80%, 8%, 90%, 95%, 96%, 97%, 98%, 99%, or 100% sequence identity thereto.

### Exemplary ORF1 Variant Constructs

Provided in the table below are exemplary Anellovirus ORF1 mutant and variant sequences.

TABLE X1
Exemplary Anellovirus ORF1 variant constructs

| #ID | Sequence |
|---|---|
| Ring2 | MPYYYRRRRYNYRRPRWYGRGWIRRPFRRRFRRKRRVRPTYTTIPLKQ |
| delCterm | WQPPYKRTCYIKGQDCLIYYSNLRLGMNSTMYEKSIVPVHWPGGGSFS |
| (Δ611-666) | VSMLTLDALYDIHKLCRNWWTSTNQDLPLVRYKGCKITFYQSTFTDYI |
| (7047) | VRIHTELPANSNKLTYPNTHPLMMMMSKYKHIIPSRQTRRKKKPYTKIF |
| | VKPPPQFENKWYFATDLYKIPLLQIHCTACNLQNPFVKPDKLSNNVTL |
| | WSLNTISIQNRNMSVDQGQSWPFKILGTQSFYFYFYTGANLPGDTTQIP |
| | VADLLPLTNPRINRPGQSLNEAKITDHITFTEYKNKFTNYWGNPFNKHI |
| | QEHLDMILYSLKSPEAIKNEWTTENMKWNQLNNAGTMALTPFNEPIFT |
| | QIQYNPDRDTGEDTQLYLLSNATGTGWDPPGIPELILEGFPLWLIYWGF |
| | ADFQKNLKKVTNIDTNYMLVAKTKFTQKPGTFYLVILNDTFVEGNSPY |
| | EKQPLPEDNIKWYPQVQYQLEAQNKLLQTGPFTPNIQGQLSDNISMFYK |
| | FYFKWGGSPPKAINVENPAHQIQYPIPRNEHETTSLQSPGEAPESILYSFD |
| | YRHGNYTTTALSRISQDWALKDTVSK |
| Ring19 | MPWYPRRRYPRRRYRWLRRWRARRPFRPRYRRRYWVRNYSRKRKLF |
| delCterm | KITTKEWQPKVIRKTHVKGTYPLFLCTKHRINNNMIQYLDSIAPEHYYG |
| (Δ600-655) | GGGFSIMQFSLQALYEEFIKAKNWWTNTNCFLPLVRYMGCSFKFYKTE |
| (7120) | FYDYIVLIERCYPLACTDEMYLSTQPSIMMLTRKCIFVPCKQNSKGKKP |
| | YKKVRVRPPSQMTTGWHFSQDLANMPLVVLKTSVCSFDRYYTDSTAK |
| | STTIGFKTLNTQTFRYHDWQEPPTTGYKPQNLLWFYGAENGSPVDPNN |
| | TIVSNLIYLGGTGPYEKGTPIKTNISNYFSEPKLWGNIFHDDYTSGTSPVF |
| | VTNKSPSEIKTAWNTIKDLTVKASGVFTLRTIPLWLPCRYNPFADKATN |
| | NKIWLVSIHSDHTEWKPIDNPLLQRTDLPLWLLVWGWQDWQKKNQQT |
| | SQPDINYLTVISSPYISCYPKLDYYVLLDEGFWEGHSTYIESITDSDKKH |
| | WYPKNRFQIETLNLIANTGPGTVKLRENQAAEGHMVYRFNFKLGGCPA |
| | PMEKICDPSKQSKYPIPNNQQQTTSLQSPENPIQTYLYDFDERRGLLTER |
| | ATKRIKQDHTSEKTVLP |

### Identification of ORF1 Protein Sequences

In some embodiments, an Anellovirus ORF1 protein sequence, or a nucleic acid sequence encoding an ORF1 protein, can be identified from the genome of an Anellovirus (e.g., a putative Anellovirus genome identified, for example, by nucleic acid sequencing techniques, e.g., deep sequencing techniques). In some embodiments, an ORF1 protein sequence is identified by one or more (e.g., 1, 2, or all 3) of the following selection criteria:
(i) Length Selection: Protein sequences (e.g., putative Anellovirus ORF1 sequences passing the criteria described in (ii) or (iii) below) may be size-selected for those greater than about 600 amino acid residues to identify putative Anellovirus ORF1 proteins. In some embodiments, an Anellovirus ORF1 protein sequence is at least about 600, 650, 700, 750, 800, 850, 900, 950, or 1000 amino acid residues in length. In some embodiments, an Alphatorquevirus ORF1 protein sequence is at least about 700, 710, 720, 730, 740, 750, 760, 770, 780, 790, 800, 900, or 1000 amino acid residues in length. In some embodiments, a Betatorquevirus ORF1 protein sequence is at least about 650, 660, 670, 680, 690, 700, 750, 800, 900, or 1000 amino acid residues in length. In some embodiments, a Gammatorquevirus ORF1 protein sequence is at least about 650, 660, 670, 680, 690, 700, 750, 800, 900, or 1000 amino acid residues in length. In some embodiments, a nucleic acid sequence encoding an Anellovirus ORF1 protein is at least about 1800, 1900, 2000, 2100, 2200, 2300, 2400, or 2500 nucleotides in length. In some embodiments, a nucleic acid sequence encoding an Alphatorquevirus ORF1 protein sequence is at least about 2100, 2150, 2200, 2250, 2300, 2400, or 2500 nucleotides in length. In some embodiments, a nucleic acid sequence encoding a Betatorquevirus ORF1 protein sequence is at least about 1900, 1950, 2000, 2500, 2100, 2150, 2200, 2250, 2300, 2400, or 2500 or 1000 nucleotides in length. In some embodiments, a nucleic acid sequence encoding a Gammatorquevirus ORF1 protein sequence is at least about 1900, 1950, 2000, 2500, 2100, 2150, 2200, 2250, 2300, 2400, or 2500 or 1000 nucleotides in length.
(ii) Presence of ORF1 motif Protein sequences (e.g., putative Anellovirus ORF1 sequences passing the criteria described in (i) above or (iii) below) may be filtered to identify those that contain the conserved ORF1 motif in the N22 domain described above. In some embodiments, a putative Anellovirus ORF1 sequence comprises the sequence YNPXXDXGXXN. In some embodiments, a putative Anellovirus ORF1 sequence comprises the sequence Y[NCS]PXXDX[GASKR]XX[NTSVAK].
(iii) Presence of arginine-rich region: Protein sequences (e.g., putative Anellovirus ORF1 sequences passing the criteria described in (i) and/or (ii) above) may be filtered for those that include an arginine-rich region (e.g., as described herein). In some embodiments, a putative Anellovirus ORF1 sequence comprises a contiguous sequence of at least about 30, 35, 40, 45, 50, 55, 60, 65, or 70 amino acids that comprises at least 30% (e.g., at least about 20%, 25%, 30%, 35%, 40%, 45%, or 50%) arginine residues. In some embodiments, a putative Anellovirus ORF1 sequence comprises a contiguous sequence of about 35-40, 40-45, 45-50, 50-55, 55-60, 60-65, or 65-70 amino acids that comprises at least 30% (e.g., at least about 20%, 25%, 30%, 35%, 40%, 45%, or 50%) arginine residues. In some embodiments, the arginine-rich region is positioned at least about 30, 40, 50, 60, 70, or 80 amino acids downstream of the start codon of the putative Anellovirus ORF1 protein. In some embodiments, the arginine-rich region is positioned at least about 50 amino acids downstream of the start codon of the putative Anellovirus ORF1 protein.

In some embodiments, an ORF1 protein is identified in an Anellovirus genome sequence as described in Example 36 of PCT Publication No. WO2020/123816 (incorporated herein by reference in its entirety).

### ORF2 Molecules

In some embodiments, the anellovector or anelloVLP comprises an ORF2 molecule and/or a nucleic acid encoding an ORF2 molecule. Generally, an ORF2 molecule comprises a polypeptide having the structural features and/or activity of an Anellovirus ORF2 protein (e.g., an Anellovirus ORF2 protein as described herein, e.g., as listed in any one of Tables A1-A25), or a functional fragment thereof. In some embodiments, an ORF2 molecule comprises an amino acid sequence having at least 70%, 75%, 80%, 85%, 90%, 95%, 96%, 97%, 98%, 99%, or 100% sequence identity to an Anellovirus ORF2 protein sequence as shown in any one of Tables A1-A25.

In some embodiments, an ORF2 molecule comprises an amino acid sequence having at least 75%, 80%, 85%, 90%, 95%, 96%, 97%, 98%, or 99% sequence identity to an Alphatorquevirus, Betatorquevirus, or Gammatorquevirus ORF2 protein. In some embodiments, an ORF2 molecule (e.g., an ORF2 molecule having at least 75%, 80%, 85%, 90%, 95%, 96%, 97%, 98%, or 99% sequence identity to an Alphatorquevirus ORF2 protein) has a length of 250 or fewer amino acids (e.g., about 150-200 amino acids). In some embodiments, an ORF2 molecule (e.g., an ORF2 molecule having at least 75%, 80%, 85%, 90%, 95%, 96%, 97%, 98%, or 99% sequence identity to a Betatorquevirus ORF2 protein) has a length of about 50-150 amino acids. In some embodiments, an ORF2 molecule (e.g., an ORF2 molecule having at least 75%, 80%, 85%, 90%, 95%, 96%, 97%, 98%, or 99% sequence identity to a Gammatorquevirus ORF2 protein) has a length of about 100-200 amino acids (e.g., about 100-150 amino acids). In some embodiments, the ORF2 molecule comprises a helix-turn-helix motif (e.g., a helix-turn-helix motif comprising two alpha helices flanking a turn region). In some embodiments, the ORF2 molecule does not comprise the amino acid sequence of the ORF2 protein of TTV isolate TA278 or TTV isolate SANBAN. In some embodiments, an ORF2 molecule has protein phosphatase activity. In some embodiments, an ORF2 molecule comprises at least one difference (e.g., a mutation, chemical modification, or epigenetic alteration) relative to a wild-type ORF2 protein, e.g., as described herein (e.g., as shown in any one of Tables A1-A25).

### Conserved ORF2 Motif

In some embodiments, a polypeptide (e.g., an ORF2 molecule) described herein comprises the amino acid sequence [W/F]X⁷HX³CX¹CX⁵H (SEQ ID NO: 949), wherein X” is a contiguous sequence of any n amino acids. In embodiments, X⁷ indicates a contiguous sequence of any seven amino acids. In some embodiments, X³ indicates a contiguous sequence of any three amino acids. In some embodiments, X¹ indicates any single amino acid. In some embodiments, X⁵ indicates a contiguous sequence of any five amino acids. In some embodiments, the [W/F]can be either tryptophan or phenylalanine. In some embodiments, the [W/F]X⁷HX³CX¹CX⁵H (SEQ ID NO: 949) is comprised within the N22 domain of an ORF2 molecule, e.g., as described herein. In some embodiments, a genetic element described herein comprises a nucleic acid sequence (e.g., a nucleic acid sequence encoding an ORF2 molecule, e.g., as described herein) encoding the amino acid sequence [W/F]X⁷HX³CX¹CX⁵H (SEQ ID NO: 949), wherein X” is a contiguous sequence of any n amino acids.

### Genetic Elements

In some embodiments, the anellovector comprises a genetic element. In some embodiments, the genetic element has one or more of the following characteristics: is substantially non-integrating with a host cell's genome, is an episomal nucleic acid, is a single stranded DNA, is circular, is about 1 to 10 kb, exists within the nucleus of the cell, can be bound by endogenous proteins, produces an effector, such as a polypeptide or nucleic acid (e.g., an RNA, iRNA, microRNA) that targets a gene, activity, or function of a host or target cell. In one embodiment, the genetic element is a substantially non-integrating DNA. In some embodiments, the genetic element comprises a packaging signal, e.g., a sequence that binds a capsid protein. In some embodiments, outside of the packaging or capsid-binding sequence, the genetic element has less than 70%, 60%, 50%, 40%, 30%, 20%, 10%, 5% sequence identity to a wild type Anellovirus nucleic acid sequence, e.g., has less than 70%, 60%, 50%, 40%, 30%, 20%, 10%, 5% sequence identity to an Anellovirus nucleic acid sequence, e.g., as described herein. In some embodiments, outside of the packaging or capsid-binding sequence, the genetic element has less than 500, 450, 400, 350, 300, 250, 200, 150, or 100 contiguous nucleotides that are at least 70%, 75%, 80%, 8%, 90%, 95%, 96%, 97%, 98%, 99%, or 100% identical to an Anellovirus nucleic acid sequence. In certain embodiments, the genetic element is a circular, single stranded DNA that comprises a promoter sequence, a sequence encoding a therapeutic effector, and a capsid binding protein.

In some embodiments, the genetic element has at least about 70%, 75%, 80%, 8%, 90%, 95%, 96%, 97%, 98%, 99%, or 100% sequence identity to an Anellovirus nucleic acid sequence, e.g., as described herein (e.g., as described in any one of Tables N1-N25), or a fragment thereof, or encodes an amino acid sequence having at least about 70%, 75%, 80%, 8%, 90%, 95%, 96%, 97%, 98%, 99%, or 100% sequence identity to an Anellovirus amino acid sequence (e.g., as described in any one of Tables A1-A25), or a fragment thereof. In some embodiments, the genetic element comprises a sequence encoding an effector (e.g., an endogenous effector or an exogenous effector, e.g., a payload), e.g., a polypeptide effector (e.g., a protein) or nucleic acid effector (e.g., a non-coding RNA, e.g., a miRNA, siRNA, mRNA, lncRNA, RNA, DNA, an antisense RNA, gRNA).

In some embodiments, the genetic element has a length less than 20 kb (e.g., less than about 19 kb, 18 kb, 17 kb, 16 kb, 15 kb, 14 kb, 13 kb, 12 kb, 11 kb, 10 kb, 9 kb, 8 kb, 7 kb, 6 kb, 5 kb, 4 kb, 3 kb, 2 kb, 1 kb, or less). In some embodiments, the genetic element has, independently or in addition to, a length greater than 1000b (e.g., at least about 1.1 kb, 1.2 kb, 1.3 kb, 1.4 kb, 1.5 kb, 1.6 kb, 1.7 kb, 1.8 kb, 1.9 kb, 2 kb, 2.1 kb, 2.2 kb, 2.3 kb, 2.4 kb, 2.5 kb, 2.6 kb, 2.7 kb, 2.8 kb, 2.9 kb, 3 kb, 3.1 kb, 3.2 kb, 3.3 kb, 3.4 kb, 3.5 kb, 3.6 kb, 3.7 kb, 3.8 kb, 3.9 kb, 4 kb, 4.1 kb, 4.2 kb, 4.3 kb, 4.4 kb, 4.5 kb, 4.6 kb, 4.7 kb, 4.8 kb, 4.9 kb, 5 kb, or greater).

In some embodiments, the genetic element has a length of about 2.5-4.6, 2.8-4.0, 3.0-3.8, or 3.2-3.7 kb.

In some embodiments, the genetic element has a length of about 1.5-2.0, 1.5-2.5, 1.5-3.0, 1.5-3.5, 1.5-3.8, 1.5-3.9, 1.5-4.0, 1.5-4.5, or 1.5-5.0 kb. In some embodiments, the genetic element has a length of about 2.0-2.5, 2.0-3.0, 2.0-3.5, 2.0-3.8, 2.0-3.9, 2.0-4.0, 2.0-4.5, or 2.0-5.0 kb. In some embodiments, the genetic element has a length of about 2.5-3.0, 2.5-3.5, 2.5-3.8, 2.5-3.9, 2.5-4.0, 2.5-4.5, or 2.5-5.0 kb. In some embodiments, the genetic element has a length of about 3.0-5.0, 3.5-5.0, 4.0-5.0, or 4.5-5.0 kb. In some embodiments, the genetic element has a length of about 1.5-2.0, 2.0-2.5, 2.5-3.0, 3.0-3.5, 3.1-3.6, 3.2-3.7, 3.3-3.8, 3.4-3.9, 3.5-4.0, 4.0-4.5, or 4.5-5.0 kb.

In some embodiments, the genetic element comprises one or more of the features described herein, e.g., a sequence encoding a substantially non-pathogenic protein, a protein binding sequence, one or more sequences encoding a regulatory nucleic acid, one or more regulatory sequences, one or more sequences encoding a replication protein, and other sequences. In some embodiments, the substantially non-pathogenic protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences described herein, an Anellovirus amino acid sequence, e.g., as listed in any one of Tables A1-A25.

In some embodiments, the genetic element was produced from a double-stranded circular DNA (e.g., produced by in vitro circularization). In some embodiments, the genetic element was produced by rolling circle replication from the double-stranded circular DNA. In some embodiments, the rolling circle replication occurs in a cell (e.g., a host cell, e.g., a mammalian cell, e.g., a human cell, e.g., a HEK293T cell, an A549 cell, or a Jurkat cell). In some embodiments, the genetic element can be amplified exponentially by rolling circle replication in the cell. In some embodiments, the genetic element can be amplified linearly by rolling circle replication in the cell. In some embodiments, the double-stranded circular DNA or genetic element is capable of yielding at least 2, 4, 8, 16, 32, 64, 128, 256, 518, 1024 or more times the original quantity by rolling circle replication in the cell. In some embodiments, the double-stranded circular DNA was introduced into the cell, e.g., as described herein.

In some embodiments, the double-stranded circular DNA and/or the genetic element does not comprise one or more bacterial plasmid elements (e.g., a bacterial origin of replication or a selectable marker, e.g., a bacterial resistance gene). In some embodiments, the double-stranded circular DNA and/or the genetic element does not comprise a bacterial plasmid backbone.

In one embodiment, the invention includes a genetic element comprising a nucleic acid sequence (e.g., a DNA sequence) encoding (i) a substantially non-pathogenic exterior protein, (ii) an exterior protein binding sequence that binds the genetic element to the substantially non-pathogenic exterior protein, and (iii) a regulatory nucleic acid. In such an embodiment, the genetic element may comprise one or more sequences with at least about 60%, 70% 80%, 85%, 90% 95%, 96%, 97%, 98% and 99% nucleotide sequence identity to any one of the nucleotide sequences to a native viral sequence (e.g., a native Anellovirus sequence, e.g., as described herein).

In some embodiments, a genetic element as described herein comprises a sequence (e.g., a TATA box, cap site, transcriptional start site, 5′ UTR, open reading frame (ORF), poly(A) signal, or GC-rich region sequence) as listed in any of Tables A1, A3, A5, A7, A9, A11, B1-B5, 1, 3, 5, 7, 9, 11, 13, 15, or 17 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

In some embodiments, a genetic element comprises a sequence encoding an effector (e.g., an exogenous effector). In some embodiments, the effector-encoding sequence is inserted into an Anellovirus genome sequence (e.g., as described herein). In some embodiments, the effector-encoding sequence replaces a contiguous sequence (e.g., of at least 5, 10, 15, 20, 25, 30, 40, 50, 60, 70, 80, 90, 100, 200, 300, 400, 500, 600, 700, 800, 900, 1000, or more nucleotides) from the Anellovirus genome sequence. In some embodiments, the effector-encoding sequence replaces a TATA box, cap site, transcriptional start site, 5′ UTR, open reading frame (ORF), poly(A) signal, or GC-rich region sequence, or a portion thereof (e.g., a portion consisting of at least 5, 10, 15, 20, 25, 30, 40, 50, 60, 70, 80, 90, 100, 200, 300, 400, 500, 600, 700, 800, 900, 1000, or more nucleotides) e.g., as listed in any of Tables A1, A3, A5, A7, A9, A11, B1-B5, 1, 3, 5, 7, 9, 11, 13, 15, or 17 of PCT Publication No. WO2020/123816 (incorporated herein by reference in its entirety), or a sequence having at least 70% 80%, 85%, 90% 95%, 96%, 97%, 98% and 99% nucleotide sequence identity thereto.

In some embodiments, the sequence of a first nucleic acid element comprised in a genetic element (e.g., a TATA box, cap site, transcriptional start site, 5′ UTR, open reading frame (ORF), poly(A) signal, or GC-rich region) overlaps with the sequence of a second nucleic acid element (e.g., a TATA box, cap site, transcriptional start site, 5′ UTR, open reading frame (ORF), poly(A) signal, or GC-rich region), e.g., by at least 5, 10, 15, 20, 25, 30, 40, 50, 60, 70, 80, 90, 100, 150, 200, 250, 300, 400, or 500 nucleotides.

In some embodiments, the sequence of a first nucleic acid element comprised in a genetic element (e.g., a TATA box, cap site, transcriptional start site, 5′ UTR, open reading frame (ORF), poly(A) signal, or GC-rich region) does not overlap with the sequence of a second nucleic acid element (e.g., a TATA box, cap site, transcriptional start site, 5′ UTR, open reading frame (ORF), poly(A) signal, or GC-rich region).

### Protein Bindini Sequence

A strategy employed by many viruses is that the viral capsid protein recognizes a specific protein binding sequence in its genome. For example, in viruses with unsegmented genomes, such as the L-A virus of yeast, there is a secondary structure (stem-loop) and a specific sequence at the 5′ end of the genome that are both used to bind the viral capsid protein. However, viruses with segmented genomes, such as Reoviridae, Orthomyxoviridae (influenza), Bunyaviruses and Arenaviruses, need to package each of the genomic segments. Some viruses utilize a complementarity region of the segments to aid the virus in including one of each of the genomic molecules. Other viruses have specific binding sites for each of the different segments. See for example, Curr Opin Struct Biol. 2010 February; 20(1): 114-120; and Journal of Virology (2003), 77(24), 13036-13041.

In some embodiments, the genetic element encodes a protein binding sequence that binds to the substantially non-pathogenic protein. In some embodiments, the protein binding sequence facilitates packaging the genetic element into the proteinaceous exterior. In some embodiments, the protein binding sequence specifically binds an arginine-rich region of the substantially non-pathogenic protein. In some embodiments, the genetic element comprises a protein binding sequence as described in Example 8 of PCT Publication No. WO 2020/123816, incorporated by reference herein in its entirety. In some embodiments, the genetic element comprises a protein binding sequence having at least 70%, 80%, 85%, 90%, 95%, 96%, 97%, 98%, 99%, or 100% sequence identity to a 5′ UTR conserved domain or GC-rich domain of an Anellovirus sequence (e.g., as shown in any one of Tables N1-N25).

In embodiments, the protein binding sequence has at least about 70%, 75%, 80%, 85%, 90%, 95%, 96%, 97%, 98%, 99%, or 100% sequence identity to the Anellovirus 5′ UTR conserved domain nucleotide sequence of any one of Tables N1-N25. 5′ UTR Re gions

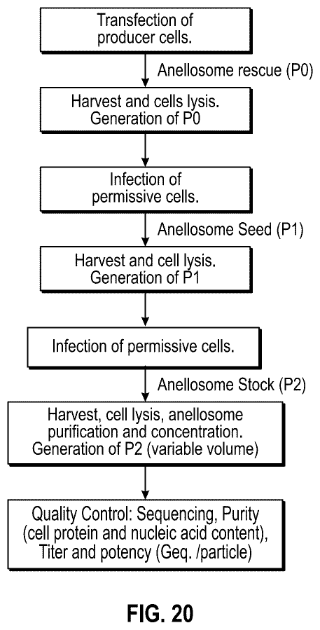

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in Table 38 and/or FIG. 20 of PCT Publication No. WO 2020/123816. In some embodiments, the genetic element (e.g., protein-binding sequence of the genetic element) comprises a nucleic acid sequence of the Consensus 5′ UTR sequence shown in Table 38, wherein X₁, X₂, X₃, X₄, and Xₛ are each independently any nucleotide, e.g., wherein X₁=G or T, X₂=C or A, X₃=G or A, X₄=T or C, and Xs=A, C, or T). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the exemplary TTV 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-CT30F 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-HD23a 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-JA20 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-TJNO2 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the TTV-tth8 5′ UTR sequence shown in Table 38.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Consensus 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 1 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 2 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 3 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 4 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 5 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 6 5′ UTR sequence shown in Table 38. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to the Alphatorquevirus Clade 7 5′ UTR sequence shown in Table 38.

In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of any one of Tables N1-N25.

In some embodiments, the genetic element comprises a nucleic acid sequence having at least about 70%, 75%, 80%, 85%, 90%, 95%, 96%, 97%, 98%, 99%, or 100% sequence identity to the Anellovirus 5′ UTR conserved domain nucleotide sequence of any one of Tables N1-N25.

TABLE 38
Exemplary 5′ UTR sequences from Anelloviruses

| Source | Sequence | SEQ ID NO: |
|---|---|---|
| Consensus | CGGGTGCCGX₁AGGTGAGTTTACACACCGX₂AGT | 105 |
| | CAAGGGGCAATTCGGGCTCX₃GGACTGGCCGGG | |
| | CX₄X₅TGGG | |
| | X₁ = G or T | |
| | X₂ = C or A | |
| | X₃ = Gor A | |
| | X₄ = T or C | |
| | X₅ = A, C, or T | |
| Exemplary TTV | CGGGTGCCGGAGGTGAGTTTACACACCGCAGTC | 106 |
| Sequence | AAGGGGCAATTCGGGCTCGGGACTGGCCGGGCT | |
| | WTGGG | |
| TTV-CT30F | CGGGTGCCGTAGGTGAGTTTACACACCGCAGTC | 107 |
| | AAGGGGCAATTCGGGCTCGGGACTGGCCGGGCT | |
| | ATGGG | |
| TTV-HD23a | CGGGTGCCGGAGGTGAGTTTACACACCGCAGTC | 108 |
| | AAGGGGCAATTCGGGCTCGGGACTGGCCGGGCC | |
| | CTGGG | |
| TTV-JA20 | CGGGTGCCGGAGGTGAGTTTACACACCGCAGTC | 109 |
| | AAGGGGCAATTCGGGCTCGGGACTGGCCGGGCT | |
| | TTGGG | |
| TTV-TJN02 | CGGGTGCCGGAGGTGAGTTTACACACCGCAGTC | 110 |
| | AAGGGGCAATTCGGGCTCGGGACTGGCCGGGCT | |
| | ATGGG | |
| TTV-tth8 | CGGGTGCCGGAGGTGAGTTTACACACCGAAGTC | 111 |
| | AAGGGGCAATTCGGGCTCAGGACTGGCCGGGCT | |
| | TTGGG | |
| Alphatorquevirus | CGGGTGCCGGAGGTGAGTTTACACACCGCAGTC | 112 |
| Consensus 5′ UTR | AAGGGGCAATTCGGGCTCGGGACTGGCCGGGC | |
| | X₁X₂TGGG; wherein X₁ comprises T or C, and | |
| | wherein X₂ comprises A, C, or T. | |
| Alphatorquevirus | CGGGTGCCGTAGGTGAGTTTACACACCGCAGTC | 113 |
| Clade 1 5′ UTR (e.g., | AAGGGGCAATTCGGGCTCGGGACTGGCCGGGCT | |
| TTV-CT30F) | ATGGG | |
| Alphatorquevirus | CGGGTGCCGGAGGTGAGTTTACACACCGCAGTC | 114 |
| Clade 2 5′ UTR (e.g., | AAGGGGCAATTCGGGCTCGGGACTGGCCGGGCC | |
| TTV-P13-1) | CGGG | |
| Alphatorquevirus | CGGGTGCCGGAGGTGAGTTTACACACCGAAGTC | 115 |
| Clade 3 5′ UTR (e.g., | AAGGGGCAATTCGGGCTCAGGACTGGCCGGGCT | |
| TTV-tth8) | TTGGG | |
| Alphatorquevirus | CGGGTGCCGGAGGTGAGTTTACACACCGCAGTC | 116 |
| Clade 4 5′ UTR (e.g., | AAGGGGCAATTCGGGCTCGGGAGGCCGGGCCAT | |
| TTV-HD20a) | GGG | |
| Alphatorquevirus | CGGGTGCCGGAGGTGAGTTTACACACCGCAGTC | 117 |
| Clade 5 5′ UTR (e.g., | AAGGGGCAATTCGGGCTCGGGACTGGCCGGGCC | |
| TTV-16) | CCGGG | |
| Alphatorquevirus | CGGGTGCCGGAGGTGAGTTTACACACCGCAGTC | 118 |
| Clade 6 5′ UTR (e.g., | AAGGGGCAATTCGGGCTCGGGACTGGCCGGGCT | |
| TTV-TJN02) | ATGGG | |
| Alphatorquevirus | CGGGTGCCGAAGGTGAGTTTACACACCGCAGTC | 119 |
| Clade 7 5′ UTR (e.g., | AAGGGGCAATTCGGGCTCGGGACTGGCCGGGCT | |
| TTV-HD16d) | ATGGG | |

Identification of 5′ UTR sequences

In some embodiments, an Anellovirus 5′ UTR sequence can be identified within the genome of an Anellovirus (e.g., a putative Anellovirus genome identified, for example, by nucleic acid sequencing techniques, e.g., deep sequencing techniques). In some embodiments, an Anellovirus 5′ UTR sequence is identified by one or both of the following steps:
(i) Identification of circularization junction point: In some embodiments, a 5′ UTR will be positioned near a circularization junction point of a full-length, circularized Anellovirus genome. A circularization junction point can be identified, for example, by identifying overlapping regions of the sequence. In some embodiments, an overlapping region of the sequence can be trimmed from the sequence to produce a full-length Anellovirus genome sequence that has been circularized. In some embodiments, a genome sequence is circularized in this manner using software. Without wishing to be bound by theory, computationally circularizing a genome may result in the start position for the sequence being oriented in a non-biological. Landmarks within the sequence can be used to re-orient sequences in the proper direction. For example, landmark sequence may include sequences having substantial homology to one or more elements within an Anellovirus genome as described herein (e.g., one or more of a TATA box, cap site, initiator element, transcriptional start site, 5′ UTR conserved domain, ORF1, ORF1/1, ORF1/2, ORF2, ORF2/2, ORF2/3, ORF2t/3, three open-reading frame region, poly(A) signal, or GC-rich region of an Anellovirus, e.g., as described herein).
(ii) Identification of 5′ UTR sequence: Once a putative Anellovirus genome sequence has been obtained, the sequence (or portions thereof, e.g., having a length between about 40-50, 50-60, 60-70, 70-80, 80-90, or 90-100 nucleotides) can be compared to one or more Anellovirus 5′ UTR sequences (e.g., as described herein) to identify sequences having substantial homology thereto. In some embodiments, a putative Anellovirus 5′ UTR region has at least 50%, 60%, 70%, 75%, 80%, 85%, 90%, 95%, 96%, 97%, 98%, 99%, or 100% sequence identity to an Anellovirus 5′ UTR sequence as described herein.

### GC-Rich Reigions

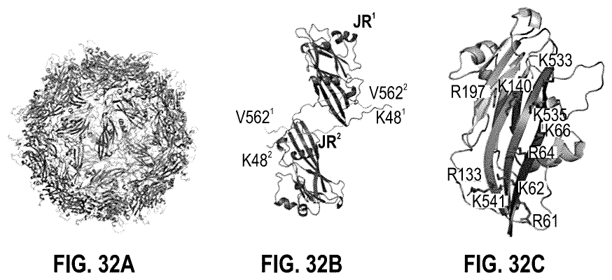

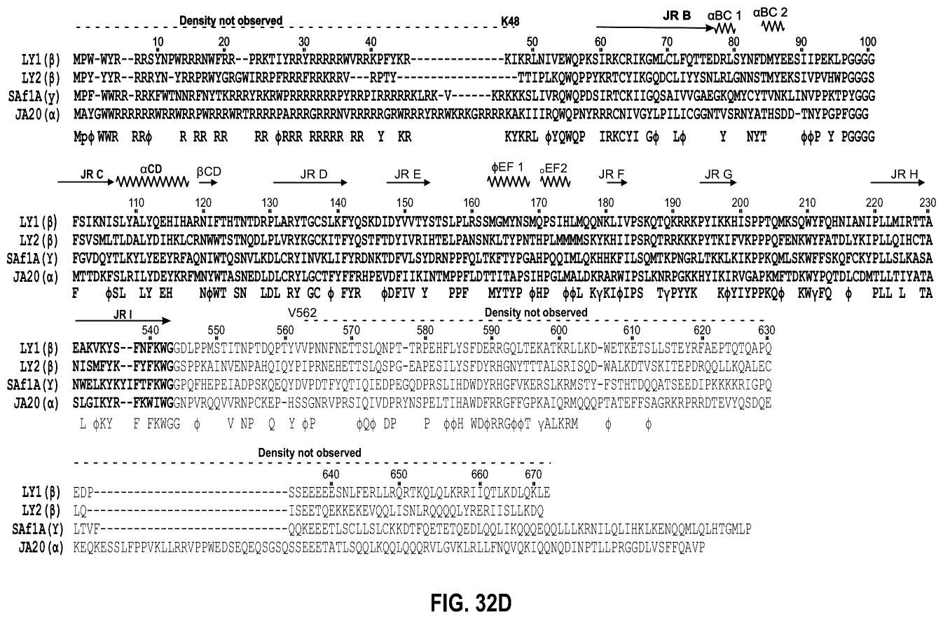

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a nucleic acid sequence shown in any of Table 39 and/or FIGS. 20 and 32 of PCT Publication No. WO 2020/123816. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a GC-rich sequence shown in Table 39.

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region). In some embodiments, the genetic element (e.g., protein-binding sequence of the genetic element) comprises a nucleic acid sequence comprising at least 10, 15, 20, 25, 30, 31, 32, 33, 34, 35, or 36 consecutive nucleotides of a 36-nucleotide GC-rich sequence as shown in Table 39 (e.g., 36-nucleotide consensus GC-rich region sequence 1, 36-nucleotide consensus GC-rich region sequence 2, TTV Clade 1 36-nucleotide region, TTV Clade 3 36-nucleotide region, TTV Clade 3 isolate GH1 36-nucleotide region, TTV Clade 3 sle1932 36-nucleotide region, TTV Clade 4 ctdc002 36-nucleotide region, TTV Clade 5 36-nucleotide region, TTV Clade 6 36-nucleotide region, or TTV Clade 7 36-nucleotide region).

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an Alphatorquevirus GC-rich region sequence, e.g., selected from TTV-CT30F, TTV-P13-1, TTV-tth8, TTV-HD20a, TTV-16, TTV-TJNO2, or TTV-HD16d, e.g., as listed in Table 39. In some embodiments, the genetic element (e.g., protein-binding sequence of the genetic element) comprises a nucleic acid sequence comprising at least 10, 15, 20, 25, 30, 35, 40, 45, 50, 60, 70, 80, 90, 100, 104, 10⁵, 108, 110, 111, 115, 120, 122, 130, 140, 145, 150, 155, or 156 consecutive nucleotides of an Alphatorquevirus GC-rich region sequence, e.g., selected from TTV-CT30F, TTV-P13-1, TTV-tth8, TTV-HD20a, TTV-16, TTV-TJNO2, or TTV-HD16d, e.g., as listed in Table 39.

In some embodiments, the 36-nucleotide GC-rich sequence is selected from: (i) CGCGCTGCGCGCGCCGCCCAGTAGGGGGAGCCATGC (SEQ ID NO: 160),

(SEQ ID NO: 160)
(i) CGCGCTGCGCGCGCCGCCCAGTAGGGGGAGCCATGC,
(SEQ ID NO: 164)
(ii) GCGCTX₁CGCGCGCGCGCCGGGGGGCTGCGCCCCCCC,
wherein X₁ is selected from T, G, or A;
(SEQ ID NO: 165)
(iii) GCGCTTCGCGCGCCGCCCACTAGGGGGCGTTGCGCG;
(SEQ ID NO: 166)
(iv) GCGCTGCGCGCGCCGCCCAGTAGGGGGCGCAATGCG;
(SEQ ID NO: 167)
(v) GCGCTGCGCGCGCGGCCCCCGGGGGAGGCATTGCCT;
(SEQ ID NO: 168)
(vi) GCGCTGCGCGCGCGCGCCGGGGGGGCGCCAGCGCCC;
(SEQ ID NO: 169)
(vii) GCGCTTCGCGCGCGCGCCGGGGGGCTCCGCCCCCCC;
(SEQ ID NO: 170)
(viii) GCGCTTCGCGCGCGCGCCGGGGGGCTGCGCCCCCCC;
(SEQ ID NO: 171)
(ix) GCGCTACGCGCGCGCGCCGGGGGGCTGCGCCCCCCC;
or
(SEQ ID NO: 172)
(x) GCGCTACGCGCGCGCGCCGGGGGGCTCTGCCCCCCC.

In some embodiments, the genetic element (e.g., protein-binding sequence of the genetic element) comprises the nucleic acid sequence CGCGCTGCGCGCGCCGCCCAGTAGGGGGAGCCATGC (SEQ ID NO: 160).

In some embodiments, the genetic element (e.g., protein-binding sequence of the genetic element) comprises a nucleic acid sequence of the Consensus GC-rich sequence shown in Table 39, wherein X₁, X₄, X₅, X₆, X₇, X₁₂, X₁₃, X₁₄, X₁₅, X₂₀, X₂₁, X₂₂, X₂₆, X₂₉, X₃0, and X₃₃ are each independently any nucleotide and wherein X₂, X₃, Xg, X₉, X₁₀, X>>, X₁₆, X₁₇, X₁₈, X₁₉, X₂₃, X₂₄, X₂₅, X₂₇, X₂₈, X₃₁, X₃₂, and X₃₄ are each independently absent or any nucleotide. In some embodiments, one or more of (e.g., all of) X₁ through X₃₄ are each independently the nucleotide (or absent) specified in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to an exemplary TTV GC-rich sequence shown in Table 39 (e.g., the full sequence, Fragment 1, Fragment 2, Fragment 3, or any combination thereof, e.g., Fragments 1-3 in order).

In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-CT30F GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-7 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-HD23a GC-rich sequence shown in Table 39 (e.g., the full sequence, Fragment 1, Fragment 2, Fragment 3, Fragment 4, Fragment 5, Fragment 6, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-JA20 GC-rich sequence shown in Table 39 (e.g., the full sequence, Fragment 1, Fragment 2, or any combination thereof, e.g., Fragments 1 and 2 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-TJNO2 GC-rich sequence shown in Table 39 (e.g., the full sequence, Fragment 1, Fragment 2, Fragment 3, Fragment 4, Fragment 5, Fragment 6, Fragment 7, Fragment 8, or any combination thereof, e.g., Fragments 1-8 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to a TTV-tth8 GC-rich sequence shown in Table 39 (e.g., the full sequence, Fragment 1, Fragment 2, Fragment 3, Fragment 4, Fragment 5, Fragment 6, Fragment 7, Fragment 8, Fragment 9, or any combination thereof, e.g., Fragments 1-6 in order). In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 7 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 8 shown in Table 39. In some embodiments, the genetic element (e.g., protein-binding sequence of the genetic element) comprises a nucleic acid sequence having at least about 75% (e.g., at least 75%, 80%, 85%, 90%, 95%, 96%, 97%, 98%, 99%, or 100%) identity to Fragment 9 shown in Table 39.

TABLE 39
Exemplary GC-rich sequences from Anelloviruses

| | | | SEQ ID |
|---|---|---|---|
| Source | | Sequence | NO: |
| Consensus | | CGGCGGX₁GGX₂GX₃X₄X₅CGCGCTX₆CGCGC | 120 |
| | | GCX₇X₈X₉X₁₀CX₁₁X₁₂X₁₃X₁₄GGGGX₁₅X₁₆X₁₇X₁₈ | |
| | | X₁₉X₂₀X₂₁GCX₂₂X₂₃X₂₄X₂₅CCCCCCCX₂₆CGCGC | |
| | | ATX₂₇X₂₈GCX₂₉CGGGX₃₀CCCCCCCCCX₃₁X₃₂X₃₃ | |
| | | GGGGGGCTCCGX₃₄CCCCCCGGCCCCCC | |
| | | X₁ = G or C | |
| | | X₂ = G, C, or absent | |
| | | X₃ = C or absent | |
| | | X₄ = G or C | |
| | | X₅ = G or C | |
| | | X₆ = T, G, or A | |
| | | X₇ = G or C | |
| | | X₈ = G or absent | |
| | | X₉ = C or absent | |
| | | X₁₀ = C or absent | |
| | | X₁₁ = G, A, or absent | |
| | | X₁₂ = G or C | |
| | | X₁₃ = C or T | |
| | | X₁₄ = G or A | |
| | | X₁₅ = G or A | |
| | | X₁₆ = A, G, T, or absent | |
| | | X₁₇ = G, C, or absent | |
| | | X₁₈ = G, C, or absent | |
| | | X₁₉ = C, A, or absent | |
| | | X₂₀ = C or A | |
| | | X₂₁ = T or A | |
| | | X₂₂ = G or C | |
| | | X₂₃ = G, T, or absent | |
| | | X₂₄ = C or absent | |
| | | X₂₅ = G, C, or absent | |
| | | X₂₆ = G or C | |
| | | X₂₇ = G or absent | |
| | | X₂₈ = C or absent | |
| | | X₂₉ = G or A | |
| | | X₃₀ = G or T | |
| | | X₃₁ = C, T, or absent | |
| | | X₃₂ = G, C, A, or absent | |
| | | X₃₃ = G or C | |
| | | X₃₄ = C or absent | |
| Exemplary TTV | Full sequence | GCCGCCGCGGCGGCGGSGGNGNSGCGCGCT | 121 |
| Sequence | | DCGCGCGCSNNNCRCCRGGGGGNNNNCWG | |
| | | CSNCNCCCCCCCCCGCGCATGCGCGGGKCC | |
| | | CCCCCCCNNCGGGGGGCTCCGCCCCCCGGC | |
| | | CCCCCCCCGTGCTAAACCCACCGCGCATGC | |
| | | GCGACCACGCCCCCGCCGCC | |
| | Fragment 1 | GCCGCCGCGGCGGCGGSGGNGNSGCGCGCT | 122 |
| | | DCGCGCGCSNNNCRCCRGGGGGNNNNCWG | |
| | | CSNCNCCCCCCCCCGCGCAT | |
| | Fragment 2 | GCGCGGGKCCCCCCCCCNNCGGGGGGCTC | 123 |
| | | CG | |
| | Fragment 3 | CCCCCCGGCCCCCCCCCGTGCTAAACCCAC | 124 |
| | | CGCGCATGCGCGACCACGCCCCCGCCGCC | |
| TTV-CT30F | Full sequence | GCGGCGG-GGGGGCG-GCCGCG- | 125 |
| | | TTCGCGCGCCGCCCACCAGGGGGTG-- | |
| | | CTGCG-CGCCCCCCCCCGCGCAT | |
| | | GCGCGGGGCCCCCCCCC-- | |
| | | GGGGGGGCTCCGCCCCCCCGGCCCCCCCCC | |
| | | GTGCTAAACCCACCGCGCATGCGCGACCAC | |
| | | GCCCCCGCCGCC | |
| | Fragment 1 | GCGGCGG | 126 |
| | Fragment 2 | GGGGGCG | 127 |
| | Fragment 3 | GCCGCG | 128 |
| | Fragment 4 | TTCGCGCGCCGCCCACCAGGGGGTG | 129 |
| | Fragment 5 | CTGCG | 130 |
| | Fragment 6 | CGCCCCCCCCCGCGCAT | 131 |
| | Fragment 7 | GCGCGGGGCCCCCCCCC | 132 |
| | Fragment 8 | GGGGGGGCTCCGCCCCCCCGGCCCCCCCCC | 133 |
| | | GTGCTAAACCCACCGCGCATGCGCGACCAC | |
| | | GCCCCCGCCGCC | |
| TTV-HD23a | Full sequence | CGGCGGCGGCGGCG- | 134 |
| | | CGCGCGCTGCGCGCGCG--- | |
| | | CGCCGGGGGGGCGCCAGCG- | |
| | | CCCCCCCCCCCGCGCAT | |
| | | GCACGGGTCCCCCCCCCCACGGGGGGCTCC | |
| | | G CCCCCCGGCCCCCCCCC | |
| | Fragment 1 | CGGCGGCGGCGGCG | 135 |
| | Fragment 2 | CGCGCGCTGCGCGCGCG | 136 |
| | Fragment 3 | CGCCGGGGGGGCGCCAGCG | 137 |
| | Fragment 4 | CCCCCCCCCCCGCGCAT | 138 |
| | Fragment 5 | GCACGGGTCCCCCCCCCCACGGGGGGCTCC | 139 |
| | | G | |
| | Fragment 6 | CCCCCCGGCCCCCCCCC | 140 |
| TTV-JA20 | Full sequence | CCGTCGGCGGGGGGGCCGCGCGCTGCGCG | 141 |
| | | CGCGGCCC- | |
| | | CCGGGGGAGGCACAGCCTCCCCCCCCCGCG | |
| | | CGCATGCGCGCGGGTCCCCCCCCCTCCGGG | |
| | | GGGCTCCGCCCCCCGGCCCCCCCC | |
| | Fragment 1 | CCGTCGGCGGGGGGGCCGCGCGCTGCGCG | 142 |
| | | CGCGGCCC | |
| | Fragment 2 | CCGGGGGAGGCACAGCCTCCCCCCCCCGCG | 143 |
| | | CGCATGCGCGCGGGTCCCCCCCCCTCCGGG | |
| | | GGGCTCCGCCCCCCGGCCCCCCCC | |
| TTV-TJN02 | Full sequence | CGGCGGCGGCG-CGCGCGCTACGCGCGCG-- | 144 |
| | | -CGCCGGGGGG----CTGCCGC- | |
| | | CCCCCCCCCGCGCAT | |
| | | GCGCGGGGCCCCCCCCC- | |
| | | GCGGGGGGCTCCG CCCCCCGGCCCCCC | |
| | Fragment 1 | CGGCGGCGGCG | 145 |
| | Fragment 2 | CGCGCGCTACGCGCGCG | 146 |
| | Fragment 3 | CGCCGGGGGG | 147 |
| | Fragment 4 | CTGCCGC | 148 |
| | Fragment 5 | CCCCCCCCCGCGCAT | 149 |
| | Fragment 6 | GCGCGGGGCCCCCCCCC | 150 |
| | Fragment 7 | GCGGGGGGCTCCG | 151 |
| | Fragment 8 | CCCCCCGGCCCCCC | 152 |
| TTV-tth8 | Full sequence | GCCGCCGCGGCGGCGGGGG- | 153 |
| | | GCGGCGCGCTGCGCGCGCCGCCCAGTAGG | |
| | | GGGAGCCATGCG---CCCCCCCCCGCGCAT | |
| | | GCGCGGGGCCCCCCCCC- | |
| | | GCGGGGGGCTCCG | |
| | | CCCCCCGGCCCCCCCCG | |
| | Fragment 1 | GCCGCCGCGGCGGCGGGGG | 154 |
| | Fragment 2 | GCGGCGCGCTGCGCGCGCCGCCCAGTAGG | 155 |
| | | GGGAGCCATGCG | |
| | Fragment 3 | CCCCCCCCCGCGCAT | 156 |
| | Fragment 4 | GCGCGGGGCCCCCCCCC | 157 |
| | Fragment 5 | GCGGGGGGCTCCG | 158 |
| | Fragment 6 | CCCCCCGGCCCCCCCCG | 159 |
| | Fragment 7 | CGCGCTGCGCGCGCCGCCCAGTAGGGGGA | 160 |
| | | GCCATGC | |
| | Fragment 8 | CCGCCATCTTAAGTAGTTGAGGCGGACGGT | 161 |
| | | GGCGTGAGTTCAAAGGTCACCATCAGCCAC | |
| | | ACCTACTCAAAATGGTGG | |
| | Fragment 9 | CTTAAGTAGTTGAGGCGGACGGTGGCGTGA | 162 |
| | | GTTCAAAGGTCACCATCAGCCACACCTACT | |
| | | CAAAATGGTGGACAATTTCTTCCGGGTCAA | |
| | | AGGTTACAGCCGCCATGTTAAAACACGTGA | |
| | | CGTATGACGTCACGGCCGCCATTTTGTGAC | |
| | | ACAAGATGGCCGACTTCCTTCC | |
| Additional GC-rich | 36-nucleotide | CGCGCTGCGCGCGCCGCCCAGTAGGGGGA | 163 |
| Sequences (as shown | consensus GC- | GCCATGC | |
| in FIG. 32 of PCT | rich region | | |
| Publication No. | sequence 1 | | |
| WO 2020/123816) | 36-nucleotide | GCGCTX₁CGCGCGCGCGCCGGGGGGCTGCG | 164 |
| | region | CCCCCCC, wherein X₁ is selected from T, G, or A | |
| | consensus | | |
| | sequence 2 | | |
| | TTV Clade 1 | GCGCTTCGCGCGCCGCCCACTAGGGGGCGT | 165 |
| | 36-nucleotide | TGCGCG | |
| | region | | |
| | TTV Clade 3 | GCGCTGCGCGCGCCGCCCAGTAGGGGGCG | 166 |
| | 36-nucleotide | CAATGCG | |
| | region | | |
| | TTV Clade 3 | GCGCTGCGCGCGCGGCCCCCGGGGGAGGC | 167 |
| | isolate GH1 36- | ATTGCCT | |
| | nucleotide | | |
| | region | | |
| | TTV Clade 3 | GCGCTGCGCGCGCGCGCCGGGGGGGCGCC | 168 |
| | sle 1932 36- | AGCGCCC | |
| | nucleotide | | |
| | region | | |
| | TTV Clade 4 | GCGCTTCGCGCGCGCGCCGGGGGGCTCCGC | 169 |
| | ctdc002 36- | CCCCCC | |
| | nucleotide | | |
| | region | | |
| | TTV Clade 5 | GCGCTTCGCGCGCGCGCCGGGGGGCTGCGC | 170 |
| | 36-nucleotide | CCCCCC | |
| | region | | |
| | TTV Clade 6 | GCGCTACGCGCGCGCGCCGGGGGGCTGCG | 171 |
| | 36-nucleotide | CCCCCCC | |
| | region | | |
| | TTV Clade 7 | GCGCTACGCGCGCGCGCCGGGGGGCTCTGC | 172 |
| | 36-nucleotide | CCCCCC | |
| | region | | |
| Additional | TTV-CT30F | GCGGCGGGGGGGCGGCCGCGTTCGCGCGC | 801 |
| Alphatorquevirus | | CGCCCACCAGGGGGTGCTGCGCGCCCCCCC | |
| GC-rich region | | CCGCGCATGCGCGGGGCCCCCCCCCGGGG | |
| sequences | | GGGCTCCGCCCCCCCGGCCCCCCCCCGTGC | |
| | | TAAACCCACCGCGCATGCGCGACCACGCCC | |
| | | CCGCCGCC | |
| | TTV-P13-1 | CCGAGCGTTAGCGAGGAGTGCGACCCTACC | 802 |
| | | CCCTGGGCCCACTTCTTCGGAGCCGCGCGC | |
| | | TACGCCTTCGGCTGCGCGCGGCACCTCAGA | |
| | | CCCCCGCTCGTGCTGACACGCTTGCGCGTG | |
| | | TCAGACCACTTCGGGCTCGCGGGGGTCGGG | |
| | TTV-tth8 | GCCGCCGCGGCGGCGGGGGGCGGCGCGCT | 803 |
| | | GCGCGCGCCGCCCAGTAGGGGGAGCCATG | |
| | | CGCCCCCCCCCGCGCATGCGCGGGGCCCCC | |
| | | CCCCGCGGGGGGCTCCGCCCCCCGGCCCCC | |
| | | CCCG | |
| | TTV-HD20a | CGGCCCAGCGGCGGCGCGCGCGCTTCGCGC | 804 |
| | | GCGCGCCGGGGGGCTCCGCCCCCCCCCGCG | |
| | | CATGCGCGGGGCCCCCCCCCGCGGGGGGCT | |
| | | CCGCCCCCCGGTCCCCCCCCG | |
| | TTV-16 | CGGCCGTGCGGCGGCGCGCGCGCTTCGCGC | 805 |
| | | GCGCGCCGGGGGCTGCCGCCCCCCCCCGCG | |
| | | CATGCGCGCGGGGCCCCCCCCCGCGGGGG | |
| | | GCTCCGCCCCCCGGCCCCCCCCCCCG | |
| | TTV-TJN02 | CGGCGGCGGCGCGCGCGCTACGCGCGCGC | 806 |
| | | GCCGGGGGGCTGCCGCCCCCCCCCCGCGCA | |
| | | TGCGCGGGGCCCCCCCCCGCGGGGGGCTCC | |
| | | GCCCCCCGGCCCCCC | |
| | TTV-HD16d | GGCGGCGGCGCGCGCGCTACGCGCGCGCG | 807 |
| | | CCGGGGAGCTCTGCCCCCCCCCGCGCATGC | |
| | | GCGCGGGTCCCCCCCCCGCGGGGGGCTCCG | |
| | | CCCCCCGGTCCCCCCCCCG | |

In some embodiments, the genetic element comprises a nucleic acid sequence having at least about 70%, 75%, 80%, 85%, 90%, 95%, 96%, 97%, 98%, 99%, or 100% sequence identity to the Anellovirus GC-rich nucleotide sequence of any one of Tables N1-N25.

### Effector

In some embodiments, the genetic element may include one or more sequences that encode a functional effector, e.g., an endogenous effector or an exogenous effector, e.g., a therapeutic polypeptide or nucleic acid, e.g., cytotoxic or cytolytic RNA or protein. In some embodiments, the functional nucleic acid is a non-coding RNA. In some embodiments, the functional nucleic acid is a coding RNA. The effector may modulate a biological activity, for example increasing or decreasing enzymatic activity, gene expression, cell signaling, and cellular or organ function. Effector activities may also include binding regulatory proteins to modulate activity of the regulator, such as transcription or translation. Effector activities also may include activator or inhibitor functions. For example, the effector may induce enzymatic activity by triggering increased substrate affinity in an enzyme, e.g., fructose 2,6-bisphosphate activates phosphofructokinase 1 and increases the rate of glycolysis in response to the insulin. In another example, the effector may inhibit substrate binding to a receptor and inhibit its activation, e.g., naltrexone and naloxone bind opioid receptors without activating them and block the receptors' ability to bind opioids. Effector activities may also include modulating protein stability/degradation and/or transcript stability/degradation. For example, proteins may be targeted for degradation by the polypeptide co-factor, ubiquitin, onto proteins to mark them for degradation. In another example, the effector inhibits enzymatic activity by blocking the enzyme's active site, e.g., methotrexate is a structural analog of tetrahydrofolate, a coenzyme for the enzyme dihydrofolate reductase that binds to dihydrofolate reductase 1000-fold more tightly than the natural substrate and inhibits nucleotide base synthesis.

In some embodiments, the sequence encoding an effector is part of the genetic element, e.g., it can be inserted at an insert site as described in Example 10, 12, or 22 of PCT Publication No. WO 2020/123816, incorporated by reference herein in its entirety, and Example 28 herein. In some embodiments, the sequence encoding an effector is inserted into the genetic element at a noncoding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the sequence encoding an effector is inserted into the genetic element at about nucleotide 3588 of a TTV-tth8 plasmid, e.g., as described herein or at about nucleotide 2843 of a TTMV-LY2 plasmid, e.g., as described herein. In some embodiments, the sequence encoding an effector is inserted into the genetic element at or within nucleotides 336-3015 of a TTV-tth8 plasmid, e.g., as described herein, or at or within nucleotides 242-2812 of a TTV-LY2 plasmid, e.g., as described herein. In some embodiments, the sequence encoding an effector replaces part or all of an open reading frame (e.g., an ORF as described herein, e.g., an ORF1, ORF1/1, ORF1/2, ORF2, ORF2/2, ORF2/3, and/or ORF2t/3 as shown in any one of Tables A1-A25 or N1-N25).

In some embodiments, the sequence encoding an effector comprises 100-2000, 100-1000, 100-500, 100-200, 200-2000, 200-1000, 200-500, 500-1000, 500-2000, or 1000-2000 nucleotides. In some embodiments, the effector is a nucleic acid or protein payload, e.g., as described in Example 28.

### Regulatory Nucleic Acid

In some embodiments, the effector is a regulatory nucleic acid. Regulatory nucleic acids modify expression of an endogenous gene and/or an exogenous gene. In one embodiment, the regulatory nucleic acid targets a host gene. The regulatory nucleic acids may include, but are not limited to, a nucleic acid that hybridizes to an endogenous gene (e.g., miRNA, siRNA, mRNA, lncRNA, RNA, DNA, an antisense RNA, gRNA as described herein elsewhere), nucleic acid that hybridizes to an exogenous nucleic acid such as a viral DNA or RNA, nucleic acid that hybridizes to an RNA, nucleic acid that interferes with gene transcription, nucleic acid that interferes with RNA translation, nucleic acid that stabilizes RNA or destabilizes RNA such as through targeting for degradation, and nucleic acid that modulates a DNA or RNA binding factor. In some embodiments, the regulatory nucleic acid encodes an miRNA.

In some embodiments, the regulatory nucleic acid comprises RNA or RNA-like structures typically containing 5-500 base pairs (depending on the specific RNA structure, e.g., miRNA 5-30 bps, lncRNA 200-500 bps) and may have a nucleobase sequence identical (or complementary) or nearly identical (or substantially complementary) to a coding sequence in an expressed target gene within the cell, or a sequence encoding an expressed target gene within the cell.

In some embodiments, the regulatory nucleic acid comprises a nucleic acid sequence, e.g., a guide RNA (gRNA). In some embodiments, the DNA targeting moiety comprises a guide RNA or nucleic acid encoding the guide RNA. A gRNA short synthetic RNA can be composed of a “scaffold” sequence necessary for binding to the incomplete effector moiety and a user-defined ˜20 nucleotide targeting sequence for a genomic target. In practice, guide RNA sequences are generally designed to have a length of between 17-24 nucleotides (e.g., 19, 20, or 21 nucleotides) and complementary to the targeted nucleic acid sequence. Custom gRNA generators and algorithms are available commercially for use in the design of effective guide RNAs. Gene editing has also been achieved using a chimeric “single guide RNA” (“sgRNA”), an engineered (synthetic) single RNA molecule that mimics a naturally occurring crRNA-tracrRNA complex and contains both a tracrRNA (for binding the nuclease) and at least one crRNA (to guide the nuclease to the sequence targeted for editing). Chemically modified sgRNAs have also been demonstrated to be effective in genome editing; see, for example, Hendel et al. (2015) Nature Biotechnol., 985-991.

The regulatory nucleic acid comprises a gRNA that recognizes specific DNA sequences (e.g., sequences adjacent to or within a promoter, enhancer, silencer, or repressor of a gene).

Certain regulatory nucleic acids can inhibit gene expression through the biological process of RNA interference (RNAi). RNAi molecules comprise RNA or RNA-like structures typically containing 15-50 base pairs (such as about 18-25 base pairs) and having a nucleobase sequence identical (complementary) or nearly identical (substantially complementary) to a coding sequence in an expressed target gene within the cell. RNAi molecules include, but are not limited to: short interfering RNAs (siRNAs), double-strand RNAs (dsRNA), micro RNAs (miRNAs), short hairpin RNAs (shRNA), meroduplexes, and dicer substrates (U.S. Pat. Nos. 8,084,599 8,349,809 and 8,513,207).

Long non-coding RNAs (lncRNA) are defined as non-protein coding transcripts longer than 100 nucleotides. This somewhat arbitrary limit distinguishes lncRNAs from small regulatory RNAs such as microRNAs (miRNAs), short interfering RNAs (siRNAs), and other short RNAs. In general, the majority (˜78%) of lncRNAs are characterized as tissue-specific. Divergent lncRNAs that are transcribed in the opposite direction to nearby protein-coding genes (comprise a significant proportion ˜20% of total lncRNAs in mammalian genomes) may possibly regulate the transcription of the nearby gene.

The genetic element may encode regulatory nucleic acids with a sequence substantially complementary, or fully complementary, to all or a fragment of an endogenous gene or gene product (e.g., mRNA). The regulatory nucleic acids may complement sequences at the boundary between introns and exons to prevent the maturation of newly-generated nuclear RNA transcripts of specific genes into mRNA for transcription. The regulatory nucleic acids that are complementary to specific genes can hybridize with the mRNA for that gene and prevent its translation. The antisense regulatory nucleic acid can be DNA, RNA, or a derivative or hybrid thereof.

The length of the regulatory nucleic acid that hybridizes to the transcript of interest may be between 5 to 30 nucleotides, between about 10 to 30 nucleotides, or about 11, 12, 13, 14, 15, 16, 17, 18, 19, 20, 21, 22, 23, 24, 25, 26, 27, 28, 29, 30 or more nucleotides. The degree of identity of the regulatory nucleic acid to the targeted transcript should be at least 75%, at least 80%, at least 85%, at least 90%, or at least 95%.

The genetic element may encode a regulatory nucleic acid, e.g., a micro RNA (miRNA) molecule identical to about 5 to about 25 contiguous nucleotides of a target gene. In some embodiments, the miRNA sequence targets a mRNA and commences with the dinucleotide AA, comprises a GC-content of about 30-70% (about 30-60%, about 40-60%, or about 45%-55%), and does not have a high percentage identity to any nucleotide sequence other than the target in the genome of the mammal in which it is to be introduced, for example as determined by standard BLAST search.

In some embodiments, the regulatory nucleic acid is at least one miRNA, e.g., 2, 3, 4, 5, 6, or more. In some embodiments, the genetic element comprises a sequence that encodes an miRNA at least about 75%, 80%, 85%, 90% 95%, 96%, 97%, 98%, 99% or 100% nucleotide sequence identity to any one of the nucleotide sequences or a sequence that is complementary to a sequence described herein. siRNAs and shRNAs resemble intermediates in the processing pathway of the endogenous microRNA (miRNA) genes (Bartel, Cell 116:281-297, 2004). In some embodiments, siRNAs can function as miRNAs and vice versa (Zeng et al., Mol Cell 9:1327-1333, 2002; Doench et al., Genes Dev 17:438-442, 2003). MicroRNAs, like siRNAs, use RISC to downregulate target genes, but unlike siRNAs, most animal miRNAs do not cleave the mRNA. Instead, miRNAs reduce protein output through translational suppression or polyA removal and mRNA degradation (Wu et al., Proc Natl Acad Sci USA 103:4034-4039, 2006). Known miRNA binding sites are within mRNA 3′ UTRs; miRNAs seem to target sites with near-perfect complementarity to nucleotides 2-8 from the miRNA's 5′ end (Rajewsky, Nat Genet 38 Suppl:S8-13, 2006; Lim et al., Nature 433:769-773, 2005). This region is known as the seed region. Because siRNAs and miRNAs are interchangeable, exogenous siRNAs downregulate mRNAs with seed complementarity to the siRNA (Birmingham et al., Nat Methods 3:199-204, 2006. Multiple target sites within a 3′ UTR give stronger downregulation (Doench et al., Genes Dev 17:438-442, 2003).

Lists of known miRNA sequences can be found in databases maintained by research organizations, such as Wellcome Trust Sanger Institute, Penn Center for Bioinformatics, Memorial Sloan Kettering Cancer Center, and European Molecule Biology Laboratory, among others. Known effective siRNA sequences and cognate binding sites are also well represented in the relevant literature. RNAi molecules are readily designed and produced by technologies known in the art. In addition, there are computational tools that increase the chance of finding effective and specific sequence motifs (Lagana et al., Methods Mol. Bio., 2015, 1269:393-412).

The regulatory nucleic acid may modulate expression of RNA encoded by a gene. Because multiple genes can share some degree of sequence homology with each other, in some embodiments, the regulatory nucleic acid can be designed to target a class of genes with sufficient sequence homology. In some embodiments, the regulatory nucleic acid can contain a sequence that has complementarity to sequences that are shared amongst different gene targets or are unique for a specific gene target. In some embodiments, the regulatory nucleic acid can be designed to target conserved regions of an RNA sequence having homology between several genes thereby targeting several genes in a gene family (e.g., different gene isoforms, splice variants, mutant genes, etc.). In some embodiments, the regulatory nucleic acid can be designed to target a sequence that is unique to a specific RNA sequence of a single gene.

In some embodiments, the genetic element may include one or more sequences that encode regulatory nucleic acids that modulate expression of one or more genes.

In one embodiment, the gRNA described elsewhere herein are used as part of a CRISPR system for gene editing. For the purposes of gene editing, the anellovector or anelloVLP may be designed to include one or multiple guide RNA sequences corresponding to a desired target DNA sequence; see, for example, Cong et al. (2013) Science, 339:819-823; Ran et al. (2013) Nature Protocols, 8:2281-2308. At least about 16 or 17 nucleotides of gRNA sequence generally allow for Cas9-mediated DNA cleavage to occur; for Cpf1 at least about 16 nucleotides of gRNA sequence is needed to achieve detectable DNA cleavage.

### Therapeutic Effectors (e.g., Peptides or Polypeptides)

In some embodiments, the genetic element comprises a therapeutic expression sequence, e.g., a sequence that encodes a therapeutic peptide or polypeptide, e.g., an intracellular peptide or intracellular polypeptide, a secreted polypeptide, or a protein replacement therapeutic, e.g., a wild-type protein or a functional fragment or variant thereof. In some embodiments, the genetic element includes a sequence encoding a protein e.g., a therapeutic protein. Some examples of therapeutic proteins may include, but are not limited to, a hormone, a cytokine, an enzyme, an antibody (e.g., one or a plurality of polypeptides encoding at least a heavy chain or a light chain), a transcription factor, a receptor (e.g., a membrane receptor), a ligand, a membrane transporter, a secreted protein, a peptide, a carrier protein, a structural protein, a nuclease, or a component thereof.

In some embodiments, the genetic element includes a sequence encoding a peptide e.g., a therapeutic peptide. The peptides may be linear or branched. The peptide has a length from about 5 to about 500 amino acids, about 15 to about 400 amino acids, about 20 to about 325 amino acids, about 25 to about 250 amino acids, about 50 to about 200 amino acids, or any range there between.

In some embodiments, the polypeptide encoded by the therapeutic expression sequence may be a functional variant or fragment thereof of any of the above, e.g., a protein having at least 80%, 85%, 90%, 95%, 967%, 98%, 99% identity to a protein sequence which disclosed in a table herein by reference to its UniProt ID.

In some embodiments, the therapeutic expression sequence may encode an antibody or antibody fragment that binds any of the above, e.g., an antibody against a protein having at least 80%, 85%, 90%, 95%, 967%, 98%, 99% identity to a protein sequence which disclosed in a table herein by reference to its UniProt ID. The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity. An “antibody fragment” refers to a molecule that includes at least one heavy chain or light chain and binds an antigen. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

In some embodiments, an effector as described herein comprises a cytosolic polypeptide or cytosolic peptide, e.g., a wild-type protein or a functional fragment or variant thereof.

In some embodiments, the effector comprises a regulatory intracellular polypeptide, e.g., a wild-type protein or a functional fragment or variant thereof. In some embodiments, the regulatory intracellular polypeptide binds one or more molecule (e.g., protein or nucleic acid) endogenous to the target cell. In some embodiments, the regulatory intracellular polypeptide increases the level or activity of one or more molecule (e.g., protein or nucleic acid) endogenous to the target cell. In some embodiments, the regulatory intracellular polypeptide decreases the level or activity of one or more molecule (e.g., protein or nucleic acid) endogenous to the target cell.

In some embodiments, an effector as described herein comprises a secreted polypeptide effector, e.g., a wild-type protein or a functional fragment or variant thereof. Exemplary secreted therapeutics include cytokines and cytokine receptors.

Exemplary cytokines and cytokine receptors are described, e.g., in Akdis et al., “Interleukins (from IL-1 to IL-38), interferons, transforming growth factor J, and TNF-α: Receptors, functions, and roles in diseases” October 2016 Volume 138, Issue 4, Pages 984-1010.

Additional exemplary secreted therapeutics include polypeptide hormones and receptors, e.g., a wild-type protein or a functional fragment or variant thereof.

Additional exemplary secreted therapeutics include growth factors, e.g., a wild-type protein or a functional fragment or variant thereof.Exemplary growth factors and growth factor receptors are described, e.g., in Bafico et al., “Classification of Growth Factors and Their Receptors” Holland-Frei Cancer Medicine. 6th edition.

Additional exemplary secreted therapeutics include clotting-associated factors, e.g., a wild-type protein or a functional fragment or variant thereof.

In some embodiments, an effector described herein comprises a protein replacement therapeutic, e.g., a wild-type protein or a functional fragment or variant thereof. Exemplary protein replacement therapeutics are described herein.

In some embodiments, an effector described herein comprises an enzymatic effector, e.g., a wild-type protein or a functional fragment or variant thereof.

In some embodiments, an effector described herein comprises a non-enzymatic effector, e.g., a wild-type protein or a functional fragment or variant thereof.

In some embodiments, an effector described herein comprises a protein that, when mutated, causes a lysosomal storage disorder, e.g., a wild-type protein or a functional fragment or variant thereof.

In some embodiments, an effector described herein comprises a transporter protein, e.g., a wild-type protein or a functional fragment or variant thereof.

In some embodiments, a functional variant of a wild-type protein comprises a protein that has one or more activities of the wild-type protein, e.g., the functional variant catalyzes the same reaction as the corresponding wild-type protein, e.g., at a rate no less than 10%, 20%, 30%, 40%, or 50% lower than the wild-type protein. In some embodiments, the functional variant binds to the same binding partner that is bound by the wild-type protein, e.g., with a Kd of no more than 10%, 20%, 30%, 40%, or 50% higher than the Kd of the corresponding wild-type protein for the same binding partner under the same conditions. In some embodiments, the functional variant has at a polyeptpide sequence at least 70%, 75%, 80%, 85%, 90%, 95%, 96%, 97%, 98%, or 99% identical to that of the wild-type polypeptide. In some embodiments, the functional variant comprises a homolog (e.g., ortholog or paralog) of the corresponding wild-type protein. In some embodiments, the functional variant is a fusion protein. In some embodiments, the fusion comprises a first region with at least 70%, 75%, 80%, 85%, 90%, 95%, 96%, 97%, 98%, or 99% identity to the corresponding wild-type protein, and a second, heterologous region. In some embodiments, the functional variant comprises or consists of a fragment of the corresponding wild-type protein.

In some embodiments, an effector as described herein comprises a transformation factor, e.g., a wild-type protein or a fragment or variant thereof. In embodiments, the transformation factor is a protein factor that transforms fibroblasts into differentiated cells.In some embodiments, an effector as described herein comprises a protein that stimulates cellular regeneration, e.g., a wild-type protein or a fragment or variant thereof.

In some embodiments, an effector as described herein modulates STING/cGAS signaling, e.g., a wild-type protein or a fragment or variant thereof. In some embodiments, the STING modulator is a polypeptide, e.g., a viral polypeptide or a functional variant thereof. For instance, the effector may comprise a STING modulator (e.g., inhibitor) described in Maringer et al. “Message in a bottle: lessons learned from antagonism of STING signalling during RNA virus infection” Cytokine & Growth Factor Reviews Volume 25, Issue 6, December 2014, Pages 669-679. Additional STING modulators (e.g., activators) are described, e.g., in Wang et al. “STING activator c-di-GMP enhances the anti-tumor effects of peptide vaccines in melanoma-bearing mice.” Cancer Immunol Immunother. 2015 August; 64(8):1057-66. doi: 10.1007/s00262-015-1713-5. Epub 2015 May 19; Bose “cGAS/STING Pathway in Cancer: Jekyll and Hyde Story of Cancer Immune Response” Int J Mol Sci. 2017 November; 18(11): 2456; and Fu et al. “STING agonist formulated cancer vaccines can cure established tumors resistant to PD-1 blockade” Sci Transl Med. 2015 Apr. 15; 7(283): 283ra52.Some examples of peptides include, but are not limited to, fluorescent tag or marker, antigen, peptide therapeutic, synthetic or analog peptide from naturally-bioactive peptide, agonist or antagonist peptide, anti-microbial peptide, a targeting or cytotoxic peptide, a degradation or self-destruction peptide, and degradation or self-destruction peptides. Peptides useful in the invention described herein also include antigen-binding peptides, e.g., antigen binding antibody or antibody-like fragments, such as single chain antibodies, nanobodies (see, e.g., Steeland et al. 2016.

Nanobodies as therapeutics: big opportunities for small antibodies. Drug Discov Today: 21(7):1076-113). Such antigen binding peptides may bind a cytosolic antigen, a nuclear antigen, or an intra-organellar antigen.

In some embodiments, the genetic element comprises a sequence that encodes small peptides, peptidomimetics (e.g., peptoids), amino acids, and amino acid analogs. Such therapeutics generally have a molecular weight less than about 5,000 grams per mole, a molecular weight less than about 2,000 grams per mole, a molecular weight less than about 1,000 grams per mole, a molecular weight less than about 500 grams per mole, and salts, esters, and other pharmaceutically acceptable forms of such compounds. Such therapeutics may include, but are not limited to, a neurotransmitter, a hormone, a drug, a toxin, a viral or microbial particle, a synthetic molecule, and agonists or antagonists thereof.

In some embodiments, the composition, anellovector, or anelloVLP described herein includes a polypeptide linked to a ligand that is capable of targeting a specific location, tissue, or cell.

### Gene Editing Components

The genetic element of the anellovector may include one or more genes that encode a component of a gene editing system. Alternatively, an anellovector or anelloVLP as described herein may comprise a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 Oct;46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (I-III) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas endonuclease (e.g., Cas9) generates only a single-strand break; a catalytically inactive Cas endonuclease, e.g., Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence.

Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

In some embodiments, the anellovector includes a gene encoding a fusion of a dCas9 with all or a portion of one or more effector domains (e.g., a full-length wild-type effector domain, or a fragment or variant thereof, e.g., a biologically active portion thereof) to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Regulatory Sequences

In some embodiments, the genetic element comprises a regulatory sequence, e.g., a promoter or an enhancer, operably linked to the sequence encoding the effector.

In some embodiments, a promoter includes a DNA sequence that is located adjacent to a DNA sequence that encodes an expression product. A promoter may be linked operatively to the adjacent DNA sequence. A promoter typically increases an amount of product expressed from the DNA sequence as compared to an amount of the expressed product when no promoter exists. A promoter from one organism can be utilized to enhance product expression from the DNA sequence that originates from another organism. For example, a vertebrate promoter may be used for the expression of jellyfish GFP in vertebrates. In addition, one promoter element can increase an amount of products expressed for multiple DNA sequences attached in tandem. Hence, one promoter element can enhance the expression of one or more products. Multiple promoter elements are well-known to persons of ordinary skill in the art.

In one embodiment, high-level constitutive expression is desired. Examples of such promoters include, without limitation, the retroviral Rous sarcoma virus (RSV) long terminal repeat (LTR) promoter/enhancer, the cytomegalovirus (CMV) immediate early promoter/enhancer (see, e.g., Boshart et al, Cell, 41:521-530 (1985)), the SV40 promoter, the dihydrofolate reductase promoter, the cytoplasmic .beta.-actin promoter and the phosphoglycerol kinase (PGK) promoter.

In another embodiment, inducible promoters may be desired. Inducible promoters are those which are regulated by exogenously supplied compounds, either in cis or in trans, including without limitation, the zinc-inducible sheep metallothionine (MT) promoter; the dexamethasone (Dex)-inducible mouse mammary tumor virus (MMTV) promoter; the T7 polymerase promoter system (WO 98/10088); the tetracycline-repressible system (Gossen et al, Proc. Natl. Acad. Sci. USA, 89:5547-5551 (1992)); the tetracycline-inducible system (Gossen et al., Science, 268:1766-1769 (1995); see also Harvey et al., Curr. Opin. Chem. Biol., 2:512-518 (1998)); the RU486-inducible system (Wang et al., Nat. Biotech., 15:239-243 (1997) and Wang et al., Gene Ther., 4:432-441 (1997)]; and the rapamycin-inducible system (Magari et al., J. Clin. Invest., 100:2865-2872 (1997); Rivera et al., Nat. Medicine. 2:1028-1032 (1996)). Other types of inducible promoters which may be useful in this context are those which are regulated by a specific physiological state, e.g., temperature, acute phase, or in replicating cells only.

In some embodiments, a native promoter for a gene or nucleic acid sequence of interest is used. The native promoter may be used when it is desired that expression of the gene or the nucleic acid sequence should mimic the native expression. The native promoter may be used when expression of the gene or other nucleic acid sequence must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the genetic element comprises a gene operably linked to a tissue-specific promoter. For instance, if expression in skeletal muscle is desired, a promoter active in muscle may be used. These include the promoters from genes encoding skeletal α-actin, myosin light chain 2A, dystrophin, muscle creatine kinase, as well as synthetic muscle promoters with activities higher than naturally-occurring promoters. See Li et al., Nat. Biotech., 17:241-245 (1999). Examples of promoters that are tissue-specific are known for liver albumin, Miyatake et al. J. Virol., 71:5124-32 (1997); hepatitis B virus core promoter, Sandig et al., Gene Ther. 3:1002-9 (1996); alpha-fetoprotein (AFP), Arbuthnot et al., Hum. Gene Ther., 7:1503-14 (1996)], bone (osteocalcin, Stein et al., Mol. Biol. Rep., 24:185-96 (1997); bone sialoprotein, Chen et al., J. Bone Miner. Res. 11:654-64 (1996)), lymphocytes (CD2, Hansal et al., J. Immunol., 161:1063-8 (1998); immunoglobulin heavy chain; T cell receptor a chain), neuronal (neuron-specific enolase (NSE) promoter, Andersen et al. Cell. Mol. Neurobiol., 13:503-15 (1993); neurofilament light-chain gene, Piccioli et al., Proc. Natl. Acad. Sci. USA, 88:5611-5 (1991); the neuron-specific vgf gene, Piccioli et al., Neuron, 15:373-84 (1995)]; among others.

The genetic element may include an enhancer, e.g., a DNA sequence that is located adjacent to the DNA sequence that encodes a gene. Enhancer elements are typically located upstream of a promoter element or can be located downstream of or within a coding DNA sequence (e.g., a DNA sequence transcribed or translated into a product or products). Hence, an enhancer element can be located 100 base pairs, 200 base pairs, or 300 or more base pairs upstream or downstream of a DNA sequence that encodes the product. Enhancer elements can increase an amount of recombinant product expressed from a DNA sequence above increased expression afforded by a promoter element. Multiple enhancer elements are readily available to persons of ordinary skill in the art.

In some embodiments, the genetic element comprises one or more inverted terminal repeats (ITR) flanking the sequences encoding the expression products described herein. In some embodiments, the genetic element comprises one or more long terminal repeats (LTR) flanking the sequence encoding the expression products described herein. Examples of promoter sequences that may be used, include, but are not limited to, the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, and a Rous sarcoma virus promoter.

### Replication Proteins

In some embodiments, the genetic element of the anellovector, e.g., synthetic anellovector, may include sequences that encode one or more replication proteins. In some embodiments, the anellovector may replicate by a rolling-circle replication method, e.g., synthesis of the leading strand and the lagging strand is uncoupled. In such embodiments, the anellovector comprises three elements additional elements: i) a gene encoding an initiator protein, ii) a double strand origin, and iii) a single strand origin. A rolling circle replication (RCR) protein complex comprising replication proteins binds to the leading strand and destabilizes the replication origin. The RCR complex cleaves the genome to generate a free 3′OH extremity. Cellular DNA polymerase initiates viral DNA replication from the free 3′OH extremity. After the genome has been replicated, the RCR complex closes the loop covalently. This leads to the release of a positive circular single-stranded parental DNA molecule and a circular double-stranded DNA molecule composed of the negative parental strand and the newly synthesized positive strand. The single-stranded DNA molecule can be either encapsidated or involved in a second round of replication. See for example, Virology Journal 2009, 6:60 doi:10.1186/1743-422X-6-60.

The genetic element may comprise a sequence encoding a polymerase, e.g., RNA polymerase or a DNA polymerase.

### Other Sequences

In some embodiments, the genetic element further includes a nucleic acid encoding a product (e.g., a ribozyme, a therapeutic mRNA encoding a protein, an exogenous gene).

In some embodiments, the genetic element includes one or more sequences that affect species and/or tissue and/or cell tropism (e.g. capsid protein sequences), infectivity (e.g. capsid protein sequences), immunosuppression/activation (e.g. regulatory nucleic acids), viral genome binding and/or packaging, immune evasion (non-immunogenicity and/or tolerance), pharmacokinetics, endocytosis and/or cell attachment, nuclear entry, intracellular modulation and localization, exocytosis modulation, propagation, and nucleic acid protection of the anellovector in a host or host cell.

In some embodiments, the genetic element may comprise other sequences that include DNA, RNA, or artificial nucleic acids. The other sequences may include, but are not limited to, genomic DNA, cDNA, or sequences that encode tRNA, mRNA, rRNA, miRNA, gRNA, siRNA, or other RNAi molecules. In one embodiment, the genetic element includes a sequence encoding an siRNA to target a different loci of the same gene expression product as the regulatory nucleic acid. In one embodiment, the genetic element includes a sequence encoding an siRNA to target a different gene expression product as the regulatory nucleic acid.

In some embodiments, the genetic element further comprises one or more of the following sequences: a sequence that encodes one or more miRNAs, a sequence that encodes one or more replication proteins, a sequence that encodes an exogenous gene, a sequence that encodes a therapeutic, a regulatory sequence (e.g., a promoter, enhancer), a sequence that encodes one or more regulatory sequences that targets endogenous genes (siRNA, lncRNAs, shRNA), and a sequence that encodes a therapeutic mRNA or protein.

The other sequences may have a length from about 2 to about 5000 nts, about 10 to about 100 nts, about 50 to about 150 nts, about 100 to about 200 nts, about 150 to about 250 nts, about 200 to about 300 nts, about 250 to about 350 nts, about 300 to about 500 nts, about 10 to about 1000 nts, about 50 to about 1000 nts, about 100 to about 1000 nts, about 1000 to about 2000 nts, about 2000 to about 3000 nts, about 3000 to about 4000 nts, about 4000 to about 5000 nts, or any range therebetween.

### Encoded Genes

For example, the genetic element may include a gene associated with a signaling biochemical pathway, e.g., a signaling biochemical pathway-associated gene or polynucleotide. Examples include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease.

Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.). Examples of disease-associated genes and polynucleotides are listed in Tables A and B of U.S. Pat. No. 8,697,359, which are herein incorporated by reference in their entirety. Disease specific information is available from McKusick-Nathans Institute of Genetic Medicine, Johns Hopkins University (Baltimore, Md.) and National Center for Biotechnology Information, National Library of Medicine (Bethesda, Md.). Examples of signaling biochemical pathway-associated genes and polynucleotides are listed in Tables A-C of U.S. Pat. No. 8,697,359, which are herein incorporated by reference in their entirety.

Moreover, the genetic elements can encode targeting moieties, as described elsewhere herein. This can be achieved, e.g., by inserting a polynucleotide encoding a sugar, a glycolipid, or a protein, such as an antibody. Those skilled in the art know additional methods for generating targeting moieties.

### Viral Sequence

In some embodiments, the genetic element comprises at least one viral sequence. In some embodiments, the sequence has homology or identity to one or more sequence from a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the sequence has homology or identity to one or more sequence from a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the sequence has homology or identity to one or more sequence from an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus.

In some embodiments, the genetic element may comprise one or more sequences from a non-pathogenic virus, e.g., a symbiotic virus, e.g., a commensal virus, e.g., a native virus, e.g., an Anellovirus. Recent changes in nomenclature have classified the three Anelloviruses able to infect human cells into Alphatorquevirus (TT), Betatorquevirus (TTM), and Gammatorquevirus (TTMD) Genera of the Anelloviridae family of viruses. To date Anelloviruses have not been linked to any human disease. In some embodiments, the genetic element may comprise a sequence with homology or identity to a Torque Teno Virus (TT), a non-enveloped, single-stranded DNA virus with a circular, negative-sense genome. In some embodiments, the genetic element may comprise a sequence with homology or identity to a SEN virus, a Sentinel virus, a TTV-like mini virus, and a TT virus. Different types of TT viruses have been described including TT virus genotype 6, TT virus group, TTV-like virus DXL1, and TTV-like virus DXL2. In some embodiments, the genetic element may comprise a sequence with homology or identity to a smaller virus, Torque Teno-like Mini Virus (TTM), or a third virus with a genomic size in between that of TTV and TTMV, named Torque Teno-like Midi Virus (TTMD). In some embodiments, the genetic element may comprise one or more sequences or a fragment of a sequence from a non-pathogenic virus having at least about 60%, 70% 80%, 85%, 90% 95%, 96%, 97%, 98% and 99% nucleotide sequence identity to any one of the nucleotide sequences described herein.

In some embodiments, the genetic element comprises one or more sequences with homology or identity to one or more sequences from one or more non-Anelloviruses, e.g., adenovirus, herpes virus, pox virus, vaccinia virus, SV40, papilloma virus, an RNA virus such as a retrovirus, e.g., lentivirus, a single-stranded RNA virus, e.g., hepatitis virus, or a double-stranded RNA virus e.g., rotavirus. Since, in some embodiments, recombinant retroviruses are defective, assistance may be provided order to produce infectious particles. Such assistance can be provided, e.g., by using helper cell lines that contain plasmids encoding all of the structural genes of the retrovirus under the control of regulatory sequences within the LTR. Suitable cell lines for replicating the anellovectors described herein include cell lines known in the art, e.g., A549 cells, which can be modified as described herein. Said genetic element can additionally contain a gene encoding a selectable marker so that the desired genetic elements can be identified.

In some embodiments, the genetic element includes non-silent mutations, e.g., base substitutions, deletions, or additions resulting in amino acid differences in the encoded polypeptide, so long as the sequence remains at least about 70%, 75%, 80%, 85%, 90%, 95%, 96%, 97%, 98%, or 99% identical to the polypeptide encoded by the first nucleotide sequence or otherwise is useful for practicing the present invention. In this regard, certain conservative amino acid substitutions may be made which are generally recognized not to inactivate overall protein function: such as in regard of positively charged amino acids (and vice versa), lysine, arginine and histidine; in regard of negatively charged amino acids (and vice versa), aspartic acid and glutamic acid; and in regard of certain groups of neutrally charged amino acids (and in all cases, also vice versa), (1) alanine and serine, (2) asparagine, glutamine, and histidine, (3) cysteine and serine, (4) glycine and proline, (5) isoleucine, leucine and valine, (6) methionine, leucine and isoleucine, (7) phenylalanine, methionine, leucine, and tyrosine, (8) serine and threonine, (9) tryptophan and tyrosine, (10) and for example tyrosine, tryptophan and phenylalanine. Amino acids can be classified according to physical properties and contribution to secondary and tertiary protein structure. A conservative substitution is recognized in the art as a substitution of one amino acid for another amino acid that has similar properties.

Identity of two or more nucleic acid or polypeptide sequences having the same or a specified percentage of nucleotides or amino acid residues that are the same (e.g., about 60%, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) may be measured using a BLAST or BLAST 2.0 sequence comparison algorithms with default parameters described below, or by manual alignment and visual inspection (see, e.g., NCBI web site www.ncbi.nlm.nih.gov/BLAST/or the like). Identity may also refer to, or may be applied to, the compliment of a test sequence. Identity also includes sequences that have deletions and/or additions, as well as those that have substitutions. As described herein, the algorithms account for gaps and the like. Identity may exist over a region that is at least about 10 amino acids or nucleotides in length, about 15 amino acids or nucleotides in length, about 20 amino acids or nucleotides in length, about 25 amino acids or nucleotides in length, about 30 amino acids or nucleotides in length, about 35 amino acids or nucleotides in length, about 40 amino acids or nucleotides in length, about 45 amino acids or nucleotides in length, about 50 amino acids or nucleotides in length, or more.

In some embodiments, the genetic element comprises a nucleotide sequence with at least about 75% nucleotide sequence identity, at least about 80%, 85%, 90% 95%, 96%, 97%, 98%, 99% or 100% nucleotide sequence identity to any one of the nucleotide sequences described herein, e.g., as listed in any one of Tables N1-N25. Since the genetic code is degenerate, a homologous nucleotide sequence can include any number of silent base changes, i.e., nucleotide substitutions that nonetheless encode the same amino acid.

### Gene Editing Component

The genetic element of the anellovector may include one or more genes that encode a component of a gene editing system. Exemplary gene editing systems include the clustered regulatory interspaced short palindromic repeat (CRISPR) system, zinc finger nucleases (ZFNs), and Transcription Activator-Like Effector-based Nucleases (TALEN). ZFNs, TALENs, and CRISPR-based methods are described, e.g., in Gaj et al. Trends Biotechnol. 31.7(2013):397-405; CRISPR methods of gene editing are described, e.g., in Guan et al., Application of CRISPR-Cas system in gene therapy: Pre-clinical progress in animal model. DNA Repair 2016 Oct;46:1-8. doi: 10.1016/j.dnarep.2016.07.004; Zheng et al., Precise gene deletion and replacement using the CRISPR/Cas9 system in human cells. BioTechniques, Vol. 57, No. 3, September 2014, pp. 115-124.

CRISPR systems are adaptive defense systems originally discovered in bacteria and archaea. CRISPR systems use RNA-guided nucleases termed CRISPR-associated or “Cas” endonucleases (e. g., Cas9 or Cpf1) to cleave foreign DNA. In a typical CRISPR/Cas system, an endonuclease is directed to a target nucleotide sequence (e. g., a site in the genome that is to be sequence-edited) by sequence-specific, non-coding “guide RNAs” that target single- or double-stranded DNA sequences. Three classes (1-111) of CRISPR systems have been identified. The class II CRISPR systems use a single Cas endonuclease (rather than multiple Cas proteins). One class II CRISPR system includes a type II Cas endonuclease such as Cas9, a CRISPR RNA (“crRNA”), and a trans-activating crRNA (“tracrRNA”). The crRNA contains a “guide RNA”, typically about 20-nucleotide RNA sequence that corresponds to a target DNA sequence. The crRNA also contains a region that binds to the tracrRNA to form a partially double-stranded structure which is cleaved by RNase III, resulting in a crRNA/tracrRNA hybrid. The crRNA/tracrRNA hybrid then directs the Cas9 endonuclease to recognize and cleave the target DNA sequence. The target DNA sequence must generally be adjacent to a “protospacer adjacent motif” (“PAM”) that is specific for a given Cas endonuclease; however, PAM sequences appear throughout a given genome.

In some embodiments, the anellovector includes a gene for a CRISPR endonuclease. For example, some CRISPR endonucleases identified from various prokaryotic species have unique PAM sequence requirements; examples of PAM sequences include 5′-NGG (Streptococcus pyogenes), 5′-NNAGAA (Streptococcus thermophilus CRISPRI), 5′-NGGNG (Streptococcus thermophilus CRISPR3), and 5′-NNNGATT (Neisseria meningiditis). Some endonucleases, e. g., Cas9 endonucleases, are associated with G-rich PAM sites, e. g., 5′-NGG, and perform blunt-end cleaving of the target DNA at a location 3 nucleotides upstream from (5′ from) the PAM site. Another class II CRISPR system includes the type V endonuclease Cpf1, which is smaller than Cas9; examples include AsCpf1 (from Acidaminococcus sp.) and LbCpf1 (from Lachnospiraceae sp.). Cpf1 endonucleases, are associated with T-rich PAM sites, e. g., 5′-TTN. Cpf1 can also recognize a 5′-CTA PAM motif. Cpf1 cleaves the target DNA by introducing an offset or staggered double-strand break with a 4- or 5-nucleotide 5′ overhang, for example, cleaving a target DNA with a 5-nucleotide offset or staggered cut located 18 nucleotides downstream from (3′ from) from the PAM site on the coding strand and 23 nucleotides downstream from the PAM site on the complimentary strand; the 5-nucleotide overhang that results from such offset cleavage allows more precise genome editing by DNA insertion by homologous recombination than by insertion at blunt-end cleaved DNA. See, e. g., Zetsche et al. (2015) Cell, 163:759-771.

A variety of CRISPR associated (Cas) genes may be included in the anellovector. Specific examples of genes are those that encode Cas proteins from class II systems including Cas1, Cas2, Cas3, Cas4, Cas5, Cas6, Cas7, Cas8, Cas9, Cas10, Cpf1, C2C1, or C2C3. In some embodiments, the anellovector includes a gene encoding a Cas protein, e.g., a Cas9 protein, may be from any of a variety of prokaryotic species. In some embodiments, the anellovector includes a gene encoding a particular Cas protein, e.g., a particular Cas9 protein, is selected to recognize a particular protospacer-adjacent motif (PAM) sequence. In some embodiments, the anellovector includes nucleic acids encoding two or more different Cas proteins, or two or more Cas proteins, may be introduced into a cell, zygote, embryo, or animal, e.g., to allow for recognition and modification of sites comprising the same, similar or different PAM motifs. In some embodiments, the anellovector includes a gene encoding a modified Cas protein with a deactivated nuclease, e.g., nuclease-deficient Cas9.

Whereas wild-type Cas9 protein generates double-strand breaks (DSBs) at specific DNA sequences targeted by a gRNA, a number of CRISPR endonucleases having modified functionalities are known, for example: a “nickase” version of Cas9 generates only a single-strand break; a catalytically inactive Cas9 (“dCas9”) does not cut the target DNA. A gene encoding a dCas9 can be fused with a gene encoding an effector domain to repress (CRISPRi) or activate (CRISPRa) expression of a target gene. For example, the gene may encode a Cas9 fusion with a transcriptional silencer (e.g., a KRAB domain) or a transcriptional activator (e.g., a dCas9-VP64 fusion). A gene encoding a catalytically inactive Cas9 (dCas9) fused to FokI nuclease (“dCas9-FokI”) can be included to generate DSBs at target sequences homologous to two gRNAs. See, e. g., the numerous CRISPR/Cas9 plasmids disclosed in and publicly available from the Addgene repository (Addgene, 75 Sidney St., Suite 550A, Cambridge, MA 02139; addgene.org/crispr/). A “double nickase” Cas9 that introduces two separate double-strand breaks, each directed by a separate guide RNA, is described as achieving more accurate genome editing by Ran et al. (2013) Cell, 154:1380-1389.

CRISPR technology for editing the genes of eukaryotes is disclosed in US Patent Application Publications 2016/0138008A1 and US2015/0344912A1, and in U.S. Pat. Nos. 8,697,359, 8,771,945, 8,945,839, 8,999,641, 8,993,233, 8,895,308, 8,865,406, 8,889,418, 8,871,445, 8,889,356, 8,932,814, 8,795,965, and 8,906,616. Cpf1 endonuclease and corresponding guide RNAs and PAM sites are disclosed in US Patent Application Publication 2016/0208243 A1.

In some embodiments, the anellovector comprises a gene encoding a polypeptide described herein, e.g., a targeted nuclease, e.g., a Cas9, e.g., a wild type Cas9, a nickase Cas9 (e.g., Cas9 D10A), a dead Cas9 (dCas9), eSpCas9, Cpf1, C2C1, or C2C3, and a gRNA. The choice of genes encoding the nuclease and gRNA(s) is determined by whether the targeted mutation is a deletion, substitution, or addition of nucleotides, e.g., a deletion, substitution, or addition of nucleotides to a targeted sequence.

Genes that encode a catalytically inactive endonuclease e.g., a dead Cas9 (dCas9, e.g., D10A; H840A) tethered with all or a portion of (e.g., biologically active portion of) an (one or more) effector domain (e.g., VP64) create chimeric proteins that can modulate activity and/or expression of one or more target nucleic acids sequences.

As used herein, a “biologically active portion of an effector domain” is a portion that maintains the function (e.g. completely, partially, or minimally) of an effector domain (e.g., a “minimal” or “core” domain). In some embodiments, the anellovector includes a gene encoding a fusion of a dCas9 with all or a portion of one or more effector domains to create a chimeric protein useful in the methods described herein. Accordingly, in some embodiments, the anellovector includes a gene encoding a dCas9-methylase fusion. In other some embodiments, the anellovector includes a gene encoding a dCas9-enzyme fusion with a site-specific gRNA to target an endogenous gene.

In other aspects, the anellovector includes a gene encoding 1, 2, 3, 4, 5, 6, 7, 8, 9, 10, 11, 12, 13, 14, 15, 16, 17, 18, 19, 20, or more effector domains (all or a biologically active portion) fused with dCas9.

### Proteinaceous Exterior

In some embodiments, the anellovector, e.g., synthetic anellovector, comprises a proteinaceous exterior that encloses the genetic element. In some embodiments, the anelloVLP, e.g., synthetic anelloVLP, comprises a proteinaceous exterior and an effector (e.g., an exogenous effector). The proteinaceous exterior can comprise a substantially non-pathogenic exterior protein that fails to elicit an unwanted immune response in a mammal. The proteinaceous exterior of the anellovectors or anelloVLPs typically comprises a substantially non-pathogenic protein that may self-assemble into an icosahedral formation that makes up the proteinaceous exterior.

In some embodiments, the proteinaceous exterior protein is encoded by a sequence of the genetic element of the anellovector (e.g., is in cis with the genetic element). In other embodiments, the proteinaceous exterior protein is encoded by a nucleic acid separate from the genetic element of the anellovector (e.g., is in trans with the genetic element).

In some embodiments, the protein, e.g., substantially non-pathogenic protein and/or proteinaceous exterior protein, comprises one or more glycosylated amino acids, e.g., 2, 3, 4, 5, 6, 7, 8, 9, 10, or more.

In some embodiments, the protein, e.g., substantially non-pathogenic protein and/or proteinaceous exterior protein comprises at least one hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges.

In some embodiments, the protein is a capsid protein, e.g., has a sequence having at least about 60%, 65%, 70%, 75%, 80%, 85%, 90% 95%, 96%, 97%, 98%, 99%, or 100% sequence identity to a protein encoded by any one of the nucleotide sequences encoding a capsid protein described herein, e.g., an Anellovirus ORF1 sequence or a capsid protein sequence as listed in any one of Tables A1-A25. In some embodiments, the protein or a functional fragment of a capsid protein is encoded by a nucleotide sequence having at least about 60%, 70% 80%, 85%, 90% 95%, 96%, 97%, 98%, 99%, or 100% sequence identity to any one of the nucleotide sequences described herein, e.g., an Anellovirus capsid sequence or a capsid protein sequence as listed in any one of Tables A1-A25. In some embodiments, the protein comprises a capsid protein or a functional fragment of a capsid protein that is encoded by a capsid nucleotide sequence or a sequence having at least about 60%, 65%, 70%, 75%, 80%, 85%, 90% 95%, 96%, 97%, 98%, 99%, or 100% nucleotide sequence identity to any one of the nucleotide sequences described herein, e.g., an Anellovirus capsid sequence or a capsid protein sequence as listed in any one of Tables N1-N25.

In some embodiments, the anellovector comprises a nucleotide sequence encoding a capsid protein or a functional fragment of a capsid protein or a sequence having at least about 60%, 70% 80%, 85%, 90% 95%, 96%, 97%, 98%, 99%, or 100% sequence identity to any one of the amino acid sequences described herein, e.g., an Anellovirus capsid sequence or a capsid protein sequence in any one of Tables A1-A25. In some embodiments, the anellovector comprises a nucleotide sequence encoding a capsid protein or a functional fragment of a capsid protein or a sequence having at least about 60%, 65%, 70%, 75%, 80%, 85%, 90% 95%, 96%, 97%, 98%, 99%, or 100% sequence identity to any one of the amino acid sequences described herein, e.g., an Anellovirus capsid sequence or a capsid protein sequence in any one of Tables A1-A25.

In some embodiments, the anellovector comprises a nucleotide sequence encoding an amino acid sequence having about position 1 to about position 150 (e.g., or any subset of amino acids within each range, e.g., about position 20 to about position 35, about position 25 to about position 30, about position

26 to about 30), about position 150 to about position 390 (e.g., or any subset of amino acids within each range, e.g., about position 200 to about position 380, about position 205 to about position 375, about position 205 to about 371), about 390 to about position 525, about position 525 to about position 850 (e.g., or any subset of amino acids within each range, e.g., about position 530 to about position 840, about position 545 to about position 830, about position 550 to about 820), about 850 to about position 950 (e.g., or any subset of amino acids within each range, e.g., about position 860 to about position 940, about position 870 to about position 930, about position 880 to about 923) of the amino acid sequences described herein, an Anellovirus amino acid sequence, e.g., as listed in any one of Tables A1-A25, or a functional fragment thereof. In some embodiments, the protein comprises an amino acid sequence or a functional fragment thereof or a sequence having at least about 60%, 65%, 70%, 75%, 80%, 85%, 90% 95%, 96%, 97%, 98%, 99%, or 100% sequence identity to about position 1 to about position 150 (e.g., or any subset of amino acids within each range as described herein), about position 150 to about position

390, about position 390 to about position 525, about position 525 to about position 850, about position 850 to about position 950 of the amino acid sequences described herein, an Anellovirus amino acid sequence, e.g., as listed in any one of Tables A1-A25.

In some embodiments, the protein comprises an amino acid sequence or a functional fragment thereof or a sequence having at least about 60%, 65%, 70%, 75%, 80%, 85%, 90% 95%, 96%, 97%, 98%, 99%, or 100% sequence identity to any one of the amino acid sequences or ranges of amino acids described herein, an Anellovirus amino acid sequence, e.g., as listed in any one of Tables A1-A25. In some embodiments, the ranges of amino acids with less sequence identity may provide one or more of the properties described herein and differences in cell/tissue/species specificity (e.g. tropism).

In some embodiments, the anellovector or anelloVLP lacks lipids in the proteinaceous exterior. In some embodiments, the anellovector or anelloVLP lacks a lipid bilayer, e.g., a viral envelope. In some embodiments, the interior of the anellovector or anelloVLP is entirely covered (e.g., 100% coverage) by a proteinaceous exterior. In some embodiments, the interior of the anellovector or anelloVLP is less than 100% covered by the proteinaceous exterior, e.g., 95%, 90%, 85%, 80%, 70%, 60%, 50% or less coverage. In some embodiments, the proteinaceous exterior comprises gaps or discontinuities, e.g., permitting permeability to water, ions, peptides, or small molecules, e.g., so long as the genetic element is retained in the anellovector.

In some embodiments, the proteinaceous exterior comprises one or more proteins or polypeptides that specifically recognize and/or bind a host cell, e.g., a complementary protein or polypeptide, to mediate entry of the genetic element into the host cell.

In some embodiments, the proteinaceous exterior comprises one or more of the following: one or more glycosylated proteins, a hydrophilic DNA-binding region, an arginine-rich region, a threonine-rich region, a glutamine-rich region, a N-terminal polyarginine sequence, a variable region, a C-terminal polyglutamine/glutamate sequence, and one or more disulfide bridges. For example, the proteinaceous exterior comprises a protein encoded by an Anellovirus ORF1 described herein.

In some embodiments, the proteinaceous exterior comprises one or more of the following characteristics: an icosahedral symmetry, recognizes and/or binds a molecule that interacts with one or more host cell molecules to mediate entry into the host cell, lacks lipid molecules, lacks carbohydrates, is pH and temperature stable, is detergent resistant, and is substantially non-immunogenic or non-pathogenic in a host.

### Surface Moieties

An anellovector or anelloVLP as described herein may, in some instances, include one or more moieties attached to its surface (e.g., a surface moiety that can act as an effector and/or a targeting agent). In some instances, an anellovector or anelloVLP comprises more than one distinct surface moiety (e.g., a first surface moiety having an effector function as described herein and a second surface moiety that targets the anellovector or anelloVLP to a cell or tissue of interest). In some instances, the surface moiety is covalently attached to the surface of the anellovector or anelloVLP. For example, the surface moiety may be covalently attached to the proteinaceous exterior or a component thereof (e.g., covalently attached to an ORF1 molecule of the proteinaceous exterior). In certain embodiments, the surface moiety is fused to an ORF1 molecule. In some instances, the surface moiety is noncovalently attached to the surface of the anellovector or anelloVLP. For example, the surface moiety may be noncovalently bound to the proteinaceous exterior or a component thereof (e.g., noncovalently bound to an ORF1 molecule of the proteinaceous exterior). In certain embodiments, the surface moiety comprises a region that specifically binds to a cognate moiety on or attached to the ORF1 molecule. In an embodiment, the ORF1 molecule comprises a binding moiety (e.g., an antibody molecule) that specifically recognizes an epitope on the region on the surface moiety. In an embodiment, the surface moiety comprises a binding moiety (e.g., an antibody molecule) that specifically recognizes an epitope on the ORF1 molecule. In an embodiment, the surface moiety comprises a streptavidin moiety that binds to a biotin moiety on the surface of the anellovector or anelloVLP (e.g., a biotin moiety attached to an ORF1 molecule of the proteinaceous exterior of the anellovector or anelloVLP). In an embodiment, the surface moiety comprises a biotin moiety that binds to a streptavidin moiety on the surface of the anellovector or anelloVLP (e.g., a streptavidin moiety attached to an ORF1 molecule of the proteinaceous exterior of the anellovector or anelloVLP).

In embodiments, all copies of an ORF1 molecule in the proteinaceous exterior of an anellovector or anelloVLP are attached to copies of the surface moiety. In embodiments, some copies of the ORF1 molecule in the proteinaceous exterior of an anellovector or anelloVLP are attached to copies of the surface moiety and some copies of the ORF1 molecule in the proteinaceous exterior of an anellovector or anelloVLP are not attached to copies of the surface moiety. In embodiments, some copies of the ORF1 molecule in the proteinaceous exterior of an anellovector or anelloVLP are attached to copies of a first surface moiety and some copies of the ORF1 molecule in the proteinaceous exterior of an anellovector or anelloVLP are attached to copies of a second surface moiety. In embodiments, some copies of the ORF1 molecule in the proteinaceous exterior of an anellovector or anelloVLP are attached to copies of a third surface moiety.

In some embodiments, all copies of an ORF1 molecule in the proteinaceous exterior of an anellovector or anelloVLP are attached at the same position (e.g., a lysine residue) of the ORF1 molecule to a copy of a surface moiety. In some embodiments, a first copy of an ORF1 molecule in the proteinaceous exterior of an anellovector or anelloVLP is attached at a first position (e.g., a first lysine residue) of the ORF1 molecule to a first copy of a surface moiety, and a second copy of an ORF1 molecule in the proteinaceous exterior of an anellovector or anelloVLP is attached at a second position (e.g., a second lysine residue) of the ORF1 molecule to a second of a surface moiety. In some embodiments, the proteinaceous exterior further comprises one or more copies of an ORF1 molecule having a surface moiety attached to a one or more additional positions (e.g., one or more additional lysine residues). In certain embodiments, the first lysine residue, the second lysine residue, and/or the one or more additional lysine residues are positioned on the surface of the proteinaceous exterior. In some embodiments, the surface moiety is attached to the ORF1 molecule via click chemistry or genetic grafting, e.g., as described herein.

In some instances, the surface moiety comprises an effector function (e.g., as described herein). For example, the surface moiety may modulate a biological activity, e.g., of a target cell or organ. In some instances, the surface moiety induces modulation of the biological activity via binding to a cognate moiety on a target cell. For example, the surface moiety may comprise a ligand that binds to a receptor on the surface of the target cell, e.g., wherein binding of the surface moiety to the receptor initiates a downstream signaling cascade of interest. In some instances, the effector activity comprises increasing or decreasing enzymatic activity, gene expression, cell signaling, and/or cellular or organ function within a target cell or organ. Effector activities may also include binding regulatory proteins to modulate activity of the regulator, such as transcription or translation. Effector activities also may include activator or inhibitor functions.

In some instances, the surface moiety can target the anellovector or anelloVLP to a target cell. For example, the surface moiety may specifically bind to a cognate moiety on the surface of the target cell. The cognate moiety on the surface of the target cell may be, for example, a molecule specifically expressed or preferentially expressed by the target cell. The cognate moiety may be, for example, a polypeptide, lipid, sugar, or small molecule. In certain embodiments, the cognate moiety is a transmembrane protein (e.g., comprising an extracellular domain that binds to the surface moiety of the anellovector or the anelloVLP). In certain embodiments, the cognate moiety is tethered to the surface of the cell (e.g., via a GPI anchor). In some instances, the surface moiety provides a tropism (e.g., to a target tissue or target cell type) for the anellovector or anelloVLP.

In some instances, the surface moiety comprises an effector function and a targeting function, e.g., as described herein. In some instances, the surface moiety comprises a domain having an effector function as described herein. In some instances, the surface moiety comprises a domain having a targeting function as described herein.

In some instances, the surface moiety binds specifically to one cognate moiety. In some embodiments, the surface moiety binds specifically to more than one cognate moiety. In some embodiments, the surface moiety comprises a plurality of binding regions, for example, each of which specifically binds to a different cognate moiety. For example, the surface moiety may be bispecific or trispecific. In some embodiments, the surface moiety comprises a plurality of binding regions, for example, each of which binds to the same cognate moiety or copies thereof (e.g., at different epitopes of the cognate moiety or the same epitope of the cognate moiety). In certain embodiments, the surface moiety having multiple binding regions that specifically bind to the same cognate moiety results in greater avidity for the target moiety.

### Click Chemistry

In an aspect, the disclosure provides an ORF1 molecule comprising: (i) the amino acid sequence of an Anellovirus ORF1 protein, or an amino acid sequence having at least 75%, 80%, 85%, 90%, 95%, 96%, 97%, 98%, or 99% sequence identity thereto; and (ii) a click handle (e.g., an NHS click handle or a maleimide click handle, e.g., as described herein). In certain embodiments, the click handle is covalently attached to the ORF1 molecule. In certain embodiments, the click handle is noncovalently attached to the ORF1 molecule. In certain embodiments, the click handle is used to attach the ORF1 molecule to a surface moiety, e.g., via a click reaction, e.g., as described herein.

A “click handle,” as that term is used herein, refers to a chemical moiety that is capable of reacting with a second click handle in a click reaction. In some embodiments, a click handle comprises an NHS moiety and/or a maleimide moiety. In certain embodiments, a click handle comprises a DBCO moiety. In certain embodiments, a click handle comprises an azide moiety. In some embodiments, a click handle is attached to a polypeptide (e.g., an ORF1 molecule). In other embodiments, a click handle comprises a reactive group capable of forming a covalent bond with a polypeptide (e.g., an ORF1 molecule). A “click reaction”, as that term is used herein, refers to a range of reactions used to covalently link a first and a second moiety, for convenient production of linked products. It typically has one or more of the following characteristics: it is fast, is specific, is high-yield, is efficient, is spontaneous, does not significantly alter biocompatibility of the linked entities, has a high reaction rate, produces a stable product, favors production of a single reaction product, has high atom economy, is chemoselective, is modular, is stereoselective, is insensitive to oxygen, is insensitive to water, is high purity, generates only inoffensive or relatively non-toxic byproducts that can be removed by nonchromatographic methods (e.g., crystallization or distillation), needs no solvent or can be performed in a solvent that is benign or physiologically compatible, e.g., water, stable under physiological conditions. Examples include an alkyne/azide reaction, a diene/dienophile reaction, or a thiol/alkene reaction. Other reactions can be used.

In embodiments, a click reaction has a second order forward rate constant of 10-200 M-1s-1, 1-20 M-1s-1, or at least 1, 2, 3, 5, 10, 20, 50, 60, 100, 200, 500, 1E3, 2E3, 5E3, 1E4, 2E4, 5E4, 1E5, 2E5, 5E5, or 1E6 M-1s-1, e.g., at 20° C. in PBS. In some embodiments, a click reaction has at least 75%, 80%, 85%, 90%, 95%, 96%, 97%, 98%, or 99% yield, e.g., for a reaction time of 1 hour at 20° C. in PBS.

In some embodiments, a surface moiety is attached to a polypeptide by conjugation of a lysine residue on the surface of the polypeptide to an NHS click handle on the surface moiety (e.g., as described in Example 2 or 3 herein). In some embodiments, a surface moiety is attached to a polypeptide by conjugation of a lysine residue on the surface of the surface moiety to an NHS click handle on the polypeptide (e.g., as described in Example 2 or 3 herein).

In some embodiments, a surface moiety is attached to a polypeptide by conjugation of a cysteine residue on the surface of the polypeptide to a maleimide click handle on the surface moiety (e.g., as described in Example 4 herein). In some embodiments, a surface moiety is attached to a polypeptide by conjugation of a cysteine residue on the surface of the surface moiety to a maleimide click handle on the polypeptide (e.g., as described in Example 4 herein).

In an aspect, the disclosure provides an ORF1 molecule comprising a surface moiety, wherein the surface moiety was attached to the ORF1 molecule via a click reaction.

In an aspect, the disclosure provides a particle (e.g., an anellovector or anelloVLP) comprising: (i) a proteinaceous exterior comprising an ORF1 molecule; and (ii) a click handle (e.g., an NHS click handle and/or a maleimide click handle, e.g., as described herein). In certain embodiments, the click handle is covalently attached to the ORF1 molecule. In some embodiments, the particle is an anellovector comprising a genetic element enclosed in the proteinaceous exterior. In some embodiments, the particle is an anelloVLP comprising an effector (e.g., an exogenous effector), e.g., enclosed in the proteinaceous exterior.

### Mutations of Surface Lysines

In an aspect, the disclosure provides an ORF1 molecule comprising the amino acid sequence of an Anellovirus ORF1 protein (or an amino acid sequence having at least 75%, 80%, 85%, 90%, 95%, 96%, 97%, 98%, or 99% sequence identity thereto), wherein at least 30%, 40%, 50%, 60%, 70%, 75%, 80%, 85%, 90%, 95%, 96%, 97%, 98%, 99%, or 100% of the lysine residues in the amino acid sequence of the Anellovirus ORF1 protein has been mutated (e.g., substituted with another amino acid, e.g., threonine, alanine, serine, asparagine, or glutamine). In some embodiments, all but one lysine residue of the Anellovirus ORF1 protein that are exposed from the surface of a proteinaceous exterior comprising the Anellovirus ORF1 protein are mutated (e.g., substituted with another amino acid, e.g., serine or alanine). In some embodiments, all lysine residues of the Anellovirus ORF1 protein that are exposed from the surface of a proteinaceous exterior comprising the Anellovirus ORF1 protein are mutated (e.g., substituted with another amino acid, e.g., serine or alanine). In some embodiments, all but one of the lysine residues of the Anellovirus ORF1 protein are mutated (e.g., substituted with another amino acid, e.g., serine or alanine). In certain embodiments, the one lysine residue not mutated is exposed on the surface of a proteinaceous exterior comprising the Anellovirus ORF1 protein.

In some embodiments, all lysine residues of the Anellovirus ORF1 protein are mutated (e.g., substituted with another amino acid, e.g., serine or alanine). In some embodiments, the ORF1 molecule further comprises a lysine residue not found in the amino acid sequence of the Anellovirus ORF1 protein (e.g., a lysine residue inserted or substituted into the Anellovirus ORF1 protein sequence, or a lysine residue attached to the N-terminal or C-terminal end of the Anellovirus ORF1 protein sequence).

Such mutated ORF1 molecules may be useful, for example, for controlling covalent attachment of a surface moiety to a lysine residue in the proteinaceous exterior (e.g., via a NHS click reaction as described herein, e.g., in Example 2 or 3).

### Mutations of Surface Cysteines

In an aspect, the disclosure provides an ORF1 molecule comprising the amino acid sequence of an Anellovirus ORF1 protein (or an amino acid sequence having at least 75%, 80%, 85%, 90%, 95%, 96%, 97%, 98%, or 99% sequence identity thereto), wherein at least 30%, 40%, 50%, 60%, 70%, 75%, 80%, 85%, 90%, 95%, 96%, 97%, 98%, 99%, or 100% of the cysteine residues in the amino acid sequence of the Anellovirus ORF1 protein has been mutated (e.g., substituted with another amino acid, e.g., serine or alanine). In some embodiments, all but one cysteine residue of the Anellovirus ORF1 protein that are exposed from the surface of a proteinaceous exterior comprising the Anellovirus ORF1 protein are mutated (e.g., substituted with another amino acid, e.g., serine or alanine). In some embodiments, all cysteine residues of the Anellovirus ORF1 protein that are exposed from the surface of a proteinaceous exterior comprising the Anellovirus ORF1 protein are mutated (e.g., substituted with another amino acid, e.g., serine or alanine). In some embodiments, cysteine residues exposed on the surface of the ORF1 molecule are mutated to serine. In some embodiments, cysteine residues interior to the ORF1 molecule are mutated to alanine. In embodiments, one or more cysteine residues corresponding to positions 57, 64, 112, 131, 220, 223, 626 of Ring10 ORF1 are mutated (e.g., to serine or alanine).

In some embodiments, all but one of the cysteine residues of the Anellovirus ORF1 protein are mutated (e.g., substituted with another amino acid, e.g., serine or alanine). In certain embodiments, the one cysteine residue not mutated is exposed on the surface of a proteinaceous exterior comprising the Anellovirus ORF1 protein. In embodiments, the cysteine residue not mutated is selected from residues corresponding to positions 57, 64, 112, 131, 220, 223, 626 of Ring10 ORF1 protein.

In embodiments, the cysteine residue is located in the P2 domain of the ORF1 molecule (e.g., at a position corresponding to position 365 of Ring10 ORF1 protein, e.g., as described in Example 4). In embodiments, the cysteine residue is located in the HVR of the ORF1 molecule.

In some embodiments, all cysteine residues of the Anellovirus ORF1 protein are mutated (e.g., substituted with another amino acid, e.g., serine or alanine). In some embodiments, the ORF1 molecule further comprises a cysteine residue not found in the amino acid sequence of the Anellovirus ORF1 protein (e.g., a cysteine residue inserted or substituted into the Anellovirus ORF1 protein sequence, or a cysteine residue attached to the N-terminal or C-terminal end of the Anellovirus ORF1 protein sequence).

Such mutated ORF1 molecules may be useful, for example, for controlling covalent attachment of a surface moiety to a cysteine residue in the proteinaceous exterior (e.g., via a maleimide click reaction as described herein, e.g., in Example 4).

### Polypeptides

The surface moiety can, in some instances, comprise a polypeptide. In some embodiments, the polypeptide is a about 5-10, 10-15, 15-20, 20-25, 25-30, 30-40, 40-50, 50-100, 100-150, 150-200, 200-250, 250-300, 300-400, 400-500, 500-600, 600-700, 700-800, 800-900, 900-1000, 1000-1500, 1500-2000, 2000-2500, 2500-3000, 3000-4000, or 4000-5000 amino acids in length). In some embodiments, the surface moiety is a polypeptide fused to a protein of the anellovector or anelloVLP (e.g., an ORF1 molecule of the anellovector or anelloVLP). In certain embodiments, the peptide is linear or branched.

In some embodiments, the surface moiety comprises an antibody molecule (e.g., an antibody or an antigen-binding fragment thereof). In certain embodiments, the surface moiety comprises an Fv, Fab, Fab′, Fab′-SH, F(ab′)₂, diabody, linear antibody, single-chain antibody molecule (e.g. scFv), or a multispecific antibody formed from antibody fragments. In certain embodiments, the surface moiety is a multispecific antibody molecule (e.g., a bispecific antibody molecule or a trispecific antibody molecule). In some embodiments, the surface moiety is selected from a hormone, cytokine, enzyme, transcription factor, receptor, ligand, transporter, secreted protein, carrier protein, structural protein, or a functional fragment thereof (e.g., as described herein). In some embodiments, the surface moiety comprises a polypeptide effector (e.g., as described herein). In certain embodiments, the surface moiety comprises a therapeutic effector (e.g., as described herein). In embodiments, the surface moiety comprises a regulatory intracellular polypeptide (e.g., as described herein). In embodiments, the surface moiety comprises a secreted polypeptide effector (e.g., as described herein). In embodiments, the surface moiety comprises a viral polypeptide or peptide. In embodiments, the surface moiety comprises a SARS-CoV-2 polypeptide or peptide (e.g., a receptor binding domain, e.g., of a spike protein, e.g., as described herein), or a polypeptide having at least 75%, 80%, 85%, 90%, 95%, 96%, 97%, 98%, or 99% sequence identity thereto. In embodiments, the surface moiety comprises a polypeptide having at least 75%, 80%, 85%, 90%, 95%, 96%, 97%, 98%, 99%, or 100% amino acid sequence identity to amino acids 319-541 from SARS-CoV-2 coronavirus spike protein (e.g., according to ThermoFisher Catalog #RP-87704). In some embodiments, the surface moiety comprises a binding moiety (e.g., a biotin moiety). In some embodiments, the surface moiety comprises a fluorophore (e.g., an Alexa Fluor moiety, e.g., Alexa Fluor 647).

In some embodiments, a polypeptide surface moiety is displayed on the surface of the anellovector or anelloVLP. In some embodiments, the surface moiety is covalently attached to the surface (e.g., proteinaceous exterior) of the anellovector or anelloVLP. In certain embodiments, the surface moiety is a polypeptide fused to an ORF1 molecule. In embodiments, the surface moiety is a heterologous domain of an ORF1 molecule. In embodiments, the surface moiety replaces a region (e.g., a subdomain as described herein, e.g., an HVR) of an ORF1 protein. In embodiments, all copies of the ORF1 molecule in the proteinaceous exterior of an anellovector or anelloVLP are fused to copies of the surface moiety. In embodiments, some copies of the ORF1 molecule in the proteinaceous exterior of an anellovector or anelloVLP are fused to copies of the surface moiety and some copies of the ORF1 molecule in the proteinaceous exterior of an anellovector or anelloVLP are not fused to copies of the surface moiety. In embodiments, some copies of the ORF1 molecule in the proteinaceous exterior of an anellovector or anelloVLP are fused to copies of a first surface moiety and some copies of the ORF1 molecule in the proteinaceous exterior of an anellovector or anelloVLP are fused to copies of a second surface moiety.

In some embodiments, the surface moiety is noncovalently attached to the surface (e.g., proteinaceous exterior) of the anellovector or anelloVLP. For example, the surface moiety may comprise a binding domain that binds to a region on the surface (e.g., proteinaceous exterior) of the anellovector or anelloVLP. In an embodiment, the surface moiety comprises an antibody molecule that specifically binds to an ORF1 molecule of the proteinaceous exterior of the anellovector or anelloVLP.

### Genetic Grafting of ORF1 to a Surface Moiety

In some embodiments, an ORF1 molecule comprises the amino acid sequence of a surface moiety, e.g., as described herein. An ORF1 molecule may be fused (e.g., at the N-terminus or C-terminus) to the surface moiety. In some embodiments, a surface moiety is grafted into the sequence of the ORF1 molecule. For example, a surface moiety may be inserted within or between domains of the ORF1 molecule.

In certain embodiments, the surface moiety is grafted at or within 1-5, 5-10, 10-15, 15-20, 20-25, 25-30, 30-35, or 35-40 amino acid residues of a jelly-roll region as described herein.

In certain embodiments, the surface moiety is grafted at or within the HVR, or a portion thereof (e.g., a portion consisting of 5-10, 10-15, 15-20, 20-30, 30-40, 40-50, 50-60, 60-70, 70-80, 80-90, 90-100, 100-110, 110-120, 120-130, 130-140, or 140-150 amino acids thereof).

In certain embodiments, the surface moiety is grafted at or within 1-5, 5-10, 10-15, 15-20, 20-25, 25-30, 30-35, 35-40, 40-50, 50-60, 60-70, 70-80, 80-90, 90-100, 100-125, 125-150, 150-175, or 175-200 amino acid residues of the C-terminal end of a jelly-roll region as described herein. In certain embodiments, the surface moiety is grafted at or within 1-5, 5-10, 10-15, 15-20, 20-25, 25-30, 30-35, 35-40, 40-50, 50-60, 60-70, 70-80, 80-90, 90-100, 100-125, 125-150, 150-175, or 175-200 amino acid residues of the N-terminal end of an N22 domain or CTD as described herein.

### Exemplary ORF1 Molecules Attached to Surface Moieties

In some embodiments, a surface moiety and ORF1 fusion protein comprises an amino acid sequence as listed in Table E1 below, or an amino acid sequence having at least 75%, 80%, 85%, 90%, 95%, 96%, 97%, 98%, or 99% sequence identity thereto. In some embodiments, a surface moiety comprises an amino acid sequence as listed in Table E1 below, or an amino acid sequence having at least 75%, 80%, 85%, 90%, 95%, 96%, 97%, 98%, or 99% sequence identity thereto. In some embodiments, a surface moiety comprises a CS protein, or a functional fragment thereof (e.g., a fragment comprising the 184 C-terminal residues of a CS protein). In some embodiments, a surface moiety comprises a hepatitis B virus surface antigen. In some embodiments, a surface moiety comprises one or more (e.g., 1, 2, 3, 4, 5, or 6) NANP peptides (e.g., NANP-2 peptides). In some embodiments, a surface moiety comprises a cysteine residue (e.g., a C-terminal cysteine residue). In some embodiments, a surface moiety comprises an NHS click handle (e.g., as described herein).

In an aspect, the disclosure provides a nucleic acid molecule encoding a fusion protein comprising a surface moiety and an ORF1 molecule (e.g., as described herein).

In an aspect, the disclosure provides a polypeptide comprising a CCN5 protein and a C-terminal cysteine residue. In some embodiments, the polypeptide comprises the amino acid sequence of CCN5 CTerMCys as listed in Table E1 below, or an amino acid sequence having at least 75%, 80%, 85%, 90%, 95%, 96%, 97%, 98%, or 99% sequence identity thereto. In an aspect, the disclosure provides a polypeptide comprising an aflibercept protein and a C-terminal cysteine residue. In some embodiments, the polypeptide comprises the amino acid sequence of aflibercept as listed in Table E1 below, or an amino acid sequence having at least 75%, 80%, 85%, 90%, 95%, 96%, 97%, 98%, or 99% sequence identity thereto. In an aspect, the disclosure provides a polypeptide comprising ranibizumab and a C-terminal cysteine residue. In some embodiments, the polypeptide comprises the amino acid sequence of ranibizumab HC malE_CTermCys as listed in Table E1 below, or an amino acid sequence having at least 75%, 80%, 85%, 90%, 95%, 96%, 97%, 98%, or 99% sequence identity thereto. In some embodiments, the polypeptide comprises the amino acid sequence of scFab RaniHC-link50-LC malE_CTermCys as listed in Table E1 below, or an amino acid sequence having at least 75%, 80%, 85%, 90%, 95%, 96%, 97%, 98%, or 99% sequence identity thereto. In an aspect, the disclosure provides a polypeptide comprising bevacizumab and a C-terminal cysteine residue. In some embodiments, the polypeptide comprises the amino acid sequence of bevacizumab HC malE_CTermCys as listed in Table E1 below, or an amino acid sequence having at least 75%, 80%, 85%, 90%, 95%, 96%, 97%, 98%, or 99% sequence identity thereto.

### X-Fold Symmetry

In some embodiments, an ORF1 molecule as described herein comprises a surface moiety situated at a position such that, when the ORF1 molecule is complexed with one or more additional copies of the ORF1 molecules (each attached to another surface moiety, e.g., copies of the same surface moiety or one or more different surface moieties), the surface moieties form a multimer. In certain embodiments, a complex of two such ORF1 molecules attached to surface moieties results in the formation of a dimer of the surface moieties. In certain embodiments, a complex of three such ORF1 molecules attached to surface moieties results in the formation of a trimer of the surface moieties. In certain embodiments, a complex of five such ORF1 molecules attached to surface moieties results in the formation of a pentamer of the surface moieties.

To form a multimer of surface moieties, the surface moieties are generally attached to the ORF1 molecule at a surface-exposed portion of the ORF1 molecule. In some embodiments, the surface-exposed portion of the ORF1 molecule is part of a hypervariable region (HVR) of the ORF1 molecule, e.g., as described herein. In certain embodiments, the surface moiety is fused to or is attached at or between residues of the ORF1 molecule corresponding to positions 284-285 of a Ring10 ORF1 protein. In certain embodiments, the surface moiety is fused to or is attached at or between residues of the ORF1 molecule corresponding to positions 328-329 of a Ring10 ORF1 protein. In certain embodiments, the surface moiety is fused to or is attached at or between residues of the ORF1 molecule corresponding to positions 256-383 of a Ring10 ORF1 protein. In certain embodiments, the surface moiety is fused to or is attached at or between residues of the ORF1 molecule corresponding to positions 251-383 of a Ring10 ORF1 protein. In certain embodiments, the surface moiety is fused to or is attached at or between residues of the ORF1 molecule corresponding to positions 251-384 of a Ring10 ORF1 protein. In certain embodiments, the surface moiety is attached at the amino acid residue (e.g., a cysteine residue) corresponding to position 254, 263, 264, 265, 272, 273, 274, 276, 283, 284, 285, 287, 288, 290, 291, 308, 311, 312, 313, 314, 316, 317, 318, 319, 321, 324, 328, 329, 341, 343, 354, 358, 361, 362, 363, 364, 365, 368, 369, 371, 374, 376, 378, 380, or 381 of Ring 10 ORF1, e.g., in an ORF1 domain (e.g., within the HVR or P2 domain).

In an aspect, the disclosure provides an ORF1 molecule comprising the amino acid sequence of an ORF1 protein comprising at least one mutation (e.g., deletion, substitution, addition, insertion, or frameshift) in a surface-exposed region relative to a wild-type ORF1 protein sequence (e.g., as described herein. In some embodiments, the surface-exposed region comprises the region corresponding amino acids 251-386 of the amino acid sequence of Ring 10 ORF1 (e.g., as described herein).

In an aspect, the disclosure provides an ORF1 molecule comprising the amino acid sequence of an ORF1 protein comprising at least one mutation (e.g., deletion, substitution, addition, insertion, or frameshift) in the HVR relative to a wild-type ORF1 protein sequence (e.g., as described herein).

In some embodiments, the glutamine residue corresponding to Q287 of Ring 10 (e.g., as described herein) has been mutated (e.g., substituted). In some embodiments, the glutamine residue corresponding to Q287 of Ring 10 has been mutated to a cysteine residue. In certain embodiments, the ORF1 molecule comprises the amino acid sequence of the Ring 10 ORF1 protein, or an amino acid sequence having at least 75%, 80%, 85%, 90%, 95%, 96%, 97%, 98%, or 99% sequence identity thereto, and a Q287C mutation.

In some embodiments, the threonine residue corresponding to T365 of Ring 10 (e.g., as described herein) has been mutated (e.g., substituted). In some embodiments, the threonine residue corresponding to T365 of Ring 10 has been mutated to a cysteine residue. In certain embodiments, the ORF1 molecule comprises the amino acid sequence of the Ring 10 ORF1 protein, or an amino acid sequence having at least 75%, 80%, 85%, 90%, 95%, 96%, 97%, 98%, or 99% sequence identity thereto, and a T365C mutation.

In an aspect, the disclosure provides a polypeptide comprising an amino acid sequence as listed in Table E1 below, or an amino acid sequence having at least 75%, 80%, 85%, 90%, 95%, 96%, 97%, 98%, or 99% sequence identity thereto. In some embodiments, the polypeptide further comprises the amino acid sequence of an ORF1 protein (e.g., as described herein), or an amino acid sequence having at least 75%, 80%, 85%, 90%, 95%, 96%, 97%, 98%, or 99% sequence identity thereto.

In an aspect, the disclosure provides a nucleic acid molecule encoding a polypeptide comprising an amino acid sequence as listed in Table E1 below, or an amino acid sequence having at least 75%, 80%, 85%, 90%, 95%, 96%, 97%, 98%, or 99% sequence identity thereto. In some embodiments, the polypeptide encoded by the nucleic acid molecule further comprises the amino acid sequence of an ORF1 protein (e.g., as described herein), or an amino acid sequence having at least 75%, 80%, 85%, 90%, 95%, 96%, 97%, 98%, or 99% sequence identity thereto.In some embodiments, an anellovector as described herein comprises a proteinaceous exterior comprising an ORF1 molecule as described above. In some embodiments, an anellovector as described herein comprises a proteinaceous exterior comprising an ORF1 molecule encoded by a nucleic acid molecule as described above. In some embodiments, an anelloVLP as described herein comprises a proteinaceous exterior comprising an ORF1 molecule as described above. In some embodiments, an anelloVLP as described herein comprises a proteinaceous exterior comprising an ORF1 molecule encoded by a nucleic acid molecule as described above.

### Nucleic Acid Molecules

The surface moiety may, in some instances, comprise a nucleic acid molecule. In some embodiments, the surface moiety comprises DNA (e.g., single-stranded DNA or double-stranded DNA). In some embodiments, the surface moiety comprises RNA (e.g., single-stranded RNA or double-stranded RNA). In some embodiments, the surface moiety comprises DNA and RNA (e.g., a strand of DNA hybridized to a strand of RNA). In some embodiments, the surface moiety comprises an oligonucleotide (e.g., an oligonucleotide having a length of about 5-10, 10-15, 15-20, 20-25, 25-30, 30-35, 35-40, 40-45, 45-50, 50-60, 60-70, 70-80, 80-90, or 90-100 nucleotides). In some embodiments, the surface moiety comprises a functional nucleic acid molecule (e.g., a functional RNA). In certain embodiments, the surface moiety comprises an mRNA, siRNA, miRNA, or tRNA.

### Small Molecules

The surface moiety may, in some instances, comprise a small molecule. In some embodiments, the small molecule has a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole. In some embodiments, the small molecule is a salt, ester, or other pharmaceutically acceptable form of such compounds. Small molecules may include, but are not limited to, a neurotransmitter, a hormone, a drug, a toxin, a viral or microbial particle, a synthetic molecule, and an agonist or an antagonist. In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumor suppressors). One or a combination of molecules from the categories and examples described herein or from Orme-Johnson 2007, Methods Cell Biol. 2007; 80:813-26 (incorporated by reference in its entirety) can be used. In one embodiment, the small molecule comprises an antibiotic, anti-inflammatory drug, angiogenic or vasoactive agent, growth factor or chemotherapeutic agent.

Examples of small molecules that can be used as surface moieties as described herein include, without limitation, those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference.

### Vaccines/Antigens

In some instances, a surface moiety comprises an antigen (e.g., an antigen recognized by the immune system of a subject to be delivered the anellovector or anelloVLP). In some embodiments, a surface moiety comprises a vaccine. In some embodiments, a surface moiety comprises an epitope from a bacterium, virus, fungus, or parasite. In some embodiments, a surface moiety comprises a vaccine for a pathogen (e.g., the surface moiety comprises an antigen of the pathogen). In certain embodiments, the surface moiety comprises one or more vaccine antigens as listed in Table Vi below. In embodiments, the surface moiety comprises one vaccine antigen listed in Table Vi. In embodiments, the surface moiety comprises a plurality of distinct vaccine antigens listed in Table V1 (e.g., a plurality of distinct vaccine antigens listed in a single row of Table V1). PGP-1O8,I,M

TABLE V1
Exemplary vaccine antigens that can be included in a surface moiety of an anellovector
or anelloVLP as described herein.

Ascomycota, e.g., Eurotiomycetes, e.g., Eurotiales, e.g., Trichocomaceae, e.g., Aspergillus species antigen (e.g.,
Aspergillus versicolor AVS)
Monodnaviria, e.g., Shotokuvirae, e.g., Cossaviricota, e.g., Papovaviricetes, e.g., Sepolyvirales, e.g., Polyomaviridae,
e.g., Betapolyomavirus, e.g., BK Virus antigen
Proteobacteria, e.g., Betaproteobacteria, e.g., Burkholderiales, e.g., Alcaligenaceae, e.g., Bordetella, e.g., Bordetella
pertussis antigen (e.g., pertussis toxin, filamentous haemagglutinin, pertactin)
Spirochaetes, e.g., Spirochaetales, e.g., Spriochaetaceae, e.g., Borrelia, e.g., Borrelia burgdorferi antigen
Proteobacteria, e.g., Betaproteobacteria, e.g., Burkholderiales, e.g., Burkholderiaceae, e.g., Burkholderia, e.g.,
Burkholderia species antigen (e.g., hydratase/aldolase PhnE)
Riboviria, e.g., Orthornavirae, e.g., Kitrinoviricota, e.g. Alsuviricetes, e.g., Martellivirales, e.g., Togaviridae, e.g.,
Alphavirus, e.g., Chikungunya virus (CHIKV) antigens
Chlamydiae, e.g., Chlamydiales, e.g., Chlamydiaceae, e.g., Chlamydia, e.g., Chlamydia trachomatis antigen
Proteobacteria, e.g., Gammaproteobacteria, e.g., Vibrionales, e.g., Vibrionaceae, e.g., Vibrio, e.g., Vibrio cholerae, e.g.,
Cholera antigen (e.g., cholera toxin B subunit)
“Firmicutes”, e.g., Clostridia, e.g., Clostridiales, e.g., Peptostreptococcaceae, e.g., Clostridioides, e.g., Clostridium, e.g.,
Clostridium difficile antigen
Alveolata, e.g., Apicomplexa, e.g., Conoidasida, e.g., Eucoccidiorida, e.g., Eimeriorina, e.g., Cryoptosporidiidae, e.g.,
Cryptosporidium antigen
Duplodoaviria, e.g., Heunggongvirae, e.g., Peploviricota, e.g., Herviviricetes, e.g., Herpesvirales, e.g., Herpesviridae,
e.g., Betaberpesvirinae, e.g., Cytomegalovirus (CMV) antigen
“Actinobacteria”, e.g., Acintobacteria, e.g., Mycobacteriales, e.g., Corynebacteriaceae, e.g., Corynebacterium,
Diphtheria antigen (e.g., diphtheria toxoid)
Amoebozoa, e.g., Entamoebidae, e.g., Entamoeba, e.g., Entamoeba histolytica antigen
Riboviria, e.g., Orthornavirae, e.g., Pisuviricota, e.g., Pisoniviricetes, e.g., Picornavirales, e.g., Picornaviridae, e.g.,
Enterovirus, e.g., Enterovirus 71 antigen (e,g., VP1)
Duplodnaviria, e.g., Heunggongvirae, e.g., Peploviricota, e.g., Herviviricetes, e.g., Herpesviridae, e.g.,
Lymphocryptovirus, e.g., Epstein-Barr virus antigen
Proteobacteria, e.g., Gammaproteobacteria, e.g., Enterobacterlaes, e.g., Enterobacteriaceae, e.g., Escherichia, e.g.,
Escherichia coli antigen (e.g., bifunctional penicillin-binding protein)
Riboviria, e.g., Orthornavirae, e.g., Kitrinoviricota, e.g., Flasuviricetes, e.g., Amarillovirales, Flaviviridae genus
(Dengue Virus) antigen
Proteobacteria, e.g., Epsilonproteobacteria, e.g., Campylobacterales, e.g., Helicobacteraceae, e.g., Helicobacter, e.g.,
Helicobacter pylori antigen
Hepatitis antigen (e.g., hepatitis A (HAV), e.g., B (HBV), e.g., C (HCV), e.g., D (HDV) e.g., G (HGV) virus antigen)
Duplodnaviria, e.g., Heunggongvirae, e.g., Peploviricota, e.g., Herviviricetes, e.g., Herpesviridae, e.g.,
Alphaberpesvirinae, e.g., Simplexvirus, e.g., Herpes simplex antigen
Varidnaviria, e.g., Bamfordvirae, e.g., Preplasmiviricota, e.g., Tectiliviricetes, e.g., Rowavirales, e.g., Adenoviridae,
e.g., Aviadenovirus, e.g., Ichtadenovirus, e..g, Testadenovirus, e.g., Atadenovirus, e.g., Mastadenovirus, e.g.,
Siadenovirus Human adenovirus antigen (e.g., type 34 hexon protein)
Riboviria, e.g., Pararnavirae, e.g., Artverviricota, e.g., Revtraviricetes, e.g., Ortervirales, e.g., Retroviridae, e.g.,
Orthoretrovirinae, e.g., Lentivirus, e.g., Human immunodeficiency virus (HIV) antigen (e.g., GP-120, GP-160)
Riboviria, e.g., Orthornavirae, e.g., Negarnaviricota, e.g., Monjiviricetes, e.g., Mononegavirales, e.g., Pneumoviridae,
e.g., Metapneumovirus, e.g., Human metapneumovirus antigen
Monodoaviria, e.g., Shotokuvirae, e.g., Cossaviricota, e.g., Papovaviricetes, e.g., Zurhausenvirales, e.g.,
Papillomaviridae, e.g, Alphapapillomavirus, e.g., Betapapillomavirus, e.g., Chipapillomavirus, e.g., Deltapapillovirus,
e.g., Dyochipapillomavirus, e.g., Dyodeltapapillomavirus, e.g., Dyoepsilonpapillomavirus, e.g., Dyoetapapillomavirus,
e.g., Dyoiotapapillomavirus, e.g., Dyokappapapillomavirus, e.g., Dyolambdapapillomavirus, e.g.,
Dyomupapillomavirus, e.g., Dyonupapillomavirus, e.g., Dyoomegapapillomavirus, e.g., Dyoomikronpapillomavirus,
e.g., Dyophipapillomavirus, e.g., Dyopipapillomavirus, e.g., Dyopsipapillomavirus, e.g., Dyorhopapillomavirus, e.g.,
Dyosigmapapillomavirus, e.g., Dyotaupapillomavirus, e.g., Dyothetapapillomavirus, e.g., Dyoupsilonpapillomavirus,
e.g., Dyoxipapillomavirus, e.g., Dyozetapapillomavirus e.g., Gammapapillomavirus, e.g., Iotapapillomavirus, e.g.,
Kappapapillomavirus, e.g., Lambdapapillomavirus, e.g., Mupapillomavirus, e.g., Nupapillomavirus, e.g.,
Omegapapillomavirus, e.., Omikronpapillomavirus, e.g., Phipapillomavirus, e.g., Pipapillomavirus, e.g.,
Psipapillomavirus, e.g., Rhopapillomavirus, e.g., Sigmapapillomavirus, e.g., Taupapillomavirus, e.g.,
Tbetapapillomavirus, e.g., Treisdeltapapillomavirus, e.g., Treisepsiolonpapillomavirus, e.g., Treisetapapillomavirus,
e.g., Treisiotapapillomavirus, e.g., Trieskappapapillomavirus, e.g., Treisthetapapillomavirus, e.g.,
Treiszetapapillomavirus, e.g., Upsilonpapillomavirus, e.g., Xipapillomavirus, e.g., Zetapapillomavirus, e.g.,
Alefpapillomavirus, e.g., Human papilloma virus (HPV) antigen (e.g., L1 major capsid protein)
Riboviria, e.g., Orthornavirae, e.g., Negarnaviricota, e.g., Insthoviricetes, e.g., Articulavirales, e.g., Orthomyxoviridae,
e.g., Alphainfluenzavirus, e.g., Betainfluenzavirus, e.g., Gammainfluenzavirus, e.g., Deltainfluenzavirus, e.g., Influenza
virus antigen (e.g., HA)
Monodnaviria, e.g., Shotokuvirae, e.g., Cossaviricota, e.g., Papovaviricetes, e.g., Sepolyvirales, e.g., Polyomaviridae,
e.g., Betapolyomavirus, e.g., JC Virus/JC polyomavirus antigen
Proteobacteria, e.g., gammaproteobacteria, e.g., Enterobacterales, e.g., Klebsiella, e.g., Klebsiella pneumoniae antigen
Riboviria, e.g., Orthornavirae, e.g., Negarnaviricota, e.g., Ellioviricetes, e.g., Bunyavirales, e.g., Arenaviridae, e.g.,
Mammarenavirus, e.g., Lassa virus antigen
Euglenozoa, e.g., Kinetoplastea, e.g., Trypanosomatida, e.g., Leishmania promastigotes antigen
Spriochaetes, e.g., Spirochaetales, e.g., Leptospiraceae, e.g., Leptospira, e.g., Leptospira interrogans antigen
Proteobacteria, e.g., Betaproteobacteria, e.g., Neisseriales, e.g., Neisseriaceae, e.g., Neisseria, e.g., Meningococcal
antigen (e.g., capsular polysaccharides of serogroup A, B, C, Y and W-135)
Actinobacteria, e.g., Corynebacteriales, e.g., Mycobacteriaceae, e.g., Mycobacterium, e.g., Mycobacterium tuberculosis
antigen
Proteobacteria, e.g., etaproteobacteria, e.g., Neisseriales, e.g., Neisseriaceae, e.g., Neisseria, e.g., N.
meningitidis antigen (e.g., type Y polysaccharide [MenY], type W polysaccharide [MenW])
Proteobacteria, e.g., etaproteobacteria, e.g., Neisseriales, e.g., Neisseriaceae, e.g., Neisseria, e.g., Neisseriea gonorrhea
antigen
Apicomplexa, e.g., Aconoidasida, e.g., Haemospororida, e.g., Plasmodiidae, e.g., Plasmodium, e.g., Plasmodium
species antigen (e.g., HRPII, pLDH, pAldo)
“Firmicutes”, e.g., Bacilli, e.g., Lactobacillies, e.g., Streptococcaceae, e.g., Streptococcus, e.g., Pneumococcal antigen
(e.g., Streptococcus pneumoniae type 3 capsular polysaccharide)
Proteobacteria, e.g., Gammaproteobacteria, e.g., Pseudomonadales, e.g., Pseudomonadaceae, e.g., Pseudomonas, e.g.,
Pseudomonas species antigen (e.g., ferredoxin reductase component)
Riboviria, e.g., Orthornavirae, e.g., Negarnaviricota, e.g., Monjiviricetes, e.g., Mononegavirales, e.g., Rhabdoviridae,
e.g., Lyssavirus, e.g., Rabies antigen
Riboviria, e.g., Orthornavirae, e.g., Negarnaviricota, e.g., Monjiviricetes, e.g., Mononegavirales, e.g., Pneumoviridae,
e.g., Orthopneumovirus, e.g., Respiratory syncytial virus (RSV) antigen
Riboviria, e.g., Orthornavirae, e.g., Negarnaviricota, e.g., Ellioviricetes, e.g., Bunyavirales, e.g., Phenuviridae, e.g.,
Phlebovirus, e.g., Rift Valley fever Virus antigen
Riboviria, e.g., Orthornavirae, e.g., Duplornaviricota, e.g., Resentoviricetes, e.g., Reovirales, e.g., Reoviridae, e.g.,
Sedoreovirinae, e.g., Rotavirus, e.g., Rotavirus antigen (e.g., VP8, H1N1 M2, H7N9 F)
Proteobacteria, e.g., Gammaproteobacteria, e.g., Enterobacterales, e.g., Enterobacteriaceae, e.., Salmonella, e.g.,
Salmonella typhi antigen (e.g., Vi polysaccharide)
Platyhelminthes, e.g., Diplostomida, e.g., Schistosomatidae, e.g., Schistosomatinae, e.g., Schistosoma, e.g.,
Schistosoma species antigen
Proteobacteria, e.g., Gammaproteobacteria, e.g., Enterobacterales, e.g., Enterobacteriaceae, e.g., Shigell, e.g., Shigella
antigen (e.g., shigella polysaccharides)
“Firmicutes”, e.g., Bacilli, e.g., Bacillales, e.g., Staphylococcaceae, e.g., Staphylococcus, e.g., Staphylococcal antigen
(e.g., staphylococcal enterotoxin B)
“Firmicutes”, e.g., Bacilli, e.g., Lactobacillales, e.g. Streptococcaceae, e.g., Streptococcus, e.g., Streptococcus
agalactiae antigen
“Firmicutes”, e.g., Bacilli, e.g., Lactobacillales, e.g., Streptococcaceae, e.g., Streptococcus, e.g., Streptococcus
pyogenes antigen
“Firmicutes”, e.g., Clostridia, e.g., Clostridiales, e.g., Clostridiaceae, e.g., Clostridium, e.g., Tetanus antigen (e.g.,
tetanus toxoid)
Riboviria, e.g., Orthornavirae, e.g., Kitrinoviricota, e.g., Flasuviricetes, e.g., Amarillovirales, e.g., Flaviviridae, e.g.,
Flavivirus, e.g., Tick-borne encephalitis (TBE) antigen
Apicomplexa, e.g., Conoidasida, e.g., Eucoccidiorida, e.g., Sarcocystidae, e.g., Toxoplasmatinae, e.g., Toxoplasma,
e.g., Toxoplasma gondii antigen
Spirochaetes, e.g., Spirochaetales, e.g., Spirochaetaceae, e.g., Treponema, e.g., Treponema pallidum antigen
“Actinobacteria”, e.g., Actinobacteria, e.g., Mycobacteriales, e.g., Mycobacteriaceae, e.g., Mycobacterium, e.g.,
Tuberculosis antigen
Riboviria, e.g., Orthornavirae, e.g., Negarnaviricota, e.g., Monjiviricetes, e.g., Mononegavirales, e.g., Rhabdoviridae,
e.g., Vesiculovirus, e.g., Vesicular stomatitis virus antigen (e.g., vesicular stomatitis virus glycoprotein)
Proteobacteria, e.g., Gammaproteobacteria, e.g., Vibrionales, e..g, Vibrionaceae, e.g., Vibrio, e.g., Vibrio cholerae
antigen
Riboviria, e.g., Orthornavirae, e.g., Kitrinoviricota, e.g., Flasuviricetes, e.g., Amarillovirales, e.g., Flaviviridae, e.g.,
Flavivirus, e.g., West Nile virus (WNV) antigen
Riboviria, e.g., Orthornavirae, e.g., Kitrinoviricota, e.g., Flasuviricetes, e.g., Amarillovirales, e.g., Flaviviridae, e.g.,
Flavivirus, e.g., Zika virus antigen
Apicomplexa, e.g., Aconoidasida, e.g., Haemospororida, e.g., Plasmodiidae, e.g., Plasmodium, e.g., Plasmodium vivax,
Plasmodium falciparum, Plasmodium malariae, Plasmodium ovale, or Plasmodium knowlesi.
Riboviria, e.g., Orthornavirae, e.g., Pisuviricota, e.g., Pisoniviricetes, e.g., Nidovirales, e.g., Coronaviridae, e.g.,
Betacoronavirus, e.g., Sebacovirus, e.g., severe acute respiratory syndrome-related coronavirus, e.g., SARS-CoV-2,
e.g., SARS-CoV-2 alpha variant, SARS-CoV-2 beta variant, SARS-CoV-2 gamma variant, SARS-CoV-2 delta variant,
or SARS-CoV-2 omicron variant

In some embodiments, a vaccine comprising an anellovector or anelloVLP as described herein is administered with an adjuvant. In certain embodiments, the adjuvant is an inorganic adjuvant (e.g., potassium alum, aluminium hydroxide, aluminium phosphate, or calcium phosphate hydroxide). In certain embodiments, the adjuvant is an oil-based adjuvant (e.g., paraffin oil). In certain embodiments, the adjuvant is a saponin. In certain embodiments, the adjuvant is a cytokine. In certain embodiments, the adjuvant is a squalene. In certain embodiments, the adjuvant is Freund's complete adjuvant.

In some embodiments, a vaccine as described herein is administered in a dose comprising about 10¹⁰ to 10¹⁴ viral genome equivalents of an anellovector as described herein. In some embodiments, a vaccine as described herein is administered as a dose comprising about 10¹⁰ to 10¹⁴ particles (e.g., anellovectors or anelloVLPs) as described herein.

### Ligands

In some instances, a surface moiety as described herein comprises a ligand (e.g., a ligand that binds specifically to a receptor on a target cell). In some embodiments, the ligand is a growth factor. In certain embodiments, the ligand binds to a growth factor receptor on the surface of the target cell. In some embodiments, the ligand is a cytokine. In some embodiments, the ligand is a hormone.

### II. Compositions and Methods for Making Anellovectors and AnelloVLPs

The present disclosure provides, in some aspects, anellovectors, anelloVLPs, and methods thereof for delivering effectors. In some embodiments, the anellovectors, anelloVLPs, or components thereof can be made as described below. In some embodiments, the compositions and methods described herein can be used to produce a genetic element or a genetic element construct. In some embodiments, the compositions and methods described herein can be used to produce one or more Anellovirus ORF molecules (e.g., an ORF1, ORF2, ORF2/2, ORF2/3, ORF1/1, or ORF1/2 molecule, or a functional fragment or splice variant thereof). In some embodiments, the compositions and methods described herein can be used to produce a proteinaceous exterior or a component thereof (e.g., an ORF1 molecule), e.g., in a host cell.

In some embodiments, the anellovectors, anelloVLPs, or components thereof can be made using a tandem construct, e.g., as described in PCT Application No. PCT/US2021/037091, which is incorporated herein by reference in its entirety.

The present disclosure provides, in some aspects, compositions (e.g., bacmids, donor vectors, baculovirus particles, and cells comprising same) and methods that can be used for producing anellovectors or anelloVLPs, e.g., as described herein. In some embodiments, the compositions and methods described herein can be used to produce a genetic element or a genetic element construct. In some embodiments, the compositions and methods described herein can be used to produce one or more Anellovirus ORF molecules (e.g., an ORF1, ORF2, ORF2/2, ORF2/3, ORF1/1, or ORF1/2 molecule, or a functional fragment or splice variant thereof). In some embodiments, the compositions and methods described herein can be used to produce a proteinaceous exterior or a component thereof (e.g., an ORF1 molecule). In some embodiments, the anellovectors, anelloVLPs, or components thereof can be made using a bacmid/insect cell system, e.g., as described as described in PCT Application No. PCT/US2021/037076, which is incorporated herein by reference in its entirety.

Without wishing to be bound by theory, rolling circle amplification may occur via Rep protein binding to a Rep binding site (e.g., comprising a 5′ UTR, e.g., comprising a hairpin loop and/or an origin of replication, e.g., as described herein) positioned 5′ relative to (or within the 5′ region of) the genetic element region. The Rep protein may then proceed through the genetic element region, resulting in the synthesis of the genetic element. The genetic element may then be circularized and then enclosed within a proteinaceous exterior to form an anellovector.

### Components and Assembly of Anellovectors and AnelloVLPs

The compositions and methods herein can be used to produce anellovectors and anelloVLPs. As described herein, an anellovector generally comprises a genetic element (e.g., a single-stranded, circular DNA molecule, e.g., comprising a 5′ UTR region as described herein) enclosed within a proteinaceous exterior (e.g., comprising a polypeptide encoded by an Anellovirus ORF1 nucleic acid, e.g., as described herein). In some embodiments, the genetic element comprises one or more sequences encoding Anellovirus ORFs (e.g., one or more of an Anellovirus ORF1, ORF2, ORF2/2, ORF2/3, ORF1/1, or ORF1/2). As described herein, an anelloVLP generally comprises a proteinaceous exterior (e.g., comprising a polypeptide encoded by an Anellovirus ORF1 nucleic acid, e.g., as described herein) and an exogenous effector. As used herein, an Anellovirus ORF or ORF molecule (e.g., an Anellovirus ORF1, ORF2, ORF2/2, ORF2/3, ORF1/1, or ORF1/2) includes a polypeptide comprising an amino acid sequence having at least 70%, 75%, 80%, 85%, 90%, 95%, 96%, 97%, 98%, 99%, or 100% sequence identity to a corresponding Anellovirus ORF sequence, e.g., as described herein or as described in PCT/US2018/037379 or PCT/US19/65995 (each of which is incorporated by reference herein in their entirety). In embodiments, the genetic element comprises a sequence encoding an Anellovirus ORF1, or a splice variant or functional fragment thereof (e.g., a jelly-roll region, e.g., as described herein). In some embodiments, the proteinaceous exterior comprises a polypeptide encoded by an Anellovirus ORF1 nucleic acid (e.g., an Anellovirus ORF1 molecule or a splice variant or functional fragment thereof).

In some embodiments, an anellovector is assembled by enclosing a genetic element (e.g., as described herein) within a proteinaceous exterior (e.g., as described herein). In some embodiments, an anellovector is assembled by enclosing a genetic element (e.g., as described herein) within a proteinaceous exterior in vitro (e.g., as described herein) (e.g., wherein the enclosing occurs external to a host cell, e.g., in the absence of a host cell). In some embodiments, the genetic element is enclosed within the proteinaceous exterior in a host cell (e.g., as described herein). In some embodiments, a surface moiety is attached to the proteinaceous exterior (e.g., as described herein). In some embodiments, the proteinaceous exterior comprises an ORF1 molecule comprising a surface moiety (e.g., as described herein). In certain embodiments, the ORF1 molecule comprises a surface moiety fused to an ORF1 domain.

In some embodiments, an anelloVLP is assembled by enclosing an exogenous effector within a proteinaceous exterior (e.g., as described herein) in vitro (e.g., wherein the enclosing occurs external to a host cell, e.g., in the absence of a host cell). In some embodiments, an anelloVLP is assembled by contacting a plurality of ORF1 molecules (e.g., as described herein) with an effector in vitro (e.g., wherein the contacting occurs external to a host cell, e.g., in the absence of a host cell). In some embodiments, an anelloVLP is assembled by attaching an exogenous effector to the exterior surface of a proteinaceous exterior (e.g., as described herein) in vitro. In some embodiments, a surface moiety is attached to the proteinaceous exterior (e.g., as described herein). In some embodiments, the proteinaceous exterior comprises an ORF1 molecule comprising a surface moiety (e.g., as described herein). In certain embodiments, the ORF1 molecule comprises a surface moiety fused to an ORF1 domain.

In some embodiments, a host cell expresses one or more polypeptides comprised in the proteinaceous exterior (e.g., a polypeptide encoded by an Anellovirus ORF1 nucleic acid, e.g., an ORF1 molecule). For example, in some embodiments, the host cell comprises a nucleic acid sequence encoding an Anellovirus ORF1 molecule, e.g., a splice variant or a functional fragment of an Anellovirus ORF1 polypeptide (e.g., a wild-type Anellovirus ORF1 protein or a polypeptide encoded by a wild-type Anellovirus ORF1 nucleic acid, e.g., as described herein). In embodiments, the nucleic acid sequence encoding the Anellovirus ORF1 molecule is comprised in a nucleic acid construct (e.g., a plasmid, viral vector, virus, minicircle, bacmid, or artificial chromosome) comprised in the host cell. In embodiments, the nucleic acid sequence encoding the Anellovirus ORF1 molecule is integrated into the genome of the host cell.

In some embodiments, a host cell produces a genetic element from a nucleic acid construct comprising the sequence of the genetic element. In some embodiments, the nucleic acid construct is selected from a plasmid, in vitro circularized nucleic acid molecule, viral nucleic acid molecule, minicircle, bacmid, or artificial chromosome. In some embodiments, the genetic element is excised from the nucleic acid construct and, optionally, converted from a double-stranded form to a single-stranded form (e.g., by denaturation). In some embodiments, the genetic element is generated by a polymerase based on a template sequence in the nucleic acid construct. In some embodiments, the polymerase produces a single-stranded copy of the genetic element sequence, which can optionally be circularized to form a genetic element as described herein. In other embodiments, the nucleic acid construct is a double-stranded minicircle produced by circularizing the nucleic acid sequence of the genetic element in vitro. In embodiments, the in vitro-circularized (IVC) minicircle is introduced into the host cell, where it is converted to a single-stranded genetic element suitable for enclosure in a proteinaceous exterior, as described herein.

In some embodiments, the host cell comprises a genetic element construct (e.g., a bacmid, plasmid, or minicircle). In some embodiments, the host cell comprises a bacmid comprising one or more sequences encoding Anellovirus ORF molecules (e.g., ORF1, ORF2, ORF2/2, ORF2/3, ORF1/1, and/or ORF1/2 ORF molecules), or functional fragments thereof. In some embodiments, proteinaceous exterior proteins are expressed from the bacmid. In embodiments, the proteinaceous exterior proteins expressed from the bacmid enclose a genetic element, thereby forming an anellovector. In some embodiments, the bacmid comprises a backbone suitable for replication of the nucleic acid construct in insect cells (e.g., Sf9 cells), e.g., a baculovirus backbone region. In some embodiments, the bacmid comprises a backbone region suitable for replication of the genetic element construct in bacterial cells (e.g., E. coli cells, e.g., DH 10Bac cells). In some embodiments, the genetic element construct comprises a backbone suitable for replication of the nucleic acid construct in insect cells (e.g., Sf9 cells), e.g., a baculovirus backbone region. In some embodiments, the genetic element construct comprises a backbone region suitable for replication of the genetic element construct in bacterial cells (e.g., E. coli cells, e.g., DH 10Bac cells). In some embodiments, the bacmid is introduced into the host cell via a baculovirus particle. In embodiments, the bacmid is produced by a producer cell, e.g., an insect cell (e.g., an Sf9 cell) or a bacterial cell (e.g., an E. coli cell, e.g., a DH 10Bac cell). In embodiments, the producer cell comprises a bacmid and/or a donor vector, e.g., as described herein. In embodiments, the producer cell further comprises sufficient cellular machinery for replication of the bacmid and/or donor vector.

ORF1 Molecules, e.g., for assembly of Anellovectors

An anellovector or anelloVLP as described herein generally comprises a proteinaceous exterior comprising a polypeptide encoded by an Anellovirus ORF1 nucleic acid (e.g., an Anellovirus ORF1 molecule or a splice variant or functional fragment thereof, e.g., as described herein). An ORF1 molecule may, in some embodiments, comprise one or more of: a first region comprising an arginine rich region, e.g., a region having at least 60% basic residues (e.g., at least 60%, 65%, 70%, 75%, 80%, 85%, 90%, 95%, or 100% basic residues; e.g., between 60%-90%, 60%-80%, 70%-90%, or 70-80% basic residues), and a second region comprising jelly-roll domain, e.g., at least six beta strands (e.g., 4, 5, 6, 7, 8, 9, 10, 11, or 12 beta strands). In embodiments, the proteinaceous exterior comprises one or more (e.g., 1, 2, 3, 4, or all 5) of an Anellovirus ORF1 arginine-rich region, jelly-roll region, N22 domain, hypervariable region, and/or C-terminal domain. In some embodiments, the proteinaceous exterior comprises an Anellovirus ORF1 jelly-roll region (e.g., as described herein). In some embodiments, the proteinaceous exterior comprises an Anellovirus ORF1 arginine-rich region (e.g., as described herein). In some embodiments, the proteinaceous exterior comprises an Anellovirus ORF1 N22 domain (e.g., as described herein). In some embodiments, the proteinaceous exterior comprises an Anellovirus hypervariable region (e.g., as described herein). In some embodiments, the proteinaceous exterior comprises an Anellovirus ORF1 C-terminal domain (e.g., as described herein).

In some embodiments, the anellovector comprises an ORF1 molecule and/or a nucleic acid encoding an ORF1 molecule. In some embodiments, the anelloVLP comprises an ORF1 molecule. Generally, an ORF1 molecule comprises a polypeptide having the structural features and/or activity of an Anellovirus ORF1 protein (e.g., an Anellovirus ORF1 protein as described herein), or a functional fragment thereof. In some embodiments, the ORF1 molecule comprises a truncation relative to an Anellovirus ORF1 protein (e.g., an Anellovirus ORF1 protein as described herein). In some embodiments, the ORF1 molecule is truncated by at least 10, 20, 30, 40, 50, 60, 70, 80, 90, 100, 150, 200, 250, 300, 350, 400, 450, 500, 550, 600, 650, or 700 amino acids of the Anellovirus ORF1 protein. In some embodiments, an ORF1 molecule comprises an amino acid sequence having at least 75%, 80%, 85%, 90%, 95%, 96%, 97%, 98%, or 99% sequence identity to a Betatorquevirus ORF1 protein, e.g., as described herein. An ORF1 molecule can generally bind to a nucleic acid molecule, such as DNA (e.g., a genetic element, e.g., as described herein). In some embodiments, an ORF1 molecule localizes to the nucleus of a cell. In certain embodiments, an ORF1 molecule localizes to the nucleolus of a cell.

Without wishing to be bound by theory, an ORF1 molecule may be capable of binding to other ORF1 molecules, e.g., to form a proteinaceous exterior (e.g., as described herein). Such an ORF1 molecule may be described as having the capacity to form a capsid. In some embodiments, the proteinaceous exterior may enclose a nucleic acid molecule (e.g., a genetic element as described herein, e.g., produced using a composition or construct as described herein) and/or an effector (e.g., an exogenous effector). In some embodiments, a plurality of ORF1 molecules may form a multimer, e.g., to produce a proteinaceous exterior. In some embodiments, the multimer may be a homomultimer. In other embodiments, the multimer may be a heteromultimer.

In some embodiments, a first plurality of anellovectors or anelloVLPs comprising an ORF1 molecule as described herein is administered to a subject. In some embodiments, a second plurality of anellovectors or anelloVLPs comprising an ORF1 molecule described herein, is subsequently administered to the subject following administration of the first plurality. In some embodiments the second plurality of anellovectors or anelloVLPs comprises an ORF1 molecule having the same amino acid sequence as the ORF1 molecule comprised by the anellovectors or anelloVLPs of the first plurality.

In some embodiments the second plurality of anellovectors or anelloVLPs comprises an ORF1 molecule having at least 70%, 75%, 80%, 85%, 90%, 95%, 96%, 97%, 98%, 99%, or 100% amino acid sequence identity to the ORF1 molecule comprised by the anellovectors or anelloVLPs of the first plurality.

### ORF2 Molecules, e.g., for Assembly of Anellovectors

Producing an anellovector or anelloVLP using the compositions or methods described herein may involve expression of an Anellovirus ORF2 molecule (e.g., as described herein), or a splice variant or functional fragment thereof. In some embodiments, the anellovector comprises an ORF2 molecule, or a splice variant or functional fragment thereof, and/or a nucleic acid encoding an ORF2 molecule, or a splice variant or functional fragment thereof. In some embodiments, the anellovector or anelloVLP does not comprise an ORF2 molecule, or a splice variant or functional fragment thereof, and/or a nucleic acid encoding an ORF2 molecule, or a splice variant or functional fragment thereof. In some embodiments, producing the anellovector or anelloVLP comprises expression of an ORF2 molecule, or a splice variant or functional fragment thereof, but the ORF2 molecule is not incorporated into the anellovector or anelloVLP.

### Production of Protein Components

Protein components of an anellovector or anelloVLP, e.g., ORF1, can be produced in a variety of ways, e.g., as described herein. In some embodiments, one or more protein components of an anellovector or anelloVLP, including, e.g., the proteinaceous exterior, are produced in a host cell (e.g., the same host cell that packages the genetic elements into the proteinaceous exteriors, thereby producing the anellovectors). In some embodiments, one or more protein components of an anellovector or anelloVLP, including, e.g., the proteinaceous exterior, are produced in a cell that does not comprise a genetic element and/or a genetic element construct (e.g., as described herein). In some embodiments, one or more protein components of an anellovector or anelloVLP are produced and then secreted from a host cell. In some embodiments, one or more protein components of an anellovector or anelloVLP are produced and then isolated from a host cell (e.g., by lysing the host cell).

### Baculovirus Expression Systems

A viral expression system, e.g., a baculovirus expression system, may be used to express proteins (e.g., for production of anellovectors or anelloVLPs), e.g., as described herein. Baculoviruses are rod-shaped viruses with a circular, supercoiled double-stranded DNA genome. Genera of baculoviruses include: Alphabaculovirus (nucleopolyhedroviruses (NPVs) isolated from Lepidoptera), Betabaculoviruses (granuloviruses (GV) isolated from Lepidoptera), Gammabaculoviruses (NPVs isolated from Hymenoptera) and Deltabaculoviruses (NPVs isolated from Diptera). While GVs typically contain only one nucleocapsid per envelope, NPVs typically contain either single (SNPV) or multiple (MNPV) nucleocapsids per envelope. The enveloped virions are further occluded in granulin matrix in GVs and polyhedrin in NPVs. Baculoviruses typically have both lytic and occluded life cycles. In some embodiments, the lytic and occluded life cycles manifest independently throughout the three phases of virus replication: early, late, and very late phase. In some embodiments, during the early phase, viral DNA replication takes place following viral entry into the host cell, early viral gene expression and shut-off of the host gene expression machinery. In some embodiments, in the late phase late genes that code for viral DNA replication are expressed, viral particles are assembled, and extracellular virus (EV) is produced by the host cell. In some embodiments, in the very late phase the polyhedrin and p10 genes are expressed, occluded viruses (OV) are produced by the host cell, and the host cell is lysed. Since baculoviruses infect insect species, they can be used as biological agents to produce exogenous proteins in baculoviruses-permissive insect cells or larvae. Different isolates of baculovirus, such as Autographa californica multiple nuclear polyhedrosis virus (AcMNPV) and Bombyx mori (silkworm) nuclear polyhedrosis virus (BmNPV) may be used in exogenous protein expression. Various baculoviral expression systems are commercially available, e.g., from ThermoFisher.

In some embodiments, the proteins described herein (e.g., an Anellovirus ORF molecule, e.g., ORF1, ORF2, ORF2/2, ORF2/3, ORF1/1, or ORF1/2, or a functional fragment or splice variant thereof) may be expressed using a baculovirus expression vector (e.g., a bacmid) that comprises one or more components described herein. For example, a baculovirus expression vector may include one or more of (e.g., all of) a selectable marker (e.g., kanR), an origin of replication (e.g., one or both of a bacterial origin of replication and an insect cell origin of replication), a recombinase recognition site (e.g., an att site), and a promoter. In some embodiments, a baculovirus expression vector (e.g., a bacmid as described herein) can be produced by replacing the naturally occurring wild-type polyhedrin gene, which encodes for baculovirus occlusion bodies, with genes encoding the proteins described herein. In some embodiments, the genes encoding the proteins described herein are cloned into a baculovirus expression vector (e.g., a bacmid as described herein) containing a baculovirus promoter. In some embodiments, the baculovirual vector comprises one or more non-baculoviral promoters, e.g., a mammalian promoter or an Anellovirus promoter. In some embodiments, the genes encoding the proteins described herein are cloned into a donor vector (e.g., as described herein), which is then contacted with an empty baculovirus expression vector (e.g., an empty bacmid) such that the genes encoding the proteins described herein are transferred (e.g., by homologous recombination or transposase activity) from the donor vector into the baculovirus expression vector (e.g., bacmid). In some embodiments, the baculovirus promoter is flanked by baculovirus DNA from the nonessential polyhedrin gene locus. In some embodiments, a protein described herein is under the transcriptional control of the AcNPV polyhedrin promoter in the very late phase of viral replication. In some embodiments, a strong promoter suitable for use in baculoviral expression in insect cells include, but are not limited to, baculovirus p10 promoters, polyhedrin (polh) promoters, p6.9 promoters and capsid protein promoters. Weak promoters suitable for use in baculoviral expression in insect cells include iel, ie2, ie0, etI, 39K (aka pp31) and gp64 promoters of baculoviruses.

In some embodiments, a recombinant baculovirus is produced by homologous recombination between a baculoviral genome (e.g., a wild-type or mutant baculoviral genome), and a transfer vector. In some embodiments, one or more genes encoding a protein described herein are cloned into the transfer vector. In some embodiments, the transfer vector further contains a baculovirus promoter flanked by DNA from a nonessential gene locus, e.g., polyhedrin gene. In some embodiments, one or more genes encoding a protein described herein are inserted into the baculoviral genome by homologous recombination between the baculoviral genome and the transfer vector. In some embodiments, the baculoviral genome is linearized at one or more unique sites. In some embodiments, the linearized sites are located near the target site for insertion of genes encoding the proteins described herein into the baculoviral genome. In some embodiments, a linearized baculoviral genome missing a fragment of the baculoviral genome downstream from a gene, e.g., polyhedrin gene, can be used for homologous recombination. In some embodiments, the baculoviral genome and transfer vector are co-transfected into insect cells. In some embodiments, the method of producing the recombinant baculovirus comprises the steps of preparing the baculoviral genome for performing homologous recombination with a transfer vector containing the genes encoding one or more protein described herein and co-transfecting the transfer vector and the baculoviral genome DNA into insect cells. In some embodiments, the baculoviral genome comprises a region homologous to a region of the transfer vector. These homologous regions may enhance the probability of recombination between the baculoviral genome and the transfer vector. In some embodiments, the homology region in the transfer vector is located upstream or downstream of the promoter. In some embodiments, to induce homologous recombination, the baculoviral genome, and transfer vector are mixed at a weight ratio of about 1:1 to 10:1.

In some embodiments, a recombinant baculovirus is generated by a method comprising site-specific transposition with Tn7, e.g., whereby the genes encoding the proteins described herein are inserted into bacmid DNA, e.g., propagated in bacteria, e.g., E. coli (e.g., DH 10Bac cells). In some embodiments, the genes encoding the proteins described herein are cloned into a pFASTBAC® vector and transformed into competent cells, e.g., DH1OBAC® competent cells, containing the bacmid DNA with a mini-attTn7 target site. In some embodiments, the baculovirus expression vector, e.g., pFASTBAC® vector, may have a promoter, e.g., a dual promoter (e.g., polyhedrin promoter, p10 promoter). Commercially available pFASTBAC® donor plasmids include: pFASTBAC 1, pFASTBAC HT, and pFASTBAC DUAL. In some embodiments, recombinant bacmid DNA containing-colonies are identified and bacmid DNA is isolated to transfect insect cells.

In some embodiments, a baculoviral vector is introduced into an insect cell together with a helper nucleic acid. The introduction may be concurrent or sequential. In some embodiments, the helper nucleic acid provides one or more baculoviral proteins, e.g., to promote packaging of the baculoviral vector. In some embodiments, recombinant baculovirus produced in insect cells (e.g., by homologous recombination) is expanded and used to infect insect cells (e.g., in the mid-logarithmic growth phase) for recombinant protein expression. In some embodiments, recombinant bacmid DNA produced by site-specific transposition in bacteria, e.g., E. coli, is used to transfect insect cells with a transfection agent, e.g., Cellfectin® II. Additional information on baculovirus expression systems is discussed in U.S. patent application Ser. Nos. 14/447,341, 14/277,892, and 12/278,916, which are hereby incorporated by reference.

### Insect Cell Systems

The proteins described herein may be expressed in host cells (e.g., insect cells) infected or transfected with recombinant baculovirus or bacmid DNA, e.g., as described above. In some embodiments, the host or host cell is an insect cell (e.g., an Sf9 cell, Sf21 cell, or Hi5 cell). In some embodiments, the insect cell is derived from Bombyx mori, Mamestra brassicae, Spodopterafrugiperda, Trichoplusia ni, or Drosophila melanogaster. In some embodiments, the insect cell is selected from Sf9 and Sf21 cells derived from Spodopterafrugiperda and Tn-368 and High Five™ BTI-TN-5B1-4 cells (also referred to as Hi5 cells) derived from Trichoplusia ni. In some embodiments, insect cell lines Sf21 and Sf9, derived from the ovaries of the pupal fall army worm Spodopterafrugiperda, can be used for the expression of recombinant proteins using the baculovirus expression system. In some embodiments, Sf21 and Sf9 insect cells may be cultured in commercially available serum-supplemented or serum-free media. Suitable media for culturing insect cells include: Grace's Supplemented (TNM-FH), IPL-41, TC-100, Schneider's Drosophila, SF-900 II SFM, and EXPRESS-FIVE™ SFM. In some embodiments, some serum-free media formulations utilize a phosphate buffer system to maintain a culture pH in the range of 6.0-6.4 (Licari et al. Insect cell hosts for baculovirus expression vectors contain endogenous exoglycosidase activity. Biotechnology Progress 9: 146-152 (1993) and Drugmand et al. Insect cells as factories for biomanufacturing. Biotechnology Advances 30:1140-1157 (2012)) for both cultivation and recombinant protein production. In some embodiments, a pH of 6.0-6.8 for cultivating various insect cell lines may be used. In some embodiments, insect cells are cultivated in suspension or as a monolayer at a temperature between 25° to 30° C. with aeration. Additional information on insect cells is discussed, for example, in U.S. patent application Ser. Nos. 14/564,512 and 14/775,154, each of which is hereby incorporated by reference.

### Mammalian Cell Systems

In some embodiments, the proteins described herein may be expressed in vitro in animal cell lines infected or transfected with a vector encoding the protein, e.g., as described herein. Animal cell lines envisaged in the context of the present disclosure include porcine cell lines, e.g., immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines suitable for expressing the proteins described herein include, but are not limited to cell lines of human or primate origin, such as human or primate kidney carcinoma cell lines.

### Genetic Element Constructs, e.g., for Assembly of Anellovectors

The genetic element of an anellovector as described herein may be produced from a genetic element construct that comprises a genetic element region and optionally other sequence such as vector backbone. Generally, the genetic element construct comprises an Anellovirus 5′ UTR (e.g., as described herein). A genetic element construct may be any nucleic acid construct suitable for delivery of the sequence of the genetic element into a host cell in which the genetic element can be enclosed within a proteinaceous exterior. In some embodiments, the genetic element construct comprises a promoter. In some embodiments, the genetic element construct is a linear nucleic acid molecule. In some embodiments, the genetic element construct is a circular nucleic acid molecule (e.g., a plasmid, bacmid, or a minicircle, e.g., as described herein). The genetic element construct may, in some embodiments, be double-stranded. In other embodiments, the genetic element is single-stranded. In some embodiments, the genetic element construct comprises DNA. In some embodiments, the genetic element construct comprises RNA. In some embodiments, the genetic element construct comprises one or more modified nucleotides.

In some aspects, the present disclosure provides a method for replication and propagation of the anellovector as described herein (e.g., in a cell culture system), which may comprise one or more of the following steps: (a) introducing (e.g., transfecting) a genetic element (e.g., linearized) into a cell line sensitive to anellovector infection; (b) harvesting the cells and optionally isolating cells showing the presence of the genetic element; (c) culturing the cells obtained in step (b) (e.g., for at least three days, such as at least one week or longer), depending on experimental conditions and gene expression; and (d) harvesting the cells of step (c), e.g., as described herein.

### Plasmids

In some embodiments, the genetic element construct is a plasmid. The plasmid will generally comprise the sequence of a genetic element as described herein as well as an origin of replication suitable for replication in a host cell (e.g., a bacterial origin of replication for replication in bacterial cells) and a selectable marker (e.g., an antibiotic resistance gene). In some embodiments, the sequence of the genetic element can be excised from the plasmid. In some embodiments, the plasmid is capable of replication in a bacterial cell. In some embodiments, the plasmid is capable of replication in a mammalian cell (e.g., a human cell). In some embodiments, a plasmid is at least 300, 400, 500, 600, 700, 800, 900, 1000, 2000, 3000, 4000, or 5000 bp in length. In some embodiments, the plasmid is less than 600, 700, 800, 900, 1000, 2000, 3000, 4000, 5000, 6000, 7000, 8000, 9000, or 10,000 bp in length. In some embodiments, the plasmid has a length between 300-400, 400-500, 500-600, 600-700, 700-800, 800-900, 900-1000, 1000-1500, 1500-2000, 2000-2500, 2500-3000, 3000-4000, or 4000-5000 bp. In some embodiments, the genetic element can be excised from a plasmid (e.g., by in vitro circularization), for example, to form a minicircle, e.g., as described herein. In embodiments, excision of the genetic element separates the genetic element sequence from the plasmid backbone (e.g., separates the genetic element from a bacterial backbone).

### Small Circular Nucleic Acid Constructs

In some embodiments, the genetic element construct is a circular nucleic acid construct, e.g., lacking a backbone (e.g., lacking a bacterial origin of replication and/or selectable marker). In embodiments, the genetic element is a double-stranded circular nucleic acid construct. In embodiments, the double-stranded circular nucleic acid construct is produced by in vitro circularization (IVC), e.g., as described herein. In embodiments, the double-stranded circular nucleic acid construct can be introduced into a host cell, in which it can be converted into or used as a template for generating single-stranded circular genetic elements, e.g., as described herein. In some embodiments, the circular nucleic acid construct does not comprise a plasmid backbone or a functional fragment thereof. In some embodiments, the circular nucleic acid construct is at least 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, or 4500 bp in length. In some embodiments, the circular nucleic acid construct is less than 2900, 3000, 3100, 3200, 3300, 3400, 3500, 3600, 3700, 3800, 3900, 4000, 4100, 4200, 4300, 4400, 4500, 4600, 4700, 4800, 4900, 5000, 5500, or 6000 bp in length. In some embodiments, the circular nucleic acid construct is between 2000-2100, 2100-2200, 2200-2300, 2300-2400, 2400-2500, 2500-2600, 2600-2700, 2700-2800, 2800-2900, 2900-3000, 3000-3100, 3100-3200, 3200-3300, 3300-3400, 3400-3500, 3500-3600, 3600-3700, 3700-3800, 3800-3900, 3900-4000, 4000-4100, 4100-4200, 4200-4300, 4300-4400, or 4400-4500 bp in length. In some embodiments, the circular nucleic acid construct is a minicircle.

### In Vitro Circularization

In some instances, the genetic element to be packaged into a proteinaceous exterior is a single stranded circular DNA. The genetic element may, in some instances, be introduced into a host cell via a genetic element construct having a form other than a single stranded circular DNA. For example, the genetic element construct may be a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or TraI, e.g., as described in Wawrzyniak et al. 2017, Front. Microbiol. 8: 2353; incorporated herein by reference with respect to the listed enzymes). In some embodiments, the double-stranded circular DNA is produced by in vitro circularization (IVC), e.g., as described in Example 15 or PCT Publication No. WO 2020/123816, incorporated by reference herein in its entirety.

Generally, in vitro circularized DNA constructs can be produced by digesting a genetic element construct (e.g., a plasmid comprising the sequence of a genetic element) to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an anellovector, e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

### Tandem Constructs

In some embodiments, a genetic element construct comprises a first copy of a genetic element sequence (e.g., the nucleic acid sequence of a genetic element, e.g., as described herein) and at least a portion of a second copy of a genetic element sequence (e.g., the nucleic acid sequence of the same genetic element, or the nucleic acid sequence of a different genetic element), arranged in tandem. Genetic element constructs having such a structure are generally referred to herein as tandem constructs. Such tandem constructs are used for producing an anellovector genetic element. The first copy of the genetic element sequence and the second copy of the genetic element sequence may, in some instances, be immediately adjacent to each other on the genetic acid construct. In other instances, the first copy of the genetic element sequence and the second copy of the genetic element sequence may be separated, e.g., by a spacer sequence. In some embodiments, the second copy of the genetic element sequence, or the portion thereof, comprises an upstream replication-facilitating sequence (uRFS), e.g., as described herein. In some embodiments, the second copy of the genetic element sequence, or the portion thereof, comprises a downstream replication-facilitating sequence (dRFS), e.g., as described herein. In some embodiments, the uRFS and/or dRFS comprises an origin of replication (e.g., a mammalian origin of replication, an insect origin of replication, or a viral origin of replication, e.g., a non-Anellovirus origin of replication, e.g., as described herein) or portion thereof. In some embodiments, the uRFS and/or dRFS does not comprise an origin of replication. In some embodiments, the uRFS and/or dRFS comprises a hairpin loop (e.g., in the 5′ UTR). In some embodiments, a tandem construct produces higher levels of a genetic element than an otherwise similar construct lacking the second copy of the genetic element or portion thereof. Without being bound by theory, a tandem construct described herein may, in some embodiments, replicate by rolling circle replication. In some embodiments, a tandem construct is a plasmid. In some embodiments, a tandem construct is circular. In some embodiments, a tandem construct is linear. In some embodiments, a tandem construct is single-stranded. In some embodiments, a tandem construct is double-stranded. In some embodiments, a tandem construct is DNA.

A tandem construct may, in some instances, include a first copy of the sequence of the genetic element and a second copy of the sequence of the genetic element, or a portion thereof. It is understood that the second copy can be an identical copy of the first copy or a portion thereof, or can comprise one or more sequence differences, e.g., substitutions, additions, or deletions. In some instances, the second copy of the genetic element sequence or portion thereof is positioned 5′ relative to the first copy of the genetic element sequence. In some instances, the second copy of the genetic element sequence or portion thereof is positioned 3′ relative to the first copy of the genetic element sequence. In some instances, the second copy of the genetic element sequence or portion thereof and the first copy of the genetic element sequence are adjacent to each other in the tandem construct. In some instances, the second copy of the genetic element sequence or portion thereof and the first copy of the genetic element sequence are separated, e.g., by a spacer sequence.

In some embodiments, the tandem constructs described herein can be used to produce the genetic element of a vector (e.g., anellovector), vehicle, or particle (e.g., viral particle) comprising a capsid (e.g., a capsid comprising an Anellovirus ORF, e.g., an ORF1 molecule, e.g., as described herein) encapsulating a genetic element comprising a protein binding sequence that binds to the capsid and a heterologous (e.g., relative to the Anellovirus from which the ORF1 molecule was derived) sequence encoding a therapeutic effector. In embodiments, the vector is capable of delivering the genetic element into a mammalian, e.g., human, cell. In some embodiments, the genetic element has less than about 50% (e.g., less than 50%, 40%, 30%, 25%, 20%, 15%, 10%, 9%, 8%, 7%, 6%, 5.5%, 5%, 4.5%, 4%, 3.5%, 3%, 2.5%, 2%, 1.5%, or less) identity to a wild type Anellovirus genome sequence. In some embodiments, the genetic element has no more than 1.5%, 2%, 2.5%, 3%, 3.5%, 4%, 4.5%, 5%, 5.5%, 6%, 7%, 8%, 9%, 10%, 15%, 20%, 25%, 30%, 40%, 50%, 60%, 70%, 75%, or 80% identity to a wild type Anellovirus genome sequence. In some embodiments, the genetic element has greater than about 2000, 3000, 4000, 4500, or 5000 contiguous nucleotides of non-Anellovirus genome sequence. In some embodiments, the genetic element has greater than about 2000 to 5000, 2500 to 4500, 3000 to 4500, 2500 to 4500, 3500, or 4000, 4500 (e.g., between about 3000 to 4500) nucleotides nucleotides of non-Anellovirus genome sequence.

In some embodiments of the systems and methods herein, a vector (e.g., an anellovector) is made by introducing into a cell a first nucleic acid molecule that is a genetic element or genetic element construct, e.g., a tandem construct, and a second nucleic acid molecule encoding one or more additional proteins (e.g., a Rep molecule and/or a capsid protein), e.g., as described herein. In some embodiments, the first nucleic acid molecule and the second nucleic acid molecule are attached to each other (e.g., in a genetic element construct described herein, e.g., in cis). In some embodiments, the first nucleic acid molecule and the second nucleic acid molecule are separate (e.g, in trans). In some embodiments, the first nucleic acid molecule is a plasmid, cosmid, bacmid, minicircle, or artificial chromosome. In some embodiments, the second nucleic acid molecule is a plasmid, cosmid, bacmid, minicircle, or artificial chromosome. In some embodiments, the second nucleic acid molecule is integrated into the genome of the host cell.

In some embodiments, the method further includes introducing the first nucleic acid molecule and/or the second nucleic acid molecule into the host cell. In some embodiments, the second nucleic acid molecule is introduced into the host cell prior to, concurrently with, or after the first nucleic acid molecule. In other embodiments, the second nucleic acid molecule is integrated into the genome of the host cell. In some embodiments, the second nucleic acid molecule is or comprises or is part of a helper construct, helper virus or other helper vector, e.g., as described herein.

### Cis/Trans Constructs

In some embodiments, a genetic element construct as described herein comprises one or more sequences encoding one or more Anellovirus ORFs, e.g., proteinaceous exterior components (e.g., polypeptides encoded by an Anellovirus ORF1 nucleic acid, e.g., as described herein). For example, the genetic element construct may comprise a nucleic acid sequence encoding an Anellovirus ORF1 molecule. Such genetic element constructs can be suitable for introducing the genetic element and the Anellovirus ORF(s) into a host cell in cis. In other embodiments, a genetic element construct as described herein does not comprise sequences encoding one or more Anellovirus ORFs, e.g., proteinaceous exterior components (e.g., polypeptides encoded by an Anellovirus ORF1 nucleic acid, e.g., as described herein). For example, the genetic element construct may not comprise a nucleic acid sequence encoding an Anellovirus ORF1 molecule. Such genetic element constructs can be suitable for introducing the genetic element into a host cell, with the one or more Anellovirus ORFs to be provided in trans (e.g., via introduction of a second nucleic acid construct encoding one or more of the Anellovirus ORFs, or via an Anellovirus ORF cassette integrated into the genome of the host cell). In some embodiments, an ORF1 molecule is provided in trans, e.g., as described herein. In some embodiments, an ORF2 molecule is provided in trans, e.g., as described herein. In some embodiments, an ORF1 molecule and an ORF1 molecule are both provided in trans, e.g., as described herein.

In some embodiments, the genetic element construct comprises a sequence encoding an Anellovirus ORF1 molecule, or a splice variant or functional fragment thereof (e.g., a jelly-roll region, e.g., as described herein). In embodiments, the portion of the genetic element that does not comprise the sequence of the genetic element comprises the sequence encoding the Anellovirus ORF1 molecule, or splice variant or functional fragment thereof (e.g., in a cassette comprising a promoter and the sequence encoding the Anellovirus ORF1 molecule, or splice variant or functional fragment thereof). In further embodiments, the portion of the construct comprising the sequence of the genetic element comprises a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-component anellovector (e.g., an anellovector that upon infecting a cell, enables the cell to produce additional copies of the anellovector without introducing further nucleic acid constructs, e.g., encoding one or more Anellovirus ORFs as described herein, into the cell).

In other embodiments, the genetic element does not comprise a sequence encoding an Anellovirus ORF1 molecule, or a splice variant or functional fragment thereof (e.g., a jelly-roll region, e.g., as described herein). In embodiments, enclosure of such a genetic element in a proteinaceous exterior (e.g., as described herein) produces a replication-incompetent anellovector (e.g., an anellovector that, upon infecting a cell, does not enable the infected cell to produce additional anellovectors, e.g., in the absence of one or more additional constructs, e.g., encoding one or more Anellovirus ORFs as described herein).

### Expression Cassettes

In some embodiments, a genetic element construct comprises one or more cassettes for expression of a polypeptide or noncoding RNA (e.g., a miRNA or an siRNA). In some embodiments, the genetic element construct comprises a cassette for expression of an effector (e.g., an exogenous or endogenous effector), e.g., a polypeptide or noncoding RNA, as described herein. In some embodiments, the genetic element construct comprises a cassette for expression of an Anellovirus protein (e.g., an Anellovirus ORF1, ORF2, ORF2/2, ORF2/3, ORF1/1, or ORF1/2, or a functional fragment thereof). The expression cassettes may, in some embodiments, be located within the genetic element sequence. In embodiments, an expression cassette for an effector is located within the genetic element sequence. In embodiments, an expression cassette for an Anellovirus protein is located within the genetic element sequence. In other embodiments, the expression cassettes are located at a position within the genetic element construct outside of the sequence of the genetic element (e.g., in the backbone). In embodiments, an expression cassette for an Anellovirus protein is located at a position within the genetic element construct outside of the sequence of the genetic element (e.g., in the backbone).

A polypeptide expression cassette generally comprises a promoter and a coding sequence encoding a polypeptide, e.g., an effector (e.g., an exogenous or endogenous effector as described herein) or an Anellovirus protein (e.g., a sequence encoding an Anellovirus ORF1, ORF2, ORF2/2, ORF2/3, ORF1/1, or ORF1/2, or a functional fragment thereof). Exemplary promoters that can be included in an polypeptide expression cassette (e.g., to drive expression of the polypeptide) include, without limitation, constitutive promoters (e.g., CMV, RSV, PGK, EF1a, or SV40), cell or tissue-specific promoters (e.g., skeletal α-actin promoter, myosin light chain 2A promoter, dystrophin promoter, muscle creatine kinase promoter, liver albumin promoter, hepatitis B virus core promoter, osteocalcin promoter, bone sialoprotein promoter, CD2 promoter, immunoglobulin heavy chain promoter, T cell receptor a chain promoter, neuron-specific enolase (NSE) promoter, or neurofilament light-chain promoter), and inducible promoters (e.g., zinc-inducible sheep metallothionine (MT) promoter; the dexamethasone (Dex)-inducible mouse mammary tumor virus (MMTV) promoter; the T7 polymerase promoter system, tetracycline-repressible system, tetracycline-inducible system, RU486-inducible system, rapamycin-inducible system), e.g., as described herein. In some embodiments, the expression cassette further comprises an enhancer, e.g., as described herein.

### Design and Production of a Genetic Element Construct

Various methods are available for synthesizing a genetic element construct. For instance, the genetic element construct sequence may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the genetic element construct. The segments or ORFs may be assembled into the genetic element construct, e.g., by in vitro recombination or unique restriction sites at 5′ and 3′ ends to enable ligation.

The genetic element construct can be synthesized with a design algorithm that parses the construct sequence into oligo-length fragments, creating suitable design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element construct or segment of the genetic element construct may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. In some embodiments, shotgun sequencing is performed.

A genetic element construct can be designed such that factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, e.g., as described herein. In some embodiments, replication and/or packaging signals can be incorporated into a genetic element, for example, to induce amplification and/or encapsulation. In some embodiments, an effector is inserted into a specific site in the genome. In some embodiments, one or more viral ORFs are replaced with an effector.

In another example, when replication or packaging factors are supplied in trans, the genetic element may lack genes encoding one or more of an Anellovirus ORF1, ORF1/1, ORF1/2, ORF2, ORF2/2, ORF2/3, or ORF2t/3, e.g., as described herein; this protein or proteins may be supplied, e.g., by another nucleic acid, e.g., a helper nucleic acid. In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

In some embodiments, the genetic element construct may be designed using computer-aided design tools.

General methods of making constructs are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

### Effectors

The compositions and methods described herein can be used to produce a genetic element of an anellovector comprising a sequence encoding an effector (e.g., an exogenous effector or an endogenous effector), e.g., as described herein. The compositions and methods described herein can also be used to produce an anelloVLP comprising an effector (e.g., an exogenous effector or an endogenous effector), e.g., as described herein. The effector may be, in some instances, an endogenous effector or an exogenous effector. In some embodiments, the effector is a therapeutic effector. In some embodiments, the effector comprises a polypeptide (e.g., a therapeutic polypeptide or peptide, e.g., as described herein). In some embodiments, the effector comprises a non-coding RNA (e.g., an miRNA, siRNA, shRNA, mRNA, lncRNA, RNA, DNA, antisense RNA, or gRNA). In some embodiments, the effector comprises a regulatory nucleic acid, e.g., as described herein.

In some embodiments, the effector-encoding sequence may be inserted into the genetic element e.g., at a non-coding region, e.g., a noncoding region disposed 3′ of the open reading frames and 5′ of the GC-rich region of the genetic element, in the 5′ noncoding region upstream of the TATA box, in the 5′ UTR, in the 3′ noncoding region downstream of the poly-A signal, or upstream of the GC-rich region. In some embodiments, the effector-encoding sequence may be inserted into the genetic element, e.g., in a coding sequence (e.g., in a sequence encoding an Anellovirus ORF1, ORF1/1, ORF1/2, ORF2, ORF2/2, ORF2/3, and/or ORF2t/3, e.g., as described herein). In some embodiments, the effector-encoding sequence replaces all or a part of the open reading frame. In some embodiments, the genetic element comprises a regulatory sequence (e.g., a promoter or enhancer, e.g., as described herein) operably linked to the effector-encoding sequence.

### Host Cells

The anellovectors described herein can be produced, for example, in a host cell. Generally, a host cell is provided that comprises an anellovector genetic element and the components of an anellovector proteinaceous exterior (e.g., a polypeptide encoded by an Anellovirus ORF1 nucleic acid or an Anellovirus ORF1 molecule). The host cell is then incubated under conditions suitable for enclosure of the genetic element within the proteinaceous exterior (e.g., culture conditions as described herein). In some embodiments, the host cell is further incubated under conditions suitable for release of the anellovector from the host cell, e.g., into the surrounding supernatant. In some embodiments, the host cell is lysed for harvest of anellovectors from the cell lysate. In some embodiments, an anellovector may be introduced to a host cell line grown to a high cell density. In some embodiments, a host cell is an Expi-293 cell.

Introduction of Genetic Elements into Host Cells

The genetic element, or a nucleic acid construct comprising the sequence of a genetic element, may be introduced into a host cell. In some embodiments, the genetic element itself is introduced into the host cell. In some embodiments, a genetic element construct comprising the sequence of the genetic element (e.g., as described herein) is introduced into the host cell. A genetic element or genetic element construct can be introduced into a host cell, for example, using methods known in the art. For example, a genetic element or genetic element construct can be introduced into a host cell by transfection (e.g., stable transfection or transient transfection). In embodiments, the genetic element or genetic element construct is introduced into the host cell by lipofectamine transfection. In embodiments, the genetic element or genetic element construct is introduced into the host cell by calcium phosphate transfection. In some embodiments, the genetic element or genetic element construct is introduced into the host cell by electroporation. In some embodiments, the genetic element or genetic element construct is introduced into the host cell using a gene gun. In some embodiments, the genetic element or genetic element construct is introduced into the host cell by nucleofection. In some embodiments, the genetic element or genetic element construct is introduced into the host cell by PEI transfection. In some embodiments, the genetic element is introduced into the host cell by contacting the host cell with an anellovector comprising the genetic element

In embodiments, the genetic element construct is capable of replication once introduced into the host cell. In embodiments, the genetic element can be produced from the genetic element construct once introduced into the host cell. In some embodiments, the genetic element is produced in the host cell by a polymerase, e.g., using the genetic element construct as a template.

In some embodiments, the genetic elements or vectors comprising the genetic elements are introduced (e.g., transfected) into cell lines that express a viral polymerase protein in order to achieve expression of the anellovector. To this end, cell lines that express an anellovector polymerase protein may be utilized as appropriate host cells. Host cells may be similarly engineered to provide other viral functions or additional functions.

To prepare the anellovector disclosed herein, a genetic element construct may be used to transfect cells that provide anellovector proteins and functions required for replication and production. Alternatively, cells may be transfected with a second construct (e.g., a virus) providing anellovector proteins and functions before, during, or after transfection by the genetic element or vector comprising the genetic element disclosed herein. In some embodiments, the second construct may be useful to complement production of an incomplete viral particle. The second construct (e.g., virus) may have a conditional growth defect, such as host range restriction or temperature sensitivity, e.g., which allows the subsequent selection of transfectant viruses. In some embodiments, the second construct may provide one or more replication proteins utilized by the host cells to achieve expression of the anellovector. In some embodiments, the host cells may be transfected with vectors encoding viral proteins such as the one or more replication proteins. In some embodiments, the second construct comprises an antiviral sensitivity.

The genetic element or vector comprising the genetic element disclosed herein can, in some instances, be replicated and produced into anellovectors using techniques known in the art. For example, various viral culture methods are described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

### Methods for Providing Protein(s) in Cis or Trans

In some embodiments (e.g., cis embodiments described herein), the genetic element construct further comprises one or more expression cassettes comprising a coding sequence for an Anellovirus ORF (e.g., an Anellovirus ORF1, ORF2, ORF2/2, ORF2/3, ORF1/1, or ORF1/2, or a functional fragment thereof). In embodiments, the genetic element construct comprises an expression cassette comprising a coding sequence for an Anellovirus ORF1, or a splice variant or functional fragment thereof. Such genetic element constructs, which comprise expression cassettes for the effector as well as the one or more Anellovirus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis anellovector production methods in host cells, e.g., as described herein.

In some embodiments (e.g., trans embodiments described herein), the genetic element does not comprise an expression cassette comprising a coding sequence for one or more Anellovirus ORFs (e.g., an Anellovirus ORF1, ORF2, ORF2/2, ORF2/3, ORF1/1, or ORF1/2, or a functional fragment thereof). In embodiments, the genetic element construct does not comprise an expression cassette comprising a coding sequence for an Anellovirus ORF1, or a splice variant or functional fragment thereof. Such genetic element constructs, which comprise expression cassettes for the effector but lack expression cassettes for one or more Anellovirus ORFs (e.g., Anellovirus ORF1 or a splice variant or functional fragment thereof), may be introduced into host cells. Host cells comprising such genetic element constructs may, in some instances, require additional nucleic acid constructs or integration of expression cassettes into the host cell genome for production of one or more components of the anellovector (e.g., the proteinaceous exterior proteins). In some embodiments, host cells comprising such genetic element constructs are incapable of enclosure of the genetic elements within proteinaceous exteriors in the absence of an additional nucleic construct encoding an Anellovirus ORF1 molecule. In other words, such genetic element constructs may be used for trans anellovector production methods in host cells, e.g., as described herein.

In some embodiments (e.g., cis embodiments described herein), the genetic element construct further comprises one or more expression cassettes comprising a coding sequence for one or more non-Anellovirus ORF (e.g., a non-Anellovirus Rep molecule, e.g., an AAV Rep molecule, e.g., an AAV Rep protein, e.g., an AAV Rep2 protein). Such genetic element constructs, which comprise expression cassettes for the effector as well as the one or more non-Anellovirus ORFs, may be introduced into host cells. Host cells comprising such genetic element constructs may, in some instances, be capable of producing the genetic elements and components for proteinaceous exteriors, and for enclosure of the genetic elements within proteinaceous exteriors, without requiring additional nucleic acid constructs or integration of expression cassettes into the host cell genome. In other words, such genetic element constructs may be used for cis anellovector production methods in host cells, e.g., as described herein.

In some embodiments (e.g., trans embodiments described herein), the genetic element does not comprise an expression cassette comprising a coding sequence for one or more non-Anellovirus ORFs (e.g., a non-Anellovirus Rep molecule, e.g., an AAV Rep molecule, e.g., an AAV Rep protein, e.g., an AAV Rep2 protein). Such genetic element constructs, which comprise expression cassettes for the effector but lack expression cassettes for one or more non-Anellovirus ORFs (e.g., a non-Anellovirus Rep molecule, e.g., an AAV Rep molecule, e.g., an AAV Rep protein, e.g., an AAV Rep2 protein), may be introduced into host cells. Host cells comprising such genetic element constructs may, in some instances, require additional nucleic acid constructs or integration of expression cassettes into the host cell genome for production of one or more components of the anellovector (e.g., for replication of the genetic element). In some embodiments, host cells comprising such genetic element constructs are incapable of replicating the genetic elements in the absence of an additional nucleic construct, e.g., encoding a non-Anellovirus Rep molecule, e.g., an AAV Rep molecule, e.g., an AAV Rep protein, e.g., an AAV Rep2 protein. In other words, such genetic element constructs may be used for trans anellovector production methods in host cells, e.g., as described herein.

### Exemplary Cell Types

Exemplary host cells suitable for production of anellovectors include, without limitation, mammalian cells, e.g., human cells and insect cells. In some embodiments, the host cell is a human cell or cell line. In some embodiments, the cell is an immune cell or cell line, e.g., a T cell or cell line, a cancer cell line, a hepatic cell or cell line, a neuron, a glial cell, a skin cell, an epithelial cell, a mesenchymal cell, a blood cell, an endothelial cell, a gastrointestinal cell, a progenitor cell, a precursor cell, a stem cell, a lung cell, a cardiac cell, or a muscle cell. In some embodiments, the host cell is an animal cell (e.g., a mouse cell, rat cell, rabbit cell, or hamster cell, or insect cell).

In some embodiments, the host cell is a lymphoid cell. In some embodiments, the host cell is a T cell or an immortalized T cell. In embodiments, the host cell is a Jurkat cell. In embodiments, the host cell is a MOLT cell (e.g., a MOLT-4 or a MOLT-3 cell). In embodiments, the host cell is a MOLT-4 cell.

In embodiments, the host cell is a MOLT-3 cell. In some embodiments, the host cell is an acute lymphoblastic leukemia (ALL) cell, e.g., a MOLT cell, e.g., a MOLT-4 or MOLT-3 cell. In some embodiments, the host cell is a B cell or an immortalized B cell. In some embodiments, the host cell comprises a genetic element construct (e.g., as described herein).

In some embodiments, the host cell is a MOLT cell (e.g., a MOLT-4 or a MOLT-3 cell).

In some embodiments, the host cell is an acute lymphoblastic leukemia (ALL) cell, e.g., a MOLT cell, e.g., a MOLT-4 or MOLT-3 cell.

In some embodiments, the host cell is an Expi-293 cell. In some embodiments, the host cell is an Expi-293F cell.

In an aspect, the present disclosure provides a method of manufacturing an anellovector comprising a genetic element enclosed in a proteinaceous exterior, the method comprising providing a MOLT-4 cell comprising an anellovector genetic element, and incubating the MOLT-4 cell under conditions that allow the anellovector genetic element to become enclosed in a proteinaceous exterior in the MOLT-4 cell. In some embodiments, the MOLT-4 cell further comprises one or more Anellovirus proteins (e.g., an Anellovirus ORF1 molecule) that form part or all of the proteinaceous exterior. In some embodiments, the anellovector genetic element is produced in the MOLT-4 cell, e.g., from a genetic element construct (e.g., as described herein). In some embodiments, the method further comprises introducing the anellovector genetic element construct into the MOLT-4 cell.

In an aspect, the present disclosure provides a method of manufacturing an anellovector comprising a genetic element enclosed in a proteinaceous exterior, the method comprising providing a MOLT-3 cell comprising an anellovector genetic element, and incubating the MOLT-3 cell under conditions that allow the anellovector genetic element to become enclosed in a proteinaceous exterior in the MOLT-3 cell. In some embodiments, the MOLT-3 cell further comprises one or more Anellovirus proteins (e.g., an Anellovirus ORF1 molecule) that form part or all of the proteinaceous exterior. In some embodiments, the anellovector genetic element is produced in the MOLT-3 cell, e.g., from a genetic element construct (e.g., as described herein). In some embodiments, the method further comprises introducing the anellovector genetic element construct into the MOLT-3 cell.

In some embodiments, the host cell is a human cell. In embodiments, the host cell is a HEK293T cell, HEK293F cell, A549 cell, Jurkat cell, Raji cell, Chang cell, HeLa cell Phoenix cell, MRC-5 cell, NCI-H292 cell, or Wi38 cell. In some embodiments, the host cell is a non-human primate cell (e.g., a Vero cell, CV-1 cell, or LLCMK2 cell). In some embodiments, the host cell is a murine cell (e.g., a McCoy cell). In some embodiments, the host cell is a hamster cell (e.g., a CHO cell or BHK 21 cell). In some embodiments, the host cell is a MARC-145, MDBK, RK-13, or EEL cell. In some embodiments, the host cell is an epithelial cell (e.g., a cell line of epithelial lineage).

In some embodiments, the anellovector is cultivated in continuous animal cell line (e.g., immortalized cell lines that can be serially propagated). According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST.

### Culture Conditions

Host cells comprising a genetic element and components of a proteinaceous exterior can be incubated under conditions suitable for enclosure of the genetic element within the proteinaceous exterior, thereby producing an anellovector. Suitable culture conditions include those described, e.g., in any of Examples 24, 25, 27, or 28. In some embodiments, the host cells are incubated in liquid media (e.g., Grace's Supplemented (TNM-FH), IPL-41, TC-100, Schneider's Drosophila, SF-900 II SFM, or and EXPRESS-FIVE™ SFM). In some embodiments, the host cells are incubated in adherent culture. In some embodiments, the host cells are incubated in suspension culture. In some embodiments, the host cells are incubated in a tube, bottle, microcarrier, or flask. In some embodiments, the host cells are incubated in a dish or well (e.g., a well on a plate). In some embodiments, the host cells are incubated under conditions suitable for proliferation of the host cells. In some embodiments, the host cells are incubated under conditions suitable for the host cells to release anellovectors produced therein into the surrounding supernatant.

The production of anellovector-containing cell cultures according to the present invention can be carried out in different scales (e.g., in flasks, roller bottles or bioreactors). The media used for the cultivation of the cells to be infected generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of anellovectors.

### Harvest

Anellovectors produced by host cells can be harvested, e.g., according to methods known in the art. For example, anellovectors released into the surrounding supernatant by host cells in culture can be harvested from the supernatant (e.g., as described in Example 24). In some embodiments, the supernatant is separated from the host cells to obtain the anellovectors. In some embodiments, the host cells are lysed before or during harvest. In some embodiments, the host cells are lysed in a detergent (e.g., Triton, e.g., 0.01%-0.1% Triton). In some embodiments, the anellovectors are harvested from the host cell lysates (e.g., as described in Example 10 of PCT Publication No. WO 2020/123816, incorporated by reference herein in its entirety). In some embodiments, the anellovectors are harvested from both the host cell lysates and the supernatant. In some embodiments, the purification and isolation of anellovectors is performed according to known methods in virus production, for example, as described in Rinaldi, et al., DNA Vaccines: Methods and Protocols (Methods in Molecular Biology), 3rd ed. 2014, Humana Press (incorporated herein by reference in its entirety). In some embodiments, the anellovector may be harvested and/or purified by separation of solutes based on biophysical properties, e.g., ion exchange chromatography or tangential flow filtration, prior to formulation with a pharmaceutical excipient.

### In Vitro Assembly Methods for Anellovectors

An anellovector may be produced, e.g., by in vitro assembly, e.g., in the absence of a host cell, in a cell-free suspension, or in a supernatant. In some embodiments, the genetic element is contacted to an ORF1 molecule in vitro, e.g., under conditions that allow for assembly.

In an aspect, the present disclosure provides a particle (e.g., an anellovector as described herein) produced via in vitro assembly (e.g., as described herein). The particle may, in some instances, comprise a proteinaceous exterior comprising an ORF1 molecule and a genetic element encoding an exogenous effector, which is enclosed within the proteinaceous exterior. In some embodiments, a particle produced by in vitro assembly does not include a substantial (e.g., detectable) amount of one or more constituents (e.g., small molecules, peptides, polypeptides, nucleic acids, polynucleotides, lipids, sugars, and/or organelles) from a host cell (e.g., a host cell used to produce the ORF1 molecules and/or the genetic element). In some embodiments, the particle may have one or more (e.g., 1, 2, 3, 4, 5, 6, 7, 8, or all 9) of the following characteristics:
(i) the genetic element (e.g., a DNA genetic element) does not comprise an Anellovirus 5′ UTR and/or an Anellovirus origin of replication;
(ii) the sequence encoding the exogenous effector takes up at least 90%, 95%, 96%, 97%, 98%, 99% or 100% of the genetic element (e.g., a DNA genetic element);
(iii) the heterologous nucleic acid sequence takes up at least 90%, 95%, 96%, 97%, 98%, 99% or 100% of the genetic element (e.g., a DNA genetic element);
(iv) the particle does not comprise a detectable amount of (e.g., any) polypeptides from a host cell, or comprises less than 5, 10, 15, 20, 25, 30, 40, or 50 copies of a polypeptide from a host cell;
(v) the particle does not comprise a detectable amount of (e.g., any) nucleic acid molecules from a host cell other than the genetic element (or copies thereof), or comprises less than 2, 3, 4, or 5 copies of a nucleic acid molecule from a host cell;
(vi) the particle comprises a denaturant in a concentration of less than about 0.01M, 0.1M, 0.2M, 0.3M, 0.4M, 0.5M, 0.6M, 0.7M, 0.8M, 0.9M, 1M, 1.1M, 1.2M, 1.3M, 1.5M, 1.5M, 1.6M, 1.7M, 1.8M, 1.9M, or 2M;
(vii) does not substantially replicate when introduced into a cell (e.g., a human cell); and/or
(viii) has a symmetrical morphology and/or a diameter of at least 30, 31, 32, 33, 34, or 35 nm.

In another aspect, the disclosure provides a population of the particles (e.g., the anellovectors).

In some embodiments, Anellovirus proteins to be used for in vitro assembly of a particle (e.g., an anellovector) as described herein are produced in a cell. In some embodiments, baculovirus constructs are used to produce Anellovirus proteins (e.g., one or more of an Anellovirus ORF1, ORF2, and/or ORF3 molecule, e.g., as described herein), for example, in insect cells (e.g., Sf9 cells). These proteins may then be used, e.g., for in vitro assembly to encapsidate a genetic element, e.g., a genetic element comprising RNA. In some embodiments, a polynucleotide encoding one or more Anellovirus proteins is fused to a promoter for expression in a host cell, e.g., an insect cell or an animal cell. In some embodiments, the polynucleotide is cloned into a baculovirus expression system. In some embodiments, a host cell, e.g., an insect cell, is infected with the baculovirus expression system and incubated for a period of time under conditions suitable for expression of the one or more Anellovirus proteins. In some embodiments, an infected cell is incubated for about 1, 2, 3, 4, 5, 10, 15, or 20 days. In some embodiments, an infected cell is lysed to recover the one or more Anellovirus proteins.

In some embodiments, an Anellovirus protein (e.g., an Anellovirus ORF1 molecule) is produced as described in Example 7. In some embodiments, an Anellovirus protein (e.g., an Anellovirus ORF1 molecule) is produced in insect cells as described in Example 8 or 10. In some embodiments, a plurality of Anellovirus ORF1 molecules has a propensity to self-assemble into a proteinaceous exterior, for example, to form a virus-like particle (VLP). In certain embodiments, the VLPs do not encapsulate a genetic element as described herein. In certain embodiments, the VLP comprises at least 40, 45, 50, 55, 60, 65, or 70 ORF1 molecules in its proteinaceous exterior.

In certain embodiments, a VLP comprising an Anellovirus ORF1 molecule can be denatured as described herein (e.g., using a chaotropic agent, such as urea). In embodiments, a VLP is denatured using one or more of: buffers of different pH, conditions of defined conductivity (salt content), a detergent (such as SDS (e.g., 0.1% SDS), Tween, Triton), a chaotropic agent (such as urea, e.g., as described herein), a high salt solution (e.g., a solution comprising NaCl, e.g., at a concentration of at least about 1M, e.g., at least about 0.5, 0.6, 0.7, 0.8, 0.9, 1.0, 1.1, 1.2, 1.3, 1.4, 1.5, 2, 3, 4, or 5M), or conditions involving defined temperature and time (reannealing temperatures), for example, as described in Example 12. In embodiments, a VLP is denatured using urea as described in Example 11. In embodiments, a VLP is denatured in urea at a concentration of about 1-10 M (e.g., about 1-2 M, 2-3 M, 3-4 M, 4-5 M, 5-6 M, 6-7 M, 7-8 M, 8-9 M, 9-10M, or 1-6 M). In embodiments, a VLP is denatured in urea at a concentration of about 1-10 M (e.g., about 1, 2, 3, 4, 5, 6, 7, 8, 9, or 10M). In an embodiment, a VLP is denatured in urea at a concentration of 2 M. In embodiments, a VLP is denatured under high salt conditions. In embodiments, denaturation of the VLP results in the ORF1 molecules forming capsomers (e.g., capsomeric decamers comprising about 10 copies of the ORF1 molecule).

In some embodiments, removing the capsomers from the presence of the chaotropic agent (e.g., by purifying the capsomers or removing the chaotropic agent, e.g., by dilution or dialysis) allows the ORF1 molecules to reform VLPs (e.g., VLPs comprising at least 40, 45, 50, 55, 60, 65, or 70 ORF1 molecules). In embodiments, VLPs are reformed by dialyzing out a chaotropic agent (e.g., urea) as described in Example 11. In some embodiments, VLPs are reformed in the presence of a cargo of interest, such as a genetic element (e.g., as described herein), under conditions suitable for enclosure of the cargo in the proteinaceous exterior of the VLP (e.g., as described in Example 12).In some embodiments, an isolated Anellovirus protein (e.g., an ORF1 molecule) is purified (e.g., from a cell). In some embodiments, an Anellovirus protein is purified using purification techniques including but not limited to chelating purification, heparin purification, gradient sedimentation purification, and/or SEC purification. In embodiments, an Anellovirus protein is purified as described in Example 7. In some embodiments, an Anellovirus protein (e.g., an Anellovirus ORF1 molecule) is purified from insect cells as described in Example 8 or 10. In some embodiments, a purified Anellovirus protein is mixed with a genetic element to encapsidate the genetic element, e.g., a genetic element comprising RNA. In some embodiments, a genetic element is encapisdated using an ORF1 protein, ORF2 protein, or modified version thereof. In some embodiments two nucleic acids are encapsidated. For instance, the first nucleic acid may be an mRNA e.g., chemically modified mRNA, and the second nucleic acid may be DNA.

In some embodiments, DNA encoding Anellovirus ORF1 (e.g., wildtype ORF1 protein, ORF1 proteins harboring mutations, e.g., to improve assembly efficiency, yield or stability, chimeric ORF1 protein, or fragments thereof) are expressed in insect cell lines (e.g., Sf9 and/or HighFive), animal cell lines (e.g., chicken cell lines (MDCC)), bacterial cells (e.g., E. coli) and/or mammalian cell lines (e.g., 293expi and/or MOLT4). In some embodiments, DNA encoding Anellovirus ORF1 may be untagged. In some embodiments, DNA encoding Anellovirus ORF1 may contain tags fused N-terminally and/or C-terminally. In some embodiments, DNA encoding Anellovirus ORF1 may harbor mutations, insertions or deletions within the ORF1 protein to introduce a tag, e.g., to aid in purification and/or identity determination, e.g., through immunostaining assays (including but not limited to ELISA or Western Blot). In some embodiments, DNA encoding Anellovirus ORF1 may be expressed alone or in combination with any number of helper proteins. In some embodiments, DNA encoding Anellovirus ORF1 is expressed in combination with Anellovirus ORF2 and/or ORF3 proteins.

In some embodiments, ORF1 proteins harboring mutations to improve assembly efficiency may include, but are not limited to, ORF1 proteins that harbor mutations introduced into the N-terminal Arginine Arm (ARG arm) to alter the pI of the ARG arm permitting pH sensitive nucleic acid binding to trigger particle assembly (SEQ ID 3-5). In some embodiments, ORF1 proteins harboring mutations that improve stability may include mutations to an interprotomer contacting beta strands F and G of the canonical jellyroll beta-barrel to alter hydrophobic state of the protomer surface and improve thermodynamic favorability of capsid formation.

In some embodiments, chimeric ORF1 proteins may include, but are not limited to, ORF1 proteins which have a portion or portions of their sequence replaced with comparable portions from another capsid protein, e.g., Beak and Feather Disease Virus (BFDV) capsid protein, or Hepatitis E capsid protein, e.g., ARG arm or F and G beta strands of Ring 9 ORF1 replaced with the comparable components from BFDV capsid protein. In some embodiments, chimeric ORF1 proteins may also include ORF1 proteins which have a portion or portions of their sequence replaced with comparable portions of another Anellovirus ORF1 protein (e.g., jellyroll fragments or the C-terminal portion of Ring 2 ORF1 replaced with comparable portions of Ring 9 ORF1.

In some embodiments, a genetic element to be used for in vitro assembly of a particle (e.g., an anellovector) as described herein are produced in a cell. In some embodiments, a cell is transfected with a construct (e.g., a plasmid, tandem construct, and/or an in vitro circularized nucleic acid molecule, e.g., as described herein) comprising the sequence of a genetic element. In embodiments, the cell is incubated under conditions suitable for replication of the construct. In embodiments, the cell is incubated under conditions suitable for production and/or replication of the genetic element (e.g., from the construct). In embodiments, the cell is lysed to recover the genetic element. In some embodiments, a genetic element to be used for in vitro assembly is a DNA, e.g., a single-stranded DNA (ssDNA). In embodiments, the genetic element is a negative sense ssDNA (e.g., generated as described in Example 6). In some embodiments, a genetic element to be used for in vitro assembly comprises RNA (e.g., an mRNA), for example, as described in Example 12. In some embodiments, a genetic element to be used for in vitro assembly comprises RNA (e.g., an mRNA) and the ORF1 molecule of the proteinaceous exterior comprises one or more contact residues that binds RNA (e.g., a domain from an RNA-binding protein, e.g., an mRNA-binding protein, e.g., MS2 coat protein), for example, as described in Example 12. In some embodiments, a genetic element to be used for in vitro assembly comprises RNA (e.g., an mRNA) and DNA (e.g., a DNA portion comprising a sequence that binds to an Anellovirus ORF1 molecule), for example, as described in Example 12. In certain embodiments, the RNA portion of the genetic element is covalently bound to the DNA portion of the genetic element. In certain embodiments, the RNA portion of the genetic element is hybridized to the DNA portion of the genetic element. In embodiments, the RNA portion of the genetic element comprises a region capable of hybridizing to (e.g., complementary to) at least a subsequence of the DNA portion of the genetic element.

In some embodiments, the present disclosure describes a method of making an anellovector, the method comprising: (a) providing a mixture comprising: (i) a genetic element (e.g., a genetic element comprising DNA and/or RNA), and (ii) an ORF1 molecule; and (b) incubating the mixture under conditions suitable for enclosing the genetic element within a proteinaceous exterior comprising the ORF1 molecule, thereby making an anellovector; wherein the mixture is not comprised in a cell. In some embodiments, the anellovector is assembled in vitro as described in Example 7. In some embodiments, the method further comprises, prior to the providing of (a), expressing the ORF1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule under conditions suitable for producing the ORF1 molecule. In some embodiments, the method further comprises, prior to the providing of (a), purifying the ORF1 molecule expressed by the host cell. In some embodiments, the method is performed in a cell-free system. In some embodiments, the present disclosure describes a method of manufacturing an anellovector composition, comprising: (a) providing a plurality of anellovectors or compositions according to any of the preceding embodiments; (b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and (c) formulating the plurality of anellovectors, e.g., as a pharmaceutical composition suitable for administration to a subject, e.g., if one or more of the parameters of (b) meet a specified threshold.

In Vitro Assembly Methods for anelloVLPs

An anelloVLP as described herein may be produced, e.g., by in vitro assembly, e.g., in a cell-free suspension or in a supernatant. In some embodiments, the anelloVLP is produced by contacting a plurality of ORF1 molecules (e.g., in capsomers) to an effector (e.g., an exogenous effector) in vitro, e.g., under conditions that allow for assembly. In some embodiments, the plurality of ORF1 molecules enclose the effector in a proteinaceous exterior. In some embodiments, the effector is attached to the exterior surface of a proteinaceous exterior formed from the plurality of ORF1 molecules (e.g., as a surface moiety as described herein). In some embodiments, production of an anelloVLP comprises expressing a plurality of ORF1 molecules in a cell, purifying the plurality of ORF1 molecules, denaturing virus-like particles formed by the ORF1 molecules (e.g., as described herein), and then allowing the ORF1 molecules to reform virus-like particles in the presence of an effector (e.g., an exogenous effector), thereby forming anelloVLPs comprising the ORF1 molecules enclosing the effector.

In an aspect, the present disclosure provide a particle (e.g., an anelloVLP as described herein) produced via in vitro assembly (e.g., as described herein). The particle may, in some instances, comprise a proteinaceous exterior comprising an ORF1 molecule and an effector (e.g., an exogenous effector), e.g., as described herein. In some embodiments, the effector is enclosed within the proteinaceous exterior. In some embodiments, the effector is comprised in a surface moiety (e.g., as described herein) attached to the exterior surface of the proteinaceous exterior. In some embodiments, a particle produced by in vitro assembly does not include a substantial (e.g., detectable) amount of one or more constituents (e.g., small molecules, peptides, polypeptides, nucleic acids, polynucleotides, lipids, sugars, and/or organelles) from a host cell (e.g., a host cell used to produce the ORF1 molecules and/or the genetic element). In some embodiments, the particle may have one or more (e.g., 1, 2, 3, 4, or all 5) of the following characteristics:
(i) does not comprise (e.g., does not enclose) a polynucleotide,
(ii) does not comprise (e.g., does not enclose) detectable levels of polynucleotides,
(iii) does not comprise (e.g., does not enclose) a polynucleotide of greater than 1000, 500, 200, or 100 nucleotides in length,
(iv) does not comprise (e.g., does not enclose) a polynucleotide comprising any contiguous nucleic acid sequences of at least 50, 100, 150, 200, 300, 400, 500, 600, 700, 800, 900, or 1000 nucleotides in length having least 75%, 80%, 85%, 90%, 95%, 96%, 97%, 98%, 99%, or 100% sequence identity to contiguous sequence in a wild-type Anellovirus genome (e.g., as described herein), and/or
(v) does not comprise a polynucleotide comprising an Anellovirus 5′ UTR or an origin of replication.

In another aspect, the disclosure provides a population of the particles (e.g., the anelloVLPs). In some embodiments, the population further comprises one or more anellovectors. In some embodiments, the population does not comprise anellovectors.

In some embodiments, Anellovirus proteins to be used for in vitro assembly of a particle (e.g., an anelloVLP) as described herein are produced in a cell. In some embodiments, baculovirus constructs are used to produce Anellovirus proteins (e.g., one or more of an Anellovirus ORF1, ORF2, and/or ORF3 molecule, e.g., as described herein), for example, in insect cells (e.g., Sf9 cells). These proteins may then be used, e.g., for in vitro assembly to form an anelloVLP, e.g., as described herein. In some embodiments, a polynucleotide encoding one or more Anellovirus proteins is fused to a promoter for expression in a host cell, e.g., an insect cell or an animal cell. In some embodiments, the polynucleotide is cloned into a baculovirus expression system. In some embodiments, a host cell, e.g., an insect cell, is infected with the baculovirus expression system and incubated for a period of time under conditions suitable for expression of the one or more Anellovirus proteins. In some embodiments, an infected cell is incubated for about 1, 2, 3, 4, 5, 10, 15, or 20 days. In some embodiments, an infected cell is lysed to recover the one or more Anellovirus proteins.

In some embodiments, an Anellovirus protein (e.g., an Anellovirus ORF1 molecule) is produced as described in Example 7. In some embodiments, an Anellovirus protein (e.g., an Anellovirus ORF1 molecule) is produced in insect cells as described in Example 8 or 10. In some embodiments, a plurality of Anellovirus ORF1 molecules has a propensity to self-assemble into a proteinaceous exterior, for example, to form a virus-like particle (VLP). In certain embodiments, the VLPs do not encapsulate a genetic element as described herein. In certain embodiments, the VLPs do not encapsulate an effector to be delivered to a target cell, e.g., as described herein. In certain embodiments, the VLP comprises at least 40, 45, 50, 55, 60, 65, or 70 ORF1 molecules in its proteinaceous exterior.

In certain embodiments, a VLP comprising an Anellovirus ORF1 molecule can be denatured as described herein (e.g., using a chaotropic agent, such as urea). In embodiments, a VLP is denatured using one or more of: buffers of different pH, conditions of defined conductivity (salt content), a detergent (such as SDS (e.g., 0.1% SDS), Tween, Triton), a chaotropic agent (such as urea, e.g., as described herein), a high salt solution (e.g., a solution comprising NaCl, e.g., at a concentration of at least about 1M, e.g., at least about 0.5, 0.6, 0.7, 0.8, 0.9, 1.0, 1.1, 1.2, 1.3, 1.4, 1.5, 2, 3, 4, or 5M), or conditions involving defined temperature and time (reannealing temperatures), for example, as described in Example 12. In embodiments, a VLP is denatured using urea as described in Example 11. In embodiments, a VLP is denatured in urea at a concentration of about 1-10 M (e.g., about 1-2 M, 2-3 M, 3-4 M, 4-5 M, 5-6 M, 6-7 M, 7-8 M, 8-9 M, 9-10M, or 1-6 M). In embodiments, a VLP is denatured in urea at a concentration of about 1-10 M (e.g., about 1, 2, 3, 4, 5, 6, 7, 8, 9, or 10M). In an embodiment, a VLP is denatured in urea at a concentration of 2 M. In embodiments, a VLP is denatured under high salt conditions. In embodiments, denaturation of the VLP results in the ORF1 molecules forming capsomers (e.g., capsomeric decamers comprising about 10 copies of the ORF1 molecule).

In some embodiments, removing the capsomers from the presence of the chaotropic agent (e.g., by purifying the capsomers or removing the chaotropic agent, e.g., by dilution or dialysis) allows the ORF1 molecules to reform VLPs (e.g., VLPs comprising at least 40, 45, 50, 55, 60, 65, or 70 ORF1 molecules). In embodiments, VLPs are reformed by dialyzing out a chaotropic agent (e.g., urea) as described in Example 11. In some embodiments, VLPs are reformed in the presence of a cargo of interest (e.g., an effector as described herein), for example, under conditions suitable for enclosure of the cargo in the proteinaceous exterior of the VLP (e.g., as described in Example 12). In some embodiments, an isolated Anellovirus protein (e.g., an ORF1 molecule) is purified (e.g., from a cell). In some embodiments, an Anellovirus protein is purified using purification techniques including but not limited to chelating purification, heparin purification, gradient sedimentation purification, and/or SEC purification.

In embodiments, an Anellovirus protein is purified as described in Example 7. In some embodiments, an Anellovirus protein (e.g., an Anellovirus ORF1 molecule) is purified from insect cells as described in Example 8 or 10. In some embodiments, a plurality of purified Anellovirus proteins are mixed with an effector. In embodiments, the purified Anellovirus proteins encapsulated the effector. In some embodiments, the effector is encapsulated using an ORF1 protein, ORF2 protein, or modified version thereof.

In some embodiments, DNA encoding Anellovirus ORF1 (e.g., wildtype ORF1 protein, ORF1 proteins harboring mutations, e.g., to improve assembly efficiency, yield or stability, chimeric ORF1 protein, or fragments thereof) are expressed in insect cell lines (e.g., Sf9 and/or HighFive), animal cell lines (e.g., chicken cell lines (MDCC)), bacterial cells (e.g., E. coli) and/or mammalian cell lines (e.g., 293expi and/or MOLT4). In some embodiments, DNA encoding Anellovirus ORF1 may be untagged. In some embodiments, DNA encoding Anellovirus ORF1 may contain tags fused N-terminally and/or C-terminally. In some embodiments, DNA encoding Anellovirus ORF1 may harbor mutations, insertions or deletions within the ORF1 protein to introduce a tag, e.g., to aid in purification and/or identity determination, e.g., through immunostaining assays (including but not limited to ELISA or Western Blot). In some embodiments, DNA encoding Anellovirus ORF1 may be expressed alone or in combination with any number of helper proteins. In some embodiments, DNA encoding Anellovirus ORF1 is expressed in combination with Anellovirus ORF2 and/or ORF3 proteins.

In some embodiments, ORF1 proteins harboring mutations to improve assembly efficiency may include, but are not limited to, ORF1 proteins that harbor mutations introduced into the N-terminal Arginine Arm (ARG arm) to alter the pI of the ARG arm permitting pH sensitive nucleic acid binding to trigger particle assembly (SEQ ID 3-5). In some embodiments, ORF1 proteins harboring mutations that improve stability may include mutations to an interprotomer contacting beta strands F and G of the canonical jellyroll beta-barrel to alter hydrophobic state of the protomer surface and improve thermodynamic favorability of capsid formation.

In some embodiments, chimeric ORF1 proteins may include, but are not limited to, ORF1 proteins which have a portion or portions of their sequence replaced with comparable portions from another capsid protein, e.g., Beak and Feather Disease Virus (BFDV) capsid protein, or Hepatitis E capsid protein, e.g., ARG arm or F and G beta strands of Ring 9 ORF1 replaced with the comparable components from BFDV capsid protein. In some embodiments, chimeric ORF1 proteins may also include ORF1 proteins which have a portion or portions of their sequence replaced with comparable portions of another Anellovirus ORF1 protein (e.g., jellyroll fragments or the C-terminal portion of Ring 2 ORF1 replaced with comparable portions of Ring 9 ORF1.

In some embodiments, the present disclosure describes a method of making an anelloVLP, the method comprising: (a) providing a mixture comprising: (i) an effector (e.g., an exogenous effector), and (ii) an ORF1 molecule; and (b) incubating the mixture under conditions suitable for enclosing the effector within a proteinaceous exterior comprising the ORF1 molecule, thereby making an anelloVLP; wherein the mixture is not comprised in a cell. In some embodiments, the anelloVLP is assembled in vitro as described in Example 7. In some embodiments, the method further comprises, prior to the providing of (a), expressing the ORF1 molecule, e.g., in a host cell (e.g., an insect cell or a mammalian cell). In some embodiments, the expressing comprises incubating a host cell (e.g., an insect cell or a mammalian cell) comprising a nucleic acid molecule (e.g., a baculovirus expression vector) encoding the ORF1 molecule under conditions suitable for producing the ORF1 molecule. In some embodiments, the method further comprises, prior to the providing of (a), purifying the ORF1 molecule expressed by the host cell. In some embodiments, the method is performed in a cell-free system. In some embodiments, the present disclosure describes a method of manufacturing an anelloVLP composition, comprising: (a) providing a plurality of anelloVLPs or compositions according to any of the preceding embodiments; (b) optionally evaluating the plurality for one or more of: a contaminant described herein, an optical density measurement (e.g., OD 260), particle number (e.g., by HPLC), infectivity (e.g., particle:infectious unit ratio, e.g., as determined by fluorescence and/or ELISA); and (c) formulating the plurality of anelloVLPs, e.g., as a pharmaceutical composition suitable for administration to a subject, e.g., if one or more of the parameters of (b) meet a specified threshold.

### Enrichment and Purification

Anellovectors or anelloVLPs produced as described herein can be further purified and/or enriched, e.g., to produce an anellovector preparation or an anelloVLP preparation, respectively. In some embodiments, the harvested anellovectors or anelloVLPs are isolated from other constituents or contaminants present in the harvest solution, e.g., using methods known in the art for purifying viral particles (e.g., purification by sedimentation, chromatography, and/or ultrafiltration). In some embodiments, the purification steps comprise removing one or more of serum, host cell DNA, host cell proteins, particles lacking the genetic element, and/or phenol red from the preparation. In some embodiments, the harvested anellovectors or anelloVLPs are enriched relative to other constituents or contaminants present in the harvest solution, e.g., using methods known in the art for enriching viral particles.

In some embodiments, the resultant preparation or a pharmaceutical composition comprising the preparation will be stable over an acceptable period of time and temperature, and/or be compatible with the desired route of administration and/or any devices this route of administration will require, e.g., needles or syringes.

### III. Vectors

The genetic element described herein may be included in a vector. Suitable vectors as well as methods for their manufacture and their use are well known in the prior art.

In one aspect, the invention includes a vector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid.

The genetic element or any of the sequences within the genetic element can be obtained using any suitable method. Various recombinant methods are known in the art, such as, for example screening libraries from cells harboring viral sequences, deriving the sequences from a vector known to include the same, or isolating directly from cells and tissues containing the same, using standard techniques. Alternatively or in combination, part or all of the genetic element can be produced synthetically, rather than cloned.

In some embodiments, the vector includes regulatory elements, nucleic acid sequences homologous to target genes, and various reporter constructs for causing the expression of reporter molecules within a viable cell and/or when an intracellular molecule is present within a target cell.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In some embodiments, the vector is substantially non-pathogenic and/or substantially non-integrating in a host cell or is substantially non-immunogenic in a host.

In some embodiments, the vector is in an amount sufficient to modulate one or more of phenotype, virus levels, gene expression, compete with other viruses, disease state, etc. at least about 5%, 10%, 15%, 20%, 25%, 30%, 35%, 40%, 45%, 50%, or more.

### IV. Compositions

An anellovector or or anelloVLP described herein may also be included in pharmaceutical compositions with a pharmaceutical excipient, e.g., as described herein. In some embodiments, the pharmaceutical composition comprises at least 10⁵, 106, 107, 10¹, 109, 10¹⁰, 10¹¹, 10¹², 10¹3, 10¹⁴, or 10” anellovectors or anelloVLPs. In some embodiments, the pharmaceutical composition comprises about 10⁵-10¹⁵, 10⁵-10¹⁰, or 10¹⁰-10¹⁵ anellovectors or anelloVLPs. In some embodiments, the pharmaceutical composition comprises about 10⁸(e.g., about 10⁵, 10⁶, 10⁷, 10⁸, 10⁹, or 10¹⁰) genomic equivalents/mL of the anellovector or anelloVLP. In some embodiments, the pharmaceutical composition comprises 10⁵-10¹⁰, 10⁶-10¹⁰, 10⁷-10¹⁰, 10¹⁰-10¹⁰, 10⁹-10¹⁰, 10⁵-10⁶, 10⁵-10⁷, 10⁵-10¹⁰, 10⁵-10⁹, 10⁵-10¹⁰, 10⁵-10¹², 10⁵-10¹¹, 10⁵-10¹⁴, 10⁵-10¹⁵, or 10¹⁰-10¹⁵ genomic equivalents/mL of the anellovector or anelloVLP, e.g., as determined according to the method of Example 32. In some embodiments, the pharmaceutical composition comprises sufficient anellovectors or anelloVLPs to deliver at least 1, 2, 5, or 10, 100, 500, 1000, 2000, 5000, 8,000, 1×10⁴, 1×10⁵, 1×10⁶, 1×10⁷ or greater copies of a genetic element comprised in the anellovectors or anelloVLPs per cell to a population of the eukaryotic cells. In some embodiments, the pharmaceutical composition comprises sufficient anellovectors or anelloVLPs to deliver at least about 1×10⁴, 1×10⁵, 1×10⁶, 1× or 10⁷, or about 1×10⁴-1×10⁵, 1×10⁴-1×10⁶, 1×10⁴-1×10⁷, 1×10⁵-1×10⁶, 1×10⁵-1×10⁷, or 1×10⁶-1×10⁷ copies of a genetic element comprised in the anellovectors or anelloVLPs per cell to a population of the eukaryotic cells.

In some embodiments, the pharmaceutical composition has one or more of the following characteristics: the pharmaceutical composition meets a pharmaceutical or good manufacturing practices (GMP) standard; the pharmaceutical composition was made according to good manufacturing practices (GMP); the pharmaceutical composition has a pathogen level below a predetermined reference value, e.g., is substantially free of pathogens; the pharmaceutical composition has a contaminant level below a predetermined reference value, e.g., is substantially free of contaminants; or the pharmaceutical composition has low immunogenicity or is substantially non-immunogenic, e.g., as described herein.

In some embodiments, the pharmaceutical composition comprises below a threshold amount of one or more contaminants. Exemplary contaminants that are desirably excluded or minimized in the pharmaceutical composition include, without limitation, host cell nucleic acids (e.g., host cell DNA and/or host cell RNA), animal-derived components (e.g., serum albumin or trypsin), replication-competent viruses, non-infectious particles, free viral capsid protein, adventitious agents, and aggregates.

In embodiments, the contaminant is host cell DNA. In embodiments, the composition comprises less than about 10 ng of host cell DNA per dose. In embodiments, the level of host cell DNA in the composition is reduced by filtration and/or enzymatic degradation of host cell DNA. In embodiments, the pharmaceutical composition consists of less than 10% (e.g., less than about 10%, 5%, 4%, 3%, 2%, 1%, 0.5%, or 0.1%) contaminant by weight.

In one aspect, the invention described herein includes a pharmaceutical composition comprising:
a) an anellovector comprising a genetic element comprising (i) a sequence encoding a non-pathogenic exterior protein, (ii) an exterior protein binding sequence that binds the genetic element to the non-pathogenic exterior protein, and (iii) a sequence encoding a regulatory nucleic acid; and a proteinaceous exterior that is associated with, e.g., envelops or encloses, the genetic element; and
b) a pharmaceutical excipient.

In one aspect, the invention described herein includes a pharmaceutical composition comprising:
a) an anelloVLP as described herein; and
b) a pharmaceutical excipient.

### Vesicles

In some embodiments, the composition further comprises a carrier component, e.g., a microparticle, liposome, vesicle, or exosome. In some embodiments, liposomes comprise spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are generally biocompatible, nontoxic, can deliver both hydrophilic and lipophilic drug molecules, protect their cargo from degradation by plasma enzymes, and transport their load across biological membranes (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Vesicles may comprise without limitation DOTMA, DOTAP, DOTIM, DDAB, alone or together with cholesterol to yield DOTMA and cholesterol, DOTAP and cholesterol, DOTIM and cholesterol, and DDAB and cholesterol. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

As described herein, additives may be added to vesicles to modify their structure and/or properties. For example, either cholesterol or sphingomyelin may be added to the mixture to help stabilize the structure and to prevent the leakage of the inner cargo. Further, vesicles can be prepared from hydrogenated egg phosphatidylcholine or egg phosphatidylcholine, cholesterol, and dicetyl phosphate. (see, e.g., Spuch and Navarro, Journal of Drug Delivery, vol. 2011, Article ID 469679, 12 pages, 2011. doi:10.1155/2011/469679 for review). Also, vesicles may be surface modified during or after synthesis to include reactive groups complementary to the reactive groups on the recipient cells. Such reactive groups include without limitation maleimide groups. As an example, vesicles may be synthesized to include maleimide conjugated phospholipids such as without limitation DSPE-MaL-PEG2000.

A vesicle formulation may be mainly comprised of natural phospholipids and lipids such as 1,2-distearoryl-sn-glycero-3-phosphatidyl choline (DSPC), sphingomyelin, egg phosphatidylcholines and monosialoganglioside. Formulations made up of phospholipids only are less stable in plasma. However, manipulation of the lipid membrane with cholesterol reduces rapid release of the encapsulated cargo or 1,2-dioleoyl-sn-glycero-3-phosphoethanolamine (DOPE) increases stability (see, e.g., Spuch and Navarro, Journal of Drug Delivery, vol. 2011, Article ID 469679, 12 pages, 2011. doi:10.1155/2011/469679 for review).

In embodiments, lipids may be used to form lipid microparticles. Lipids include, but are not limited to, DLin-KC2-DMA4, C₁₂₋₂₀0 and colipids disteroylphosphatidyl choline, cholesterol, and PEG-DMG may be formulated (see, e.g., Novobrantseva, Molecular Therapy-Nucleic Acids (2012) 1, e4; doi:10.1038/mtna.2011.3) using a spontaneous vesicle formation procedure. The component molar ratio may be about 50/10/38.5/1.5 (DLin-KC2-DMA or C₁₂₋₂₀0/disteroylphosphatidyl choline/cholesterol/PEG-DMG). Tekmira has a portfolio of approximately 95 patent families, in the U.S. and abroad, that are directed to various aspects of lipid microparticles and lipid microparticles formulations (see, e.g., U.S. Pat. Nos. 7,982,027; 7,799,565; 8,058,069; 8,283,333; 7,901,708; 7,745,651; 7,803,397; 8,101,741; 8,188,263; 7,915,399; 8,236,943 and 7,838,658 and European Pat. Nos. 1766035; 1519714; 1781593 and 1664316), all of which may be used and/or adapted to the present invention.

In some embodiments, microparticles comprise one or more solidified polymer(s) that is arranged in a random manner. The microparticles may be biodegradable. Biodegradable microparticles may be synthesized, e.g., using methods known in the art including without limitation solvent evaporation, hot melt microencapsulation, solvent removal, and spray drying. Exemplary methods for synthesizing microparticles are described by Bershteyn et al., Soft Matter 4:1787-1787, 2008 and in US 2008/0014144 A1, the specific teachings of which relating to microparticle synthesis are incorporated herein by reference.

Exemplary synthetic polymers which can be used to form biodegradable microparticles include without limitation aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as albumin, alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In general, these materials degrade either by enzymatic hydrolysis or exposure to water, by surface or bulk erosion.

The microparticles' diameter ranges from 0.1-1000 micrometers (μm). In some embodiments, their diameter ranges in size from 1-750 μm, or from 50-500 μm, or from 100-250 μm. In some embodiments, their diameter ranges in size from 50-1000 μm, from 50-750 μm, from 50-500 μm, or from 50-250 μm. In some embodiments, their diameter ranges in size from 0.05-1000 μm, from 10-1000 μm, from 100-1000 μm, or from 500-1000 μm. In some embodiments, their diameter is about 0.5 μm, about 10 μm, about 50 μm, about 100 μm, about 200 μm, about 300 μm, about 350 μm, about 400 μm, about 450 μm, about 500 μm, about 550 μm, about 600 μm, about 650 μm, about 700 μm, about 750 μm, about 800 μm, about 850 μm, about 900 μm, about 950 μm, or about 1000 μm. As used in the context of microparticle diameters, the term “about” means+/−5% of the absolute value stated.

In some embodiments, a ligand is conjugated to the surface of the microparticle via a functional chemical group (carboxylic acids, aldehydes, amines, sulfhydryls and hydroxyls) present on the surface of the particle and present on the ligand to be attached. Functionality may be introduced into the microparticles by, for example, during the emulsion preparation of microparticles, incorporation of stabilizers with functional chemical groups.

Another example of introducing functional groups to the microparticle is during post-particle preparation, by direct crosslinking particles and ligands with homo- or heterobifunctional crosslinkers. This procedure may use a suitable chemistry and a class of crosslinkers (CDI, EDAC, glutaraldehydes, etc. as discussed in more detail below) or any other crosslinker that couples ligands to the particle surface via chemical modification of the particle surface after preparation. This also includes a process whereby amphiphilic molecules such as fatty acids, lipids or functional stabilizers may be passively adsorbed and adhered to the particle surface, thereby introducing functional end groups for tethering to ligands.

In some embodiments, the microparticles may be synthesized to comprise one or more targeting groups on their exterior surface to target a specific cell or tissue type (e.g., cardiomyocytes). These targeting groups include without limitation receptors, ligands, antibodies, and the like. These targeting groups bind their partner on the cells' surface. In some embodiments, the microparticles will integrate into a lipid bilayer that comprises the cell surface and the mitochondria are delivered to the cell.

The microparticles may also comprise a lipid bilayer on their outermost surface. This bilayer may be comprised of one or more lipids of the same or different type. Examples include without limitation phospholipids such as phosphocholines and phosphoinositols. Specific examples include without limitation DMPC, DOPC, DSPC, and various other lipids such as those described herein for liposomes.

In some embodiments, the carrier comprises nanoparticles, e.g., as described herein.

In some embodiments, the vesicles or microparticles described herein are functionalized with a diagnostic agent. Examples of diagnostic agents include, but are not limited to, commercially available imaging agents used in positron emissions tomography (PET), computer assisted tomography (CAT), single photon emission computerized tomography, x-ray, fluoroscopy, and magnetic resonance imaging (MRI); and contrast agents. Examples of suitable materials for use as contrast agents in MRI include gadolinium chelates, as well as iron, magnesium, manganese, copper, and chromium.

### Carriers

A composition (e.g., pharmaceutical composition) described herein may comprise, be formulated with, and/or be delivered in, a carrier. In one aspect, the invention includes a composition, e.g., a pharmaceutical composition, comprising a carrier (e.g., a vesicle, a liposome, a lipid nanoparticle, an exosome, a red blood cell, an exosome (e.g., a mammalian or plant exosome), a fusosome) comprising (e.g., encapsulating) a composition described herein (e.g., an anellovector, anelloVLP, Anellovirus, or genetic element described herein).

In some embodiments, the compositions and systems described herein can be formulated in liposomes or other similar vesicles. Generally, liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes generally have one or more (e.g., all) of the following characteristics: biocompatibility, nontoxicity, can deliver both hydrophilic and lipophilic drug molecules, can protect their cargo from degradation by plasma enzymes, and can transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679; and Zylberberg & Matosevic. 2016. Drug Delivery, 23:9, 3319-3329, doi: 10.1080/10717544.2016.1177136).

Vesicles can be made from several different types of lipids; however, phospholipids are most commonly used to generate liposomes as drug carriers. Methods for preparation of multilamellar vesicle lipids are known (see, for example, U.S. Pat. No. 6,693,086, the teachings of which relating to multilamellar vesicle lipid preparation are incorporated herein by reference). Although vesicle formation can be spontaneous when a lipid film is mixed with an aqueeous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by, e.g., extruding through filters of decreasing size, as described in Templeton et al., Nature Biotech, 15:647-652, 1997.

Lipid nanoparticles (LNPs) are another example of a carrier that provides a biocompatible and biodegradable delivery system for the pharmaceutical compositions described herein. See, e.g., Gordillo-Galeano et al. European Journal of Pharmaceutics and Biopharmaceutics. Volume 133, December 2018, Pages 285-308. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; doi.org/10.1016/j.apsb.2016.02.001.

Ex vivo differentiated red blood cells can also be used as a carrier for a composition described herein. See, e.g., WO2015073587; WO2017123646; WO2017123644; WO2018102740; WO2016183482; WO2015153102; WO2018151829; WO2018009838; Shi et al. 2014. Proc Natl Acad Sci USA. 111(28): 10131-10136; U.S. Pat. No. 9,644,180; Huang et al. 2017. Nature Communications 8: 423; Shi et al. 2014. Proc Natl Acad Sci USA. 111(28): 10131-10136.

Fusosome compositions, e.g., as described in WO2018208728, can also be used as carriers to deliver a composition described herein.

### Membrane Penetrating Polypeptides

In some embodiments, the composition further comprises a membrane penetrating polypeptide (MPP) to carry the components into cells or across a membrane, e.g., cell or nuclear membrane.

Membrane penetrating polypeptides that are capable of facilitating transport of substances across a membrane include, but are not limited to, cell-penetrating peptides (CPPs)(see, e.g., U.S. Pat. No. 8,603,966), fusion peptides for plant intracellular delivery (see, e.g., Ng et al., PLoS One, 2016, 11:e0154081), protein transduction domains, Trojan peptides, and membrane translocation signals (MTS) (see, e.g., Tung et al., Advanced Drug Delivery Reviews 55:281-294 (2003)). Some MPP are rich in amino acids, such as arginine, with positively charged side chains.

Membrane penetrating polypeptides have the ability of inducing membrane penetration of a component and allow macromolecular translocation within cells of multiple tissues in vivo upon systemic administration. A membrane penetrating polypeptide may also refer to a peptide which, when brought into contact with a cell under appropriate conditions, passes from the external environment in the intracellular environment, including the cytoplasm, organelles such as mitochondria, or the nucleus of the cell, in amounts significantly greater than would be reached with passive diffusion.

Components transported across a membrane may be reversibly or irreversibly linked to the membrane penetrating polypeptide. A linker may be a chemical bond, e.g., one or more covalent bonds or non-covalent bonds. In some embodiments, the linker is a peptide linker. Such a linker may be between 2-30 amino acids, or longer. The linker includes flexible, rigid or cleavable linkers.

### Combinations

In one aspect, the anellovector, anelloVLP, or composition described herein may also include one or more heterologous moieties. In one aspect, the anellovector or composition comprising an anellovector or anelloVLP described herein may also include one or more heterologous moiety in a fusion. In some embodiments, a heterologous moiety may be linked with the genetic element. In some embodiments, a heterologous moiety may be enclosed in the proteinaceous exterior as part of the anellovector or anelloVLP. In some embodiments, a heterologous moiety may be administered with the anellovector or anelloVLP.

In one aspect, the invention includes a cell or tissue comprising any one of the anellovectors or anelloVLP and heterologous moieties described herein.

In another aspect, the invention includes a pharmaceutical composition comprising an anellovector or anelloVLP and the heterologous moiety described herein.

In some embodiments, the heterologous moiety may be a virus (e.g., an effector (e.g., a drug, small molecule), a targeting agent (e.g., a DNA targeting agent, antibody, receptor ligand), a tag (e.g., fluorophore, light sensitive agent such as KillerRed), or an editing or targeting moiety described herein.

In some embodiments, a membrane translocating polypeptide described herein is linked to one or more heterologous moieties. In one embodiment, the heterologous moiety is a small molecule (e.g., a peptidomimetic or a small organic molecule with a molecular weight of less than 2000 daltons), a peptide or polypeptide (e.g., an antibody or antigen-binding fragment thereof), a nanoparticle, an aptamer, or pharmacoagent.

### Viruses

In some embodiments, the composition may further comprise a virus as a heterologous moiety, e.g., a single stranded DNA virus, e.g., Anellovirus, Bidnavirus, Circovirus, Geminivirus, Genomovirus, Inovirus, Microvirus, Nanovirus, Parvovirus, and Spiravirus. In some embodiments, the composition may further comprise a double stranded DNA virus, e.g., Adenovirus, Ampullavirus, Ascovirus, Asfarvirus, Baculovirus, Fusellovirus, Globulovirus, Guttavirus, Hytrosavirus, Herpesvirus, Iridovirus, Lipothrixvirus, Nimavirus, and Poxvirus. In some embodiments, the composition may further comprise an RNA virus, e.g., Alphavirus, Furovirus, Hepatitis virus, Hordeivirus, Tobamovirus, Tobravirus, Tricornavirus, Rubivirus, Birnavirus, Cystovirus, Partitivirus, and Reovirus. In some embodiments, the anellovector or anelloVLP is administered with a virus as a heterologous moiety.

In some embodiments, the heterologous moiety may comprise a non-pathogenic, e.g., symbiotic, commensal, native, virus. In some embodiments, the non-pathogenic virus is one or more anelloviruses, e.g., Alphatorquevirus (TT), Betatorquevirus (TTM), and Gammatorquevirus (TTMD). In some embodiments, the anellovirus may include a Torque Teno Virus (TT), a SEN virus, a Sentinel virus, a TTV-like mini virus, a TT virus, a TT virus genotype 6, a TT virus group, a TTV-like virus DXL1, a TTV-like virus DXL2, a Torque Teno-like Mini Virus (TTM), or a Torque Teno-like Midi Virus (TTMD). In some embodiments, the non-pathogenic virus comprises one or more sequences having at least at least about 60%, 70% 80%, 85%, 90% 95%, 96%, 97%, 98% and 99% nucleotide sequence identity to any one of the nucleotide sequences described herein, e.g., as listed in any one of Tables N1-N25.

In some embodiments, the heterologous moiety may comprise one or more viruses that are identified as lacking in the subject. For example, a subject identified as having dyvirosis may be administered a composition comprising an anellovector and one or more viral components or viruses that are imbalanced in the subject or having a ratio that differs from a reference value, e.g., a healthy subject.

In some embodiments, the heterologous moiety may comprise one or more non-anelloviruses, e.g., adenovirus, herpes virus, pox virus, vaccinia virus, SV40, papilloma virus, an RNA virus such as a retrovirus, e.g., lenti virus, a single-stranded RNA virus, e.g., hepatitis virus, or a double-stranded RNA virus e.g., rotavirus. In some embodiments, the anellovector or the virus is defective, or requires assistance in order to produce infectious particles. Such assistance can be provided, e.g., by using helper cell lines that contain a nucleic acid, e.g., plasmids or DNA integrated into the genome, encoding one or more of (e.g., all of) the structural genes of the replication defective anellovector or virus under the control of regulatory sequences within the LTR. Suitable cell lines for replicating the anellovectors described herein include cell lines known in the art, e.g., A549 cells, which can be modified as described herein.

### Targeting Moiety

In some embodiments, the composition, anellovector, or anelloVLP described herein may further comprise a targeting moiety, e.g., a targeting moiety that specifically binds to a molecule of interest present on a target cell. The targeting moiety may modulate a specific function of the molecule of interest or cell, modulate a specific molecule (e.g., enzyme, protein or nucleic acid), e.g., a specific molecule downstream of the molecule of interest in a pathway, or specifically bind to a target to localize the anellovector, or anelloVLP, or genetic element. For example, a targeting moiety may include a therapeutic that interacts with a specific molecule of interest to increase, decrease or otherwise modulate its function.

### Tagging or Monitoring Moiety

In some embodiments, the composition, anellovector, or anelloVLP described herein may further comprise a tag to label or monitor the anellovector, anelloVLP, or genetic element described herein. The tagging or monitoring moiety may be removable by chemical agents or enzymatic cleavage, such as proteolysis or intein splicing. An affinity tag may be useful to purify the tagged polypeptide using an affinity technique. Some examples include, chitin binding protein (CBP), maltose binding protein (MBP), glutathione-S-transferase (GST), and poly(His) tag. A solubilization tag may be useful to aid recombinant proteins expressed in chaperone-deficient species such as E. coli to assist in the proper folding in proteins and keep them from precipitating. Some examples include thioredoxin (TRX) and poly(NANP). The tagging or monitoring moiety may include a light sensitive tag, e.g., fluorescence. Fluorescent tags are useful for visualization. GFP and its variants are some examples commonly used as fluorescent tags. Protein tags may allow specific enzymatic modifications (such as biotinylation by biotin ligase) or chemical modifications (such as reaction with FlAsH-EDT2 for fluorescence imaging) to occur. Often tagging or monitoring moiety are combined, in order to connect proteins to multiple other components. The tagging or monitoring moiety may also be removed by specific proteolysis or enzymatic cleavage (e.g. by TEV protease, Thrombin, Factor Xa or Enteropeptidase).

### Nanoparticles

In some embodiments, the composition, anellovector, or anelloVLP described herein may further comprise a nanoparticle. Nanoparticles include inorganic materials with a size between about 1 and about 1000 nanometers, between about 1 and about 500 nanometers in size, between about 1 and about 100 nm, between about 50 nm and about 300 nm, between about 75 nm and about 200 nm, between about 100 nm and about 200 nm, and any range therebetween. Nanoparticles generally have a composite structure of nanoscale dimensions. In some embodiments, nanoparticles are typically spherical although different morphologies are possible depending on the nanoparticle composition. The portion of the nanoparticle contacting an environment external to the nanoparticle is generally identified as the surface of the nanoparticle. In nanoparticles described herein, the size limitation can be restricted to two dimensions and so that nanoparticles include composite structure having a diameter from about 1 to about 1000 nm, where the specific diameter depends on the nanoparticle composition and on the intended use of the nanoparticle according to the experimental design. For example, nanoparticles used in therapeutic applications typically have a size of about 200 nm or below.

Additional desirable properties of the nanoparticle, such as surface charges and steric stabilization, can also vary in view of the specific application of interest. Exemplary properties that can be desirable in clinical applications such as cancer treatment are described in Davis et al, Nature 2008 vol. 7, pages 771-782; Duncan, Nature 2006 vol. 6, pages 688-701; and Allen, Nature 2002 vol. 2 pages 750-763, each incorporated herein by reference in its entirety. Additional properties are identifiable by a skilled person upon reading of the present disclosure. Nanoparticle dimensions and properties can be detected by techniques known in the art. Exemplary techniques to detect particles dimensions include but are not limited to dynamic light scattering (DLS) and a variety of microscopies such at transmission electron microscopy (TEM) and atomic force microscopy (AFM). Exemplary techniques to detect particle morphology include but are not limited to TEM and AFM. Exemplary techniques to detect surface charges of the nanoparticle include but are not limited to zeta potential method. Additional techniques suitable to detect other chemical properties comprise by ¹H, “B, and ¹³C and ¹⁹F NMR, UV/Vis and infrared/Raman spectroscopies and fluorescence spectroscopy (when nanoparticle is used in combination with fluorescent labels) and additional techniques identifiable by a skilled person.

### Small Molecules

In some embodiments, the composition, anellovector, or anelloVLP described herein may further comprise a small molecule. Small molecule moieties include, but are not limited to, small peptides, peptidomimetics (e.g., peptoids), amino acids, amino acid analogs, synthetic polynucleotides, polynucleotide analogs, nucleotides, nucleotide analogs, organic and inorganic compounds (including heterorganic and organomettallic compounds) generally having a molecular weight less than about 5,000 grams per mole, e.g., organic or inorganic compounds having a molecular weight less than about 2,000 grams per mole, e.g., organic or inorganic compounds having a molecular weight less than about 1,000 grams per mole, e.g., organic or inorganic compounds having a molecular weight less than about 500 grams per mole, and salts, esters, and other pharmaceutically acceptable forms of such compounds. Small molecules may include, but are not limited to, a neurotransmitter, a hormone, a drug, a toxin, a viral or microbial particle, a synthetic molecule, and agonists or antagonists.

Examples of suitable small molecules include those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference. Some examples of small molecules include, but are not limited to, prion drugs such as tacrolimus, ubiquitin ligase or HECT ligase inhibitors such as heclin, histone modifying drugs such as sodium butyrate, enzymatic inhibitors such as 5-aza-cytidine, anthracyclines such as doxorubicin, beta-lactams such as penicillin, anti-bacterials, chemotherapy agents, anti-virals, modulators from other organisms such as VP64, and drugs with insufficient bioavailability such as chemotherapeutics with deficient pharmacokinetics.

In some embodiments, the small molecule is an epigenetic modifying agent, for example such as those described in de Groote et al. Nuc. Acids Res. (2012):1-18. Exemplary small molecule epigenetic modifying agents are described, e.g., in Lu et al. J. Biomolecular Screening 17.5(2012):555-71, e.g., at Table 1 or 2, incorporated herein by reference. In some embodiments, an epigenetic modifying agent comprises vorinostat or romidepsin. In some embodiments, an epigenetic modifying agent comprises an inhibitor of class I, II, III, and/or IV histone deacetylase (HDAC). In some embodiments, an epigenetic modifying agent comprises an activator of SirTI. In some embodiments, an epigenetic modifying agent comprises Garcinol, Lys-CoA, C646, (+)-JQI, I-BET, BICI, MS120, DZNep, UNC0321, EPZ004777, AZ505, AMI-I, pyrazole amide 7b, benzo[d]imidazole 17b, acylated dapsone derivative (e.e.g, PRMTI), methylstat, 4,4′-dicarboxy-2,2′-bipyridine, SID 85736331, hydroxamate analog 8, tanylcypromie, bisguanidine and biguanide polyamine analogs, UNC669, Vidaza, decitabine, sodium phenyl butyrate (SDB), lipoic acid (LA), quercetin, valproic acid, hydralazine, bactrim, green tea extract (e.g., epigallocatechin gallate (EGCG)), curcumin, sulforphane and/or allicin/diallyl disulfide. In some embodiments, an epigenetic modifying agent inhibits DNA methylation, e.g., is an inhibitor of DNA methyltransferase (e.g., is 5-azacitidine and/or decitabine). In some embodiments, an epigenetic modifying agent modifies histone modification, e.g., histone acetylation, histone methylation, histone sumoylation, and/or histone phosphorylation. In some embodiments, the epigenetic modifying agent is an inhibitor of a histone deacetylase (e.g., is vorinostat and/or trichostatin A).

In some embodiments, the small molecule is a pharmaceutically active agent. In one embodiment, the small molecule is an inhibitor of a metabolic activity or component. Useful classes of pharmaceutically active agents include, but are not limited to, antibiotics, anti-inflammatory drugs, angiogenic or vasoactive agents, growth factors and chemotherapeutic (anti-neoplastic) agents (e.g., tumour suppressers). One or a combination of molecules from the categories and examples described herein or from (Orme-Johnson 2007, Methods Cell Biol. 2007; 80:813-26) can be used. In one embodiment, the invention includes a composition comprising an antibiotic, anti-inflammatory drug, angiogenic or vasoactive agent, growth factor or chemotherapeutic agent.

### Peptides or Proteins

In some embodiments, the composition, anellovector, or anelloVLP described herein may further comprise a peptide or protein. The peptide moieties may include, but are not limited to, a peptide ligand or antibody fragment (e.g., antibody fragment that binds a receptor such as an extracellular receptor), neuropeptide, hormone peptide, peptide drug, toxic peptide, viral or microbial peptide, synthetic peptide, and agonist or antagonist peptide.

Peptides moieties may be linear or branched. The peptide has a length from about 5 to about 200 amino acids, about 15 to about 150 amino acids, about 20 to about 125 amino acids, about 25 to about 100 amino acids, or any range therebetween.

Some examples of peptides include, but are not limited to, fluorescent tags or markers, antigens, antibodies, antibody fragments such as single domain antibodies, ligands and receptors such as glucagon-like peptide-1 (GLP-1), GLP-2 receptor 2, cholecystokinin B (CCKB) and somatostatin receptor, peptide therapeutics such as those that bind to specific cell surface receptors such as G protein-coupled receptors (GPCRs) or ion channels, synthetic or analog peptides from naturally-bioactive peptides, anti-microbial peptides, pore-forming peptides, tumor targeting or cytotoxic peptides, and degradation or self-destruction peptides such as an apoptosis-inducing peptide signal or photosensitizer peptide.

Peptides useful in the invention described herein also include small antigen-binding peptides, e.g., antigen binding antibody or antibody-like fragments, such as single chain antibodies, nanobodies (see, e.g., Steeland et al. 2016. Nanobodies as therapeutics: big opportunities for small antibodies. Drug Discov Today: 21(7):1076-113). Such small antigen binding peptides may bind a cytosolic antigen, a nuclear antigen, an intra-organellar antigen.

In some embodiments, the composition, anellovector, or anelloVLP described herein includes a polypeptide linked to a ligand that is capable of targeting a specific location, tissue, or cell.

### Oligonucleotide Aptamers

In some embodiments, the composition, anellovector, or anelloVLP described herein may further comprise an oligonucleotide aptamer. Aptamer moieties are oligonucleotide or peptide aptamers. Oligonucleotide aptamers are single-stranded DNA or RNA (ssDNA or ssRNA) molecules that can bind to pre-selected targets including proteins and peptides with high affinity and specificity.

Oligonucleotide aptamers are nucleic acid species that may be engineered through repeated rounds of in vitro selection or equivalently, SELEX (systematic evolution of ligands by exponential enrichment) to bind to various molecular targets such as small molecules, proteins, nucleic acids, and even cells, tissues and organisms. Aptamers provide discriminate molecular recognition, and can be produced by chemical synthesis. In addition, aptamers may possess desirable storage properties, and elicit little or no immunogenicity in therapeutic applications.

Both DNA and RNA aptamers can show robust binding affinities for various targets. For example, DNA and RNA aptamers have been selected for t lysozyme, thrombin, human immunodeficiency virus trans-acting responsive element (HIV TAR), (see en.wikipedia.org/wiki/Aptamer cite_note-10), hemin, interferon γ, prostate specific antigen (PSA), dopamine, and the non-classical oncogene, heat shock factor 1 (HSF1).

### Peptide Aptamers

In some embodiments, the composition, anellovector, or anelloVLP described herein may further comprise a peptide aptamer. Peptide aptamers have one (or more) short variable peptide domains, including peptides having low molecular weight, 12-14 kDa. Peptide aptamers may be designed to specifically bind to and interfere with protein-protein interactions inside cells.

Peptide aptamers are artificial proteins selected or engineered to bind specific target molecules. These proteins include of one or more peptide loops of variable sequence. They are typically isolated from combinatorial libraries and often subsequently improved by directed mutation or rounds of variable region mutagenesis and selection. In vivo, peptide aptamers can bind cellular protein targets and exert biological effects, including interference with the normal protein interactions of their targeted molecules with other proteins. In particular, a variable peptide aptamer loop attached to a transcription factor binding domain is screened against the target protein attached to a transcription factor activating domain. In vivo binding of the peptide aptamer to its target via this selection strategy is detected as expression of a downstream yeast marker gene. Such experiments identify particular proteins bound by the aptamers, and protein interactions that the aptamers disrupt, to cause the phenotype. In addition, peptide aptamers derivatized with appropriate functional moieties can cause specific post-translational modification of their target proteins, or change the subcellular localization of the targets Peptide aptamers can also recognize targets in vitro. They have found use in lieu of antibodies in biosensors and used to detect active isoforms of proteins from populations containing both inactive and active protein forms. Derivatives known as tadpoles, in which peptide aptamer “heads” are covalently linked to unique sequence double-stranded DNA “tails”, allow quantification of scarce target molecules in mixtures by PCR (using, for example, the quantitative real-time polymerase chain reaction) of their DNA tails.

Peptide aptamer selection can be made using different systems, but the most used is currently the yeast two-hybrid system. Peptide aptamers can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

### V. Host Cells

The invention is further directed to a host or host cell comprising an anellovector or anelloVLP as described herein. In some embodiments, the host or host cell is a plant, insect, bacteria, fungus, vertebrate, mammal (e.g., human), or other organism or cell. In certain embodiments, as confirmed herein, provided anellovectors infect a range of different host cells. Target host cells include cells of mesodermal, endodermal, or ectodermal origin. Target host cells include, e.g., epithelial cells, muscle cells, white blood cells (e.g., lymphocytes), kidney tissue cells, lung tissue cells.

In some embodiments, the anellovector or anelloVLP is substantially non-immunogenic in the host. In certain embodiments, the anellovector or genetic element thereof, or the anelloVLP, fails to produce an undesired substantial response by the host's immune system. Some immune responses include, but are not limited to, humoral immune responses (e.g., production of antigen-specific antibodies) and cell-mediated immune responses (e.g., lymphocyte proliferation).

In some embodiments, a host or a host cell is contacted with (e.g., infected with) an anellovector or anelloVLP. In some embodiments, the host is a mammal, such as a human. The amount of the anellovector or anelloVLP in the host can be measured at any time after administration. In certain embodiments, a time course of anellovector growth in a culture is determined.

In some embodiments, the anellovector, e.g., an anellovector as described herein, is heritable. In some embodiments, the anellovector is transmitted linearly in fluids and/or cells from mother to child. In some embodiments, daughter cells from an original host cell comprise the anellovector. In some embodiments, a mother transmits the anellovector to child with an efficiency of at least 25%, 50%, 60%, 70%, 80%, 85%, 90%, 95%, or 99%, or a transmission efficiency from host cell to daughter cell at least 25%, 50%, 60%, 70%, 80%, 85%, 90%, 95%, or 99%. In some embodiments, the anellovector in a host cell has a transmission efficiency during meiosis of at 25%, 50%, 60%, 70%, 80%, 85%, 90%, 95%, or 99%. In some embodiments, the anellovector in a host cell has a transmission efficiency during mitosis of at least 25%, 50%, 60%, 70%, 80%, 85%, 90%, 95%, or 99%. In some embodiments, the anellovector in a cell has a transmission efficiency between about 10%-20%, 20%-30%, 30%-40%, 40%-50%, 50%-60%, 60%-70%, 70%-75%, 75%-80%, 80%-85%, 85%-90%, 90%-95%, 95%-99%, or any percentage therebetween.

In some embodiments, the anellovector, e.g., anellovector replicates within the host cell. In one embodiment, the anellovector is capable of replicating in a mammalian cell, e.g., human cell. In other embodiments, the anellovector is replication deficient or replication incompetent.

While in some embodiments the anellovector replicates in the host cell, the anellovector does not integrate into the genome of the host, e.g., with the host's chromosomes. In some embodiments, the anellovector has a negligible recombination frequency, e.g., with the host's chromosomes. In some embodiments, the anellovector has a recombination frequency, e.g., less than about 1.0 cM/Mb, 0.9 cM/Mb, 0.8 cM/Mb, 0.7 cM/Mb, 0.6 cM/Mb, 0.5 cM/Mb, 0.4 cM/Mb, 0.3 cM/Mb, 0.2 cM/Mb, 0.1 cM/Mb, or less, e.g., with the host's chromosomes.

### VI. Methods of Use

The anellovectors and compositions comprising anellovectors or anelloVLPs described herein may be used in methods of treating a disease, disorder, or condition, e.g., in a subject (e.g., a mammalian subject, e.g., a human subject) in need thereof. Administration of a pharmaceutical composition described herein may be, for example, by way of parenteral (including intravenous, intratumoral, intraperitoneal, intramuscular, intracavity, and subcutaneous) administration. The anellovectors or anelloVLP may be administered alone or formulated as a pharmaceutical composition.

The anellovectors or anelloVLP may be administered in the form of a unit-dose composition, such as a unit dose parenteral composition. Such compositions are generally prepared by admixture and can be suitably adapted for parenteral administration. Such compositions may be, for example, in the form of injectable and infusable solutions or suspensions or suppositories or aerosols.

In some embodiments, administration of a anellovector or composition comprising same, e.g., as described herein, may result in delivery of a genetic element comprised by the anellovector to a target cell, e.g., in a subject.

An anellovector or anelloVLP, or composition thereof, described herein, e.g., comprising an effector (e.g., an endogenous or exogenous effector), may be used to deliver the effector to a cell, tissue, or subject. In some embodiments, the anellovector, or anelloVLP, or composition thereof is used to deliver the effector to bone marrow, blood, heart, GI or skin. Delivery of an effector by administration of an anellovector composition or anelloVLP composition described herein may modulate (e.g., increase or decrease) expression levels of a noncoding RNA or polypeptide in the cell, tissue, or subject. Modulation of expression level in this fashion may result in alteration of a functional activity in the cell to which the effector is delivered. In some embodiments, the modulated functional activity may be enzymatic, structural, or regulatory in nature.

In some embodiments, the anellovector or anelloVLP, or copies thereof, are detectable in a cell 24 hours (e.g., 1 day, 2 days, 3 days, 4 days, 5 days, 6 days, 1 week, 2 weeks, 3 weeks, 4 weeks, 30 days, or 1 month) after delivery into a cell. In embodiments, an anellovector, anelloVLP, or composition thereof mediates an effect on a target cell, and the effect lasts for at least 1, 2, 3, 4, 5, 6, or 7 days, 2, 3, or 4 weeks, or 1, 2, 3, 6, or 12 months. In some embodiments (e.g., wherein the anellovector or composition thereof comprises a genetic element encoding an exogenous protein), the effect lasts for less than 1, 2, 3, 4, 5, 6, or 7 days, 2, 3, or 4 weeks, or 1, 2, 3, 6, or 12 months.

Examples of diseases, disorders, and conditions that can be treated with an anellovector or anelloVLP described herein, or a composition comprising the anellovector or anelloVLP, include, without limitation: immune disorders, interferonopathies (e.g., Type I interferonopathies), infectious diseases, inflammatory disorders, autoimmune conditions, cancer (e.g., a solid tumor, e.g., lung cancer, non-small cell lung cancer, e.g., a tumor that expresses a gene responsive to mIR-625, e.g., caspase-3), and gastrointestinal disorders. In some embodiments, the anellovector or anelloVLP modulates (e.g., increases or decreases) an activity or function in a cell with which the anellovector is contacted. In some embodiments, the anellovector or anelloVLP modulates (e.g., increases or decreases) the level or activity of a molecule (e.g., a nucleic acid or a protein) in a cell with which the anellovector or anelloVLP is contacted. In some embodiments, the anellovector or anelloVLP decreases viability of a cell, e.g., a cancer cell, with which the anellovector or anelloVLP is contacted, e.g., by at least about 10%, 20%, 30%, 40%, 50%, 60%, 70%, 80%, 90%, 95%, 99%, or more. In some embodiments, the anellovector or anelloVLP comprises an effector, e.g., an miRNA, e.g., miR-625, that decreases viability of a cell, e.g., a cancer cell, with which the anellovector or anelloVLP is contacted, e.g., by at least about 10%, 20%, 30%, 40%, 50%, 60%, 70%, 80%, 90%, 95%, 99%, or more. In some embodiments, the anellovector or anelloVLP increases apoptosis of a cell, e.g., a cancer cell, e.g., by increasing caspase-3 activity, with which the anellovector is contacted, e.g., by at least about 10%, 20%, 30%, 40%, 50%, 60%, 70%, 80%, 90%, 95%, 99%, or more. In some embodiments, the anellovector or anelloVLP comprises an effector, e.g., an miRNA, e.g., miR-625, that increases apoptosis of a cell, e.g., a cancer cell, e.g., by increasing caspase-3 activity, with which the anellovector or anelloVLP is contacted, e.g., by at least about 10%, 20%, 30%, 40%, 50%, 60%, 70%, 80%, 90%, 95%, 99%, or more.

### VII. Methods of Production

### Producing the Genetic Element

Methods of making the genetic element of the anellovector are described in, for example, Khudyakov & Fields, Artificial DNA: Methods and Applications, CRC Press (2002); in Zhao, Synthetic Biology: Tools and Applications, (First Edition), Academic Press (2013); and Egli & Herdewijn, Chemistry and Biology of Artificial Nucleic Acids, (First Edition), Wiley-VCH (2012).

In some embodiments, the genetic element may be designed using computer-aided design tools. The anellovector may be divided into smaller overlapping pieces (e.g., in the range of about 100 bp to about 10 kb segments or individual ORFs) that are easier to synthesize. These DNA segments are synthesized from a set of overlapping single-stranded oligonucleotides. The resulting overlapping synthons are then assembled into larger pieces of DNA, e.g., the anellovector. The segments or ORFs may be assembled into the anellovector, e.g., in vitro recombination or unique restriction sites at 5′ and 3′ ends to enable ligation.

The genetic element can alternatively be synthesized with a design algorithm that parses the anellovector into oligo-length fragments, creating optimal design conditions for synthesis that take into account the complexity of the sequence space. Oligos are then chemically synthesized on semiconductor-based, high-density chips, where over 200,000 individual oligos are synthesized per chip. The oligos are assembled with an assembly techniques, such as BioFab®, to build longer DNA segments from the smaller oligos. This is done in a parallel fashion, so hundreds to thousands of synthetic DNA segments are built at one time.

Each genetic element or segment of the genetic element may be sequence verified. In some embodiments, high-throughput sequencing of RNA or DNA can take place using AnyDot.chips (Genovoxx, Germany), which allows for the monitoring of biological processes (e.g., miRNA expression or allele variability (SNP detection). In particular, the AnyDot-chips allow for 10×-50× enhancement of nucleotide fluorescence signal detection. AnyDot.chips and methods for using them are described in part in International Publication Application Nos. WO 02088382, WO 03020968, WO 0303 1947, WO 2005044836, PCTEP 05105657, PCMEP 05105655; and German Patent Application Nos. DE 101 49 786, DE 102 14 395, DE 103 56 837, DE 10 2004 009 704, DE 10 2004 025 696, DE 10 2004 025 746, DE 10 2004 025 694, DE 10 2004 025 695, DE 10 2004 025 744, DE 10 2004 025 745, and DE 10 2005 012 301.

Other high-throughput sequencing systems include those disclosed in Venter, J., et al. Science 16 Feb. 2001; Adams, M. et al, Science 24 Mar. 2000; and M. J, Levene, et al. Science 299:682-686, January 2003; as well as US Publication Application No. 20030044781 and 2006/0078937. Overall such systems involve sequencing a target nucleic acid molecule having a plurality of bases by the temporal addition of bases via a polymerization reaction that is measured on a molecule of nucleic acid, i.e., the activity of a nucleic acid polymerizing enzyme on the template nucleic acid molecule to be sequenced is followed in real time. The sequence can then be deduced by identifying which base is being incorporated into the growing complementary strand of the target nucleic acid by the catalytic activity of the nucleic acid polymerizing enzyme at each step in the sequence of base additions. A polymerase on the target nucleic acid molecule complex is provided in a position suitable to move along the target nucleic acid molecule and extend the oligonucleotide primer at an active site. A plurality of labeled types of nucleotide analogs are provided proximate to the active site, with each distinguishably type of nucleotide analog being complementary to a different nucleotide in the target nucleic acid sequence. The growing nucleic acid strand is extended by using the polymerase to add a nucleotide analog to the nucleic acid strand at the active site, where the nucleotide analog being added is complementary to the nucleotide of the target nucleic acid at the active site. The nucleotide analog added to the oligonucleotide primer as a result of the polymerizing step is identified. The steps of providing labeled nucleotide analogs, polymerizing the growing nucleic acid strand, and identifying the added nucleotide analog are repeated so that the nucleic acid strand is further extended and the sequence of the target nucleic acid is determined.

In some embodiments, shotgun sequencing is performed. In shotgun sequencing, DNA is broken up randomly into numerous small segments, which are sequenced using the chain termination method to obtain reads. Multiple overlapping reads for the target DNA are obtained by performing several rounds of this fragmentation and sequencing. Computer programs then use the overlapping ends of different reads to assemble them into a continuous sequence.

In some embodiments, factors for replicating or packaging may be supplied in cis or in trans, relative to the genetic element. For example, when supplied in cis, the genetic element may comprise one or more genes encoding an Anellovirus ORF1, ORF1/1, ORF1/2, ORF2, ORF2/2, ORF2/3, or ORF2t/3, e.g., as described herein. In some embodiments, replication and/or packaging signals can be incorporated into a genetic element, for example, to induce amplification and/or encapsulation. In some embodiments, this is done both in context of larger regions of the anellovector genome (e.g., inserting effectors into a specific site in the genome, or replacing viral ORFs with effectors).

In another example, when supplied in trans, the genetic element may lack genes encoding one or more of an Anellovirus ORF1, ORF1/1, ORF1/2, ORF2, ORF2/2, ORF2/3, or ORF2t/3, e.g., as described herein; this protein or proteins may be supplied, e.g., by another nucleic acid, e.g., a helper nucleic acid.

In some embodiments, minimal cis signals (e.g., 5′ UTR and/or GC-rich region) are present in the genetic element. In some embodiments, the genetic element does not encode replication or packaging factors (e.g., replicase and/or capsid proteins). Such factors may, in some embodiments, be supplied by one or more helper nucleic acids (e.g., a helper viral nucleic acid, a helper plasmid, or a helper nucleic acid integrated into the host cell genome). In some embodiments, the helper nucleic acids express proteins and/or RNAs sufficient to induce amplification and/or packaging, but may lack their own packaging signals. In some embodiments, the genetic element and the helper nucleic acid are introduced into the host cell (e.g., concurrently or separately), resulting in amplification and/or packaging of the genetic element but not of the helper nucleic acid.

### In Vitro Circularization

In some instances, the genetic element to be packaged into a proteinaceous exterior is a single stranded circular DNA. The genetic element may, in some instances, be introduced into a host cell in a form other than a single stranded circular DNA. For example, the genetic element may be introduced into the host cell as a double-stranded circular DNA. The double-stranded circular DNA may then be converted into a single-stranded circular DNA in the host cell (e.g., a host cell comprising a suitable enzyme for rolling circle replication, e.g., an Anellovirus Rep protein, e.g., Rep68/78, Rep60, RepA, RepB, Pre, MobM, TraX, TrwC, Mob02281, Mob02282, NikB, ORF50240, NikK, TecH, OrfJ, or Tral, e.g., as described in Wawrzyniak et al. 2017, Front. Microbiol. 8: 2353; incorporated herein by reference with respect to the listed enzymes). In some embodiments, the double-stranded circular DNA is produced by in vitro circularization, e.g., as described in Example 35 of PCT Publication No. WO 2020/123816, incorporated by reference herein in its entirety. Generally, in vitro circularized DNA constructs can be produced by digesting a plasmid comprising the sequence of a genetic element to be packaged, such that the genetic element sequence is excised as a linear DNA molecule. The resultant linear DNA can then be ligated, e.g., using a DNA ligase, to form a double-stranded circular DNA. In some instances, a double-stranded circular DNA produced by in vitro circularization can undergo rolling circle replication, e.g., as described herein. Without wishing to be bound by theory, it is contemplated that in vitro circularization results in a double-stranded DNA construct that can undergo rolling circle replication without further modification, thereby being capable of producing single-stranded circular DNA of a suitable size to be packaged into an anellovector, e.g., as described herein. In some embodiments, the double-stranded DNA construct is smaller than a plasmid (e.g., a bacterial plasmid). In some embodiments, the double-stranded DNA construct is excised from a plasmid (e.g., a bacterial plasmid) and then circularized, e.g., by in vitro circularization.

### Producing the Anellovector

The genetic elements and vectors comprising the genetic elements prepared as described herein can be used in a variety of ways to express the anellovector in appropriate host cells. In some embodiments, the genetic element and vectors comprising the genetic element are transfected in appropriate host cells and the resulting RNA may direct the expression of the anellovector gene products, e.g., non-pathogenic protein and protein binding sequence, at high levels. Host cell systems which provide for high levels of expression include continuous cell lines that supply viral functions, such as cell lines superinfected with APV or MPV, respectively, cell lines engineered to complement APV or MPV functions, etc.

In some embodiments, the anellovector is produced as described in any of Examples 22, 25, or 26. In some embodiments, the anellovector is produced as described in any of Examples 1, 2, 5, 6, or 15-17 of PCT Publication No. WO 2020/123816, incorporated by reference herein in its entirety.

In some embodiments, the anellovector is cultivated in continuous animal cell lines in vitro. According to one embodiment of the invention, the cell lines may include porcine cell lines. The cell lines envisaged in the context of the present invention include immortalised porcine cell lines such as, but not limited to the porcine kidney epithelial cell lines PK-15 and SK, the monomyeloid cell line 3D4/31 and the testicular cell line ST. Also, other mammalian cells lines are included, such as CHO cells (Chinese hamster ovaries), MARC-145, MDBK, RK-13, EEL. Additionally or alternatively, particular embodiments of the methods of the invention make use of an animal cell line which is an epithelial cell line, i.e. a cell line of cells of epithelial lineage. Cell lines susceptible to infection with anellovectors include, but are not limited to cell lines of human or primate origin, such as human or primate kidney carcinoma cell lines.

In some embodiments, the genetic elements and vectors comprising the genetic elements are transfected into cell lines that express a viral polymerase protein in order to achieve expression of the anellovector. To this end, transformed cell lines that express an anellovector polymerase protein may be utilized as appropriate host cells. Host cells may be similarly engineered to provide other viral functions or additional functions.

To prepare the anellovector disclosed herein, a genetic element or vector comprising the genetic element disclosed herein may be used to transfect cells which provide anellovector proteins and functions required for replication and production. Alternatively, cells may be transfected with helper virus before, during, or after transfection by the genetic element or vector comprising the genetic element disclosed herein. In some embodiments, a helper virus may be useful to complement production of an incomplete viral particle. The helper virus may have a conditional growth defect, such as host range restriction or temperature sensitivity, which allows the subsequent selection of transfectant viruses. In some embodiments, a helper virus may provide one or more replication proteins utilized by the host cells to achieve expression of the anellovector. In some embodiments, the host cells may be transfected with vectors encoding viral proteins such as the one or more replication proteins. In some embodiments, a helper virus comprises an antiviral sensitivity.

The genetic element or vector comprising the genetic element disclosed herein can be replicated and produced into anellovector particles by any number of techniques known in the art, as described, e.g., in U.S. Pat. Nos. 4,650,764; 5,166,057; 5,854,037; European Patent Publication EP 0702085A1; U.S. patent application Ser. No. 09/152,845; International Patent Publications PCT WO97/12032; WO96/34625; European Patent Publication EP-A780475; WO 99/02657; WO 98/53078; WO 98/02530; WO 99/15672; WO 98/13501; WO 97/06270; and EPO 780 47SA1, each of which is incorporated by reference herein in its entirety.

The production of anellovector-containing cell cultures according to the present invention can be carried out in different scales, such as in flasks, roller bottles or bioreactors. The media used for the cultivation of the cells to be infected are known to the skilled person and can generally comprise the standard nutrients required for cell viability, but may also comprise additional nutrients dependent on the cell type. Optionally, the medium can be protein-free and/or serum-free. Depending on the cell type the cells can be cultured in suspension or on a substrate. In some embodiments, different media is used for growth of the host cells and for production of anellovectors.

The purification and isolation of anellovectors can be performed according to methods known by the skilled person in virus production and is described for example by Rinaldi, et al., DNA Vaccines: Methods and Protocols (Methods in Molecular Biology), 3rd ed. 2014, Humana Press.

In one aspect, the present invention includes a method for the in vitro replication and propagation of the anellovector as described herein, which may comprise the following steps: (a) transfecting a linearized genetic element into a cell line sensitive to anellovector infection; (b) harvesting the cells and isolating cells showing the presence of the genetic element; (c) culturing the cells obtained in step (b) for at least three days, such as at least one week or longer, depending on experimental conditions and gene expression; and (d) harvesting the cells of step (c).

In some embodiments, an anellovector may be introduced to a host cell line grown to a high cell density. In some embodiments, the anellovector may be harvested and/or purified by separation of solutes based on biophysical properties, e.g., ion exchange chromatography or tangential flow filtration, prior to formulation with a pharmaceutical excipient.

### VIII. Administration/Delivery

The composition (e.g., a pharmaceutical composition comprising an anellovector or anelloVLP as described herein) may be formulated to include a pharmaceutically acceptable excipient. Pharmaceutical compositions may optionally comprise one or more additional active substances, e.g. therapeutically and/or prophylactically active substances. Pharmaceutical compositions of the present invention may be sterile and/or pyrogen-free. General considerations in the formulation and/or manufacture of pharmaceutical agents may be found, for example, in Remington: The Science and Practice of Pharmacy 21st ed., Lippincott Williams & Wilkins, 2005 (incorporated herein by reference).

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product.

In one aspect, the invention features a method of delivering an anellovector or anelloVLP to a subject. The method includes administering a pharmaceutical composition comprising an anellovector or anelloVLP as described herein to the subject. In some embodiments, the administered anellovector or anelloVLP replicates in the subject (e.g., becomes a part of the virome of the subject).

The pharmaceutical composition may include wild-type or native viral elements and/or modified viral elements. The anellovector may include one or more of the sequences (e.g., nucleic acid sequences or nucleic acid sequences encoding amino acid sequences thereof) in any one of Tables N1-N25 or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any one of Tables N1-N25. The anellovector may comprise a nucleic acid molecule comprising a nucleic acid sequence with at least about 60%, 65%, 70%, 75%, 80%, 85%, 90% 95%, 96%, 97%, 98% and 99% sequence identity to one or more of the sequences in any one of Tables N1-N25. The anellovector may comprise a nucleic acid molecule encoding an amino acid sequence with at least about 60%, 65%, 70%, 75%, 80%, 85%, 90% 95%, 96%, 97%, 98% and 99% sequence identity to any one of the amino acid sequences in any one of Tables A1-A25. The anellovector may comprise a polypeptide comprising an amino acid sequence with at least about 60%, 65%, 70%, 75%, 80%, 85%, 90% 95%, 96%, 97%, 98% and 99% sequence identity to any one of the amino acid sequences in any one of Tables A1-A25. The anellovector may include one or more of the sequences in any one of Tables A1-A25 or N1-N25, or a sequence with at least about 60%, 65%, 70%, 75%, 80%, 85%, 90% 95%, 96%, 97%, 98% and 99% nucleotide sequence identity to any one of the nucleotide sequences or a sequence that is complementary to the sequence in any one of Tables N1-N25.

In some embodiments, the anellovector or anelloVLP is sufficient to increase (stimulate) endogenous gene and protein expression, e.g., at least about 5%, 10%, 15%, 20%, 25%, 30%, 35%, 40%, 45%, 50%, or more as compared to a reference, e.g., a healthy control. In certain embodiments, the anellovector or anelloVLP is sufficient to decrease (inhibit) endogenous gene and protein expression, e.g., at least about 5%, 10%, 15%, 20%, 25%, 30%, 35%, 40%, 45%, 50%, or more as compared to a reference, e.g., a healthy control.

In some embodiments, the anellovector or anelloVLP inhibits/enhances one or more viral properties, e.g., tropism, infectivity, immunosuppression/activation, in a host or host cell, e.g., at least about 5%, 10%, 15%, 20%, 25%, 30%, 35%, 40%, 45%, 50%, or more as compared to a reference, e.g., a healthy control.

In some embodiments, the subject is administered the pharmaceutical composition further comprising one or more viral strains that are not represented in the viral genetic information.

In some embodiments, the pharmaceutical composition comprising an anellovector or anelloVLP described herein is administered in a dose and time sufficient to modulate a viral infection. Some non-limiting examples of viral infections include adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, Human enterovirus 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16, Human papillomavirus 18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, O′nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika Virus. In certain embodiments, the anellovector or anelloVLP is sufficient to outcompete and/or displace a virus already present in the subject, e.g., at least about 5%, 10%, 15%, 20%, 25%, 30%, 35%, 40%, 45%, 50%, or more as compared to a reference. In certain embodiments, the anellovector or anelloVLP is sufficient to compete with chronic or acute viral infection. In certain embodiments, the anellovector or anelloVLP may be administered prophylactically to protect from viral infections (e.g. a provirotic). In some embodiments, the anellovector or anelloVLP is in an amount sufficient to modulate (e.g., phenotype, virus levels, gene expression, compete with other viruses, disease state, etc. at least about 5%, 10%, 15%, 20%, 25%, 30%, 35%, 40%, 45%, 50%, or more).

### Redosing

The anellovectors or anelloVLPs described herein can, in some instances, be used as a delivery vehicle that can be administered in multiple doses (e.g., doses administered separately). While not wishing to be bound by theory, in some embodiments, an anellovector or anelloVLP (e.g., as described herein) induces a relatively low immune response (as measured, for example, as 50% GMT values, e.g., as observed in Example 29), e.g., allowing for repeated dosing of a subject with one or more anellovectors or anelloVLPs (e.g., multiple doses of the same anellovector or anelloVLP or different anellovectors or anelloVLPs). In an aspect, the invention provides a method of delivering an effector, comprising administering to a subject a first plurality of anellovectors or anelloVLPs and then a second plurality of anellovectors or anelloVLPs. In some embodiments, the second plurality of anellovectors or anelloVLPs comprise the same proteinaceous exterior as the anellovectors or anelloVLPs of the first plurality. In another aspect, the invention provides a method of selecting a subject (e.g., a human subject) to receive an effector, wherein the subject previously received, or was identified as having received, a first plurality of anellovectors or anelloVLPs comprising a genetic element encoding an effector, in which the method involves selecting the subject to receive a second plurality of anellovectors or anelloVLPs comprising a genetic element encoding an effector (e.g., the same effector as that encoded by the genetic element of the first plurality of anellovectors or anelloVLPs, or a different effector as that encoded by the genetic element of the first plurality of anellovectors or anelloVLPs). In another aspect, the invention provides a method of identifying a subject (e.g., a human subject) as suitable to receive a second plurality of anellovectors or anelloVLPs, the method comprising identifying the subject has having previously received a first plurality of anellovectors or anelloVLPs comprising a genetic element encoding an effector, wherein the subject being identified as having received the first plurality of anellovectors or anelloVLPs is indicative that the subject is suitable to receive the second plurality of anellovectors or anelloVLPs.

In some embodiments, the second plurality of anellovectors or anelloVLPs comprises a proteinaceous exterior with at least one surface epitope in common with the anellovectors or anelloVLPs of the first plurality of anellovectors or anelloVLPs. In some embodiments, the anellovectors or anelloVLPs of the first plurality and the anellovectors or anelloVLPs of the second plurality carry genetic elements encoding the same effector. In some embodiments, the anellovectors or anelloVLPs of the first plurality and the anellovectors or anelloVLPs of the second plurality carry genetic elements encoding different effectors.

In some embodiments, the second plurality comprises about the same quantity and/or concentration of anellovectors or anelloVLPs as the first plurality (e.g., when normalized to the body mass of the subject at the time of administration), e.g., the second plurality comprises 90-110%, e.g., 95-10⁵% of the number of anellovectors or anelloVLPs in the first plurality when normalized to body mass of the subject at the time of administration. In some embodiments, wherein the first plurality comprises a greater dosage of anellovectors or anelloVLPs than the second plurality, e.g., wherein the first plurality comprises a greater quantity and/or concentration of anellovectors or anelloVLPs relative to the second plurality. In some embodiments, wherein the first plurality comprises a lower dosage of anellovectors or anelloVLPs than the second plurality, e.g., wherein the first plurality comprises a lower quantity and/or concentration of anellovectors or anelloVLPs relative to the second plurality.

In some embodiments, the subject is evaluated between the administration of the first and second pluralities of anellovectors or anelloVLPs, e.g., for the presence (e.g., persistence) of anellovectors or anelloVLPs from the first plurality, or progeny thereof. In some embodiments, the subject is administered the second plurality of anellovectors or anelloVLPs if the presence of anellovectors or anelloVLPs from the first plurality, or the progeny thereof, are not detected.

In some embodiments, the second plurality is administered to the subject at least 1, 2, 3, or 4 weeks, or 1, 2, 3, 4, 5, 6, 7, 8, 9, 10, 11, or 12 months, or 1, 2, 3, 4, 5, 10, or 20 years after the administration of the first plurality to the subject. In some embodiments, the second plurality is administered to the subject between 1-2 weeks, 2-3 weeks, 3-4 weeks, 1-2 months, 3-4 months, 4-5 months, 5-6 months, 6-7 months, 7-8 months, 8-9 months, 9-10 months, 10-11 months, 11-12 months, 1-2 years, 2-3 years, 3-4 years, 4-5 years, 5-10 years, or 10-20 years after the administration of the first plurality to the subject. In some embodiments, the method comprises administering a repeated dose of anellovectors or anelloVLPs over the course of at least 1, 2, 3, 4, or 5 years.

In some embodiments, the method further comprises assessing, after administration of the first plurality and before administration of the second plurality, one or more of:
a) the level or activity of the effector in the subject (e.g., by detecting a protein effector, e.g., by ELISA; by detecting a nucleic acid effector, e.g., by RT-PCR, or by detecting a downstream effect of the effector, e.g., level of an endogenous gene affected by the effector);
b) the level or activity of the anellovector or anelloVLP of the first plurality in the subject (e.g., by detecting the level of the VP1 of the anellovector or anelloVLP);
c) the presence, severity, progression, or a sign or symptom of a disease in the subject that the anellovector or anelloVLP was administered to treat; and/or
d) the presence or level of an immune response, e.g., neutralizing antibodies, against an anellovector or anelloVLP.

In some embodiments, the method further comprises administering to the subject a third, fourth, fifth, and/or further plurality of anellovectors or anelloVLPs, e.g., as described herein.

In some embodiments, the first plurality and the second plurality are administered via the same route of administration, e.g., intravenous administration. In some embodiments, the first plurality and the second plurality are administered via different routes of administration. In some embodiments, the first and the second pluralities are administered by the same entity (e.g., the same health care provider). In some embodiments, the first and the second pluralities are administered by different entities (e.g., different health care providers).

All references and publications cited herein are hereby incorporated by reference.

The following examples are provided to further illustrate some embodiments of the present invention, but are not intended to limit the scope of the invention; it will be understood by their exemplary nature that other procedures, methodologies, or techniques known to those skilled in the art may alternatively be used. Additional examples can be found, for example, in PCT Publication No. WO 2020/123816, incorporated by reference herein in its entirety.

### EXAMPLES

### Table of Contents

Example 1: Expression of ORF1 constructs for conjugation
Example 2: Conjugation of peptides via NHS click chemistry
Example 3: Conjugation of polypeptides via two-step click chemistry
Example 4: Conjugation of polypeptides via maleimide conjugation
Example 5: Genetic grafting of surface effector to the VLP surface
Example 6: Generation of circular ssDNA transgene for in vitro assembly of Anellovectors
Example 7: Anellovector ORF1 VLP expression in insect or mammalian cells and purification
Example 8: Expression of Ring2 and Ring10 Anellovirus ORF1s in insect cells
Example 9: Expression of CAV capsid protein VP1 in mammalian cells
Example 10: Expression of Ring2 ORF1 and ORF2 in insect cells
Example 11: Disassembly of Ring2 VLPs using urea
Example 12: Dissociation of Anellovirus VLPs and reassembly around nucleic acid cargoes
Example 13: Exemplary sequence s for ORF1 molecules
Example 14: Production of Anellovirus proteins in a baculovirus expression system
Example 15: Expression of Ring1 ORFs in Sf9 cells
Example 16: Expression of Ring2 ORFs in Sf9 cells
Example 17: Expression of all Ring2 ORFs simultaneously in Sf9 cells
Example 18: Co-delivery and independent expression of anellovirus genomes and recombinant Anellovirus ORFs in Sf9 cells
Example 19: Anellovirus ORF1 associates with DNA in Sf9 cells to form complexes isolated by isopycnic centrifugation
Example 20: Expression of ORF1 protein from a diverse array of Anelloviruses using baculovirus
Example 21: In vitro assembly of anellovectors using components produced via baculovirus system
Example 22: Identification and use of protein binding sequences: putative protein-binding sites in the

### Anellovirus Genome

Example 23: Replication-deficient anellovectors and helper viruses
Example 24: Manufacturing process for replication-competent anellovectors
Example 25: Manufacturing process of replication-deficient anellovectors: recovery and scaling up of production of replication-deficient anellovectors
Example 26: Production of anellovectors using suspension cells: production of anellovectors in cells in suspension.
Example 27: Quantification of anellovector genome equivalents by qPCR: development of a hydrolysis probe-based quantitative PCR assay to quantify anellovectors
Example 28: Tandem copies of the Anellovirus genome
Example 29: In vitro circularized Anellovirus genomes: constructs comprising circular, double stranded Anelloviral genome DNA with minimal non-viral DNA
Example 30: Production of anellovectors containing chimeric ORF1 with hypervariable domains from different Torque Teno Virus strains
Example 31: Design of an anellovector harboring a DNA payload
Example 32: In vitro circularized genome as input material for producing anellovectors in vitro
Example 33: Antibody generation and western blot analysis
Example 34: Construct design, cell culture and protein expression/purification
Example 35: Negative-stained EM data collection and analysis
Example 36: Cryo-EM data collection and data analysis and molecular refinement
Example 37: Circular dichroism spectroscopy
Example 38: Anellovirus particle structure
Example 39: Anellovirus jelly roll domains
Example 40: Anellovirus spike domains
Example 41: Evasion of immune system
Example 42: Production and purification of AnelloVLPs
Example 43: Conjugation of AnelloVLPs
Example 44: VLP conjugation to SARS-CoV-2 RBD peptide

### Example 1. Expression of ORF1 Constructs for Conjugation

In nature, Anelloviruses encapsidate circularized negative sense (ns) single stranded (ss) DNA genomes in part through interactions between the genome and the N-terminal arginine rich motif (ARM). UV absorption evidence suggests recombinant ORF1 containing the ARM forms virus like particles (VLPs) that are bound to nucleic acids, presumably non-specific host cell DNA fragments. To remove these potentially undesired host cell impurities, ORF1 constructs are generated in which the ARM is deleted.

Anellovirus ORF1 proteins, such as an ORF1 protein in which the ARM has been deleted, harbor a basic residue (lysine) on the particle surface which would be suitable for conjugating surface effectors including peptides, proteins such as antibodies, glycan groups or neuclic acid entities which are covalently bound to N-hydroxysuccinimide (NHS). NHS is a click-chemistry which binds to amino groups such as those located on lysine residues.

### Example 2. Conjugation of Peptides Via NHS Click Chemistry

Based on the structural analysis described herein, basic residues (lysine residues) of Ring 10 ORF1 were identified as surface exposed on the VLP viral surface. Several surface effectors can be conjugated to the amine groups of lysine by adding the click-chemistry entity NHS. In one example, the short antigenic region of the malaria CS protein (for example, the NANP repeat region known to be immunogenic in the R21 malaria vaccine currently being evaluated in the clinic) can be synthesized with an NHS moiety. By mixing the NANP-NHS peptide with ORF1 VLPs, we can conjugate the antigenic region of the R21 vaccine candidate to the ORF1 VLP generating a novel malaria particle as a vaccine candidate. Conjugation of other entities to the surface lysines though NHS conjugation could also be generated such as anti-sense oligomers (ASOs) or glycans from other bacteria known to provide an immune response in vaccines (such as, for example, the glycans of Prevnar13).

### Example 3. Conjugation of Polypeptides Via Two-Step Click Chemistry

As discussed in Example 2, surface exposed lysines are available for NHS conjugation. However, not every surface effector may be available for synthetic addition of NHS such as peptides, oligomers or glycans. In one example, larger surface effectors such as proteins (antibody fragments or larger vaccine antigens) could be generated with free cysteine, which can further be conjugated with click-chemistries, for example, by virtue of maleimide-click-chemistry linkers (maleimide being a click chemistry that binds thiols of free cysteine), which in turn can be added to paired chemistries on conjugated to free lysine on the VLP surface. In one example, a free cysteine could be engineered into a polypeptide encoding the C-terminal (immunogenic) portion of the malaria CS protein. This protein could then be conjugated by virtue of its free cysteine to a click chemistry moiety such as azide. The ORF1 VLP could be further conjugated by NHS-DBCO (DBCO being the click-chemistry partner of azide) by virtue of the surface lysines on ORF1. When the VLP-DBCO species is combined with the Azide-Malaria polypeptide species the DBCO-Azide conjugation would produce a covalently attached Malaria-VLP particle suitable as a malaria vaccine candidate. This two-step conjugation approach could be suitable for other larger surface effectors where direct synthesis with click-chemistry moieties is not available, including antibodies and larger nucleic acid oligomers.

### Example 4. Conjugation of Polypeptides Via Maleimide Conjugation

In Examples 2 or 3 above, conjugation of surface effectors is done through conjugation of NHS moieties to surface lysines. This approach could have the added advantage of adding several surface effectors to the ORF1, depending on how many lysines are surface exposed in a given strain of ORF1. However, specific conjugation to ORF1 may be desired for some surface effectors to generate a more controlled product. In this example, a Ring 10 ORF1 structure (determined according to the structural analysis described herein) is used to define surface exposed regions of the VLP and introduce a free cysteine suitable for maleimide conjugation (maleimide being a click-chemistry that binds thiols in free cysteine residues), for example, at residues in the P2 domain of Ring10, such as Threonine 365. These engineered VLPs, perhaps in combination with deletion of the ARM as described in Example 1, would provide a cleaner, more controlled conjugation. By replacing the NHS species in Examples 2 and 3 with maleimide moieties, the conjugation of the surface effector, such as a maleimide malaria peptide, would be specifically targeted to a particular residue of interest, e.g., at a controlled stoichiometry.

To ensure only the desired target cysteines are available for modification with antigenic peptides or other surface effector molecules, the native cysteine residues of ORF1 (e.g., located at positions: 57, 64, 112, 131, 220, 223, 626) will also be replaced with either serine or alanine residues. When the residue to be mutated is on the surface of the molecule and the sidechain is exposed to solvent, replacing a cysteine with a serine is a conservative alteration as the two residues are isosteric and therefore should not disrupt the 3-dimensional structure of the protein. When the residue to be mutated is buried inside of the structure, alanine is a more conservative alteration as the polar side chain of serine is likely to destabilize the structure.

### Example 5. Genetic Grafting of Surface Effector to the VLP Surface

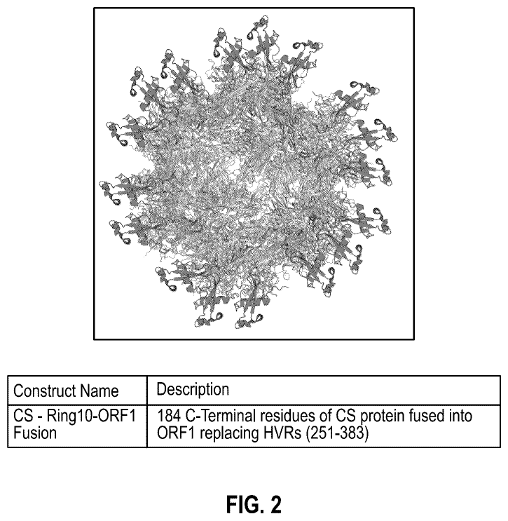

Using a structure determined for Ring 10, the region of the VLP that is surface exposed can be identified, for example, such as an HVR region. In this example, a VLP construct is generated in which a surface effector coding sequence replaces or is inserted into the HVR region of ORF. One example could be the genetic fusion of the malaria CS immunogenic portion into the HVR region of ORF1 VLPs (FIG. 2). In these genetic fusions, the VLP does not require conjugation, but rather a fusion protein comprises an ORF1 portion and a surface effector portion, such that the surface effector portion is displayed on the outer surface of the VLP. The advantage of this approach would be alleviating the need to conjugate the VLP post purification, which may not be an efficient process. Another advantage would be that each copy of the ORF1 molecule would be known to contain one copy of the surface effector. In some iterations it may be advantageous to express ORF1 with and without surface effectors generating mixed VLPs with fewer surface effectors.

In an example, a surface effector and ORF1 fusion protein comprises an amino acid sequence as listed in Table E1 below.

TABLE E1
Exemplary ORF1-surface effector fusion protein amino acid sequences

| Construct Name | Amino Acid Sequence |
|---|---|
| R21 construct | ANPNANPNANPNANPNANPNANPNANPNANPNANPNANPNANPNANPN |
| [184 C-Terminal residues of CS | ANPNANPNANPNKNNQGNGQGHNMPNDPNRNVDENANANSAVKNNNNE |
| protein (CS-Cterm) fused | EPSDKHIKEYLNKIQNSLSTEWSPCSVTCGNGIQVRIKPGSANKPKDE |
| to the Hepatitis B Virus Surface | LDYANDIEKKICKMEKCSSVFNVVNSSIGLIMVLSFLFLNENTTSGFL |
| Antigen (HBsAg)] | GPLLVLQAGFFLLTRILTIPQSLDSWWTSLNFQGGAPTCPGQNSQSPT |
| | SNHSPTSCPPICPGYRWMCLRRFIIFLFILLLCLIFLLVLLDYQGMLP |
| | VCPLLPGTSTTGTGPCRTCTIPAQGTSMFPSCCCTKPSDGNCTCIPIP |
| | SSWAFARFLWEWASVRFSWLSLLVPFVQWFAGLSPTVWLSVIWMMWYR |
| | GPSLYNTLSPFLPLLPISFCLWVYI |
| R21 construct + C-Terminal | ANPNANPNANPNANPNANPNANPNANPNANPNANPNANPNANPNANPN |
| Cysteine | ANPNANPNANPNKNNQGNGQGHNMPNDPNRNVDENANANSAVKNNNNE |
| [184 C-Terminal residues of CS | EPSDKHIKEYLNKIQNSLSTEWSPCSVTCGNGIQVRIKPGSANKPKDE |
| protein (CS-Cterm-184) fused | LDYANDIEKKICKMEKCSSVFNVVNSSIGLIMVLSFLFLNENTTSGFL |
| to the Hepatitis B Virus Surface | GPLLVLQAGFFLLTRILTIPQSLDSWWTSLNFQGGAPTCPGQNSQSPT |
| Antigen (HBsAg) | SNHSPTSCPPICPGYRWMCLRRFIIFLFILLLCLIFLLVLLDYQGMLP |
| with a Cysteine residue for | VCPLLPGTSTTGTGPCRTCTIPAQGTSMFPSCCCTKPSDGNCTCIPIP |
| conjugation] | SSWAFARFLWEWASVRFSWLSLLVPFVQWFAGLSPTVWLSVIWMMWYR |
| | GPSLYNTLSPFLPLLPISFCLWVYIC |
| SB-7062 R21 CS C-Terminus | MKIKTGARILALSALTTMMESASALAANPNANPNANPNANPNANPNAN |
| [184 C-Terminal residues of CS | PNANPNANPNANPNANPNANPNANPNANPNANPNANPNKNNQGNGQGH |
| protein | NMPNDPNRNVDENANANSAVKNNNNEEPSDKHIKEYLNKIQNSLSTEW |
| for 293 expression] | SPCSVTCGNGIQVRIKPGSANKPKDELDYANDIEKKICKMEKCSSVFN |
| | VVNSSIGLIMVLSFLFLNPHHHHHHA |
| SB-7062 R21 CS C-Terminus + | MKIKTGARILALSALTTMMFSASALAANPNANPNANPNANPNANPNAN |
| C-Terminal Cysteine | PNANPNANPNANPNANPNANPNANPNANPNANPNANPNKNNQGNGQGH |
| [184 C-Terminal residues of CS | NMPNDPNRNVDENANANSAVKNNNNEEPSDKHIKEYLNKIQNSLSTEW |
| protein | SPCSVTCGNGIQVRIKPGSANKPKDELDYANDIEKKICKMEKCSSVFN |
| with additional C-Terminal | VVNSSIGLIMVLSFLFLNPHHHHHHAC |
| Cysteine for conjugation] | |
| R21 Peptide NANP₍₆ₓ₎ | NPNANPNANPNANPNANPNANPNA(K/Mal)-amide |
| [Peptide of N-Terminal | |
| residues of R21 6x(NANP) | |
| with NHS for conjugation] | |
| R21 Peptide NANP-2 | ANPNKNNQGNGQGHNMPNDPNRNVDENANANSAVKNNNNE(K/Mal)- |
| [Peptide of second segment of | amide |
| R21 with NHS for conjugation] | |
| R21 Peptide Solved Structure | EPSDKHIKEYLNKIQNSLSTEWSPSSVTSGNGIQVRIKPGSANKPKDE |
| [Structured portion of C- | LDYANDIEKKISKMEKSS(K/Mal)-amide |
| Terminal segment of R21] | |
| Ring 10 (Ly1) Wt protein | MPWWYRRRSYNPWRRRNWFRRPRKTIYRRYRRRRRWVRRKPFYK |
| sequence | RKIKRLNIVEWQPKSIRKCRIKGMLCLFQTTEDRLSYNFDMYEE |
| | SIIPEKLPGGGGFSIKNISLYALYQEHIHAHNIFTHINTDRPLA |
| | RYTGCSLKFYQSKDIDYVVTYSTSLPLRSSMGMYNSMQPSIHLM |
| | QQNKLIVPSKQTQKRRKPYIKKHISPPTQMKSQWYFQHNIANIP |
| | LLMIRTTALTLDNYYIGSRQLSTNVTIHTLNTTYIQNRDWGDRN |
| | KTYYCQTLGTQRYFLYGTHSTAQNINDIKLQELIPLINTQDYVQ |
| | GFDWTEKDKHNITTYKEFLTKGAGNPFHAEWITAQNPVIHTANS |
| | PTQIEQIYTASTTTFQNKKLTDLPTPGYIFITPTVSLRYNPYKD |
| | LAERNKCYFVRSKINAHGWDPEQHQELINSDLPQWLLLFGYPDY |
| | IKRTQNFALVDTNYILVDHCPYTNPEKTPFIPLSTSFIEGRSPY |
| | SPSDTHEPDEEDQNRWYPCYQYQQESINSICLSGPGTPKIPKGI |
| | TAEAKVKYSFNFKWGGDLPPMSTITNPTDQPTYVVPNNFNETTS |
| | LQNPTTRPEHFLYSFDERRGQLTEKATKRLLKDWETKETSLLST |
| | EYRFAEPTQTQAPQEDPSSEEEEESNLFERLLRQRTKQLQLKRR |
| | IIQTLKDLQKLE |
| CCN5 CTermCys | MRGTPKTHLLAFSLLCLLSKVRTQLCPTPCTCPWPPPRCPLGVPLVLD |
| | GCGCCRVCARRLGEPCDQLHVCDASQGLVCQPGAGPGGRGALCLLAED |
| | DSSCEVNGRLYREGETFQPHCSIRCRCEDGGFTCVPLCSEDVRLPSWD |
| | CPHPRRVEVLGKCCPEWVCGQGGGLGTQPLPAQGPQFSGLVSSLPPGV |
| | PCPEWSTAWGPCSTTCGLGMATRVSNQNRFCRLETQRRLCLSRPCPPS |
| | RGRSPQNSAFGGGGSGGGGSGGGGSC* |
| Aflibercept malE_CTermCys | MKIKTGARILALSALTTMMFSASALASDTGRPFVEMYSEIPEIIHMTE |
| | GRELVIPCRVTSPNITVILKKFPLDTLIPDGKRIIWDSRKGFIISNAT |
| | YKEIGLLTCEATVNGHLYKTNYLTHRQTNTIIDVVLSPSHGIELSVGE |
| | KLVLNCTARTELNVGIDFNWEYPSSKHQHKKLVNRDLKTQSGSEMKKF |
| | LSTLTIDGVTRSDQGLYTCAASSGLMTKKNSTFVRVHEKDKTHTCPPC |
| | PAPELLGGPSVFLFPPKPKDTLMISRTPEVTCVVVDVSHEDPEVKFNW |
| | YVDGVEVHNAKTKPREEQYNSTYRVVSVLTVLHQDWLNGKEYKCKVSN |
| | KALPAPIEKTISKAKGQPREPQVYTLPPSRDELTKNQVSLTCLVKGFY |
| | PSDIAVEWESNGQPENNYKTTPPVLDSDGSFFLYSKLTVDKSRWQQGN |
| | VFSCSVMHEALHNHYTQKSLSLSPGGGGSGGGGGGGGSGGGGSC |
| Aflib_D1-D2 malE_CTermCys | MKIKTGARILALSALTTMMFSASALASDTGRPFVEMYSEIPEIIHMTE |
| | GRELVIPCRVTSPNITVTLKKFPLDTLIPDGKRIIWDSRKGFIISNAT |
| | YKEIGLLTCEATVNGHLYKTNYLTHRQTNTIIDVVLSPSHGIELSVGE |
| | KLVLNCTARTELNVGIDFNWEYPSSKHQHKKLVNRDLKTQSGSEMKKF |
| | LSTLTIDGVTRSDQGLYTCAASSGLMTKKNSTFVRVHEKGGGGSGGGG |
| | SGGGGSC |
| Ranibizumab HC | MKIKTGARILALSALTTMMESASALAEVQLVESGGGLVQPGGSLRLSC |
| malE_CTermCys | AASGYDFTHYGMNWVRQAPGKGLEWVGWINTYTGEPTYAADFKRRFTF |
| | SLDTSKSTAYLQMNSLRAEDTAVYYCAKYPYYYGTSHWYFDVWGQGTL |
| | VTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSN |
| | TKVDKKVEPKSCDKTHLGGGGSGGGGSGGGGSC |
| Bevacizumab HC | MKIKTGARILALSALTTMMESASALAEVQLVESGGGLVQPGGSLRLSC |
| malE_CTermCys | AASGYTFTNYGMNWVRQAPGKGLEWVGWINTYTGEPTYAADFKRRFTF |
| | SLDTSKSTAYLQMNSLRAEDTAVYYCAKYPHYYGSSHWYFDVWGQGTL |
| | VTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSN |
| | TKVDKKVEPKSCDKTHTCPPCPAPELLGGPSVFLFPPKPKDTLMISRT |
| | PEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLP |
| | PSREEMTKNQVSLTCLVKGFYPSDIAVEWESNGQPENNYKTTPPVLDS |
| | DGSFFLYSKLTVDKSRWQQGNVFSCSVMHEALHNHYTQKSLSLSPGKK |
| | DELGGGGSGGGGSGGGGSC |
| scFab RaniHC-link50-LC | MKIKTGARILALSALTTMMESASALAEVQLVESGGGLVQPGGSLRLSC |
| malE_CTermCys | AASGYDFTHYGMNWVRQAPGKGLEWVGWINTYTGEPTYAADFKRRFTF |
| | SLDTSKSTAYLQMNSLRAEDTAVYYCAKYPYYYGTSHWYFDVWGQGTL |
| | VTVSSASTKGPSVFPLAPSSKSTSGGTAALGCLVKDYFPEPVTVSWNS |
| | GALTSGVHTFPAVLQSSGLYSLSSVVTVPSSSLGTQTYICNVNHKPSN |
| | TKVDKKVEPKSCDKTHLGGSSGSGSGSTGTSSSGTGTSAGTTGTSAST |
| | SGSGSGGGGGSGGGGSAGGDIQLTQSPSSLSASVGDRVTITCSASQDI |
| | SNYLNWYQQKPGKAPKVLIYFTSSLHSGVPSRFSGSGSGTDFTLTISS |
| | LQPEDFATYYCQQYSTVPWTFGQGTKVEIKRTVAAPSVFIFPPSDEQL |
| | KSGTASVVCLLNNFYPREAKVQWKVDNALQSGNSQESVTEQDSKDSTY |
| | SLSSTLTLSKADYEKHKVYACEVTHQGLSSPVTKSFNRGECGGGGSGG |
| | GGSGGGGSC |

### Example 6. Generation of Circular ssDNA Transgene for In Vitro Assembly of Anellovectors

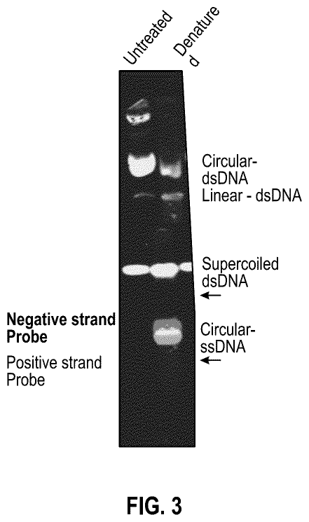

Anelloviruses encapsidate circularized negative sense (ns) single stranded (ss) DNA genomes. Described in this example is a method for generating circularized ns-ssDNA purified in vitro. First, the genome of an Anellovirus (e.g., the genome of strain Ring 2 as described herein) was generated in a plasmid for amplification. Next, the DNA encoding the viral genome was cut from the plasmid and re-ligated to form double-stranded in vitro circularized (IVC) DNA. The IVC DNA was denatured with heat by boiling at 80° C. for 5 minutes in the presence of urea and run on a denaturing gel overnight at 4° C. When the gel is stained with fluorescently-labeled primers from either the positive strand (to bind the negative-sense sequence) or the negative strand (to bind the positive-sense sequence) it was observed that the IVC DNA was separated into positive and negative sense circularized ssDNA (FIG. 3).

The ssDNA can be extracted from the gel using conventional DNA purification kits and used in anellovector in vitro encapsidation screening. Plasmids can also be cloned that encode viral genomes that also harbor reporter genes or therapeutic genes, for example, such that generation and encapsidation of the resulting circularized ssDNA would generate an anellovector that can transduce cells with the reporter or therapeutic gene.

### Example 7. Anellovector ORF1 VLP Expression in Insect or Mammalian Cells and Purification

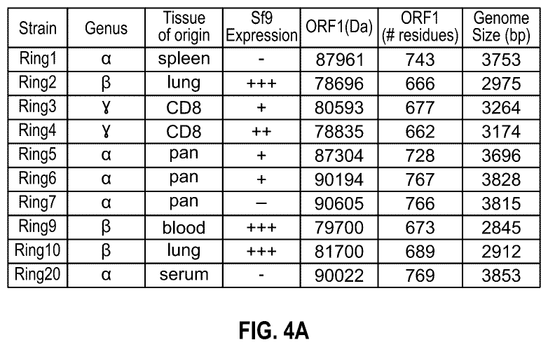

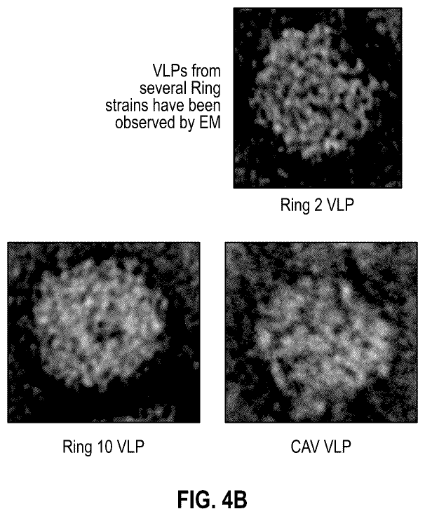

Described in this example are expression and purification protocols for Anellovirus ORF1 recombinant proteins and variations that can be used, for example, in generating anellovector strains in vitro. Several Anellovirus ORF1 strains have been successfully expressed in insect (Sf9) and/or mammalian (293) cell lines (FIG. 4A). As described in Examples 8 and 9 below, several ORF1 proteins from different Anellovirus strains (as well as the related CAV Vp1 capsid protein) were shown to have a propensity to self-assemble into VLPs in vitro when observed by electron microscopy (FIG. 4B).

In one example, anellovectors can be produced in vitro by generating expression constructs for certain Anellovirus proteins (e.g., as described herein), such as an Anellovirus ORF1. Such expression constructs can be introduced into cells (e.g., mammalian cells or insect cells, e.g. Sf9 cells) to produce Anellovirus ORF1 proteins capable of forming capsids. Production of the ORF1 proteins can be done in the presence or absence of a suitable Anellovirus ORF2 protein. The expression constructs can also be engineered to attach (e.g., fuse) an affinity tag to the Anellovirus ORF1 protein. The ORF1 proteins can then be purified from the cells.

In some examples, a desired payload nucleic acid molecule (e.g., an engineered Anellovirus genome, a wild-type Anellovirus genome, an oligonucleotide, a single-stranded DNA, a double-stranded DNA, or an RNA, e.g., an mRNA) can be encapsulated by the ORF1 proteins in vitro, under conditions suitable for assembly of the ORF1 proteins into a proteinaceous exterior (e.g., a capsid). Such conditions may include, for example, a solution comprising one or more of: detergents, buffers, and/or reducing agents (e.g., as described herein), as well as incubation at a preselected temperature for a preselected time.

This assembly can result in formation of particles (e.g., comprising assembled ORF1 proteins encapsulating the desired payload nucleic acid molecule). Particle formation can be assessed, for example, by co-migration of ssDNA in size exclusion chromatography and/or by detecting DNase-resistant payload nucleic acid molecules (e.g., by PCR), e.g., as described herein. In addition, particles can be assessed for their capacity to transduce target cells with the payload nucleic acid molecule (e.g., by detecting a gene product encoded by the payload nucleic acid molecule, e.g., a reporter).

In addition to exploring the purification and in vitro assembly of ORF1 from different strains, Anellovirus ORF1 fragments or chimeric molecules can be used to improve the efficiency of in vitro assembly. Such fragment or chimeric molecules include, but are not limited to, Anellovirus ORF1 proteins that harbor mutations introduced into the N-terminal Arginine-rich region (also referred to as the ARG arm) to alter the pI of the ARG arm, permitting pH sensitive nucleic acid binding to trigger particle assembly (SEQ ID NOs: 563-565). Anellovirus ORF1 mutations that improve stability include, for example, mutations to the interprotomer contacting beta strands F and G of the canonical jellyroll beta-barrel (F and G beta strands) to alter the hydrophobic state of the protomer surface to make capsid formation more thermodynamically favored.

Exemplary chimeric ORF1 proteins include, but are not limited to, Anellovirus ORF1 proteins which have a portion or portions of their sequence replaced with comparable portions from another capsid protein, such as BFDV, CAV capsid protein or Hepatitis E (such as the ARG arm or F and G beta strands of Ring 9 ORF1 replaced with the comparable components from BFDV capsid protein; SEQ ID NOs: 566-567). Chimeric ORF1 proteins may also include ORF1 proteins which have a portion or portions of their sequence replaced with comparable portions of another Anellovirus ORF1 protein (such as jellyroll fragments or the C-terminal portion of Ring 2 ORF1 replaced with comparable portions of Ring 9 ORF1; SEQ ID NOs: 568-575).

Proteins will be purified using purification techniques, including but not limited to chelating purification, heparin purification, gradient sedimentation purification and/or SEC purification.

### Example 8. Expression of Ring2 and Ring10 Anellovirus ORF1s in Insect Cells

In this example, DNA sequences encoding Ring 2 ORF1 or Ring 10 ORF1, each fused to an N-terminal HIS₆-tag (HIS-ORF1), was codon optimized for insect expression and cloned into the baculovirus expression vector pFASTbac system according to the manufacture's method (ThermoFisher Scientific). Insect cells (Sf9 cells) were infected with Ring HIS-ORF1 baculovirus and the cells were harvested 3-days post-infection by centrifugation. The cells were lysed and the protein was purified using a chelating resin affinity column (HisTrap, GE Healthcare). The resulting material was purified again using a heparin affinity column (Heparin HiTrap, GE Healthcare) and fractions containing ORF1 were analyzed by negative staining electron microscopy. Both Ring 2 ORF1 and Ring 10 ORF1 exhibited an observed propensity to form ˜35 nm virus-like particles (VLPs) in this in vitro setting (FIG. 4B).

### Example 9. Expression of CAV Capsid Protein VP1 in Mammalian Cells

In this example, DNA sequences encoding CAV capsid protein (CAV Vp1), fused to an N-terminal HIS₆—Flag-tag (HIS-Flag-Vp1), and helper protein (Vp2) were codon optimized for mammalian expression and cloned into a mammalian expression vector including a CMV promoter. Mammalian cells (293expi cells) were transfected with CAV Vp1 and Vp2 expression vectors. The cells were harvested 3-days post-infection by centrifugation. The cells were lysed and the lysis was purified using chelation and heparin purification as described in Example 8. The elution fraction containing CAV Vp1 were analyzed by negative staining electron microscopy. As shown in FIG. 4B, CAV Vp1 virus-like particles were observed in this in vitro setting.

### Example 10. Expression of Ring2 ORF1 and ORF2 in Insect Cells

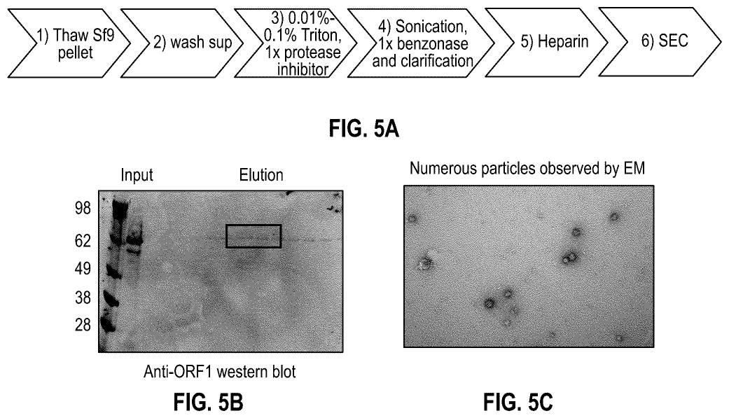

In this example, untagged Ring 2 ORF1 and ORF2 (a putative zinc-finger Ring 2 protein of unknown function) were cloned into a dual-expression baculovirus and co-expressed in Sf9 insect cells using a baculovirus system as described herein. Frozen pellet from a 1 L preparation of the Sf9 cells were resuspended in 60 mL cytosolic buffer (50 mM Tris pH 8, 50 mM NaCl, lx protease inhibiter), vortexed, pipetted up-down and distributed into two 30 mL aliquotes in 50 mL conical tubes. The solutions were spun down for 20 min at 14,000 rpm using a fixed rotor centrifuge. The supernatant was collected into separate 50 mL conical tubes (“wash sup”). Each pellet was resuspended with 30 mL lysis buffer (50 mM Tris pH 8, 50 mM NaCl buffer, lx protease inhibitor, 0.01-0.1% Triton), vortexed, and sonicated with four cycles. lx benzonase was added and the solutions were spun for 20 min at 14,000 rpm using a fixed rotor centrifuge. The lysate was incubated for about 30 minutes to allow the benzonase to react to remove host cell DNA. The ORF1 was purified from the lysates using an affinity purification step (heparin column pH=8 with a high salt gradient elution) and VLPs, partially formed VLPs, and ORF1 proteins (putative capsomers) were separated using size-exclusion purification (SEC; GE Healthcare Sephacryl 5-500 column). The resulting material was analyzed by electron microscopy (FIG. 5A-5C). Approximately 10¹¹ total VLP particles at approximately 109-10¹⁰ particles/ml concentration were purified, as determined by western blot analysis and electron microscopy. Such a high-titer VLP preparation can be used to define in vitro disassembly and re-assembly/encapsidation conditions for anellovector generation as described below.

### Example 11. Disassembly of Ring2 VLPs Using Urea

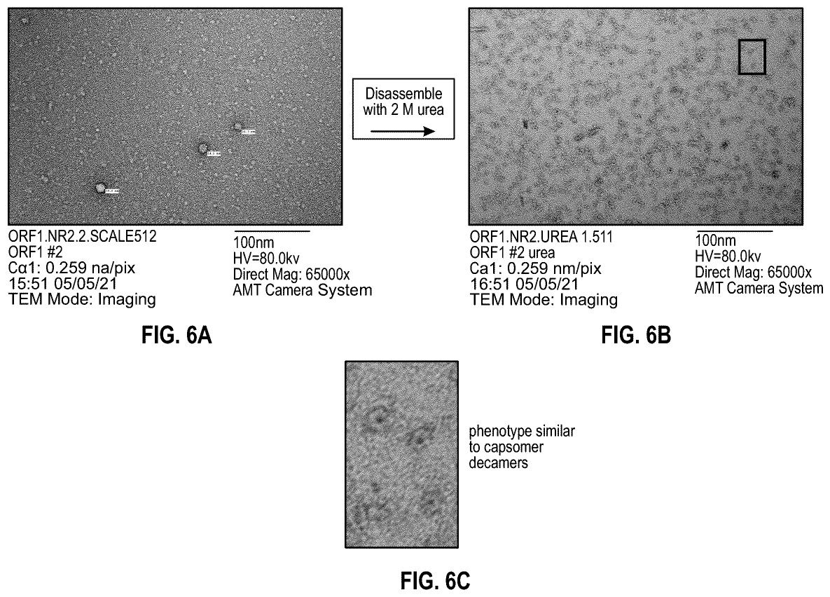

In this example, purified Ring2 VLPs were dissociated using a chaotropic agent. Briefly, purified Ring 2 VLPs were treated with urea at different concentrations, between 1 to 6 molar (M), to identify conditions sufficient to disassemble the particles. In one example, Ring 2 VLPs were treated with urea with a final concentration of 2M for approximately 10 minutes. The sample was observed by electron microscopy before (FIG. 6A) and after (FIG. 6B) urea treatment. Prior to treatment with urea, VLPs were observed with an estimated particle titer of 1×10⁹ to 1×10¹⁰ particles/ml. After treatment with urea, the VLPs were no longer observed. A new species, which appear to be hollow circles, like capsomers, was also observed (FIG. 6C).

In a further example, Ring 2 VLPs are dissociated with 2M urea, and the urea is dialyzed away in the absence or presence of mRNA encoding the mCherry reporter gene. In this example, mRNA is introduced at a molar concentration estimated to be approximately 5-times greater than the estimated number of VLPs prior to dissociation. The dissociated ORF1 and mRNA solution are incubated for approximately 30 minutes to allow complex formation before reassembly by dialysis. After the incubation period, disassembled ORF1 (i.e. VLPs treated with 2M urea) with or without mRNA are then dialyzed against 50 mM Tris pH 8.0 with 150 mM NaCl and 0.01% poloxamer to permit VLP reassembly. The initial VLPs, dissociated ORF1, reassembled ORF1 in the absence of nucleic acids, and reassembled ORF1 in the presence of nucleic acids are then screened by EM to confirm that the dissociation/re-assembly processes are successful as well as to estimate the amount of VLP recovered. It is contemplated that no VLPs will be observed for disassembled VLP or disassembled VLP dialyzed in the absence of mRNA. However, it is contemplated that VLPs prior to disassembly may be present at a particle titer of 1×109 to 1×10¹⁰particles/ml and the VLP formation observed after dialysis in the presence of mRNA may have a particle titer of 1×10⁷ to 1×10⁸particles/ml. It is contemplated that VLP reassembly occurs when disassembled ORF1 is dialyzed/reassembled in the presence of mRNA, resulting in mRNA-encapsidated anellovectors.

### Example 12. Dissociation of Anellovirus VLPs and Reassembly Around Nucleic Acid Cargoes

In this example, Anellovirus ORF1 proteins produced and purified as described herein (e.g., wildtype ORF1 protein, chimeric ORF1 protein, or fragments thereof) will be disassembled and then reassembled in vitro. VLPs will be incubated under conditions sufficient to dissociate VLPs or viral capsids (e.g., as described herein), and then under conditions suitable enable reassembly, for example, around nucleic acid cargo (FIGS. 5A-5C). Exemplary nucleic acid cargo can include, without limitation, as double stranded DNA, single stranded DNA (ssDNA), or RNA that encodes a gene of interest to be delivered as a therapeutic agent. Exemplary conditions sufficient to dissociate VLPs or viral capsids include, but are not limited to, buffers of different pH, conditions of defined conductivity (salt content), conditions containing detergents (such as SDS, Tween, Triton), conditions containing chaotropic agents (such as Urea, e.g., as described herein) or conditions involving defined temperature and time (reannealing temperatures). Generally, nucleic acid cargo of defined concentration will be combined with Anellovirus ORF1 proteins of defined concentration and treated with conditions sufficient to permit nucleic acid encapsidation. The resulting particles can be subsequently purified using viral purification procedures in the art and/or as described herein.

Encapsidation of ssDNA Cargo

In this example, purified Anellovirus ORF1 proteins are treated with high salt in 2 M Urea to disassemble VLPs into dispersed protein or capsomers. The Anellovirus ORF1 proteins are then mixed with ssDNA and dialyzed against Tris pH 8.0 with 150 mM NaCl to permit VLP formation and ssDNA encapsidation. The subsequent complex is purified by SEC using to isolate anellovectors encapsidating ssDNA from non-encapsidated DNA. Anellovector assembly can be further evaluated by biophysical assessment such as DLS or electron microscopy, e.g., as described herein.

Encapsidation of mRNA Cargo with Wild-Type Anellovirus ORF1

In this example, purified Anellovirus ORF1 proteins are treated with 1 M NaCl with 0.1% SDS dissociate oligomers or VLPs into dispersed protein or capsomers. The Anellovirus ORF1 proteins are then mixed with mRNA, such as an mRNA that encodes a gene of interest (e.g., GFP, mCherry, or EPO), and dialyzed against Tris pH 8.0 with 150 mM NaCl to permit VLP formation. The subsequent complex is purified by SEC using Tris pH 8.0 buffer to isolate anellovectors encapsidating mRNA. Anellovector assembly can be further evaluated by in vitro or in vivo readout, for example, by transducing cells and observing the expression of a reporter gene (in the case of mCherry or GFP) or through expression of a gene of interest (such as using an ELISA to detect the expression of a gene such as EPO).

Encapsidation of mRNA Cargo with Modified ORF1 Having mRNA Binding Region

In this example, packaging of mRNA cargo by Anellovirus ORF1 proteins is improved by modifying the Anellovirus ORF1 protein to harbor contact residues known to bind mRNA. For example, the ssDNA contact residues and/or the jellyroll beta strands known to contact ssDNA and/or the N-terminal arginine ARM can be replaced with components of an mRNA binding viral protein (e.g., MS2 coat protein) or other mRNA-binding protein to permit efficient binding and packaging of mRNA. This mRNA-binding chimeric ORF1 could be then treated with 1 M NaCl with 0.1% SDS dissociate oligomers or VLPs into dispersed protein or capsomers. The chimeric ORF1 would then be mixed with mRNA, such as an mRNA that translate a gene of interest such as GFP, mCherry or EPO, and dialyzed against Tris pH 8.0 with 150 mM NaCl to permit VLP formation. The subsequent complex is purified by SEC using Tris pH 8.0 buffer to isolate anellovectors encapsidating mRNA. Anellovector assembly can be further evaluated by in vitro or in vivo readout, for example, by transducing cells and observing the expression of the reporter gene (in the case of mCherry or GFP) or through expression of a gene of interest (such as using an ELISA to detect the expression of a gene such as EPO).

Encapsidation of ssDNA-mRNA Hybrid Cargo with ORF1

In this example, packaging of mRNA cargo by Anellovirus ORF1 proteins is improved by binding the mRNA molecule to ssDNA or modifying the mRNA transgene in such a way that that a section of the backbone would permit binding to the ssDNA contact residues of wildtype Anellovirus ORF1. For example, modified ssDNA that can bind Anellovirus ORF1 by virtue of its sugar-chain backbone but pair with mRNA non-covalently is mixed with the mRNA to produce a synthetic mRNA complex. Alternatively, a synthetic mRNA transgene can be synthesized with a section or sections of the mRNA molecule harboring a DNA backbone permitting binding and encapsidation with Anellovirus ORF1, while retaining the portion of the mRNA that encodes the gene to be delivered. Anellovirus ORF1 could be then treated with 1 M NaCl with 0.1% SDS dissociate oligomers or VLPs into dispersed protein or capsomers. The Anellovirus ORF1 would then be mixed with the synthetic mRNA (complexes or molecules), such as an mRNA that translate a gene of interest such as GFP, mCherry or EPO, and dialyzed against Tris pH 8.0 with 150 m NaCi to permit VLP formation. The subsequent particle is purified by SEC using Tris pH 8.0 buffer to isolate anellovectors encapsidating mRNA. Anellovector assembly can be further evaluated by in vitro or in vivo readout by transducing cells and observing the expression of the reporter gene (in the case of mCherry or GFP) or through expression of a gene of interest (such as using an ELISA to detect the expression of a gene such as EPO).

Example 13. Exemplary sequences for ORF1 molecules

| Sequence Name | Sequence | SEQ ID NO: |
|---|---|---|
| Ring 2 N- | MGSSHHHHHHGSDYKDDDDKSGSLEVLFQGPSGMPYYYRRR | 561 |
| terminal HIS- | RYNYRRPRWYGRGWIRRPFRRRFRRKRRVRPTYTTIPLKQW | |
| FLAG- | QPPYKRTCYIKGQDCLIYYSNLRLGMNSTMYEKSIVPVHWP | |
| 3CProtease- | GGGSFSVSMLTLDALYDIHKLCRNWWTSTNQDLPLVRYKGC | |
| ORF1 | KITFYQSTFTDYIVRIHTELPANSNKLTYPNTHPLMMMMSK | |
| | YKHIIPSRQTRRKKKPYTKIFVKPPPQFENKWYFATDLYKI | |
| | PLLQIHCTACNLQNPFVKPDKLSNNVTLWSLNTISIQNRNM | |
| | SVDQGQSWPFKILGTQSFYFYFYTGANLPGDTTQIPVADLL | |
| | PLINPRINRPGQSLNEAKITDHITFTEYKNKFTNYWGNPFN | |
| | KHIQEHLDMILYSLKSPEAIKNEWTTENMKWNQLNNAGTMA | |
| | LTPFNEPIFTQIQYNPDRDTGEDTQLYLLSNATGTGWDPPG | |
| | IPELILEGFPLWLIYWGFADFQKNLKKVINIDTNYMLVAKT | |
| | KFTQKPGTFYLVILNDTFVEGNSPYEKQPLPEDNIKWYPQV | |
| | QYQLEAQNKLLQTGPFTPNIQGQLSDNISMFYKFYFKWGGS | |
| | PPKAINVENPAHQIQYPIPRNEHETTSLQSPGEAPESILYS | |
| | FDYRHGNYTTTALSRISQDWALKDTVSKITEPDRQQLLKQA | |
| | LECLQISEETQEKKEKEVQQLISNLRQQQQLYRERIISLLK | |
| | DQ | |
| Ring 9 N- | MGSSHHHHHHGSDYKDDDDKSGSLEVLFQGPSGMPPYWRQK | 562 |
| terminal HIS- | YYRRRYRPFSWRTRRIIQRRKRWRYRKPRKTYWRRKLRVRK | |
| FLAG- | RFYKRKLKKIVLKQFQPKIIRRCTIFGTICLFQGSPERANN | |
| 3CProtease- | NYIQTIYSYVPDKEPGGGGWTLITESLSSLWEDWEHLKNVW | |
| ORF1 | TQSNAGLPLVRYGGVTLYFYQSAYTDYIAQVFNCYPMTDTK | |
| | YTHADSAPNRMLLKKHVIRVPSRETRKKRKPYKRVRVGPPS | |
| | QMQNKWYFQRDICEIPLIMIAATAVDFRYPFCASDCASNNL | |
| | TLTCLNPLLFQNQDFDHPSDTQGYFPKPGVYLYSTQRSNKP | |
| | SSSDCIYLGNTKDNQEGKSASSLMTLKTQKITDWGNPFWHY | |
| | YIDGSKKIFSYFKPPSQLDSSDFEHMTELAEPMFIQVRYNP | |
| | ERDTGQGNLIYVTENFRGQHWDPPSSDNLKLDGFPLYDMCW | |
| | GFIDWIEKVHETENLLTNYCFCIRSSAFNEKKTVFIPVDHS | |
| | FLTGFSPYETPVKSSDQAHWHPQIRFQTKSINDICLTGPGC | |
| | ARSPYGNYMQAKMSYKFHVKWGGCPKTYEKPYDPCSQPNWT | |
| | IPHNLNETIQIQNPNTCPQTELQEWDWRRDIVTKKAIERIR | |
| | QHTEPHETLQISTGSKHNPPVHRQTSPWTDSETDSEEEKDQ | |
| | TQEIQIQLNKLRKHQQHLKQQLKQYLKPQNIE | |
| Ring 2 ORF1 | MGSSHHHHHHGSDYKDDDDKSGSLEVLFQGPSGMPPYWRQK | 563 |
| with ARG arm | YYRRRYRPFSWRTRRIIQRRKRWRYRKPRKTYWRRKLRVRK | |
| of Ring 9 (Ring | RRVRPTYTTIPLKQWQPPYKRTCYIKGQDCLIYYSNLRLGM | |
| 291) | NSTMYEKSIVPVHWPGGGSFSVSMLTLDALYDIHKLCRNWW | |
| | TSTNQDLPLVRYKGCKITFYQSTFTDYIVRIHTELPANSNK | |
| | LTYPNTHPLMMMMSKYKHIIPSRQTRRKKKPYTKIFVKPPP | |
| | QFENKWYFATDLYKIPLLQIHCTACNLQNPFVKPDKLSNNV | |
| | TLWSLNTISIQNRNMSVDQGQSWPFKILGTQSFYFYFYTGA | |
| | NLPGDTTQIPVADLLPLINPRINRPGQSLNEAKITDHITFT | |
| | EYKNKFTNYWGNPFNKHIQEHLDMILYSLKSPEAIKNEWTT | |
| | ENMKWNQLNNAGTMALTPFNEPIFTQIQYNPDRDTGEDTQL | |
| | YLLSNATGTGWDPPGIPELILEGFPLWLIYWGFADFQKNLK | |
| | KVTNIDTNYMLVAKTKFTQKPGTFYLVILNDTFVEGNSPYE | |
| | KQPLPEDNIKWYPQVQYQLEAQNKLLQTGPFTPNIQGQLSD | |
| | NISMFYKFYFKWGGSPPKAINVENPAHQIQYPIPRNEHETT | |
| | SLQSPGEAPESILYSFDYRHGNYTTTALSRISQDWALKDTV | |
| | SKITEPDRQQLLKQALECLQISEETQEKKEKEVQQLISNLR | |
| | QQQQLYRERIISLLKDQ | |
| Ring 2 ORF1 | MGSSHHHHHHGSDYKDDDDKSGSLEVLFQGPSGMPPYWRQK | 564 |
| with ARG arm | YYRRRYRPFSWRTRRIIQRRKRWRYRKPRKTYWRRKLRVRK | |
| and Beta strands | RFYKRKLKKIVLKQFQPKIIRRCTIFGTICLFQGSNLRLGM | |
| 1 + 2 722 | NSTMYEKSIVPVHWPGGGSFSVSMLTLDALYDIHKLCRNWW | |
| epitope of Ring | TSTNQDLPLVRYKGCKITFYQSTFTDYIVRIHTELPANSNK | |
| 9 (Ring 292) | LTYPNTHPLMMMMSKYKHIIPSRQTRRKKKPYTKIFVKPPP | |
| | QFENKWYFATDLYKIPLLQIHCTACNLQNPFVKPDKLSNNV | |
| | TLWSLNTISIQNRNMSVDQGQSWPFKILGTQSFYFYFYTGA | |
| | NLPGDTTQIPVADLLPLTNPRINRPGQSLNEAKITDHITFT | |
| | EYKNKFTNYWGNPFNKHIQEHLDMILYSLKSPEAIKNEWTT | |
| | ENMKWNQLNNAGTMALTPFNEPIFTQIQYNPDRDTGEDTQL | |
| | YLLSNATGTGWDPPGIPELILEGFPLWLIYWGFADFQKNLK | |
| | KVTNIDTNYMLVAKTKFTQKPGTFYLVILNDTFVEGNSPYE | |
| | KQPLPEDNIKWYPQVQYQLEAQNKLLQTGPFTPNIQGQLSD | |
| | NISMFYKFYFKWGGSPPKAINVENPAHQIQYPIPRNEHETT | |
| | SLQSPGEAPESILYSFDYRHGNYTTTALSRISQDWALKDTV | |
| | SKITEPDRQQLLKQALECLQISEETQEKKEKEVQQLISNLR | |
| | QQQQLYRERIISLLKDQ | |
| Ring 9 with | MGSSHHHHHHGSDYKDDDDKSGSLEVLFQGPSGMPPYWRQK | 565 |
| LYS/HIS | YYRRRYRPFSWRTRRIIQRRHRWRYRKPRHTYWRRHLRVRH | |
| mutations in | RFYHRHLHHIVLKQFQPKIIRRCTIFGTICLFQGSPERANN | |
| ARG arm and | NYIQTIYSYVPDKEPGGGGWTLITESLSSLWEDWEHLKNVW | |
| first beta strand | TQSNAGLPLVRYGGVTLYFYQSAYTDYIAQVFNCYPMTDTK | |
| | YTHADSAPNRMLLKKHVIRVPSRETRKKRKPYKRVRVGPPS | |
| | QMQNKWYFQRDICEIPLIMIAATAVDFRYPFCASDCASNNL | |
| | TLTCLNPLLFQNQDFDHPSDTQGYFPKPGVYLYSTQRSNKP | |
| | SSSDCIYLGNTKDNQEGKSASSLMTLKTQKITDWGNPFWHY | |
| | YIDGSKKIFSYFKPPSQLDSSDFEHMTELAEPMFIQVRYNP | |
| | ERDTGQGNLIYVTENFRGQHWDPPSSDNLKLDGFPLYDMCW | |
| | GFIDWIEKVHETENLLTNYCFCIRSSAFNEKKTVFIPVDHS | |
| | FLTGFSPYETPVKSSDQAHWHPQIRFQTKSINDICLTGPGC | |
| | ARSPYGNYMQAKMSYKFHVKWGGCPKTYEKPYDPCSQPNWT | |
| | IPHNLNETIQIQNPNTCPQTELQEWDWRRDIVTKKAIERIR | |
| | QHTEPHETLQISTGSKHNPPVHRQTSPWTDSETDSEEEKDQ | |
| | TQEIQIQLNKLRKHQQHLKQQLKQYLKPQNIE | |
| Ring 9 with | MGSSHHHHHHGSDYKDDDDKSGSLEVLFQGPSGMWGTSNCA | 566 |
| ARG arm of | CAKFQIRRRYARPYRRRHIRRYRRRRRHFRRRRFTTNRRKR | |
| BFDV | FYKRKLKKIVLKQFQPKIIRRCTIFGTICLFQGSPERANNN | |
| | YIQTIYSYVPDKEPGGGGWTLITESLSSLWEDWEHLKNVWT | |
| | QSNAGLPLVRYGGVTLYFYQSAYTDYIAQVFNCYPMTDTKY | |
| | THADSAPNRMLLKKHVIRVPSRETRKKRKPYKRVRVGPPSQ | |
| | MQNKWYFQRDICEIPLIMIAATAVDFRYPFCASDCASNNLT | |
| | LTCLNPLLFQNQDFDHPSDTQGYFPKPGVYLYSTQRSNKPS | |
| | SSDCIYLGNTKDNQEGKSASSLMTLKTQKITDWGNPFWHYY | |
| | IDGSKKIFSYFKPPSQLDSSDFEHMTELAEPMFIQVRYNPE | |
| | RDTGQGNLIYVTENFRGQHWDPPSSDNLKLDGFPLYDMCWG | |
| | FIDWIEKVHETENLLTNYCFCIRSSAFNEKKTVFIPVDHSF | |
| | LTGFSPYETPVKSSDQAHWHPQIRFQTKSINDICLTGPGCA | |
| | RSPYGNYMQAKMSYKFHVKWGGCPKTYEKPYDPCSQPNWTI | |
| | PHNLNETIQIQNPNTCPQTELQEWDWRRDIVTKKAIERIRQ | |
| | HTEPHETLQISTGSKHNPPVHRQTSPWTDSETDSEEEKDQT | |
| | QEIQIQLNKLRKHQQHLKQQLKQYLKPQNIE | |
| Ring 9 with beta | MGSSHHHHHHGSDYKDDDDKSGSLEVLFQGPSGMPPYWRQK | 567 |
| strands Fand G | YYRRRYRPFSWRTRRIIQRRKRWRYRKPRKTYWRRKLRVRK | |
| of BFDV capsid | RFYKRKLKKIVLKQFQPKIIRRCTIFGTICLFQGSPERANN | |
| protein | NYIQTIYSYVPDKEPGGGGWTLITESLSSLWEDWEHLKNVW | |
| | TQSNAGLPLVRYGGVTLYFYQSAYTDYIAQVENCYPMTDTK | |
| | YTHADSAPNRMLLKKHAKKWFSRETRKKRKPGFKRLLGPPS | |
| | QMQNKWYFQRDICEIPLIMIAATAVDFRYPFCASDCASNNL | |
| | TLTCLNPLLFQNQDFDHPSDTQGYFPKPGVYLYSTQRSNKP | |
| | SSSDCIYLGNTKDNQEGKSASSLMTLKTQKITDWGNPFWHY | |
| | YIDGSKKIFSYFKPPSQLDSSDFEHMTELAEPMFIQVRYNP | |
| | ERDTGQGNLIYVTENFRGQHWDPPSSDNLKLDGFPLYDMCW | |
| | GFIDWIEKVHETENLLTNYCFCIRSSAFNEKKTVFIPVDHS | |
| | FLTGFSPYETPVKSSDQAHWHPQIRFQTKSINDICLTGPGC | |
| | ARSPYGNYMQAKMSYKFHVKWGGCPKTYEKPYDPCSQPNWT | |
| | IPHNLNETIQIQNPNTCPQTELQEWDWRRDIVTKKAIERIR | |
| | QHTEPHETLQISTGSKHNPPVHRQTSPWTDSETDSEEEKDQ | |
| | TQEIQIQLNKLRKHQQHLKQQLKQYLKPQNIE | |
| Ring 2 with beta | MGSSHHHHHHGSDYKDDDDKSGSLEVLFQGPSGMPYYYRRR | 568 |
| C of Ring 9 | RYNYRRPRWYGRGWIRRPFRRRFRRKRRVRPTYTTIPLKQW | |
| | QPPYKRRCTIFGTICLFQGSNLRLGMNSTMYEKSIVPVHWP | |
| | GGGSFSVSMLTLDALYDIHKLCRNWWTSTNQDLPLVRYKGC | |
| | KITFYQSTFTDYIVRIHTELPANSNKLTYPNTHPLMMMMSK | |
| | YKHIIPSRQTRRKKKPYTKIFVKPPPQFENKWYFATDLYKI | |
| | PLLQIHCTACNLQNPFVKPDKLSNNVTLWSLNTISIQNRNM | |
| | SVDQGQSWPFKILGTQSFYFYFYTGANLPGDTTQIPVADLL | |
| | PLINPRINRPGQSLNEAKITDHITFTEYKNKFTNYWGNPEN | |
| | KHIQEHLDMILYSLKSPEAIKNEWTTENMKWNQLNNAGTMA | |
| | LTPFNEPIFTQIQYNPDRDTGEDTQLYLLSNATGTGWDPPG | |
| | IPELILEGFPLWLIYWGFADFQKNLKKVTNIDTNYMLVAKT | |
| | KFTQKPGTFYLVILNDTFVEGNSPYEKQPLPEDNIKWYPQV | |
| | QYQLEAQNKLLQTGPFTPNIQGQLSDNISMFYKFYFKWGGS | |
| | PPKAINVENPAHQIQYPIPRNEHETTSLQSPGEAPESILYS | |
| | FDYRHGNYTTTALSRISQDWALKDTVSKITEPDRQQLLKQA | |
| | LECLQISEETQEKKEKEVQQLISNLRQQQQLYRERIISLLK | |
| | DQ | |
| Ring 2 with | MGSSHHHHHHGSDYKDDDDKSGSLEVLFQGPSGMPYYYRRR | 569 |
| linker 1 of Ring | RYNYRRPRWYGRGWIRRPFRRRFRRKRRVRPTYTTIPLKQW | |
| 9 | QPPYKRTCYIKGQDCLIYYSPERANNNYIQTIYSYVPDKEP | |
| | GGGGWTLITESLSSLWEDWEHLKNVWTQSNAGLPLVRYKGC | |
| | KITFYQSTFTDYIVRIHTELPANSNKLTYPNTHPLMMMMSK | |
| | YKHIIPSRQTRRKKKPYTKIFVKPPPQFENKWYFATDLYKI | |
| | PLLQIHCTACNLQNPFVKPDKLSNNVTLWSLNTISIQNRNM | |
| | SVDQGQSWPFKILGTQSFYFYFYTGANLPGDTTQIPVADLL | |
| | PLINPRINRPGQSLNEAKITDHITFTEYKNKFTNYWGNPFN | |
| | KHIQEHLDMILYSLKSPEAIKNEWTTENMKWNQLNNAGTMA | |
| | LTPFNEPIFTQIQYNPDRDIGEDTQLYLLSNATGTGWDPPG | |
| | IPELILEGFPLWLIYWGFADFQKNLKKVINIDTNYMLVAKT | |
| | KFTQKPGTFYLVILNDTFVEGNSPYEKQPLPEDNIKWYPQV | |
| | QYQLEAQNKLLQTGPFTPNIQGQLSDNISMFYKFYFKWGGS | |
| | PPKAINVENPAHQIQYPIPRNEHETTSLQSPGEAPESILYS | |
| | FDYRHGNYTTTALSRISQDWALKDTVSKITEPDRQQLLKQA | |
| | LECLQISEETQEKKEKEVQQLISNLRQQQQLYRERIISLLK | |
| | DQ | |
| Ring 2 with beta | MGSSHHHHHHGSDYKDDDDKSGSLEVLFQGPSGMPYYYRRR | 570 |
| strand D of | RYNYRRPRWYGRGWIRRPFRRRFRRKRRVRPTYTTIPLKQW | |
| Ring 9 | QPPYKRTCYIKGQDCLIYYSNLRLGMNSTMYEKSIVPVHWP | |
| | GGGSFSVSMLTLDALYDIHKLCRNWWTSTNQDLPLVRYGGV | |
| | TLYFYQSTFTDYIVRIHTELPANSNKLTYPNTHPLMMMMSK | |
| | YKHIIPSRQTRRKKKPYTKIFVKPPPQFENKWYFATDLYKI | |
| | PLLQIHCTACNLQNPFVKPDKLSNNVTLWSLNTISIQNRNM | |
| | SVDQGQSWPFKILGTQSFYFYFYTGANLPGDTTQIPVADLL | |
| | PLINPRINRPGQSLNEAKITDHITFTEYKNKFTNYWGNPFN | |
| | KHIQEHLDMILYSLKSPEAIKNEWTTENMKWNQLNNAGTMA | |
| | LTPFNEPIFTQIQYNPDRDTGEDTQLYLLSNATGTGWDPPG | |
| | IPELILEGFPLWLIYWGFADFQKNLKKVTNIDTNYMLVAKT | |
| | KFTQKPGTFYLVILNDTFVEGNSPYEKQPLPEDNIKWYPQV | |
| | QYQLEAQNKLLQTGPFTPNIQGQLSDNISMFYKFYFKWGGS | |
| | PPKAINVENPAHQIQYPIPRNEHETTSLQSPGEAPESILYS | |
| | FDYRHGNYTTTALSRISQDWALKDTVSKITEPDRQQLLKQA | |
| | LECLQISEETQEKKEKEVQQLISNLRQQQQLYRERIISLLK | |
| | DQ | |
| Ring 2 with | MGSSHHHHHHGSDYKDDDDKSGSLEVLFQGPSGMPYYYRRR | 571 |
| linker 2 of Ring | RYNYRRPRWYGRGWIRRPFRRRFRRKRRVRPTYTTIPLKQW | |
| 9 | QPPYKRTCYIKGQDCLIYYSNLRLGMNSTMYEKSIVPVHWP | |
| | GGGSFSVSMLTLDALYDIHKLCRNWWTSTNQDLPLVRYKGC | |
| | KITFYQSTFTDYIVRIHNCYPMTDTKYTHADSAPNRMLLKK | |
| | HKHIIPSRQTRRKKKPYTKIFVKPPPQFENKWYFATDLYKI | |
| | PLLQIHCTACNLQNPFVKPDKLSNNVTLWSLNTISIQNRNM | |
| | SVDQGQSWPFKILGTQSFYFYFYTGANLPGDTTQIPVADLL | |
| | PLINPRINRPGQSLNEAKITDHITFTEYKNKFTNYWGNPFN | |
| | KHIQEHLDMILYSLKSPEAIKNEWTTENMKWNQLNNAGTMA | |
| | LTPFNEPIFTQIQYNPDRDTGEDTQLYLLSNATGTGWDPPG | |
| | IPELILEGFPLWLIYWGFADFQKNLKKVINIDTNYMLVAKT | |
| | KFTQKPGTFYLVILNDTFVEGNSPYEKQPLPEDNIKWYPQV | |
| | QYQLEAQNKLLQTGPFTPNIQGQLSDNISMFYKFYFKWGGS | |
| | PPKAINVENPAHQIQYPIPRNEHETTSLQSPGEAPESILYS | |
| | FDYRHGNYTTTALSRISQDWALKDTVSKITEPDRQQLLKQA | |
| | LECLQISEETQEKKEKEVQQLISNLRQQQQLYRERIISLLK | |
| | DQ | |
| Ring 2 with beta | MGSSHHHHHHGSDYKDDDDKSGSLEVLFQGPSGMPYYYRRR | 572 |
| strand G DNA | RYNYRRPRWYGRGWIRRPFRRRFRRKRRVRPTYTTIPLKQW | |
| binding of Ring | QPPYKRTCYIKGQDCLIYYSNLRLGMNSTMYEKSIVPVHWP | |
| 9 | GGGSFSVSMLTLDALYDIHKLCRNWWTSTNQDLPLVRYKGC | |
| | KITFYQSTFTDYIVRIHTELPANSNKLTYPNTHPLMMMMSK | |
| | YKHIIPSRQTRRKKKPYKRVRVKPPPQFENKWYFATDLYKI | |
| | PLLQIHCTACNLQNPFVKPDKLSNNVTLWSLNTISIQNRNM | |
| | SVDQGQSWPFKILGTQSFYFYFYTGANLPGDTTQIPVADLL | |
| | PLTNPRINRPGQSLNEAKITDHITFTEYKNKFTNYWGNPFN | |
| | KHIQEHLDMILYSLKSPEAIKNEWTTENMKWNQLNNAGTMA | |
| | LTPFNEPIFTQIQYNPDRDIGEDTQLYLLSNATGTGWDPPG | |
| | IPELILEGFPLWLIYWGFADFQKNLKKVINIDTNYMLVAKT | |
| | KFTQKPGTFYLVILNDTFVEGNSPYEKQPLPEDNIKWYPQV | |
| | QYQLEAQNKLLQTGPFTPNIQGQLSDNISMFYKFYFKWGGS | |
| | PPKAINVENPAHQIQYPIPRNEHETTSLQSPGEAPESILYS | |
| | FDYRHGNYTTTALSRISQDWALKDTVSKITEPDRQQLLKQA | |
| | LECLQISEETQEKKEKEVQQLISNLRQQQQLYRERIISLLK | |
| | DQ | |
| Ring 2 with beta | MGSSHHHHHHGSDYKDDDDKSGSLEVLFQGPSGMPYYYRRR | 573 |
| strand F | RYNYRRPRWYGRGWIRRPFRRRFRRKRRVRPTYTTIPLKQW | |
| interprotomer | QPPYKRTCYIKGQDCLIYYSNLRLGMNSTMYEKSIVPVHWP | |
| contact of Ring | GGGSFSVSMLTLDALYDIHKLCRNWWTSTNQDLPLVRYKGC | |
| 9 | KITFYQSTFTDYIVRIHTELPANSNKLTYPNTHPLMMMMSK | |
| | HVIRVPSRQTRRKKKPYTKIFVKPPPQFENKWYFATDLYKI | |
| | PLLQIHCTACNLQNPFVKPDKLSNNVTLWSLNTISIQNRNM | |
| | SVDQGQSWPFKILGTQSFYFYFYTGANLPGDTTQIPVADLL | |
| | PLINPRINRPGQSLNEAKITDHITFTEYKNKFTNYWGNPFN | |
| | KHIQEHLDMILYSLKSPEAIKNEWTTENMKWNQLNNAGTMA | |
| | LTPFNEPIFTQIQYNPDRDTGEDTQLYLLSNATGTGWDPPG | |
| | IPELILEGFPLWLIYWGFADFQKNLKKVINIDTNYMLVAKT | |
| | KFTQKPGTFYLVILNDTFVEGNSPYEKQPLPEDNIKWYPQV | |
| | QYQLEAQNKLLQTGPFTPNIQGQLSDNISMFYKFYFKWGGS | |
| | PPKAINVENPAHQIQYPIPRNEHETTSLQSPGEAPESILYS | |
| | FDYRHGNYTTTALSRISQDWALKDTVSKITEPDRQQLLKQA | |
| | LECLQISEETQEKKEKEVQQLISNLRQQQQLYRERIISLLK | |
| | DQ | |
| Ring 2 ORF1 | MGSSHHHHHHGSDYKDDDDKSGSLEVLFQGPSGMPYYYRRR | 574 |
| with strand H | RYNYRRPRWYGRGWIRRPFRRRFRRKRRVRPTYTTIPLKQW | |
| and I with C- | QPPYKRTCYIKGQDCLIYYSNLRLGMNSTMYEKSIVPVHWP | |
| terminal | GGGSFSVSMLTLDALYDIHKLCRNWWTSTNQDLPLVRYKGC | |
| fragment of | KITFYQSTFTDYIVRIHTELPANSNKLTYPNTHPLMMMMSK | |
| Ring 9 | YKHIIPSRQTRRKKKPYTKIFVKPPPQFENKWYFATDLYKI | |
| | PLIMIAATAVDFRYPFCASDCASNNLTLTCLNPLLFQNQDE | |
| | DHPSDTQGYFPKPGVYLYSTQRSNKPSSSDCIYLGNTKDNQ | |
| | EGKSASSLMTLKTQKITDWGNPFWHYYIDGSKKIFSYFKPP | |
| | SQLDSSDFEHMTELAEPMFIQVRYNPERDTGQGNLIYVTEN | |
| | FRGQHWDPPSSDNLKLDGFPLYDMCWGFIDWIEKVHETENL | |
| | LTNYCFCIRSSAFNEKKTVFIPVDHSFLTGFSPYETPVKSS | |
| | DQAHWHPQIRFQTKSINDICLTGPGCARSPYGNYMQAKMSY | |
| | KFHVKWGGCPKTYEKPYDPCSQPNWTIPHNLNETIQIQNPN | |
| | TCPQTELQEWDWRRDIVTKKAIERIRQHTEPHETLQISTGS | |
| | KHNPPVHRQTSPWTDSETDSEEEKDQTQEIQIQLNKLRKHQ | |
| | QHLKQQLKQYLKPQNIE | |
| Ring 2 ORF1 | MGSSHHHHHHGSDYKDDDDKSGSLEVLFQGPSGMPYYYRRR | 575 |
| with strand I | RYNYRRPRWYGRGWIRRPFRRRFRRKRRVRPTYTTIPLKQW | |
| and C-terminal | QPPYKRTCYIKGQDCLIYYSNLRLGMNSTMYEKSIVPVHWP | |
| fragment of | GGGSFSVSMLTLDALYDIHKLCRNWWTSTNQDLPLVRYKGC | |
| Ring 9 | KITFYQSTFTDYIVRIHTELPANSNKLTYPNTHPLMMMMSK | |
| | YKHIIPSRQTRRKKKPYTKIFVKPPPQFENKWYFATDLYKI | |
| | PLLQIHCTACNLQNPFVKPDKLSNNLTLTCLNPLLFQNQDE | |
| | DHPSDTQGYFPKPGVYLYSTQRSNKPSSSDCIYLGNTKDNQ | |
| | EGKSASSLMTLKTQKITDWGNPFWHYYIDGSKKIFSYFKPP | |
| | SQLDSSDFEHMTELAEPMFIQVRYNPERDIGQGNLIYVTEN | |
| | FRGQHWDPPSSDNLKLDGFPLYDMCWGFIDWIEKVHETENL | |
| | LTNYCFCIRSSAFNEKKTVFIPVDHSFLTGFSPYETPVKSS | |
| | DQAHWHPQIRFQTKSINDICLTGPGCARSPYGNYMQAKMSY | |
| | KFHVKWGGCPKTYEKPYDPCSQPNWTIPHNLNETIQIQNPN | |
| | TCPQTELQEWDWRRDIVTKKAIERIRQHTEPHETLQISTGS | |
| | KHNPPVHRQTSPWTDSETDSEEEKDQTQEIQIQLNKLRKHQ | |
| | QHLKQQLKQYLKPQNIE | |

### Example 14: Production of Anellovirus proteins in a baculovirus expression system

In this example, a baculovirus expression system from Thermofisher Scientific (Cat. no. A38841) was adapted for expression of Anellovirus proteins. Briefly, a gene of interest (e.g., a gene encoding an Anellovirus ORF as described herein) was cloned into the pFastBac plasmid, which was then transformed into DH1OBac E. coli cells harboring a baculovirus genome. The transformants were grown on indicator plates according to the manufacturer's instructions and white colonies were selected for liquid culture and extraction of bacmid DNA. Recombination of the Anellovirus ORFs into the bacmids was validated by PCR.

Validated bacmid constructs showing successful recombination of the anellovirus ORF gene were then transfected into ExpiSf9 insect cells. The cells were incubated in a 27° C. non-humidified, non-CO₂ atmosphere incubator on an orbital shaker set at 125 rpm. After 72 hours post-transfection, Passage 0 stock (P0) recombinant baculovirus was harvested from the supernatant.

ExpiSf9 cells were infected using 25-100 L of P0 baculovirus stock to make Passage 1 (P1) baculovirus for protein production. After 96 hours (approx. 4 days) post-infection, the supernatant was collected to obtain P1 baculovirus.

P1 recombinant baculovirus was titered by preparing five 10-fold serial dilutions of the test virus in fresh ExpiSf CD Medium in 1200 L total volume. 800 L of Expisf9 cells at 1.25×10⁶ viable cells/mL were seeded in a deep well plate and 1000 L of the different dilutions of the test virus were added to each well. One well was setup as a negative control. Plate was then incubated overnight at 27° C. in a non-humidified incubator on a shaking platform at 225±5 rpm. After approx. 14-16 hours of incubation, the plate was removed from the incubator and everything was transferred to microcentrifuge tubes and spun at 300×g for 5 minutes. Supernatant was aspirated and each cell pellet was resuspended in 100 L of dilution buffer (PBS+2% Fetal Bovine Serum) containing Anti-Baculovirus Envelope gp64 APC antibody at a final concentration of 0.15 g/mL. Tubes were incubated for 30 mins at room temperature. Samples were then washed with 1 mL PBS followed by a 10 min centrifuge spin at 300×g. Supernatant was aspirated and cell pellet was resuspended in 1 mL Dilution Buffer. Samples were analyzed on a flow cytometer using the following parameters: red laser excitation: 633-647 nm; emission: 660 nm. Samples with different viral dilutions expressing percent positive gp64 were noted and used to calculate the viral titer.

For this and following examples, a series of recombinant bacmids and baculovirus vectors was produced for expression using the method described above. As shown in Table E2 below, various ORFs from LY2, tth8, and other anellovirus strains were cloned into bacmids. The ORFs were either tagged with an N-terminal His-tag with or without a human rhinovirus 3C (HRV 3C) proteolytic cleavage site, a C-terminal His-tag, or were left untagged, as indicated.

TABLE E2
Recombinant bacmid constructs produced.

| Construct #/name | Strain | Ring # | ORF | Tag type | Tag position | pFastBac | Bacmid | Baculovirus | Made |
|---|---|---|---|---|---|---|---|---|---|
| tth8 ORF1 | tth8 | Ring1 | ORF1 | no-tag | NA | Yes | Yes | No | in-house |
| tth8 ORF1 N-His | tth8 | Ring1 | ORF1 | 6xHis | N-ter | Yes | Yes | Yes | in-house |
| tth8 ORF1 C-His | tth8 | Ring1 | ORF1 | 6xHis | C-ter | Yes | Yes | Yes | in-house |
| tth8 ORF2 | tth8 | Ring1 | ORF2 | no-tag | NA | Yes | Yes | Yes | in-house |
| tth8 ORF2 C-His | tth8 | Ring1 | ORF2 | 6xHis | C-ter | Yes | Yes | Yes | in-house |
| tth8 ORF1/1 | tth8 | Ring1 | ORF1/1 | no-tag | NA | Yes | Yes | Yes | in-house |
| tth8 ORF1/1 C-His | tth8 | Ring1 | ORF1/1 | 6xHis | C-ter | Yes | Yes | Yes | in-house |
| tth8 ORF1/2 | tth8 | Ring1 | ORF1/2 | no-tag | NA | Yes | Yes | Yes | in-house |
| tth8 ORF1/2 C-His | tth8 | Ring1 | ORF1/2 | 6xHis | C-ter | Yes | Yes | Yes | in-house |
| tth8 ORF2/2 | tth8 | Ring1 | ORF2/2 | no-tag | NA | Yes | Yes | Yes | in-house |
| tth8 ORF2/2 C-His | tth8 | Ring1 | ORF2/2 | 6xHis | C-ter | Yes | Yes | Yes | in-house |
| tth8 ORF2/3 | tth8 | Ring1 | ORF2/3 | no-tag | NA | Yes | Yes | Yes | in-house |
| tth8 ORF2/3 C-His | tth8 | Ring1 | ORF2/3 | 6xHis | C-ter | Yes | Yes | Yes | in-house |
| tth8 FullORF | tth8 | Ring1 | FullORF | no-tag | NA | Yes | Yes | Yes | in-house |
| tth8 FullORF C-His | tth8 | Ring1 | FullORF | 6xHis | C-ter | Yes | Yes | Yes | in-house |
| tth8 ORF2 C-His | tth8 | Ring1 | ORF2/ORF1 | 6xHis | C-ter | Yes | Yes | Yes | in-house |
| Ring 3.1 ORF1 | 6B.CD8. | Ring3.1 | ORF1 | no-tag | NA | No | No | No | in-house |
| | contig3 | | | | | | | | |
| Ring 3.1 ORF1 | 6B.CD8. | Ring3.1 | ORF1 | 6xHis | C-ter | Yes | Yes | Yes | in-house |
| C-His | contig3 | | | | | | | | |
| Ring 3.1 | 6B.CD8. | Ring3.1 | ORF2 | no-tag | NA | No | No | No | in-house |
| ORF2 | contig3 | | | | | | | | |
| Ring 3.1 | 6B.CD8. | Ring3.1 | ORF2 | 6xHis | C-ter | Yes | Yes | Yes | in-house |
| ORF2 C-His | contig3 | | | | | | | | |
| Ring 3.1 ORF2/ORF1 | 6B.CD8. | Ring3.1 | ORF2/ORF1 | 6xHis | C-ter | Yes | Yes | Yes | in-house |
| C-His | contig3 | | | | | | | | |
| LY2 FullORF | LY2 | Ring2 | FullORF | no-tag | NA | Yes | Yes | No | in-house |
| LY2 FullORF N-His | LY2 | Ring2 | FullORF | 6xHis | N-ter | Yes | Yes | Yes | in-house |
| LY2 FullORF C-His | LY2 | Ring2 | FullORF | 6xHis | C-ter | Yes | Yes | Yes | in-house |
| LY2 ORF1 | LY2 | Ring2 | ORF1 | no-tag | NA | Yes | Yes | No | in-house |
| LY2 ORF1 N-His | LY2 | Ring2 | ORF1 | 6xHis | N-ter | Yes | Yes | Yes | in-house |
| LY2 ORF1 C-His | LY2 | Ring2 | ORF1 | 6xHis | C-ter | Yes | Yes | Yes | in-house |
| LY2 | LY2 | Ring2 | ORF1 (delta- | no-tag | NA | Yes | No | No | in-house |
| ORF1(dR) | | | arginine rich | | | | | | |
| | | | region) | | | | | | |
| LY2 | LY2 | Ring2 | ORF1 (delta- | 6xHis | N-ter | Yes | Yes | Yes | in-house |
| ORF1(dR) N-His | | | arginine rich | | | | | | |
| | | | region) | | | | | | |
| LY2 | LY2 | Ring2 | ORF1 (delta- | 6xHis | C-ter | Yes | Yes | Yes | in-house |
| ORF1(dR) C-His | | | arginine rich | | | | | | |
| | | | region) | | | | | | |
| LY2 ORF1/1 | LY2 | Ring2 | ORF1/1 | no-tag | NA | Yes | Yes | No | in-house |
| LY2 ORF1/1 N-His | LY2 | Ring2 | ORF1/1 | 6xHis | N-ter | Yes | Yes | Yes | in-house |
| LY2 ORF1/1 C-His | LY2 | Ring2 | ORF1/1 | 6xHis | C-ter | Yes | Yes | Yes | in-house |
| LY2 ORF1/2 | LY2 | Ring2 | ORF1/2 | no-tag | NA | Yes | Yes | No | in-house |
| LY2 ORF1/2 N-His | LY2 | Ring2 | ORF1/2 | 6xHis | N-ter | Yes | Yes | Yes | in-house |
| LY2 ORF1/2 C-His | LY2 | Ring2 | ORF1/2 | 6xHis | C-ter | Yes | Yes | Yes | in-house |
| LY2 ORF2 | LY2 | Ring2 | ORF2 | no-tag | NA | Yes | Yes | No | in-house |
| LY2 ORF2 N-His | LY2 | Ring2 | ORF2 | 6xHis | N-ter | Yes | Yes | Yes | in-house |
| LY2 ORF2 C-His | LY2 | Ring2 | ORF2 | 6xHis | C-ter | Yes | Yes | Yes | in-house |
| LY2 ORF2/2 | LY2 | Ring2 | ORF2/2 | no-tag | NA | Yes | Yes | No | in-house |
| LY2 ORF2/2 N-His | LY2 | Ring2 | ORF2/2 | 6xHis | N-ter | Yes | Yes | Yes | in-house |
| LY2 ORF2/2 C-His | LY2 | Ring2 | ORF2/2 | 6xHis | C-ter | Yes | Yes | Yes | in-house |
| LY2 ORF2/3 | LY2 | Ring2 | ORF2/3 | no-tag | NA | Yes | Yes | No | in-house |
| LY2 ORF2/3 N-His | LY2 | Ring2 | ORF2/3 | 6xHis | N-ter | Yes | Yes | Yes | in-house |
| LY2 ORF2/3 C-His | LY2 | Ring2 | ORF2/3 | 6xHis | C-ter | Yes | Yes | Yes | in-house |
| LY2 ORF2/ORF1 C- | LY2 | Ring2 | ORF2/ORF1 | 6xHis | C-ter | Yes | Yes | Yes | in-house |
| His | | | | | | | | | |
| LY2 ORF1 HisE354 | LY2 | Ring2 | ORF1 | 6xHis | After E354 | Yes | Yes | No | in-house |
| LY2 ORF1 HisN299 | LY2 | Ring2 | ORF1 | 6xHis | After N299 | Yes | Yes | No | in-house |
| LY2 ORF1 HisL267 | LY2 | Ring2 | ORF1 | 6xHis | After L267 | Yes | Yes | No | in-house |
| tth8 ORF1 (JA20 | tth8 | Ring1 | ORF1 (with | 6xHis | C-ter | Yes | No | No | in-house |
| HVR) | | | JA20's | | | | | | |
| | | | hypervariable | | | | | | |
| | | | region) | | | | | | |
| tth8 ORF1 | tth8 | Ring1 | ORF1 (with | 6xHis | C-ter | Yes | No | No | in-house |
| (TJN02 | | | TJN02's | | | | | | |
| HVR) | | | hypervariable | | | | | | |
| | | | region) | | | | | | |
| tth8 ORF1 (TTV16 | tth8 | Ring1 | ORF1 (with | 6xHis | C-ter | Yes | No | No | in-house |
| (HVR) | | | TTV16's | | | | | | |
| | | | hypervariable | | | | | | |
| | | | region) | | | | | | |
| Ring2 ORF1 (CodOpt) | LY2 | Ring2 | ORF1 (codon | no-tag | NA | Yes | Yes | Yes | Medigen |
| | | | optimized) | | | | | | |
| Ring2 ORF1 (CodOpt) | LY2 | Ring2 | ORF1 (codon | 6xHis | C-ter | Yes | Yes | Yes | Medigen |
| HRV3C-6His | | | optimized) | | | | | | |
| Ring4 ORF1 (CodOpt) | 6B.CD8. | Ring4 | ORF1 (codon | no-tag | NA | Yes | Yes | Yes | Medigen |
| | contig2 | | optimized) | | | | | | |
| RIng4 ORF1 (CodOpt) | 6B.CD8. | Ring4 | ORF1 (codon | 6xHis | C-ter | Yes | Yes | Yes | Medigen |
| HRV3C-6His | contig2 | | optimized) | | | | | | |
| RIng5.2 ORF1 | CT30F | Ring5.2 | ORF1 (codon | no-tag | NA | Yes | Yes | Yes | Medigen |
| (CodOpt) | | | optimized) | | | | | | |
| Ring5.2 ORF1 | CT30F | RIng5.2 | ORF1 (codon | 6xHis | C-ter | Yes | Yes | Yes | Medigen |
| (CodOpt) HRV3C- | | | optimized) | | | | | | |
| 6His | | | | | | | | | |
| Ring6 ORF1 (CodOpt) | 190783.3 | Ring6 | ORF1 (codon | no-tag | NA | Yes | Yes | Yes | Medigen |
| | | | optimized) | | | | | | |
| Ring6 ORF1 (CodOpt) | 190783.3 | Ring6 | ORF1 (codon | 6xHis | C-ter | Yes | Yes | Yes | Medigen |
| HRV3C-6His | | | optimized) | | | | | | |
| Ring1 ORF1 (CosOpt) | tth8 | Ring1 | ORF1 (codon | 6xHis | C-ter | Yes | Yes | Yes | Medigen |
| His | | | optimized) | | | | | | |
| Rig3.1 ORF1 | 6B.CD8. | Ring3.1 | ORF1 (codon | 6xHis | C-ter | Yes | Yes | Yes | Medigen |
| (CodOpt) His | contig3 | | optimized) | | | | | | |
| Ring7 ORF1 (CodOpt) | 190783.4 | Ring7 | ORF1 (codon | 6xHis | C-ter | Yes | Yes | Yes | Medigen |
| His | | | optimized) | | | | | | |
| Ring2 (CodOpt) N-His | LY2 | Ring2 | ORF1 (codon | 6xHis | N-ter | Yes | Yes | Yes | Medigen |
| | | | optimized) | | | | | | |
| Ring2 (CodOpt) N-His | LY2 | Ring2 | ORF1 (codon | 6xHis- | N-ter | Yes | Yes | Yes | Medigen |
| (PS) | | | optimized) | PreScision | | | | | |
| | | | | Protease | | | | | |
| | | | | recognition | | | | | |
| | | | | sequence) | | | | | |
| Ring2 tandem | LY2 | Ring2 | 2x whole | no-tag | NA | Yes | Yes | Yes | Medigen |
| | | | genome | | | | | | |
| | | | (without | | | | | | |
| | | | Polyhedrin | | | | | | |
| | | | promoter) | | | | | | |
| WTLY2 | LY2 | Ring2 | whole genome | no-tag | NA | Yes | Yes | Yes | in-house |
| WTtth8 | tth8 | Ring1 | whole genome | no-tag | NA | Yes | Yes | Yes | in-house |
| WTtth8 (Reverse) | tth8 | Ring1 | whole genome | no-tag | NA | Yes | Yes | Yes | in-house |
| | | | (with | | | | | | |
| | | | Reversed 5′ | | | | | | |
| | | | Polyhedrin | | | | | | |
| | | | promoter) | | | | | | |
| LoxPWTLY2 | LY2 | Ring2 | LoxP-whole | no-tag | NA | Yes | Yes | Yes | in-house |
| | | | genome-LoxP | | | | | | |
| Cre-R | NA | NA | Cre | no-tag | NA | Yes | Yes | Yes | in-house |
| | | | recombinase | | | | | | |

“FullORF” = Full ORF-containing region, with noncoding regions removed; ORF2/3 tagged.

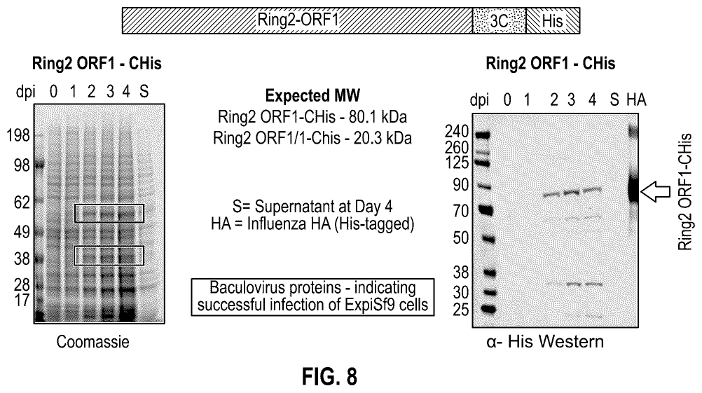

On the day before infection, ExpiSf9 cells were seeded at 5×10⁶ cells/mi in 25 ml room temperature ExpiSf9 CD Medium in 125 ml Nalgene Single-Use PETG Erlenmeyer Plain Bottom Flask [iThermofisher Scientific Catalog no: 4115-0125]. The cell viability was monitored to ensure that it was maintained at or above 95%, 100 μL of ExpiSf Enhancer solution was added to the cells in a dropwise manner. Cells were incubated with shaking overnight in a 27° C. non-humidified, air-regulated, non-CO₂ atmosphere incubator using an orbital shaker at 125±5 rpm. On Day 1, approximately 18-24 hours after adding ExpiSf Enhancer, cells were infected with the indicated baculovirus at a multiplicity of infection (MOI) of 5 and incubated under the same conditions. Cells were harvested 72 hours post infection and viability was found to range between 60 and 80%. To analyze samples, cells were lysed by adding 1× Bolt LDS sample buffer [Invitrogen Catalog No.: B0007]and 1× Bolt reducing agent [Invitrogen Catalog No.: B0009]and sonicating for 2.5 minutes. As shown in FIG. 8, C-His-tagged LY2 ORF1 was successfully expressed in infected ExpiSf9 cells by day 2 post-infection as determined by western blotting using an anti-poly-histidine antibody. In addition, baculovirus proteins were detected by Coomassie staining, indicating a successful infection.

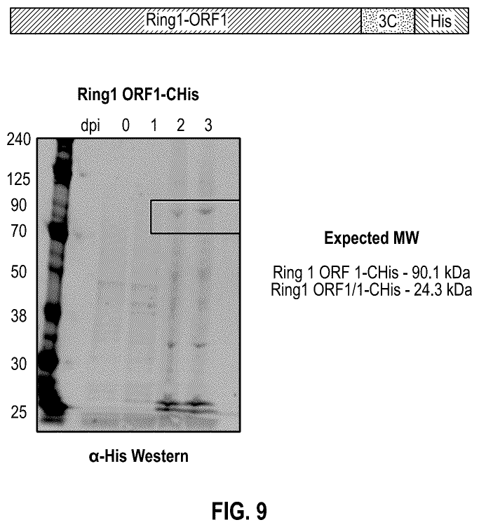

As shown in FIG. 9, C-His-tagged tth8 ORF1 and ORF1/1 were also successfully expressed in infected ExpiSf9 cells by day 2 post-infection.

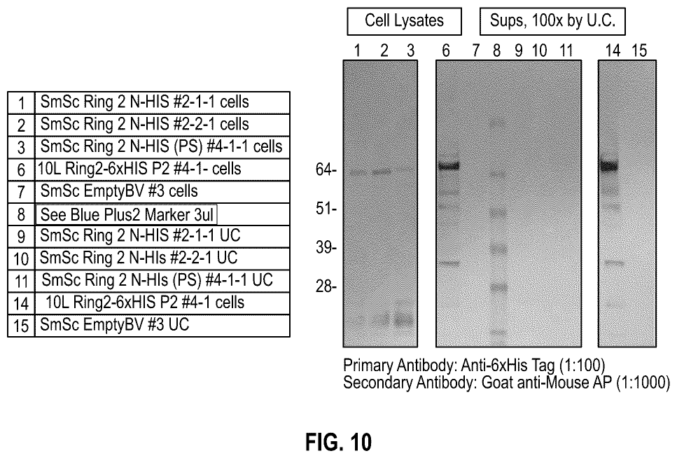

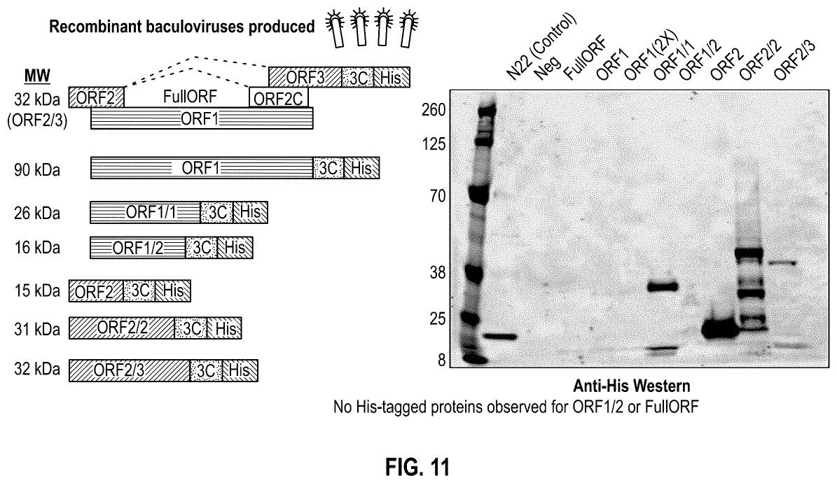

N-terminally His-tagged LY2 ORF1 expression was also detected in infected ExpiSf9 cells (FIG. 10). Here, constructs either comprised an N-terminal His-tag which was immediately followed by the wildtype ORF1 sequence (lanes 1, 2, 9, 10, or 14), or an N-terminal His-tag which was followed by a rhinovirus 3C cleavage sequence (lanes 3, 11). Samples in lanes 1 to 7 are lysates loaded directly onto the gel, whereas samples in lanes 9-15 were prepared by first pelleting protein from conditioned medium via ultracentrifugation and resuspending the pellet in a 100-fold smaller volume. Samples shown in lanes 1-3 and 9-11 were grown at a small (5 mL) scale. Samples in lanes 6 and 14 were obtained from a 1OL culture. Thus, this example shows that production of ORF1 from a plurality of strains with N or C terminal poly-histidine tags can be successfully carried out at a scale ranging from 5 mL to 10L, and that ORF1 can be found in Sf9 lysate or culture supernatant (conditioned medium).

### Example 15: Expression of Ring1 ORFs in Sf9 cells

In this example, a series of recombinant baculoviruses were produced with alternate arrangements of tth8 ORFs, each tagged with a C-terminal poly-histidine (FIG. 11). The recombinant baculovirus designs included one baculovirus construct for each of the tth8 ORF splice variants (i.e., ORF1, ORF1/1, ORF1/2, ORF2, ORF2/2, and ORF2/3), as well as a “Ful10RF” construct containing the full ORF region from tth8, driven by the baculovirus polyhedrin promoter. These baculoviruses were produced as described in Example 14.

Protein expression was then detected by western blot using anti-poly-histidine antibody. As shown in FIG. 11, His-tagged tth8 ORFs ORF1/1, ORF1/2, ORF2, ORF2/2 and ORF2/3 were detected.

### Example 16: Expression of Ring2 ORFs in Sf9 Cells

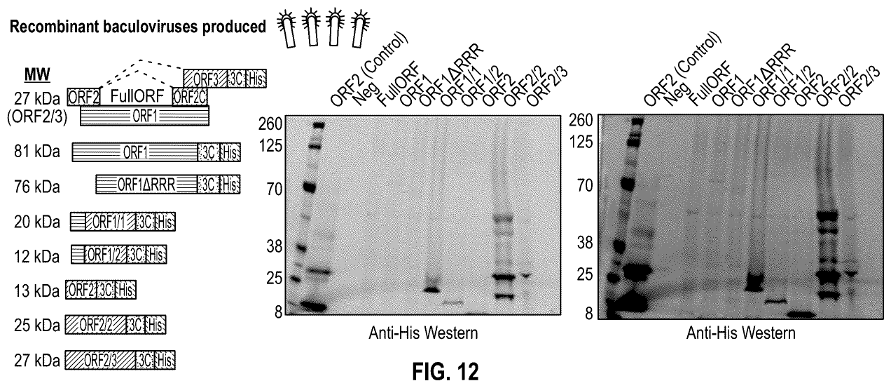

In one example, a series of recombinant baculoviruses were produced with alternate arrangements of Ring2 ORFs, each tagged with a poly-histidine tag at the C terminus (FIG. 12). The recombinant baculovirus designs included one baculovirus construct for each of the Ring2 ORF splice variants (i.e., ORF1, ORF1/1, ORF1/2, ORF2, ORF2/2, and ORF2/3), a variant in which the N-terminal arginine-rich region (RRR) is deleted (ORF1ARRR), as well as a “FullORF” construct containing the full ORF region from Ring2 driven by the baculovirus polyhedrin promoter. For each experimental condition, ExpiSf9 cells were infected with recombinant baculoviruses expressing individual Ring2 variants at an MOI of 5. The experimental conditions for this were as described in Examples 14 and 15.

Protein expression was then detected by western blot using anti-His. As shown in FIG. 12, His-tagged Ring2 ORFs ORF1, ORF1ARRR, ORF1/1, ORF1/2, ORF2, ORF2/2, and ORF2/3 were all detected.

In a further experiment as part of this example, recombinant baculoviruses comprising a Ring2 ORF1-encoding sequence and/or a Ring2 ORF2 splice variant-encoding sequence were used to infect Sf9 cells. The expression conditions tested included ORF1 alone, or co-infection of ORF1+“FullORF”, ORF1+ORF2, ORF1+ORF2/2, and ORF1+ORF2/3, as well as a negative control labeled ‘Neg’. ExpiSf9 cells were co-infected with baculoviruses at a MOI of 5 for each condition. Experimental conditions were as described in Examples 14 and 15. Protein expression of ORF1, ORF2, ORF2/2, and ORF2/3 was then assessed for each condition by western blot using either anti-His or anti-Ring2 N22. The latter is a monoclonal antibody that was obtained by immunizing mice with the N22 fragment of Ring2 ORF1 produced in E. coli, and then generating hybridomas.

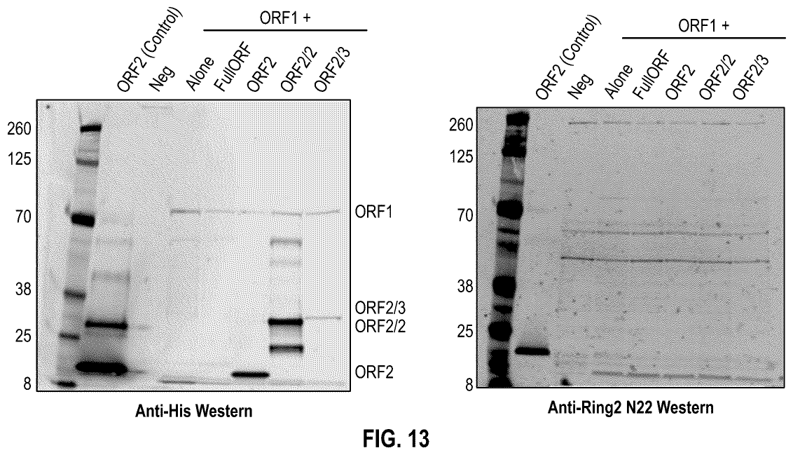

As shown in FIG. 13, both Westerns detected ORF1 as a band at ˜81kD in each of the ORF1-infected conditions. The ORF1 band is highlighted by a dashed box in the anti-N22 Western, and is not visible in the negative control (Neg) sample. The lower molecular weight (˜10 kD) band detected by both antibodies is thought to be a C-terminal fragment of ORF1. ORF2, ORF2/2, and ORF2/3 were also detected in the corresponding samples (anti-His blot). Thus, this example illustrates that both ORF1 and individual splice variants of ORF2 can be co-expressed in insect cells.

### Example 17: Expression of all Ring2 ORFs Simultaneously in Sf9 Cells

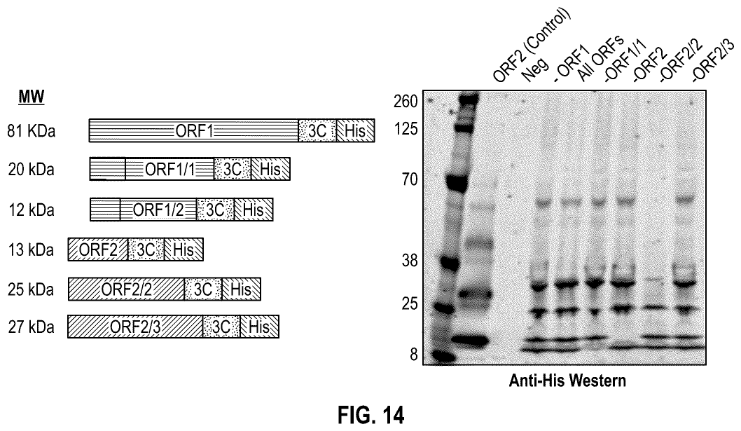

In one example, a series of six recombinant baculoviruses were produced, each designed to express a particular Ring2 ORF (i.e., ORF1, ORF1/1, ORF1/2, ORF2, ORF2/2, and ORF2/3), each tagged with a His tag (FIG. 14), as described in Example 16. Sf9 cells were infected with various combinations of the Ring2 ORF baculoviruses —specifically, each condition involved infecting cells with all but one ORF construct, as indicated in FIG. 14. Protein expression was then detected by western blot of whole cell suspension using anti-His. As shown in FIG. 14, His-tagged Ring2 ORFs were detected in the expected pattern. Either all ORFs were detected, or all except for the omitted one.

### Example 18: Co-Delivery and Independent Expression of Anellovirus Genomes and Recombinant Anellovirus ORFs in Sf9 Cells

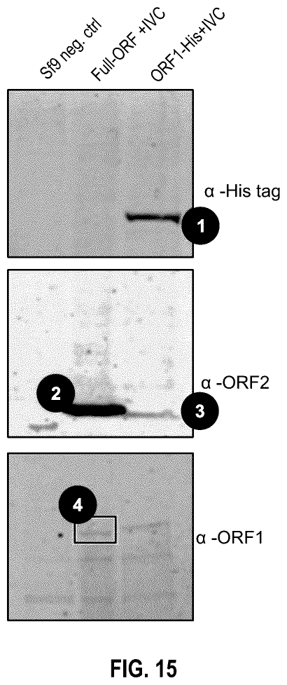

In this example, anellovirus ORFs and genomes were co-delivered in Sf9 cells by transfecting an in vitro circularized (IVC) anellovirus genome and infecting the cells with baculovirus encoding ORF1 tagged at its C-terminus with hexa-histidine (FIG. 15). Protein expression was then detected by western blot using anti-His, anti-ORF2, and anti-ORF1 monoclonal antibody targeting the N22 fragment. As shown (FIG. 15, bullet 1), His-tagged ORF1 was detected in this preparation showing successful recombinant ORF1 expression from the baculovirus vector. Consistent with this result, the same ORF1 protein was detected using the anti-ORF1 antibody (FIG. 15, bottom panel, right-most lane).

In the same sample of treated cells, the native anellovirus promoter was shown to be transcriptionally active in Sf9 cells because ORF2 expression was detected (FIG. 15, bullet 3) and could only have been produced by the IVC genome which was transfected into the cells.

In addition, Anellovirus ORFs were co-delivered and expressed in Sf9 cells using an in vitro circularized (IVC) construct and a FullORF baculovirus. Protein expression was then detected by western blot using anti-His, anti-Ring2 ORF2, and anti-Ring2 ORF1 N22. ORF1 protein was detected in the cells (FIG. 15, bullet 4) and could be the product of either the IVC or the FullORF baculovirus construct. Surprisingly, ORF2 protein was readily detected and its intensity suggests the expression is derived from the FullORF baculovirus construct (FIG. 15, bullet 2).

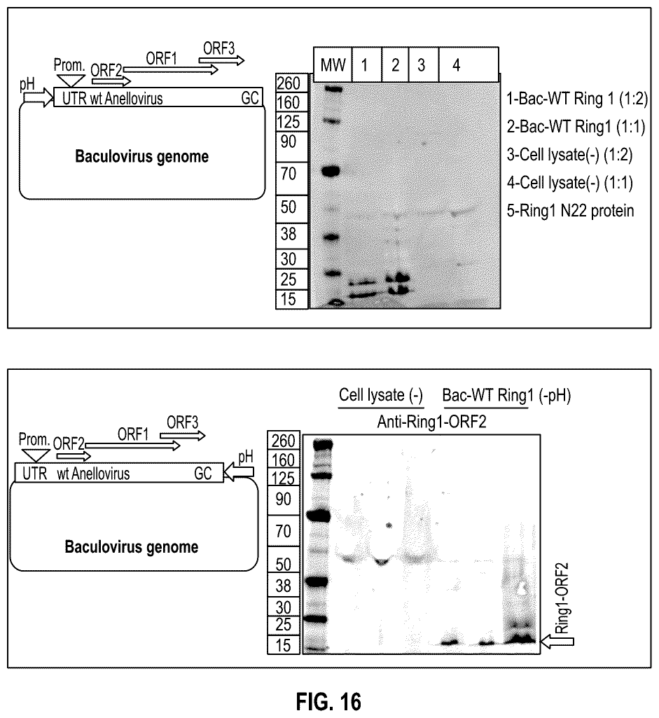

As a further test of the ability of the anellovirus genome to express its genes in insect cells, the tth8 anellovirus coding region was cloned into the pFastBac vector in both orientations. This yielded ‘FullORF’ tth8 baculovirus constructs in which the polyhedrin promoter was positioned upstream of either the sense or the anti-sense direction of the coding region. The latter configuration is highly unlikely to initiate transcription of the anellovirus genes. Consistent with our surprising observations in Ring2, expression of tth8 ORF2 was independent of the orientation of the coding region relative to the baculovirus polyhedrin promoter, suggesting that expression is driven by the anellovirus promoter (FIG. 16, bands at ˜15 and 20 kDa).

This example shows that IVC transfections and baculovirus infections can co-deliver functional anellovirus genes to Sf9 insect cells and that the native anellovirus promoter is active in these cells.

### Example 19: Anellovirus ORF1 Associates with DNA in Sf9 Cells to Form Complexes Isolated by Isopycnic Centrifugation

In this example, Sf9 cells were transfected with IVC anellovirus genome LY2, infected with a baculovirus encoding LY2 ORF1 with a C-terminal poly-histidine tag, and then fractionated to determine whether ORF1 expressed using the baculoviral expression system forms protein-DNA complexes that can be isolated in vitro.

CsCl gradients were prepared by adding 8 ml of 1.2 g/ml CsCl solution (in TN buffer; 20 mM Tris pH 8.0, 140 mM NaCl) to ultracentrifuge tubes (Ultra-Clear 17 ml—Beckman #344061) for SW32.1 Ti rotor. Tubes were underlayed with 8 ml of 40% CsCl (in TN buffer), then capped with topper and run on Gradient Master program 5-50% for 13 minutes to prepare linear gradient. The caps were removed and the gradients overlayed with 0.5 ml-2 ml of Sf9 lysate to each tube and topped off to near the top with TN buffer containing 0.001% Poloxamer-188. Ultracentrifugation was for 18.5 hours at 22,500×RPM. Fractions were collected from the gradient by piercing the bottom of the tube and allowing ˜600 ul fractions to flow into wells of a deep well block. The refractive index of each sample was measured to determine their density.

Anelloviral DNA content in the fractions was then determined by first extracting DNA from the fractions, and then by carrying out qPCR. Pure Link Viral DNA extraction Kit [Thermofisher Scientific Catalog no. 12280050]was used to purify viral DNA from 50 μL of the fractions. The samples were treated with Proteinase K and lysed using Lysis buffer by incubating at 56° C. for 15 min., washed with 99% ethanol, and transferred to a Viral Spin Column. Samples were centrifuged at 6800×g, washed twice with 500 uL Wash buffer provided with the kit and centrifuged again. 100 μL of RNase-free water was added to the column to elute the DNA.

For qPCR, 2× TaqMan Gene Expression Master Mix, 100 uM LY2 primers Forward (AGCAACAGGTAATGGAGGAC), 100 uM LY2 Reverse (TGAAGCTGGGGTCTTTAAC) along with 100 uM LY2 Probe (TCTACCTAGGTGCAAAGGGCC) were diluted in 5.83 uL Nuclease Free water for each reaction. The following conditions were used for each qPCR cycle: 50 -C hold for 2 minutes, 95 -C hold for 10 minutes followed by 40 cycles of 95° C. for 15 seconds and 60° C. for 1 minute on an Applied Biosystems Quant Studio 3 Real-Time PCR Machine. Each sample was run in triplicate and the entire assay was repeated thrice and used to plot the graph.

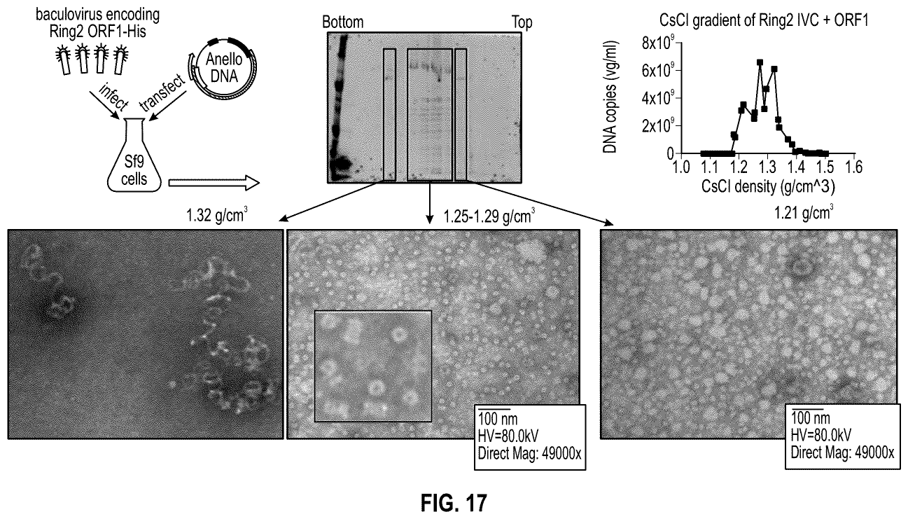

As shown in FIG. 17, isopycnic fractions were characterized by western blotting, quantitative PCR, and transmission electron microscopy. Anti-his western blotting of gradient fractions showed clear bands of the expected molecular weight for LY2 ORF1 in fractions having densities of 1.32 g/mL and 1.21 g/mL. In addition, fractions ranging from 1.25 to 1.29 g/mL had clear bands of higher and lower molecular weights than expected. Also, qPCR indicates the presence of LY2 genomic DNA in certain fractions, with peaks at approximately 1.21 g/mL, 1.29 g/mL, and 1.32 g/mL.

Negative stain transmission electron microscopy was carried out on the 1.32 g/mL and 1.21 g/mL fractions, as well as a pool of fractions ranging from 1.25 to 1.29 g/mL. The pool shows an abundance of particles, including several having the appearance of proteasomes. The presence of proteasomes may explain the western blot bands at low and high molecular weights. The former may be due to proteolytic degradation and the latter due to ubiquitylated ORF1, or ORF1 fragments covalently associated with proteasome proteins in the course of degradation. The 1.21 g/mL fraction shows particles of various sizes, including several which appear to be consistent with lipid-based particles. The 1.32 g/mL fraction shows remarkable DNA-like structures that stain differently than naked DNA, suggesting association with macromolecules such as protein.

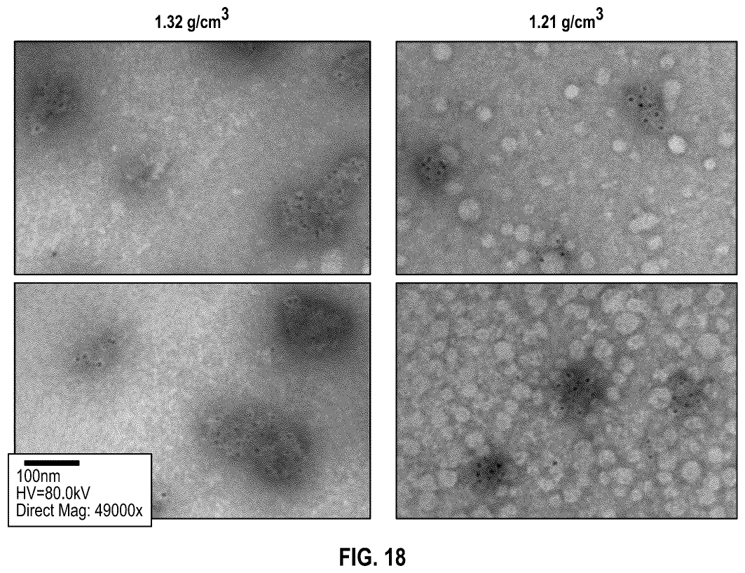

To determine if LY2 ORF1 is associated with the structures observed in the electron micrographs, immunogold detection using an anti-poly-histidine antibody was carried out. FIG. 18 shows gold label accumulating on the structures observed in the 1.32 and 1.21 g/mL fractions, consistent with the presence of ORF1-His in association with the DNA seen in the 1.32 g/mL fraction, and in the particles seen in the 1.21 g/mL fraction.

Taken together, these results show that ORF1 expressed in Sf9 cells can associate with DNA to form complexes having a density consistent with anellovirus particles.

### Example 20: Expression of ORF1 Protein from a Diverse Array of Anelloviruses Using Baculovirus

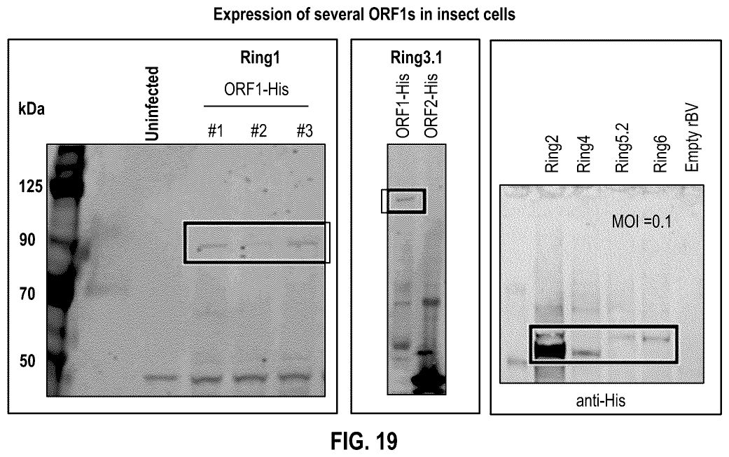

In this example, Sf9 cells were infected with baculoviruses engineered to express C-terminal His-tagged ORF1 proteins from anellovirus strains Ring3.1, Ring4, Ring5.2, Ring6, as well as Ring1 and Ring2. As shown in FIG. 19, ORF1 protein originating from each of the Anellovirus strains were successfully expressed in Sf9 cells. As shown in Table E3, Anellovirus ORF1 from the strains representing all three genera (Alphatorquevirus, Betatorquevirus, and Gammatorquevirus), were tested and their expression level is seen in FIGS. 3, 4, 5, and 19. In general, we find that the level of expression in this system is highest for ORF1 from Betatorqueviruses, intermediate from Gammatorqueviruses, and lowest from Alphatorqueviruses.

### Example 21: In Vitro Assembly of Anellovectors Using Components Produced Via Baculovirus System

In this example, baculovirus constructs suitable for expression of Anellovirus proteins (e.g., ORF1) are generated by in vitro assembly.

DNA encoding Anellovirus ORF1 (wildtype protein, chimeric protein or fragments thereof) which may be untagged or contain tags fused N-terminally, C-terminally, or harbor mutations within the ORF1 protein itself to introduce a tag to aid in purification and/or identity determination through immunostaining assays (such as, but not limited to, ELISA or Western Blot) is expressed in insect cell lines (Sf9 and/or HighFive). Anellovirus ORF1 may be expressed alone or in combination with any number of helper proteins including, but not limited to, Anellovirus ORF2 and/or ORF3 proteins.

Protein is purified using developed purification techniques potentially including but not limited to chelating purification, heparin purification, gradient sedimentation purification and/or size exclusion purification. ORF1 is evaluated for its ability to form capsomers or VLPs and used in subsequent steps for nucleic acid encapsidation.

In one example, DNA encoding Ring2 ORF1 fused to an N-terminal HIS₆-tag (HIS-ORF1) was codon optimized for insect expression and cloned into the baculovirus expression vector pFASTbac system to generate a baculovirus expressing Ring2 ORF-HIS recombinant protein using the Bac-to-BAC expression system according to manufacturer's method (ThermoFisher Scientific). 10 liters of insect cells (Sf9) were infected with Ring2 HIS-ORF1 baculovirus and the cells were harvested 3-days post-infection by centrifugation. The cells were lysed, and the lysate was purified using a chelating resin column using standard art in the field. The elution fraction containing HIS-ORF1 was dialyzed and treated with DNAse to digest host cell DNA. The resulting material was purified again using a chelating resin column and fractions containing ORF1 were retained for nucleic acid encapsidation and viral vector purification.

Nucleic acid encapsidation and viral vector purification: Ring ORF1 (wildtype protein, chimeric protein or fragments thereof) is treated with conditions sufficient to dissociate VLPs or viral capsids to enable reassembly with nucleic acid cargo. Nucleic acid cargo can be defined as double stranded DNA, single stranded DNA, or RNA which encodes a gene of interest that one wants to deliver as a therapeutic agent. Potential conditions sufficient to dissociate VLPs or viral capsids can, but are not limited to, buffers of different pH, conditions of defined conductivity (salt content), conditions containing detergents (such as SDS, Tween, Triton), conditions containing chaotropic agents (such as Urea) or conditions involving defined temperature and time (reannealing temperatures). Nucleic acid cargo of defined concentration is combined with Ring ORF1 of defined concentration and treated with conditions sufficient to permit nucleic acid encapsidation. The resulting particle, defined as viral vector, is subsequently purified, e.g., using developed standard viral purification procedures.

In one example, single stranded circular DNA of a GFP-expression plasmid is added to a solution of Ring 2 HIS-ORF1 and the resulting sample is treated with 0.1% SDS in 50 mM Tris pH 8 buffer at 37C for 30 minutes. The resulting solution is further purified using a heparin column and the viral vector eluted from the column using a gradient of increasing NaCl concentration. The integrity of the viral vector is tested by transducing the cell lines EKVX and HEK293, and observing GFP production in at least one of the cell lines by fluorescence microscopy, demonstrating encapsidation of the nucleic acid cargo by the ORF1 protein to form the viral vector.

TABLE E3
Strains for which recombinant ORF1 expression was successful

| Name | Genus |
|---|---|
| Ring1 | Alpha |
| Ring2 | Beta |
| Ring3.1 | Gamma |
| Ring4 | Gamma |
| Ring5.2 | Alpha |
| Ring6 | Alpha |
| HLH | Beta |
| ctgh3 | Beta |
| LY1 | Beta |

### Example 22: Identification and Use of Protein Binding Sequences

This example describes putative protein-binding sites in the Anellovirus genome, which can be used for amplifying and packaging effectors, e.g., in an anellovector as described herein. In some instances, the protein-binding sites may be capable of binding to an exterior protein, such as a capsid protein.

Two conserved domains within the Anellovirus genome are putative origins of replication: the 5′ UTR conserved domain (5CD) and the GC-rich domain (GCR) (de Villiers et al., Journal of Virology 2011; Okamoto et al., Virology 1999). In one example, in order to confirm whether these sequences act as DNA replication sites or as capsid packaging signals, deletions of each region are made in plasmids harboring an Anellovirus sequence. A539 cells are transfected with the deletion constructs. Transfected cells are incubated for four days, and then virus is isolated from supernatant and cell pellets. A549 cells are infected with virus, and after four days, virus is isolated from the supernatant and infected cell pellets. qPCR is performed to quantify viral genomes from the samples. Disruption of an origin of replication prevents viral replicase from amplifying viral DNA and results in reduced viral genomes isolated from transfected cell pellets compared to wild-type virus. A small amount of virus is still packaged and can be found in the transfected supernatant and infected cell pellets. In some embodiments, disruption of a packaging signal will prevent the viral DNA from being encapsulated by capsid proteins. Therefore, in embodiments, there will still be an amplification of viral genomes in the transfected cells, but no viral genomes are found in the supernatant or infected cell pellets.

In a further example, in order to characterize additional replication or packaging signals in the DNA, a series of deletions across the entire TTMV-LY2 genome is used. Deletions of 100 bp are made stepwise across the length of the sequence. Plasmids harboring Anellovirus genome deletions are transfected into A549 and tested as described above. In some embodiments, deletions that disrupt viral amplification or packaging will contain potential cis-regulatory domains.

Replication and packaging signals can be incorporated into effector-encoding DNA sequences (e.g., in a genetic element in an anellovector) to induce amplification and encapsulation. This is done both in context of larger regions of the anellovector genome (i.e., inserting effectors into a specific site in the genome, or replacing viral ORFs with effectors, etc.), or by incorporating minimal cis signals into the effector DNA. In cases where the anellovector lacks trans replication or packaging factors (e.g., replicase and capsid proteins, etc.), the trans factors are supplied by helper genes. The helper genes express all of the proteins and RNAs sufficient to induce amplification and packaging, but lack their own packaging signals. The anellovector DNA is co-transfected with helper genes, resulting in amplification and packaging of the effector but not of the helper genes.

### Example 23: Replication-Deficient Anellovectors and Helper Viruses

For replication and packaging of an anellovector, some elements (e.g., an ORF1, ORF1/1, ORF1/2, ORF2, ORF2/2, ORF2/3, and/or ORF2t/3 molecule, or a nucleic acid sequence encoding same) can be provided in trans. These include proteins or non-coding RNAs that direct or support DNA replication or packaging. Trans elements can, in some instances, be provided from a source alternative to the anellovector, such as a helper virus, plasmid, or from the cellular genome.

Other elements are typically provided in cis (e.g., a TATA box, cap site, initiator element, transcriptional start site, 5′ UTR conserved domain, three open-reading frame region, poly(A) signal, or GC-rich region). These elements can be, for example, sequences or structures in the anellovector DNA that act as origins of replication (e.g., to allow amplification of anellovector DNA) or packaging signals (e.g., to bind to proteins to load the genome into the capsid). Generally, a replication deficient virus or anellovector will be missing one or more of these elements, such that the DNA is unable to be packaged into an infectious virion or anellovector even if other elements are provided in trans.

Replication deficient viruses can be useful for controlling replication of an anellovector (e.g., a replication-deficient or packaging-deficient anellovector) in the same cell. In some instances, the virus will lack cis replication or packaging elements, but express trans elements such as proteins and non-coding RNAs. Generally, the therapeutic anellovector would lack some or all of these trans elements and would therefore be unable to replicate on its own, but would retain the cis elements. When co-transfected/infected into cells, the replication-deficient virus would drive the amplification and packaging of the anellovector. The packaged particles collected would thus be comprised solely of therapeutic anellovector, without contamination from the replication-deficient virus.

To develop a replication deficient anellovector, conserved elements in the non-coding regions of Anellovirus will be removed. In particular, deletions of the conserved 5′ UTR domain and the GC-rich domain will be tested, both separately and together. Both elements are contemplated to be important for viral replication or packaging. Additionally, deletion series will be performed across the entire non-coding region to identify previously unknown regions of interest.

Successful deletion of a replication element will result in reduction of anellovector DNA amplification within the cell, e.g., as measured by qPCR, but will support some infectious anellovector production, e.g., as monitored by assays on infected cells that can include any or all of qPCR, western blots, fluorescence assays, or luminescence assays. Successful deletion of a packaging element will not disrupt anellovector DNA amplification, so an increase in anellovector DNA will be observed in transfected cells by qPCR. However, the anellovector genomes will not be encapsulated, so no infectious anellovector production will be observed.

### Example 24: Manufacturing process for replication-competent anellovectors

This example describes a method for recovery and scaling up of production of replication-competent anellovectors. Anellovectors are replication competent when they encode in their genome all the required nucleic acid elements and ORFs necessary to replicate in cells. Since these anellovectors are not defective in their replication they do not need a complementing activity provided in trans. They might, however need helper activity, such as enhancers of transcriptions (e.g. sodium butyrate) or viral transcription factors (e.g. adenoviral E1, E2 E4, VA; HSV Vp16 and immediate early proteins).

In this example, double-stranded DNA encoding the full sequence of a synthetic anellovector either in its linear or circular form is introduced into 5E+05 adherent mammalian cells in a T75 flask by chemical transfection or into 5E+05 cells in suspension by electroporation. After an optimal period of time (e.g., 3-7 days post transfection), cells and supernatant are collected by scraping cells into the supernatant medium. A mild detergent, such as a biliary salt, is added to a final concentration of 0.5% and incubated at 37° C. for 30 minutes. Calcium and Magnesium Chloride is added to a final concentration of 0.5 mM and 2.5 mM, respectively. Endonuclease (e.g. DNAse I, Benzonase), is added and incubated at 25-37° C. for 0.5-4 hours. Anellovector suspension is centrifuged at 1000×g for 10 minutes at 4° C. The clarified supernatant is transferred to a new tube and diluted 1:1 with a cryoprotectant buffer (also known as stabilization buffer) and stored at −80° C. if desired. This produces passage 0 of the anellovector (P0). To bring the concentration of detergent below the safe limit to be used on cultured cells, this inoculum is diluted at least 100-fold or more in serum-free media (SFM) depending on the anellovector titer.

A fresh monolayer of mammalian cells in a T225 flask is overlaid with the minimum volume sufficient to cover the culture surface and incubated for 90 minutes at 37° C. and 5% carbon dioxide with gentle rocking. The mammalian cells used for this step may or may not be the same type of cells as used for the P0 recovery. After this incubation, the inoculum is replaced with 40 ml of serum-free, animal origin-free culture medium. Cells are incubated at 37° C. and 5% carbon dioxide for 3-7 days. 4 ml of a 10× solution of the same mild detergent previously utilized is added to achieve a final detergent concentration of 0.5%, and the mixture is then incubated at 37° C. for 30 minutes with gentle agitation. Endonuclease is added and incubated at 25-37° C. for 0.5-4 hours. The medium is then collected and centrifuged at 1000×g at 4° C. for 10 minutes. The clarified supernatant is mixed with 40 ml of stabilization buffer and stored at −80° C. This generates a seed stock, or passage 1 of anellovector (P1).

Depending on the titer of the stock, it is diluted no less than 100-fold in SFM and added to cells grown on multilayer flasks of the required size. Multiplicity of infection (MOI) and time of incubation is optimized at smaller scale to ensure maximal anellovector production. After harvest, anellovectors may then be purified and concentrated as needed. A schematic showing a workflow, e.g., as described in this example, is provided in FIG. 20.

### Example 25: Manufacturing process of replication-deficient anellovectors

This example describes a method for recovery and scaling up of production of replication-deficient anellovectors.

Anellovectors can be rendered replication-deficient by deletion of one or more ORFs (e.g., ORF1, ORF1/1, ORF1/2, ORF2, ORF2/2, ORF2/3, and/or ORF2t/3) involved in replication. Replication-deficient anellovectors can be grown in a complementing cell line. Such cell line constitutively expresses components that promote anellovector growth but that are missing or nonfunctional in the genome of the anellovector.

In one example, the sequence(s) of any ORF(s) involved in anellovector propagation are cloned into a lentiviral expression system suitable for the generation of stable cell lines that encode a selection marker, and lentiviral vector is generated as described herein. A mammalian cell line capable of supporting anellovector propagation is infected with this lentiviral vector and subjected to selective pressure by the selection marker (e.g., puromycin or any other antibiotic) to select for cell populations that have stably integrated the cloned ORFs. Once this cell line is characterized and certified to complement the defect in the engineered anellovector, and hence to support growth and propagation of such anellovectors, it is expanded and banked in cryogenic storage. During expansion and maintenance of these cells, the selection antibiotic is added to the culture medium to maintain the selective pressure. Once anellovectors are introduced into these cells, the selection antibiotic may be withheld.

Once this cell line is established, growth and production of replication-deficient anellovectors is carried out, e.g., as described herein.

### Example 26: Production of anellovectors using suspension cells

This example describes the production of anellovectors in cells in suspension.

In this example, an A549 or 293T producer cell line that is adapted to grow in suspension conditions is grown in animal component-free and antibiotic-free suspension medium (Thermo Fisher Scientific) in WAVE bioreactor bags at 37 degrees and 5% carbon dioxide. These cells, seeded at 1×10⁶ viable cells/mL, are transfected using lipofectamine 2000 (Thermo Fisher Scientific) under current good manufacturing practices (cGMP), with a plasmid comprising anellovector sequences, along with any complementing plasmids suitable or required to package the anellovector (e.g., in the case of a replication-deficient anellovector, e.g., as described herein). The complementing plasmids can, in some instances, encode for viral proteins that have been deleted from the anellovector genome (e.g., an anellovector genome based on a viral genoe, e.g., an Anellovirus genome, e.g., as described herein) but are useful or required for replication and packaging of the anellovectors. Transfected cells are grown in the WAVE bioreactor bags and the supernatant is harvested at the following time points: 48, 72, and 96 hours post transfection. The supernatant is separated from the cell pellets for each sample using centrifugation. The packaged anellovector particles are then purified from the harvested supernatant and the lysed cell pellets using ion exchange chromatography.

The genome equivalents in the purified prep of the anellovectors can be determined, for example, by using a small aliquot of the purified prep to harvest the anellovector genome using a viral genome extraction kit (Qiagen), followed by qPCR using primers and probes targeted towards the anellovector DNA sequence, e.g., as described in Example 32.

The infectivity of the anellovectors in the purified prep can be quantified by making serial dilutions of the purified prep to infect new A549 cells. These cells are harvested 72 hours post transfection, followed by a qPCR assay on the genomic DNA using primers and probes that are specific to the anellovector DNA sequence.

### Example 27: Quantification of anellovector genome equivalents by qPCR

This example demonstrates the development of a hydrolysis probe-based quantitative PCR assay to quantify anellovectors. Sets of primers and probes are designed based on an Anellovirus genome sequence. using the software Geneious with a final user optimization. Exemplary primer sequences for TTV (Accession No. AJ620231.1) and TTMV (Accession No. JX134045.1) are shown in Table E4 below.

TABLE E4
Sequences of forward and reverse primers and
hydrolysis probes used to quantify TTMV and
TTV genome equivalents by quantitative PCR.

| | | SEQ |
|---|---|---|
| | | ID |
| TTMV | | NO: |
| Forward Primer | 5′-GAAGCCCACCAAAAGCAATT-3′ | 697 |
| Reverse Primer | 5′-AGTTCCCGTGTCTATAGTCGA-3′ | 698 |
| Probe | 5′-ACTTCGTTACAGAGTCCAGGGG-3′ | 699 |
| TTV | | |
| Forward Primer | 5′-AGCAACAGGTAATGGAGGAC-3′ | 700 |
| Reverse Primer | 5′-TGGAAGCTGGGGTCTTTAAC-3′ | 701 |
| Probe | 5′-TCTACCTTAGGTGCAAAGGGCC-3′ | 702 |

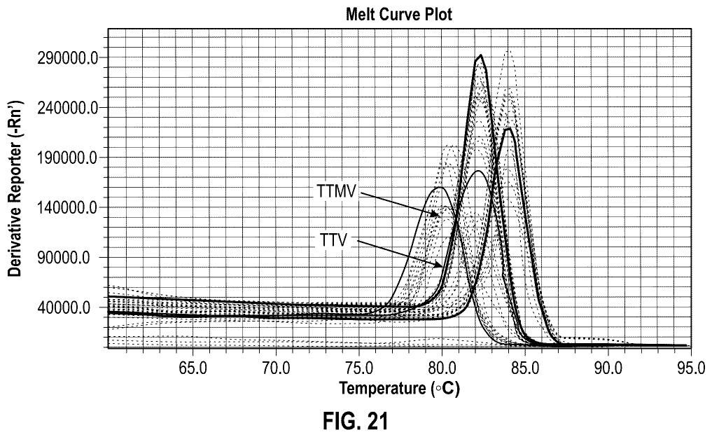

As a first step in the development process, qPCR is run using the Anellovirus primers with SYBR-green chemistry to check for primer specificity. FIG. 21 shows one distinct amplification peak for each primer pair.

Hydrolysis probes are ordered labeled with the fluorophore 6FAM at the 5′ end and a minor groove binding, non-fluorescent quencher (MGBNFQ) at the 3′ end. The PCR efficiency of the new primers and probes was evaluated using two different commercial master mixes using purified plasmid DNA as component of a standard curve and increasing concentrations of primers. The standard curve is set up by using purified plasmids containing the target sequences for the different sets of primers-probes.

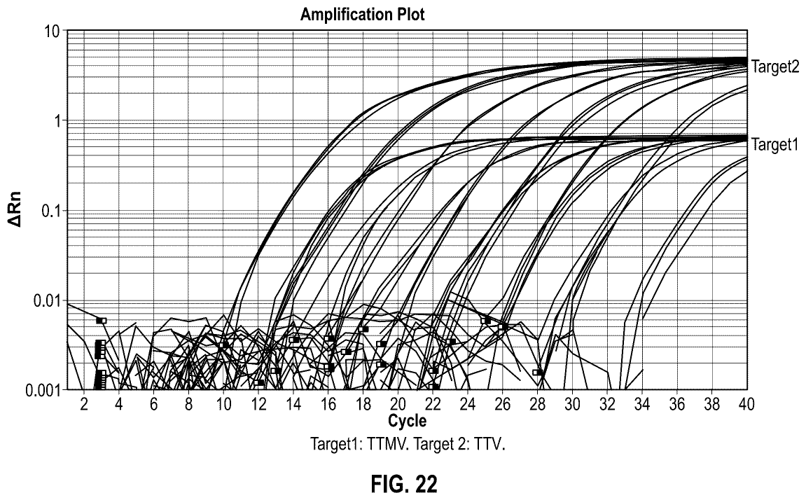

Seven tenfold serial dilutions are performed to achieve a linear range over 7 logs and a lower limit of quantification of 15 copies per 20 ul reaction. All primers for qPCR are ordered from a commercial vendor such as IDT. Hydrolysis probes conjugated to the fluorophore 6FAM and a minor groove binding, non-fluorescent quencher (MGBNFQ) as well as all the qPCR master mixes are obtained from Thermo Fisher. An exemplary amplification plot is shown in FIG. 22.

Using these primer-probe sets and reagents, the genome equivalent (GEq)/ml in anellovector stocks is quantified. The linear range is then used to calculate the GEq/ml. Samples with higher concentrations than the linear range can be diluted as needed.

### Example 28: Tandem copies of the Anellovirus genome

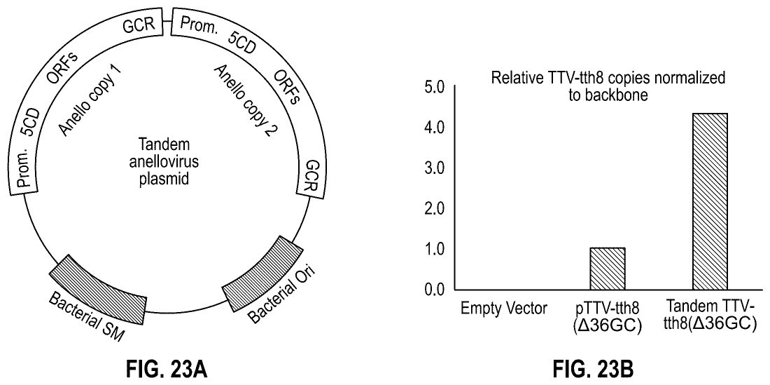

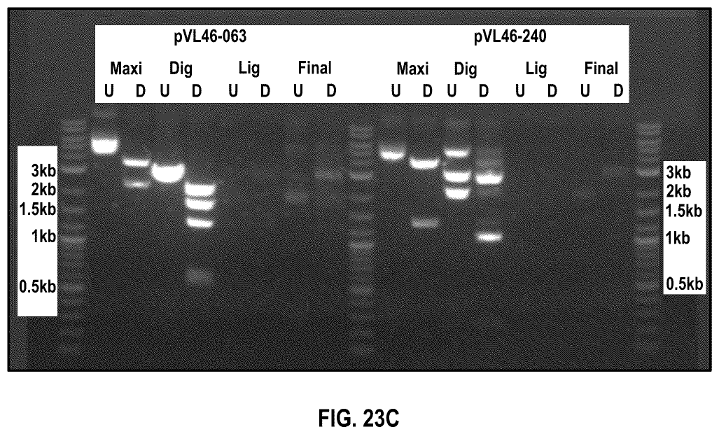

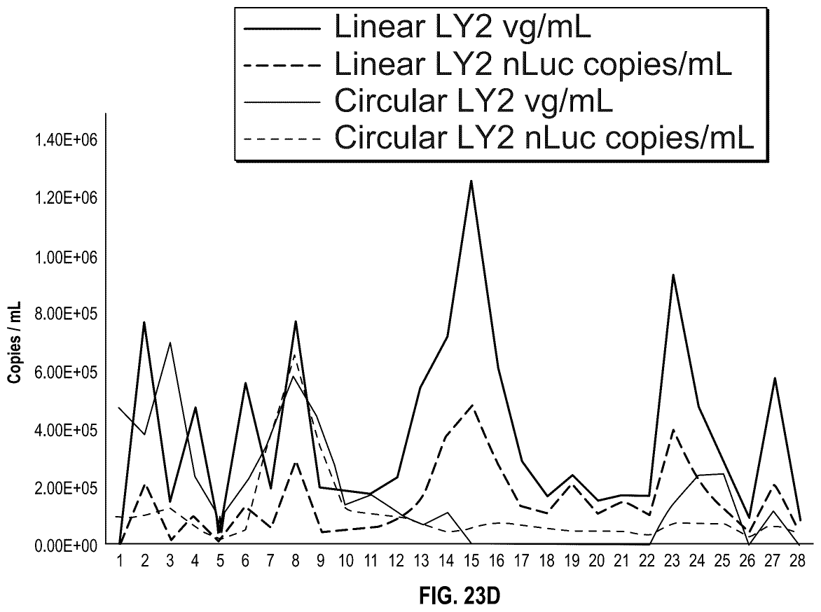

This example describes plasmid-based expression vectors harboring two copies of a single anelloviral genome, arranged in tandem such that the GC-rich region of the upstream genome is near the 5′ region of the downstream genome (FIG. 23A).

Anelloviruses replicate via rolling circle, in which a replicase (Rep) protein binds to the genome at an origin of replication and initiates DNA synthesis around the circle. For anellovirus genomes contained in plasmid backbones, this requires either replication of the full plasmid length, which is longer than the native viral genome, or recombination of the plasmid resulting in a smaller circle comprising the genome with minimal backbone. Therefore, viral replication off of a plasmid can be inefficient. To improve viral genome replication efficiency, plasmids are engineered with tandem copies of TTV-tth8 and TTMV-LY2. These plasmids present every possible circular permutation of the anelloviral genome: regardless of where the Rep protein binds, it will be able to drive replication of the viral genome from the upstream origin of replication to the downstream origin. A similar strategy has been used to produce porcine Anelloviruses (Huang et al., 2012, Journal of Virology 86 (11) 6042-6054).

Tandem anellovector can be assembled, for example, by sequentially cloning copies of the genome into a plasmid backbone, leaving 12 bp of non-viral DNA between the two sequences. Alternatively, tandem anellovector can be assembled via Golden-gate assembly, simultaneously incorporating two copies of the genome into a backbone and leaving no extra nucleotides between the genomes.

Plasmid harboring tandem copies of an anellovector genetic element sequence is transfected into HEK239T cells. Cells are incubated for five days, then lysed using 0.1% Triton X-100 and treated with nucleases to digest DNA not protected by viral capsids. qPCR is then performed using Taqman probes for the TTV-tth8 genome sequence and the plasmid backbone. TTV-tth8 genome copies are normalized to backbone copies.

### Example 29: In vitro circularized Anellovirus genomes

This example describes constructs comprising circular, double stranded Anelloviral genome DNA with minimal non-viral DNA. These circular viral genomes more closely match the double-stranded DNA intermediates found during wild-type Anellovirus replication. When introduced into a cell, such circular, double stranded Anelloviral genome DNA with minimal non-viral DNA can undergo rolling circle replication to produce, for example, a genetic element as described herein.

In one example, plasmids harboring an Anellovirus genome sequence are digested with restriction endonucleases recognizing sites flanking the genomic DNA. The resulting linearized genomes are then ligated to form circular DNA. These ligation reactions are done with varying DNA concentrations to optimize the intramolecular ligations. The ligated circles are either directly transfected into mammalian cells, or further processed to remove non-circular genome DNA by digesting with restriction endonucleases to cleave the plasmid backbone and exonucleases to degrade linear DNA. To demonstrate the improvements in Anellovirus production, circularized Anellovirus genome constructs are transfected into HEK293T cells. After 7 days of incubation, cells are lysed, and qPCR is performed to compare the levels of anellovirus genome between circularized and plasmid-based anelloviral genomes. Increased levels of Anelloviral genomes show that circularization of the viral DNA is a useful strategy for increasing Anellovirus production.

Digested plasmid can be purified on 1% agarose gels prior to electroelution or Qiagen column purification and ligation with T4 DNA Ligase. Circularized DNA is concentrated on a 100 kDa UF/DF membrane before transfection. Circularization is confirmed by gel electrophoresis. T-225 flasks are seeded with HEK293T at 3×10⁴ cells/cm² one day prior to lipofection with Lipofectamine 2000. Nine micrograms of circularized Anellovirus DNA and 50 g of circularized Anellovirus-nLuc are co-transfected one day post flask seeding. As a comparison, an additional T-225 flask is co-transfected with 50 g of linearized Anellovirus and 50 g of linearized Anellovirus-nLuc.

Anellovector production proceeds for eight days prior to cell harvest in Triton X-100 harvest buffer. Generally, anellovectors can be enriched, e.g., by lysis of host cells, clarification of the lysate, filtration, and chromatography. In this example, harvested cells are nuclease treated prior to sodium chloride adjustment and 1.2 m/0.45 m normal flow filtration. Clarified harvest is concentrated and buffer exchanged into PBS on a 750 kDa MWCO mPES hollow fiber membrane. The TFF retentate is filtered with a 0.45 m filter before loading on a Sephacryl S-500 HR SEC column pre-equilibrated in PBS. Anellovectors are processed across the SEC column at 30 cm/hr. Individual fractions are collected and assayed by qPCR for viral genome copy number and transgene copy number. Viral genomes and transgene copies are observed beginning at the void volume, Fraction 7, of the SEC chromatogram. Agreement between copy number for Anellovirus genomes and Anellovirus-nLuc transgene for Anellovectors produced using circularized input DNA at Fraction 7-Fraction 10 indicates packaged Anellovectors containing nLuc transgene. SEC fractions are pooled and concentrated using a 100 kDa MWCO PVDF membrane and then 0.2 m filtered prior to in vivo administration.

### Example 30: Production of anellovectors containing chimeric ORF1 with hypervariable domains from different Torque Teno Virus strains

This example describes domain swapping of hypervariable regions of ORF1 to produce chimeric anellovectors containing the ORF1 arginine-rich region, jelly-roll domain, N22, and C-terminal domain of one TTV strain, and the hypervariable domain from an ORF1 protein of a different TTV strain.

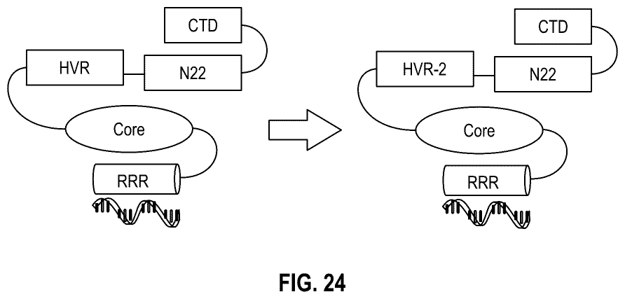

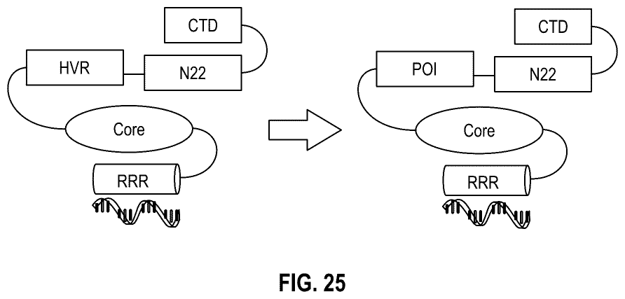

The full-length genome of a first Anellovirus is cloned into expression vectors for expression in mammalian cells. This genome is mutated to remove the hypervariable domain of the ORF1 coding sequence and replace it with the hypervariable domain of the ORF1 coding sequence from a second Anellovirus genome (FIG. 24). The plasmid containing the first Anellovirus genome with the swapped hypervariable domain is then linearized and circularized as described herein. HEK293T cells are transfected with the circularized genome and incubated for 5-7 days to allow anellovector production. After the incubation period anellovectors are purified from the supernatant and cell pellet of transfected cells by gradient ultracentrifugation.

To determine if the chimeric anellovectors are still infectious, the isolated viral particles are added to uninfected cells. The cells are incubated for 5-7 days to allow viral replication. After incubation the ability of the chimeric anellovectors to establish infection will be monitored by immunofluorescence, western blot, and qPCR. The structural integrity of the chimeric viruses is assessed by negative stain and cryo-electron microscopy. Chimeric anellovectors can further be tested for ability to infect cells in vivo. Establishment of the ability to produce functional chimeric anellovectors through hypervariable domain swapping could allow for engineering of viruses to alter tropism and potentially evade immune detection.

### Example 31: Design of an anellovector harboring a payload

This example describes the design of an exemplary anellovector genetic element harboring a trans gene. The genetic element is composed of the essential cis replication and packaging domains from an Anellovirus genome (e.g., as described herein) along with a non-Anellovirus payload, which may include, e.g., protein or non-coding RNA-expressing genes. The anellovector lacks essential trans protein elements for replication and packaging, and requires proteins provided by other sources (e.g., helpers, e.g., replicating viruses, expression plasmids, or genome integrations) for rolling circle replication and encapsidation.

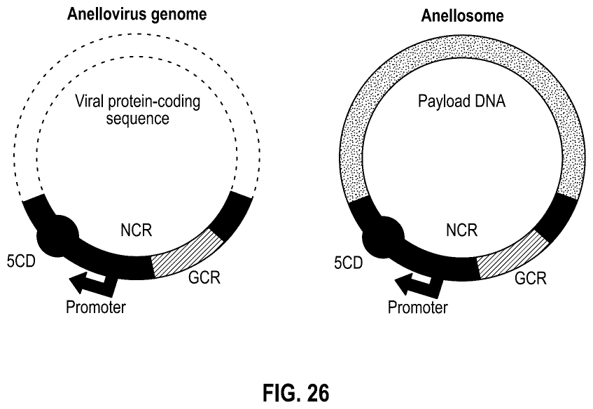

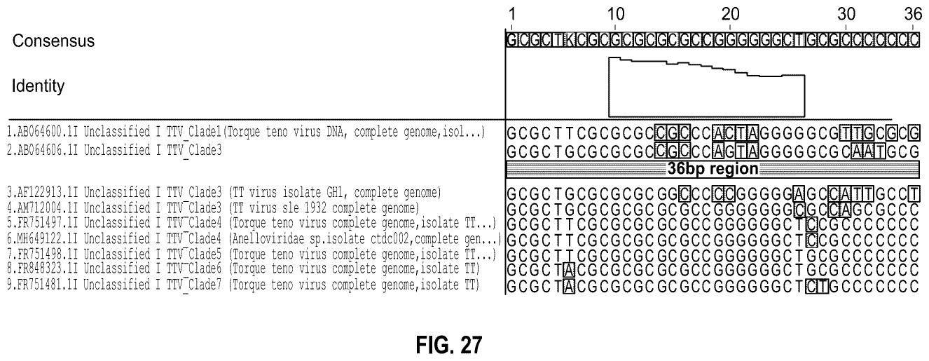

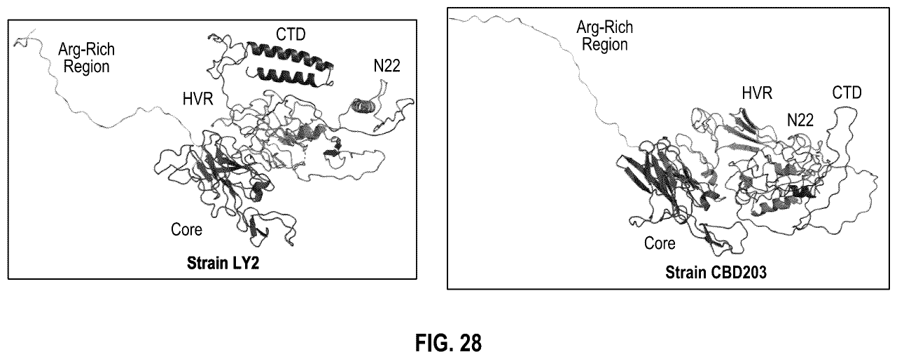

In one set of examples, the entire protein-coding DNA sequence is deleted, from the first start codon to the last stop codon (FIG. 26). The resulting DNA retains the viral non-coding region (NCR), including the viral promoter, the 5′ UTR conserved domain, the 3′ UTR (which encodes miRNAs in some anellovirus strains), and the GC-rich region. The anellovector NCR harbors essential cis domains, including the viral origin of replication and capsid binding domains. However, lacking the anellovirus protein-coding open reading frames, the anellovector is unable to express essential protein factors required for DNA replication and encapsidation, and therefore cannot amplify or package unless these elements are provided in trans.

Payload DNA, including but not limited to protein-encoding sequences, full trans genes (including non-anelloviral promoter sequences), and non-coding RNA genes are incorporated into the anellovector genetic element by insertion into the site of the deleted anelloviral open reading frames (FIG. 26). Expression from protein-coding sequences can be driven, for example, by either the native viral promoter or a synthetic promoter incorporated as a trans gene.

Replication-deficient or incompetent anellovector genetic elements (e.g., as described herein) may lack the protein-coding sequences for viral replication and/or capsid factors. Therefore, packaged anellovectors are produced by co-transfecting cells with the anellovector DNA described in this example and viral-protein-encoding DNA. The viral proteins are expressed off of replication-competent wild-type viral genomes, non-replicating plasmids harboring the viral proteins under control of the viral promoter, or plasmids harboring the viral proteins under control of a strong constitutive promoter.

### Example 32: In Vitro Circularized Genome as Input Material for Producing Anellovector Genetic Elements In Vitro

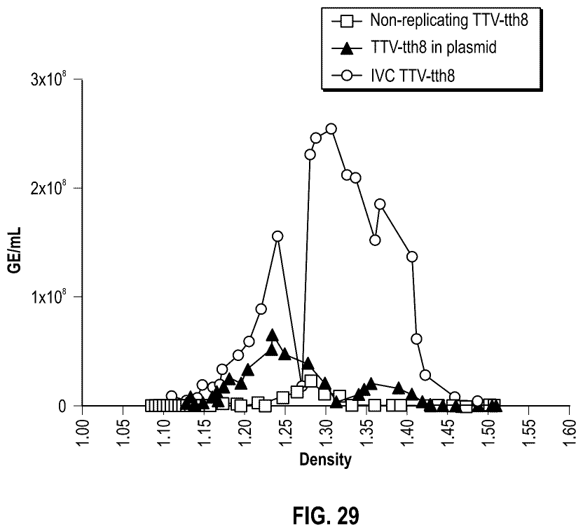

This example demonstrates that in vitro circularized (IVC) double stranded anellovirus DNA, as source material for an anellovector genetic element as described herein, is more robust than an anellovirus genome DNA in a plasmid to yield packaged anellovector genomes of the expected density. 1.2E+07 HEK293T cells (human embryonic kidney cell line) in T75 flasks were transfected with 11.25 ug of either, (i) in vitro circularized double stranded TTV-tth8 genome (IVC TTV-tth8), (ii) TTV-tth8 genome in a plasmid backbone, or (iii) plasmid containing just the ORF1 sequence of TTV-tth8 (non-replicating TTV-tth8). Cells were harvested 7 days post transfection, lysed with 0.1% Triton, and treated with 100 units per ml of Benzonase. The lysates were used for cesium chloride density analysis; density was measured and TTV-tth8 copy quantification was performed for each fraction of the cesium chloride linear gradient. As shown in FIG. 29, IVC TTV-tth8 yielded dramatically more viral genome copies at the expected density of 1.33 as compared to TTV-tth8 plasmid.

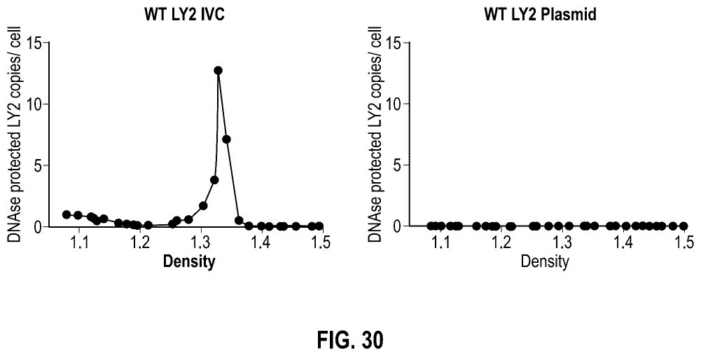

1E+07 Jurkat cells (human T lymphocyte cell line) were nucleofected with either in-vitro circularized LY2 genome (LY2 IVC) or LY2 genome in plasmid. Cells were harvested 4 days post transfection and lysed using a buffer containing 0.5% triton and 300 mM sodium chloride, followed by two rounds of instant freeze-thaw. The lysates were treated with 100 units/ml benzonase, followed by cesium chloride density analysis. Density measurement and LY2 genome quantification was performed on each fraction of the cesium chloride linear gradient. As shown in FIG. 30, transfection of in vitro circularized LY2 genome in Jurkat cells led to a sharp peak at the expected density, as compared to the transfection of plasmid containing the LY2 genome, which showed no detectable peak in FIG. 30.

In some embodiments, IVC anellovector genetic element constructs can be introduced into insect cells (e.g., Sf9 cells) and enclosed within proteinaceous exterior proteins expressed from a bacmid, e.g., as described herein.

### Example 33: Antibody Generation and Western Blot Analysis

This example describes generation of Ring 10 (also referred to herein as Lyl) antibodies and western blot analysis for determining expression, e.g., of Ring 10 ORF1 proteins. Ring 10 antibodies were generated by immunizing rabbits with a synthetic peptide representing one of three portions of the ORF1 protein (jelly roll residues 46-58: KIKRLNIVEWQPK, spike domain residues 485-502: SPSDTHEPDEEDQNRWYP, C-terminal domain residues 635-672: SEEEEESNLFERLLRQRTKQLQLKRRIIQTLKDLQKLE) conjugated to a carrier protein by an engineered N-terminal cysteine. Rabbits were immunized twice, and polyclonal antibodies were purified from bleeds using protein A purification (Custom Antibody Production, Life Technologies corporation). Western blot analysis was performed using NUPAGE 4-12% gels (ThermoFisher) transferred to nitrocellulose membranes using the Transblot Turbo system (BioRad). Membranes were blocked with blocking buffer (Licor), probed ˜16 hours with primary antibody, and detected using anti-rabbit IRDye infrared secondary antibodies and imaging system (Licor).

### Example 34: Construct Design, Cell Culture and Protein Expression/Purification

This example describes design of Ring 10 constructs, cell culture, and expression/purification of Ring 10 proteins. The Ring 10 ORF2 and ORF1 sequences were codon optimized for insect cells and with different ORF1 construct length variations (full-length ORF1, delARM with a deletion of residue 2-45, and delARM/delCTD with deletions of residues 2-45 and 552-672). The ORF2 and ORF1 constructs were cloned into the pFastBac Dual plasmid which was used to generate baculoviruses. To express the ORF1 proteins, Sf9 cells (GibcoT^{M} 11496015) were infected by baculovirus with multiplicity of infection=1 and the cells were cultured for three days at 27° C. and harvested by centrifugation. The cells were lysed by treatment with 0.01% Triton X-100 (Sigma-Aldrich 11332481001), subjected to micro-fluidization, and treated with protease inhibitors (Thermo Scientific Halt Protease Inhibitor Cocktail, PI78438) and DNase (Benzonase®; Sigma). Cell lysate was subsequently purified using HiTrap Heparin affinity chromatography (Cytiva) followed by size-exclusion chromatography (HiPrep 16/60 Sephacryl S-500 HR; Cytiva).

### Example 35: Negative-Stained EM Data Collection and Analysis

This example describes visualization of anellovirus particles using negative stain electron microscopy. Jeol 1200EX transmission electron microscope was used for screening different Ring 10 constructs. 10 μl of sample was blotted on 400 mesh carbon support film (cf400-cu, EMS) for 30 seconds. After washing by ddH₂0 for 30 seconds, the grid was stained by 0.75% of uranyl formate (UF) for 10 seconds before loading on the scope. Ring 10 delARM was further imaged at NanoImaging Service. 3 μl of 0.12 mg/ml was blotted to a continuous carbon grid and stained by 1% UF. Negative-stained electron microscopy was performed using a Thermo Fisher Scientific (Hillsboro, Oregon) Glacios Cryo Transmission Electron Microscope (cryo-TEM) operated at 200 kV and equipped with a TFS CETA-D 4×4 CMOS camera and a Falcon 4 direct electron detector.

### Example 36: Cryo-EM Data Collection and Data Analysis and Molecular Refinement

This example describes performing cryogenic electron microscopy (Cryo-EM) and data analysis, e.g., for determination of anellovirus particle structure. For the grid preparation, 3 μl of 0.3 mg/ml VLP sample was applied to a 1.2×1.3 graphene oxide grid. A total of 11,083 micrographs were collected from Glacios cryo-TEM (Thermo Fisher Scientific) operated at 200 kV with a Falcon 4 direct electron detector, at a nominal defocus range of −1.0-2.5 m and accumulated dose of 19.59 e/Å for a total of 15 frames in 3 minutes. The pixel size was 0.923 Å, with the magnification 150000×. Automated data-collection was carried out by Leginon software.

All micrographs were motion-corrected by Relion-4.0 implemented MotionCor2, and the contrast transfer function (CTF) parameters were estimated by Gctf. With manually picked particles from 20 micrographs to train the network, SPHIRE-crYOLO automatically picked 58,391 particles along with the PhosaurusNet network. All particles were extracted by Relion-4.0 and rescaled to 2-fold (pixel size 1.846 Å), followed by subsequence 2D classifications with 350 Å mask diameter to remove any junk particles. Two iterations of 2D classifications resulted in 11,185 particles, which were merged and reextracted to generate a de novo 3D initial model by Relion-4.0 with II symmetry. Notably, several similar initial models without symmetry imposed were obtained by Relion-4.0 and cisTEM. To obtain a better classification result, all particles were first subjected to a Refine3D with initial angular sampling 3.7° and local angular search 0.9° per step. The alignment parameters of each particle were transferred to a 3D classification with angular sampling interval 0.9° and local angular search 5° per step. 3D classification of the entire particle set attributed most of the particles into a single class. After CtfRefine and Bayesian polishing, the post-processing results in 3.98 Å resolution under the gold standard (with FSC=0.143).

The initial anellovirus TTMV-LY2 capsid monomer structure was predicted by TrRosetta, and Ring 10 structure was further predicted by RosettaCM. Structural refinement was performed by Rosetta and Phenix, and fine-adjusted by COOT. JA 20 and SAFIA structures are predicted by Alphafold.

### Example 37: Circular Dichroism Spectroscopy

This example describes performing circular dichroism spectroscopy to determine secondary structure of, e.g., an anellovirus ORF1 protein. To determine the secondary structure of the Ring 10 C-terminal domain, the peptides used to generate C-terminal antibodies (residues 635-672) were analyzed at 25.8 μM in PBS on a Jasco J-815 circular dichroism spectropolarimeter using 2 mm path length cell at ambient temperature. Each data set is the average of three consecutive scans. The secondary structure was determined by using CDPro software package, compared with a reference set containing 56 proteins (IBasis=10). The final secondary structure fractions were averaged over the results from three programs (SELCON3, CDSSTR, CONTINLL) in CDPro.

### Example 38: Anellovirus Particle Structure

This example describes determination of the particle structure of an exemplary Anellovirus.

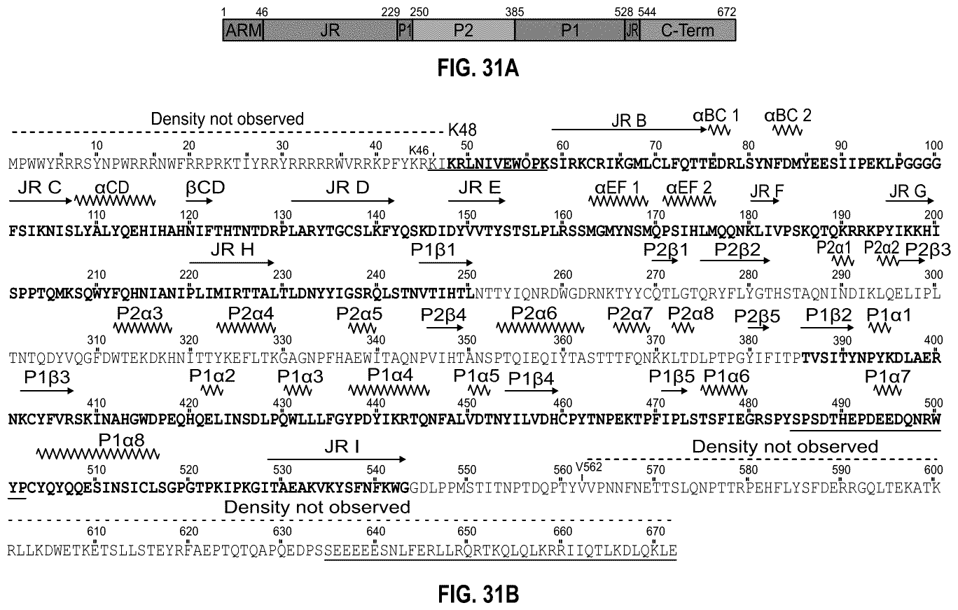

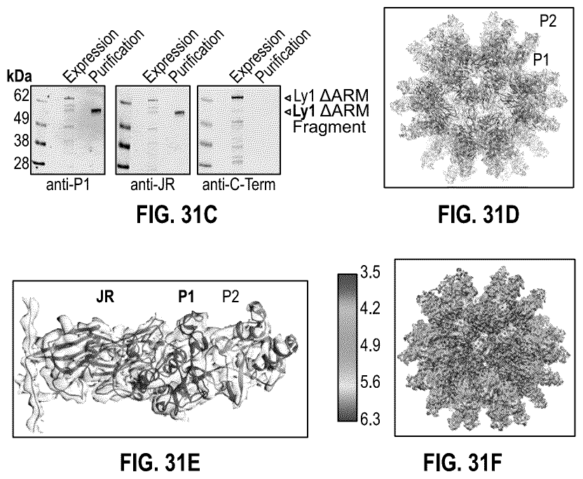

Initial efforts to study the structure of a virus-like particle (VLP) derived from a TTMV isolate described herein as Ring 10, were done with the full-length ORF1 (residues 1-672) expressed in insect cells (Sf9). Full-length ORF1 was visualized by electron microscopy (as described in Example 35) and was observed to assemble into particles −32 nm in diameter (FIG. 34A), similar to previously reported estimates of anellovirus size. However, full-length ORF1 particles lacked the homogeneous symmetry expected of viral particles (FIG. 34A). Genome packaging by ARM-containing viruses is believed to occur when the positive arginine residues bind a negatively charged genome, overcoming electrostatic repulsion of the ARM to permit ORF1 assembly. Therefore, Ring 10 delARM, an ORF1 construct wherein residues 2-45 are deleted, was designed as described in Example 34 (FIGS. 31A, 31B, and 34B) to facilitate more efficient ORF1 assembly in the absence of viral DNA. Ring 10 delARM expression was confirmed using a rabbit polyclonal antibody raised against a spike domain peptide (residues 485-502;

FIG. 31B) as described in Example 33. A band consistent with Ring 10 delARM was observed by western blot analysis (expected mass of 73.3 kDa; FIG. 31C) performed as described in Example 33. Ring 10 delARM yielded an ORF1 band above the 62-kDa marker (FIG. 31C). The difference in apparent molecular weight of Ring 10 delARM ORF1 after initial expression and after purification suggested proteolysis. To identify the site of proteolysis, polyclonal antibodies to peptides from the extreme N- and C-termini of Ring 10 delARM (residues 46-58 and residues 635-672, respectively; FIG. 31B) were generated as described in Example 33, and the presence of the N-terminal peptide of Ring 10 delARM on the purified fragment and the absence of the C-terminal peptide were confirmed (FIG. 31C).

Electron microscopy (EM) analysis of the Ring 10 delARM fragment showed the VLPs formed were more homogeneous and symmetric in morphology relative to full-length ORF1 (FIGS. 34A and 34B). The formation of homogeneous VLPs following genetic removal of the N-terminus and proteolysis of the C-terminus has been observed in another JR-containing virus, hepatitis E (HEV). To determine if the C-terminus of Ring 10 ORF1 is required for particle formation, VLPs from construct Ring 10 ORF1 delARM delCTD (wherein residues 2-45 and 552-672 are deleted) were generated as described in Example 34. Ring 10 ORF1 delARM delCTD produced VLPs of similar symmetry to Ring 10 ORF1 delARM (FIGS. 34B and 34C). These results suggest that proteolysis of the ORF1 C-terminus may be a natural part of anellovirus formation. In light of recent evidence that the C-terminal region of ORF1 is the immunodominant region of anelloviruses, its excision from the mature particle would be consistent with the immune evasion properties of anelloviruses.

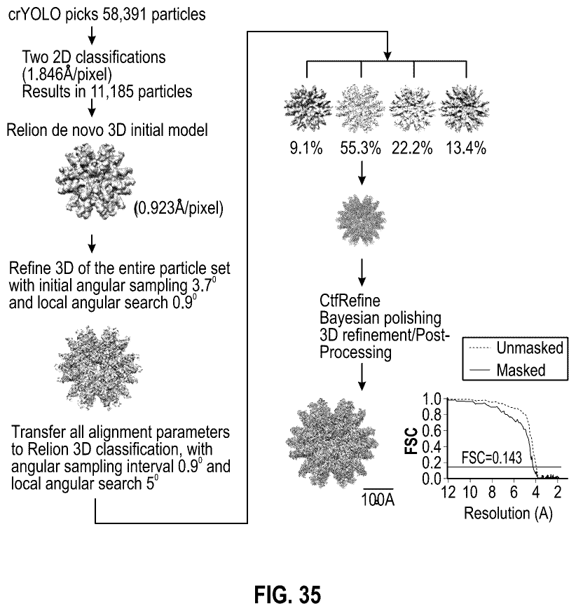

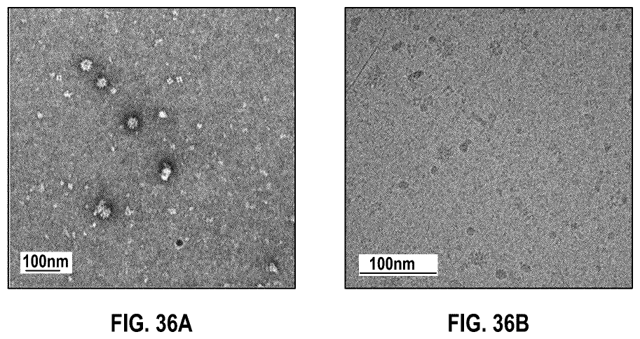

The structure of the Ring 10 delARM particle was determined using cryo-EM to 3.98 Å resolution as described in Example 36 (FIGS. 31D-31F, 35, 36A, and 36B; Table Si). The anellovirus particle was shown to be formed by sixty ORF1 fragments organized in an icosahedral T=1 symmetry (FIG. 31D). Electron density for residues 48-562 was observed (FIG. 31B). The resulting mass of the observed Ring 10 fragment was calculated to be ˜59.8 kDa. The N-terminal region (residues 46-228) forms part of the canonical 8-β-strand JR domain (P strands named B to H by convention). Unexpectedly, the eighth and final β-strand in the JR (strand I, residues 531-542) is located just prior to the C-terminal domain (FIG. 31B). The resulting fold of the ORF1 protomer has residues at the N- and C-termini generating the JR domain at the particle core while the intrastrand residues form the exterior of the particle surface. Intrastrand insertions forming the viral particle exterior can be found in other JR-containing viruses such as adeno-associated virus (AAV) and canine parvovirus (CPV). However, while the intrastrand insertions for AAV2 (228 residues) and CPV (227 residues) are between G and H β-strands, the 298-residue intrastrand insertion in Ring 10 is significantly larger and lies between β-strands H and I. The intrastrand region (residues 229-530) extends from the JR domain to form a structure herein referred to as a spike domain. The spike domain is formed by two globular domains: the spike P1 domain (residues 229-250 and 386-530) and the spike P2 domain (residues 251-385; FIG. 31B, 31E).

TABLE S1
Cryo-EM data collection, refinement and validation
statistics of TTMV-Ring 10 delARM.

| | Ring 10 delARM |
|---|---|
| Data collection and processing | |
| Magnification | 150000x |
| Voltage (kV) | 200 |
| Electron exposure (e-/Å²) | 1.31 |
| Defocus range (um) | −0.5~−2.5 |
| Pixel size (Å) | 0.923 |
| Symmetry imposed | I1 |
| Initial particle images (no.) | 58391 |
| Final particle images (no.) | 6271 |
| Map resolution (Å) | 3.98/0.143 |
| FSC threshold | |
| Map resolution range (Å) | 3.9-6 |
| Refinement | |
| Initial model used (PDB code) | |
| Model resolution (Å) | 3.98/0.143 |
| FSC threshold | |
| Model resolution range (Å) | 3.9-6 |
| Map sharpening B factor (Å²) | −152.851 |
| Model composition | 253440/30900/0 |
| Non-hydrogen atoms | |
| Protein residues | |
| Ligands | |
| B factors (Å²) | 30.00/243.72/133.17 |
| Protein | |
| Ligand | |
| R.m.s. deviations | 0.007/1.282 |
| Bond lengths (Å) | |
| Bond angles (°) | |
| Validation | 2.36/25.43/0 |
| MolProbity score | |
| Clashscore | |
| Poor rotamers (%) | |
| Ramachandran plot | 92.6/6.62/0.78 |
| Favored (%) | |
| Allowed (%) | |
| Disallowed (%) | |
| CC (mask) | 0.81 |
| MolProbility | 2.36 |
| Clash Score | 25.43 |

### Example 39: Anellovirus Structural Jelly Roll Domains

This example describes the structure of anellovirus structural jelly roll (JR) domains. Sixty Ring 10 JR domains form the core of the virus particle (FIG. 32A-32D). The β-strands form β-sheets which are characterized by a C-H-E-F pattern on the core's exterior and B-I-D-G pattern on the core's interior. The N-terminus of strand B is oriented to place the ARM on the interior of the core, where it is positioned to bind the viral genome. The observed C-terminal residues (545-562) extend from the C-terminus of β-strand I on the interior of the particle and thread through JR domains on the 2-fold axis to contact the neighboring JR domain (FIG. 32B).

In several JR-containing viruses, positively charged residues (arginine and lysine) oriented internally on strands B, I, D, and G are expected to bind the negatively charged viral genome (FIG. 32C). In Ring 10, basic residues Arg61, Lys62, Arg64, Lys66 (β-strand B), Lys140 (β-strand D), Arg197 (β-strand G), Lys533, Lys535, and Lys541 (β-strand I) are all oriented toward the particle interior and are likely responsible, together with the ARM motif, for binding the negatively charged viral genome.

Notably, density suggesting bound nucleic acid was not observed, possibly because the ARM deletion prevented nucleic acid binding or because any host cell nucleic acid encapsidated by the VLP would be too heterogeneous for detection. Alignment of anellovirus ORF1 sequences revealed that several of these putative DNA-binding residues are conserved across species, which supports their role in DNA-binding (FIG. 32D).

### Example 40: Anellovirus Spike Domains

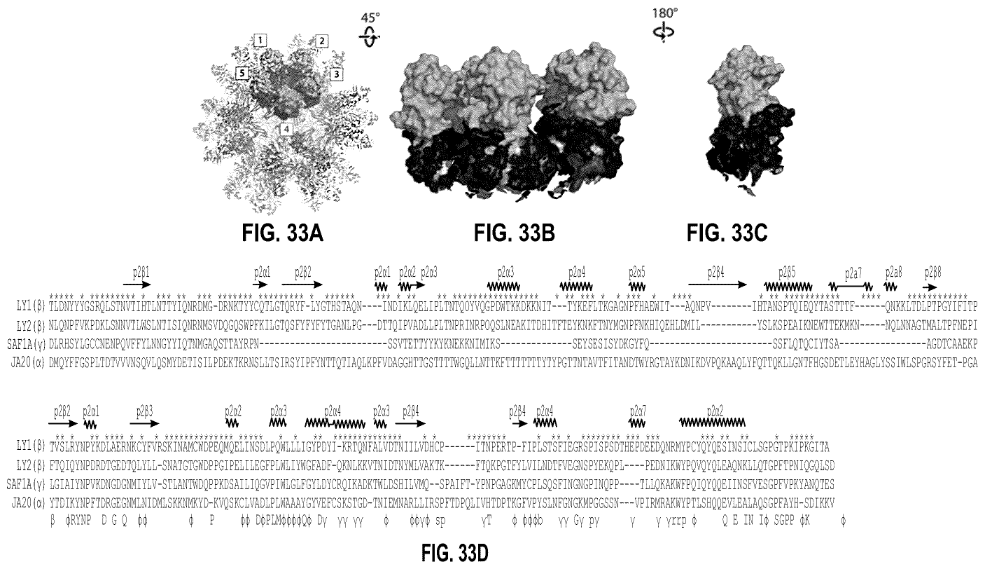

This example describes the structure of exemplary anellovirus spike domains, which include the P1 and P2 domains (e.g., as described herein). Residues 229 to 530 form the spike domain that extends −6 nm from the JR core (FIG. 33A-33D). A P strand (residues 245-250) extending from JR 3-strand H is the first component of the spike P1 domain and is N-terminal to the spike P2 domain (residues 251-385). The previously described hypervariable region (HVR) of ORF1 comprises the majority of the spike P2 domain. The remaining residues of the spike domain (residues 386-530) form five additional 1-strands and eight helices, which, together with the residues 245-250 strand, fold into the spike P1 domain. The local resolution of P1 is only slightly lower than the JR domain (˜4˜4.5 Å), while the resolution of P2 is within 5-6 Å. This is likely a consequence of both being further from the radius of gyration and some flexibility of the HVR residues.

Neighboring spike domains pack together around the five-fold symmetry axis to form a ringed structure of 5 spike domains henceforth called the crown (FIG. 33A, 33B). A receptor for anelloviruses has not been identified to date. Given the diverse tropism of different anellovirus strains, it is possible that residues of the spike P2 HVR, which are the most surface-exposed, are involved in viral attachment and infection. However, the hypervariable sequence of the spike P2 domain may serve to aid in immune evasion rather than harboring a receptor-binding motif. If this is the case, a receptor-binding motif on the better conserved spike P1 domain, or even on the surface of the JR core, may exist.

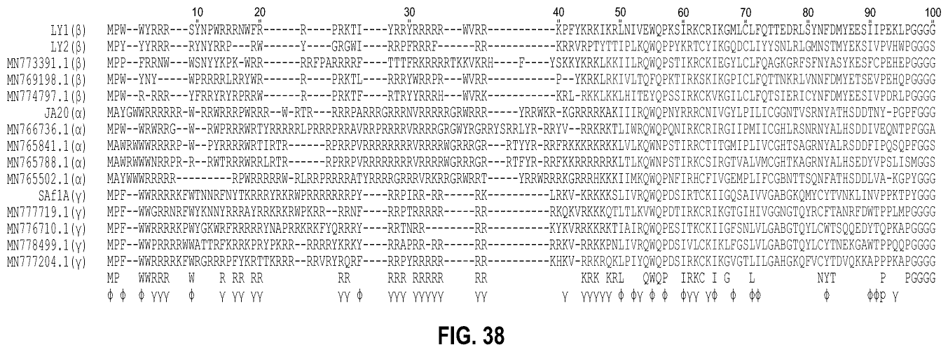

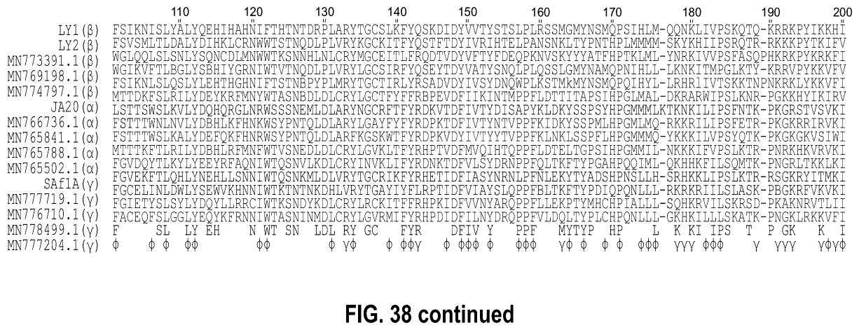

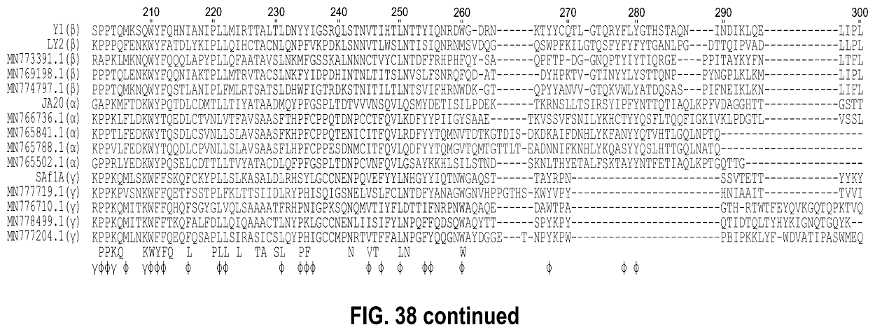

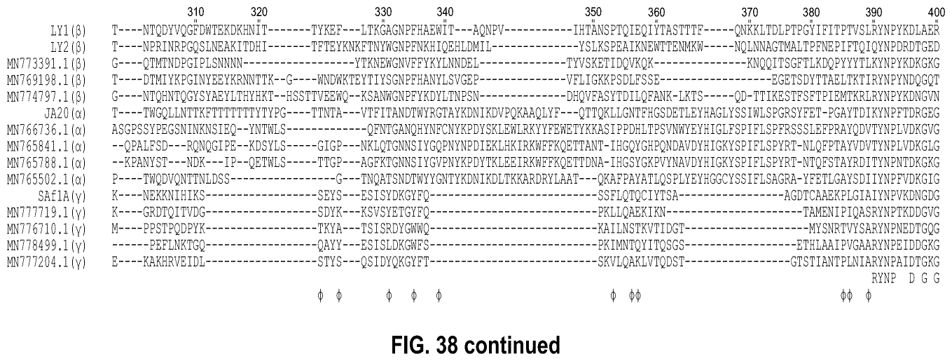

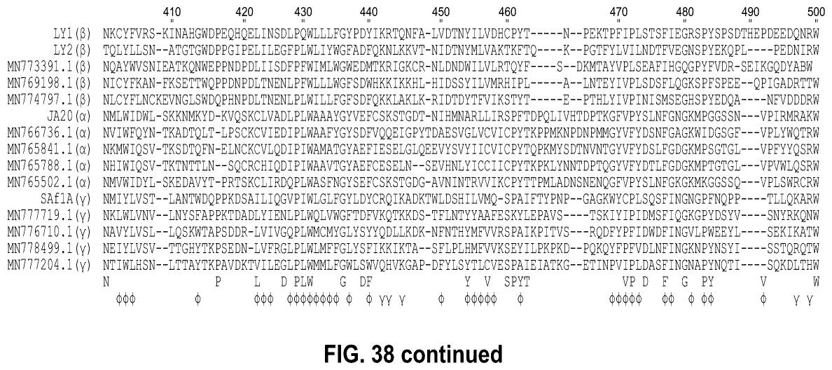

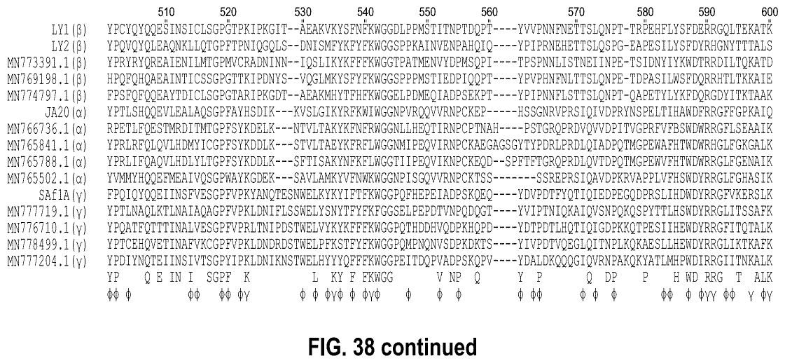

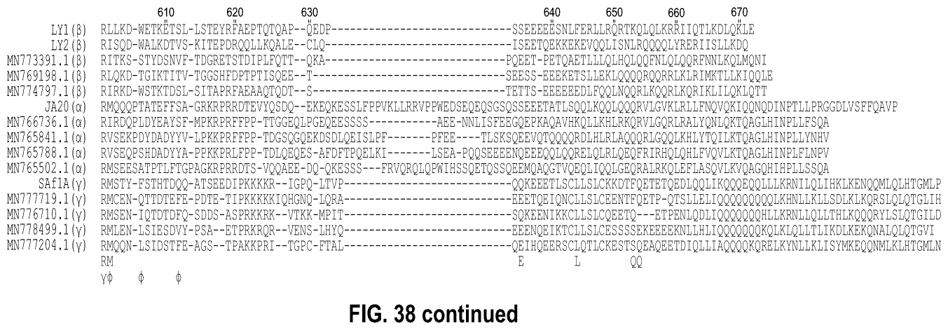

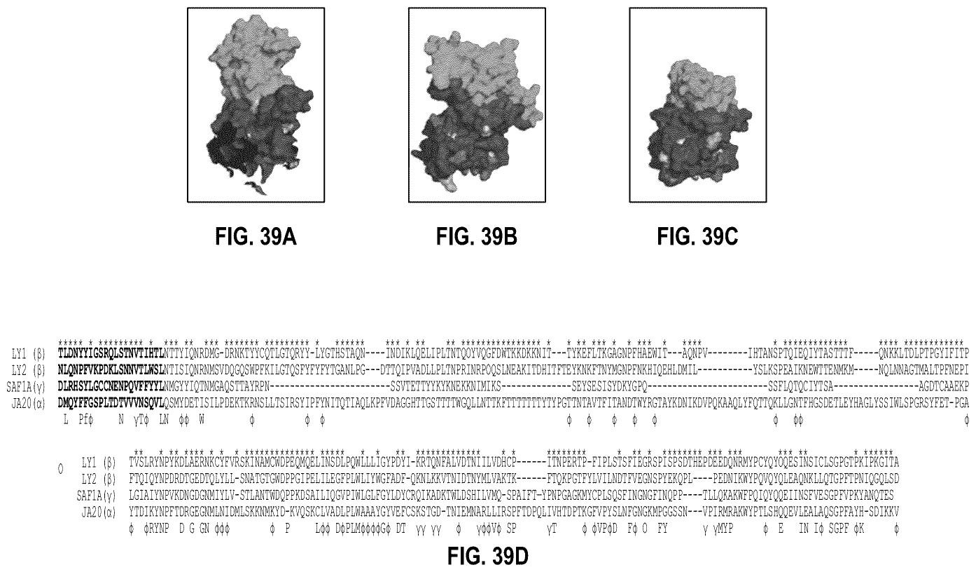

Sequence alignment of the Ring 10 spike domain with other anelloviruses did not readily identify conserved spike surface residues (FIG. 33B-33D, FIG. 38; sequences reproduced below). In fact, the Ring 10 residues that are more conserved between other anelloviruses (e.g., Asp396, Pro429, Trp431, Gly437, Pro461, Phe477, Pro483, Trp500, Tyr501, Pro502, Gly518 and Pro519) are internal spike P1 residues supporting its globular fold. A few basic residues are semi-conserved on the spike P1 surface (e.g., Arg390, Arg481, Arg499, and Arg523) which could contribute to the particle's ability to bind heparin resin. A few semi-conserved hydrophobic or aromatic residues on spike P1 (e.g., Leu231, I1e236, Val245, Tyr404, Gly414, I1e424, Leu428, Leu432, Val450, I1e478, and Tyr484) are at least partially surface-exposed and could represent a conserved receptor binding surface (FIG. 33B-33D). If this is the case, then it is attractive to posit that anelloviruses have evolved their novel elongated H-I intrastrand spike domain to sterically hinder antibody binding to their cellular receptor-binding site present on the P1 surface using domain P2 which is able to tolerate highly diverse amino acid substitutions. This would allow diverse anelloviruses to repeatedly infect human hosts with minimal recognition and neutralization by the immune system.

Sequences included in sequence alignments (FIG. 38)

>Ly1 (Ring10)_orf1
MPWWYRRRSYNPWRRRNWFRRPRKTIYRRYRRRRRWVRRKPFYKRKIKRLNIVEWQPKSIRKCRIKGMLCLFQT
TEDRLSYNFDMYEESIIPEKLPGGGGFSIKNISLYALYQEHIHAHNIFTHTNTDRPLARYTGCSLKFYQSKDID
YVVTYSTSLPLRSSMGMYNSMQPSIHLMQQNKLIVPSKQTQKRRKPYIKKHISPPTQMKSQWYFQHNIANIPLL
MIRTTALILDNYYIGSRQLSTNVTIHTLNTTYIQNRDWGDRNKTYYCQTLGTQRYFLYGTHSTAQNINDIKLQE
LIPLINTQDYVQGFDWTEKDKHNITTYKEFLTKGAGNPFHAEWITAQNPVIHTANSPTQIEQIYTASTTTFQNK
KLTDLPTPGYIFITPTVSLRYNPYKDLAERNKCYFVRSKINAHGWDPEQHQELINSDLPQWLLLFGYPDYIKRT
QNFALVDTNYILVDHCPYTNPEKTPFIPLSTSFIEGRSPYSPSDTHEPDEEDQNRWYPCYQYQQESINSICLSG
PGTPKIPKGITAEAKVKYSFNFKWGGDLPPMSTITNPTDQPTYVVPNNFNETTSLQNPTTRPEHFLYSFDERRG
QLTEKATKRLLKDWETKETSLLSTEYRFAEPTQTQAPQEDPSSEEEEESNLFERLLRQRTKQLQLKRRIIQTLK
DLQKLE
>Ly2 (Ring2)_orf1
MPYYYRRRRYNYRRPRWYGRGWIRRPFRRRFRRKRRVRPTYTTIPLKQWQPPYKRTCYIKGQDCLIYYSNLRLG
MNSTMYEKSIVPVHWPGGGSFSVSMLILDALYDIHKLCRNWWTSTNQDLPLVRYKGCKITFYQSTFTDYIVRIH
TELPANSNKLTYPNTHPLMMMMSKYKHIIPSRQTRRKKKPYTKIFVKPPPQFENKWYFATDLYKIPLLQIHCTA
CNLQNPFVKPDKLSNNVTLWSLNTISIQNRNMSVDQGQSWPFKILGTQSFYFYFYTGANLPGDITQIPVADLLP
LINPRINRPGQSLNEAKITDHITFTEYKNKFTNYWGNPFNKHIQEHLDMILYSLKSPEAIKNEWTTENMKWNQL
NNAGTMALTPFNEPIFTQIQYNPDRDTGEDTQLYLLSNATGTGWDPPGIPELILEGFPLWLIYWGFADFQKNLK
KVTNIDTNYMLVAKTKFTQKPGTFYLVILNDTFVEGNSPYEKQPLPEDNIKWYPQVQYQLEAQNKLLQTGPFTP
NIQGQLSDNISMFYKFYFKWGGSPPKAINVENPAHQIQYPIPRNEHETTSLQSPGEAPESILYSFDYRHGNYTT
TALSRISQDWALKDTVSKITEPDRQQLLKQALECLQISEETQEKKEKEVQQLISNLRQQQQLYRERIISLLKDQ
>MN773391.1|Beta|245_2239_2_fow
MPPFRRNWWSNYYKPKWRRRRFPARRRRFTTTFRKRRRRTKKVKRHFYSKKYKRKLKKIILRQWQPSTIKRCKI
EGYLCLFQAGKGRFSFNYASYKESFCPEHEPGGGGWGLQQLSLSNLYSQNCDLMNWWTKSNNHLNLCRYMGCEI
TLFRQDTVDYVFTYFDEQPKNVSKYYYATFHPTKLMLYNRKIVVPSFASQPHKRKPYKRKFIRAPKLMKNQWYF
QQQLAPYPLLQFAATAVSLNKMFGSSKALNNNCTVYCLNTDFFRHPHFQYSAQPFTPDGGNQPTYIYTIQRGEP
PITAYKYFNLTFLGQTMINDPGIPLSNNNNYTKNEWGNVFFYKYLNNDELTYVSKETIDQVKQKKNQQITSGFT
LKDQPYYYTLKYNPYKDKGKGNQAYWVSNIEATKQNWEPPNDPDLIISDFPFWIMLWGWEDMTKRIGKCRNLDN
DWILVLRTQYFSDKMTAYVPLSEAFIHGQGPYFVDRSEIKGQDYAHWYPRYRYQREAIENILMTGPMVCRADNI
NNIQSLIKYKFFFKWGGTPATMENVYDPMSQPITPSPNNLISTNEIINPETSIDNYIYKWDTRRDILTQKATDR
ITKSSTYDSNVFTDGRETSTDIPLFQTTQKAPQEETPETQAETLLLQLHQLQQFNLQLQQRFNNLKQLMQNI
>MN769198.1|Beta|265_2235_1_fow
MPWYNYWPRRRRLRRYWRRPRKTLRRRYWRRPRWVRRPYKRKLRKIVLTQFQPKTIRKSKIKGPICLFQTTNKR
LVNNFDMYETSEVPEHQPGGGGWGIKVFTLEGLYSEHIYGRNIWTVTNQDLPLVRYLGCSIRFYQSEYTDYVAT
YSNQLPLQSSLGMYNAMQPNIHLLLKNKITMPGLKTYKRRVPYKKVFVPPPTQLENKWYFQQNIAKTPLLMTRV
TACSLNKFYIDPDHINTNLTITSLNVSLFSNRQFQDATDYHPKTVGTINYYLYSTTQNPPYNGPLKLKMLIPLT
DTMIYKPGINYEEYKRNNTTKGWNDWKTEYTIYSGNPFHANYLSVGEPVFLIGKKPSDLFSSEEGETSDYTTAE
LIKTIRYNPYNDQGQTNICYFKANFKSETTWQPPDNPDLINENLPFWLLLWGFSDWHKKIKKHLHIDSSYILVM
RHIPLALNTEYIVPLSDSFLQGKSPFSPEEQPIGADRITWHPQFQHQAEAINTICSSGPGTTKIPDNYSVQGLM
KYSFYFKWGGSPPPMSTIEDPIQQPTYPVPHNFNLTTSLQNPETDPASILWSFDQRRHTLTKKAIERLQKDTGI
KTITVTGGSHFDPTPTISQEETSEESSEEEKETSLLEKLQQQQRQQRRLKLRIMKTLLKIQQLE
>MN774797.1|Beta|244_2262_1_fow
MPWRRRRYFRRYRYRPRRWRPRKTFRTRYYRRRHWVRKKRLRKKLKKLHITEYQPSSIRKCKVKGILCLFQTSI
ERICYNFDMYEESIVPDRLPGGGGFSIKNLSLQSLYLEHTHGHNIFTSTNEPYPLMRYTGCTIRLYRSADVDYI
VSYDNQWPLKSTMKMYNSMQPQIHYLLRHRIIVTSKKTNPNKRKLYKKVFIPPPTQMKNQWYFQSTLANIPLFM
LRTSATSLDHWFIGTRDKSTNITILILNTSVIFHRNWDKGTQPYYANVVGTQKVWLYATDQSASPIFNEIKLKN
LIFLGNTQHNTQGYSYAEYLTHYHKTHSSTTVEEWQKSANWGNPFYKDYLTNPSNDHQVFASYTDILQFANKLK
TSQDTTIKESTFSFTPIEMTKRLRYNPYKDNGVNNLCYFLNCKEVNGLSWDQPHNPDLINENLPLWILLFGFSD
FQKKLAKLKRIDTDYTFVIKSTYTEPTHLYIVPINISMSEGHSPYEDQANFVDDDKWFPSFQFQQEAYTDICLS
GPGTARIPKGDTAEAKMHYTFHFKWGGELPDMEQIADPSEKPTYPIPNNFLSTTSLQNPTQAPETYLYKFDQRG
DYITKTAAKRIRKDWSTKTDSLSITAPRFAEAAQTQDTSTETTSEEEEEEDLFQQLNQQRLKQQRLKQRIKLIL
QKLQTT
>AF122914.3|Alpha|417_2726_3_fow (JA20)
MAYGWWRRRRRRWRRWRRRPWRRRWRTRRRRPARRRGRRRNVRRRRRGRWRRRYRRWKRKGRRRRKAKIIIRQW
QPNYRRRCNIVGYLPILICGGNTVSRNYATHSDDTNYPGPFGGGMTTDKFSLRILYDEYKRFMNYWTASNEDLD
LCRYLGCTFYFFRHPEVDFIIKINTMPPFLDTTITAPSIHPGLMALDKRARWIPSLKNRPGKKHYIKIRVGAPK
MFTDKWYPQTDLCDMILLTIYATAADMQYPFGSPLTDTVVVNSQVLQSMYDETISILPDEKTKRNSLLTSIRSY
IPFYNTTQTIAQLKPFVDAGGHTTGSTTTTWGQLLNTTKFTTTTTTTYTYPGTTNTAVTFITANDTWYRGTAYK
DNIKDVPQKAAQLYFQTTQKLLGNTFHGSDETLEYHAGLYSSIWLSPGRSYFETPGAYTDIKYNPFTDRGEGNM
LWIDWLSKKNMKYDKVQSKCLVADLPLWAAAYGYVEFCSKSTGDINIHMNARLLIRSPFTDPQLIVHTDPTKGF
VPYSLNFGNGKMPGGSSNVPIRMRAKWYPTLSHQQEVLEALAQSGPFAYHSDIKKVSLGIKYRFKWIWGGNPVR
QQVVRNPCKEPHSSGNRVPRSIQIVDPRYNSPELTIHAWDFRRGFFGPKAIQRMQQQPTATEFFSAGRKRPRRD
TEVYQSDQEKEQKESSLFPPVKLLRRVPPWEDSEQEQSGSQSSEEETATLSQQLKQQLQQQRVLGVKLRLLFNQ
VQKIQQNQDINPTLLPRGGDLVSFFQAVP
>MN766736.1|Alpha|422_2692_2_fow
MPWWRWRRGWRWPRRRWRTYRRRRRLPRRRPRRAVRRPRRRRVRRRRGRGWYRGRRYSRRLYRRRYVRRKRKTL
IWRQWQPQNIRKCRIRGIIPMIICGHLRSNRNYALHSDDIVEQNTPFGGALSTTSWSLKVLYDQHQRGLNRWSS
SNEMLDLARYNGCRFTFYRDKVTDFIVTYDISAPYKLDKYSSPSYHPGMMMLKTKNKILIPSENTKPKGRSTVS
VKIKPPKLFLDKWYTQEDLCTVNLVTFAVSAASFTHPFCPPQTDNPCCTFQVLKDFYYPIIGYSAAETKVSSVF
SNILYKHCTYYQSFLTQQFIGKIVKLPDGTLVSSLASGPSSYPEGSNINKNSIEQYNTWLSQFNTGANQHYNFC
NYKPDYSKLEWLRKYYFEWETYKKASIPPDHLTPSVNWYEYHIGLFSPIFLSPFRSSSLEFPRAYQDVTYNPLV
DKGVGNVIWFQYNTKADTQLTLPSCKCVIEDIPLWAAFYGYSDFVQQEIGPYTDAESVGLVCVICPYTKPPMKN
PDNPMMGYVFYDSNFGAGKWIDGSGFVPLYWQTRWRPETLFQESTMRDITMTGPFSYKDELKNTVLTAKYKFNF
RWGGNLLHEQTIRNPCPTNAHPSTGRQPRDVQVVDPITVGPRFVFHSWDWRRGFLSEAAIKRIRDQPLDYEAYS
FMPKRPRFFPPTTGGEQLPGEQEESSSSAEENNLISFEEGQEPKAQAVHKQLLKHLRKQRVLGQRLRALYQNLQ
KTQAGLHINPLLFSQA
>MN765841.1|Alpha|389_2635_2_fow
MAWRWWWRRRRPWPYRRRRWRTIRTRRPRRPVRRRRRRRRVRRRRWGRRRGRRTYYRRRFRKKKRKRKKLVLKQ
WNPSTIRRCTITGMIPLIVCGHTSAGRNYALRSDDFIPQSQPFGGSFSTTTWNLNVLYDEHLKFHNKWSYPNTQ
LDLARYLGAYFYFYRDPKTDFIVTYNTVPPFKIDKYSSPMLHPGMLMQRKKRILIPSFETRPKGKRRIRVKIKP
PTLFEDKWYTQSDLCSVNLLSLAVSAASFKHPFCPPQTENICITFQVLRDFYYTQMNVTDTKGTDISDKDKAIF
DNHLYKFANYYQTVHTLGQLNPTQQPALFSDRQNQGIPEKDSYLSGIGPNKLQTGNNSIYGQPNYNPDIEKLHK
IRKWFFKQETTANTIHGQYGHPQNDAVDYHIGKYSPIFLSPYRTNLQFPTAYVDVTYNPLVDKGLGNKMWIQSV
TKSDTQFNELNCKCVLQDIPIWAMATGYAEFIESELGLQEEVYSVYIICVICPYTQPKMYSDTNVNTGYVFYDS
LFGDGKMPSGTGLVPFYYQSRWYPRLRFQLQVLHDMYICGPFSYKDDLKSTVLTAEYKFRFLWGGNMIPEQVIR
NPCKAEGAGSGYTYPDRLPRDLQIADPQTMGPEWAFHTWDWRHGLFGKGALKRVSEKPDYDADYYVLPKKPRFF
PPTDGSQGQEKDSDLQEISLPFPFEETLSKSQEEVQTQQQQRDLHLRLAQQQRLGQQLKHLYTQILKTQAGLHI
NPLLYNHV
>MN765502.1|Alpha|417_2624_3_fow
MAYWWWRRRRRRPWRRRRRWRLRRPRRRRATYRRRRGRRRVRKRRGRWRRTYRRWRRRKGRRRHKKKIIMKQWQ
PNFIRHCFIVGFMPLIFCGENTTSQNFATHSDDLVAKGPYGGGMTTTKFTLRILYDEHLRFMNFWTVSNEDLDL
CRYLGVKLTFYRHPTVDFMVQIHTQPPFLDTELTGPSIHPGMMILNKKKIFVPSLKTRPNRKHKVRVKIGPPRL
YEDKWYPQSELCDTTLLTVYATACDLQFPFGSPLTDNPCVNFQVLGSAYKKHLSILSINDSKNLTHYETALFSK
TAYYNTFETIAQLKPTGQTTGVTPTWQDVQNTTNLDSSGTNQATSNDTWYYGNTYKDNIKDLTKKARDRYLAAT
QKAFPAYATLQSPLYEYHGGCYSSIFLSAGRAYFETLGAYSDIIYNPFVDKGIGNMVWIDYLSKEDAVYTPRTS
KCLIRDQPLWASFNGYSEFCSKSTGDGAVNINTRVVIKCPYTTPMLADNSNENQGFVPYSLNFGKGKMKGGSSQ
VPLSWRCKWYVMMYHQQEFMEAIVQSGPWAYKGDEKSAVLAMKYVFNWKWGGNPISGQVVRNPCKTSSSSREPR
SIQAVDPKRVAPPLVFHSWDYRRGLFGHASIKRMSEESATPTLFTGPAGKRPRRDTSVQQAEEDQQKESSSFRV
QRQLQPWIHSSQETQSSQEEMQAQGTVQEQLIQQLGEQRALRKQLEFLASQVLKVQAGQHIHPLLSSQA
>MN765788.1|Alpha|401_2629_2_fow
MAWRWWWNRRPRRRWTRRRWRRLRTRRPRRPVRRRRRRRRVRRRRWGRRRGRRTFYRRRFKKRRRRRRKLTLKQ
WNPSTIRKCSIRGTVALVMCGHTKAGRNYALHSEDYVPSLISMGGSFSTTTWSLKALYDEFQKFHNRWSYPNTQ
LDLARFKGSKWTFYRDPKVDYIVTYYTVPPFKLNKLSSPFLHPGMMMQYKKKILVPSYQTKPKGKGKVSIWIKP
PVLFEDKWYTQQDLCPVNLLSLAVSAASFLHPFCPPESDNMCITFQVLQDFYYTQMGVTQMTGTTLTEADNNIF
KNHLYKQASYYQSLHTTGQLNATQKPANYSTNDKIPQETWLSTTGPAGFKTGNNSIYGVPNYKPDYTKLEEIRK
WFFKQETTDNAIHGSYGKPVYNAVDYHIGKYSPIFLSPYRTNTQFSTAYRDITYNPNTDKGKGNHIWIQSVTKT
NTTLNSQCRCHIQDIPIWAAVTGYAEFCESELNSEVHNLYICCIICPYTKPKLYNNTDPTQGYVFYDTLFGDGK
MPTGTGLVPVWLQSRWYPRLIFQAQVLHDLYLTGPFSYKDDLKSFTISAKYNFKFLWGGTIIPEQVIKNPCKEQ
DSPFTFTGRQPRDLQVTDPQTMGPEWVFHTWDWRRGLFGENAIKRVSEQPSHDADYYAPPKKPRLFPPTDLQEQ
ESAFDFTPQELKILSEAPQQSEEEENQEEEQQLQQRELQLRLQEQFRIRHQLQHLFVQVLKTQAGLHINPLFLN
PV
>MN779270.1|Gamma|312_2300_3_fow (SAfiA)
MPFWWRRRRKFWINNRFNYTKRRRYRKRWPRRRRRRRPYRRPIRRRRRKLRKVKRKKKSLIVRQWQPDSIRTCK
IIGQSAIVVGAEGKQMYCYTVNKLINVPPKTPYGGGFGVDQYTLKYLYEEYRFAQNIWTQSNVLKDLCRYINVK
LIFYRDNKTDFVLSYDRNPPFQLTKFTYPGAHPQQIMLQKHHKFILSQMTKPNGRLTKKLKIKPPKQMLSKWFF
SKQFCKYPLLSLKASALDLRHSYLGCCNENPQVFFYYLNHGYYIQTNWGAQSTTAYRPNSSVTETTYYKYKNEK
KNIHIKSSEYSESISYDKGYFQSSFLQTQCIYTSAAGDTCAAEKPLGIAIYNPVKDNGDGNMIYLVSTLANTWD
QPPKDSAILIQGVPIWLGLFGYLDYCRQIKADKTWLDSHILVMQSPAIFTYPNPGAGKWYCPLSQSFINGNGPF
NQPPTLLQKAKWFPQIQYQQEIINSFVESGPFVPKYANQTESNWELKYKYIFTFKWGGPQFHEPEIADPSKQEQ
YDVPDTFYQTIQIEDPEGQDPRSLIHDWDYRRGFVKERSLKRMSTYFSTHTDQQATSEEDIPKKKKRIGPQLTV
PQQKEEETLSCLLSLCKKDTFQETETQEDLQQLIKQQQEQQLLLKRNILQLIHKLKENQQMLQLHTGMLP
>MN778499.1|Gamma|300_2288_3_fow
MPFWWPRRRRWWATTRFKRRKPRYPKRRRRRRYKRKYRRAPRRRRRRRRKVRRKKPNLIVRQWQPDSIVLCKIK
LFGSLVLGAEGTQYLCYTNEKGAWTPPQQPGGGGFGIETYSLSYLYDQYLLRRCIWTKSNDYKDLCRYLRCKIT
FFRHPKIDFVVNYARQPPFLLEKPTYMNCHPIALLLSQHKRVILSKRSDPKAKNRVTLIIKPPKQMITKWFFTK
QFALFDLLQIQAAACTLNYPKLGCCNENLIISIFYLNPQFFQDSQWAQYTTSPYKPYQTIDTQLTYHYKIGNQT
GQYKPEFLNKTGQQAYYESISLDKGWFSPKIMNTQYITQSGSETHLAAIPVGAARYNPEIDDGKGNEIYLVSVT
TGHYTKPSEDNLVFRGLPLWLMFFGLYSFIKKIKTASFLPLHMFVVKSEYILPKPKDPQKQYFPFVDLNFINGK
NPYNSYISSTQRQTWYPTCEHQVETINAFVKCGPFVPKLDNDRDSTWELPFKSTFYFKWGGPQMPNQNVSDPKD
KTSYIVPDTVQEGLQITNPLKQKAESLLHEWDYRRGLIKTKAFKRMLENLSIESDVYPSAETPRKRQRVENSLH
YQEEENQEIKTCLLSLCESSSSEKEEEEKNLLHLIQQQQQQQKQLKLQLLTLIKDLKEKQNALQLQTGVI
>MN776710.1|Gamma|288_2279_3_fow
MPFWWRRRRKPWYGKWRFRRRRRYNAPRRKRKFYQRRRYRRINRRRRKYKVRRKKRKTIAIRQWQPESITKCKI
IGFSNLVLGAEGTQYLCWTSQQEDYTQPKAPGGGGFGCELINLDWLYSEWVKHNNIWTKININKDLVRYTGAYI
YFLRHPTIDFVIAYSLQPPFELTKFTYPDIQPQNLLLRKRKRIILSLASKPHGKRFVKVKIRPPKQMITKWFFQ
HQFSGYGLVQLSAAAATFRHPNIGPKSQNQMVTIYFLDITIFNRPNWAQAQEDAWTPAGTHRTWTFEYQVKGQT
QPKTVQMPPSTPQDPYKTKYATSISRDYGWWQKAILNSTKVTIDGTMYSNRTVYSARYNPNEDTGQGNAVYLVS
LLQSKWTAPSDDRLVIVGQPLWMCMYGLYSYYQDLLKDKNFNTHYMFVVRSPAIKPITVSRQDFYPFIDWDFIN
GVLPWEEYLSEKIKATWYPQATFQTTTINALVESGPFVPRLTNIPDSTWELVYKYKFFFKWGGPQTHDDHVQDP
KHQPDYDTPDTLHQTIQIGDPKKQTPESIIHEWDYRRGFITQTALKRMSENIQTDTDFQSDDSASPRKKRKVTK
KMPITSQKEENIKKCLLSLCQEETQETPENLQDLIQQQQQQQHLLKRNLLQLLTHLKQQQRYLSLQTGILD
>MN777204.1|Gamma|313_2331_1_fow
MPFWWRRRRKFWRGRRRPFYKRTTKRRRRRVRYRQRFRRPYRRRRRRRKHKVRRKRQKLPIYQWQPDSIRKCKI
KGVGTLILGAHGKQFVCYTDVQKKAPPPKAPGGGGFACEQFSLGGLYEQYKFRNNIWTASNINMDLCRYLGVRM
IFYRHPDIDFILNYDRQPPFVLDQLTYPLCHPQNLLLGKHKILLLSKATKPNGKLRKKVFIKPPKQMLNKWFFQ
EQFQSAPLLSIRASICSLQYPHIGCCMPNRTVTFFALNPGFYQQGNWAYDGGETNPYKPWPHIPKKLYFWDVAT
IPASWMEQEKAKHRVEIDLSTYSQSIDYQKGYFTSKVLQAKLVTQDSTGISTIANTPLNIARYNPAIDTGKGNT
IWLHSNLTTAYTKPAVDKTVILEGLPLWMMLFGWLSWVQHVKGAPDFYLSYTLCVESPAIEIATKGETINPVIP
LDASFINGNAPYNQTISQKDLTHWYPDIYNQTEIINSIVTSGPYIPKLDNIKNSTWELHYYYQFFFKWGGPEIT
DQPVADPSKQPVYDALDKQQQGIQVRNPAKQKYATLMHPWDIRRGIITNKALKRMQQNLSIDSTFEAGSTPAKK
PRITGPCFTALQEIHQEERSCLQTLCKESTSQEAQEETDIQLLIAQQQQKQRELKYNLLKLISYMKEQQNMLKL
HTGMLN
>MN777719.1|Gamma|319_2301_1_fow
MPFWWGRRNRFWYKNNYRRRAYRRKRKRWPKRRRRNFRRPTRRRRRRRRKQKVRKKKQTLTLKVWQPDTIRKCK
IKGTGIHIVGGNGTQYRCFTANRFDWTPPLMPGGGGFGVEKFTLQHLYNEHLLSNNIWTQSNKMLDLVRYTGCR
IKFYRHETIDFIASYNRNLPFNLEKYTYADSHPNSLLHSRHKKLIPSLKTRRSGKRYITMKIKPPKPVSNKWFF
QETFSSTPLFKLITSIIDLRYPHISQIGSNELVSLFCLNTDFYANAGWGNVHPPGTHSKWYVPYHNIAAITTVV
IKGRDTQITVDGSDYKKSVSYETGYFQPKLLQAEKIKNTAMENIPIQASRYNPTKDDGVGNKLWLVNVLNYSFA
PPKIDADLYIENLPLWQLVWGFTDFVKQTKKDSTFLNTYYAAFESKYLEPAVSTSKIYIPIDMSFIQGKGPYDS
YVSNYRKQNWYPILNAQLKILNAIAQAGPFVPKLDNIFLSSWELYSNYTFYFKFGGSELPEPDTVNPQDQGTYV
IPTNIQKAIQVSNPQKQSPYTTLHSWDYRRGLITSSAFKRMCENQTTDTEFEPDTETIPKKKKKIQHGNQLQRA
EEETQEIQNCLLSLCEENTFQETPQTSLLELIQQQQQQQQQLKHNLLKLLSDLKLKQRSLQLQTGLIH

### Example 41: Evasion of Immune System

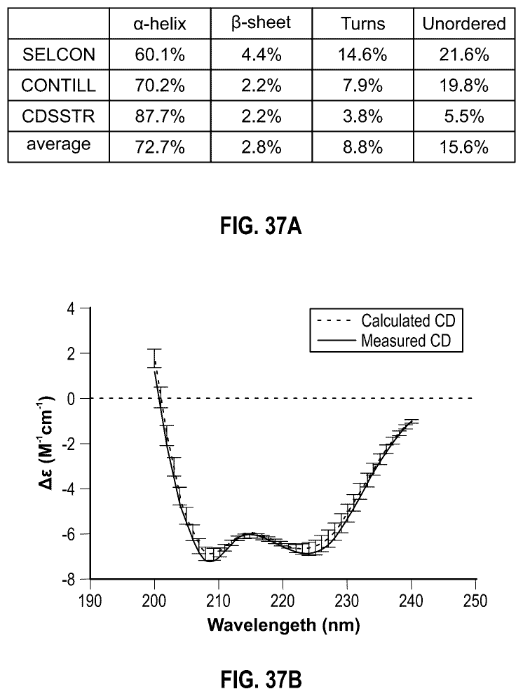

This Example describes the anellovirus C-terminal region and its relation to immune evasion. Absent from the Ring 10 structure is the majority of the C-terminal region (residues 545-672). Observed density for residues 545-562 showed that, were the C-terminal region present on the wild-type virus, the C-terminus would extend from the JR core toward the surface near the 3-fold axis between the spike domain crowns. The C-terminal region is semi-conserved between all anelloviruses, is predicted to be helical in nature, and has a glutamate-rich region (residues 636-640 on Ring 10) which could N-terminally cap a conserved helical motif. To determine if the C-terminal residues of Ring 10 would form a helical structure, circular dichroism experiments were performed, as described in Example 37, on the C-terminal peptides used to generate the aforementioned antibodies. Indeed, analysis with circular dichroism suggested that the C-terminal region (residues 635-672) is helical in solution, suggesting the C-terminal region would form a coiled-coil domain (FIGS. 37A and 37B), strongly suggesting a leucine-zipper like association of the helices perhaps due to the periodicity of hydrophobic residues. Electron microscopy of full-length Ring 10 ORF1 vs ORF1 which either had the C-terminus proteolytically removed or genetically removed suggested that particle formation is improved with the absence of the semi-conserved C-terminus (FIG. 34A-34C). Given that anelloviruses have evolved to evade the immune system, and that the C-terminal region is the immunodominant region of ORF1 and may be antagonistic to particle formation, the semi-conserved C-terminal region may be required for wild-type particle assembly but processed during viral particle maturation.

Despite anelloviruses constituting the majority of the human virome, their capsid structure was unknown. Determination of the Ring 10 structure as described by the examples included herein demonstrates that ORF1 encodes the capsid protein and that anelloviruses evolved a novel spike domain that extends around the 5-fold axis to form a crown structure. These crowns are capped with hypervariable P2 regions, which likely inhibits the development of antibodies against the better conserved spike P1 domain via steric hindrance. Analysis of the P1 surface revealed conserved residue patches that may have a receptor binding function.

The structure of the Ring 10 Betatorquevirus can be used to guide future anellovirus research. In particular, the diversity and immunological stealth of anelloviruses indicates that they could be used to deliver therapeutic genes to cell types not currently addressed by existing vectors, and that they may be less susceptible to pre-existing immunity or to the development of neutralizing antibodies following initial treatment. The availability of a capsid structure will help guide the design of anellovirus-based gene therapy vectors.

### Example 42: Production and Purification of AnelloVLPs

### Ring2 AnelloVLP:

A plasmid that encodes Ring2 ORF1 with a deletion of the C-terminus amino acids 611-666 (pRTx-2652; see also Table X1 construct listed as Ring2delCterm (A611-666) (7047)) was transfected into Expi293 cells. The cells were collected and resuspended in lysis buffer (50 mM Tris HCl, pH 8, 250 mM NaCl, 2 mM Magnesium Chloride). EDTA (0.5M) and protease inhibitor were added, and the cells were lysed using a microfluidizer. After lysing, protein inhibitor and triton X were added to the lysate and incubated for 30 minutes. The lysate was centrifuged at 12100 rpm and the supernatant was collected.

The lysate supernatant in 50 mM Tris HCl pH8, 250 mM NaCl was passed through a HiTrap heparin HP column (Cytiva) to separate 60mer VLPs from other smaller proteins from the mixture. The VLP fractions were dialyzed into Capto buffer (50 mM Tris HCl pH 8, 100 mM NaCl) overnight and incubated for 1.5 hrs with Capto400 resin at a 1:10 ratio of resin to protein. The preparation was spun down and supernatant collected.

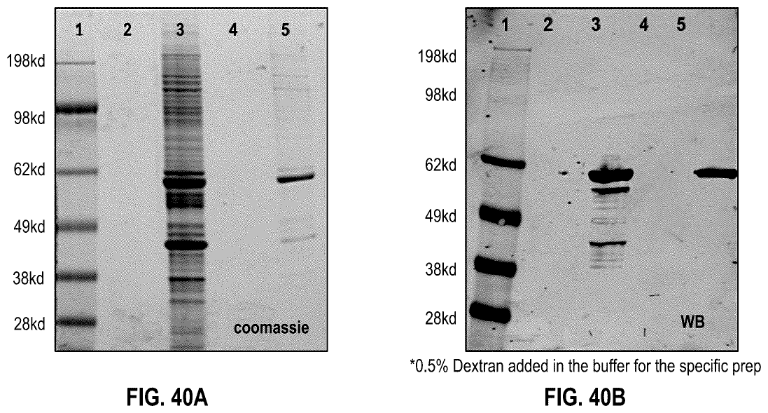

Samples were loaded onto a gel and Coomassie stained (FIG. 40A) or a Western blot was performed using a Ring2 HVR Linker primary antibody raised against amino acids 284-319 of the HVR region of Ring2 ORF1 and a goat-anti-rabbit secondary antibody (FIG. 40B). The lanes were as follows:
Lane 1: Ladder
Lane 2: Blank
Land 3: 2652 unpurified material
Lane 4: Blank
Lane 5: Ring2 AnelloVLP 2652 after Capto400 purification

Both gels show a relatively pure preparation of Ring2 VLPs after Capto400 purification (see lane 5).

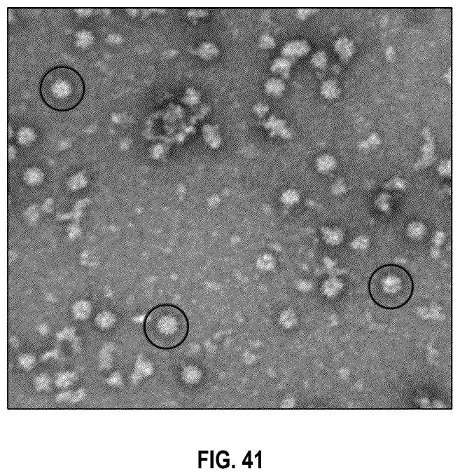

Electron microscopy of the preparation obtained after Capto400 purification confirmed Ring2 VLP formation at 65000× magnification (FIG. 41).

### Ring]9 AnelloVLP:

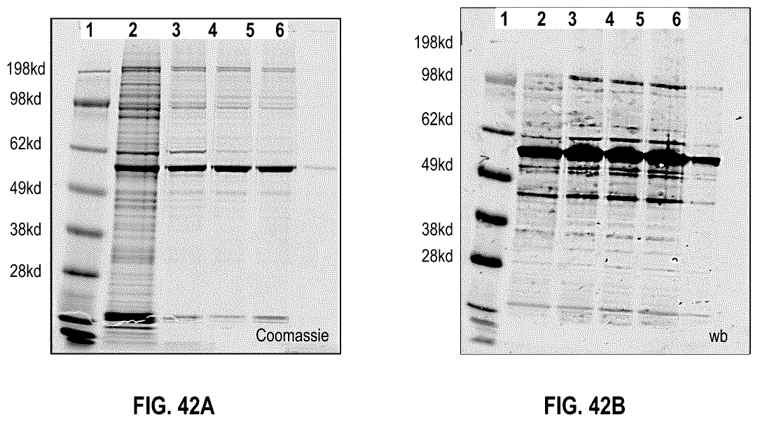

A plasmid that encodes Ring19 ORF1 with a deletion of the C-terminus amino acids 600-655 (pRTx-2814; see Table X1, construct Ring19delCterm (A600-655) (7120)) was similarly transfected as above into expi293 cells. The cells were collected and resuspended in lysis buffer (50 mM Tris HCl, pH 8, 250 mM NaCl, 2 mM Magnesium Chloride). EDTA (0.5M) and protease inhibitor were added, and the cells were lysed using a microfluidizer. After lysing, protein inhibitor and triton X were added to the lysate and incubated for 30 minutes. The lysate was centrifuged at 12100 rpm and the supernatant was collected. The supernatant was then purified through the HiTrap Heparin HP column and dialyzed into the Capto buffer as described above. The VLP preparation was then purified through a Capto400 column to further separate the VLPs from smaller proteins. Fractions “C12”, “D1”, and “D2” are different fractions collected during purification that showed peaks on the chromatogram. Samples from fractions C12, D1, and D2 were loaded onto a gel and Coomassie stained (FIG. 42A) or a Western blot was performed using a Ring19 HVR3 primary antibody that was raised against amino acids 342-352 of the Ring19 ORF1 HVR region (FIG. 42B). The lanes were as follows:
Lane 1: Ladder
Lane 2: Ring19 AnelloVLPs post heparin purification
Lane 3: Ring19 AnelloVLPs post dialysis
Lane 4: Ring19 AnelloVLPs capto400 C12 fraction
Lane 5: Ring19 AnelloVLPs capto400 D1 fraction
Lane 6: Ring19 AnelloVLPs capto400 D2 fraction

Both gels show Ring19 VLPs after capto400 purification.

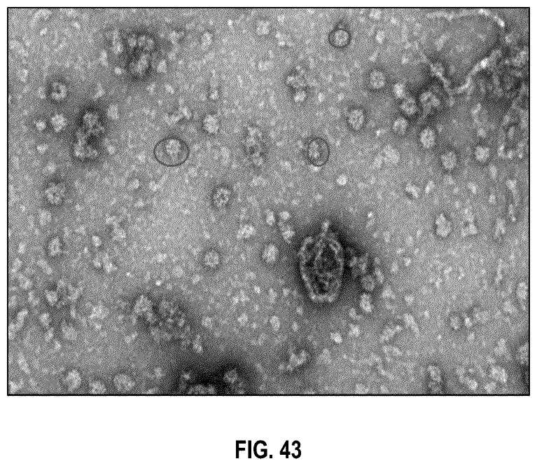

Electron microscopy of the preparation obtained after Capto400 purification confirmed Ring19 VLP formation at 65000× magnification (FIG. 43).

### Example 43: Conjugation of AnelloVLPs

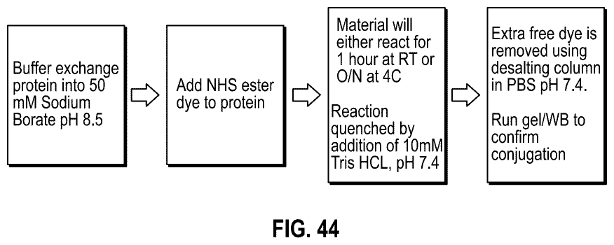

In this example, NHS ester moieties were conjugated via click chemistry to the surface lysines of the AnelloVLPs prepared according to the example above. The conjugation workflow is shown in FIG. 44.

The preparations of the AnelloVLPs prepared above were buffer exchanged into 50 mM sodium borate pH8.5 overnight and NHS ester 647-Alexa FluorT^{M} (“NHS Ester 647”) (Succinimidyl Ester), (ThermoFisher Catalog number: A20006) (for Ring2 AnelloVLPs) or EZ-Link™ NHS-Biotin (“NHS-Biotin”) (ThermoFisher Catalog number: 20217) (for Ring19 AnelloVLPs) were added in varying VLP: NHS Ester molar ratios and reacted for 1 hr at room temperature or overnight at 4° C. The reactions were stopped by addition of 10 mM Tris HCl, pH7.4. The preparations were further desalted in PBS pH7.4 to remove extra free NHS ester moieties. Gels or Western blots were run to confirm conjugation.

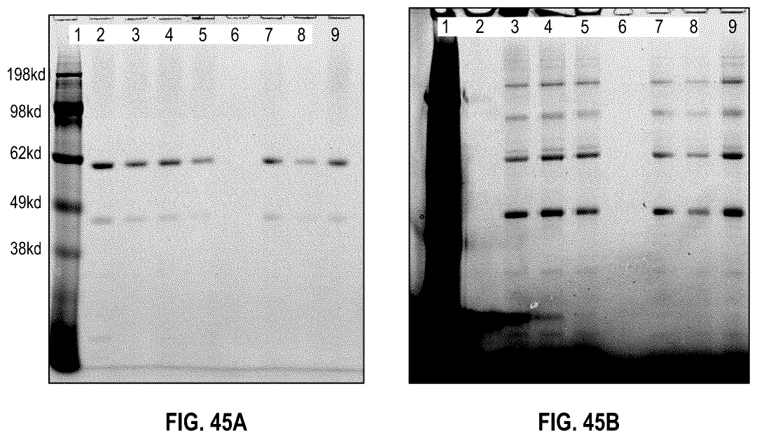

FIG. 45A shows a Coomassie stained gel and FIG. 45B shows a gel scanned under 520 nm of Ring2 AnelloVLP conjugated with NHS Ester 647. The lanes shown are as follows:
Lane 1: ladder
Lane 2: Ring2 AnelloVLP 2652 unlabeled
Lane 3: Ring2 AnelloVLP 2652 labeled at 1:5 VLP:NHS Ester 647 molar ratio, no desalt
Lane 4: Ring2 AnelloVLP 2652 labeled at 1:10 VLP:NHS Ester 647 molar ratio, no desalt
Lane 5: Ring2 AnelloVLP 2652 labeled at 1:20 VLP:NHS Ester 647 molar ratio, no desalt
Lane 6: Blank
Lane 7: Ring2 AnelloVLP 2652 labeled at 1:5 VLP:NHS Ester 647 molar ratio, desalted
Lane 8: Ring2 AnelloVLP 2652 labeled at 1:10 VLP:NHS Ester 647 molar ratio, desalted
Lane 9: Ring2 AnelloVLP 2652 labeled at 1:20 VLP:NHS Ester 647 molar ratio, desalted

The Coomassie-stained gel shows Ring2 AnelloVLPs at around 62kD as expected in all protein-containing lanes. The gel scanned under 520 nm shows no visible band in lane 2 (unlabeled Ring2 AnelloVLP) while the other lanes containing labeled AnelloVPs show visible bands at about 62kD, confirming the conjugation of Ring2 AnelloVLPS with NHS Ester 647.

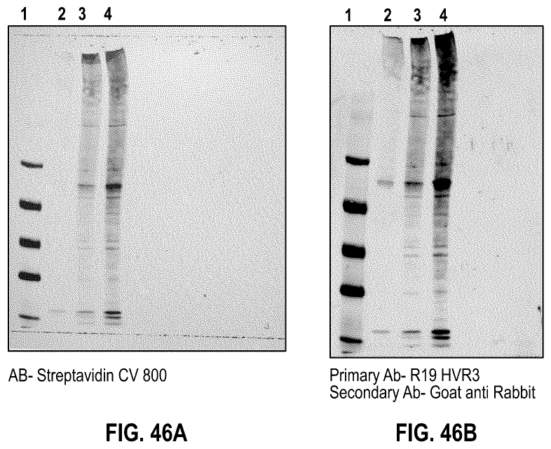

FIG. 46A shows a Western blot with a streptavidin CV 800 antibody [Licor Bioscience—Catalog number P/N: 926-32211]and FIG. 46B shows a Western blot with the R19 HVR3 primary antibody described above and a goat-anti rabbit secondary antibody. The lanes shown are as follows:
Lane 1: Ladder
Lane 2: Ring19 AnelloVLP unlabeled
Lane 3: Ring19 AnellovLP labeled at 1:5 VLP:NHS Ester biotin molar ratio [desalted]
Lane 4: Ring19 AnellovLP labeled at 1:10 VLP:NHS Ester biotin molar ratio [desalted]

The Western blot using the Ring19 specific antibody shows visible bands at around 62kD in both the unlabeled and labeled Ring19 AnelloVLP samples. The Western blot with streptavidin shows a band at around 62kD with Ring19 AnelloVLPs labeled with NHS Ester biotin while the unlabeled Ring19 AnelloVLP shows no visible band, confirming the conjugation of Ring19 AnelloVLPs with NHS Ester biotin.

### Example 44: VLP Conjugation to SARS-CoV-2 RBD Peptide

In this Example, a His-Tag recombinant receptor binding domain (RBD) comprising amino acids 319-541 from SARS-CoV-2 coronavirus spike protein (ThermoFisher Catalog #RP-87704) (referred to in this example as RBD) was conjugated to the surface of AnelloVLPs. As shown in FIG. 47, the conjugation involved a first step of conjugating NHS-DBCO to the surface ORF1 of anelloVLPs. Azide-labeled RBD effectors were then conjugated to the anelloVLPs via the DBCO linker. This approach can be generalized to attach azide-labeled effectors (e.g., antigenic peptides and proteins) to anelloVLPs with DBCO linkers on their surfaces.

First, RBD was labeled with either DBCO or Azide to confirm that RBD could be labeled for future conjugation to AnelloVLPs. In brief, 100 μg of the His-tagged recombinant RBD (ThermoFisher Catalog #RP-87704) was resuspended in 1 ml of sodium borate buffer (pH=8.5) to final concentration of 0.1 mg/ml. The RBD amino acids from 319-541 contains 11 lysines that could potentially be labeled by NHS. Thus, in one condition, 2 μl of DBCO-PEG4-NHS 1 mg/ml was added to 200 μl of 0.1 mg/ml RBD and incubated overnight at 4° C. (2 pg DBCO-PEG4-NHS: 20 μg RBD). In a second condition, 2 μl of Azide-PEG4-NHS at 1 mg/ml was added to 200 μl of 0.1 mg/ml RBD and then incubated overnight at 4° C. (2 μg Azide-PEG4-NHS: 20 μg RBD). The next day, a zeba desalting column was prepared by opening the bottom of a column and placing it into a microfuge tube, spinning at 1500g for 1 min at 4° C., adding 400 μl of Borate buffer (50 mM sodium borate) and spinning at 1500g for one minute and then discarding the flow-through. This process is then repeated 3˜4 times and the column is then placed into a fresh microfuge tube.

Unreacted DBCO/Azide was desalted out of the samples by running through a desalting column for two minutes at 1500g and at 4° C. Samples were then split as follows:
a. 100 μl RBD-PEG4-DBCO
b. 100 μl RBD-PEG4-DBCO+CaIFlour488
c. 100 μl RBD-PEG4-Azide
d. 100 μl RBD-PEG4-Azide+Alexa488 sDIBO

RBD-PEG4-DBCO+CaIFlour488 and RBD-PEG4-Azide+Alexa488 sDIBO were each made by adding 1 μl of CaIFluor488 Azide/Clicklt Alexa488 sDIBO to the RBDs. The mixture was incubated for 30 minutes, and this process was then repeated three times. RBD-PEG4-DBCO+CaIFlour488 and RBD-PEG4-Azide+Alexa488 sDIBO samples were then desalted as described. A western blot was then run to determine whether RBD labeling occurred. As shown in FIG. 48, labeling of RBD was confirmed with both DBCO and azide for future VLP conjugation. This demonstrated that a linker could be conjugated to the RBD.

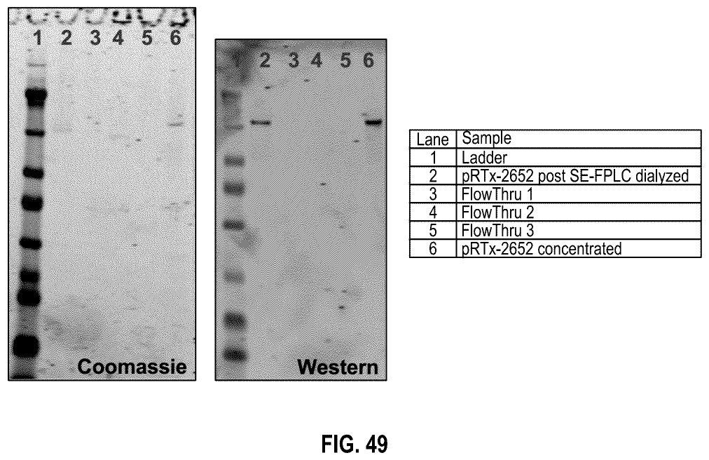

To conjugate RBD to a Ring2 VLP, a fraction block of Ring2 ORF1 with a C-terminal deletion of amino acids 611-666 (pRTx-2652; corresponding to Ring2delCterm (A611-666) (7047) as listed in Table X1) in TBS was produced using SE-FPLC. Fractions were pooled and dialyzed into 2L of PBS+0.01% poloxamer at 4° C. After 3 hours, buffer was refreshed. This was then repeated twice more and left overnight at 4° C. Approximately 30 ml of the post-SE-FPLC solution was concentrated to approximately 1.5 ml final volume. A Western blot was run to confirm that the sample was retained (FIG. 49). The next day, approximately 1.5 ml of SE-FPLC pRTx-2652 was concentrated down to 220 μl.

1 μl NHS-PEG4-DBCO was added to 100 μl SE-FLPC pRTx-2652 samples generated as described above. The samples were then incubated for 1 hour at room temperature. 3 μl 1M Tris pH 8 was added, and the samples were desalted. RBD was labeled with NHS-PEG4-Azide by resuspending RBD in 200 μl borate buffer (0.5 mg/mL final) and 1 μl NHS-PEG4-Azide for 1 hour at room temperature. 3 μl 1M Tris pH 8 was then added and the RBD sample was desalted.

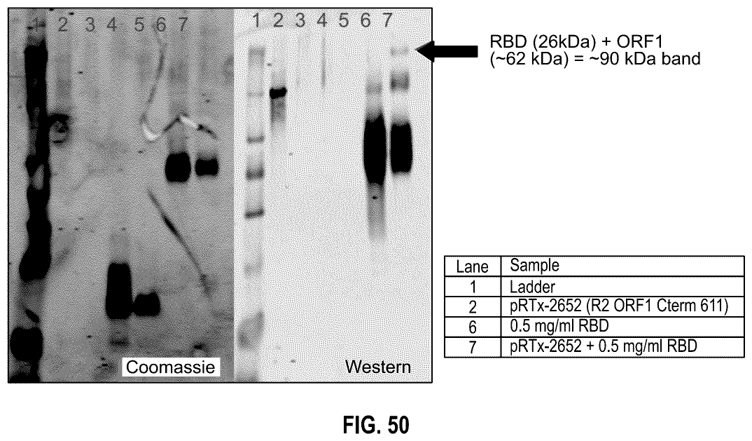

7.5 μl of the RBD peptide was then added to 30 μl of DBCO-2652 to produce a final peptide concentration 0.1 mg/ml. The sample was then incubated at 37° C. for 2.5 hours and conjugation was assessed by Coomassie staining and Western blot with an anti-Ring2 ORF1 antibody. As shown in FIG. 50, a 90 kDa band corresponding to the conjugation of RBD (26 kDa) to pRTx-2652 anelloVLPs (˜62 kDa) was observed.

What is claimed is:

1. A mixture comprising:
a plurality of Anellovirus ORF1 molecules, and
a chaotropic agent;
wherein at least 75% of the plurality of Anellovirus ORF1 molecules in the mixture are comprised in capsomers; and
wherein host cell DNA in the mixture is substantially dissociated from the plurality of Anellovirus ORF1 molecules.

2. The mixture of claim 1, wherein the mixture comprises a plurality of pentamers comprising the Anellovirus ORF1 molecules.

3. The mixture of claim 1, wherein the mixture comprises a plurality of decamers comprising the Anellovirus ORF1 molecules.

4. The mixture of claim 1, which does not comprise a proteinaceous exterior comprising about 40-80 copies of an Anellovirus ORF1 molecule.

5. The mixture of claim 1, wherein the Anellovirus ORF1 molecules comprise an arginine-rich region.

6. The mixture of claim 1, wherein the Anellovirus ORF1 molecules do not comprise a C-terminal domain.

7. The mixture of claim 1, which is a cell-free system.

8. The mixture of claim 1, wherein the Anellovirus ORF1 molecules each comprise a tagging moiety.

9. The mixture of claim 1, wherein the tagging moiety comprises chitin binding protein (CBP), maltose binding protein (MBP), glutathione-S-transferase (GST), and poly(His) tag.

10. The mixture of claim 9, wherein the mixture comprises a plurality of pentamers comprising the Anellovirus ORF1 molecules.

11. The mixture of claim 9, wherein the mixture comprises a plurality of decamers comprising the Anellovirus ORF1 molecules.
